(19) United States
(12) Patent Application Publication
(10) Pub. No.: US 2024/0408096 A1
(43) Pub. Date: Dec. 12, 2024

(54) DOSING REGIMENS FOR ORAL ALK2 KINASE INHIBITORS

(71) Applicant: BioCryst Pharmaceuticals, Inc., Durham, NC (US)

(72) Inventors: Yarlagadda S. Babu, Birmingham, AL (US); William P. Sheridan, Montecito, CA (US)

(21) Appl. No.: 18/268,688

(22) PCT Filed: Dec. 20, 2021
(86) PCT No.: PCT/US21/64333

§ 371 Date: Jun. 21, 2023

Related U.S. Application Data
(60) Provisional application No. 63/128,635, filed on Dec. 21, 2020.

(51) Int. Cl. A61K 31/519; A61K 9/48

(52) U.S. Cl. CPC A61K 31/519; A61K 9/4825

(57) ABSTRACT

Disclosed are dosage forms of compounds and pharmaceutically acceptable salts thereof, which are inhibitors of ALK2 kinase, including mutant forms of ALK2. Also disclosed are methods of using the oral dosage forms in the treatment or prevention of a disease or condition whose treatment would benefit from ALK2 kinase inhibition (e.g., Fibrodysplasia Ossificans Progressiva and cancer).

### RELATED APPLICATIONS

This application claims the benefit of priority to U.S. Provisional Patent Application No. 63/128,635, filed Dec. 21, 2020.

### BACKGROUND

A single mutation (R206H) within the kinase domain of one (ACVR1/ALK2) of the four human bone morphogenetic protein (BMP) receptors has been linked to a catastrophic disorder of secondary (heterotopic) bone formation. As a result of the mutation, all children presenting with features of classic Fibrodysplasia Ossificans Progressiva (FOP) eventually become encased in, and their movement blocked by, a second heterotopic skeleton. This process is referred to as heterotopic (extra-skeletal) ossification (HO). The disorder has long been associated with dysregulation of BMP signaling in soft tissues (skeletal muscle, tendon, ligament, fascia) that were transformed into ribbons, sheets and plates of heterotopic bone via an endochondral process. In addition to the common R206H mutation linked to the classic form of FOP, other dysregulating mutations have been identified in ACVR1/ALK2 that lead to atypical and variant forms of FOP. Further, compounds effective in regulating BMP signaling based on their ability to inhibit ALK2 have been shown also to inhibit kinases from multiple signaling pathways.

Other disease and conditions involve hyperactivation of ALK2 kinase include, but are not limited to, cancer.

Thus there remains a need for effective methods of treatment and effective dosing regimens for the treatment of diseases and conditions whose treatment would benefit from ALK2 kinase inhibition (meaning that in such disease or condition it would be desirable to reduce ALK2 kinase activity).

### SUMMARY OF THE DISCLOSURE

The present disclosure provides dosage forms, particularly oral dosage forms, of a compound of the disclosure and methods or treatment utilizing such dosage forms.

In one aspect, the disclosure provides an oral dosage form comprising a compound of the disclosure (wherein a compound of the disclosure is a compound from Table 1 as described herein, or a pharmaceutically acceptable salt thereof) and a pharmaceutically acceptable carrier. In certain embodiments, the oral dosage form is a capsule, such as a hard or soft gelatin capsule.

In certain embodiments, a compound of the disclosure inhibits ALK2(R206H) activity with a potency (IC₅₀) of less than 100 nM (as described in PCT Application No. PCT/US2018/037503, which is incorporated by reference for such teaching).

In another aspect, the disclosure provides methods of treating or preventing a disease or condition whose treatment would benefit from ALK2 kinase inhibition (meaning that in such disease or condition it would be desirable to reduce ALK2 kinase activity), the method comprising orally administering to a subject in need thereof a therapeutically effective amount of a compound of the disclosure, or a pharmaceutically acceptable salt thereof. In certain embodiments, the disease or condition is FOP. In certain embodiments, the disease or condition is cancer.

Exemplary cancers that may be treated with a compound of the disclosure include, but are not limited to, tumors of the central nervous system, breast cancer, prostate cancer, skin cancer (including, but not limited to, basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcoma, soft tissue sarcomas, osteosarcomas, peripheral primitive neuroectodermal tumors, retinoblastomas, rhabdomyosarcomas, and Wilms tumors.

### BRIEF DESCRIPTION OF THE DRAWINGS

FIG. 1 shows that Compound A exhibits dose proportionality in plasma concentration-time curves following oral administration of single ascending doses from 5 mg to 25 mg in Part 1 of the clinical study described herein in healthy subjects (arithmetic mean±SD).

FIG. 2 shows that exposure (single dose Cₘₐₓ) to Compound A across the single ascending oral dose cohorts of Part 1 of the clinical study described herein in healthy subjects was approximately linear and dose-proportional.

FIG. 3 shows that exposure (single dose AUC₀₋₂₄) to Compound A across the single ascending oral dose cohorts of Part 1 of the clinical study described herein in healthy subjects was approximately linear and dose-proportional.

FIG. 4 shows mean plasma Compound A concentration-time profile on a linear scale following oral administration single ascending doses of Compound A from 5 mg to 25 mg in Part 1 of the clinical study described herein in healthy subjects (arithmetic mean±SD).

FIG. 5 shows mean (±SD) plasma Compound A concentration-time profile on a logarithmic scale following oral administration single ascending doses of Compound A from 5 mg to 25 mg in Part 1 of the clinical study described herein in healthy subjects (arithmetic mean±SD).

FIG. 6 shows that a Compound A exhibits dose proportionality in plasma concentration-time curves following oral administration of multiple ascending doses from 5 mg to 20 mg in Part 2 of the clinical study described herein in healthy subjects (arithmetic mean±SD).

FIG. 7 shows that exposure (steady state Cₘₐₓ) to Compound A across the multiple ascending oral dose cohorts of Part 2 of the clinical study described herein in healthy subjects was approximately linear and dose-proportional.

FIG. 8 shows that exposure (steady state AUCₜₐᵤ) to Compound A across the multiple ascending oral dose cohorts of Part 2 of the clinical study described herein in healthy subjects was approximately linear and dose-proportional.

FIG. 9 shows mean plasma Compound A concentration-time profile on a linear scale on days 1 and 7 following oral administration of the first dose of Compound A on day 1 and after the last dose of Compound A on day 7 (arithmetic mean±SD).

FIG. 10 shows mean plasma Compound A concentration-time profile on a logarithmic scale on days 1 and 7 following oral administration of the first dose of Compound A on day 1 and after the last dose of Compound A on day 7 (arithmetic mean±SD).

FIG. 11 shows a plot of mean trough plasma levels of Compound A over time.

### DETAILED DESCRIPTION

The results of nonclinical pharmacology, pharmacokinetics (PK), and toxicology studies of show the compounds of the disclosure are potent, small molecule inhibitor of ALK2 and may be used to treat diseases and conditions whose treatment would benefit from ALK2 kinase inhibition (meaning that in such disease or condition it would be desirable to reduce ALK2 kinase activity). Such diseases and conditions include, but are not limited to, fibrodysplasia ossificans progressive (FOP) and cancer.

The compounds of the disclosure are potent inhibitors of the native ALK2 enzyme and the mutant ALK2(R206H) enzyme, the latter of which underlies the pathology of FOP. A preferred compound of the disclosure is “Compound A” (also known as BCX9250). In several functional assays, Compound A inhibits ALK2(R206H) activity and wtALK2 activity with a potency (IC₅₀) of less than 100 nM. In a mouse model of HO bone formation, a significant reduction in new HO bone formation was observed following Compound A dosing for 21 days.

Compound A exhibited minimal off-target effects in a screening study of G-protein coupled receptor (GPCRs) related kinases. In addition, no adverse central nervous system, respiratory, or cardiovascular issues were revealed for Compound A in safety pharmacology studies at human equivalent doses (HEDs) that exceeded likely human daily clinical dose ranges (such as, but not limited to, 5 to 200 mg). Oral administration of Compound A to dogs did not induce prolongation of corrected QT interval (QTc) or other changes in quantitative electrocardiogram (ECG) parameters at HEDs up to 667 mg based on a 60 kg subject (3.3-times higher than a clinical dose of 200 mg).

The nonclinical PK studies demonstrate that compounds of the disclosure, including Compound A, are orally bioavailable and do not accumulate with repeated dosing for up to 28 days in toxicokinetic assessments. In vitro studies show that Compound A is highly protein-bound in rat, dog, monkey, and human plasma (≥99.9% in all species evaluated) and is metabolized by animal and human liver S9 fraction with no apparent human-specific metabolites. In vitro studies demonstrate that Compound A is not an inhibitor of cytochrome P450 (CYP)1A2, CYP2A6, CYP2B6, CYP2E1 and CYP3A, and is an inhibitor of CYP2C8, CYP2C9, CYP2C19 and CYP3A4.

The toxicological profile of Compound A has been well characterized in oral studies in rats and dogs, and in vitro and in vivo genotoxicity studies. Compound A was negative for the induction of numerical chromosomal aberrations and negative in the bacterial reverse mutation (AMES) assay, and negative in vivo in the rat micronucleus-comet genotoxicity assays.

### Definitions

The articles “a” and “an” are used herein to refer to one or to more than one (i.e., to at least one) of the grammatical object of the article. By way of example, “an element” means one element or more than one element.

Unless otherwise defined, all technical and scientific terms used herein have the same meaning as commonly understood by one of ordinary skill in the art to which this invention belongs. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000). Although methods and materials similar or equivalent to those described herein can be used in the practice or testing of the present invention, suitable methods and materials are described below.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of a compound of the disclosure. As another example, the salt may comprise less than one inorganic or organic acid molecule per molecule of base, such as two molecules of a compound of the disclosure per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, tale, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” or “treating” as used herein means prevent, halt or slow the progression of, or eliminate a disease or condition in a subject. In one embodiment “treat” or “treating” means halt or slow the progression of, or eliminate a disease or condition in a subject. In one embodiment, “treat” or “treating” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In one embodiment “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

“Administering” or “administration of” a compound of the disclosure to a subject can be carried out using one of a variety of methods known to those skilled in the art. For example, a compound of the disclosure can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound of the disclosure can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a compound of the disclosure to a subject will also depend, for example, on the age and/or the physical condition of the subject and the chemical and biological properties of the compound or agent (e.g., solubility, digestibility, bioavailability, stability and toxicity). In some embodiments, a compound of the disclosure is administered orally, e.g., to a subject by ingestion. In some embodiments, the orally administered compound of the disclosure is in an extended release or slow release formulation, or administered using a device for such slow or extended release.

### Dosage Forms of the Disclosure

One aspect of the disclosure provides dosage forms of small molecules that inhibit human ALK2 kinase, including an ALK2 kinase comprising a mutation, particular an activating mutation, such as R206H.

In a particular aspect, the disclosure provides oral dosage forms of small molecules that inhibit human ALK2 kinase, including an ALK2 kinase comprising a mutation, particular an activating mutation, such as R206H.

The dosage forms described herein, particularly oral dosage forms, comprise a compound of the disclosure as set forth in table 1 below:

TABLE 1

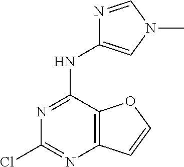 [SMILES: Cn1cnc(Nc2nc(Cl)nc3ccoc23)c1]

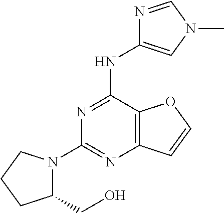 [SMILES: Cn1cnc(Nc2nc(N3CCC[C@H]3CO)nc3ccoc23)c1]

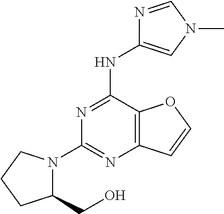 [SMILES: Cn1cnc(Nc2nc(N3CCC[C@@H]3CO)nc3ccoc23)c1]

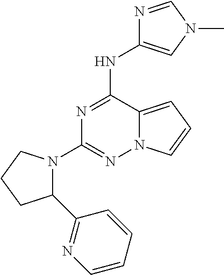 [SMILES: Cn1cnc(Nc2nc(N3CCCC3c3ccccn3)nn3cccc23)c1]

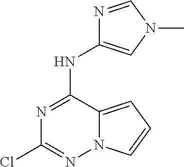 [SMILES: Cn1cnc(Nc2nc(Cl)nn3cccc23)c1]

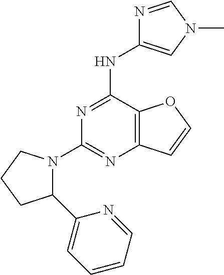 [SMILES: Cn1cnc(Nc2nc(N3CCCC3c3ccccn3)nc3ccoc23)c1]

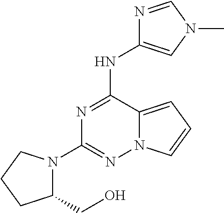 [SMILES: Cn1cnc(Nc2nc(N3CCC[C@H]3CO)nn3cccc23)c1]

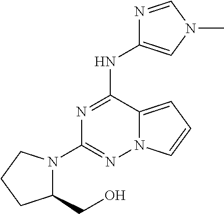 [SMILES: Cn1cnc(Nc2nc(N3CCC[C@@H]3CO)nn3cccc23)c1]

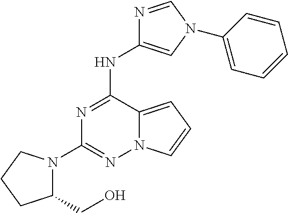 [SMILES: OC[C@@H]1CCCN1c1nc(Nc2cn(-c3ccccc3)cn2)c2cccn2n1]

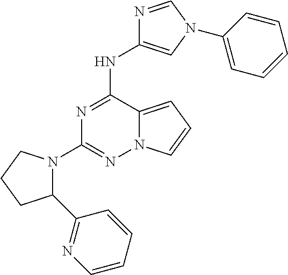 [SMILES: c1ccc(-n2cnc(Nc3nc(N4CCCC4c4ccccn4)nn4cccc34)c2)cc1]

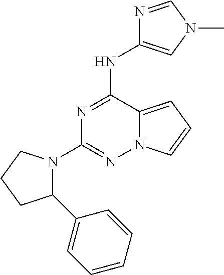 [SMILES: Cn1cnc(Nc2nc(N3CCCC3c3ccccc3)nn3cccc23)c1]

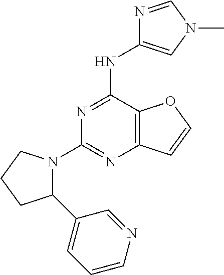 [SMILES: Cn1cnc(Nc2nc(N3CCCC3c3cccnc3)nc3ccoc23)c1]

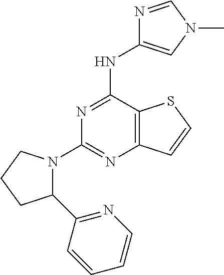 [SMILES: Cn1cnc(Nc2nc(N3CCCC3c3ccccn3)nc3ccsc23)c1]

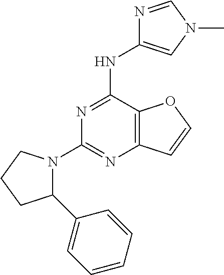 [SMILES: Cn1cnc(Nc2nc(N3CCCC3c3ccccc3)nc3ccoc23)c1]

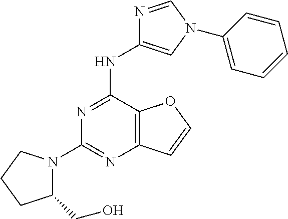 [SMILES: OC[C@@H]1CCCN1c1nc(Nc2cn(-c3ccccc3)cn2)c2occc2n1]

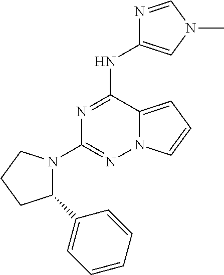 [SMILES: Cn1cnc(Nc2nc(N3CCC[C@H]3c3ccccc3)nn3cccc23)c1]

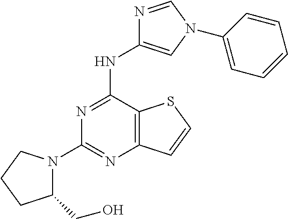 [SMILES: OC[C@@H]1CCCN1c1nc(Nc2cn(-c3ccccc3)cn2)c2sccc2n1]

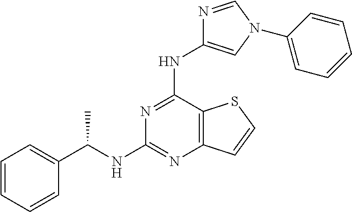 [SMILES: C[C@H](Nc1nc(Nc2cn(-c3ccccc3)cn2)c2sccc2n1)c1ccccc1]

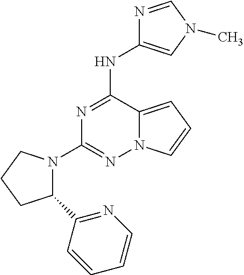 [SMILES: Cn1cnc(Nc2nc(N3CCC[C@H]3c3ccccn3)nn3cccc23)c1]

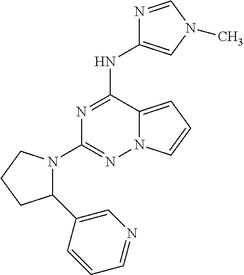 [SMILES: Cn1cnc(Nc2nc(N3CCCC3c3cccnc3)nn3cccc23)c1]

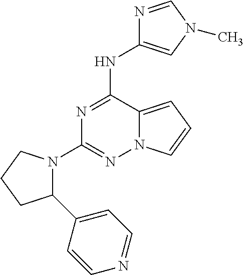 [SMILES: Cn1cnc(Nc2nc(N3CCCC3c3ccncc3)nn3cccc23)c1]

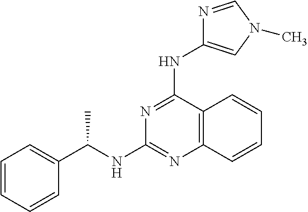 [SMILES: C[C@H](Nc1nc(Nc2cn(C)cn2)c2ccccc2n1)c1ccccc1]

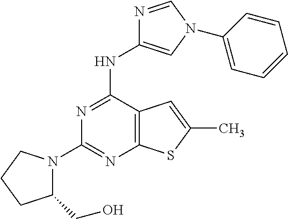 [SMILES: Cc1cc2c(Nc3cn(-c4ccccc4)cn3)nc(N3CCC[C@H]3CO)nc2s1]

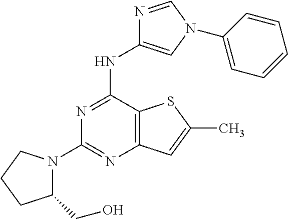 [SMILES: Cc1cc2nc(N3CCC[C@H]3CO)nc(Nc3cn(-c4ccccc4)cn3)c2s1]

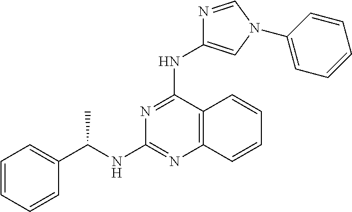 [SMILES: C[C@H](Nc1nc(Nc2cn(-c3ccccc3)cn2)c2ccccc2n1)c1ccccc1]

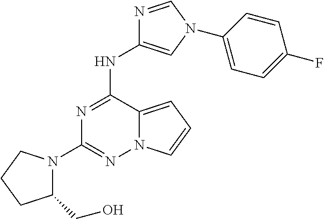 [SMILES: OC[C@@H]1CCCN1c1nc(Nc2cn(-c3ccc(F)cc3)cn2)c2cccn2n1]

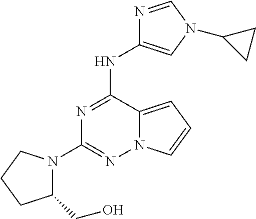 [SMILES: OC[C@@H]1CCCN1c1nc(Nc2cn(C3CC3)cn2)c2cccn2n1]

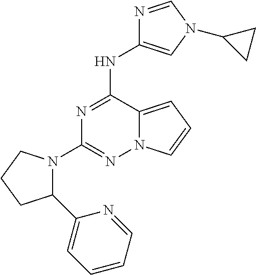 [SMILES: c1ccc(C2CCCN2c2nc(Nc3cn(C4CC4)cn3)c3cccn3n2)nc1]

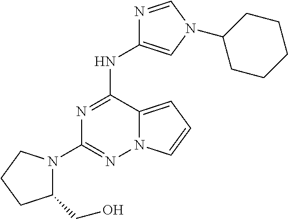 [SMILES: OC[C@@H]1CCCN1c1nc(Nc2cn(C3CCCCC3)cn2)c2cccn2n1]

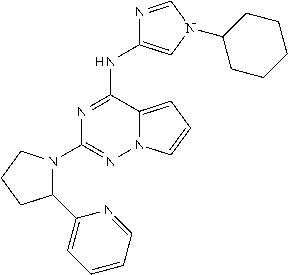 [SMILES: c1ccc(C2CCCN2c2nc(Nc3cn(C4CCCCC4)cn3)c3cccn3n2)nc1]

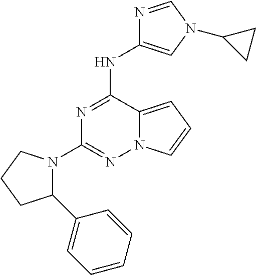 [SMILES: c1ccc(C2CCCN2c2nc(Nc3cn(C4CC4)cn3)c3cccn3n2)cc1]

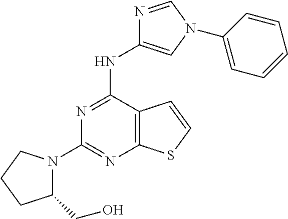 [SMILES: OC[C@@H]1CCCN1c1nc(Nc2cn(-c3ccccc3)cn2)c2ccsc2n1]

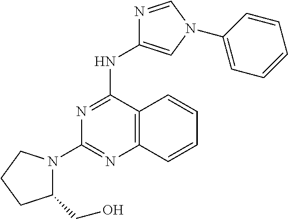 [SMILES: OC[C@@H]1CCCN1c1nc(Nc2cn(-c3ccccc3)cn2)c2ccccc2n1]

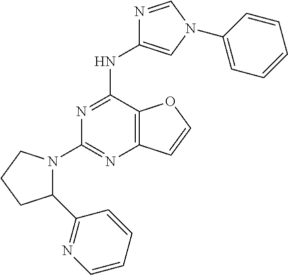 [SMILES: c1ccc(-n2cnc(Nc3nc(N4CCCC4c4ccccn4)nc4ccoc34)c2)cc1]

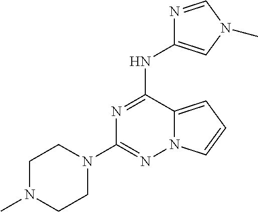

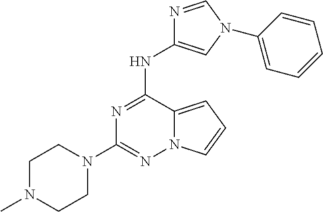 [SMILES: CN1CCN(c2nc(Nc3cn(-c4ccccc4)cn3)c3cccn3n2)CC1]

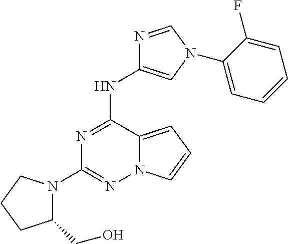 [SMILES: OC[C@@H]1CCCN1c1nc(Nc2cn(-c3ccccc3F)cn2)c2cccn2n1]

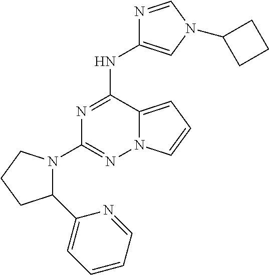 [SMILES: c1ccc(C2CCCN2c2nc(Nc3cn(C4CCC4)cn3)c3cccn3n2)nc1]

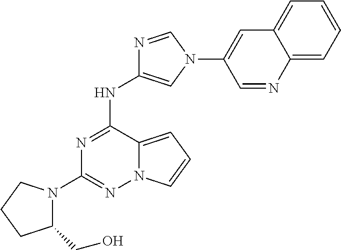 [SMILES: OC[C@@H]1CCCN1c1nc(Nc2cn(-c3cnc4ccccc4c3)cn2)c2cccn2n1]

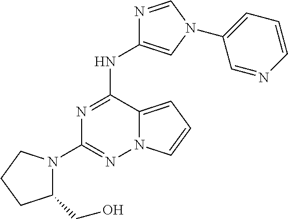 [SMILES: OC[C@@H]1CCCN1c1nc(Nc2cn(-c3cccnc3)cn2)c2cccn2n1]

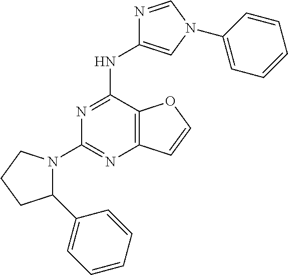 [SMILES: c1ccc(C2CCCN2c2nc(Nc3cn(-c4ccccc4)cn3)c3occc3n2)cc1]

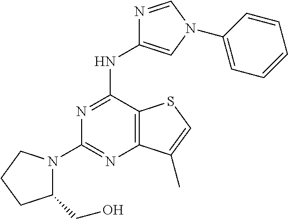 [SMILES: Cc1csc2c(Nc3cn(-c4ccccc4)cn3)nc(N3CCC[C@H]3CO)nc12]

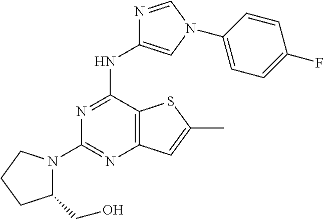 [SMILES: Cc1cc2nc(N3CCC[C@H]3CO)nc(Nc3cn(-c4ccc(F)cc4)cn3)c2s1]

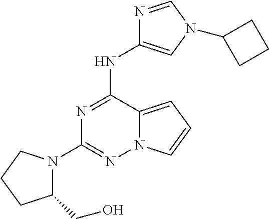 [SMILES: OC[C@@H]1CCCN1c1nc(Nc2cn(C3CCC3)cn2)c2cccn2n1]

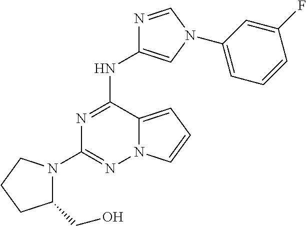 [SMILES: OC[C@@H]1CCCN1c1nc(Nc2cn(-c3cccc(F)c3)cn2)c2cccn2n1]

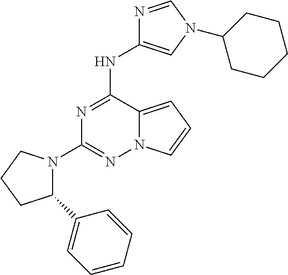 [SMILES: c1ccc([C@@H]2CCCN2c2nc(Nc3cn(C4CCCCC4)cn3)c3cccn3n2)cc1]

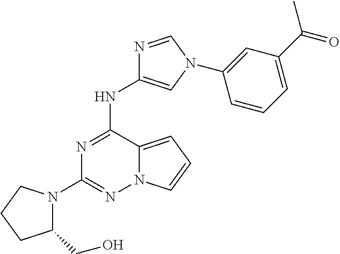 [SMILES: CC(=O)c1cccc(-n2cnc(Nc3nc(N4CCC[C@H]4CO)nn4cccc34)c2)c1]

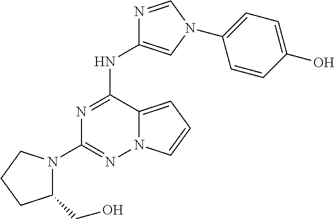 [SMILES: OC[C@@H]1CCCN1c1nc(Nc2cn(-c3ccc(O)cc3)cn2)c2cccn2n1]

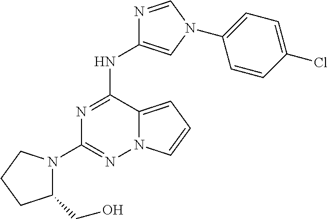 [SMILES: OC[C@@H]1CCCN1c1nc(Nc2cn(-c3ccc(Cl)cc3)cn2)c2cccn2n1]

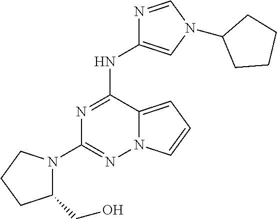 [SMILES: OC[C@@H]1CCCN1c1nc(Nc2cn(C3CCCC3)cn2)c2cccn2n1]

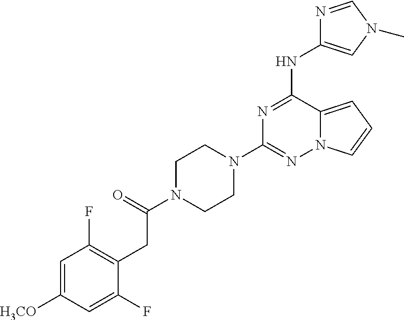 [SMILES: COc1cc(F)c(CC(=O)N2CCN(c3nc(Nc4cn(C)cn4)c4cccn4n3)CC2)c(F)c1]

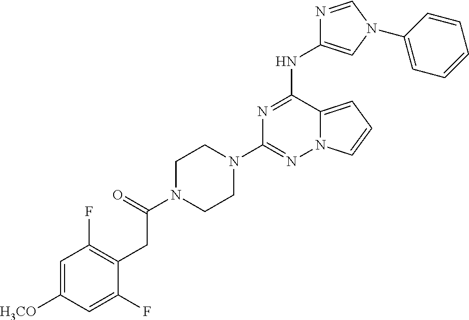 [SMILES: COc1cc(F)c(CC(=O)N2CCN(c3nc(Nc4cn(-c5ccccc5)cn4)c4cccn4n3)CC2)c(F)c1]

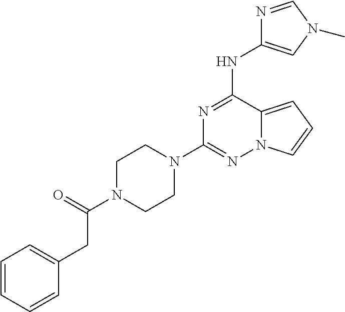 [SMILES: Cn1cnc(Nc2nc(N3CCN(C(=O)Cc4ccccc4)CC3)nn3cccc23)c1]

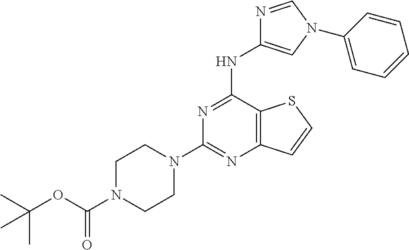 [SMILES: CC(C)(C)OC(=O)N1CCN(c2nc(Nc3cn(-c4ccccc4)cn3)c3sccc3n2)CC1]

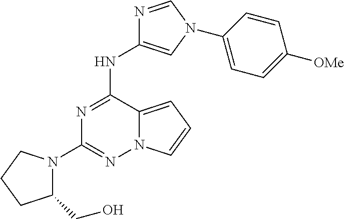 [SMILES: COc1ccc(-n2cnc(Nc3nc(N4CCC[C@H]4CO)nn4cccc34)c2)cc1]

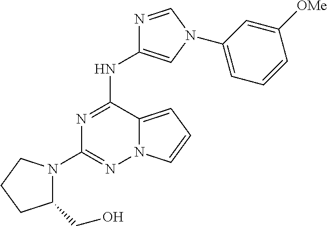 [SMILES: COc1cccc(-n2cnc(Nc3nc(N4CCC[C@H]4CO)nn4cccc34)c2)c1]

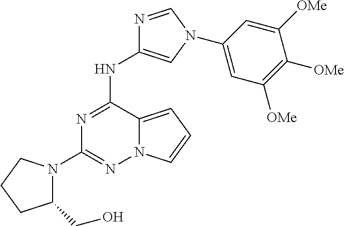 [SMILES: COc1cc(-n2cnc(Nc3nc(N4CCC[C@H]4CO)nn4cccc34)c2)cc(OC)c1OC]

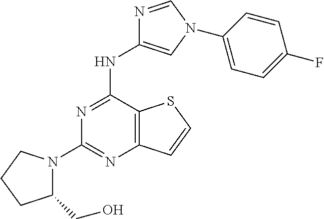 [SMILES: OC[C@@H]1CCCN1c1nc(Nc2cn(-c3ccc(F)cc3)cn2)c2sccc2n1]

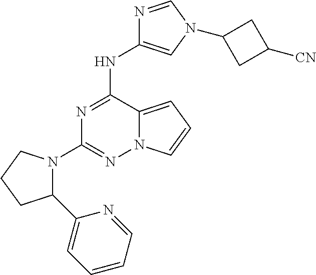 [SMILES: N#CC1CC(n2cnc(Nc3nc(N4CCCC4c4ccccn4)nn4cccc34)c2)C1]

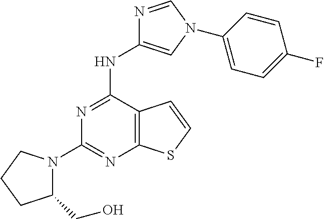 [SMILES: OC[C@@H]1CCCN1c1nc(Nc2cn(-c3ccc(F)cc3)cn2)c2ccsc2n1]

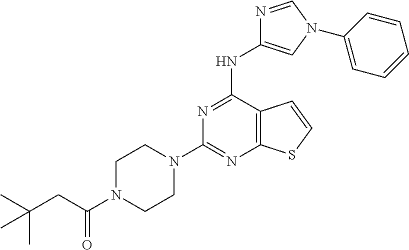 [SMILES: CC(C)(C)CC(=O)N1CCN(c2nc(Nc3cn(-c4ccccc4)cn3)c3ccsc3n2)CC1]

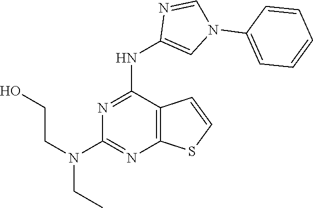 [SMILES: CCN(CCO)c1nc(Nc2cn(-c3ccccc3)cn2)c2ccsc2n1]

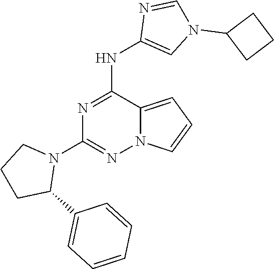 [SMILES: c1ccc([C@@H]2CCCN2c2nc(Nc3cn(C4CCC4)cn3)c3cccn3n2)cc1]

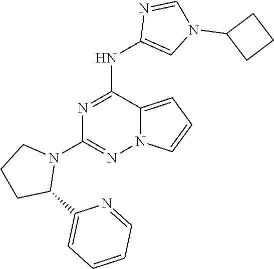 [SMILES: c1ccc([C@@H]2CCCN2c2nc(Nc3cn(C4CCC4)cn3)c3cccn3n2)nc1]

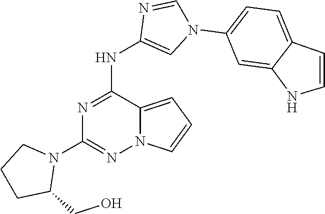 [SMILES: OC[C@@H]1CCCN1c1nc(Nc2cn(-c3ccc4cc[nH]c4c3)cn2)c2cccn2n1]

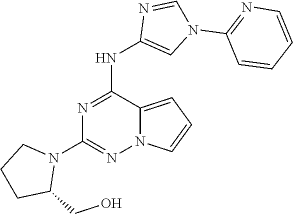 [SMILES: OC[C@@H]1CCCN1c1nc(Nc2cn(-c3ccccn3)cn2)c2cccn2n1]

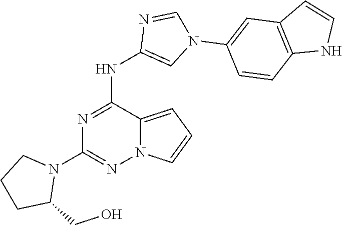 [SMILES: OC[C@@H]1CCCN1c1nc(Nc2cn(-c3ccc4[nH]ccc4c3)cn2)c2cccn2n1]

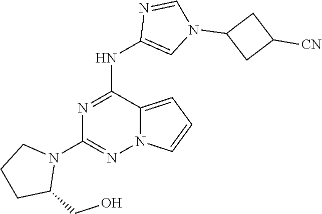 [SMILES: N#CC1CC(n2cnc(Nc3nc(N4CCC[C@H]4CO)nn4cccc34)c2)C1]

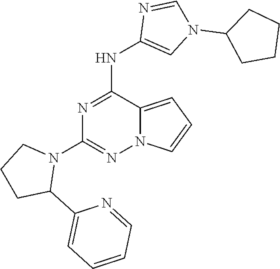 [SMILES: c1ccc(C2CCCN2c2nc(Nc3cn(C4CCCC4)cn3)c3cccn3n2)nc1]

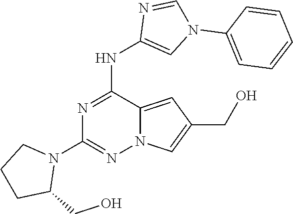 [SMILES: OCc1cc2c(Nc3cn(-c4ccccc4)cn3)nc(N3CCC[C@H]3CO)nn2c1]

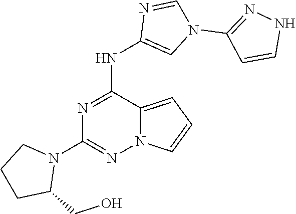 [SMILES: OC[C@@H]1CCCN1c1nc(Nc2cn(-c3cc[nH]n3)cn2)c2cccn2n1]

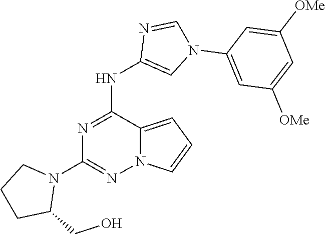 [SMILES: COc1cc(OC)cc(-n2cnc(Nc3nc(N4CCC[C@H]4CO)nn4cccc34)c2)c1]

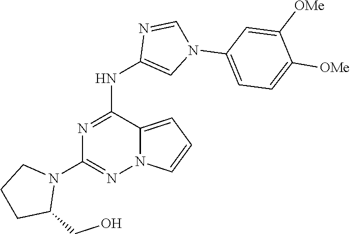 [SMILES: COc1ccc(-n2cnc(Nc3nc(N4CCC[C@H]4CO)nn4cccc34)c2)cc1OC]

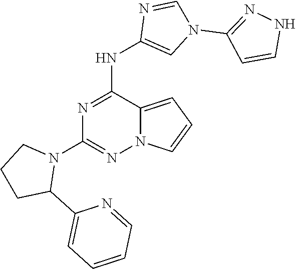 [SMILES: c1ccc(C2CCCN2c2nc(Nc3cn(-c4cc[nH]n4)cn3)c3cccn3n2)nc1]

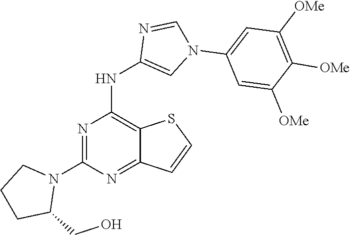 [SMILES: COc1cc(-n2cnc(Nc3nc(N4CCC[C@H]4CO)nc4ccsc34)c2)cc(OC)c1OC]

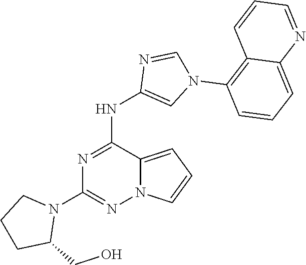 [SMILES: OC[C@@H]1CCCN1c1nc(Nc2cn(-c3cccc4ncccc34)cn2)c2cccn2n1]

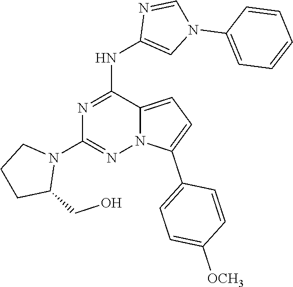 [SMILES: COc1ccc(-c2ccc3c(Nc4cn(-c5ccccc5)cn4)nc(N4CCC[C@H]4CO)nn23)cc1]

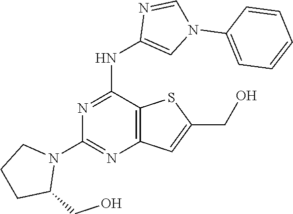 [SMILES: OCc1cc2nc(N3CCC[C@H]3CO)nc(Nc3cn(-c4ccccc4)cn3)c2s1]

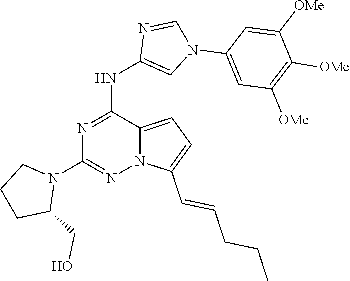 [SMILES: CCC/C=C/c1ccc2c(Nc3cn(-c4cc(OC)c(OC)c(OC)c4)cn3)nc(N3CCC[C@H]3CO)nn12]

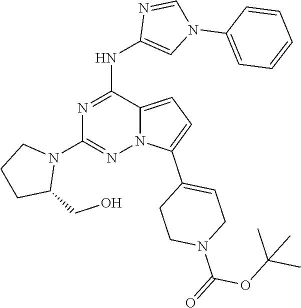 [SMILES: CC(C)(C)OC(=O)N1CC=C(c2ccc3c(Nc4cn(-c5ccccc5)cn4)nc(N4CCC[C@H]4CO)nn23)CC1]

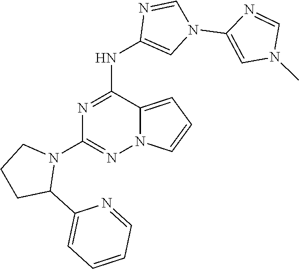 [SMILES: Cn1cnc(-n2cnc(Nc3nc(N4CCCC4c4ccccn4)nn4cccc34)c2)c1]

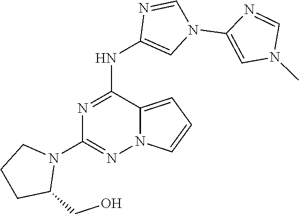 [SMILES: Cn1cnc(-n2cnc(Nc3nc(N4CCC[C@H]4CO)nn4cccc34)c2)c1]

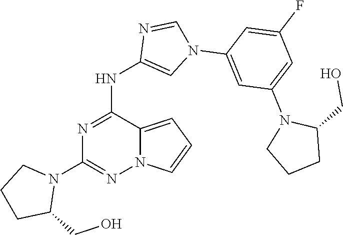 [SMILES: OC[C@@H]1CCCN1c1cc(F)cc(-n2cnc(Nc3nc(N4CCC[C@H]4CO)nn4cccc34)c2)c1]

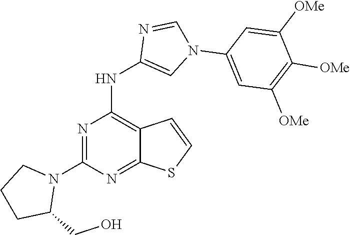 [SMILES: COc1cc(-n2cnc(Nc3nc(N4CCC[C@H]4CO)nc4sccc34)c2)cc(OC)c1OC]

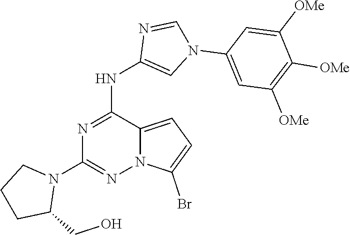 [SMILES: COc1cc(-n2cnc(Nc3nc(N4CCC[C@H]4CO)nn4c(Br)ccc34)c2)cc(OC)c1OC]

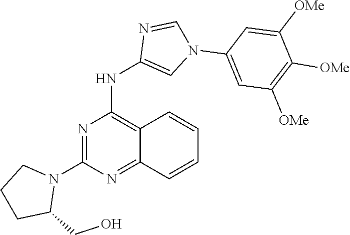 [SMILES: COc1cc(-n2cnc(Nc3nc(N4CCC[C@H]4CO)nc4ccccc34)c2)cc(OC)c1OC]

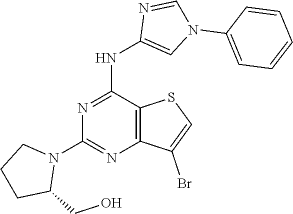 [SMILES: OC[C@@H]1CCCN1c1nc(Nc2cn(-c3ccccc3)cn2)c2scc(Br)c2n1]

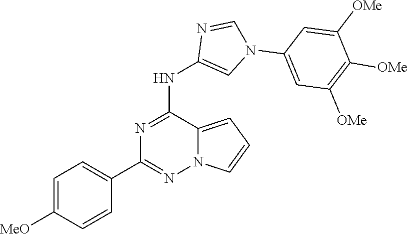 [SMILES: COc1ccc(-c2nc(Nc3cn(-c4cc(OC)c(OC)c(OC)c4)cn3)c3cccn3n2)cc1]

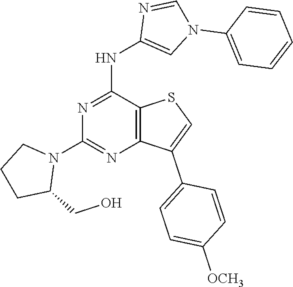 [SMILES: COc1ccc(-c2csc3c(Nc4cn(-c5ccccc5)cn4)nc(N4CCC[C@H]4CO)nc23)cc1]

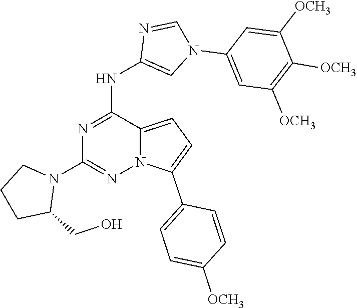 [SMILES: COc1ccc(-c2ccc3c(Nc4cn(-c5cc(OC)c(OC)c(OC)c5)cn4)nc(N4CCC[C@H]4CO)nn23)cc1]

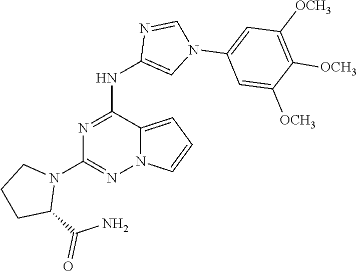 [SMILES: COc1cc(-n2cnc(Nc3nc(N4CCC[C@H]4C(N)=O)nn4cccc34)c2)cc(OC)c1OC]

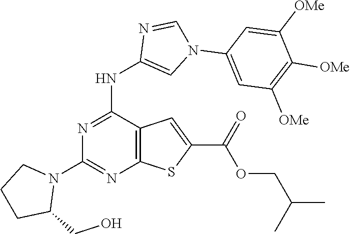 [SMILES: COc1cc(-n2cnc(Nc3nc(N4CCC[C@H]4CO)nc4sc(C(=O)OCC(C)C)cc34)c2)cc(OC)c1OC]

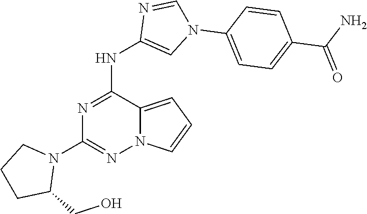 [SMILES: NC(=O)c1ccc(-n2cnc(Nc3nc(N4CCC[C@H]4CO)nn4cccc34)c2)cc1]

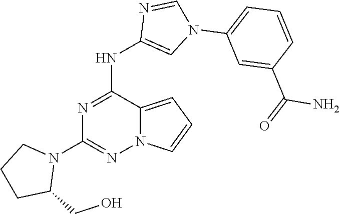 [SMILES: NC(=O)c1cccc(-n2cnc(Nc3nc(N4CCC[C@H]4CO)nn4cccc34)c2)c1]

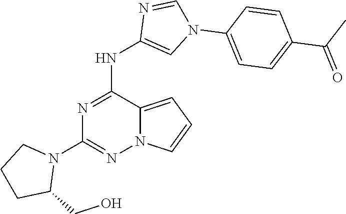 [SMILES: CC(=O)c1ccc(-n2cnc(Nc3nc(N4CCC[C@H]4CO)nn4cccc34)c2)cc1]

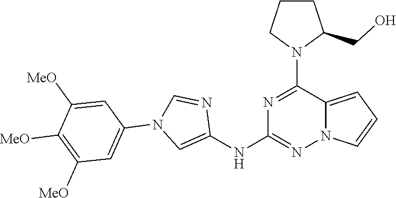 [SMILES: COc1cc(-n2cnc(Nc3nc(N4CCC[C@H]4CO)c4cccn4n3)c2)cc(OC)c1OC]

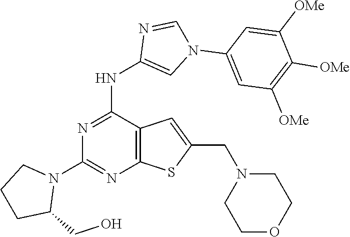 [SMILES: COc1cc(-n2cnc(Nc3nc(N4CCC[C@H]4CO)nc4sc(CN5CCOCC5)cc34)c2)cc(OC)c1OC]

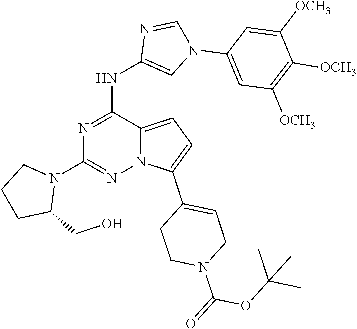 [SMILES: COc1cc(-n2cnc(Nc3nc(N4CCC[C@H]4CO)nn4c(C5=CCN(C(=O)OC(C)(C)C)CC5)ccc34)c2)cc(OC)c1OC]

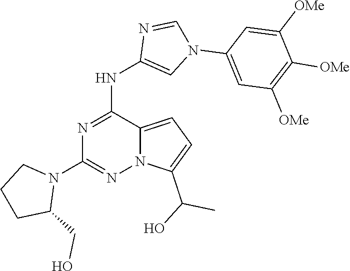 [SMILES: COc1cc(-n2cnc(Nc3nc(N4CCC[C@H]4CO)nn4c(C(C)O)ccc34)c2)cc(OC)c1OC]

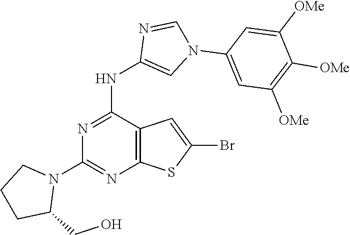 [SMILES: COc1cc(-n2cnc(Nc3nc(N4CCC[C@H]4CO)nc4sc(Br)cc34)c2)cc(OC)c1OC]

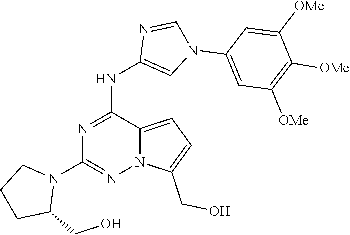 [SMILES: COc1cc(-n2cnc(Nc3nc(N4CCC[C@H]4CO)nn4c(CO)ccc34)c2)cc(OC)c1OC]

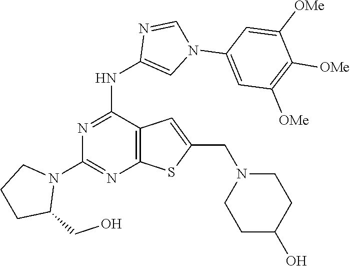 [SMILES: COc1cc(-n2cnc(Nc3nc(N4CCC[C@H]4CO)nc4sc(CN5CCC(O)CC5)cc34)c2)cc(OC)c1OC]

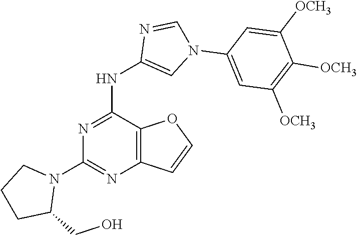 [SMILES: COc1cc(-n2cnc(Nc3nc(N4CCC[C@H]4CO)nc4ccoc34)c2)cc(OC)c1OC]

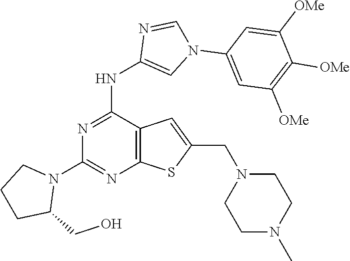 [SMILES: COc1cc(-n2cnc(Nc3nc(N4CCC[C@H]4CO)nc4sc(CN5CCN(C)CC5)cc34)c2)cc(OC)c1OC]

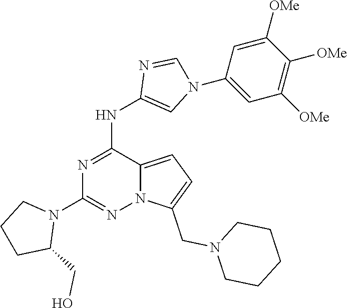 [SMILES: COc1cc(-n2cnc(Nc3nc(N4CCC[C@H]4CO)nn4c(CN5CCCCC5)ccc34)c2)cc(OC)c1OC]

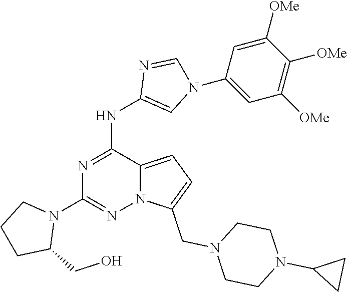 [SMILES: COc1cc(-n2cnc(Nc3nc(N4CCC[C@H]4CO)nn4c(CN5CCN(C6CC6)CC5)ccc34)c2)cc(OC)c1OC]

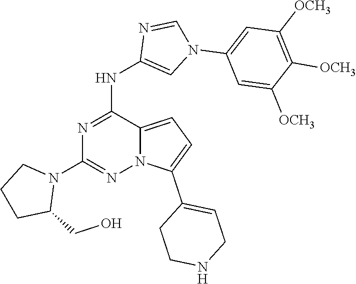 [SMILES: COc1cc(-n2cnc(Nc3nc(N4CCC[C@H]4CO)nn4c(C5=CCNCC5)ccc34)c2)cc(OC)c1OC]

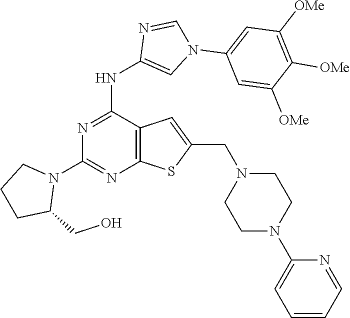 [SMILES: COc1cc(-n2cnc(Nc3nc(N4CCC[C@H]4CO)nc4sc(CN5CCN(c6ccccn6)CC5)cc34)c2)cc(OC)c1OC]

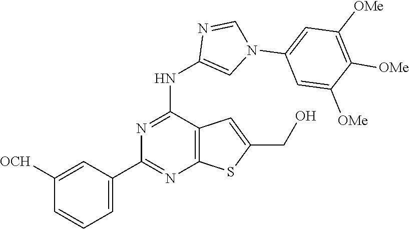

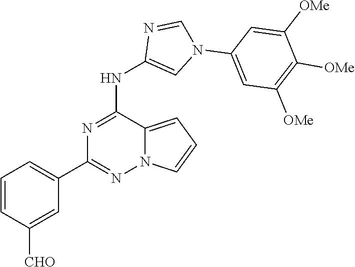 [SMILES: COc1cc(-n2cnc(Nc3nc(-c4cccc(C=O)c4)nn4cccc34)c2)cc(OC)c1OC]

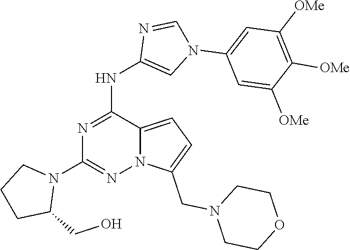 [SMILES: COc1cc(-n2cnc(Nc3nc(N4CCC[C@H]4CO)nn4c(CN5CCOCC5)ccc34)c2)cc(OC)c1OC]

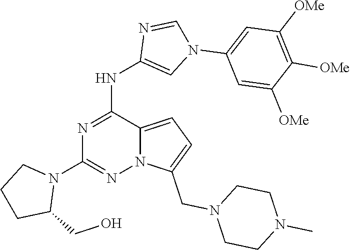 [SMILES: COc1cc(-n2cnc(Nc3nc(N4CCC[C@H]4CO)nn4c(CN5CCN(C)CC5)ccc34)c2)cc(OC)c1OC]

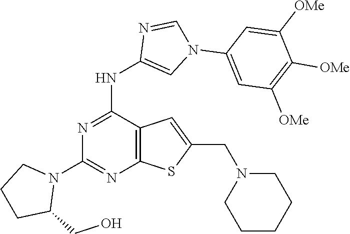 [SMILES: COc1cc(-n2cnc(Nc3nc(N4CCC[C@H]4CO)nc4sc(CN5CCCCC5)cc34)c2)cc(OC)c1OC]

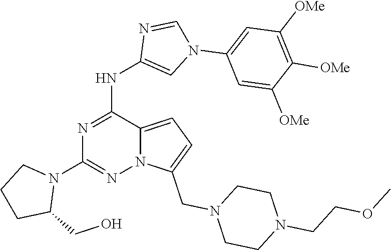 [SMILES: COCCN1CCN(Cc2ccc3c(Nc4cn(-c5cc(OC)c(OC)c(OC)c5)cn4)nc(N4CCC[C@H]4CO)nn23)CC1]

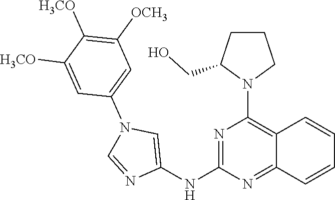 [SMILES: COc1cc(-n2cnc(Nc3nc(N4CCC[C@H]4CO)c4ccccc4n3)c2)cc(OC)c1OC]

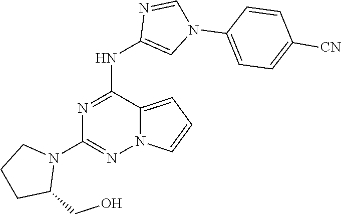 [SMILES: N#Cc1ccc(-n2cnc(Nc3nc(N4CCC[C@H]4CO)nn4cccc34)c2)cc1]

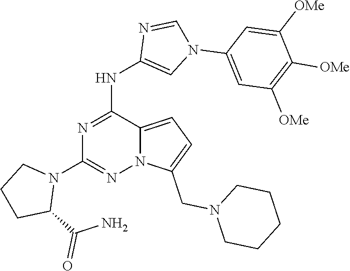 [SMILES: COc1cc(-n2cnc(Nc3nc(N4CCC[C@H]4C(N)=O)nn4c(CN5CCCCC5)ccc34)c2)cc(OC)c1OC]

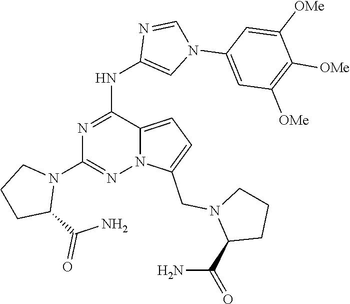 [SMILES: COc1cc(-n2cnc(Nc3nc(N4CCC[C@H]4C(N)=O)nn4c(CN5CCC[C@H]5C(N)=O)ccc34)c2)cc(OC)c1OC]

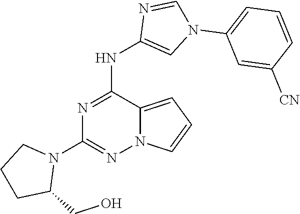 [SMILES: N#Cc1cccc(-n2cnc(Nc3nc(N4CCC[C@H]4CO)nn4cccc34)c2)c1]

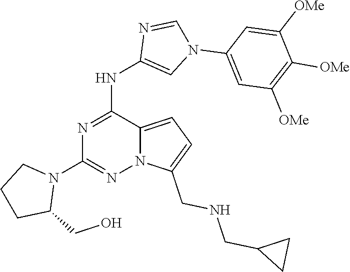 [SMILES: COc1cc(-n2cnc(Nc3nc(N4CCC[C@H]4CO)nn4c(CNCC5CC5)ccc34)c2)cc(OC)c1OC]

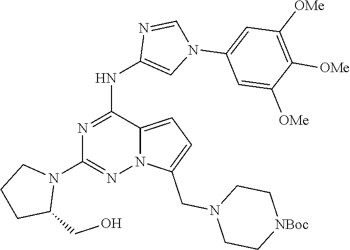 [SMILES: COc1cc(-n2cnc(Nc3nc(N4CCC[C@H]4CO)nn4c(CN5CCN(C(=O)OC(C)(C)C)CC5)ccc34)c2)cc(OC)c1OC]

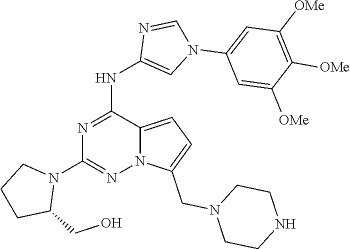 [SMILES: COc1cc(-n2cnc(Nc3nc(N4CCC[C@H]4CO)nn4c(CN5CCNCC5)ccc34)c2)cc(OC)c1OC]

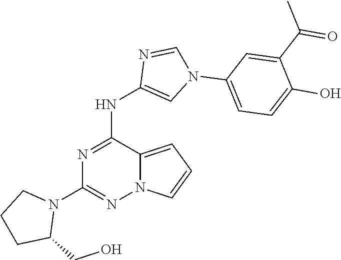 [SMILES: CC(=O)c1cc(-n2cnc(Nc3nc(N4CCC[C@H]4CO)nn4cccc34)c2)ccc1O]

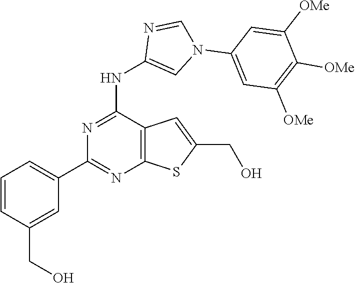 [SMILES: COc1cc(-n2cnc(Nc3nc(-c4cccc(CO)c4)nc4sc(CO)cc34)c2)cc(OC)c1OC]

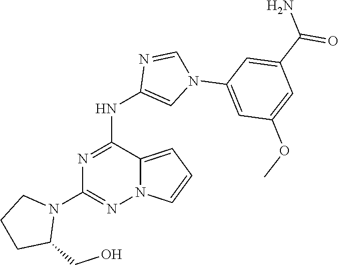 [SMILES: COc1cc(C(N)=O)cc(-n2cnc(Nc3nc(N4CCC[C@H]4CO)nn4cccc34)c2)c1]

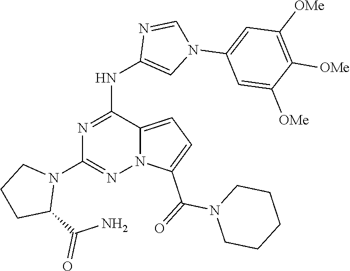 [SMILES: COc1cc(-n2cnc(Nc3nc(N4CCC[C@H]4C(N)=O)nn4c(C(=O)N5CCCCC5)ccc34)c2)cc(OC)c1OC]

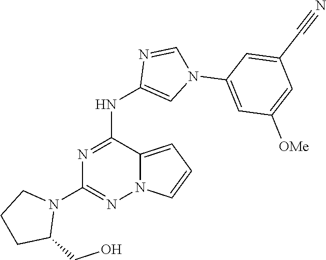 [SMILES: COc1cc(C#N)cc(-n2cnc(Nc3nc(N4CCC[C@H]4CO)nn4cccc34)c2)c1]

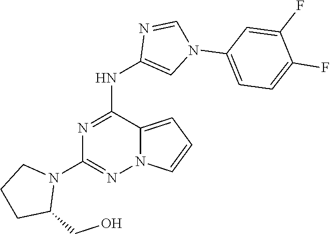 [SMILES: OC[C@@H]1CCCN1c1nc(Nc2cn(-c3ccc(F)c(F)c3)cn2)c2cccn2n1]

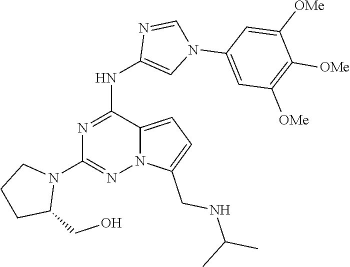 [SMILES: COc1cc(-n2cnc(Nc3nc(N4CCC[C@H]4CO)nn4c(CNC(C)C)ccc34)c2)cc(OC)c1OC]

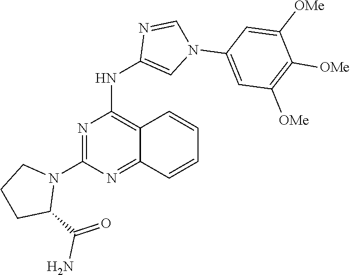 [SMILES: COc1cc(-n2cnc(Nc3nc(N4CCC[C@H]4C(N)=O)nc4ccccc34)c2)cc(OC)c1OC]

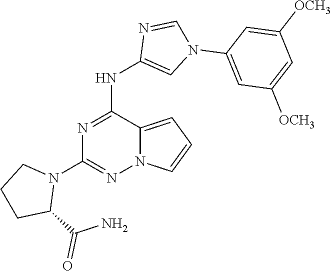 [SMILES: COc1cc(OC)cc(-n2cnc(Nc3nc(N4CCC[C@H]4C(N)=O)nn4cccc34)c2)c1]

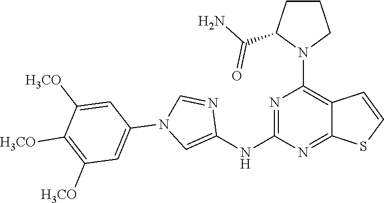 [SMILES: COc1cc(-n2cnc(Nc3nc(N4CCC[C@H]4C(N)=O)c4ccsc4n3)c2)cc(OC)c1OC]

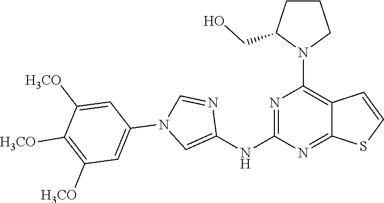 [SMILES: COc1cc(-n2cnc(Nc3nc(N4CCC[C@H]4CO)c4ccsc4n3)c2)cc(OC)c1OC]

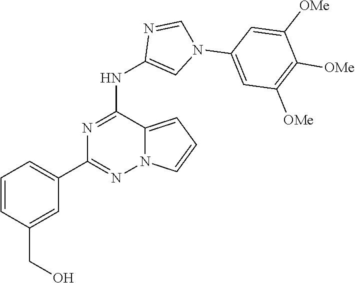 [SMILES: COc1cc(-n2cnc(Nc3nc(-c4cccc(CO)c4)nn4cccc34)c2)cc(OC)c1OC]

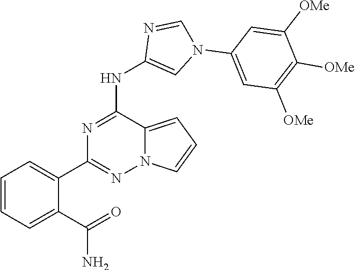 [SMILES: COc1cc(-n2cnc(Nc3nc(-c4ccccc4C(N)=O)nn4cccc34)c2)cc(OC)c1OC]

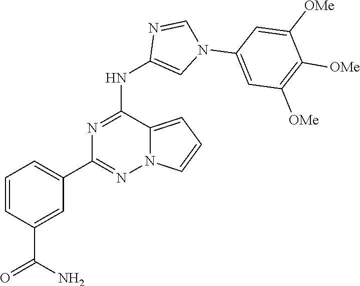 [SMILES: COc1cc(-n2cnc(Nc3nc(-c4cccc(C(N)=O)c4)nn4cccc34)c2)cc(OC)c1OC]

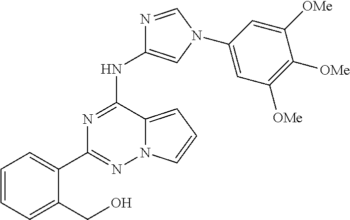 [SMILES: COc1cc(-n2cnc(Nc3nc(-c4ccccc4CO)nn4cccc34)c2)cc(OC)c1OC]

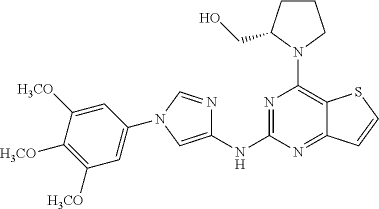 [SMILES: COc1cc(-n2cnc(Nc3nc(N4CCC[C@H]4CO)c4sccc4n3)c2)cc(OC)c1OC]

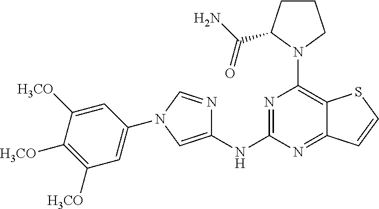 [SMILES: COc1cc(-n2cnc(Nc3nc(N4CCC[C@H]4C(N)=O)c4sccc4n3)c2)cc(OC)c1OC]

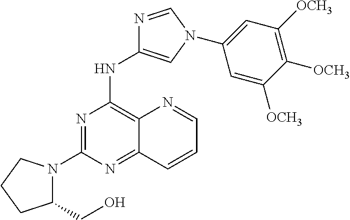 [SMILES: COc1cc(-n2cnc(Nc3nc(N4CCC[C@H]4CO)nc4cccnc34)c2)cc(OC)c1OC]

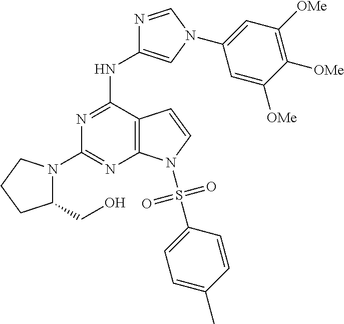 [SMILES: COc1cc(-n2cnc(Nc3nc(N4CCC[C@H]4CO)nc4c3ccn4S(=O)(=O)c3ccc(C)cc3)c2)cc(OC)c1OC]

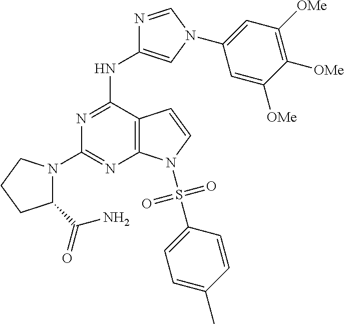 [SMILES: COc1cc(-n2cnc(Nc3nc(N4CCC[C@H]4C(N)=O)nc4c3ccn4S(=O)(=O)c3ccc(C)cc3)c2)cc(OC)c1OC]

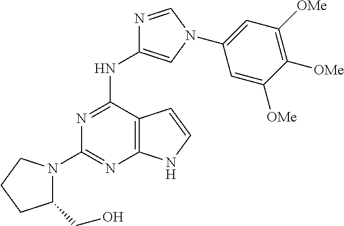 [SMILES: COc1cc(-n2cnc(Nc3nc(N4CCC[C@H]4CO)nc4[nH]ccc34)c2)cc(OC)c1OC]

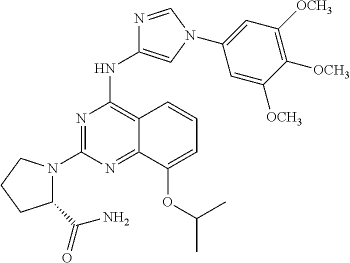 [SMILES: COc1cc(-n2cnc(Nc3nc(N4CCC[C@H]4C(N)=O)nc4c(OC(C)C)cccc34)c2)cc(OC)c1OC]

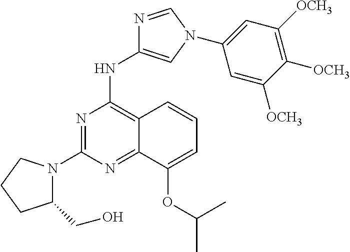 [SMILES: COc1cc(-n2cnc(Nc3nc(N4CCC[C@H]4CO)nc4c(OC(C)C)cccc34)c2)cc(OC)c1OC]

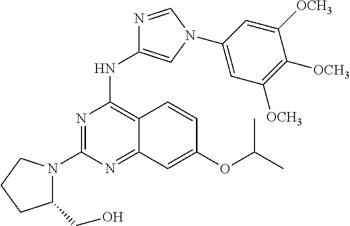 [SMILES: COc1cc(-n2cnc(Nc3nc(N4CCC[C@H]4CO)nc4cc(OC(C)C)ccc34)c2)cc(OC)c1OC]

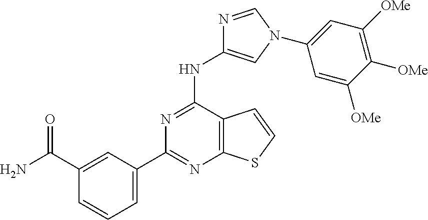 [SMILES: COc1cc(-n2cnc(Nc3nc(-c4cccc(C(N)=O)c4)nc4sccc34)c2)cc(OC)c1OC]

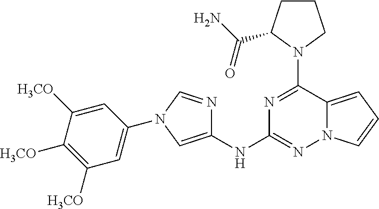 [SMILES: COc1cc(-n2cnc(Nc3nc(N4CCC[C@H]4C(N)=O)c4cccn4n3)c2)cc(OC)c1OC]

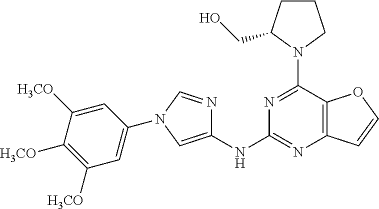 [SMILES: COc1cc(-n2cnc(Nc3nc(N4CCC[C@H]4CO)c4occc4n3)c2)cc(OC)c1OC]

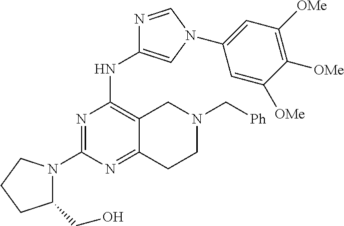 [SMILES: COc1cc(-n2cnc(Nc3nc(N4CCC[C@H]4CO)nc4c3CN(Cc3ccccc3)CC4)c2)cc(OC)c1OC]

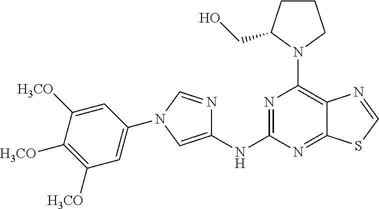 [SMILES: COc1cc(-n2cnc(Nc3nc(N4CCC[C@H]4CO)c4ncsc4n3)c2)cc(OC)c1OC]

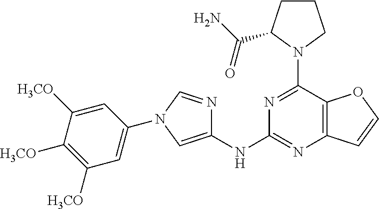 [SMILES: COc1cc(-n2cnc(Nc3nc(N4CCC[C@H]4C(N)=O)c4occc4n3)c2)cc(OC)c1OC]

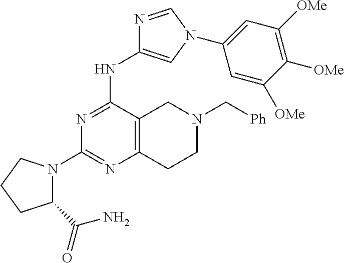 [SMILES: COc1cc(-n2cnc(Nc3nc(N4CCC[C@H]4C(N)=O)nc4c3CN(Cc3ccccc3)CC4)c2)cc(OC)c1OC]

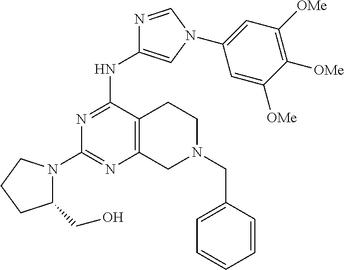 [SMILES: COc1cc(-n2cnc(Nc3nc(N4CCC[C@H]4CO)nc4c3CCN(Cc3ccccc3)C4)c2)cc(OC)c1OC]

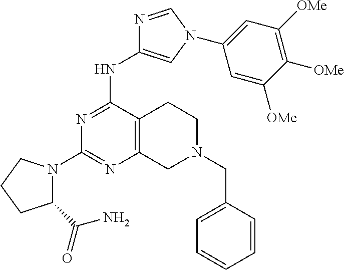 [SMILES: COc1cc(-n2cnc(Nc3nc(N4CCC[C@H]4C(N)=O)nc4c3CCN(Cc3ccccc3)C4)c2)cc(OC)c1OC]

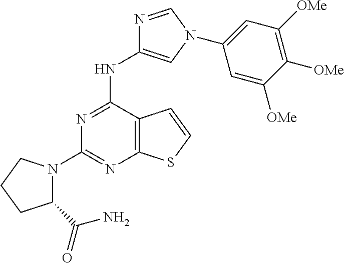 [SMILES: COc1cc(-n2cnc(Nc3nc(N4CCC[C@H]4C(N)=O)nc4sccc34)c2)cc(OC)c1OC]

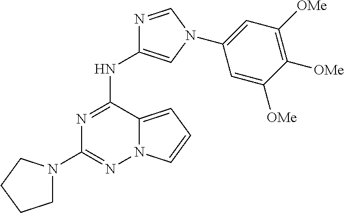 [SMILES: COc1cc(-n2cnc(Nc3nc(N4CCCC4)nn4cccc34)c2)cc(OC)c1OC]

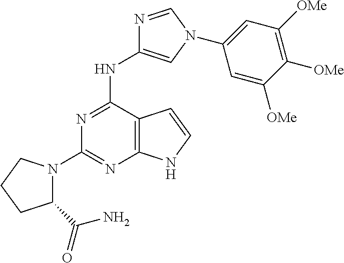 [SMILES: COc1cc(-n2cnc(Nc3nc(N4CCC[C@H]4C(N)=O)nc4[nH]ccc34)c2)cc(OC)c1OC]

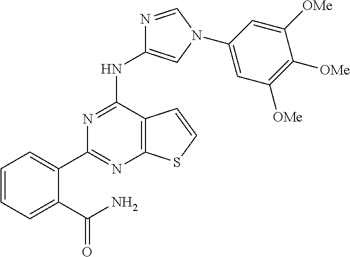 [SMILES: COc1cc(-n2cnc(Nc3nc(-c4ccccc4C(N)=O)nc4sccc34)c2)cc(OC)c1OC]

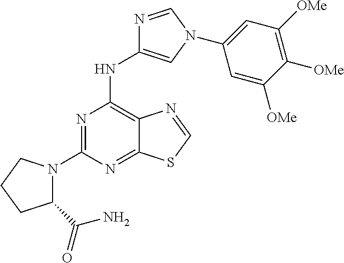 [SMILES: COc1cc(-n2cnc(Nc3nc(N4CCC[C@H]4C(N)=O)nc4scnc34)c2)cc(OC)c1OC]

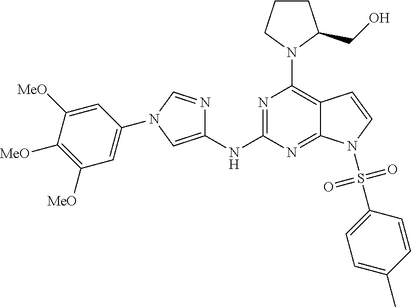 [SMILES: COc1cc(-n2cnc(Nc3nc(N4CCC[C@H]4CO)c4ccn(S(=O)(=O)c5ccc(C)cc5)c4n3)c2)cc(OC)c1OC]

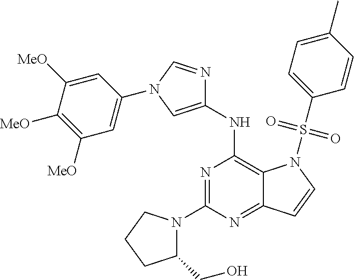 [SMILES: COc1cc(-n2cnc(Nc3nc(N4CCC[C@H]4CO)nc4ccn(S(=O)(=O)c5ccc(C)cc5)c34)c2)cc(OC)c1OC]

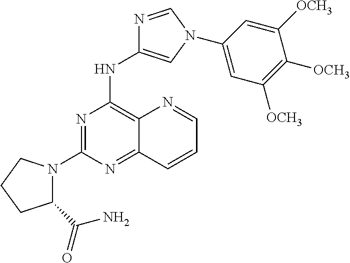 [SMILES: COc1cc(-n2cnc(Nc3nc(N4CCC[C@H]4C(N)=O)nc4cccnc34)c2)cc(OC)c1OC]

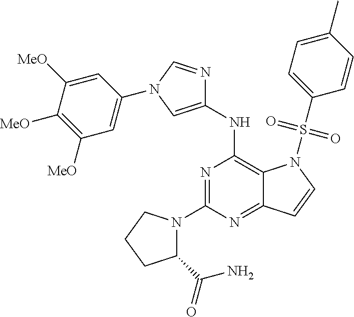 [SMILES: COc1cc(-n2cnc(Nc3nc(N4CCC[C@H]4C(N)=O)nc4ccn(S(=O)(=O)c5ccc(C)cc5)c34)c2)cc(OC)c1OC]

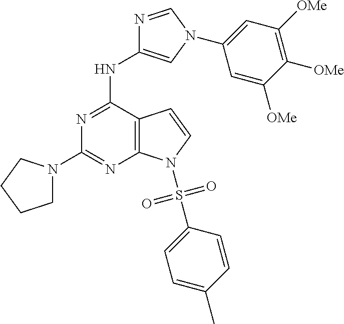 [SMILES: COc1cc(-n2cnc(Nc3nc(N4CCCC4)nc4c3ccn4S(=O)(=O)c3ccc(C)cc3)c2)cc(OC)c1OC]

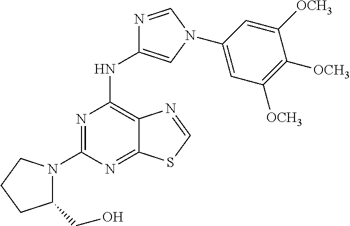 [SMILES: COc1cc(-n2cnc(Nc3nc(N4CCC[C@H]4CO)nc4scnc34)c2)cc(OC)c1OC]

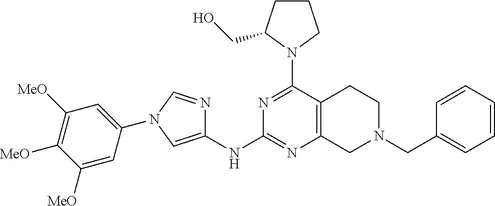 [SMILES: COc1cc(-n2cnc(Nc3nc4c(c(N5CCC[C@H]5CO)n3)CCN(Cc3ccccc3)C4)c2)cc(OC)c1OC]

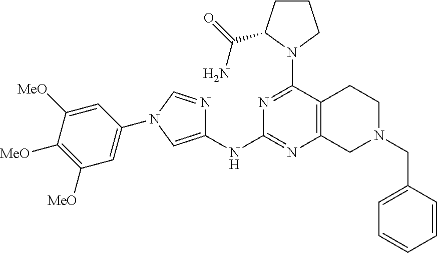 [SMILES: COc1cc(-n2cnc(Nc3nc4c(c(N5CCC[C@H]5C(N)=O)n3)CCN(Cc3ccccc3)C4)c2)cc(OC)c1OC]

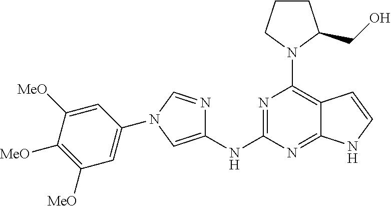 [SMILES: COc1cc(-n2cnc(Nc3nc(N4CCC[C@H]4CO)c4cc[nH]c4n3)c2)cc(OC)c1OC]

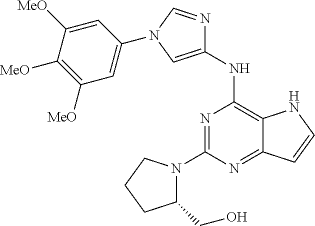 [SMILES: COc1cc(-n2cnc(Nc3nc(N4CCC[C@H]4CO)nc4cc[nH]c34)c2)cc(OC)c1OC]

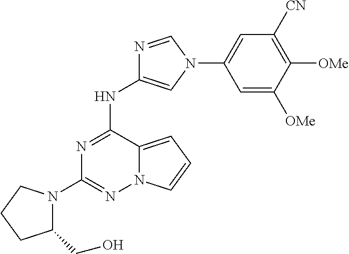 [SMILES: COc1cc(-n2cnc(Nc3nc(N4CCC[C@H]4CO)nn4cccc34)c2)cc(C#N)c1OC]

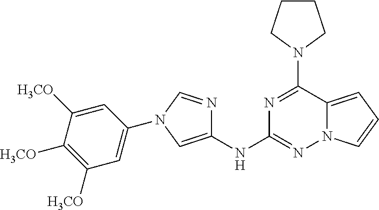 [SMILES: COc1cc(-n2cnc(Nc3nc(N4CCCC4)c4cccn4n3)c2)cc(OC)c1OC]

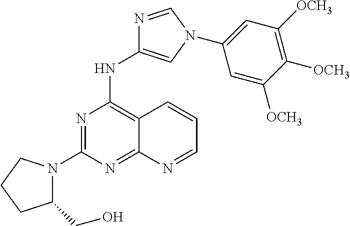 [SMILES: COc1cc(-n2cnc(Nc3nc(N4CCC[C@H]4CO)nc4ncccc34)c2)cc(OC)c1OC]

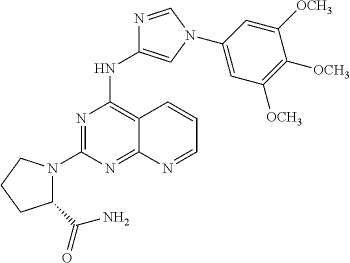 [SMILES: COc1cc(-n2cnc(Nc3nc(N4CCC[C@H]4C(N)=O)nc4ncccc34)c2)cc(OC)c1OC]

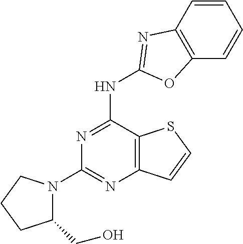 [SMILES: OC[C@@H]1CCCN1c1nc(Nc2nc3ccccc3o2)c2sccc2n1]

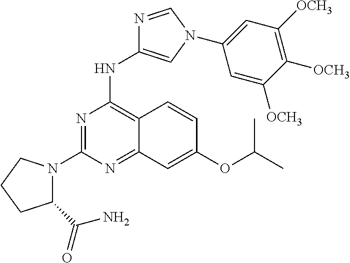 [SMILES: COc1cc(-n2cnc(Nc3nc(N4CCC[C@H]4C(N)=O)nc4cc(OC(C)C)ccc34)c2)cc(OC)c1OC]

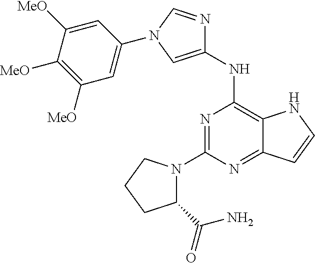 [SMILES: COc1cc(-n2cnc(Nc3nc(N4CCC[C@H]4C(N)=O)nc4cc[nH]c34)c2)cc(OC)c1OC]

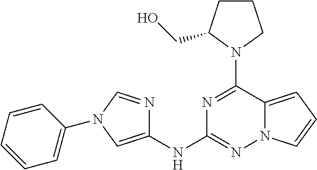 [SMILES: OC[C@@H]1CCCN1c1nc(Nc2cn(-c3ccccc3)cn2)nn2cccc12]

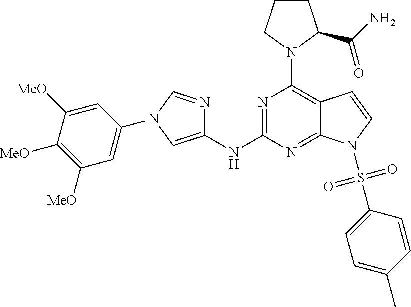 [SMILES: COc1cc(-n2cnc(Nc3nc(N4CCC[C@H]4C(N)=O)c4ccn(S(=O)(=O)c5ccc(C)cc5)c4n3)c2)cc(OC)c1OC]

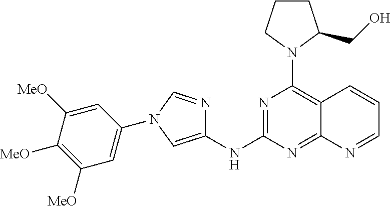 [SMILES: COc1cc(-n2cnc(Nc3nc(N4CCC[C@H]4CO)c4cccnc4n3)c2)cc(OC)c1OC]

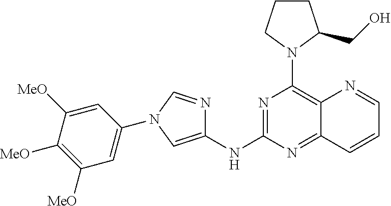 [SMILES: COc1cc(-n2cnc(Nc3nc(N4CCC[C@H]4CO)c4ncccc4n3)c2)cc(OC)c1OC]

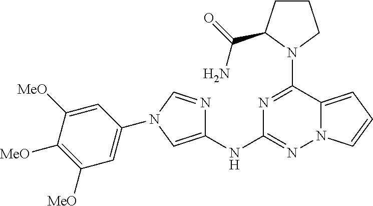 [SMILES: COc1cc(-n2cnc(Nc3nc(N4CCC[C@@H]4C(N)=O)c4cccn4n3)c2)cc(OC)c1OC]

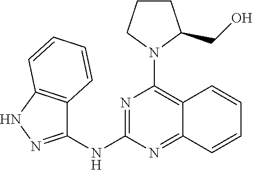 [SMILES: OC[C@@H]1CCCN1c1nc(Nc2n[nH]c3ccccc23)nc2ccccc12]

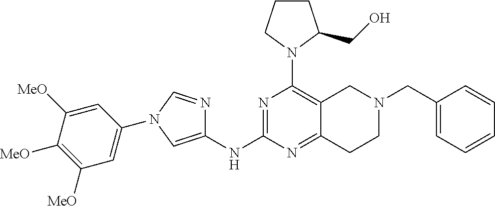 [SMILES: COc1cc(-n2cnc(Nc3nc4c(c(N5CCC[C@H]5CO)n3)CN(Cc3ccccc3)CC4)c2)cc(OC)c1OC]

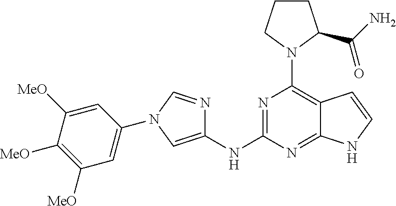 [SMILES: COc1cc(-n2cnc(Nc3nc(N4CCC[C@H]4C(N)=O)c4cc[nH]c4n3)c2)cc(OC)c1OC]

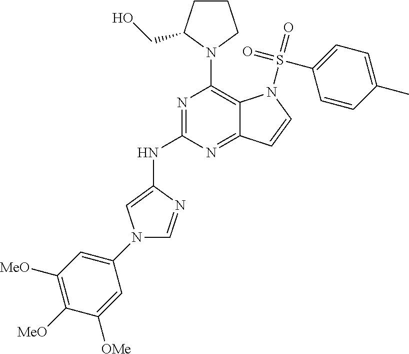 [SMILES: COc1cc(-n2cnc(Nc3nc(N4CCC[C@H]4CO)c4c(ccn4S(=O)(=O)c4ccc(C)cc4)n3)c2)cc(OC)c1OC]

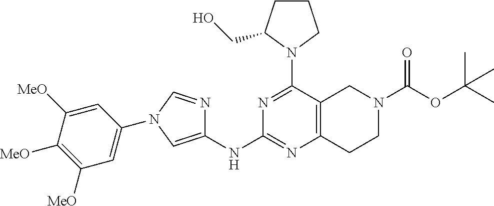 [SMILES: COc1cc(-n2cnc(Nc3nc4c(c(N5CCC[C@H]5CO)n3)CN(C(=O)OC(C)(C)C)CC4)c2)cc(OC)c1OC]

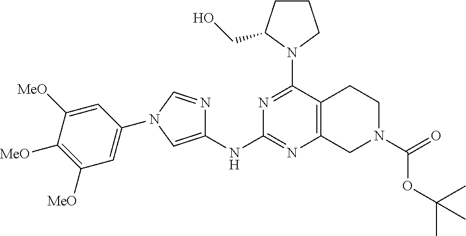 [SMILES: COc1cc(-n2cnc(Nc3nc4c(c(N5CCC[C@H]5CO)n3)CCN(C(=O)OC(C)(C)C)C4)c2)cc(OC)c1OC]

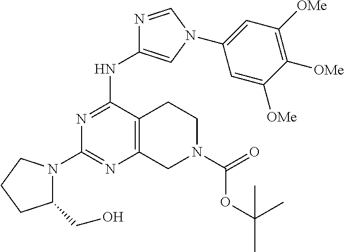 [SMILES: COc1cc(-n2cnc(Nc3nc(N4CCC[C@H]4CO)nc4c3CCN(C(=O)OC(C)(C)C)C4)c2)cc(OC)c1OC]

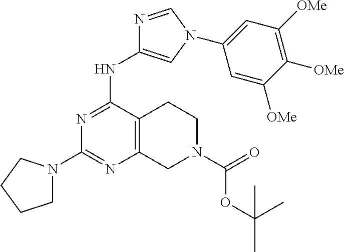 [SMILES: COc1cc(-n2cnc(Nc3nc(N4CCCC4)nc4c3CCN(C(=O)OC(C)(C)C)C4)c2)cc(OC)c1OC]

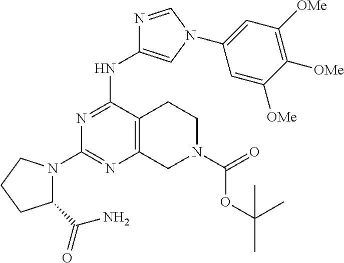 [SMILES: COc1cc(-n2cnc(Nc3nc(N4CCC[C@H]4C(N)=O)nc4c3CCN(C(=O)OC(C)(C)C)C4)c2)cc(OC)c1OC]

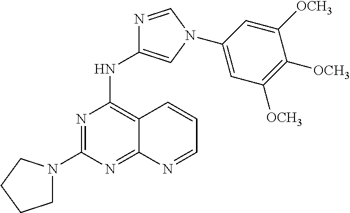 [SMILES: COc1cc(-n2cnc(Nc3nc(N4CCCC4)nc4ncccc34)c2)cc(OC)c1OC]

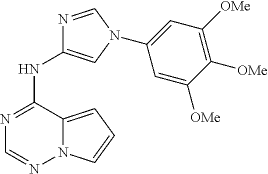 [SMILES: COc1cc(-n2cnc(Nc3ncnn4cccc34)c2)cc(OC)c1OC]

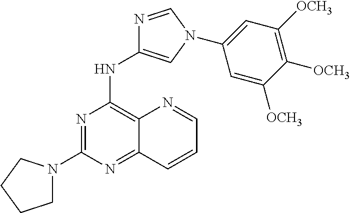 [SMILES: COc1cc(-n2cnc(Nc3nc(N4CCCC4)nc4cccnc34)c2)cc(OC)c1OC]

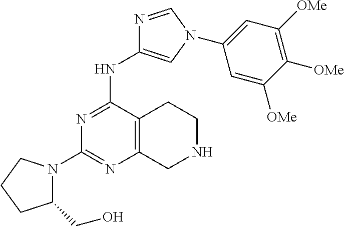 [SMILES: COc1cc(-n2cnc(Nc3nc(N4CCC[C@H]4CO)nc4c3CCNC4)c2)cc(OC)c1OC]

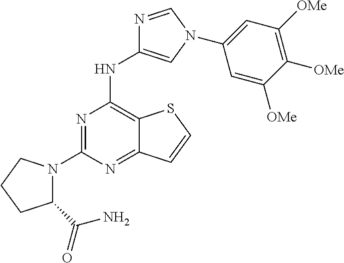 [SMILES: COc1cc(-n2cnc(Nc3nc(N4CCC[C@H]4C(N)=O)nc4ccsc34)c2)cc(OC)c1OC]

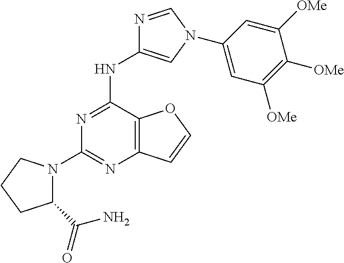 [SMILES: COc1cc(-n2cnc(Nc3nc(N4CCC[C@H]4C(N)=O)nc4ccoc34)c2)cc(OC)c1OC]

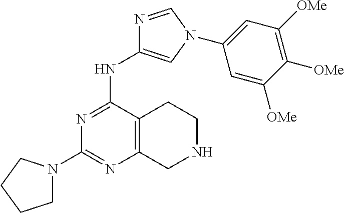 [SMILES: COc1cc(-n2cnc(Nc3nc(N4CCCC4)nc4c3CCNC4)c2)cc(OC)c1OC]

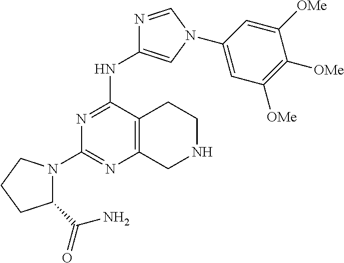 [SMILES: COc1cc(-n2cnc(Nc3nc(N4CCC[C@H]4C(N)=O)nc4c3CCNC4)c2)cc(OC)c1OC]

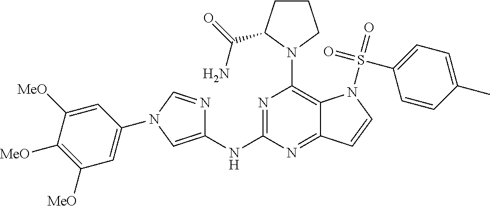 [SMILES: COc1cc(-n2cnc(Nc3nc(N4CCC[C@H]4C(N)=O)c4c(ccn4S(=O)(=O)c4ccc(C)cc4)n3)c2)cc(OC)c1OC]

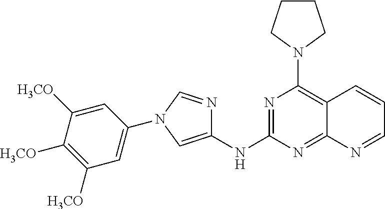 [SMILES: COc1cc(-n2cnc(Nc3nc(N4CCCC4)c4cccnc4n3)c2)cc(OC)c1OC]

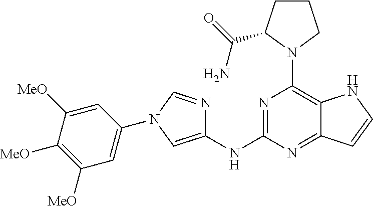 [SMILES: COc1cc(-n2cnc(Nc3nc(N4CCC[C@H]4C(N)=O)c4[nH]ccc4n3)c2)cc(OC)c1OC]

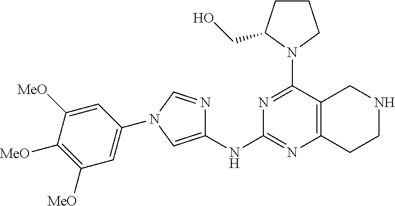 [SMILES: COc1cc(-n2cnc(Nc3nc4c(c(N5CCC[C@H]5CO)n3)CNCC4)c2)cc(OC)c1OC]

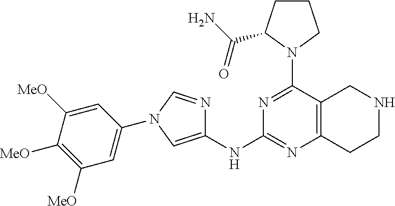 [SMILES: COc1cc(-n2cnc(Nc3nc4c(c(N5CCC[C@H]5C(N)=O)n3)CNCC4)c2)cc(OC)c1OC]

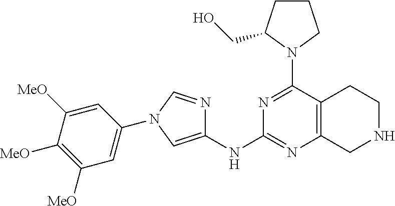 [SMILES: COc1cc(-n2cnc(Nc3nc4c(c(N5CCC[C@H]5CO)n3)CCNC4)c2)cc(OC)c1OC]

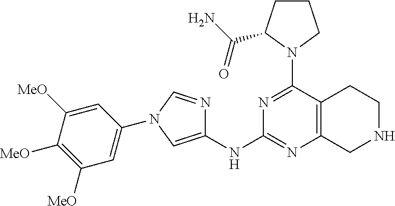 [SMILES: COc1cc(-n2cnc(Nc3nc4c(c(N5CCC[C@H]5C(N)=O)n3)CCNC4)c2)cc(OC)c1OC]

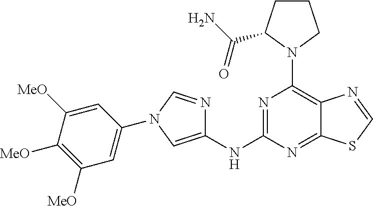 [SMILES: COc1cc(-n2cnc(Nc3nc(N4CCC[C@H]4C(N)=O)c4ncsc4n3)c2)cc(OC)c1OC]

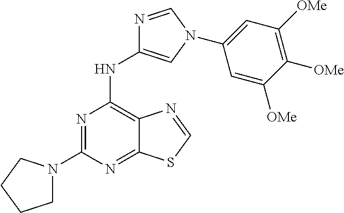 [SMILES: COc1cc(-n2cnc(Nc3nc(N4CCCC4)nc4scnc34)c2)cc(OC)c1OC]

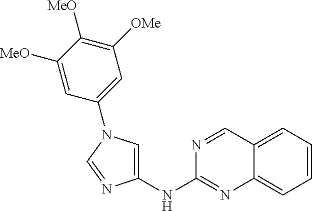 [SMILES: COc1cc(-n2cnc(Nc3ncc4ccccc4n3)c2)cc(OC)c1OC]

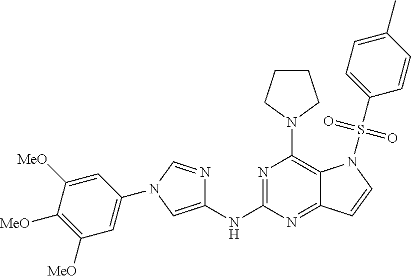 [SMILES: COc1cc(-n2cnc(Nc3nc(N4CCCC4)c4c(ccn4S(=O)(=O)c4ccc(C)cc4)n3)c2)cc(OC)c1OC]

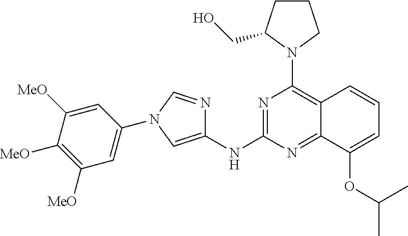 [SMILES: COc1cc(-n2cnc(Nc3nc(N4CCC[C@H]4CO)c4cccc(OC(C)C)c4n3)c2)cc(OC)c1OC]

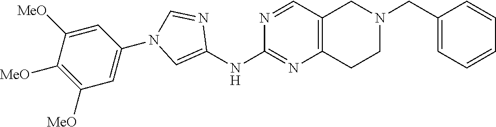 [SMILES: COc1cc(-n2cnc(Nc3ncc4c(n3)CCN(Cc3ccccc3)C4)c2)cc(OC)c1OC]

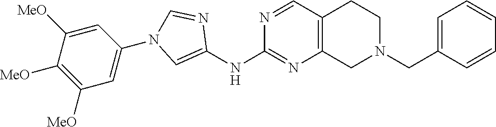 [SMILES: COc1cc(-n2cnc(Nc3ncc4c(n3)CN(Cc3ccccc3)CC4)c2)cc(OC)c1OC]

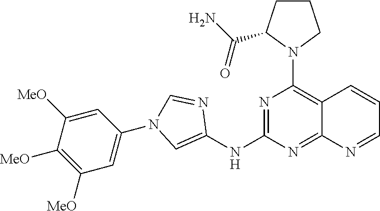 [SMILES: COc1cc(-n2cnc(Nc3nc(N4CCC[C@H]4C(N)=O)c4cccnc4n3)c2)cc(OC)c1OC]

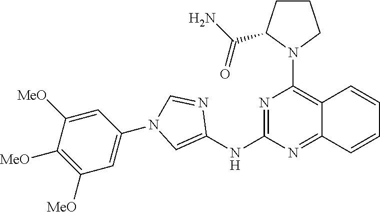 [SMILES: COc1cc(-n2cnc(Nc3nc(N4CCC[C@H]4C(N)=O)c4ccccc4n3)c2)cc(OC)c1OC]

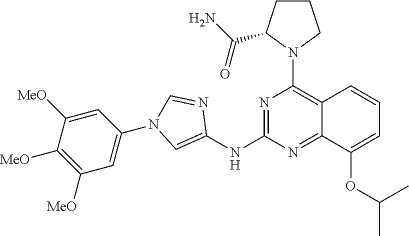 [SMILES: COc1cc(-n2cnc(Nc3nc(N4CCC[C@H]4C(N)=O)c4cccc(OC(C)C)c4n3)c2)cc(OC)c1OC]

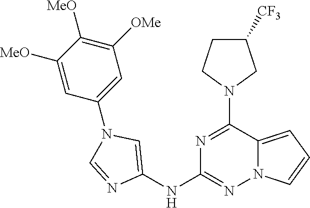 [SMILES: COc1cc(-n2cnc(Nc3nc(N4CC[C@H](C(F)(F)F)C4)c4cccn4n3)c2)cc(OC)c1OC]

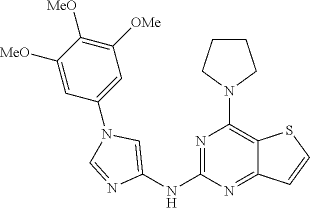 [SMILES: COc1cc(-n2cnc(Nc3nc(N4CCCC4)c4sccc4n3)c2)cc(OC)c1OC]

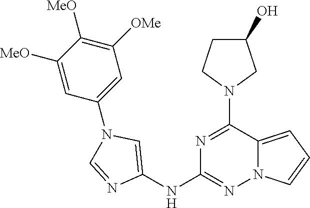 [SMILES: COc1cc(-n2cnc(Nc3nc(N4CC[C@@H](O)C4)c4cccn4n3)c2)cc(OC)c1OC]

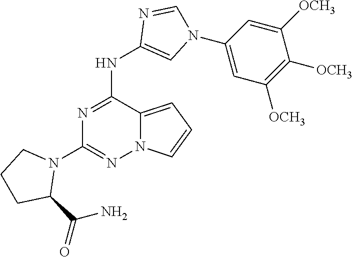 [SMILES: COc1cc(-n2cnc(Nc3nc(N4CCC[C@@H]4C(N)=O)nn4cccc34)c2)cc(OC)c1OC]

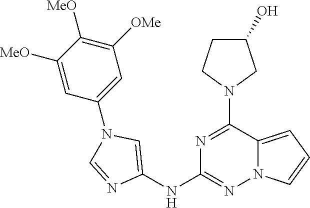 [SMILES: COc1cc(-n2cnc(Nc3nc(N4CC[C@H](O)C4)c4cccn4n3)c2)cc(OC)c1OC]

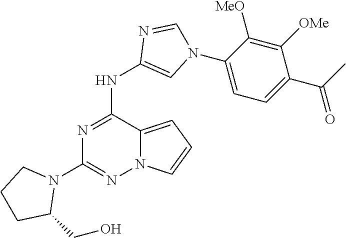 [SMILES: COc1c(C(C)=O)ccc(-n2cnc(Nc3nc(N4CCC[C@H]4CO)nn4cccc34)c2)c1OC]

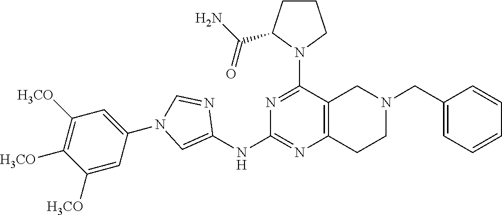 [SMILES: COc1cc(-n2cnc(Nc3nc4c(c(N5CCC[C@H]5C(N)=O)n3)CN(Cc3ccccc3)CC4)c2)cc(OC)c1OC]

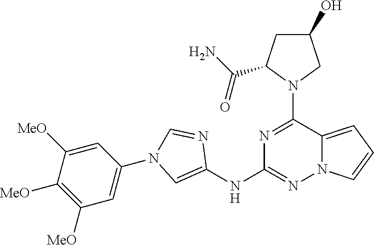 [SMILES: COc1cc(-n2cnc(Nc3nc(N4C[C@H](O)C[C@H]4C(N)=O)c4cccn4n3)c2)cc(OC)c1OC]

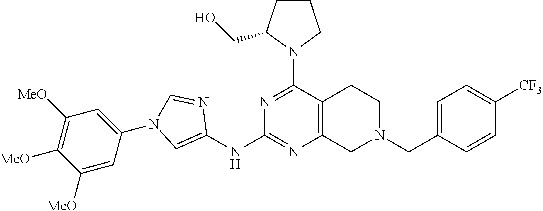

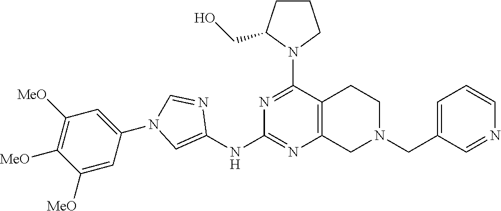

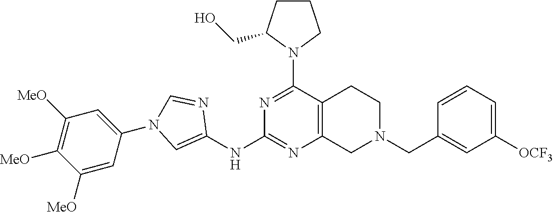

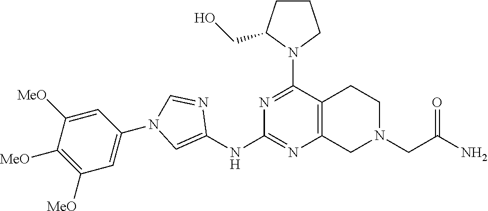

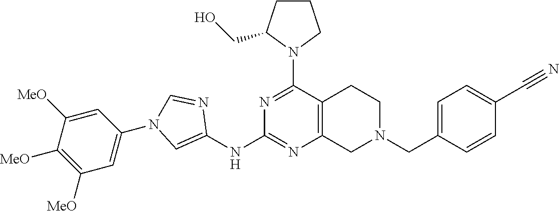

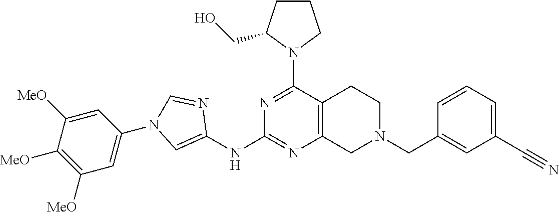

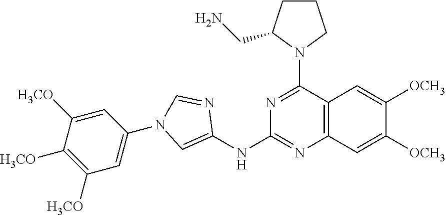 [SMILES: COc1cc2nc(Nc3cn(-c4cc(OC)c(OC)c(OC)c4)cn3)nc(N3CCC[C@H]3CN)c2cc1OC]

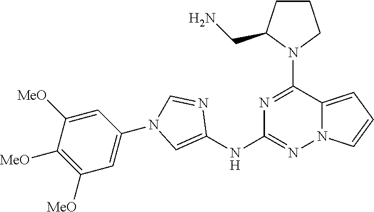 [SMILES: COc1cc(-n2cnc(Nc3nc(N4CCC[C@@H]4CN)c4cccn4n3)c2)cc(OC)c1OC]

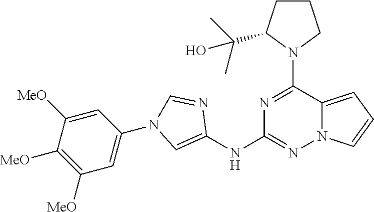 [SMILES: COc1cc(-n2cnc(Nc3nc(N4CCC[C@H]4C(C)(C)O)c4cccn4n3)c2)cc(OC)c1OC]

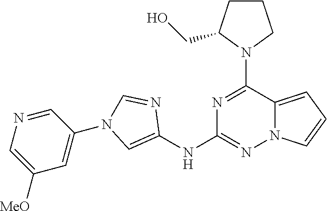 [SMILES: COc1cncc(-n2cnc(Nc3nc(N4CCC[C@H]4CO)c4cccn4n3)c2)c1]

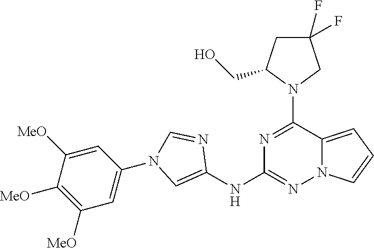 [SMILES: COc1cc(-n2cnc(Nc3nc(N4CC(F)(F)C[C@H]4CO)c4cccn4n3)c2)cc(OC)c1OC]

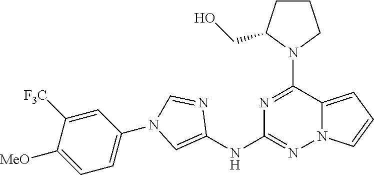 [SMILES: COc1ccc(-n2cnc(Nc3nc(N4CCC[C@H]4CO)c4cccn4n3)c2)cc1C(F)(F)F]

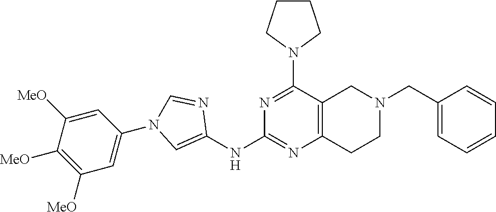

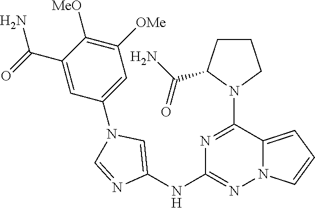 [SMILES: COc1cc(-n2cnc(Nc3nc(N4CCC[C@H]4C(N)=O)c4cccn4n3)c2)cc(C(N)=O)c1OC]

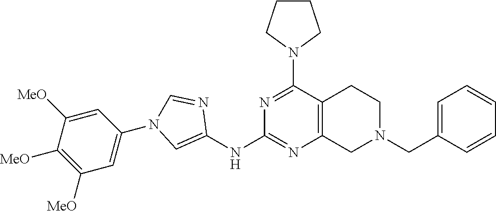

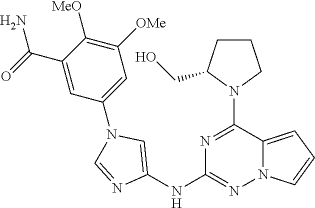 [SMILES: COc1cc(-n2cnc(Nc3nc(N4CCC[C@H]4CO)c4cccn4n3)c2)cc(C(N)=O)c1OC]

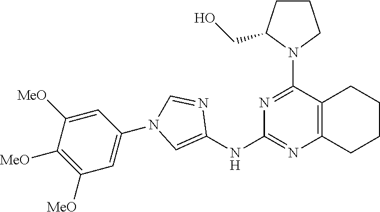 [SMILES: COc1cc(-n2cnc(Nc3nc4c(c(N5CCC[C@H]5CO)n3)CCCC4)c2)cc(OC)c1OC]

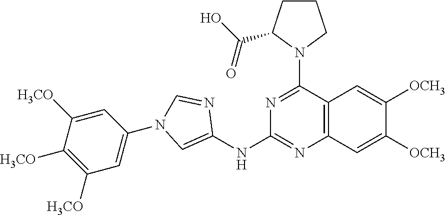 [SMILES: COc1cc2nc(Nc3cn(-c4cc(OC)c(OC)c(OC)c4)cn3)nc(N3CCC[C@H]3C(=O)O)c2cc1OC]

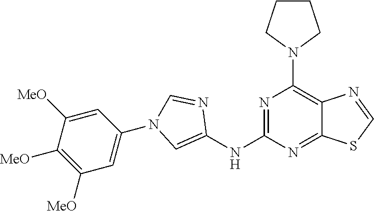 [SMILES: COc1cc(-n2cnc(Nc3nc(N4CCCC4)c4ncsc4n3)c2)cc(OC)c1OC]

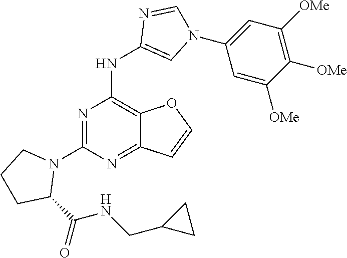 [SMILES: COc1cc(-n2cnc(Nc3nc(N4CCC[C@H]4C(=O)NCC4CC4)nc4ccoc34)c2)cc(OC)c1OC]

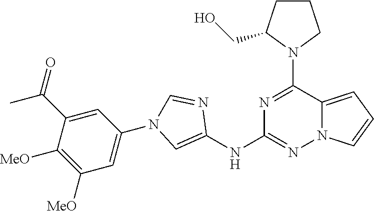 [SMILES: COc1cc(-n2cnc(Nc3nc(N4CCC[C@H]4CO)c4cccn4n3)c2)cc(C(C)=O)c1OC]

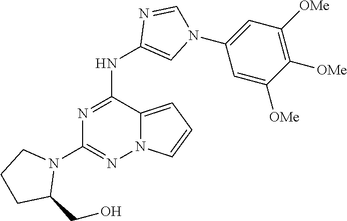 [SMILES: COc1cc(-n2cnc(Nc3nc(N4CCC[C@@H]4CO)nn4cccc34)c2)cc(OC)c1OC]

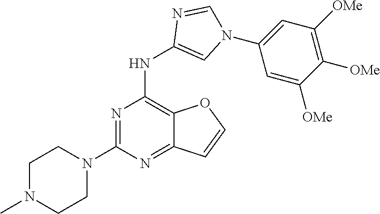 [SMILES: COc1cc(-n2cnc(Nc3nc(N4CCN(C)CC4)nc4ccoc34)c2)cc(OC)c1OC]

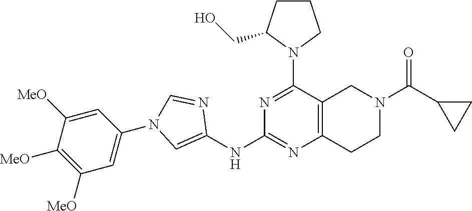 [SMILES: COc1cc(-n2cnc(Nc3nc4c(c(N5CCC[C@H]5CO)n3)CN(C(=O)C3CC3)CC4)c2)cc(OC)c1OC]

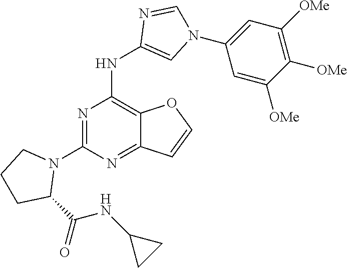 [SMILES: COc1cc(-n2cnc(Nc3nc(N4CCC[C@H]4C(=O)NC4CC4)nc4ccoc34)c2)cc(OC)c1OC]

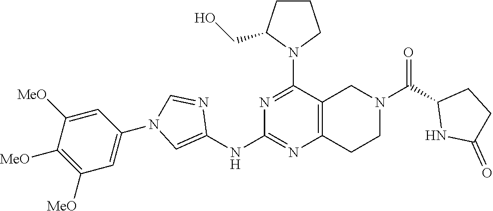 [SMILES: COc1cc(-n2cnc(Nc3nc4c(c(N5CCC[C@H]5CO)n3)CN(C(=O)[C@@H]3CCC(=O)N3)CC4)c2)cc(OC)c1OC]

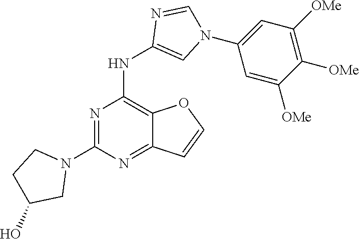 [SMILES: COc1cc(-n2cnc(Nc3nc(N4CC[C@@H](O)C4)nc4ccoc34)c2)cc(OC)c1OC]

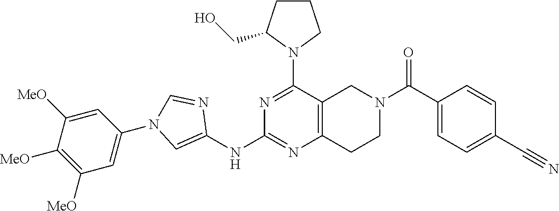 [SMILES: COc1cc(-n2cnc(Nc3nc4c(c(N5CCC[C@H]5CO)n3)CN(C(=O)c3ccc(C#N)cc3)CC4)c2)cc(OC)c1OC]

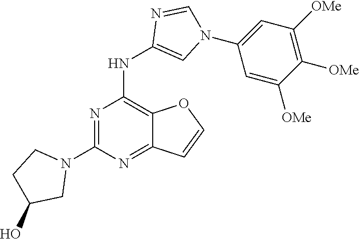 [SMILES: COc1cc(-n2cnc(Nc3nc(N4CC[C@H](O)C4)nc4ccoc34)c2)cc(OC)c1OC]

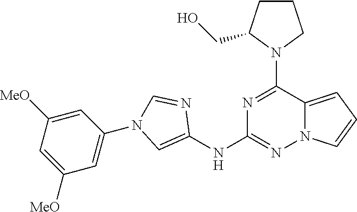 [SMILES: COc1cc(OC)cc(-n2cnc(Nc3nc(N4CCC[C@H]4CO)c4cccn4n3)c2)c1]

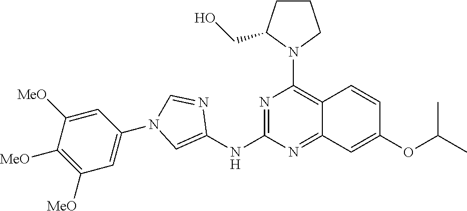 [SMILES: COc1cc(-n2cnc(Nc3nc(N4CCC[C@H]4CO)c4ccc(OC(C)C)cc4n3)c2)cc(OC)c1OC]

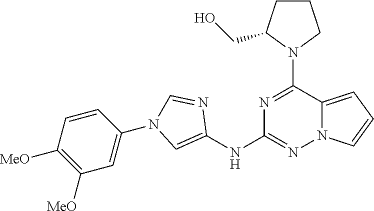 [SMILES: COc1ccc(-n2cnc(Nc3nc(N4CCC[C@H]4CO)c4cccn4n3)c2)cc1OC]

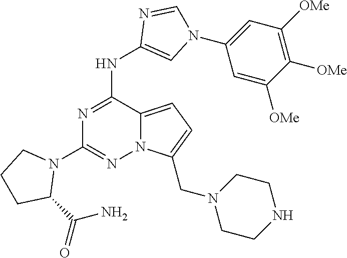 [SMILES: COc1cc(-n2cnc(Nc3nc(N4CCC[C@H]4C(N)=O)nn4c(CN5CCNCC5)ccc34)c2)cc(OC)c1OC]

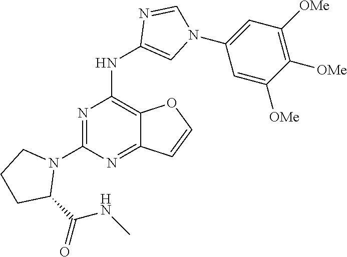 [SMILES: CNC(=O)[C@@H]1CCCN1c1nc(Nc2cn(-c3cc(OC)c(OC)c(OC)c3)cn2)c2occc2n1]

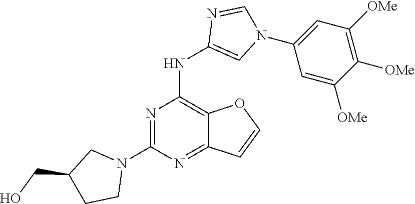 [SMILES: COc1cc(-n2cnc(Nc3nc(N4CC[C@@H](CO)C4)nc4ccoc34)c2)cc(OC)c1OC]

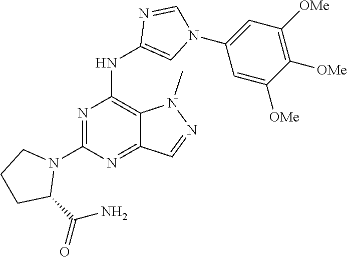 [SMILES: COc1cc(-n2cnc(Nc3nc(N4CCC[C@H]4C(N)=O)nc4cnn(C)c34)c2)cc(OC)c1OC]

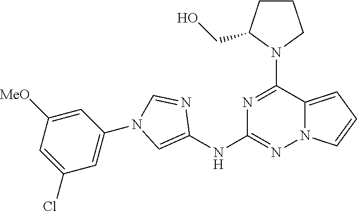 [SMILES: COc1cc(Cl)cc(-n2cnc(Nc3nc(N4CCC[C@H]4CO)c4cccn4n3)c2)c1]

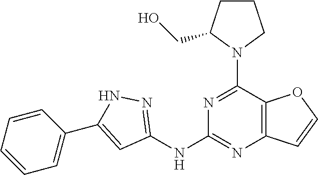 [SMILES: OC[C@@H]1CCCN1c1nc(Nc2cc(-c3ccccc3)[nH]n2)nc2ccoc12]

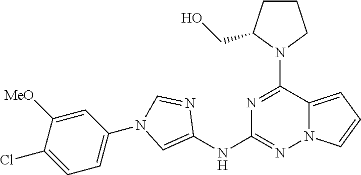 [SMILES: COc1cc(-n2cnc(Nc3nc(N4CCC[C@H]4CO)c4cccn4n3)c2)ccc1Cl]

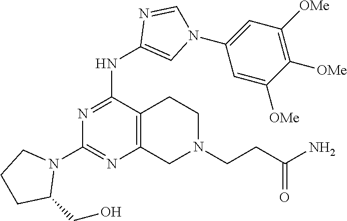 [SMILES: COc1cc(-n2cnc(Nc3nc(N4CCC[C@H]4CO)nc4c3CCN(CCC(N)=O)C4)c2)cc(OC)c1OC]

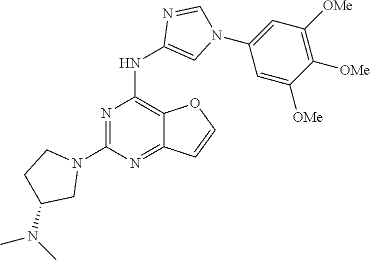 [SMILES: COc1cc(-n2cnc(Nc3nc(N4CC[C@@H](N(C)C)C4)nc4ccoc34)c2)cc(OC)c1OC]

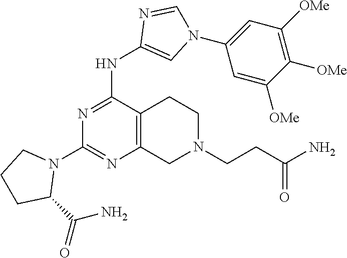 [SMILES: COc1cc(-n2cnc(Nc3nc(N4CCC[C@H]4C(N)=O)nc4c3CCN(CCC(N)=O)C4)c2)cc(OC)c1OC]

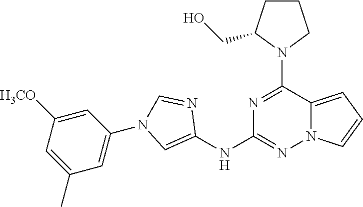 [SMILES: COc1cc(C)cc(-n2cnc(Nc3nc(N4CCC[C@H]4CO)c4cccn4n3)c2)c1]

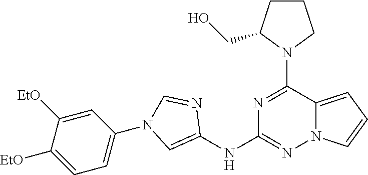 [SMILES: CCOc1ccc(-n2cnc(Nc3nc(N4CCC[C@H]4CO)c4cccn4n3)c2)cc1OCC]

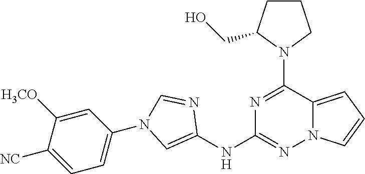 [SMILES: COc1cc(-n2cnc(Nc3nc(N4CCC[C@H]4CO)c4cccn4n3)c2)ccc1C#N]

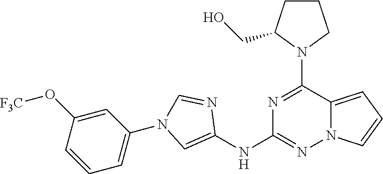 [SMILES: OC[C@@H]1CCCN1c1nc(Nc2cn(-c3cccc(OC(F)(F)F)c3)cn2)nn2cccc12]

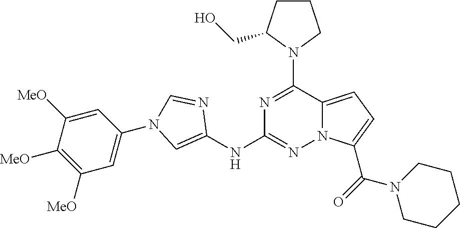 [SMILES: COc1cc(-n2cnc(Nc3nc(N4CCC[C@H]4CO)c4ccc(C(=O)N5CCCCC5)n4n3)c2)cc(OC)c1OC]

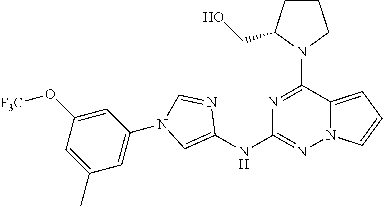 [SMILES: Cc1cc(OC(F)(F)F)cc(-n2cnc(Nc3nc(N4CCC[C@H]4CO)c4cccn4n3)c2)c1]

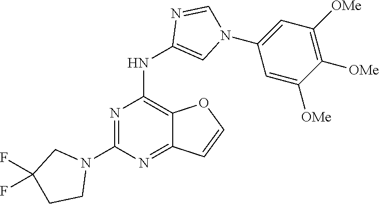 [SMILES: COc1cc(-n2cnc(Nc3nc(N4CCC(F)(F)C4)nc4ccoc34)c2)cc(OC)c1OC]

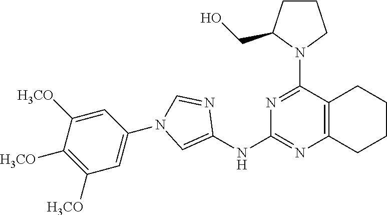 [SMILES: COc1cc(-n2cnc(Nc3nc4c(c(N5CCC[C@@H]5CO)n3)CCCC4)c2)cc(OC)c1OC]

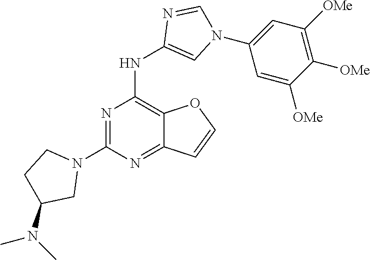 [SMILES: COc1cc(-n2cnc(Nc3nc(N4CC[C@H](N(C)C)C4)nc4ccoc34)c2)cc(OC)c1OC]

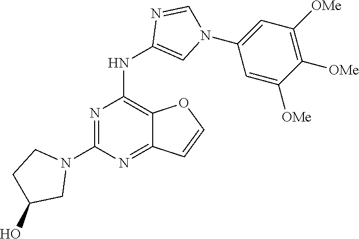 [SMILES: COc1cc(-n2cnc(Nc3nc(N4CC[C@H](O)C4)nc4ccoc34)c2)cc(OC)c1OC]

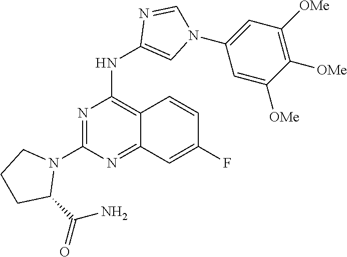 [SMILES: COc1cc(-n2cnc(Nc3nc(N4CCC[C@H]4C(N)=O)nc4cc(F)ccc34)c2)cc(OC)c1OC]

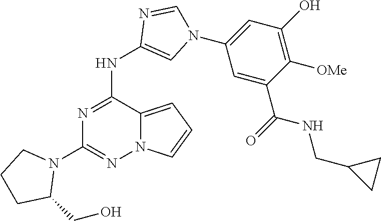 [SMILES: COc1c(O)cc(-n2cnc(Nc3nc(N4CCC[C@H]4CO)nn4cccc34)c2)cc1C(=O)NCC1CC1]

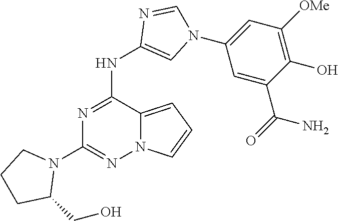 [SMILES: COc1cc(-n2cnc(Nc3nc(N4CCC[C@H]4CO)nn4cccc34)c2)cc(C(N)=O)c1O]

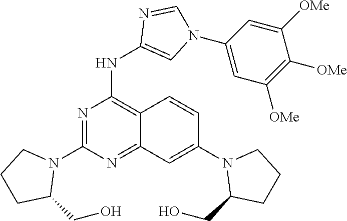 [SMILES: COc1cc(-n2cnc(Nc3nc(N4CCC[C@H]4CO)nc4cc(N5CCC[C@H]5CO)ccc34)c2)cc(OC)c1OC]

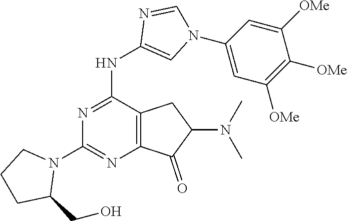 [SMILES: COc1cc(-n2cnc(Nc3nc(N4CCC[C@@H]4CO)nc4c3CC(N(C)C)C4=O)c2)cc(OC)c1OC]

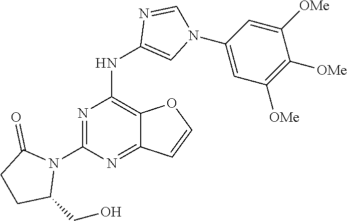 [SMILES: COc1cc(-n2cnc(Nc3nc(N4C(=O)CC[C@H]4CO)nc4ccoc34)c2)cc(OC)c1OC]

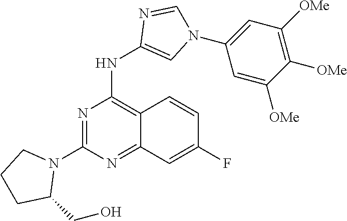 [SMILES: COc1cc(-n2cnc(Nc3nc(N4CCC[C@H]4CO)nc4cc(F)ccc34)c2)cc(OC)c1OC]

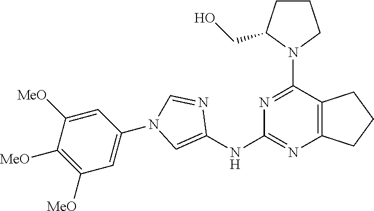 [SMILES: COc1cc(-n2cnc(Nc3nc4c(c(N5CCC[C@H]5CO)n3)CCC4)c2)cc(OC)c1OC]

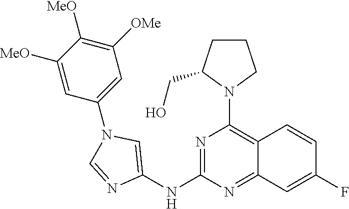 [SMILES: COc1cc(-n2cnc(Nc3nc(N4CCC[C@H]4CO)c4ccc(F)cc4n3)c2)cc(OC)c1OC]

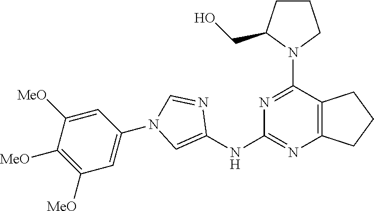 [SMILES: COc1cc(-n2cnc(Nc3nc4c(c(N5CCC[C@@H]5CO)n3)CCC4)c2)cc(OC)c1OC]

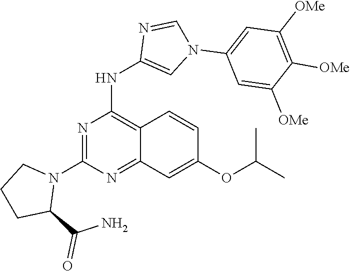 [SMILES: COc1cc(-n2cnc(Nc3nc(N4CCC[C@@H]4C(N)=O)nc4cc(OC(C)C)ccc34)c2)cc(OC)c1OC]

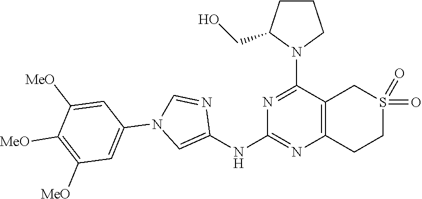 [SMILES: COc1cc(-n2cnc(Nc3nc4c(c(N5CCC[C@H]5CO)n3)CS(=O)(=O)CC4)c2)cc(OC)c1OC]

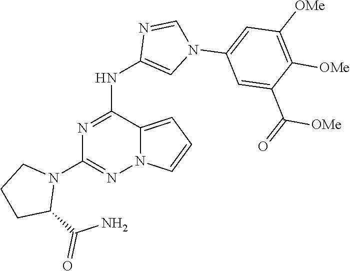 [SMILES: COC(=O)c1cc(-n2cnc(Nc3nc(N4CCC[C@H]4C(N)=O)nn4cccc34)c2)cc(OC)c1OC]

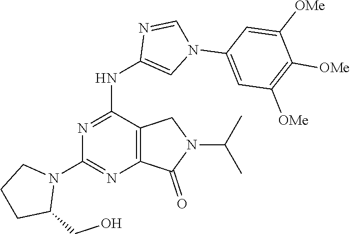 [SMILES: COc1cc(-n2cnc(Nc3nc(N4CCC[C@H]4CO)nc4c3CN(C(C)C)C4=O)c2)cc(OC)c1OC]

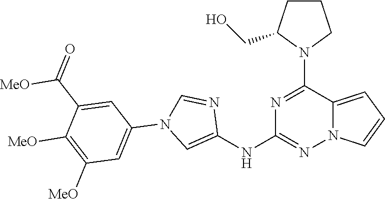 [SMILES: COC(=O)c1cc(-n2cnc(Nc3nc(N4CCC[C@H]4CO)c4cccn4n3)c2)cc(OC)c1OC]

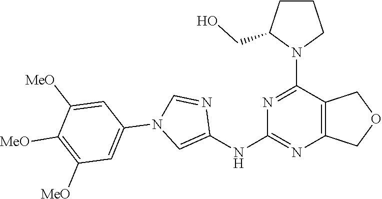 [SMILES: COc1cc(-n2cnc(Nc3nc4c(c(N5CCC[C@H]5CO)n3)COC4)c2)cc(OC)c1OC]

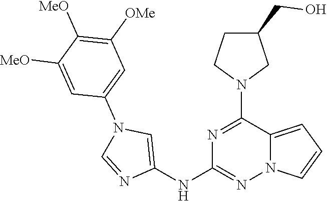 [SMILES: COc1cc(-n2cnc(Nc3nc(N4CC[C@@H](CO)C4)c4cccn4n3)c2)cc(OC)c1OC]

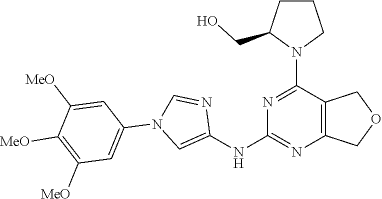 [SMILES: COc1cc(-n2cnc(Nc3nc4c(c(N5CCC[C@@H]5CO)n3)COC4)c2)cc(OC)c1OC]

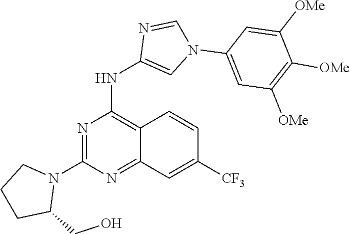 [SMILES: COc1cc(-n2cnc(Nc3nc(N4CCC[C@H]4CO)nc4cc(C(F)(F)F)ccc34)c2)cc(OC)c1OC]

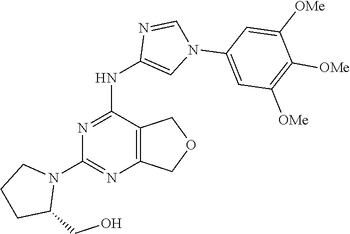 [SMILES: COc1cc(-n2cnc(Nc3nc(N4CCC[C@H]4CO)nc4c3COC4)c2)cc(OC)c1OC]

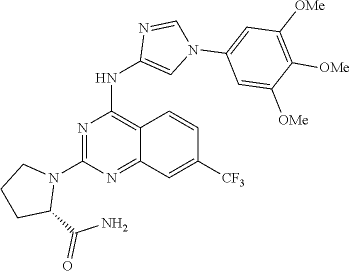 [SMILES: COc1cc(-n2cnc(Nc3nc(N4CCC[C@H]4C(N)=O)nc4cc(C(F)(F)F)ccc34)c2)cc(OC)c1OC]

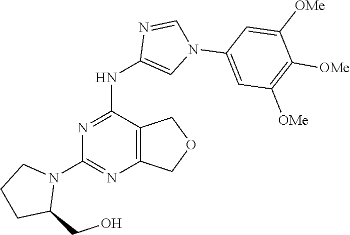 [SMILES: COc1cc(-n2cnc(Nc3nc(N4CCC[C@@H]4CO)nc4c3COC4)c2)cc(OC)c1OC]

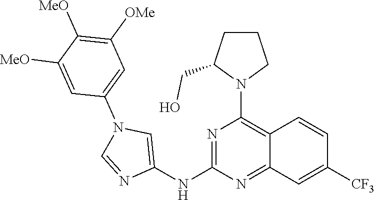 [SMILES: COc1cc(-n2cnc(Nc3nc(N4CCC[C@H]4CO)c4ccc(C(F)(F)F)cc4n3)c2)cc(OC)c1OC]

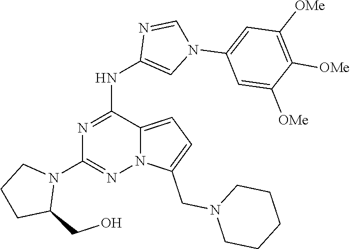 [SMILES: COc1cc(-n2cnc(Nc3nc(N4CCC[C@@H]4CO)nn4c(CN5CCCCC5)ccc34)c2)cc(OC)c1OC]

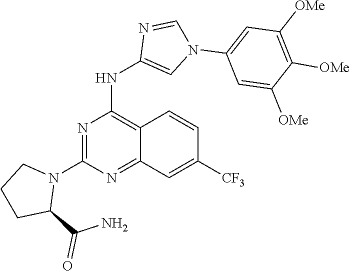 [SMILES: COc1cc(-n2cnc(Nc3nc(N4CCC[C@@H]4C(N)=O)nc4cc(C(F)(F)F)ccc34)c2)cc(OC)c1OC]

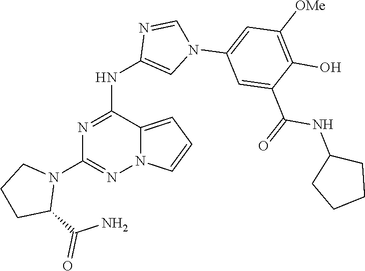 [SMILES: COc1cc(-n2cnc(Nc3nc(N4CCC[C@H]4C(N)=O)nn4cccc34)c2)cc(C(=O)NC2CCCC2)c1O]

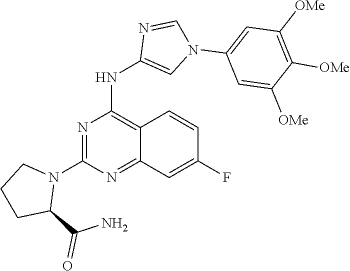 [SMILES: COc1cc(-n2cnc(Nc3nc(N4CCC[C@@H]4C(N)=O)nc4cc(F)ccc34)c2)cc(OC)c1OC]

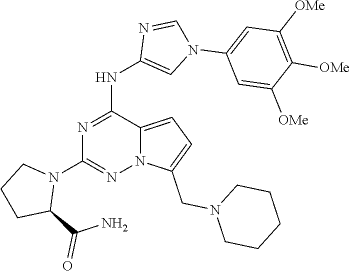 [SMILES: COc1cc(-n2cnc(Nc3nc(N4CCC[C@@H]4C(N)=O)nn4c(CN5CCCCC5)ccc34)c2)cc(OC)c1OC]

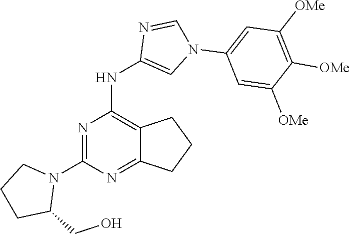 [SMILES: COc1cc(-n2cnc(Nc3nc(N4CCC[C@H]4CO)nc4c3CCC4)c2)cc(OC)c1OC]

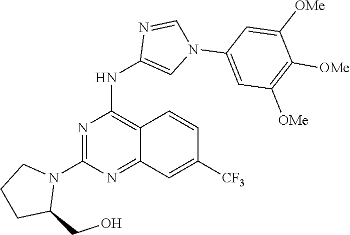 [SMILES: COc1cc(-n2cnc(Nc3nc(N4CCC[C@@H]4CO)nc4cc(C(F)(F)F)ccc34)c2)cc(OC)c1OC]

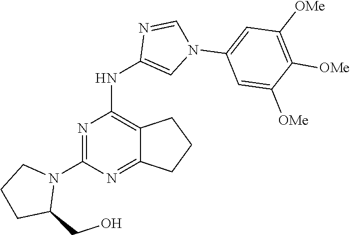 [SMILES: COc1cc(-n2cnc(Nc3nc(N4CCC[C@@H]4CO)nc4c3CCC4)c2)cc(OC)c1OC]

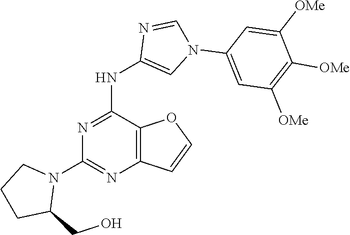 [SMILES: COc1cc(-n2cnc(Nc3nc(N4CCC[C@@H]4CO)nc4ccoc34)c2)cc(OC)c1OC]

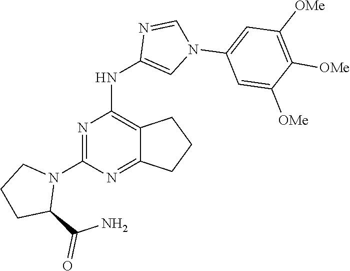 [SMILES: COc1cc(-n2cnc(Nc3nc(N4CCC[C@@H]4C(N)=O)nc4c3CCC4)c2)cc(OC)c1OC]

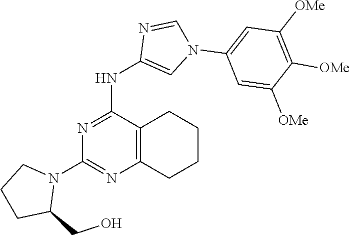 [SMILES: COc1cc(-n2cnc(Nc3nc(N4CCC[C@@H]4CO)nc4c3CCCC4)c2)cc(OC)c1OC]

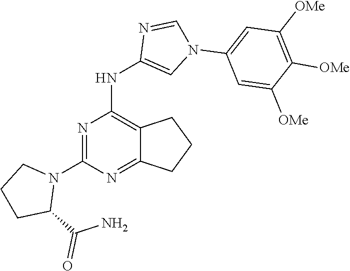 [SMILES: COc1cc(-n2cnc(Nc3nc(N4CCC[C@H]4C(N)=O)nc4c3CCC4)c2)cc(OC)c1OC]

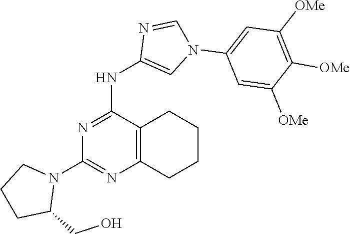 [SMILES: COc1cc(-n2cnc(Nc3nc(N4CCC[C@H]4CO)nc4c3CCCC4)c2)cc(OC)c1OC]

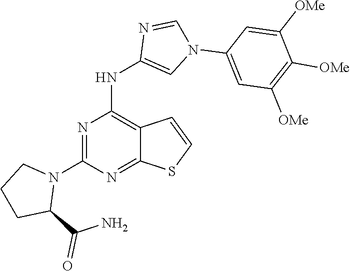 [SMILES: COc1cc(-n2cnc(Nc3nc(N4CCC[C@@H]4C(N)=O)nc4sccc34)c2)cc(OC)c1OC]

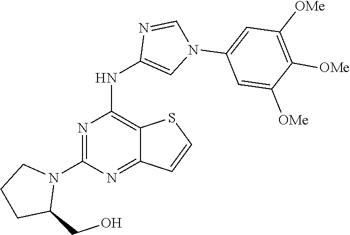 [SMILES: COc1cc(-n2cnc(Nc3nc(N4CCC[C@@H]4CO)nc4ccsc34)c2)cc(OC)c1OC]

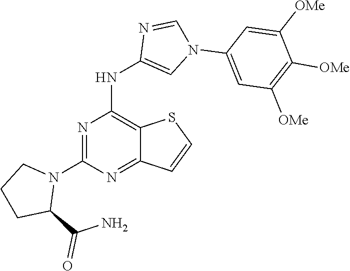 [SMILES: COc1cc(-n2cnc(Nc3nc(N4CCC[C@@H]4C(N)=O)nc4ccsc34)c2)cc(OC)c1OC]

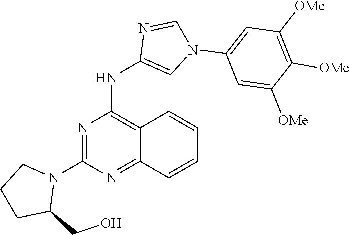 [SMILES: COc1cc(-n2cnc(Nc3nc(N4CCC[C@@H]4CO)nc4ccccc34)c2)cc(OC)c1OC]

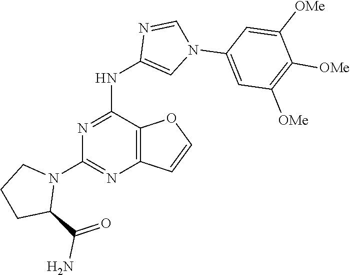 [SMILES: COc1cc(-n2cnc(Nc3nc(N4CCC[C@@H]4C(N)=O)nc4ccoc34)c2)cc(OC)c1OC]

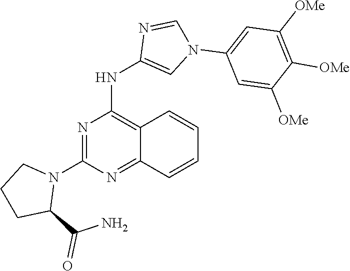 [SMILES: COc1cc(-n2cnc(Nc3nc(N4CCC[C@@H]4C(N)=O)nc4ccccc34)c2)cc(OC)c1OC]

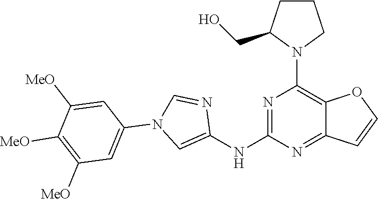 [SMILES: COc1cc(-n2cnc(Nc3nc(N4CCC[C@@H]4CO)c4occc4n3)c2)cc(OC)c1OC]

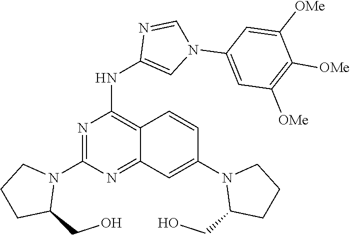 [SMILES: COc1cc(-n2cnc(Nc3nc(N4CCC[C@@H]4CO)nc4cc(N5CCC[C@@H]5CO)ccc34)c2)cc(OC)c1OC]

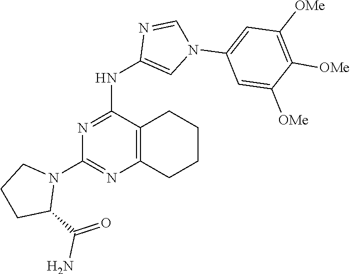 [SMILES: COc1cc(-n2cnc(Nc3nc(N4CCC[C@H]4C(N)=O)nc4c3CCCC4)c2)cc(OC)c1OC]

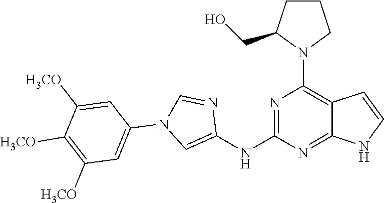 [SMILES: COc1cc(-n2cnc(Nc3nc(N4CCC[C@@H]4CO)c4cc[nH]c4n3)c2)cc(OC)c1OC]

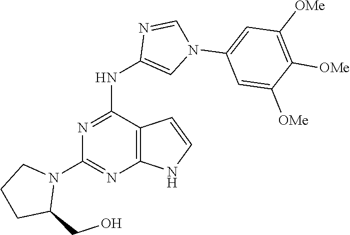 [SMILES: COc1cc(-n2cnc(Nc3nc(N4CCC[C@@H]4CO)nc4[nH]ccc34)c2)cc(OC)c1OC]

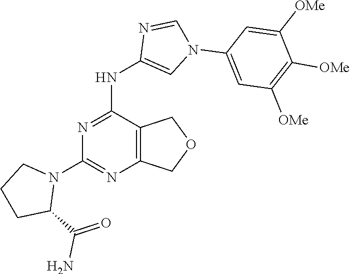 [SMILES: COc1cc(-n2cnc(Nc3nc(N4CCC[C@H]4C(N)=O)nc4c3COC4)c2)cc(OC)c1OC]

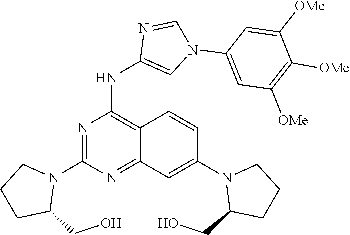 [SMILES: COc1cc(-n2cnc(Nc3nc(N4CCC[C@H]4CO)nc4cc(N5CCC[C@H]5CO)ccc34)c2)cc(OC)c1OC]

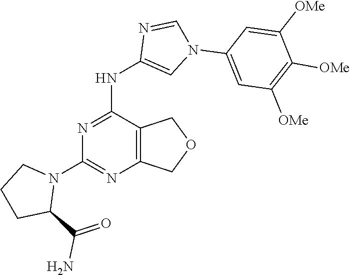 [SMILES: COc1cc(-n2cnc(Nc3nc(N4CCC[C@@H]4C(N)=O)nc4c3COC4)c2)cc(OC)c1OC]

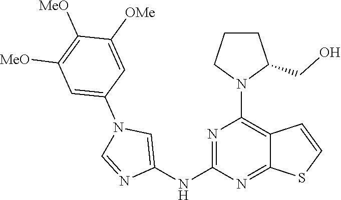 [SMILES: COc1cc(-n2cnc(Nc3nc(N4CCC[C@@H]4CO)c4ccsc4n3)c2)cc(OC)c1OC]

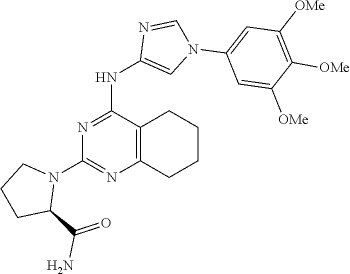 [SMILES: COc1cc(-n2cnc(Nc3nc(N4CCC[C@@H]4C(N)=O)nc4c3CCCC4)c2)cc(OC)c1OC]

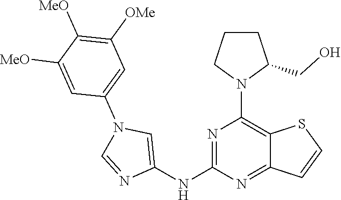 [SMILES: COc1cc(-n2cnc(Nc3nc(N4CCC[C@@H]4CO)c4sccc4n3)c2)cc(OC)c1OC]

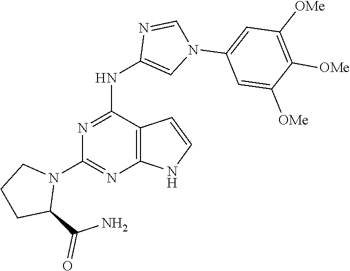 [SMILES: COc1cc(-n2cnc(Nc3nc(N4CCC[C@@H]4C(N)=O)nc4[nH]ccc34)c2)cc(OC)c1OC]

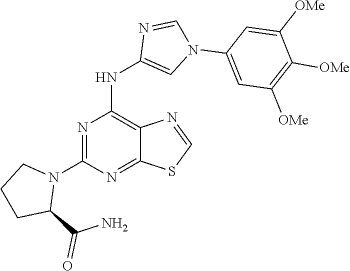 [SMILES: COc1cc(-n2cnc(Nc3nc(N4CCC[C@@H]4C(N)=O)nc4scnc34)c2)cc(OC)c1OC]

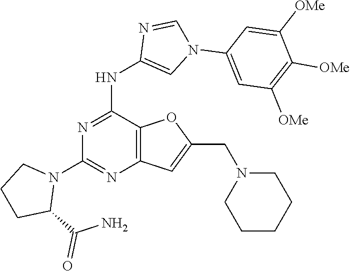 [SMILES: COc1cc(-n2cnc(Nc3nc(N4CCC[C@H]4C(N)=O)nc4cc(CN5CCCCC5)oc34)c2)cc(OC)c1OC]

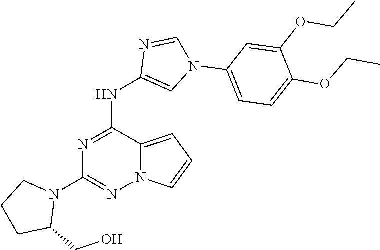 [SMILES: CCOc1ccc(-n2cnc(Nc3nc(N4CCC[C@H]4CO)nn4cccc34)c2)cc1OCC]

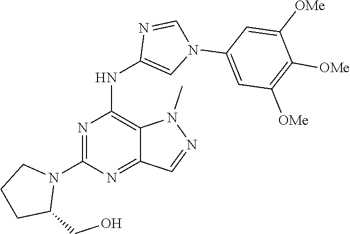 [SMILES: COc1cc(-n2cnc(Nc3nc(N4CCC[C@H]4CO)nc4cnn(C)c34)c2)cc(OC)c1OC]

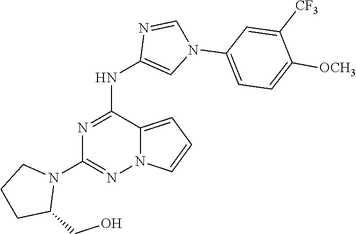 [SMILES: COc1ccc(-n2cnc(Nc3nc(N4CCC[C@H]4CO)nn4cccc34)c2)cc1C(F)(F)F]

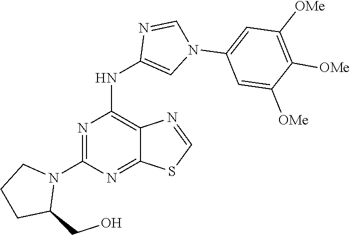 [SMILES: COc1cc(-n2cnc(Nc3nc(N4CCC[C@@H]4CO)nc4scnc34)c2)cc(OC)c1OC]

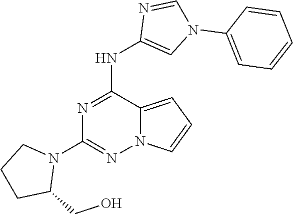 [SMILES: OC[C@@H]1CCCN1c1nc(Nc2cn(-c3ccccc3)cn2)c2cccn2n1]

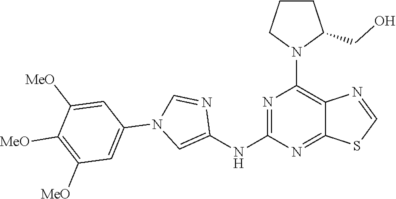 [SMILES: COc1cc(-n2cnc(Nc3nc(N4CCC[C@@H]4CO)c4ncsc4n3)c2)cc(OC)c1OC]

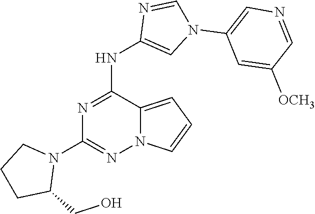 [SMILES: COc1cncc(-n2cnc(Nc3nc(N4CCC[C@H]4CO)nn4cccc34)c2)c1]

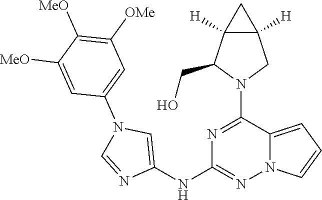 [SMILES: COc1cc(-n2cnc(Nc3nc(N4C[C@@H]5C[C@@H]5[C@@H]4CO)c4cccn4n3)c2)cc(OC)c1OC]

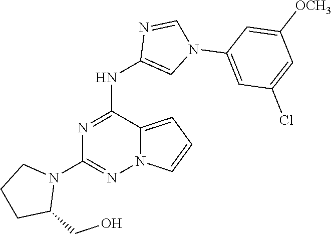 [SMILES: COc1cc(Cl)cc(-n2cnc(Nc3nc(N4CCC[C@H]4CO)nn4cccc34)c2)c1]

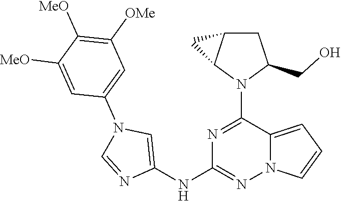 [SMILES: COc1cc(-n2cnc(Nc3nc(N4[C@H](CO)C[C@H]5C[C@H]54)c4cccn4n3)c2)cc(OC)c1OC]

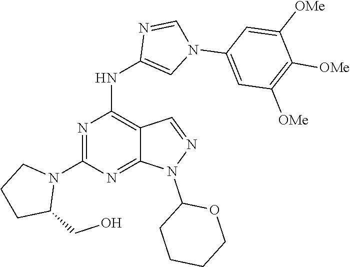 [SMILES: COc1cc(-n2cnc(Nc3nc(N4CCC[C@H]4CO)nc4c3cnn4C3CCCCO3)c2)cc(OC)c1OC]

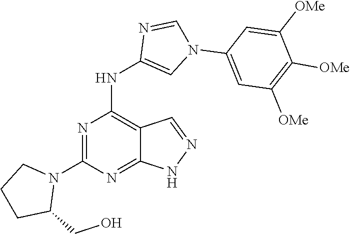 [SMILES: COc1cc(-n2cnc(Nc3nc(N4CCC[C@H]4CO)nc4[nH]ncc34)c2)cc(OC)c1OC]

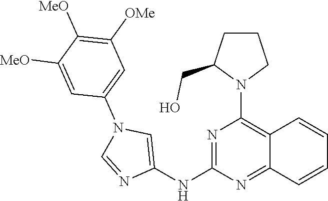 [SMILES: COc1cc(-n2cnc(Nc3nc(N4CCC[C@@H]4CO)c4ccccc4n3)c2)cc(OC)c1OC]

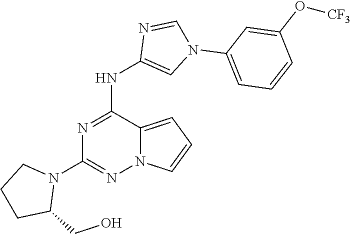 [SMILES: OC[C@@H]1CCCN1c1nc(Nc2cn(-c3cccc(OC(F)(F)F)c3)cn2)c2cccn2n1]

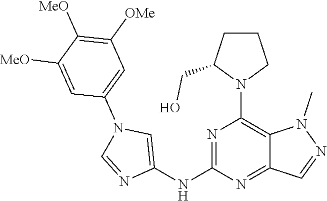 [SMILES: COc1cc(-n2cnc(Nc3nc(N4CCC[C@H]4CO)c4c(cnn4C)n3)c2)cc(OC)c1OC]

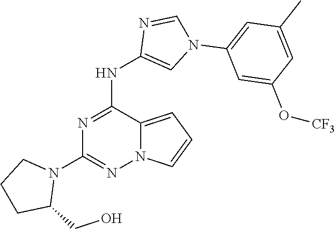 [SMILES: Cc1cc(OC(F)(F)F)cc(-n2cnc(Nc3nc(N4CCC[C@H]4CO)nn4cccc34)c2)c1]

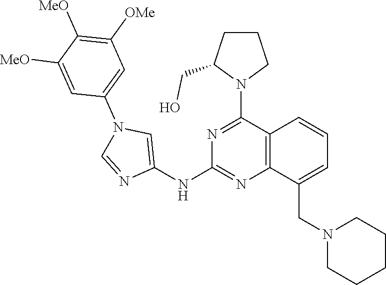 [SMILES: COc1cc(-n2cnc(Nc3nc(N4CCC[C@H]4CO)c4cccc(CN5CCCCC5)c4n3)c2)cc(OC)c1OC]

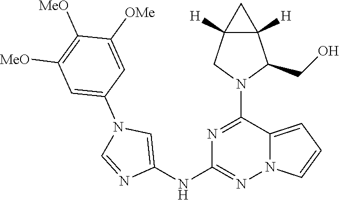 [SMILES: COc1cc(-n2cnc(Nc3nc(N4C[C@@H]5C[C@@H]5[C@H]4CO)c4cccn4n3)c2)cc(OC)c1OC]

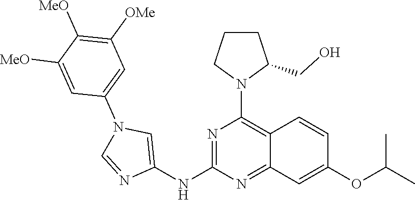 [SMILES: COc1cc(-n2cnc(Nc3nc(N4CCC[C@@H]4CO)c4ccc(OC(C)C)cc4n3)c2)cc(OC)c1OC]

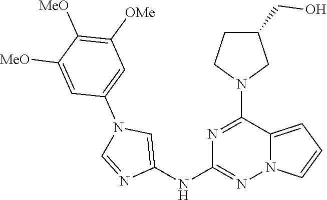 [SMILES: COc1cc(-n2cnc(Nc3nc(N4CC[C@H](CO)C4)c4cccn4n3)c2)cc(OC)c1OC]

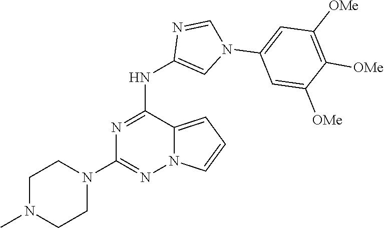 [SMILES: COc1cc(-n2cnc(Nc3nc(N4CCN(C)CC4)nn4cccc34)c2)cc(OC)c1OC]

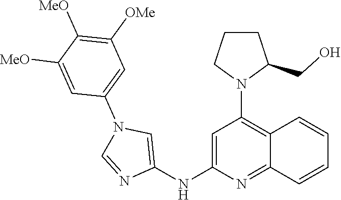 [SMILES: COc1cc(-n2cnc(Nc3cc(N4CCC[C@H]4CO)c4ccccc4n3)c2)cc(OC)c1OC]

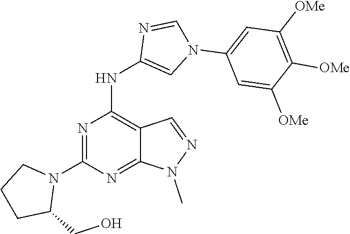 [SMILES: COc1cc(-n2cnc(Nc3nc(N4CCC[C@H]4CO)nc4c3cnn4C)c2)cc(OC)c1OC]

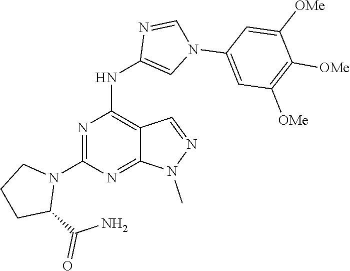 [SMILES: COc1cc(-n2cnc(Nc3nc(N4CCC[C@H]4C(N)=O)nc4c3cnn4C)c2)cc(OC)c1OC]

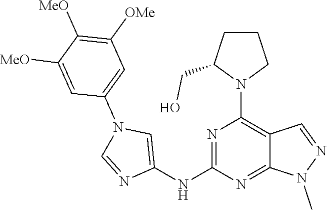 [SMILES: COc1cc(-n2cnc(Nc3nc(N4CCC[C@H]4CO)c4cnn(C)c4n3)c2)cc(OC)c1OC]

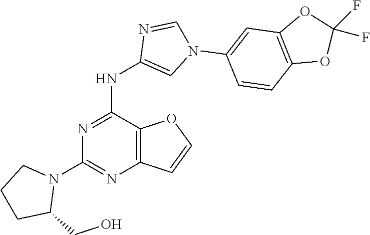 [SMILES: OC[C@@H]1CCCN1c1nc(Nc2cn(-c3ccc4c(c3)OC(F)(F)O4)cn2)c2occc2n1]

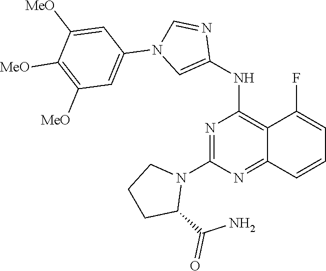 [SMILES: COc1cc(-n2cnc(Nc3nc(N4CCC[C@H]4C(N)=O)nc4cccc(F)c34)c2)cc(OC)c1OC]

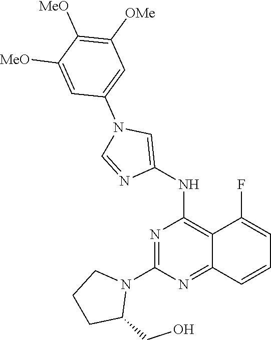 [SMILES: COc1cc(-n2cnc(Nc3nc(N4CCC[C@H]4CO)nc4cccc(F)c34)c2)cc(OC)c1OC]

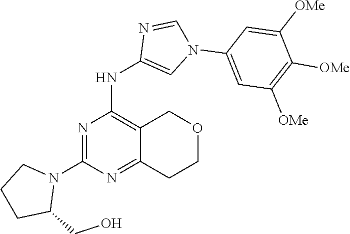 [SMILES: COc1cc(-n2cnc(Nc3nc(N4CCC[C@H]4CO)nc4c3COCC4)c2)cc(OC)c1OC]

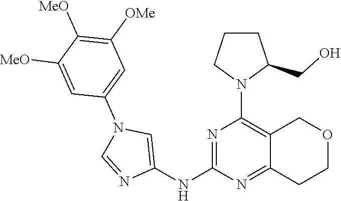 [SMILES: COc1cc(-n2cnc(Nc3nc4c(c(N5CCC[C@H]5CO)n3)COCC4)c2)cc(OC)c1OC]

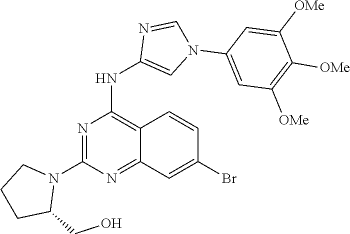 [SMILES: COc1cc(-n2cnc(Nc3nc(N4CCC[C@H]4CO)nc4cc(Br)ccc34)c2)cc(OC)c1OC]

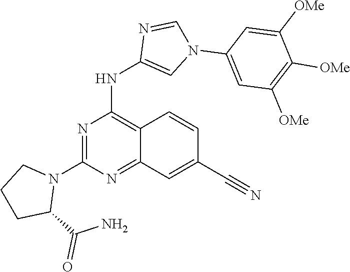 [SMILES: COc1cc(-n2cnc(Nc3nc(N4CCC[C@H]4C(N)=O)nc4cc(C#N)ccc34)c2)cc(OC)c1OC]

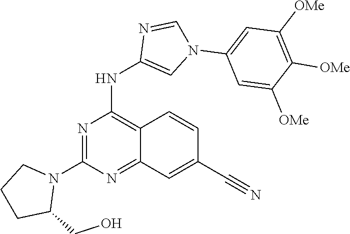 [SMILES: COc1cc(-n2cnc(Nc3nc(N4CCC[C@H]4CO)nc4cc(C#N)ccc34)c2)cc(OC)c1OC]

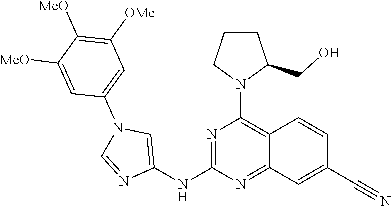 [SMILES: COc1cc(-n2cnc(Nc3nc(N4CCC[C@H]4CO)c4ccc(C#N)cc4n3)c2)cc(OC)c1OC]

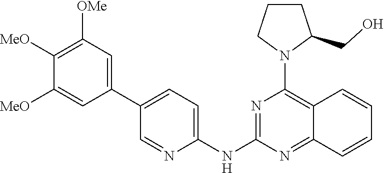 [SMILES: COc1cc(-c2ccc(Nc3nc(N4CCC[C@H]4CO)c4ccccc4n3)nc2)cc(OC)c1OC]

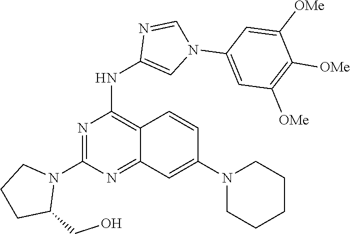 [SMILES: COc1cc(-n2cnc(Nc3nc(N4CCC[C@H]4CO)nc4cc(N5CCCCC5)ccc34)c2)cc(OC)c1OC]

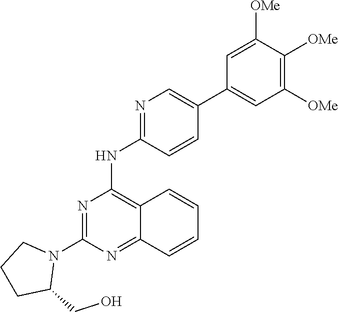 [SMILES: COc1cc(-c2ccc(Nc3nc(N4CCC[C@H]4CO)nc4ccccc34)nc2)cc(OC)c1OC]

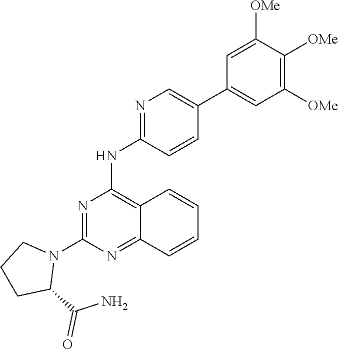 [SMILES: COc1cc(-c2ccc(Nc3nc(N4CCC[C@H]4C(N)=O)nc4ccccc34)nc2)cc(OC)c1OC]

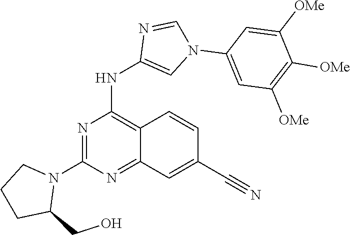 [SMILES: COc1cc(-n2cnc(Nc3nc(N4CCC[C@@H]4CO)nc4cc(C#N)ccc34)c2)cc(OC)c1OC]

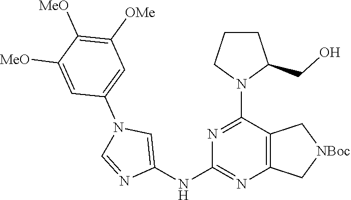 [SMILES: COc1cc(-n2cnc(Nc3nc4c(c(N5CCC[C@H]5CO)n3)CN(C(=O)OC(C)(C)C)C4)c2)cc(OC)c1OC]

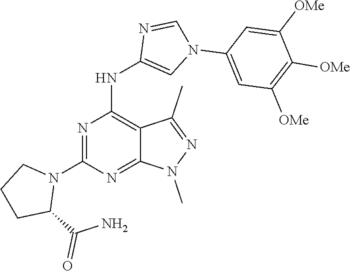 [SMILES: COc1cc(-n2cnc(Nc3nc(N4CCC[C@H]4C(N)=O)nc4c3c(C)nn4C)c2)cc(OC)c1OC]

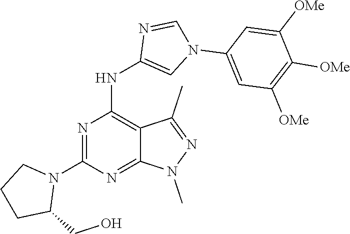 [SMILES: COc1cc(-n2cnc(Nc3nc(N4CCC[C@H]4CO)nc4c3c(C)nn4C)c2)cc(OC)c1OC]

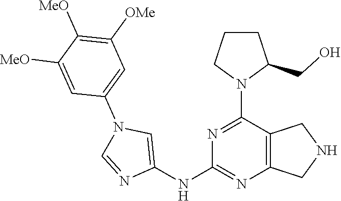 [SMILES: COc1cc(-n2cnc(Nc3nc4c(c(N5CCC[C@H]5CO)n3)CNC4)c2)cc(OC)c1OC]

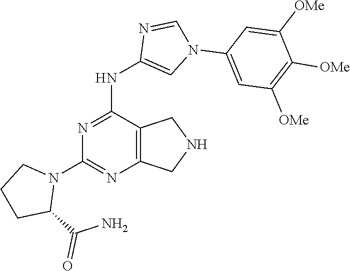 [SMILES: COc1cc(-n2cnc(Nc3nc(N4CCC[C@H]4C(N)=O)nc4c3CNC4)c2)cc(OC)c1OC]

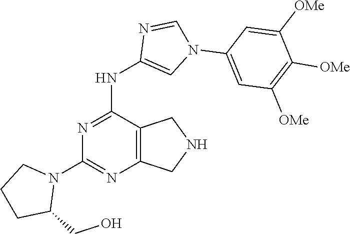 [SMILES: COc1cc(-n2cnc(Nc3nc(N4CCC[C@H]4CO)nc4c3CNC4)c2)cc(OC)c1OC]

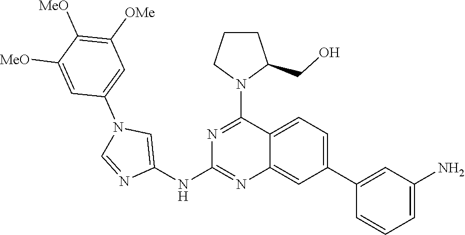 [SMILES: COc1cc(-n2cnc(Nc3nc(N4CCC[C@H]4CO)c4ccc(-c5cccc(N)c5)cc4n3)c2)cc(OC)c1OC]

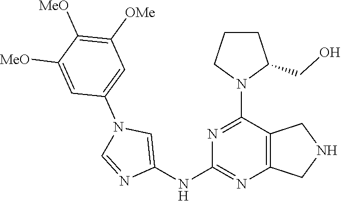 [SMILES: COc1cc(-n2cnc(Nc3nc4c(c(N5CCC[C@@H]5CO)n3)CNC4)c2)cc(OC)c1OC]

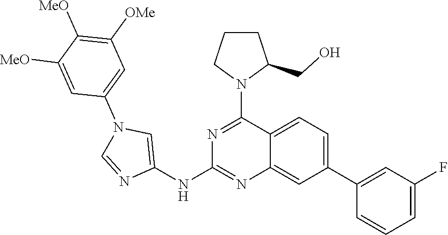 [SMILES: COc1cc(-n2cnc(Nc3nc(N4CCC[C@H]4CO)c4ccc(-c5cccc(F)c5)cc4n3)c2)cc(OC)c1OC]

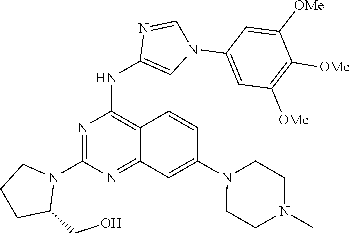 [SMILES: COc1cc(-n2cnc(Nc3nc(N4CCC[C@H]4CO)nc4cc(N5CCN(C)CC5)ccc34)c2)cc(OC)c1OC]

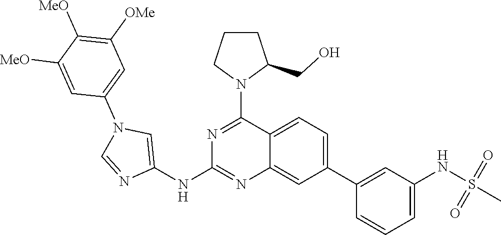 [SMILES: COc1cc(-n2cnc(Nc3nc(N4CCC[C@H]4CO)c4ccc(-c5cccc(NS(C)(=O)=O)c5)cc4n3)c2)cc(OC)c1OC]

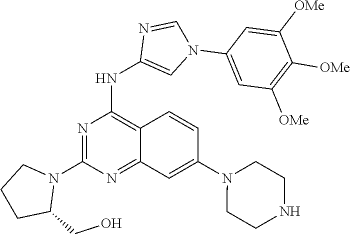 [SMILES: COc1cc(-n2cnc(Nc3nc(N4CCC[C@H]4CO)nc4cc(N5CCNCC5)ccc34)c2)cc(OC)c1OC]

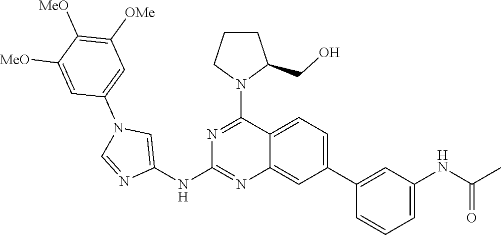 [SMILES: COc1cc(-n2cnc(Nc3nc(N4CCC[C@H]4CO)c4ccc(-c5cccc(NC(C)=O)c5)cc4n3)c2)cc(OC)c1OC]

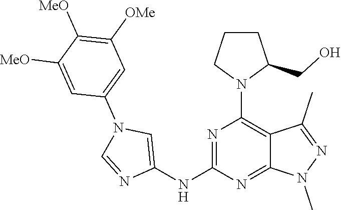 [SMILES: COc1cc(-n2cnc(Nc3nc(N4CCC[C@H]4CO)c4c(C)nn(C)c4n3)c2)cc(OC)c1OC]

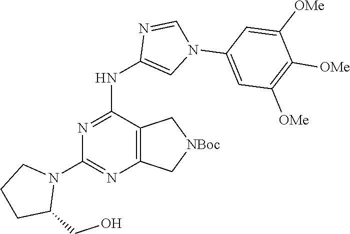 [SMILES: COc1cc(-n2cnc(Nc3nc(N4CCC[C@H]4CO)nc4c3CN(C(=O)OC(C)(C)C)C4)c2)cc(OC)c1OC]

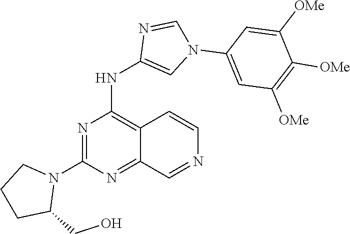 [SMILES: COc1cc(-n2cnc(Nc3nc(N4CCC[C@H]4CO)nc4cnccc34)c2)cc(OC)c1OC]

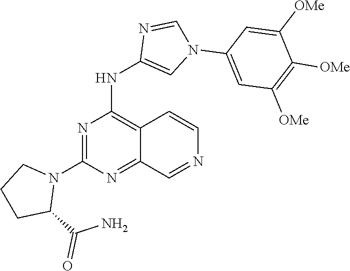 [SMILES: COc1cc(-n2cnc(Nc3nc(N4CCC[C@H]4C(N)=O)nc4cnccc34)c2)cc(OC)c1OC]

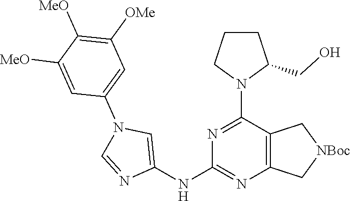 [SMILES: COc1cc(-n2cnc(Nc3nc4c(c(N5CCC[C@@H]5CO)n3)CN(C(=O)OC(C)(C)C)C4)c2)cc(OC)c1OC]

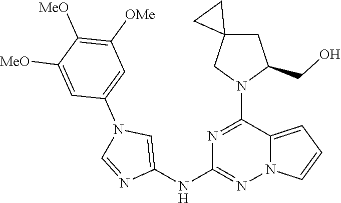 [SMILES: COc1cc(-n2cnc(Nc3nc(N4CC5(CC5)C[C@H]4CO)c4cccn4n3)c2)cc(OC)c1OC]

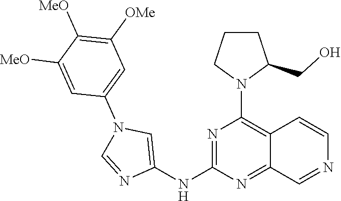 [SMILES: COc1cc(-n2cnc(Nc3nc(N4CCC[C@H]4CO)c4ccncc4n3)c2)cc(OC)c1OC]

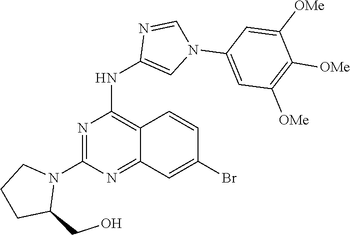 [SMILES: COc1cc(-n2cnc(Nc3nc(N4CCC[C@@H]4CO)nc4cc(Br)ccc34)c2)cc(OC)c1OC]

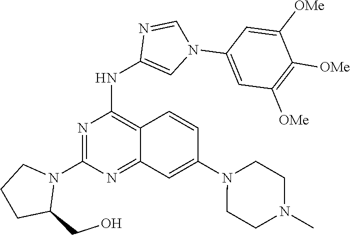 [SMILES: COc1cc(-n2cnc(Nc3nc(N4CCC[C@@H]4CO)nc4cc(N5CCN(C)CC5)ccc34)c2)cc(OC)c1OC]

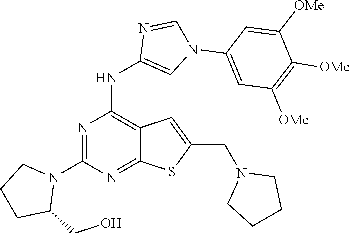 [SMILES: COc1cc(-n2cnc(Nc3nc(N4CCC[C@H]4CO)nc4sc(CN5CCCC5)cc34)c2)cc(OC)c1OC]

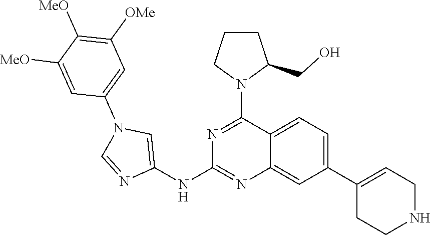 [SMILES: COc1cc(-n2cnc(Nc3nc(N4CCC[C@H]4CO)c4ccc(C5=CCNCC5)cc4n3)c2)cc(OC)c1OC]

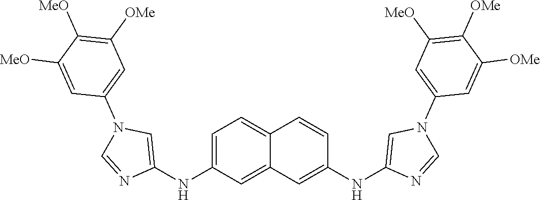 [SMILES: COc1cc(-n2cnc(Nc3ccc4ccc(Nc5cn(-c6cc(OC)c(OC)c(OC)c6)cn5)cc4c3)c2)cc(OC)c1OC]

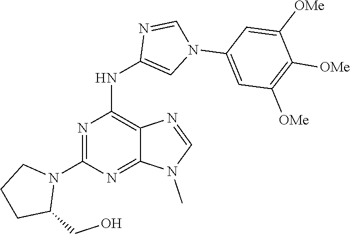 [SMILES: COc1cc(-n2cnc(Nc3nc(N4CCC[C@H]4CO)nc4c3ncn4C)c2)cc(OC)c1OC]

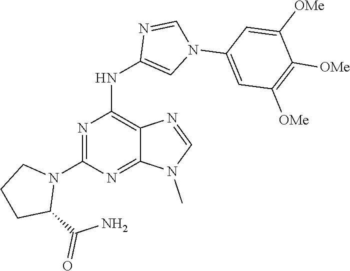 [SMILES: COc1cc(-n2cnc(Nc3nc(N4CCC[C@H]4C(N)=O)nc4c3ncn4C)c2)cc(OC)c1OC]

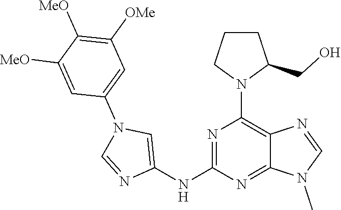 [SMILES: COc1cc(-n2cnc(Nc3nc(N4CCC[C@H]4CO)c4ncn(C)c4n3)c2)cc(OC)c1OC]

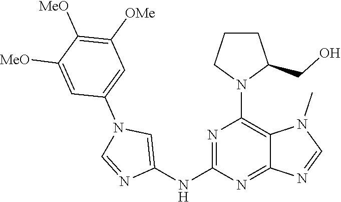 [SMILES: COc1cc(-n2cnc(Nc3nc(N4CCC[C@H]4CO)c4c(ncn4C)n3)c2)cc(OC)c1OC]

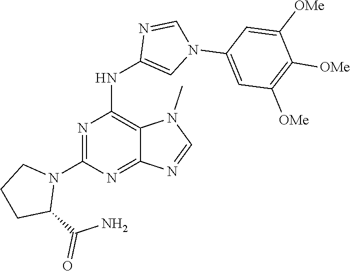 [SMILES: COc1cc(-n2cnc(Nc3nc(N4CCC[C@H]4C(N)=O)nc4ncn(C)c34)c2)cc(OC)c1OC]

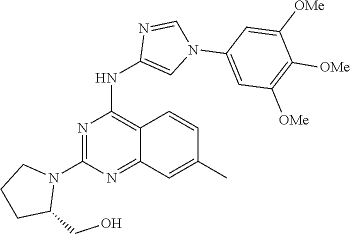 [SMILES: COc1cc(-n2cnc(Nc3nc(N4CCC[C@H]4CO)nc4cc(C)ccc34)c2)cc(OC)c1OC]

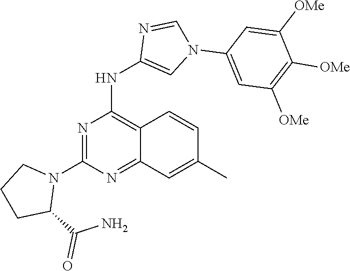 [SMILES: COc1cc(-n2cnc(Nc3nc(N4CCC[C@H]4C(N)=O)nc4cc(C)ccc34)c2)cc(OC)c1OC]

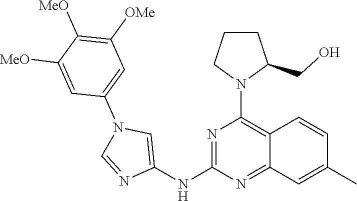 [SMILES: COc1cc(-n2cnc(Nc3nc(N4CCC[C@H]4CO)c4ccc(C)cc4n3)c2)cc(OC)c1OC]

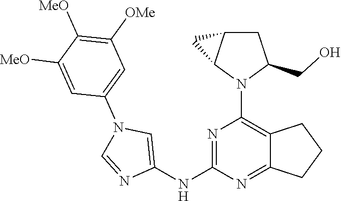 [SMILES: COc1cc(-n2cnc(Nc3nc4c(c(N5[C@H](CO)C[C@H]6C[C@H]65)n3)CCC4)c2)cc(OC)c1OC]

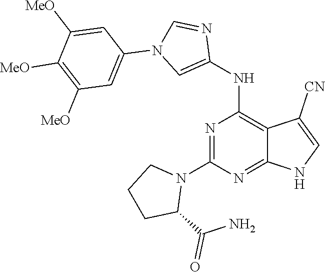 [SMILES: COc1cc(-n2cnc(Nc3nc(N4CCC[C@H]4C(N)=O)nc4[nH]cc(C#N)c34)c2)cc(OC)c1OC]

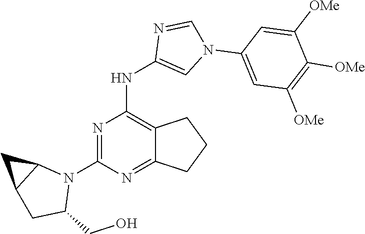 [SMILES: COc1cc(-n2cnc(Nc3nc(N4[C@H](CO)C[C@H]5C[C@H]54)nc4c3CCC4)c2)cc(OC)c1OC]

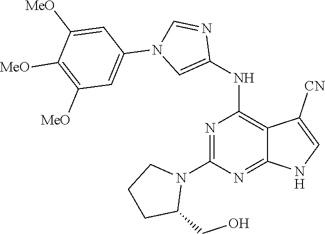 [SMILES: COc1cc(-n2cnc(Nc3nc(N4CCC[C@H]4CO)nc4[nH]cc(C#N)c34)c2)cc(OC)c1OC]

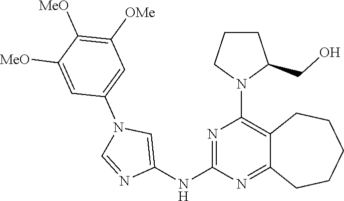 [SMILES: COc1cc(-n2cnc(Nc3nc4c(c(N5CCC[C@H]5CO)n3)CCCCC4)c2)cc(OC)c1OC]

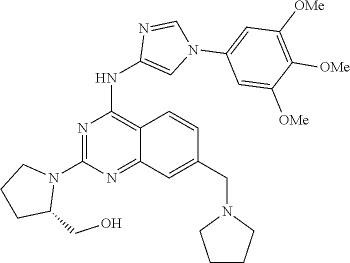 [SMILES: COc1cc(-n2cnc(Nc3nc(N4CCC[C@H]4CO)nc4cc(CN5CCCC5)ccc34)c2)cc(OC)c1OC]

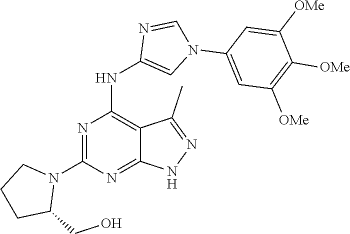 [SMILES: COc1cc(-n2cnc(Nc3nc(N4CCC[C@H]4CO)nc4[nH]nc(C)c34)c2)cc(OC)c1OC]

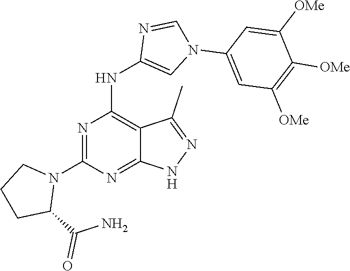 [SMILES: COc1cc(-n2cnc(Nc3nc(N4CCC[C@H]4C(N)=O)nc4[nH]nc(C)c34)c2)cc(OC)c1OC]

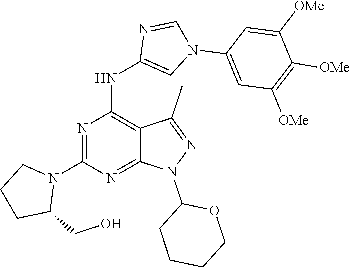 [SMILES: COc1cc(-n2cnc(Nc3nc(N4CCC[C@H]4CO)nc4c3c(C)nn4C3CCCCO3)c2)cc(OC)c1OC]

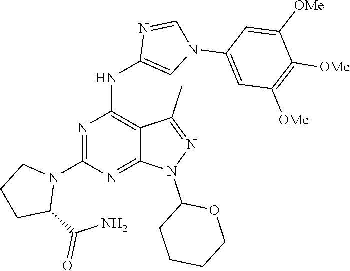 [SMILES: COc1cc(-n2cnc(Nc3nc(N4CCC[C@H]4C(N)=O)nc4c3c(C)nn4C3CCCCO3)c2)cc(OC)c1OC]

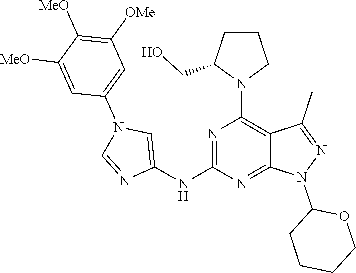 [SMILES: COc1cc(-n2cnc(Nc3nc(N4CCC[C@H]4CO)c4c(C)nn(C5CCCCO5)c4n3)c2)cc(OC)c1OC]

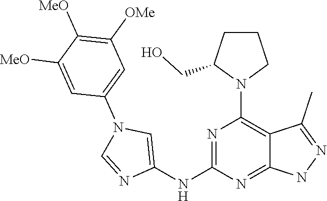 [SMILES: COc1cc(-n2cnc(Nc3nc(N4CCC[C@H]4CO)c4c(C)n[nH]c4n3)c2)cc(OC)c1OC]

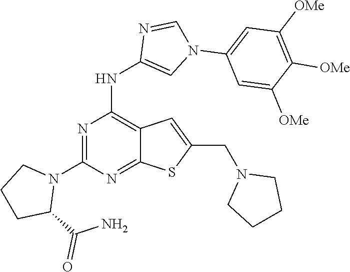 [SMILES: COc1cc(-n2cnc(Nc3nc(N4CCC[C@H]4C(N)=O)nc4sc(CN5CCCC5)cc34)c2)cc(OC)c1OC]

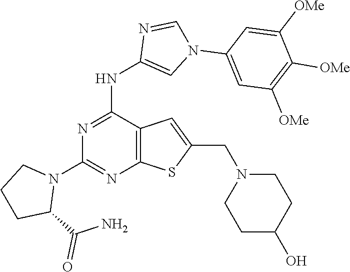 [SMILES: COc1cc(-n2cnc(Nc3nc(N4CCC[C@H]4C(N)=O)nc4sc(CN5CCC(O)CC5)cc34)c2)cc(OC)c1OC]

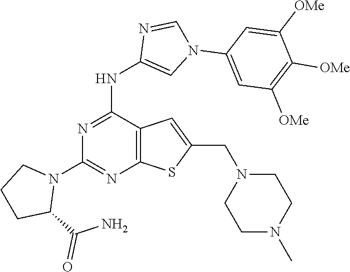 [SMILES: COc1cc(-n2cnc(Nc3nc(N4CCC[C@H]4C(N)=O)nc4sc(CN5CCN(C)CC5)cc34)c2)cc(OC)c1OC]

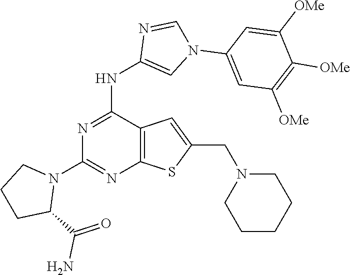 [SMILES: COc1cc(-n2cnc(Nc3nc(N4CCC[C@H]4C(N)=O)nc4sc(CN5CCCCC5)cc34)c2)cc(OC)c1OC]

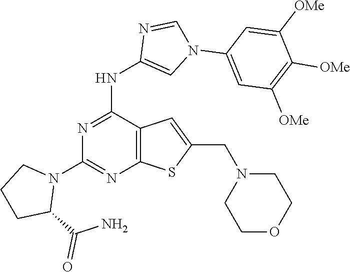 [SMILES: COc1cc(-n2cnc(Nc3nc(N4CCC[C@H]4C(N)=O)nc4sc(CN5CCOCC5)cc34)c2)cc(OC)c1OC]

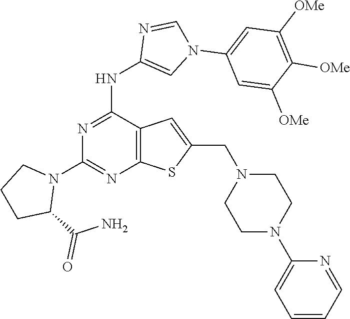 [SMILES: COc1cc(-n2cnc(Nc3nc(N4CCC[C@H]4C(N)=O)nc4sc(CN5CCN(c6ccccn6)CC5)cc34)c2)cc(OC)c1OC]

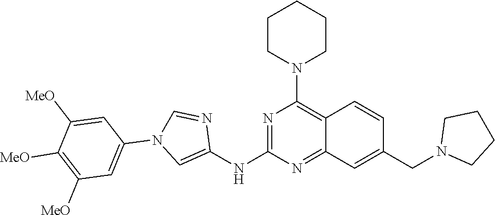 [SMILES: COc1cc(-n2cnc(Nc3nc(N4CCCCC4)c4ccc(CN5CCCC5)cc4n3)c2)cc(OC)c1OC]

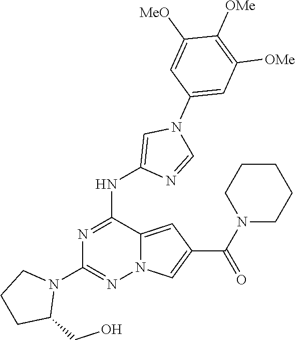 [SMILES: COc1cc(-n2cnc(Nc3nc(N4CCC[C@H]4CO)nn4cc(C(=O)N5CCCCC5)cc34)c2)cc(OC)c1OC]

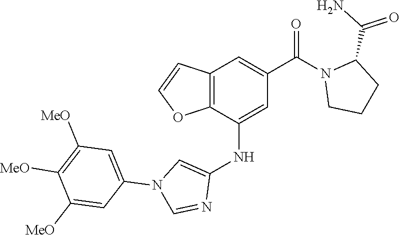 [SMILES: COc1cc(-n2cnc(Nc3cc(C(=O)N4CCC[C@H]4C(N)=O)cc4ccoc34)c2)cc(OC)c1OC]

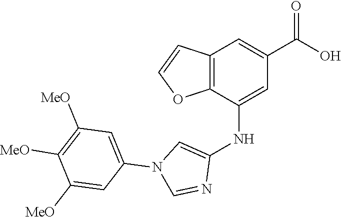 [SMILES: COc1cc(-n2cnc(Nc3cc(C(=O)O)cc4ccoc34)c2)cc(OC)c1OC]

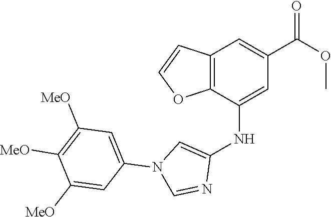 [SMILES: COC(=O)c1cc(Nc2cn(-c3cc(OC)c(OC)c(OC)c3)cn2)c2occc2c1]

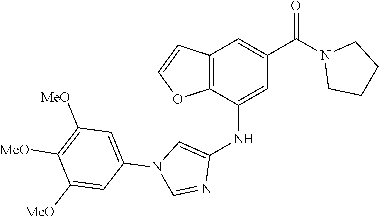 [SMILES: COc1cc(-n2cnc(Nc3cc(C(=O)N4CCCC4)cc4ccoc34)c2)cc(OC)c1OC]

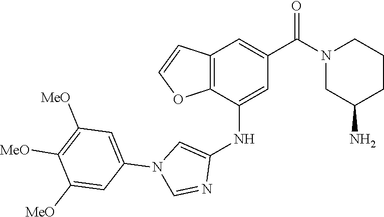 [SMILES: COc1cc(-n2cnc(Nc3cc(C(=O)N4CCC[C@@H](N)C4)cc4ccoc34)c2)cc(OC)c1OC]

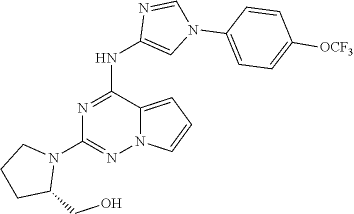 [SMILES: OC[C@@H]1CCCN1c1nc(Nc2cn(-c3ccc(OC(F)(F)F)cc3)cn2)c2cccn2n1]

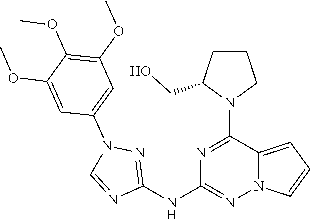 [SMILES: COc1cc(-n2cnc(Nc3nc(N4CCC[C@H]4CO)c4cccn4n3)n2)cc(OC)c1OC]

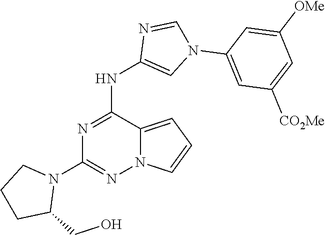 [SMILES: COC(=O)c1cc(OC)cc(-n2cnc(Nc3nc(N4CCC[C@H]4CO)nn4cccc34)c2)c1]

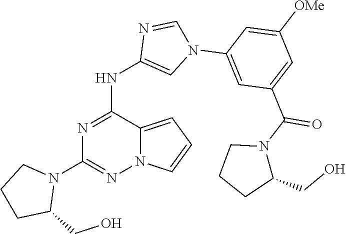 [SMILES: COc1cc(C(=O)N2CCC[C@H]2CO)cc(-n2cnc(Nc3nc(N4CCC[C@H]4CO)nn4cccc34)c2)c1]

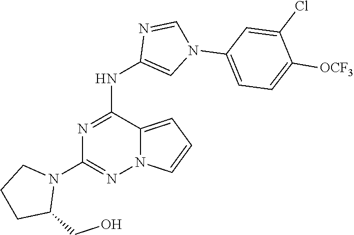 [SMILES: OC[C@@H]1CCCN1c1nc(Nc2cn(-c3ccc(OC(F)(F)F)c(Cl)c3)cn2)c2cccn2n1]

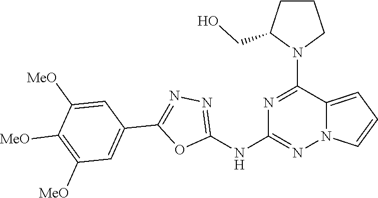 [SMILES: COc1cc(-c2nnc(Nc3nc(N4CCC[C@H]4CO)c4cccn4n3)o2)cc(OC)c1OC]

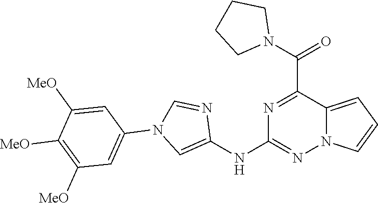 [SMILES: COc1cc(-n2cnc(Nc3nc(C(=O)N4CCCC4)c4cccn4n3)c2)cc(OC)c1OC]

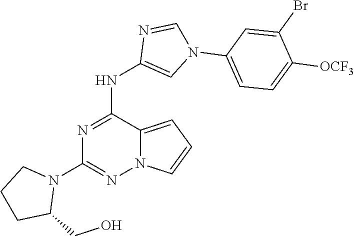 [SMILES: OC[C@@H]1CCCN1c1nc(Nc2cn(-c3ccc(OC(F)(F)F)c(Br)c3)cn2)c2cccn2n1]

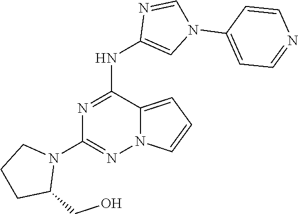 [SMILES: OC[C@@H]1CCCN1c1nc(Nc2cn(-c3ccncc3)cn2)c2cccn2n1]

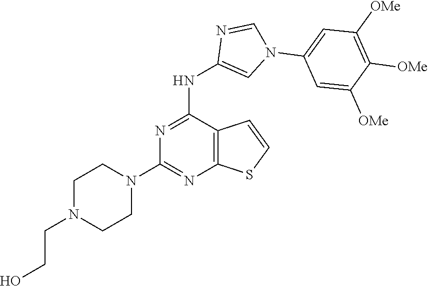 [SMILES: COc1cc(-n2cnc(Nc3nc(N4CCN(CCO)CC4)nc4sccc34)c2)cc(OC)c1OC]

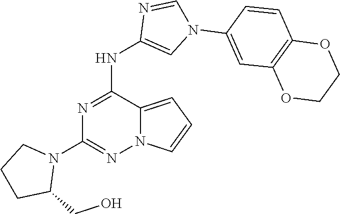 [SMILES: OC[C@@H]1CCCN1c1nc(Nc2cn(-c3ccc4c(c3)OCCO4)cn2)c2cccn2n1]

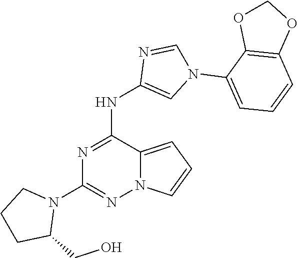 [SMILES: OC[C@@H]1CCCN1c1nc(Nc2cn(-c3cccc4c3OCO4)cn2)c2cccn2n1]

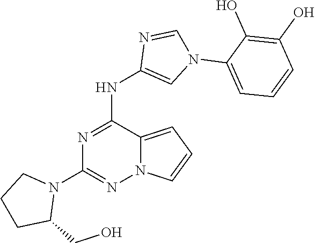 [SMILES: OC[C@@H]1CCCN1c1nc(Nc2cn(-c3cccc(O)c3O)cn2)c2cccn2n1]

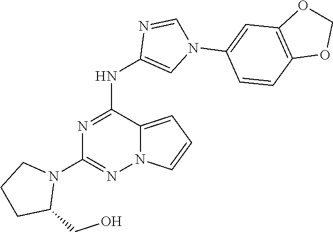 [SMILES: OC[C@@H]1CCCN1c1nc(Nc2cn(-c3ccc4c(c3)OCO4)cn2)c2cccn2n1]

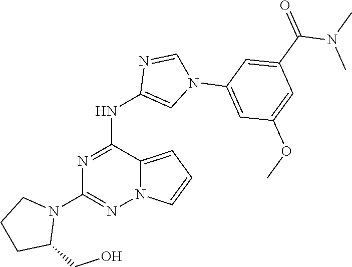 [SMILES: COc1cc(C(=O)N(C)C)cc(-n2cnc(Nc3nc(N4CCC[C@H]4CO)nn4cccc34)c2)c1]

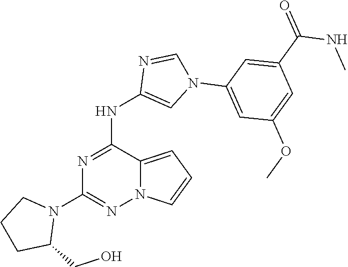 [SMILES: CNC(=O)c1cc(OC)cc(-n2cnc(Nc3nc(N4CCC[C@H]4CO)nn4cccc34)c2)c1]

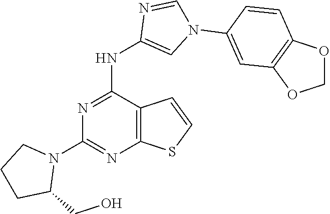 [SMILES: OC[C@@H]1CCCN1c1nc(Nc2cn(-c3ccc4c(c3)OCO4)cn2)c2ccsc2n1]

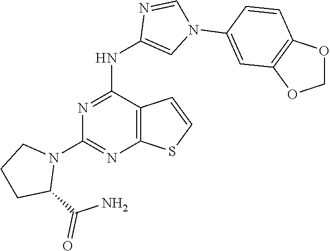 [SMILES: NC(=O)[C@@H]1CCCN1c1nc(Nc2cn(-c3ccc4c(c3)OCO4)cn2)c2ccsc2n1]

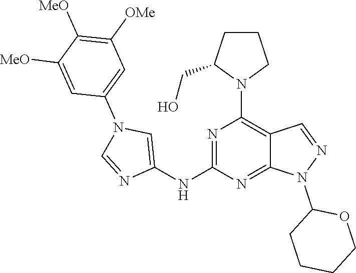 [SMILES: COc1cc(-n2cnc(Nc3nc(N4CCC[C@H]4CO)c4cnn(C5CCCCO5)c4n3)c2)cc(OC)c1OC]

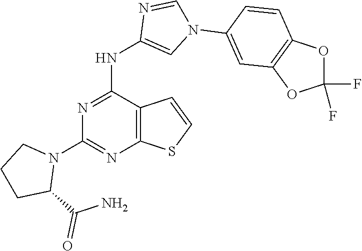 [SMILES: NC(=O)[C@@H]1CCCN1c1nc(Nc2cn(-c3ccc4c(c3)OC(F)(F)O4)cn2)c2ccsc2n1]

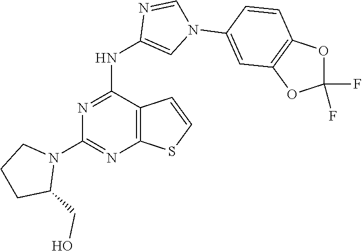 [SMILES: OC[C@@H]1CCCN1c1nc(Nc2cn(-c3ccc4c(c3)OC(F)(F)O4)cn2)c2ccsc2n1]

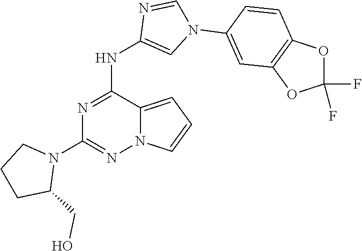 [SMILES: OC[C@@H]1CCCN1c1nc(Nc2cn(-c3ccc4c(c3)OC(F)(F)O4)cn2)c2cccn2n1]

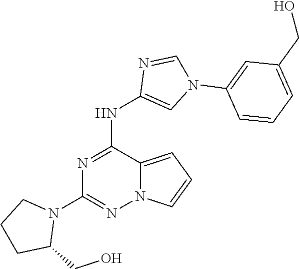 [SMILES: OCc1cccc(-n2cnc(Nc3nc(N4CCC[C@H]4CO)nn4cccc34)c2)c1]

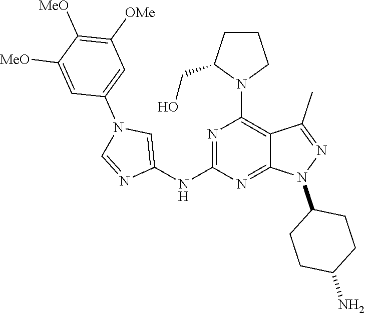

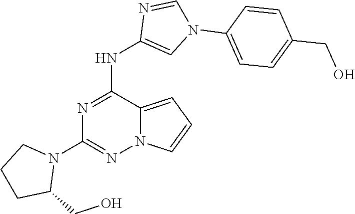 [SMILES: OCc1ccc(-n2cnc(Nc3nc(N4CCC[C@H]4CO)nn4cccc34)c2)cc1]

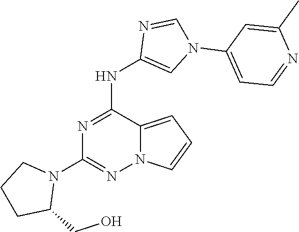 [SMILES: Cc1cc(-n2cnc(Nc3nc(N4CCC[C@H]4CO)nn4cccc34)c2)ccn1]

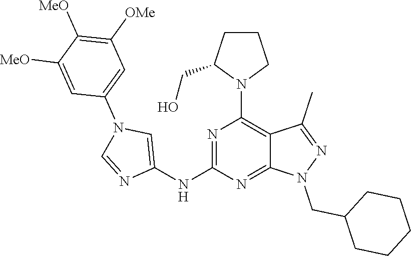 [SMILES: COc1cc(-n2cnc(Nc3nc(N4CCC[C@H]4CO)c4c(C)nn(CC5CCCCC5)c4n3)c2)cc(OC)c1OC]

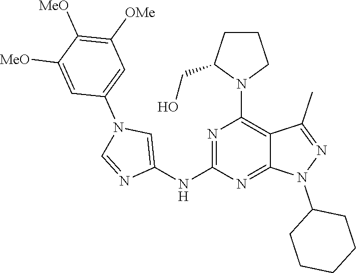 [SMILES: COc1cc(-n2cnc(Nc3nc(N4CCC[C@H]4CO)c4c(C)nn(C5CCCCC5)c4n3)c2)cc(OC)c1OC]

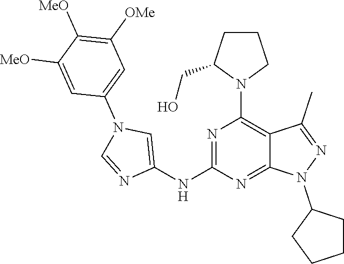 [SMILES: COc1cc(-n2cnc(Nc3nc(N4CCC[C@H]4CO)c4c(C)nn(C5CCCC5)c4n3)c2)cc(OC)c1OC]

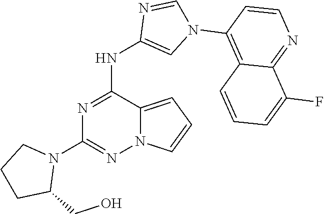 [SMILES: OC[C@@H]1CCCN1c1nc(Nc2cn(-c3ccnc4c(F)cccc34)cn2)c2cccn2n1]

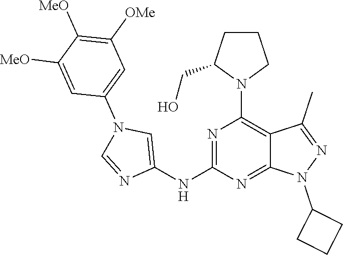 [SMILES: COc1cc(-n2cnc(Nc3nc(N4CCC[C@H]4CO)c4c(C)nn(C5CCC5)c4n3)c2)cc(OC)c1OC]

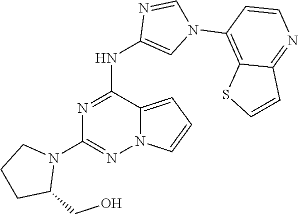 [SMILES: OC[C@@H]1CCCN1c1nc(Nc2cn(-c3ccnc4ccsc34)cn2)c2cccn2n1]

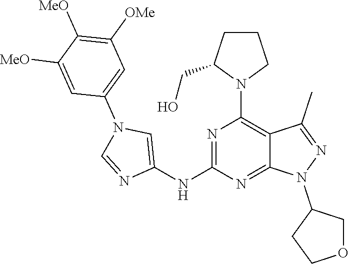 [SMILES: COc1cc(-n2cnc(Nc3nc(N4CCC[C@H]4CO)c4c(C)nn(C5CCOC5)c4n3)c2)cc(OC)c1OC]

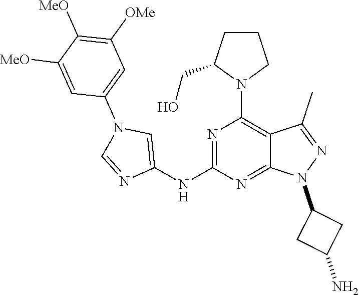

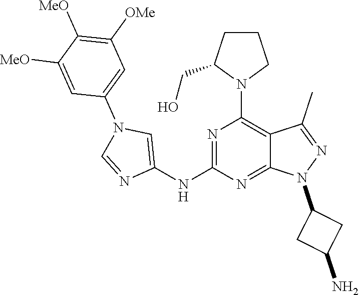

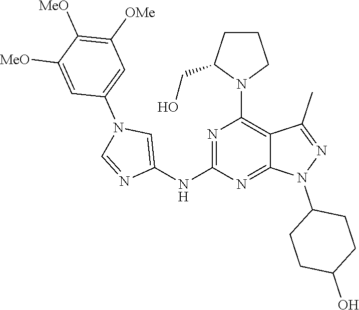 [SMILES: COc1cc(-n2cnc(Nc3nc(N4CCC[C@H]4CO)c4c(C)nn(C5CCC(O)CC5)c4n3)c2)cc(OC)c1OC]

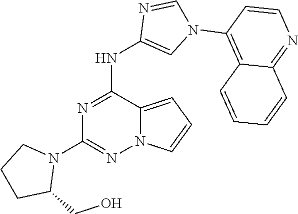 [SMILES: OC[C@@H]1CCCN1c1nc(Nc2cn(-c3ccnc4ccccc34)cn2)c2cccn2n1]

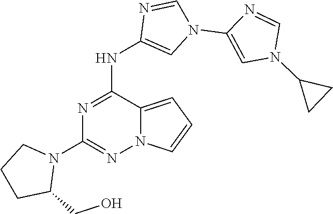 [SMILES: OC[C@@H]1CCCN1c1nc(Nc2cn(-c3cn(C4CC4)cn3)cn2)c2cccn2n1]

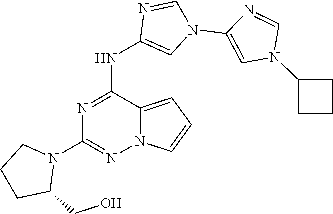 [SMILES: OC[C@@H]1CCCN1c1nc(Nc2cn(-c3cn(C4CCC4)cn3)cn2)c2cccn2n1]

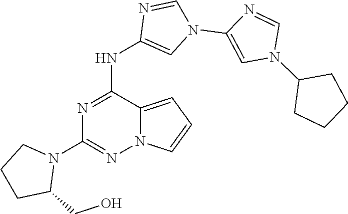 [SMILES: OC[C@@H]1CCCN1c1nc(Nc2cn(-c3cn(C4CCCC4)cn3)cn2)c2cccn2n1]

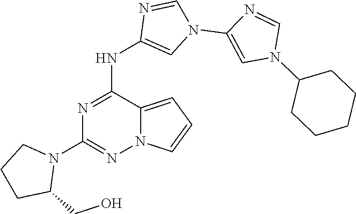 [SMILES: OC[C@@H]1CCCN1c1nc(Nc2cn(-c3cn(C4CCCCC4)cn3)cn2)c2cccn2n1]

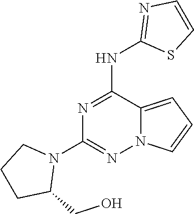 [SMILES: OC[C@@H]1CCCN1c1nc(Nc2nccs2)c2cccn2n1]

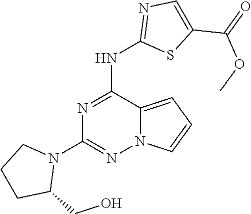 [SMILES: COC(=O)c1cnc(Nc2nc(N3CCC[C@H]3CO)nn3cccc23)s1]

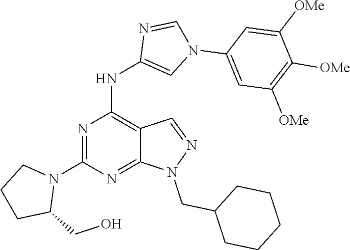 [SMILES: COc1cc(-n2cnc(Nc3nc(N4CCC[C@H]4CO)nc4c3cnn4CC3CCCCC3)c2)cc(OC)c1OC]

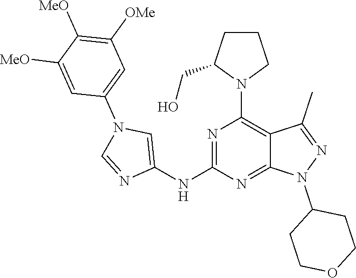 [SMILES: COc1cc(-n2cnc(Nc3nc(N4CCC[C@H]4CO)c4c(C)nn(C5CCOCC5)c4n3)c2)cc(OC)c1OC]

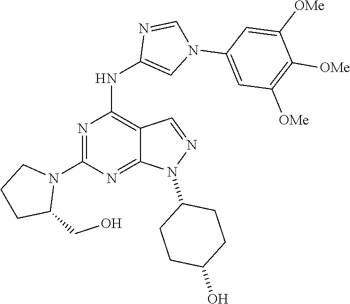

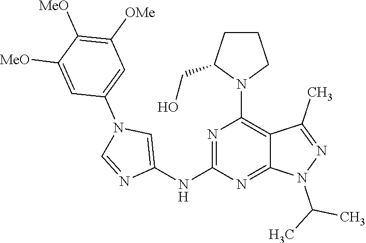 [SMILES: COc1cc(-n2cnc(Nc3nc(N4CCC[C@H]4CO)c4c(C)nn(C(C)C)c4n3)c2)cc(OC)c1OC]

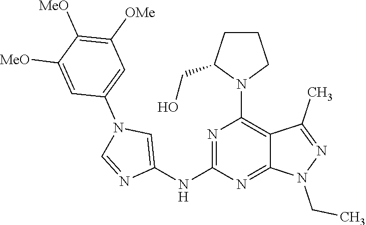 [SMILES: CCn1nc(C)c2c(N3CCC[C@H]3CO)nc(Nc3cn(-c4cc(OC)c(OC)c(OC)c4)cn3)nc21]

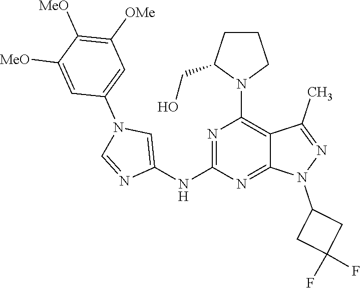 [SMILES: COc1cc(-n2cnc(Nc3nc(N4CCC[C@H]4CO)c4c(C)nn(C5CC(F)(F)C5)c4n3)c2)cc(OC)c1OC]

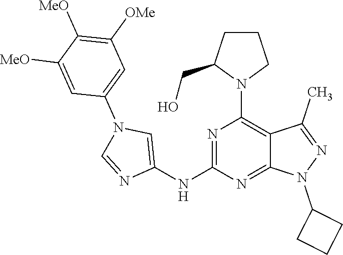 [SMILES: COc1cc(-n2cnc(Nc3nc(N4CCC[C@@H]4CO)c4c(C)nn(C5CCC5)c4n3)c2)cc(OC)c1OC]

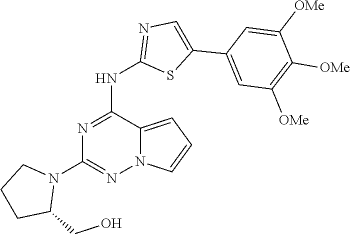 [SMILES: COc1cc(-c2cnc(Nc3nc(N4CCC[C@H]4CO)nn4cccc34)s2)cc(OC)c1OC]

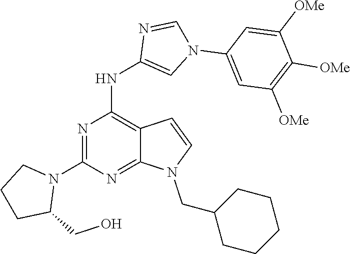 [SMILES: COc1cc(-n2cnc(Nc3nc(N4CCC[C@H]4CO)nc4c3ccn4CC3CCCCC3)c2)cc(OC)c1OC]

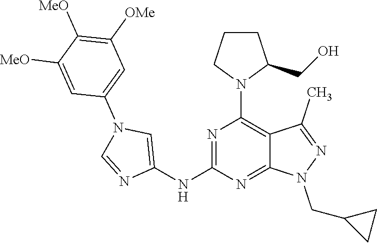 [SMILES: COc1cc(-n2cnc(Nc3nc(N4CCC[C@H]4CO)c4c(C)nn(CC5CC5)c4n3)c2)cc(OC)c1OC]

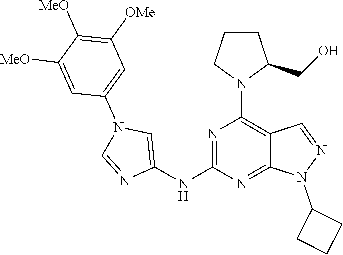 [SMILES: COc1cc(-n2cnc(Nc3nc(N4CCC[C@H]4CO)c4cnn(C5CCC5)c4n3)c2)cc(OC)c1OC]

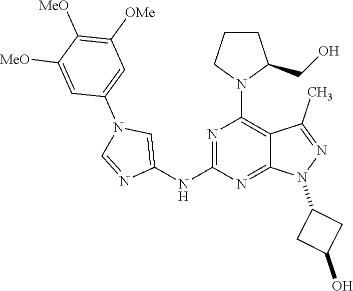

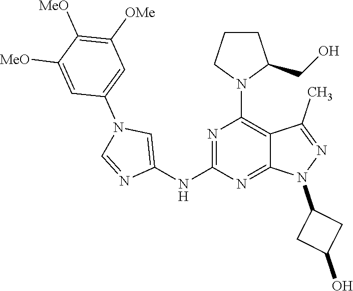

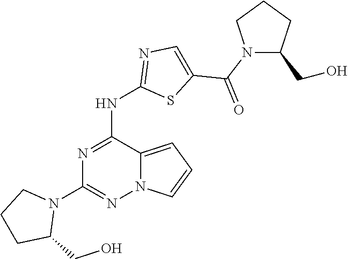 [SMILES: O=C(c1cnc(Nc2nc(N3CCC[C@H]3CO)nn3cccc23)s1)N1CCC[C@H]1CO]

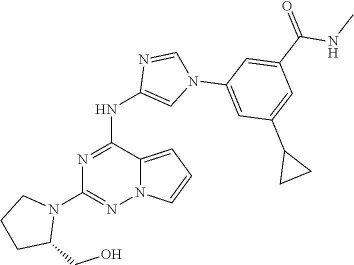 [SMILES: CNC(=O)c1cc(C2CC2)cc(-n2cnc(Nc3nc(N4CCC[C@H]4CO)nn4cccc34)c2)c1]

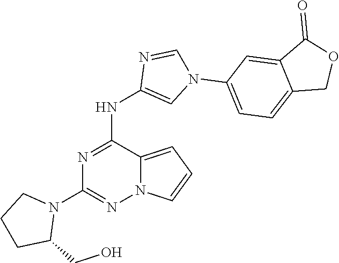 [SMILES: O=C1OCc2ccc(-n3cnc(Nc4nc(N5CCC[C@H]5CO)nn5cccc45)c3)cc21]

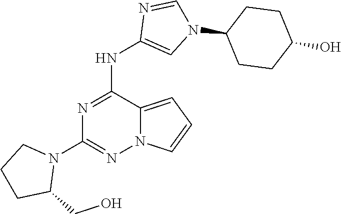 [SMILES: OC[C@@H]1CCCN1c1nc(Nc2cn([C@H]3CC[C@H](O)CC3)cn2)c2cccn2n1]

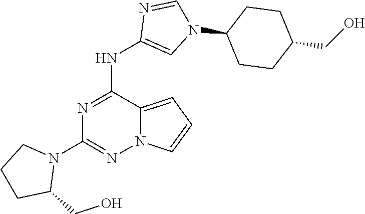 [SMILES: OC[C@@H]1CCCN1c1nc(Nc2cn([C@H]3CC[C@H](CO)CC3)cn2)c2cccn2n1]

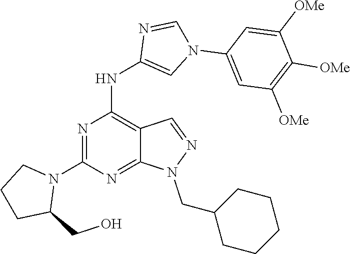 [SMILES: COc1cc(-n2cnc(Nc3nc(N4CCC[C@@H]4CO)nc4c3cnn4CC3CCCCC3)c2)cc(OC)c1OC]

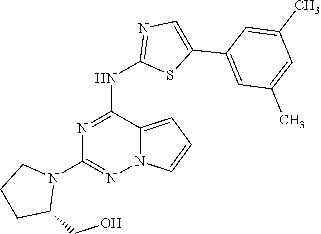 [SMILES: Cc1cc(C)cc(-c2cnc(Nc3nc(N4CCC[C@H]4CO)nn4cccc34)s2)c1]

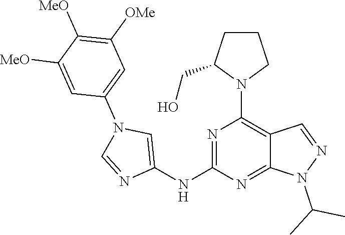 [SMILES: COc1cc(-n2cnc(Nc3nc(N4CCC[C@H]4CO)c4cnn(C(C)C)c4n3)c2)cc(OC)c1OC]

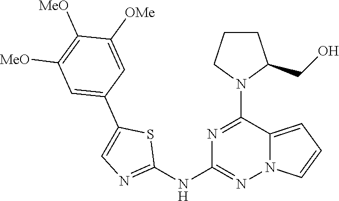 [SMILES: COc1cc(-c2cnc(Nc3nc(N4CCC[C@H]4CO)c4cccn4n3)s2)cc(OC)c1OC]

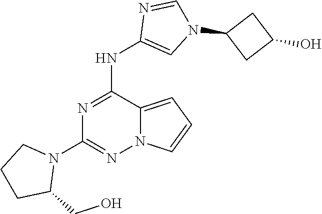 [SMILES: OC[C@@H]1CCCN1c1nc(Nc2cn([C@H]3C[C@H](O)C3)cn2)c2cccn2n1]

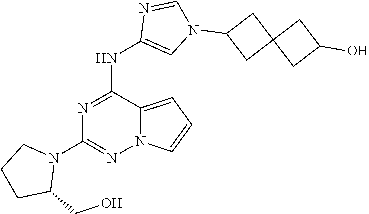 [SMILES: OC[C@@H]1CCCN1c1nc(Nc2cn(C3CC4(CC(O)C4)C3)cn2)c2cccn2n1]

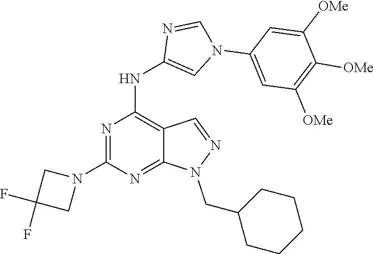 [SMILES: COc1cc(-n2cnc(Nc3nc(N4CC(F)(F)C4)nc4c3cnn4CC3CCCCC3)c2)cc(OC)c1OC]

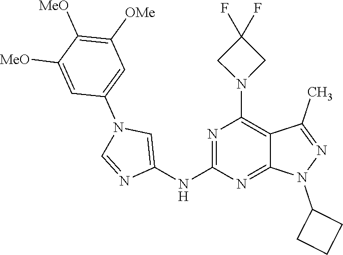 [SMILES: COc1cc(-n2cnc(Nc3nc(N4CC(F)(F)C4)c4c(C)nn(C5CCC5)c4n3)c2)cc(OC)c1OC]

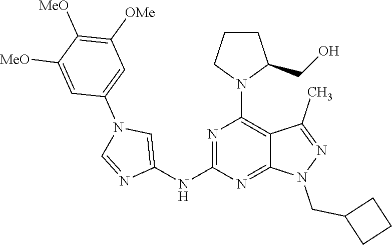 [SMILES: COc1cc(-n2cnc(Nc3nc(N4CCC[C@H]4CO)c4c(C)nn(CC5CCC5)c4n3)c2)cc(OC)c1OC]

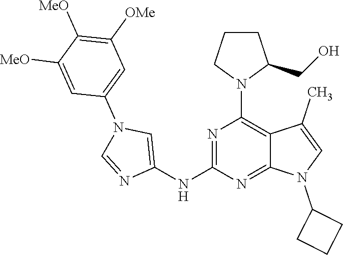 [SMILES: COc1cc(-n2cnc(Nc3nc(N4CCC[C@H]4CO)c4c(C)cn(C5CCC5)c4n3)c2)cc(OC)c1OC]

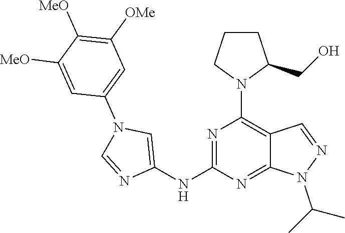 [SMILES: COc1cc(-n2cnc(Nc3nc(N4CCC[C@H]4CO)c4cnn(C(C)C)c4n3)c2)cc(OC)c1OC]

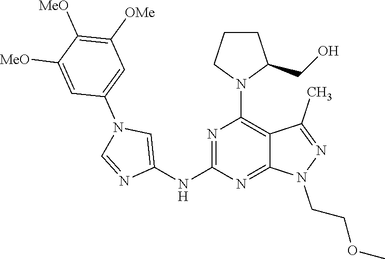 [SMILES: COCCn1nc(C)c2c(N3CCC[C@H]3CO)nc(Nc3cn(-c4cc(OC)c(OC)c(OC)c4)cn3)nc21]

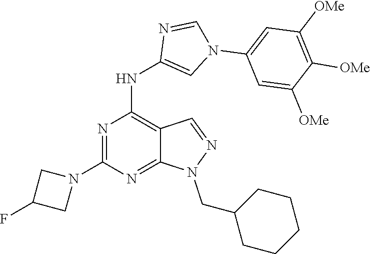 [SMILES: COc1cc(-n2cnc(Nc3nc(N4CC(F)C4)nc4c3cnn4CC3CCCCC3)c2)cc(OC)c1OC]

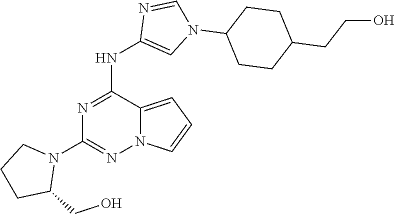 [SMILES: OCCC1CCC(n2cnc(Nc3nc(N4CCC[C@H]4CO)nn4cccc34)c2)CC1]

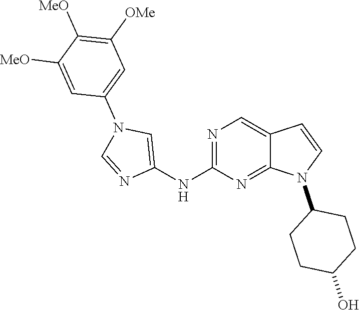

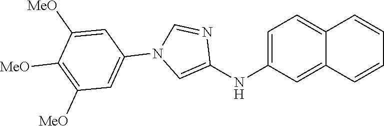 [SMILES: COc1cc(-n2cnc(Nc3ccc4ccccc4c3)c2)cc(OC)c1OC]

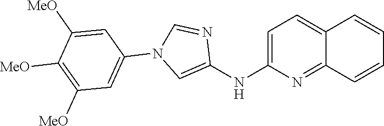 [SMILES: COc1cc(-n2cnc(Nc3ccc4ccccc4n3)c2)cc(OC)c1OC]

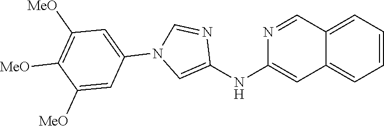 [SMILES: COc1cc(-n2cnc(Nc3cc4ccccc4cn3)c2)cc(OC)c1OC]

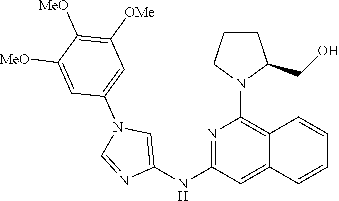 [SMILES: COc1cc(-n2cnc(Nc3cc4ccccc4c(N4CCC[C@H]4CO)n3)c2)cc(OC)c1OC]

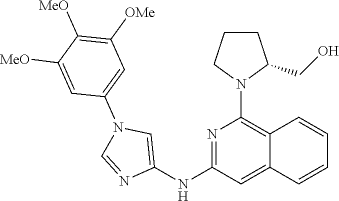 [SMILES: COc1cc(-n2cnc(Nc3cc4ccccc4c(N4CCC[C@@H]4CO)n3)c2)cc(OC)c1OC]

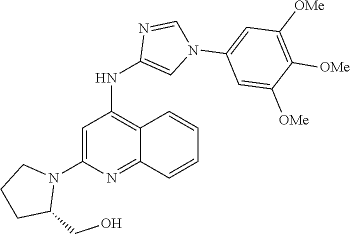 [SMILES: COc1cc(-n2cnc(Nc3cc(N4CCC[C@H]4CO)nc4ccccc34)c2)cc(OC)c1OC]

or a pharmaceutically acceptable salt thereof, and a pharmaceutically acceptable carrier.

In certain embodiments, the oral dosage form is a capsule. In certain embodiments, the oral dosage form is a hard or soft gelatin capsule.

In certain embodiments, the oral dosage form comprises a therapeutically effective amount of a compound of the disclosure (including Compound A).

In certain embodiments, the oral dosage form comprises a therapeutically effective amount of a compound of the disclosure (including Compound A), wherein the therapeutically effective amount is from about 10 mg to about 500 mg per day (such as, but not limited to, about 10, about 20, about 40, about 60, about 80, about 100, about 120, about 140, about 160, about 180, or about 200 mg per day).

In certain embodiments, the oral dosage form comprises a therapeutically effective amount of a compound of the disclosure (including Compound A), wherein the therapeutically effective amount is from about 10 mg to about 500 mg per day (such as, but not limited to, about 10, about 20, about 40, about 60, about 80, about 100, about 120, about 140, about 160, about 180, or about 200 mg per day) and the oral dosage form is a capsule.

In certain embodiments, the oral dosage form comprises a therapeutically effective amount of a compound of the disclosure (including Compound A), wherein the therapeutically effective amount is from about 10 mg to about 500 mg per day (such as, but not limited to, about 10, about 20, about 40, about 60, about 80, about 100, about 120, about 140, about 160, about 180, or about 200 mg per day) and the oral dosage form is a hard or soft gelatin capsule.

In certain embodiments, the oral dosage form comprises a therapeutically effective amount of a compound of the disclosure (including Compound A), wherein the therapeutically effective amount is from about 10 mg to about 250 mg per day (such as, but not limited to, about 10, about 20, about 40, about 60, about 80, about 100, about 120, about 140, about 160, about 180, or about 200 mg per day).

In certain embodiments, the oral dosage form comprises a therapeutically effective amount of a compound of the disclosure (including Compound A), wherein the therapeutically effective amount is from about 10 mg to about 250 mg per day (such as, but not limited to, about 10, about 20, about 40, about 60, about 80, about 100, about 120, about 140, about 160, about 180, or about 200 mg per day) and the oral dosage form is a capsule. In certain embodiments, the oral dosage form comprises a therapeutically effective amount of a compound of the disclosure (including Compound A), wherein the therapeutically effective amount is from about 10 mg to about 250 mg per day and the oral dosage form is a hard or soft gelatin capsule.

Suitable dosage forms, suitable concentrations of a compound of the disclosure in such dosage forms, and suitable doses for administration are described in more detail herein.

In certain embodiments, a compound of the disclosure inhibits wtALK2 and/or ALK2(R206H) activity with a potency (IC₅₀) of less than 100 nM (as described in International Patent Application No. PCT/US2018/037503, which is incorporated by reference for such teaching).

Synthetic methods, characterization data, and assay data for the compounds listed in Table 1 is disclosed in International Patent Application No. PCT/US2018/037503, which is hereby incorporated by reference for such teachings.

### Pharmaceutical Compositions

The disclosure provides pharmaceutical compositions, each comprising one or more compounds of the disclosure, or pharmaceutically acceptable salts thereof, and a pharmaceutically acceptable carrier. In certain embodiments, the pharmaceutical composition comprises a compound of the disclosure, or a pharmaceutically acceptable salt thereof, and a pharmaceutically acceptable carrier. In certain embodiments, the pharmaceutical composition comprises a plurality of compounds of the disclosure, or pharmaceutically acceptable salts thereof, and a pharmaceutically acceptable carrier.

In certain embodiments, a pharmaceutical composition of the disclosure further comprises at least one additional pharmaceutically active agent other than a compound of the disclosure. The at least one additional pharmaceutically active agent can be an agent useful in the treatment of a disease or condition that would be benefitted by inhibition of ALK2 kinase.

Pharmaceutical compositions of the disclosure can be prepared by combining one or more compounds of the disclosure, or pharmaceutically acceptable salts thereof, with a pharmaceutically acceptable carrier and, optionally, one or more additional pharmaceutically active agents.

### Methods of Use

The present disclosure provides compounds, and pharmaceutically acceptable salts thereof, that are useful for treating or preventing a disease or condition whose treatment would benefit from ALK2 kinase inhibition.

In certain aspects, the disclosure provides a method of inhibiting ALK2 kinase in a subject, the method comprising administering to a subject in need thereof a therapeutically effective amount of a compound of the disclosure (e.g., a compound disclosed in Table 1, including Compound A), or a pharmaceutically acceptable salt thereof.

In certain aspects, the disclosure provides a compound of the disclosure (e.g., a compound disclosed in Table 1, including Compound A), or a pharmaceutically acceptable salt thereof, for use as a medicament.

In certain aspects, the disclosure provides methods of treating fibrodysplasia ossificans progressive in a subject, the method comprising the step of administering to a subject in need thereof a therapeutically effective amount of a compound of the disclosure ((e.g., a compound disclosed in Table 1, including Compound A), or a pharmaceutically acceptable salt thereof.

In certain aspects, the disclosure provides methods of treating fibrodysplasia ossificans progressive in a subject, the method comprising the step of administering to a subject in need thereof a therapeutically effective amount of Compound A, or a pharmaceutically acceptable salt thereof.

In certain aspects, the disclosure provides a method of treating cancer in a subject, the method comprising the step of administering to a subject in need thereof a therapeutically effective amount of a compound of the disclosure (e.g., a compound disclosed in Table 1, including Compound A).

In certain aspects, the disclosure provides a method of treating cancer in a subject, the method comprising the step of administering to a subject in need thereof a therapeutically effective amount of Compound A, or a pharmaceutically acceptable salt thereof.

In certain embodiments, the cancer comprises tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcoma, soft tissue sarcomas, osteosarcomas, peripheral primitive neuroectodermal tumors, retinoblastomas, rhabdomyosarcomas, and Wilms tumors.

In certain embodiments, the cancer is a glioma, such as diffuse intrinsic pontine glioma.

In certain aspects, the disclosure provides a method of treating glioma in a subject, the method comprising the step of administering to a subject in need thereof a therapeutically effective amount of a compound of the disclosure (e.g., a compound disclosed in Table 1, including Compound A).

In certain aspects, the disclosure provides a method of treating glioma in a subject, the method comprising the step of administering to a subject in need thereof a therapeutically effective amount of Compound A, or a pharmaceutically acceptable salt thereof.

In certain aspects, the disclosure provides a method of treating diffuse intrinsic pontine glioma in a subject, the method comprising the step of administering to a subject in need thereof a therapeutically effective amount of a compound of the disclosure (e.g., a compound disclosed in Table 1, including Compound A).

In certain aspects, the disclosure provides a method of treating diffuse intrinsic pontine glioma in a subject, the method comprising the step of administering to a subject in need thereof a therapeutically effective amount of Compound A, or a pharmaceutically acceptable salt thereof.

The compounds of the disclosure are useful in treating any disease or condition whose treatment would benefit from ALK2 kinase inhibition, meaning that in such disease or condition it would be desirable to reduce ALK2 kinase activity. For example, it may be desirable to reduce ALK2 kinase activity in the setting of inappropriate activation or hyperactivation of ALK2 kinase, such as in the presence of an activating mutation of ALK2 kinase (including but not limited to the ALK2(R206H) mutation.

In certain aspects, the disclosure provides a method of treating any disease or condition whose treatment would benefit from ALK2 kinase inhibition (meaning that in such disease or condition it would be desirable to reduce ALK2 kinase activity), the method comprising the step of administering to a subject in need thereof a therapeutically effective amount of a compound of the disclosure (e.g., a compound disclosed in Table 1, including Compound A). Such a disease or condition may be characterized by inappropriate activation or hyperactivation of ALK2 kinase, such as in the presence of an activating mutation of ALK2 kinase (including but not limited to the ALK2(R206H) mutation.

In certain aspects, the disclosure provides a method of treating any disease or condition whose treatment would benefit from ALK2 kinase inhibition (meaning that in such disease or condition it would be desirable to reduce ALK2 kinase activity), the method comprising the step of administering to a subject in need thereof a therapeutically effective amount of Compound A, or a pharmaceutically acceptable salt thereof. Such a disease or condition may be characterized by inappropriate activation or hyperactivation of ALK2 kinase, such as in the presence of an activating mutation of ALK2 kinase (including but not limited to the ALK2(R206H) mutation.

In any of the foregoing methods, the administration is oral administration.

In any of the foregoing methods, the administration is oral administration, and the therapeutically effective amount is from about 10 mg to about 500 mg per day (such as, but not limited to, about 10, about 20, about 40, about 60, about 80, about 100, about 120, about 140, about 160, about 180, or about 200 mg per day).

In any of the foregoing methods, the administration is oral administration, and the therapeutically effective amount is from about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day.

In any of the foregoing methods, the administration is oral administration, and the therapeutically effective amount is from about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single oral dosage form.

In any of the foregoing methods, the administration is oral administration, and the therapeutically effective amount is from about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day as a single hard or soft gelatin capsule.

In any of the foregoing methods, the administration is oral administration, and the therapeutically effective amount is from about 10 mg to about 250 mg per day (such as, but not limited to, about 10, about 20, about 40, about 60, about 80, about 100, about 120, about 140, about 160, about 180, or about 200 mg per day).

In any of the foregoing methods, the administration is oral administration, and the therapeutically effective amount is from about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day.

In any of the foregoing methods, the administration is oral administration, and the therapeutically effective amount is from about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single oral dosage form.

In any of the foregoing methods, the administration is oral administration, and the therapeutically effective amount is from about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day as a single hard or soft gelatin capsule.

In any of the foregoing, the length of administration may be from 1 day to 6 months or longer, such as 7 days, 14, days, 21 days, or 28 days. Furthermore, in any of the foregoing, the length of administration may be continuous during the life of the subject.

In a specific aspect, the disclosure provides methods of treating fibrodysplasia ossificans progressive in a subject, the method comprising the step of orally administering to a subject in need thereof a therapeutically effective amount of a compound of the disclosure (e.g., a compound disclosed in Table 1, including Compound A), or a pharmaceutically acceptable salt thereof.

In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day). In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day. In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single oral dosage form. In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single hard or soft gelatin capsule. In any of the foregoing, the length of administration may be from 1 day to 6 months or longer, such as 7 days, 14, days, 21 days, or 28 days. Furthermore, in any of the foregoing, the length of administration may be continuous during the life of the subject.

In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day). In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day. In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single oral dosage form. In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single hard or soft gelatin capsule. In any of the foregoing, the length of administration may be from 1 day to 6 months or longer, such as 7 days, 14, days, 21 days, or 28 days. Furthermore, in any of the foregoing, the length of administration may be continuous during the life of the subject.

In a specific aspect, the disclosure provides methods of treating fibrodysplasia ossificans progressive in a subject, the method comprising the step of orally administering to a subject in need thereof a therapeutically effective amount of Compound A, or a pharmaceutically acceptable salt thereof.

In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day). In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day. In such an aspect, the therapeutically effective amount may be about 10 mg to about 500 mg per day (such as, but not limited to, about 10, about 20, about 40, about 60, about 80, about 100, about 120, about 140, about 160, about 180, or about 200 per day), wherein the therapeutically effective amount is administered 1 time per day in a single oral dosage form. In such an aspect, the therapeutically effective amount may be about 10 mg to about 500 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single hard or soft gelatin capsule. In any of the foregoing, the length of administration may be from 1 day to 6 months or longer, such as 7 days, 14, days, 21 days, or 28 days. Furthermore, in any of the foregoing, the length of administration may be continuous during the life of the subject.

In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day). In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day. In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single oral dosage form. In such an aspect, the therapeutically effective amount may be about 10 mg to about 250 mg per day (such as, but not limited to, about 10, about 20, about 40, about 60, about 80, about 100, about 120, about 140, about 160, about 180, or about 200 mg per day), wherein the therapeutically effective amount is administered 1 time per day in a single hard or soft gelatin capsule. In any of the foregoing, the length of administration may be from 1 day to 6 months or longer, such as 7 days, 14, days, 21 days, or 28 days. Furthermore, in any of the foregoing, the length of administration may be continuous during the life of the subject.

### Formulations, Routes of Administration, and Dosing

The compounds of the disclosure, and pharmaceutically acceptable salts thereof, can be formulated as pharmaceutical compositions and administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the disclosure. In certain embodiment, a pharmaceutical composition comprising a compounds of the disclosure, or pharmaceutically acceptable salts thereof, is administered to a mammalian host, such as a human patient, via oral administration.

Thus, the present compounds or pharmaceutically acceptable salts thereof may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound (i.e., a compound of the disclosure or a pharmaceutically acceptable salt thereof) may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2% to about 60% of the weight of a given unit dosage form. The amount of active compound in such therapeutically useful compositions is such that an effective dosage level will be obtained.

The tablets, troches, pills, capsules, and the like may also contain the following diluents and carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any pharmaceutical composition or unit dosage form should be pharmaceutically acceptable and substantially nontoxic in the amounts employed. In addition, the pharmaceutical composition may be a sustained-release composition or device.

A pharmaceutical composition of the disclosure may also be administered intravenously, intramuscularly, or intraperitoneally by infusion or injection. Pharmaceutical composition comprising a compound of the disclosure, or pharmaceutically acceptable salts thereof, can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical compositions suitable for injection or infusion can include sterile aqueous solutions or dispersions or sterile powders comprising a compound of the disclosure, or pharmaceutically acceptable salts thereof, which are adapted for the extemporaneous preparation of sterile injectable or infusible solutions or dispersions, optionally encapsulated in liposomes. In all cases, the ultimate pharmaceutical composition should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Pharmaceutical compositions in the form of sterile injectable solutions are prepared by incorporating a compound of the disclosure, or pharmaceutically acceptable salts thereof, in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active compound plus any additional desired ingredient present in the previously sterile-filtered solutions.

For topical administration, a compound of the disclosure, or pharmaceutically acceptable salts thereof, may be applied in pure form, i.e., when they are liquids. However, it will generally be desirable to administer them to the skin as pharmaceutical compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds or pharmaceutically acceptable salts thereof can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the disclosure, or pharmaceutically acceptable salts thereof, to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the compounds of the disclosure, or pharmaceutically acceptable salts thereof, can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of the compound, or pharmaceutically acceptable salt thereof, required for use in treatment will vary not only with the particular compound or salt selected but also with the route of administration, the nature of the condition being treated, and the age and condition of the patient and will be ultimately at the discretion of the attendant physician or clinician.

In general, however, a suitable dose (i.e., a therapeutically effective amount) will be in the range of from about 0.01 to about 100 mg/kg body weight of the recipient per day, e.g., from about 0.04 to about 50 mg/kg of body weight per day, from about 0.05 to about 40 mg per kilogram of body weight per day, from about of 0.06 to about 30 mg/kg of body weight per day, from about 0.06 to about 20 mg/kg of body weight per day, or from about 0.07 to about 10 mg/kg of body weight per day. In a specific embodiment, a suitable dose will be in the range of from about 0.01 to 10 mg/kg of body weight per day, e.g., from about 0.04 to about 8 mg/kg of body weight per day, from about 0.05 to about 7 mg per kilogram of body weight per day, from about of 0.06 to about 6 mg/kg of body weight per day, from about 0.06 to about 5 mg/kg of body weight per day, or from about 0.07 to about 4 mg/kg of body weight per day. In another specific embodiment, a suitable dose will be in the range of from about 0.04 to 5 mg/kg of body weight per day, e.g., from about 0.05 to about 4 mg/kg of body weight per day, from about 0.06 to about 3 mg per kilogram of body weight per day, from about of 0.07 to about 3 mg/kg of body weight per day, from about 0.08 to about 3 mg/kg of body weight per day, from about 0.09 to about 3 mg/kg of body weight per day, or from about 0.1 to about 3 mg/kg of body weight per day. In yet another specific embodiment, a suitable dose will be in the range of from about 0.1 to 4 mg/kg of body weight per day, e.g., from about 0.2 to about 3 mg/kg of body weight per day, from about 0.2 to about 2.5 mg/kg of body weight per day, from about 0.2 to about 2 mg/kg of body weight per day, from about 0.2 to about 1.5 mg/kg of body weight per day, from about 0.2 to about 1 mg/kg of body weight per day, about 1 mg/kg of body weight per day, about 1.5 mg/kg of body weight per day, about 2.0 mg/kg of body weight per day, about 2.5 mg/kg of body weight per day, about 3.0 mg/kg of body weight per day, about 3.5 mg/kg of body weight per day, or about 4 mg/kg of body weight per day.

In one embodiment, a suitable dose range (i.e., a therapeutically effective amount) will be the administration of about 10 mg to about 250 mg of a compound of the disclosure (including Compound A), or a pharmaceutically acceptable salt thereof, per day to a subject, such as about 10 mg to about 225 mg per day, about 10 mg to about 200 mg per day, about 10 mg to about 175 mg per day, about 10 mg to about 150 mg per day, about 10 mg to about 140 mg per day, about 10 mg to about 130 mg per day, about 10 mg to about 120 mg per day, about 10 mg to about 110 mg per day, about 10 mg to about 100 mg per day, about 10 mg to about 90 mg per day, about 10 mg to about 80 mg per day, about 10 mg to about 70 mg per day, about 10 mg to about 60 mg per day, about 10 mg to about 50 mg per day, about 10 mg to about 40 mg per day, about 10 mg to about 30 mg per day, about 10 mg to about 20 mg per day, or about 10 mg to about 15 mg per day.

A compound of the disclosure (including Compound A), or pharmaceutically acceptable salts thereof, can be conveniently formulated in unit dosage forms, for example, containing about 10 to about 1000 mg, about 10 to about 750 mg, about 50 to about 500 mg, about 10 to about 250 mg, about 10 mg to about 225 mg, about 10 mg to about 200 mg, about 10 mg to about 175 mg, about 10 mg to about 150 mg, about 10 mg to about 140 mg, about 10 mg to about 130 mg, about 10 mg to about 120 mg, about 10 mg to about 110 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, or about 10 mg to about 15 mg of a compound of the disclosure, or a pharmaceutically acceptable salt thereof, per unit dosage form. In one embodiment, the disclosure provides a composition comprising a compound of the disclosure, or a pharmaceutically acceptable salt thereof, formulated in such a unit dosage form. The desired dose may conveniently be presented in a single dose or as divided doses to be administered at appropriate intervals, for example, as two, three, four or more sub-doses per day. The sub-dose itself may be further divided, e.g., into a number of discrete loosely spaced administrations.

Compounds of the disclosure, or pharmaceutically acceptable salts thereof, can also be administered in combination with other therapeutic agents, for example, other agents that are useful for treating or preventing a disease or condition whose treatment would benefit from ALK2 kinase inhibition.

Other delivery systems can include time-release, delayed release, or sustained release delivery systems such as are well-known in the art. Such systems can avoid repeated administrations of the active compound, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active compound for at least 30 days, and preferably 60 days.

In certain embodiments, a compound of the disclosure, or pharmaceutically acceptable salt thereof, is formulated for intraocular administration, for example direct injection or insertion within or in association with an intraocular medical device.

The compounds of the disclosure, or pharmaceutically acceptable salts thereof, may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the disclosure, or pharmaceutically acceptable salts thereof, to the region of a body which has been treated by interventional technique.

In exemplary embodiments, a compound of the disclosure, or pharmaceutically acceptable salt thereof, may be deposited within a medical device, such as a stent, and delivered to the treatment site for treatment of a portion of the body.

Stents have been used as delivery vehicles for therapeutic agents (i.e., drugs) Intravascular stents are generally permanently implanted in coronary or peripheral vessels. Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz), U.S. Pat. No. 5,419,760 (Narciso, Jr.), U.S. Pat. No. 5,429,634 (Narciso, Jr.), and in International Patent Application Nos. WO 91/12779 (Medtronic, Inc.) and WO 90/13332 (Cedars-Sanai Medical Center), for example.

The term “deposited” means that the active compound is coated, adsorbed, placed, or otherwise incorporated into the device by methods known in the art. For example, the active compound may be embedded and released from within (“matrix type”) or surrounded by and released through (“reservoir type”) polymer materials that coat or span the medical device. In the latter example, the active compound may be entrapped within the polymer materials or coupled to the polymer materials using one or more the techniques for generating such materials known in the art. In other formulations, the active compound may be linked to the surface of the medical device without the need for a coating, for example by means of detachable bonds, and release with time or can be removed by active mechanical or chemical processes. In other formulations, the active compound may be in a permanently immobilized form that presents the active compound at the implantation site.

In certain embodiments, the active compound may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is suitable for this embodiment because, unlike a biostable polymer, it will typically not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(d-lactic acid), poly(l-lactic acid), poly(d, l-lactic acid), poly(d, l-lactide) (PLA), poly (l-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable polymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; pyran copolymer; polyhydroxy-propyl-methacrylamide-phenol; polyhydroxyethyl-aspartamide-phenol; polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers and semipermeable polymer matrices may be formed into shaped articles, such as valves, stents, tubing, prostheses and the like.

In certain embodiments of the disclosure, the compound of the disclosure, or pharmaceutically acceptable salt thereof, is coupled to a polymer or semipermeable polymer matrix that is formed as a stent or stent-graft device.

Typically, polymers are applied to the surface of an implantable device by spin coating, dipping, or spraying Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the disclosure, the compound of the disclosure, or pharmaceutically acceptable salt thereof, is formulated for release from the polymer coating into the environment in which the medical device is placed. Preferably, the active compound is released in a controlled manner over an extended timeframe (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the active compound from the polymer coating can be controlled. For example, the diffusion coefficient of the one or more polymer coatings can be modulated to control the release of the active compound from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the active compound from the polymer coating). Yet another embodiment of the disclosure includes a device having a plurality of polymer coatings, each having a plurality of diffusion coefficients. In such embodiments of the disclosure, the release of the active compound from the polymer coating can be modulated by the plurality of polymer coatings.

In yet another embodiment of the disclosure, the release of the active compound from the polymer coating is controlled by modulating one or more of the properties of the polymer composition, such as the presence of one or more endogenous or exogenous compounds, or alternatively, the pH of the polymer composition. For example, certain polymer compositions can be designed to release an active compound in response to a decrease in the pH of the polymer composition.

### Kits

The disclosure also provides a kit, comprising a compound of the disclosure, or a pharmaceutically acceptable salt thereof, and at least one additional component including, but not limited to, an additional therapeutic agent (other than a compound of the disclosure or pharmaceutically acceptable salt thereof), packaging material, and instructions for administering the compound of the disclosure or the pharmaceutically acceptable salt thereof (and the other therapeutic agent or agents if included) to a mammal to treat or prevent a disease or condition that would benefit from ALK2 inhibition. In one embodiment, the mammal is a human. In another embodiment, the mammal is a human and the disease or condition is FOP. In another embodiment, the mammal is a human, the disease or condition is FOP, and the instructions provide for the oral administration of a compound of the disclosure, or a pharmaceutically acceptable salt thereof, at a dose of about 10 mg to about 250 mg per day to the human subject. In another embodiment, the mammal is a human, the disease or condition is FOP, and the instructions provide for the oral administration of a compound of the disclosure, or a pharmaceutically acceptable salt thereof, at a dose of about 10 mg to about 250 mg per day to the human subject, wherein the dose is administered 1 time per day. In another embodiment, the mammal is a human, the disease or condition is FOP, and the instructions provide for the oral administration of a compound of the disclosure, or a pharmaceutically acceptable salt thereof, at a dose of about 10 mg to about 250 mg per day to the human subject, wherein the dose is administered 1 time per day in a single oral dosage form (such as, but not limited to, a hard or soft gelatin capsule).

It will be understood by one of ordinary skill in the relevant arts that other suitable modifications and adaptations to the compositions and methods described herein are readily apparent from the description of the disclosure contained herein in view of information known to the ordinarily skilled artisan, and may be made without departing from the scope of the disclosure or any embodiment thereof.

### EXAMPLES

### Example 1—Phase 1 Clinical Study

A Phase 1, randomized, double-blind, dose-ranging study in healthy subjects is currently ongoing to evaluate the safety, tolerability, and PK of Compound A in Part 1 (single ascending dose [SAD]) and Part 2 (multiple ascending dose [MAD]) of the study.

The primary objective of Part 1 and Part 2 of this study is: (1) to evaluate the safety and tolerability of SAD (Part 1) and MAD (Part 2) of Compound A in healthy subjects.

The secondary objectives of Part 1 and Part 2 of this study are: (1) to characterize the plasma and urine PK profile of SAD (Part 1) and MAD (Part 2) of Compound A in healthy subjects; (2) to evaluate the effect of a high-fat meal on the single-dose PK of Compound A in a pilot evaluation in healthy subjects; and (3) to evaluate the effect of Compound A on the ECG intervals of interest and blood pressure.

The primary outcome measure of Part 1 and Part 2 will be: (1) measurement of the safety and tolerability by subject incidence of graded treatment-emergent adverse events (TEAEs), laboratory abnormalities, and changes to vital signs, ECGs, cardiac telemetry, and physical examination findings. The secondary outcome measures of Part 1 and Part 2 will be: (1) PK parameters for Compound A after SAD administration and MAD administration in healthy subjects; (2) dose proportionality of Compound A; (3) accumulation ratio of Compound A (Part 2); (4) change from baseline in standard ECG intervals of interest (QTc, PR, QRS, RR, etc.); and (5) change from baseline in systolic and diastolic BP.

Healthy subjects have been selected for the clinical study of Compound A to minimize variability in factors that may affect the assessment of safety, tolerability, and PK. Healthy subjects will be administered Compound A in Part 1 (SAD) and Part 2 (MAD) of the study. FOP affects both males and females, and it is therefore important to study PK in healthy subjects who are both male and female.

### Inclusion Criteria and Exclusion Criteria

Subjects must meet all of the following inclusion criteria to be eligible for participation in Part 1 and Part 2 of the study: (1) able to provide written, informed consent; (2) body mass index (BMI) of 18.0 to 32.0 kg/m²; (3) creatinine clearance (CLCR) of at least 80 mL/min as measured by the Cockcroft-Gault equation; (4) males and non-pregnant, non-lactating females age 22 to 55 years; (5) subjects must meet the contraception requirements as follows: (i) female subjects of childbearing potential must use a highly effective method of contraception to avoid pregnancy throughout the duration of the study and for 30 days after the last dose of study drug; (ii) female subjects of childbearing potential must have a negative serum or urine pregnancy test prior to the start of study drug; (iii) male subjects with female partners of childbearing potential must agree to use a highly effective method of contraception to avoid pregnancy throughout the duration of the study and for 90 days after the last dose of study drug; and (6) in the opinion of the Investigator, the subject is expected to adequately comply with all required study procedures and restrictions for the duration of the study.

As used in the description of inclusion criteria and/or exclusion criteria a “highly effective method of contraception” means a combined (estrogen and progestogen containing) hormonal contraception (oral, intravaginal, or transdermal), progestogen-only hormonal contraception associated with inhibition of ovulation (oral, injectable, or implantable), intrauterine device (IUD), intrauterine hormone-releasing system (IUS), bilateral tubal occlusion or bilateral tubal ligation, vasectomized partner or sexual abstinence (defined as refraining from heterosexual intercourse during the entire period of risk associated with the study treatments; true abstinence is acceptable when it is in line with the subject's preferred and usual lifestyle). If a subject is usually not sexually active but becomes active, the subject and partner, must meet the requirements listed above.

As used in the description of inclusion criteria and/or exclusion criteria “female subjects of childbearing potential” includes any female who has experienced menarche and who has not undergone successful surgical sterilization (hysterectomy, bilateral tubal ligation, or bilateral oophorectomy) or is not postmenopausal.

As used in the description of inclusion criteria and/or exclusion criteria “postmenopausal” is defined as no menses for ≥12 months without another cause and a documented serum FSH level in the postmenopausal range

Subjects must not meet any of the following exclusion criteria to be eligible for participation in Part 1 and Part 2 of the study: (1) any clinically significant medical or psychiatric condition or medical history that, in the opinion of the investigator or sponsor, would interfere with the subject's ability to participate in the study or increase the risk of participation for that subject. Subjects with the following conditions should be excluded from the study: (i) underlying lung disease (including history of obstructive lung disease, asthma, current respiratory symptoms of breathlessness, cough and wheeze; tobacco use within the last year); (ii) history of liver disease; (iii) history of significant hematologic disorder (e.g., bleeding disorders, anemia, thrombocytopenia, leukopenia, lymphoproliferative disorders, etc.) or active bleeding; (iv) acute or chronic gastrointestinal illness or inflammatory bowel disease; or (v) history of pancreatic disease; (2) for subjects enrolling in Part 1, Cohort 3, which will evaluate the effect of food on the PK of Compound A, a medical history of cholecystectomy or gall stones is exclusionary; (3) clinically significant abnormal ECG at the screening visit (including, but is not limited to, a QTcF >450 msec in males and >460 msec in females, or ventricular and/or atrial premature contractions that are more frequent than occasional, and/or as couplets or higher in grouping); (4) subjects with a systolic BP outside the range of 90 to 140 mmHg or diastolic BP outside the range of 50 to 90 mmHg; (5) any clinically significant history of angina, known coronary artery disease, myocardial infarction, syncope, clinically significant cardiac arrhythmias, left ventricular hypertrophy, cardiomyopathy, aortic stenosis, or any other cardiovascular abnormality; (6) known family history of sudden cardiac death in a first degree relative; (7) history of or current implanted defibrillator or pacemaker; (8) any laboratory parameter at screening that, in the opinion of the investigator, is clinically significant and relevant for this study, including clinically significant electrolyte disturbances (e.g., hyper- or hypo-kalemia, -calcemia or -magnesemia), ALT and AST must be no greater than the ULN (upper limit of normal), and any abnormality in a hematologic parameter must be no greater than Grade 1; (9) clinically significant finding on screening or baseline urinalysis: (10) current participation in any other investigational drug study or participation in an investigational drug study within 30 days prior to the screening visit; (11) current use, or use of any over-the-counter medication, vitamins, or herbal products within 14 days prior to Day 1 (other than up to 2 grams per day acetaminophen); (12) current use of or use of any prescribed drug in the 14 days before Day 1, with the exception of any allowed contraceptives; (13) use of a medication or herbal product that is clinically known to inhibit or induce metabolic enzymes or transporters within 30 days prior to Day 1; (14) history of alcohol or drug abuse within the last 12 months prior to the screening visit, or current evidence of substance dependence or abuse as self-reported alcoholic intake >3 units of alcohol/day: (15) regular alcohol consumption of males of >21 units per week and for females of >14 units per week, where a unit is defined as ½ pint of beer, 25 mL of 40% spirit, and 1.5 units is equivalent to a 125 mL glass of wine: (16) current smokers and those who have smoked within the last 12 months. This includes the use of cigarettes, e-cigarettes and nicotine replacement products; (17) a positive urine cotinine test; (18) positive drugs of abuse screen; (19) positive serology for human immunodeficiency virus (HIV) or active infection with hepatitis B virus (HBV) or hepatitis C virus (HCV); (20) pregnant, planning to become pregnant, or having been pregnant within 90 days prior to Day 1, or lactating; (21) donation or loss of >400 mL of blood within the 3 months prior to dosing; (22) history of severe hypersensitivity to any medicinal product, which was associated with swelling, severe rash requiring treatment/hospitalization, or anaphylaxis; (23) employment by the study site, or an immediate family relationship to either a study site employees or a sponsor employee; or (24) lack of suitable veins.

In addition, to be eligible for Part 1 and Part 2 of the study, subjects must abide by the following additional restrictions. (1) subjects will abstain from all methylxanthine-containing foods and beverages (caffeine, theobromine, or theophylline in coffee, tea, chocolate, colas or energy drinks) from Day −1 until 24 hours following the last dose of study drug; (2) subjects will abstain from alcohol consumption for 2 days prior to dosing through the last follow-up visit; (3) subjects will be asked to avoid strenuous exercise from 7 days prior to Day 1 through the last follow-up visit: (4) subjects will be asked to abstain from all bergamotin-containing fruits and fruit juices (e.g., Seville oranges, grapefruit, grapefruit juice, pomelos, marmalade) from 7 days prior to Day −1 through 24 hours following the last dose of study drug; (5) subjects will be asked to refrain from eating poppy seeds from 2 days prior to Day 1 through the last dose to be administered in Part 1 and through the first dose in Part 2; (6) subjects will be kept inside away from strong sunlight while in the clinical unit and will be advised to avoid exposure to strong sunlight and to use UV-blocking creams on sunny days from discharge until 21 days after the last dose of Compound A; and (7) male subjects must abstain from sperm donation for a period of 90 days after discharge from the study unit.

### Part 1

Four sequential SAD cohorts were dosed. For cohorts 1, 2, and 5 of Part 1, eight subjects were treated with a single dose of study drug per dose cohort (6 subjects per cohort received Compound A, and 2 subjects per cohort received matching placebo) administered orally. For cohort 4 of Part 1, nine subjects were treated with a single dose of study drug per dose cohort (7 subjects per cohort received Compound A, and 2 subjects per cohort received matching placebo) administered orally. The study drug (Compound A or placebo) was provided as size 0 hard gelatin capsules. The 4 dose cohorts were as follows:
Part 1, Cohort 1: 5 mg Compound A or placebo orally, ×1 dose;
Part 1, Cohort 2: 10 mg Compound A or placebo orally, ×1 dose;
Part 1, Cohort 4, Period 1: 15 mg Compound A or placebo orally, ×1 dose (fasted);
Part 1, Cohort 4, Period 2: 15 mg Compound A or placebo orally, ×1 dose (administration after a high fat meal); dosing in Period 2 was separated from dosing in Period 1 by a washout period as described herein);
Part 1, Cohort 5: 25 mg Compound A or placebo orally, ×1 dose.

Subjects in Part 1 were treated with a single oral dose of study drug (Compound A or placebo) per dose cohort. Escalation to the next higher dose level in Part 1 occurred only after satisfactory review of clinical safety and pharmacokinetic data. Subjects in Part 1 Cohort 4 initially received a single dose of study drug under fasting conditions; after an adequate washout period (the longer of 7 days or at least 5 times the terminal elimination half-life, t^{1/2}, of Compound A), all subjects in Part 1 Cohort 4 were given a second dose after a high-fat meal. Subjects received the same study drug and dose (Compound A or placebo) in both dosing periods.

For each cohort of Part 1, the daily dose of study drug was administered as a single dose (1 time daily or QD). In Part 1 of the study, following a screening period to determine eligibility, subjects in each cohort entered the Phase 1 clinical research unit (CRU) on the afternoon of Day −1. Study eligibility was re-confirmed upon admission to the CRU, and subjects remained in the CRU overnight prior to dosing on Day 1. Following Day 1, subjects remained in the CRU for an additional 72 hours post-dose (i.e., discharge on Day 4) to enable collection of plasma and urine for PK samples and other study assessments. Subjects were discharged from the CRU following plasma PK sample collection on Day 4 and returned on Day 16 (+2 days) for a final follow-up visit.

Table 1 shows the number of subjects in each analysis population for Part 1. Overall, of the 33 subjects randomized, all 33 were treated with at least one dose of study drug and were included in the safety population. The safety population was used for all analyses of demographics and safety. The PK population included all subjects who received at least 1 dose of study drug and provided an adequate number of blood samples for the determination of at least 1 PK parameter. One subject in cohort 4 completed the fasted evaluation but not the fed evaluation and was replaced with another subject who completed only the fed evaluation; both subjects were included in the PK population. An additional subject in cohort 4 was randomized to receive Compound A but did not achieve quantifiable plasma concentrations in either the fasted or fed evaluations and was therefore excluded from the PK population. The PK population was used for all PK summaries and analyses.

The PD ECG population for QTcF analysis included all subjects who received at least 1 dose of study drug and for whom at least 1 pre- and post-dose QTcF result was obtained from continuous Holter ECG extraction. The PD ECG population was used for all analyses of QTcF pharmacological effect and for correlation with PK parameters or BCX9250 concentrations. The PD BP population for analysis of changes in systolic and diastolic BP included all subjects who received at least 1 dose of study drug and for whom at least 1 pre- and post-dose BP measurement was obtained. The PD BP population was used for correlation of changes in BP with BCX9250 concentrations and/or PK parameters.

TABLE 1
Analysis Populations (Part 1)

| | | | 15 mg | | | | |
|---|---|---|---|---|---|---|---|
| | 5 mg | 10 mg | Comp A | 25 mg | Combined | | |
| | Comp A | Comp A | Fasted/Fed | Comp A | Comp A | Placebo | Overall |
| | (N = 6) | (N = 6) | (N = 7) | (N = 6) | (N = 25) | (N = 8) | (N = 33) |
| Population | n (%) | n (%) | n (%) | n (%) | n (%) | n (%) | n (%) |
| Safety | 6 (100) | 6 (100) | 7 (100) | 6 (100) | 25 (100) | 8 (100) | 33 (100) |
| PK | 6 (100) | 6 (100) | 6 (85.7) | 6 (100) | 24 (96.0) | 0 | 24 (72.7) |
| PD ECG | 6 (100) | 6 (100) | 6 (85.7) | 6 (100) | 24 (96.0) | 8 (100) | 32 (97.0) |
| PD BP | 6 (100) | 6 (100) | 7 (100) | 6 (100) | 25 (100) | 8 (100) | 33 (100) |

Abbreviations:
BP = blood pressure;
ECG = electrocardiogram;
N = total sample size;
n = subgroup sample size;
PD = pharmacodynamic;
PK = pharmacokinetic

Demographic characteristics of the safety population for Part 1 are provided in Table 2. The mean (SD) age for subjects enrolled and included in the safety population was 38.8 (10.30) years, ranging from 22 to 55 years. Nearly one-half of the subjects were white (16 of 33 subjects [48.5%]), and the remainder were black/African American (14 subjects [42.4%]) or of multiple race (3 subjects [9.1%]). The safety population was balanced with regard to sex and the mean (SD) BMI was 25.64 (3.584) kg/m².

TABLE 2
Demographic Characteristics (Part 1; Safety Population)

| | | | 15 mg | | | | |
|---|---|---|---|---|---|---|---|
| | 5 mg | 10 mg | Comp A | 25 mg | Combined | | |
| | Comp A | Comp A | Fasted/Fed | Comp A | Comp A | Placebo | Overall |
| Category | (N = 6) | (N = 6) | (N = 7) | (N = 6) | (N = 25) | (N = 8) | (N = 33) |

Age (years)

| n | 6 | | 6 | | 7 | | 6 | | 25 | | 8 | | 33 | |
|---|---|---|---|---|---|---|---|---|---|---|---|---|---|---|
| Mean (SD) | 37.8 | (9.95) | 34.3 | (11.55) | 39.3 | (9.16) | 38.8 | (11.32) | 37.6 | (10.00) | 42.6 | (10.99) | 38.8 | (10.30) |
| Median | 41.0 | | 31.0 | | 38.0 | | 36.5 | | 37.0 | | 45.0 | | 38.0 | |
| Min, max | 24, | 48 | 22, | 54 | 27, | 52 | 25, | 55 | 22, | 55 | 26, | 55 | 22, | 55 |

Weight (kg)

| n | 6 | | 6 | | 7 | | 6 | | 25 | | 8 | | 33 | |
|---|---|---|---|---|---|---|---|---|---|---|---|---|---|---|
| Mean (SD) | 74.07 | (14.961) | 71.17 | (12.206) | 71.31 | (9.585) | 76.55 | (6.614) | 73.20 | (10.715) | 83.25 | (8.949) | 75.63 | (11.080) |
| Median | 70.40 | | 74.10 | | 70.00 | | 77.20 | | 75.20 | | 81.70 | | 77.00 | |
| Min, max | 59.4, | 97.2 | 52.2, | 82.4 | 60.2, | 84.8 | 67.4, | 86.0 | 52.2, | 97.2 | 70.6, | 98.2 | 52.2, | 98.2 |

Height (cm)

| | | | | | | | |
|---|---|---|---|---|---|---|---|
| n | 6 | 6 | 7 | 6 | 25 | 8 | 33 |
| Mean (SD) | 171.17 | 172.00 | 170.14 | 168.00 | 170.32 | 173.75 | 171.15 |
| Median | 172.50 | 172.25 | 168.00 | 164.75 | 168.00 | 177.50 | 170.00 |
| Min, max | 159.0, | 161.0, | 163.0, | 163.0, | 159.0, | 158.5, | 158.5, |

BMI (kg/m²)

| n | 6 | | 6 | | 7 | | 6 | | 25 | | 8 | | 33 | |
|---|---|---|---|---|---|---|---|---|---|---|---|---|---|---|
| Mean (SD) | 24.83 | (3.802) | 23.88 | (4.297) | 24.76 | (4.306) | 26.62 | (2.367) | 25.01 | (3.695) | 27.59 | (2.486) | 25.64 | (3.584) |
| Median | 25.30 | | 22.60 | | 24.10 | | 26.90 | | 25.10 | | 28.40 | | 25.80 | |
| Min, max | 18.5, | 29.5 | 19.1, | 30.2 | 20.5, | 31.6 | 22.9, | 29.2 | 18.5, | 31.6 | 22.2, | 30.0 | 18.5, | 31.6 |

Sex, n (%)

| | | | | | | | | | | | | | | |
|---|---|---|---|---|---|---|---|---|---|---|---|---|---|---|
| Female | 3 | (50.0) | 2 | (33.3) | 4 | (57.1) | 3 | (50.0) | 12 | (48.0) | 5 | (62.5) | 17 | (51.5) |
| Male | 3 | (50.0) | 4 | (66.7) | 3 | (42.9) | 3 | (50.0) | 13 | (52.0) | 3 | (37.5) | 16 | (48.5) |

Race, n (%)

| | | | | | | | | | | | | | | |
|---|---|---|---|---|---|---|---|---|---|---|---|---|---|---|
| Black or | 2 | (33.3) | 2 | (33.3) | 4 | (57.1) | 2 | (33.3) | 10 | (40.0) | 4 | (50.0) | 14 | (42.4) |
| African | | | | | | | | | | | | | | |
| American | | | | | | | | | | | | | | |
| White | 4 | (66.7) | 2 | (33.3) | 3 | (42.9) | 4 | (66.7) | 13 | (52.0) | 3 | (37.5) | 16 | (48.5) |

| | | | | | | | | | | | |
|---|---|---|---|---|---|---|---|---|---|---|---|
| Multiple | 0 | 2 | (33.3) | 0 | 0 | 2 | (8.0) | 1 | (12.5) | 3 | (9.1) |

Ethnicity, n (%)

| Hispanic or | 1 | (16.7) | 1 | (16.7) | 0 | | 0 | | 2 | (8.0) | 0 | | 2 | (6.1) |
|---|---|---|---|---|---|---|---|---|---|---|---|---|---|---|
| Latino | | | | | | | | | | | | | | |
| Not Hispanic | 5 | (83.3) | 5 | (83.3) | 7 | (100) | 6 | (100) | 23 | (92.0) | 8 | (100) | 31 | (93.9) |
| or Latino | | | | | | | | | | | | | | |

Abbreviations:
BMI = body mass index;
max = maximum;
min = minimum;
N = total sample size;
n = subgroup sample size;
SD = standard deviation

### Part 2

Four sequential MAD cohorts were dosed. For cohorts 1, 2, and 3 of Part 2, twelve subjects were treated in each cohort of with a 7-day course of study drug (10 subjects per cohort received Compound A and 2 subjects per cohort received matching placebo) administered orally. For cohort 4 of Part 2, eleven subjects were treated with a 7-day course of study drug (10 subjects received Compound A and 1 subject received matching placebo) administered orally. The study drug (Compound A or placebo) will be provided as size 0 hard gelatin capsules. The 4 MAD dose cohorts were as follows:
Part 2, Cohort 1: 5 mg Compound A or placebo/day orally (QD), 7 days;
Part 2, Cohort 2: 10 mg Compound A or placebo/day orally (QD), 7 days;
Part 2, Cohort 3: 15 mg Compound A or placebo/day orally (QD), 7 days;
Part 2, Cohort 4: 20 mg Compound A or placebo/day orally (QD), 7 days.

Escalation to the next higher dose level in Part 2 occurred only after satisfactory review of clinical safety and pharmacokinetic data.

For each cohort of Part 2, the daily dose of study drug was administered as a single dose (1 time daily or QD). Similar to Part 1 of the study, following a screening period to determine eligibility, subjects in each cohort entered the CRU on the morning of Day −1. Study eligibility was re-confirmed upon admission to the CRU, and subjects remained in the CRU overnight prior to dosing on Day 1. Subjects received a final morning dose of study drug on the last day of dosing. Subjects were discharged from the CRU following sample collection on Day 11. Dosage administration was supervised by study personnel and all subjects completed dosing per protocol. All subjects in Cohorts 1, 2, 3, and 4 received 1 dose of study drug (Compound A or placebo) under fasting conditions once a day for 7 days. Subjects returned to the CRU on Day 23 (+2 days) for final follow-up safety assessments.

The planned 10 Compound A-treated subjects per cohort were enrolled in full and with the exception of 1 subject in cohort 2 (10 mg dose), all of the initially enrolled subjects completed the study (one subject in cohort 2 withdrew for personal reasons prior to study completion).

Table 3 shows the number of subjects in each analysis population for Part 2. Overall, of the 47 subjects randomized, all 47 were treated with at least one dose of study drug and were therefore included in the safety population. The safety population was used for all analyses of demographics and safety. The PK population included all subjects who received at least 1 dose of study drug and provided an adequate number of blood samples for the determination of at least 1 PK parameter. The PK population was used for all PK summaries and analyses.

The PD ECG population for QTcF analysis included all subjects who received at least 1 dose of study drug and for whom at least 1 pre- and post-dose QTcF result was obtained from continuous ECG extraction. The PD ECG population was used for all analyses of QTcF pharmacological effect and for correlation with PK parameters or BCX9250 concentrations. The PD BP population for analysis of changes in systolic and diastolic BP included all subjects who received at least 1 dose of study drug and for whom at least 1 pre- and post-dose BP measurement was obtained. The PD BP population was used for correlation of changes in BP with BCX9250 concentrations and/or PK parameters.

TABLE 3
Analysis Populations (Part 2)

| | 5 mg | 10 mg | 15 mg | 20 mg | Combined | | |
|---|---|---|---|---|---|---|---|
| | BCX9250 | BCX9250 | BCX9250 | BCX9250 | BCX9250 | Placebo | Overall |
| | (N = 10) | (N = 10) | (N = 10) | (N = 10) | (N = 40) | (N = 7) | (N = 47) |
| Population | n (%) | n (%) | n (%) | n (%) | n (%) | n (%) | n (%) |
| Safety | 10 (100) | 10 (100) | 10 (100) | 10 (100) | 40 (100) | 7 (100) | 47 (100) |
| PK | 10 (100) | 10 (100) | 10 (100) | 10 (100) | 40 (100) | 0 | 40 (85.1) |
| PD ECG | 10 (100) | 10 (100) | 10 (100) | 10 (100) | 40 (100) | 7 (100) | 47 (100) |
| PD BP | 10 (100) | 10 (100) | 10 (100) | 10 (100) | 40 (100) | 7 (100) | 47 (100) |

Abbreviations:
BP = blood pressure;
ECG = electrocardiogram;
N = total sample size;
n = subgroup sample size;
PD = pharmacodynamic;
PK = pharmacokinetic

Demographic characteristics of the safety population for Part 2 are provided in Table 4. The mean (SD) age for subjects enrolled and included in the safety population was 35.5 (8.46) years, ranging from 22 to 53 years. Nearly one-half of the subjects were white (23 of 47 subjects [48.9%]), the remainder being black/African American (19 subjects [40.4%]), Asian (2 subjects [4.3%]), multiple race (2 subjects [4.3%]), or American Indian/Alaska Native (1 subject [2.1%]). There were more males (25 of 47 subjects [53.2%]) than females in the safety population.

TABLE 4
Demographic Characteristics (Part 2; Safety Population)

| | | | | |
|---|---|---|---|---|
| | 5 mg | 10 mg | 15 mg | 20 mg |
| | BCX9250 | BCX9250 | BCX9250 | BCX9250 |
| Category | (N = 10) | (N = 10) | (N = 10) | (N = 10) |

Age (years)

| n | 10 | | 10 | | 10 | | 10 | |
|---|---|---|---|---|---|---|---|---|
| Mean (SD) | 40.7 | (9.01) | 35.1 | (8.79) | 32.3 | (7.15) | 35.5 | (8.02) |
| Median | 43.0 | | 35.0 | | 32.5 | | 36.5 | |
| Min, max | 26, | 53 | 23, | 48 | 23, | 49 | 22, | 49 |

Weight (kg)

| n | 10 | | 10 | | 10 | | 10 | |
|---|---|---|---|---|---|---|---|---|
| Mean (SD) | 76.20 | (12.968) | 81.84 | (13.511) | 74.32 | (16.643) | 81.73 | (14.741) |
| Median | 79.40 | | 79.80 | | 71.10 | | 83.40 | |
| Min, max | 50.2, | 91.6 | 60.2, | 105.6 | 58.5, | 114.9 | 62.6, | 107.8 |

Height (cm)

| n | 10 | | 10 | | 10 | | 10 | |
|---|---|---|---|---|---|---|---|---|
| Mean (SD) | 167.80 | (12.992) | 166.85 | (9.496) | 170.70 | (8.948) | 173.35 | (8.794) |
| Median | 165.75 | | 163.50 | | 168.50 | | 172.75 | |
| Min, max | 153.0, | 193.0 | 155.0, | 184.0 | 159.5, | 191.0 | 162.0, | 192.0 |

BMI (kg/m²)

| n | 10 | | 10 | | 10 | | 10 | |
|---|---|---|---|---|---|---|---|---|
| Mean (SD) | 26.92 | (3.393) | 29.10 | (2.703) | 25.12 | (3.854) | 26.93 | (2.917) |
| Median | 27.05 | | 29.75 | | 24.30 | | 26.80 | |
| Min, max | 20.7, | 31.4 | 22.6, | 31.8 | 20.5, | 31.5 | 21.4, | 31.5 |

Sex, n (%)

| | | | | | | | | |
|---|---|---|---|---|---|---|---|---|
| Female | 5 | (50.0) | 6 | (60.0) | 5 | (50.0) | 3 | (30.0) |
| Male | 5 | (50.0) | 4 | (40.0) | 5 | (50.0) | 7 | (70.0) |

Race, n (%)

| American Indian | 0 | | 0 | | 1 | (10.0) | 0 | |
|---|---|---|---|---|---|---|---|---|
| or Alaska Native | | | | | | | | |
| Asian | 0 | | 0 | | 0 | | 2 | (20.0) |
| Black or | 2 | (20.0) | 7 | (70.0) | 4 | (40.0) | 3 | (30.0) |
| African American | | | | | | | | |
| White | 8 | (80.0) | 3 | (30.0) | 3 | (30.0) | 5 | (50.0) |

| | | | | | |
|---|---|---|---|---|---|
| Multiple | 0 | 0 | 2 | (20.0) | 0 |

Ethnicity, n (%)

| | | | | | | | | |
|---|---|---|---|---|---|---|---|---|
| Hispanic or | 4 | (40.0) | 2 | (20.0) | 4 | (40.0) | 1 | (10.0) |
| Latino | | | | | | | | |
| Not Hispanic | 6 | (60.0) | 8 | (80.0) | 6 | (60.0) | 9 | (90.0) |
| or Latino | | | | | | | | |

| | Combined | | | | | |
|---|---|---|---|---|---|---|
| | BCX9250 | | Placebo | | Overall | |
| Category | (N = 40) | | (N = 7) | | (N = 47) | |
| Age (years) | | | | | | |
| n | 40 | | 7 | | 47 | |
| Mean (SD) | 35.9 | (8.52) | 33.1 | (8.36) | 35.5 | (8.46) |
| Median | 34.5 | | 32.0 | | 34.0 | |
| Min, max | 22, | 53 | 24, | 47 | 22, | 53 |
| Weight (kg) | | | | | | |
| n | 40 | | 7 | | 47 | |
| Mean (SD) | 78.52 | (14.366) | 76.03 | (13.480) | 78.15 | (14.124) |
| Median | 77.80 | | 81.20 | | 77.80 | |
| Min, max | 50.2, | 114.9 | 59.5, | 93.4 | 50.2, | 114.9 |
| Height (cm) | | | | | | |
| n | 40 | | 7 | | 47 | |
| Mean (SD) | 169.68 | (10.137) | 173.50 | (14.257) | 170.24 | (10.749) |
| Median | 167.00 | | 178.00 | | 167.00 | |
| Min, max | 153.0, | 193.0 | 149.5, | 190.0 | 149.5, | 193.0 |
| BMI (kg/m²) | | | | | | |
| n | 40 | | 7 | | 47 | |
| Mean (SD) | 27.02 | (3.431) | 25.13 | (4.069) | 26.74 | (3.550) |
| Median | 27.55 | | 25.60 | | 27.20 | |
| Min, max | 20.5, | 31.8 | 18.3, | 30.3 | 18.3, | 31.8 |
| Sex, n (%) | | | | | | |
| Female | 19 | (47.5) | 3 | (42.9) | 22 | (46.8) |
| Male | 21 | (52.5) | 4 | (57.1) | 25 | (53.2) |
| Race, n (%) | | | | | | |
| American Indian | 1 | (2.5) | 0 | | 1 | (2.1) |
| or Alaska Native | | | | | | |
| Asian | 2 | (5.0) | 0 | | 2 | (4.3) |
| Black or | 16 | (40.0) | 3 | (42.9) | 19 | (40.4) |
| African American | | | | | | |
| White | 19 | (47.5) | 4 | (57.1) | 23 | (48.9) |
| Multiple | 2 | (5.0) | 0 | | 2 | (4.3) |
| Ethnicity, n (%) | | | | | | |
| Hispanic or | 11 | (27.5) | 1 | (14.3) | 12 | (25.5) |
| Latino | | | | | | |
| Not Hispanic | 29 | (72.5) | 6 | (85.7) | 35 | (74.5) |
| or Latino | | | | | | |

### Assessments

Assessments made during Part 1 and Part 2 are provided in the tables below and as discussed in more detail herein.

TABLE 5
Schedule of Assessments (Part 1)

| | Screening | | Day 1 | Day 1 | | | | Follow-up |
|---|---|---|---|---|---|---|---|---|
| | Days −21 | | (pre- | (post- | | | | Visit Day 16 (+2) |
| Assessment | to −2 | Day −1 | dose) | dose) | Day 2 | Day 3 | Day 4 | (Early Termination)^{a} |
| Confinement | | X | X | X | X | X | X^{b} | |
| Physical | X | X | | X | | | | X |
| examination^{c} | | | | | | | | |
| Vital signs^{d} | X | X | X | X | X | X | X | X |
| Bedside | X | X | X | X | X | X | X | X |
| ECG^{e} | | | | | | | | |
| 12-lead | | | X | X | X | | | |
| Holter | | | | | | | | |
| monitoring^{f} | | | | | | | | |
| Pulse | | | X | X | X | X | X | |
| oximetry^{g} | | | | | | | | |
| Hematology, | X | X | | Xⁱ | X | X | X | X |
| coagulation, | | | | | | | | |
| chemistry, | | | | | | | | |
| urinalysis^{h} | | | | | | | | |
| Other | | | X | | | | X | |
| laboratory | | | | | | | | |
| assessmentsⁱ | | | | | | | | |
| Creatinine | X | X | | X | X | X | X | |
| Clearance | | | | | | | | |
| PK blood | | | X | X | X | X | X | |
| sampling^{j} | | | | | | | | |
| Urine for PK | | | X | X | X | X | X | |
| Analysis^{k} | | | | | | | | |
| Study drug | | | | X | | | | |
| administration^{l} | | | | | | | | |
| High-fat | | | X | | | | | |
| breakfast | | | | | | | | |
| (Cohort 4 | | | | | | | | |
| only)^{m} | | | | | | | | |
| Assess AEs | X | X | X | Xⁱ | X | X | X | X |
| and | | | | | | | | |
| concomitant | | | | | | | | |
| medications | | | | | | | | |

Abbreviations: AE = adverse events, BMI = body mass index; BNP = brain natriuretic peptide; CPK = creatine phosphokinase; CRU = clinical research unit; ECG = electrocardiogram; FSH = follicle-stimulating hormone; HBV = hepatitis B virus; HCV = hepatitis C virus; HIV = human immunodeficiency virus; LH = luteinizing hormone; PK = pharmacokinetic(s); PTH = parathyroid hormone; T3 = triiodothyronine; T4 = thyroxine; TSH = thyroid-stimulating hormone.
^{a}The follow-up visit was on study day 16 (+2), 12 days after discharge from the CRU. For cohort 4, the follow-up visit was 12 days after subjects are discharged from the 2^{nd} dosing period.
^{b}Subjects were discharged after completing all assessments on Day 4.
^{c}Screening and Day −1 physical examinations were full physical examinations. All post-dose physical examinations were symptom-driven assessments.
^{d}Vital signs included measurement of heart rate, blood pressure, and temperature. Heart rate, blood pressure, and temperature will be obtained pre-dose and 2, 4, 6, 8, 24, 36, 48, and 72 hours post-dose and at the follow-up visit.
^{e}Bedside 12-lead ECGS were conducted in triplicate pre-dose on Day 1; all other ECGs were allowed to be single assessments. Following dosing, ECGs will be captured at approximately 2, 4, 6, 8, 24, 48, and 72 hours post-dose. Subjects were placed on a bed in a supine position for at least 10 minutes prior to the ECGs being performed. Any blood draws scheduled at the same time occurred after obtaining the ECG.
^{f}Subjects were placed on bed rest in a supine position for at least 10 minutes prior to each Holter recording each cohort. Time points for Holter acquisition are as follows on Day 1: −1.0, −0.75, −0.5 hours prior to dosing, and 0.5, 1, 2, 3, 4, 5, 6, 8, 12, and 24 hours post-dose. The Holter acquisition period was 5 minutes. Holter leads were checked for proper connectivity prior to each time point. Time points selected for extraction and adjudication were relayed to the central cardiac laboratory and analyzed in triplicate. Any blood draws scheduled at the same time occurred after obtaining the ECG.
^{g}Pulse oximetry was to be collected pre-dose, and daily post dose with vital signs.
^{h}Hematology, clinical chemistry, coagulation, and urinalysis laboratory assessments were taken approximately 4 hours post-dose on Day 1, and then daily while subjects are in the CRU. Laboratory assessments will also be taken at the follow-up visit.
ⁱThe following laboratory tests were taken pre-dose on Day 1 and on Day 4 prior to discharge: lipids (fasting), PTH, free testosterone (taken in the morning), BNP, troponin I, 25-hydroxy vitamin D, TSH, free T3 and free T4, CPK, LH, FSH, and estradiol.
^{j}Plasma for Compound A PK analysis was collected pre-dose on Day 1 and at 0.5, 1, 2, 3, 4, 5, 6, 8, 10, 12, 16, 24, 48, and 72 hours post-dose.
^{k}For the analysis of urinary excretion of Compound A, an aliquot of urine was collected in all fasting cohorts pre-dose (0 hour), and all urine was collected for the following post-dose intervals: 0-6 hours, 6-12 hours, 12-24 hours, 24-48 hours and 48-72 hours. No urine samples were collected after the fed dose is administered in Cohort 4.
^{l}All doses were administered after an overnight fast of at least 8 hours with the exception of the second dose administered in Part 1 Cohort 4.
^{m}Cohort 4 received 2 doses of Compound A separated by at least 7 days or 5 half-lives of BCX9250 (whichever was longer). The first dosing period received the blinded drug fasted. Prior to the second dose in Part 1 Cohort 4, a high-fat breakfast was served 30 minutes prior to dosing the blinded study drug. Study subjects were expected to consume at least 90% of the meal within ≤25 minutes, and study drug was been administered 30 minutes after the start of the meal. The follow up visit was after the 2nd dosing period.

TABLE 5
Schedule of Assessments (Part 2)

| | Screening | | Day 1 | Day 1 | | | | | | Follow-up |
|---|---|---|---|---|---|---|---|---|---|---|
| | Days −21 | Day | (pre- | (post- | Days | Day | Days | Day | Day | Visit Day 23 (+2) |
| Assessment | to −2 | −1 | dose) | dose) | 2-6 | 7 | 8-10 | 11 | 14^{a} | (Early Termination)^{b} |
| Confinement | | X | X | X | X | X | X | X^{c} | | |
| Physical | X | X | | X | X | X | | X | | X |
| examination^{d} | | | | | | | | | | |
| Vital signs^{e} | X | X | X | X | X | X | X | X | | X |
| Pulse | | | X | X | X | X | X | X | | |
| Oximetry^{f} | | | | | | | | | | |
| Bedside | X | X | X | X | X | X | X | X | | X |
| ECG^{g} | | | | | | | | | | |
| 12-lead | | | X | X | | X | | | | |
| Holter | | | | | | | | | | |
| monitoring^{h} | | | | | | | | | | |
| Hematology, | X | X | | X^{h} | X^{h} | X^{h} | | X | | X |
| coagulation, | | | | | | | | | | |
| chemistry, | | | | | | | | | | |
| urinalysisⁱ | | | | | | | | | | |
| Other | | | X | | | | | X | | |
| Laboratory | | | | | | | | | | |
| Assessments^{j} | | | | | | | | | | |
| Creatinine | X | | | X | X | X | | X | | |
| Clearance | | | | | | | | | | |
| PK blood | | | X | X | X | X | X | | X | |
| sampling^{k} | | | | | | | | | | |
| Urine for PK | | | | X | X | X | X | | | |
| Analysis^{l} | | | | | | | | | | |
| Study drug | | | | X | | | | | | |
| administration^{m} | | | | | | | | | | |
| Assess AEs | X | X | X | X | X | X | X | X | | |
| and | | | | | | | | | | |
| concomitant | | | | | | | | | | |
| medications | | | | | | | | | | |

Abbreviations: AE = adverse events, BMI = body mass index; BNP = brain natriuretic peptide; BSAP = bone-specific alkaline phosphatase; CPK = creatine phosphokinase; CRU = clinical research unit; CTX = serum C-telopeptide crosslink ECG = electrocardiogram; FSH = follicle-stimulating hormone; h = hours; HBV = hepatitis B virus; HCV = hepatitis C virus; HIV = human immunodeficiency virus; LH = luteinizing hormone; PINP = amino-terminal propeptide of Type I procollagen; PK = pharmacokinetics; PTH = parathyroid hormone; T3 = triiodothyronine; T4 = thyroxine; TSH = thyroid-stimulating hormone.
^{a}If needed based on Compound A half-life, subjects will return to the clinic on Day 14 (+1) for a PK sample collection
^{b}The follow-up visit was on study day 23 (+2), 12 days after discharge from the CRU.
^{c}Subjects were discharged after completing all assessments on Day 11.
^{d}Screening and Day −1 physical examinations were full physical examinations. All post-dose physical examinations were symptom-driven assessments.
^{e}Vital signs included measurement of heart rate, blood pressure, and temperature. Heart rate, blood pressure, and temperature will be obtained pre-dose and 2, 4, 6, 8, 24, 36, 48, and 72 hours post-dose on Day 1 and 7, and daily on Days 4-6, and at the follow-up visit.
^{f}Pulse oximetry was measured pre-dose on Day 1 and daily with vital signs.
^{g}Bedside 12-lead ECGS were conducted in triplicate pre-dose on Day 1; all other ECGs were allowed to be single assessments. Following dosing, ECGs were captured at approximately 2, 4, 6, 8, 24, 48, and 72 hours post-dose on Day 1 and 7. Subjects were placed on a bed in a supine position for at least 10 minutes prior to the ECGs being performed. Any blood draws scheduled at the same time occurred after obtaining the ECG.
^{h}Subjects were placed on bed rest in a supine position for at least 10 minutes prior to each Holter recording each cohort. Time points for Holter acquisition are as follows on Day 1 and Day 7: −1.0, −0.75, −0.5 hours prior to dosing, and 0.5, 1, 2, 3, 4, 5, 6, 8, 12, and 24 hours post-dose. The Holter acquisition period was 5 minutes. Holter leads were checked for proper connectivity prior to each time point. Time points selected for extraction and adjudication were relayed to the central cardiac laboratory and analyzed in triplicate. Any blood draws scheduled at the same time occurred after obtaining the ECG.
ⁱHematology, clinical chemistry, coagulation, and urinalysis laboratory assessments were taken approximately 4 hours post-dose each day of dosing.
^{j}The following laboratory tests were to be taken pre-dose on Day 1 and on Day 11 prior to discharge: lipids (fasting), free testosterone (taken in the morning), 25-hydroxy vitamin D, BNP, troponin I, PTH, TSH, free T3 and free T4, CPK, LH, FSH, estradiol, CTX, BSAP, and PINP.
^{k}Plasma for Compound A PK analysis was collected pre-dose on Day 1 and Day 7 and at 0.5, 1, 2, 3, 4, 5, 6, 8, 10, 12, 16, 24, 48, and 72 hours post-dose on both days.
^{l}For the analysis of urinary excretion of Compound A, an aliquot of urine was collected in fasting cohorts pre-dose (0 hour) and all urine was collected for the following post-dose intervals on Day 1 and Day 7: 0 to 6, 6 to 12, and 12 to 24 hours. Daily 24 hour intervals were collected on Days 2 through 6.
^{m}All doses were administered after an overnight fast of at least 8 hours.

Serial venous blood samples were withdrawn via an indwelling cannula or by venipuncture according to the schedule in Tables 4 and 5 for Parts 1 and 2, respectively. Samples were collected into 4 mL lithium heparin tubes. The acceptable deviations from the nominal blood sampling times in Part 1 and Part 2 are as follows: the pre-dose blood sample will be taken ≤1 hour before dosing; 0 to 1 hour post-dose samples will be taken within +2 minutes of the nominal post-dose sampling time; >1 to 12 hours post-dose samples will be taken within +10 minutes of the nominal post-dose sampling time; and >12 hours post-dose samples will be taken within +30 minutes of the nominal post-dose sampling time if subjects are resident in the clinic.

All plasma samples for the determination of Compound A concentrations will be analyzed using a validated liquid chromatography-mass spectroscopy assay. The PK parameters that will be estimated are summarized in Table 6 below. Additional analyses may be conducted as appropriate to determine additional parameters as needed.

TABLE 6

| Pharmacokinetic | |
|---|---|
| Parameter | Definition |
| AUC | area under the concentration versus time curve |
| AUC_{0-inf} (Part I only) | AUC extrapolated to infinite time, calculated as AUClast + (Clast/λz) |
| AUC₀₋ₜ | AUC from time zero to time “t” |
| % AUCₑₓₜᵣₐₚ (Part I only) | Percentage of AUC extrapolated between AUClast and AUC0-inf |
| AUCₜₐᵤ (Part 2 only) | AUC over the dosing interval (tau) (i.e., 24 hours for once daily dosing) |
| C_{av,ss} (Part 2 only) | Average steady-state plasma drug concentration during multiple-dose |
| | administration |
| Cₗₐₛₜ | last measurable concentration of drug |
| Cₘₐₓ | maximum observed concentration of drug |
| Cₜₐᵤ (Part 2 only) | observed drug concentration at the end of the dosing interval (tau) |
| R_{ac} (Part 2 only) | accumulation ratio |
| t^{1/2} | estimate of the terminal elimination half-life of the drug |
| CL/F | apparent oral clearance after administration of the drug: CL/F = Dose/AUC where |
| | “Dose” is the dose of the drug and AUC = AUC0-inf after a single dose and AUCtau |
| | at steady state |
| λ_{z} | terminal elimination rate constant, estimate by linear regression of the terminal |
| | elimination phase of the concentration of drug versus time curve |
| V_{z}/F | apparent volume of distribution of the drug |
| Tₘₐₓ | time of Cmax |
| CLᵣ | renal clearance of unchanged drug in a specific interval (CLr[interval]) or |
| | cumulatively over |
| | all collection intervals |
| % excreted | percentage of given dose excreted in the urine as unchanged drug |

In all derivations of PK parameters, 0 will be substituted for concentrations below the quantification limit (BQL) of the assay. Samples that are BQL but are between 2 samples with detectable concentrations will be excluded from PK analysis.

Urine samples were collected according to the schedule in Tables 4 and 5 for Parts 1 and 2, respectively. The pre-dose sample on Day 1 was collected any time in the 2 hours prior to dosing. A 30-minute window was allowed around collection intervals. For subjects randomized to placebo, no urine was analyzed for Compound A concentrations.

Separately for Parts 1 and 2, dose proportionality was assessed by comparing PK parameters of Compound A across evaluated dose levels. Dose proportionality was evaluated over all fasted doses within a study part based upon AUC_{0-inf} (Part 1), AUCₜₐᵤ (Part 2), AUC₀₋ₜ, and Cₘₐₓ using the power model. The analysis was performed for Day 1 (Parts 1 and 2) and Day 7 (Part 2).

Dose proportionality for PK parameters was assessed by restricted maximum likelihood using SAS PROC MIXED (SAS STAT software, Cary N.C.). The mean slope was estimated from the power model and the corresponding 90% confidence interval (CI) calculated. Dose proportionality was also examined using an analysis of variance (ANOVA) method, where PK parameters for each dose were compared with a reference dose on a pair-wise basis. The ratio of geometric least squares (GLS) means and the corresponding 90% CI was estimated for each PK parameter of interest.

Accumulation ratios were calculated using AUC values from serial sampling after the first and last dose in Part 2. Accumulation for each Compound A dose evaluated in Part 2 was evaluated by comparing Day 7 AUCₜₐᵤ to Day 1 AUC₀₋ₜ (with t being the length of the dosing regimen). A mixed-effects model with a fixed effect for day and a random subject effect was used to estimate the difference between AUCₜₐᵤ and AUC₀₋ₜ on the log scale.

To assess the effect of food (high-fat meal) on the PK of Compound A in Cohort 4 of Part 1, log-transformed PK parameters (AUC_{0-inf}, AUC₀₋ₜ, and Cₘₐₓ) were analyzed by ANOVA. Mixed-effects models were constructed with treatment fitted as a fixed effect and subject fitted as a random effect to provide point estimates for the ratios of PK parameters under fed relative to fasted conditions and their associated 90% CI. A 90% CI around the GLS mean ratio of AUC_{0-inf}; AUC₀₋ₜ, and Cₘₐₓ was calculated for each comparison (i.e., fed versus fasted) for the effect of a high-fat meal on the bioavailability of Compound A.

Time to steady-state was evaluated in Part 2 using C_{trough} (observed plasma concentration immediately before dosing) levels, where sufficient samples existed to determine steady state. The following timepoints were used in the analysis for once daily dosing: 24, 48, and 72 hours post dose (for Day 1) and pre-dose and 24 hours post dose on the last day of dosing (Day 7).

ECGs were obtained according to the schedule in Tables 4 and 5 for Parts 1 and 2, respectively. A standard bedside 12-lead ECG machine or telemetry system that calculates HR and measures the PR, QRS, QT, RR, and QTc (QTcF) intervals was used. The acceptable deviations from the nominal ECG measurement time points were: re-dose: ≤2 hours before dosing; post dose: ±15 minutes from the nominal post-dose time point; and discharge: ±1 hour from the nominal time point.

Where possible, Holter-obtained and adjudicated ECGs were used to determine the change from baseline in mean differences in QTcF between each active treatment chosen to be analyzed relative to that obtained on subjects receiving placebo within a study part (for Part 2, within each cohort). At each time point Holter ECGs are analyzed, an individual subject's change from baseline will be calculated as:

Δᵢₖ=(QTcF for subject I at time point k−Baseline QTcF), where QTcF measurements will be the average of triplicate ECGs at each time point. Baseline QTcF is the average of all pre-dose values.

Where possible, systolic and diastolic BP measurements will be used to determine the change from baseline between each active treatment relative to that obtained on subjects receiving placebo within a study part. At each time point blood pressure data is collected, an individual subject's change from baseline will be calculated.

Exposure-response analyses of the relationships between QTcF changes and Compound A plasma concentrations will be explored using model-based techniques. Exposure-response analyses of the relationships between BP changes and Compound A plasma concentrations will be explored using model-based techniques.

### Pharmacokinetic Results

### Part 1

Thirty-two healthy subjects completed dosing in the 4 SAD cohorts (5 mg, 10 mg, 15 mg, and 25 mg) in Part 1. For all cohorts of Part 1, 8 subjects were enrolled per Cohort (6 Compound A and 2 placebo treated subjects per cohort). One subject discontinued from the study (Cohort 4) after completing first dose (fasted) and was replaced for the second dose (fed). Subjects in all cohorts of Part 1 were orally administered study drug (Compound A or placebo) in a single dose administered as a single capsule. For cohort 4, period 1, subjects initially received a first single dose of study drug (Compound A or placebo) as described above under fasting conditions (fasted). For cohort 4, period 2, after a washout period as described herein all subjects were given a second single dose of study drug (Compound A or placebo) as described above after a high-fat meal (fed). Subjects in cohort 4 received the same study drug and dose (Compound A or placebo) in both periods 1 and 2.

Compound A administered as ascending oral doses of 5 mg to 25 mg was safe and generally well tolerated in all subjects in all Cohorts. There were no study discontinuations due to adverse events, no grade 3 or 4 adverse events, and no clinically significant changes in vital signs, electrocardiograms, or safety laboratory parameters. No safety signals were observed.

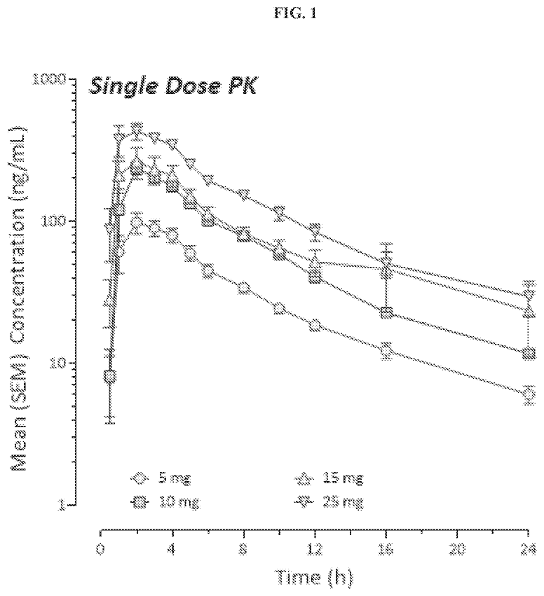

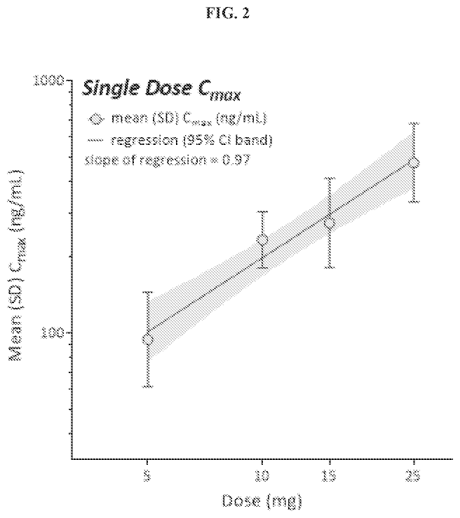

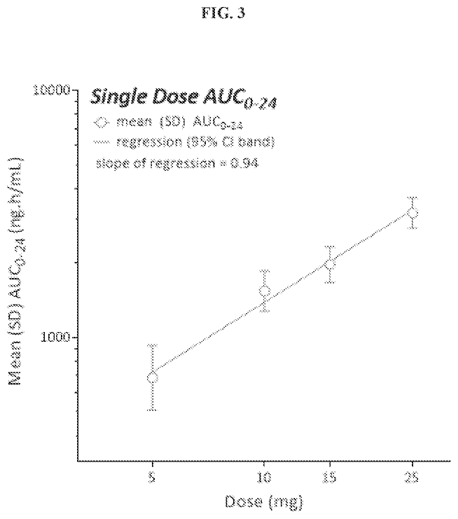

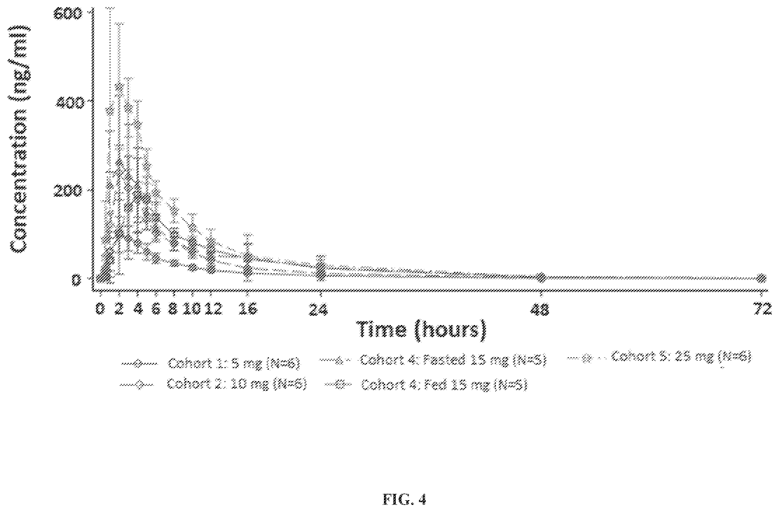

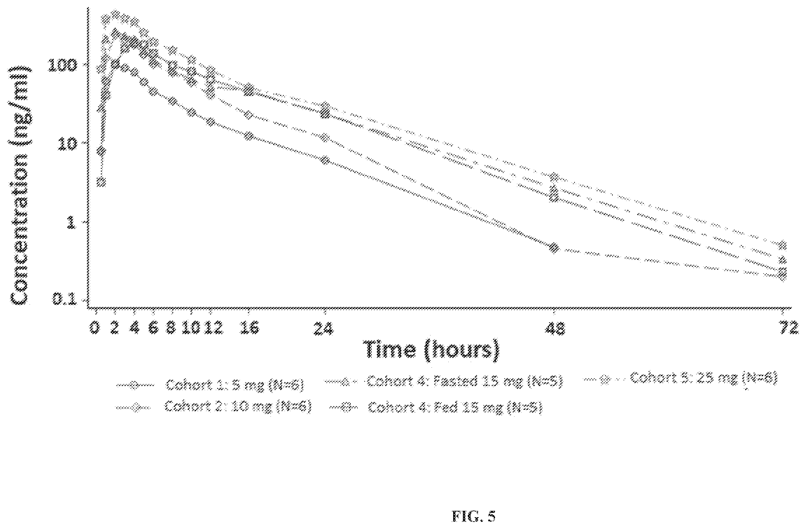

PK parameters of ascending oral doses of Compound A from 5 mg to 25 mg for all cohorts in Part 1 were well characterized over the 24-hour sampling period (FIG. 1). Furthermore, administration of Compound A was approximately linear and dose proportional (FIGS. 2 and 3) for the 5 to 25 mg dose range of Part 1. The mean plasma concentration vs. time profiles after oral administration of SADs of Compound A from 5 mg to 25 mg are shown in linear and semi-log plots in FIGS. 4 and 5, respectively. For all cohorts except cohort 1 (5 mg dose), Compound A remained detectable over the entire 72-hour data collection period.

Plasma PK parameters after oral administration of SAD of Compound A from 5 mg to 25 mg are presented in Table 7. For cohort 4 (15 mg dose), parameters are presented for both fasted and fed conditions. Dosing for all other cohorts was conducted under fasted conditions. For all of the fasted cohorts, median time to maximum concentration (Tₘₐₓ) was consistently achieved at approximately 2 hours after dosing. The median t_{1/2} ranged from 5.8 to 7.6 hours over all of the cohorts, with no apparent relationship to dose.

TABLE 7
Summary of Compound A Plasma Pharmacokinetic Parameters Following
Single Ascending Doses (PK Population)

| Pharmacokinetic | 5 mg | 10 mg | 15 mg fasted | 15 mg fed | 25 mg |
|---|---|---|---|---|---|
| Parameter | (N = 6) | (N = 6) | (N = 5) | (N = 5) | (N = 6) |
| Cₘₐₓ (ng/mL) ^{a} | 94.24 (45.2) | 234.3 (26.1) | 272.8 (42.5) | 199.5 (41.2) | 474.1 (37.1) |
| Tₘₐₓ (h) ^{b} | 2.011 | 2.013 | 2.009 | 4.023 | 2.071 |
| | (1.01, 3.01) | (1.14, 2.02) | (2.01, 16.0) | (3.00, 12.0) | (1.00, 3.01) |
| AUC₀₋₂₄ (ng · h/mL) ^{a} | 686.9 (30.7) | 1545 (18.9) | 1976 (16.6) | 1742 (17.6) | 3188 (14.2) |
| AUC₀₋ₗₐₛₜ (ng · h/mL) ^{a} | 717.0 (31.3) | 1579 (18.8) | 2178 (24.8) | 1898 (24.3) | 3475 (17.8) |
| AUC_{0-inf} (ng · h/mL) ^{a} | 755.7 (29.9) | 1662 (17.3) | 2214 (24.3) | 1978 (21.4) | 3517 (17.0) |
| t_{1/2} (h) ^{b} | 6.833 | 5.753 | 7.639 | 6.688 | 7.502 |
| | (6.39, 9.94) | (4.63, 13.0) | (4.69, 9.26) | (4.62, 8.54) | (4.09, 9.43) |
| CL/F (L/h) ^{a} | 7.279 (30.7) | 6.474 (18.9) | 7.593 (16.6) | 8.611 (17.6) | 7.843 (14.2) |
| V_{z}/F (L) ª | 77.33 (36.3) | 59.94 (51.4) | 74.85 (21.8) | 81.54 (33.2) | 75.84 (34.1) |

Abbreviations:
AUC = area under the concentration-time curve;
AUC₀₋₂₄ = AUC from time 0 to 24 hours;
AUC_{0-inf} = AUC from time 0 extrapolated to infinite time;
AUC₀₋ₗₐₛₜ = AUC from time 0 to the last measurable concentration;
Cₘₐₓ = maximum concentration;
CL/F = apparent oral clearance after administration of the drug;
CV = coefficient of variation;
max = maximum;
min = minimum;
N = sample size;
PK = pharmacokinetic;
t_{1/2} = half-life;
Tₘₐₓ = time to Cₘₐₓ;
V_{z}/F = apparent volume of distribution of the drug
^{a} Data reported as geometric mean (CV % of geometric mean).
^{b} Data reported as median (min, max).

An evaluation of the effect of food on Compound A PK was conducted in SAD cohort 4. Subjects received a single dose of 15 mg Compound A in the fasted state and following a high-fat meal, separated by at least 7 days. Compound A levels after a high fat meal (fed) were similar to those after dosing on an empty stomach (fasted). After a high-fat meal, the GLSM Cₘₐₓ and AUC₀₋₂₄ values appeared to be modestly decreased (27% and 12% as compared to the fasted values, respectively) whereas the GLSM AUC₀₋ₗₐₛₜ and GLSM AUC_{0-inf} fasted vs. fed values were approximately equivalent. For Tₘₐₓ, the median of paired differences (95% CI) for the fed vs. fasted subjects was 2.00 (0.99, 10.00) with a Wilcoxon signed-rank test statistic (P value) of 5.00 (0.1250).

TABLE 8
Statistical Comparisons of Compound A PK
Parameters Following Single Dose Administration at
15 mg under Fasted or Fed Conditions (PK Population)

| | Geometric least- | | % Geometric |
|---|---|---|---|
| | squares means ª | | least square |
| Pharmacokinetic | 15 mg fed | 15 mg fasted | mean ratio (90% CI) |
| Parameter | (N = 5) | (N = 5) | 15 mg fed/fasted |
| Cₘₐₓ (ng/ml) | 199.50 | 272.80 | 73.13 (45.59, 117.32) |
| AUC₀₋ₗₐₛₜ (ng · h/mL) | 2015.24 | 2019.55 | 99.79 (85.20, 116.88) |
| AUC₀₋₂₄ (ng · h/mL) | 1742.05 | 1975,63 | 88.18 (72.21, 107.68) |
| AUC_{0-inf} (ng · h/mL) | 2087.05 | 2075.34 | 100.56 (84.37, 119.87) |

Abbreviations:
AUC = area under the concentration-time curve;
AUC₀₋₂₄ = AUC from time 0 to 24 hours;
AUC_{0-inf} = AUC from time 0 extrapolated to infinite time;
AUC₀₋ₗₐₛₜ = AUC from time 0 to the last measurable concentration;
CI = confidence interval;
Cₘₐₓ = maximum concentration;
N = sample size;
PK = pharmacokinetic
ª Geometric least-squares means were obtained using a mixed-effects model utilizing the ln-transformed values of plasma BCX9250 for AUC₀₋ₗₐₛₜ, AUC₀₋ₜ, AUC_{0-inf}, and Cₘₐₓ parameters for the PK population.

Compound A urinary PK parameters following administration of SAD of Compound A from 5 mg to 25 mg are summarized in Table 9. For single doses ranging from 5 to 25 mg, the cumulative amount of parent compound excreted in the urine was less than 0.3% of the administered dose in all cohorts, and no apparent dose relationship was identified.

TABLE 9
Summary of Compound A Urinary PK Parameters
Following Single Ascending Doses (PK Population)

| | | | 15 mg | 25 mg |
|---|---|---|---|---|
| Pharmacokinetic | 5 mg | 10 mg | fasted | BCX9250 |
| Parameter ª | (N = 6) | (N = 6) | (N = 5) | (N = 6) |
| CumAe₀₋₇₂ (ng) | 13,340 (43.6) | 12,060 (84.0) | 21,130 (79.5) | 48,210 (87.0) |
| Cum % | 0.2668 (43.6) | 0.1206 (84.0) | 0.1409 (79.5) | 0.1928 (87.0) |
| excreted₀₋₇₂ | | | | |
| (%) | | | | |
| CL_{R,0-6} (mL/h) | 23.63 (28.3) | 9.499 (79.6) | 12.84 (56.2) | 15.98 (99.4) |
| CL_{R,6-12} (mL/h) | 14.61 (92.8) | 6.137 (93.2) | 6.793 (98.8) | 10.56 (138.2) |
| CL_{R,12-24} (mL/h) | 8.576 (263,3) | 4.759 (56,5) | 8.008 (114.1) | 9.785 (145.0) |
| CL_{R,24-48} (mL/h) | N/A ^{b} | N/A ^{b} | 7.674 (80.2) | 10.44 (87.9) |

Abbreviations:
CL_{R,t1-t2} = renal clearance during the time interval specified by t1 and t2;
CumAe₀₋₇₂ = cumulative amount of BCX9250 excreted in the urine from time 0 to 72 hours;
Cum % excreted₀₋₇₂ = CumAe₀₋₇₂ expressed as a percentage of the total dose;
CV = coefficient of variation;
N = sample size;
N/A = not applicable;
PK = pharmacokinetic
ª All data reported as geometric mean (CV % of geometric mean).
^{b} Geometric mean (CV % of geometric mean) were not calculated as renal clearance was 0 for all subjects over this interval.

The results from Part 1 show oral dosing with Compound A was safe and generally well tolerated following single doses of 5, 10, 15, and 25 mg. Safety assessments including TEAEs, clinical laboratory evaluations, vital signs, and bedside 12-lead ECG evaluations were overall unremarkable. There was no dose- or duration-related increase in frequency or severity in clinical laboratory or vital sign assessments. Oral dosing with Compound A demonstrated linear and dose-proportional exposure.

### Part 2

Forty-six healthy subjects completed dosing in the 4 MAD cohorts (5 mg, 10 mg, 15 mg, and 20 mg) in Part 2. For Cohorts 1-3 of Part 2, 12 subjects were enrolled per Cohort (10 Compound A and 2 placebo treated subjects per cohort). For Cohort 4 of Part 2, 11 subjects were enrolled (10 Compound A and 1 placebo treated subject; the last placebo treated subject was not enrolled due to impact of COVID-19 on screening) Subjects in Cohorts 1-4 of Part 2 were orally administered (Compound A or placebo) as described above under fasting conditions.

Compound A administered as multiple ascending oral doses of 5 mg to 20 mg over 7 days was safe and generally well tolerated in all subjects in all cohorts There were no study discontinuations due to adverse events, no grade 3 or 4 adverse events, and no clinically significant changes in vital signs, electrocardiograms, or safety laboratory parameters. No safety signals were observed.

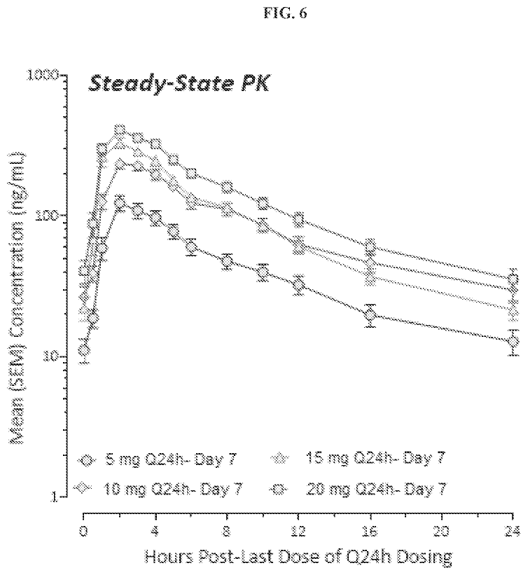

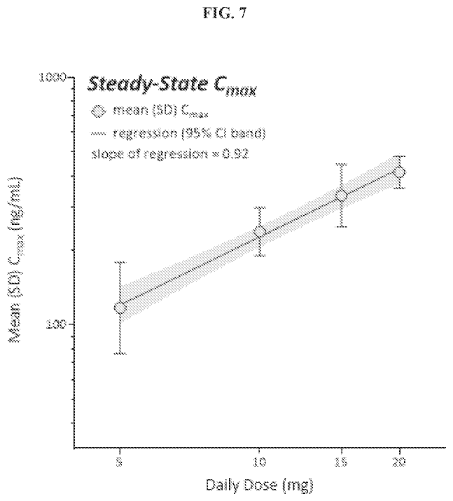

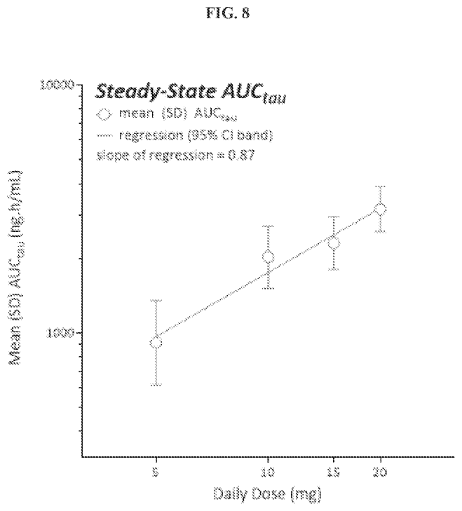

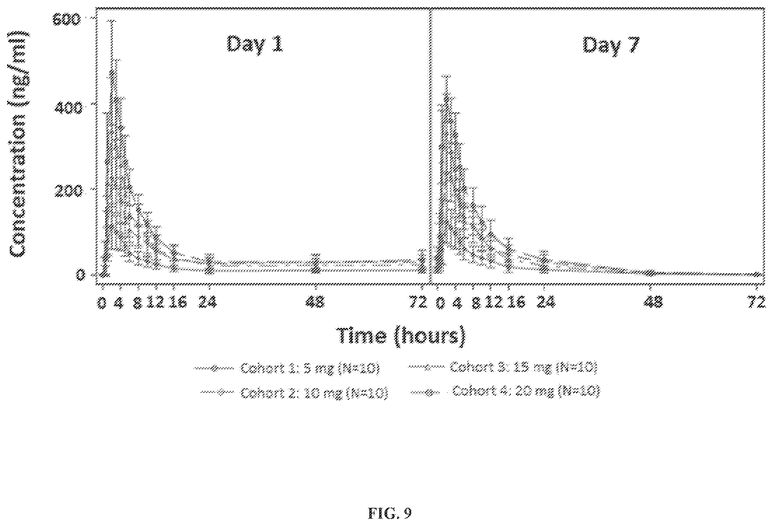

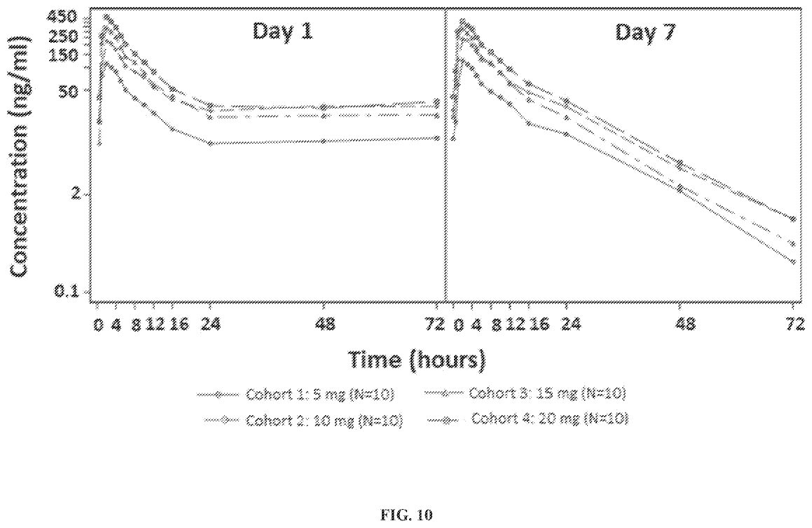

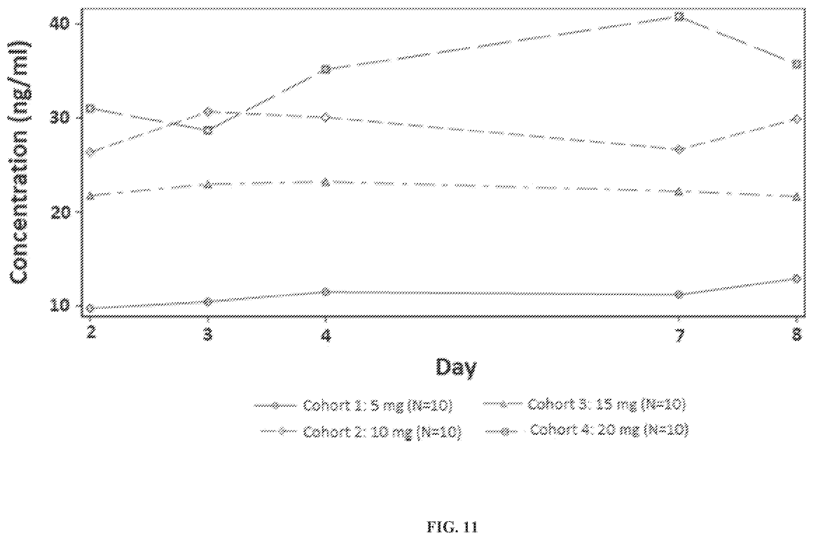

PK parameters of multiple ascending oral doses of Compound A from 5 mg to 20 mg for all Cohorts in Part 2 were well characterized over the sampling period (FIG. 6). Furthermore, administration of Compound A was approximately linear and dose proportional (FIGS. 7 and 8) for the 5 to 20 mg MAD dose range of Part 2. Drug exposure (area under the curve) at a dose of 20 mg QD in Cohort 4 after 7 days administration was similar to that achieved with doses that suppressed HO in a nonclinical model of activity of orally dosed Compound A. The mean plasma concentration vs. time profiles after oral administration of the first dose of Compound A on Day 1 and after the last dose of Compound A on Day 7 at 5, 10, 15, and 20 mg QD are shown on linear and semi-log plots in FIGS. 9 and 10, respectively. Trough concentrations following once-daily doses on Day 2 and Day 3 are shown at the 48- and 72-hour timepoints in the Day 1 plots. In addition, a plot of mean trough plasma levels of Compound A concentrations over time is shown in FIG. 11. Trough concentrations were similar at and beyond the 48-hour timepoint across cohorts, suggesting that steady state was achieved by Day 3 of dosing.

Plasma PK parameters after oral administration of the first dose of Compound A on Day 1 and after the last dose of Compound A on Day 7 at 5, 10, 15, and 20 mg QD doses are presented in Tables 10 and 11, respectively. On both Days 1 and 7, a median Tₘₐₓ of approximately 2.0 hours was observed consistently across all of the dosing cohorts. After the last dose on Day 7, the median terminal elimination half-lives were generally consistent across cohorts and ranged from 7.5 to 8.9 hours.

TABLE 10
Summary of Compound A Plasma PK Parameters Following Multiple
Ascending Doses (Day 1; PK Population)

| Pharmacokinetic | 5 mg | 10 mg | 15 mg | 20 mg |
|---|---|---|---|---|
| Parameter | (N = 10) | (N = 10) | (N = 10) | (N = 10) |
| Cₘₐₓ (ng/ml) ^{a} | 105.0 (45.9) | 217.0 (46.2) | 324.2 (34.9) | 464.1 (26.1) |
| Tₘₐₓ (h) ^{b} | 2.009 (1.01, 4.01) | 2.009 (2.01, 3.01) | 2.006 (1.01, 4.00) | 2.007 (2.00, 3.00) |
| AUC₀₋₂₄ (ng · h/mL) ^{a} | 797.9 (37.8) | 1703 (37.4) | 2204 (39.0) | 3160 (21.2) |

Abbreviations:
AUC₀₋₂₄ = area under the concentration-time curve from time 0 to 24 hours;
Cₘₐₓ = maximum concentration;
CV = coefficient of variation;
max = maximum;
min = minimum;
N = sample size;
PK = pharmacokinetic;
Tₘₐₓ = time to Cₘₐₓ
^{a} Data reported as geometric mean (CV % of geometric mean).
^{b} Data reported as median (min, max).

TABLE 11
Summary of Compound A Plasma PK Parameters Following 7 Days of
Multiple Oral Doses (Day 7; PK Population)

| Pharmacokinetic | 5 mg | 10 mg | 15 mg | 20 mg |
|---|---|---|---|---|
| Parameter | (N = 10) | (N = 10) | (N = 10) | (N = 10) |
| Cₘₐₓ (ng/ml) ª | 117.0 (44.4) | 238.0 (22.6) | 333.5 (29.7) | 413.9 (14.9) |
| Tₘₐₓ (h) ^{b} | 2.007 (1.01, 3.03) | 2.021 (2.01, 3.01) | 2.006 (1,01, 3.00) | 2.001 (1.00, 3.00) |
| AUCₜₐᵤ (ng · h/mL) ^{a} | 915.5 (40.7) | 2023 (29.7) | 2303 (24.9) | 3185 (24.4) |
| t_{1/2} (h) ^{b} | 8,846 (4.73, 25.1) | 8.512 (6.09, 12.3) | 7.534 (4.05, 12.2) | 8.332 (4.61, 11.0) |
| Cₜₐᵤ (ng/ml) ^{a} | 10.70 (72.6) | 25.47 (68.1) | 19.02 (59.6) | 30.75 (66.5) |
| CL/F (L/h) ^{a} | 5.462 (40.7) | 4.943 (29.7) | 6.512 (24.9) | 6.280 (24.4) |
| V_{z}/F (L) ª | 70.49 (63.5) | 62.41 (13.1) | 69.82 (32.5) | 75.35 (20.2) |
| Accumulation ratio ^{a,c} | 1.147 (15.0) | 1.188 (27.3) | 1.045 (28.0) | 1.008 (17.0) |

Abbreviations:
AUCₜₐᵤ = area under the concentration-time curve over the dosing interval;
Cₘₐₓ = maximum concentration;
Cₜₐᵤ = observed drug concentration at the end of the dosing interval;
CL/F = apparent oral clearance after administration of the drug;
CV = coefficient of variation;
max = maximum;
min = minimum;
N = sample size;
PK = pharmacokinetic;
t_{1/2} = half-life;
Tₘₐₓ = time to Cmax;
V_{z}/F = apparent volume of distribution of the drug
^{a} Data reported as geometric mean (CV % of geometric mean).
^{b} Data reported as median (min, max).
^{c} Calculated as AUCₜₐᵤ (Day 7)/AUC₀₋₂₄ (Day 1).

The GLSM Day 1 AUC₀₋₂₄ and Day 7 AUCₜₐᵤ for all of the MAD dosing cohorts and the corresponding percent accumulation ratios for Part 2 are shown in Table 12. No accumulation or minimal accumulation was observed for each MAD cohort, with accumulation ratios (90% CI) equal to 114.74 (101.88 to 129.22) for cohort 1 (5 mg QD), 118.76 (105.45, 133.75) for cohort 2 (10 mg QD), 104.48 (92.77, 117.67) for cohort 3 (15 mg QD), and 100.80 (89.50, 113.51) for cohort 4 (20 mg QD). The 90% CI for the % accumulation ratio overlapped for all cohorts, suggesting that the mild accumulation observed for cohorts 1 and 2 may be due to random variability rather than an actual PK mechanism. Overall, minimal accumulation over 7 days of QD dosing was consistent with the comparatively short terminal elimination t_{1/2} for Compound A.

TABLE 12
Statistical Analysis of Plasma Accumulation Ratios of Compound A
Following Multiple Dose Administration (PK Population)

| | Geometric Least Square Means ^{a} | | |
|---|---|---|---|
| | Day 1 | Day 7 | |
| | AUC₀₋₂₄ | AUCₜₐᵤ | |
| Treatment | (ng · h/mL) | (ng · h/mL) | % Accumulation |
| Group | (N = 10) | (N = 10) | Ratio (90% CI) |
| 5 mg QD | 797.89 | 915.48 | 114.74 (101.88, 129.22) |
| 10 mg QD | 1703.36 | 2022.93 | 118.76 (105.45, 133.75) |
| 15 mg QD | 2204.47 | 2303.26 | 104.48 (92.77, 117.67) |
| 20 mg QD | 3159.74 | 3184.88 | 100.80 (89.50, 113.51) |

Abbreviations:
AUC₀₋₂₄ = area under the concentration-time curve from time 0 to 24 hours;
AUCₜₐᵤ = area under the concentration-time curve over the dosing interval;
CI = confidence interval;
QD = once-daily dosing
ª Computed using a mixed-effects model with natural logarithm (ln)-transformed values of AUC₀₋₂₄ and AUCₜₐᵤ as the dependent variable and terms for treatment, visit, and treatment-by-visit interaction as fixed independent variables and subject as a random effect.

Compound A urinary PK parameters following administration of MAD of 5, 10, 15 and 20 mg are summarized in Table 13. Similar to urinary excretion observed in the SAD cohorts of Part 1, the cumulative amount of parent compound excreted in the urine was less than 1% for MAD dose levels ranging from 5 to 20 mg QD, and no apparent dose relationship was identified.

TABLE 13
Summary of Compound A Urinary PK Parameters Following Multiple
Ascending Doses (PK Population)

| | 5 mg | 10 mg | 15 mg | 20 mg |
|---|---|---|---|---|
| PK Parameter ª | (N = 10) | (N = 10) | (N = 10) | (N = 10) |
| CumAe₀₋₁₆₈ (ng) | 88,030 (114.5) | 84,600 (66.5) | 176,000 (89,5) | 238,600 (47.2) |
| Cum % excreted₀₋₁₆₈ (%) | 0.2515 (114.5) | 0.1209 (66.5) | 0.1676 (89.5) | 0.1705 (47.2) |
| CL_{R,0-6} (mL/h) | 17.16 (90.4) | 9.850 (75.1) | 14.19 (131.3) | 13.36 (76.2) |
| CL_{R,6-12} (mL/h) | 11.11 (152.2) | 5.468 (73.0) | 7.172 (100.3) | 9.673 (64.5) |
| CL_{R,32-24} (mL/h) | 12.32 (83.5) | 5.694 (65.7) | 6.837 (92.5) | 7.595 (39.1) |
| CL_{R,144-150} (mL/h) | 22.90 (79.5) | 12.01 (64.4) | 11.49 (171.8) | 15.28 (85.8) |
| CL_{R,150-156} (mL/h) | 10.85 (144.3) | 4.787 (168.5) | 8.596 (123.2) | 7.978 (54.7) |
| CL_{R,156-168} (mL/h) | 10.66 (114.9) | 3.480 (152.2) | 6.846 (93.4) | 4.674 (146.4) |

Abbreviations:
CL_{R,t1-t2} = renal clearance during the time interval specified by t1 and t2;
CumAe₀₋₁₆₈ = cumulative amount of BCX9250 excreted in the urine from time 0 to 168 hours;
Cum % excreted₀₋₁₆₈ = CumAe₀₋₁₆₈ expressed as a percentage of the total dose;
CV = coefficient of variation;
N = sample size;
ND = not done;
PK = pharmacokinetic
ª All data reported as geometric mean (CV % of geometric mean).

The results from Part 2 show oral dosing with Compound A was safe and generally well tolerated following multiple doses of 5, 10, 15, and 20 mg over 7 days. Safety assessments including TEAEs, clinical laboratory evaluations, vital signs, and bedside 12-lead ECG evaluations were overall unremarkable. Oral dosing with Compound A demonstrated linear and dose-proportional exposure over the sampling period and achieved drug expose (AUCₜₐᵤ) after 7 days QD administration of 20 mg in the range shown to suppress HO in a nonclinical model of activity of orally dosed Compound A.

### Pharmacodynamic Results for Parts 1 and 2

A secondary objective of Part 1 and Part 2 was to evaluate the effect of Compound A on the ECG intervals of interest and BP. Of particular interest was the assessment of potential correlations of ECG effects with Compound A plasma concentrations (PK/PD) over the 5 to 25 mg dose range in the single-dose study in Part 1 and the 5 to 20 mg dose range in the 7-day multiple-dose study in Part 2.

For the QTcF (QT interval corrected for heart rate by Fridericia's formula) assessment, most ΔQTcF (baseline-adjusted QTcF) and ΔΔQTcF (time-matched placebo- and baseline-adjusted QTcF) values were negative. No dose-response patterns were discernible for subjects dosed in Parts 1 and 2. The ΔQTcF-concentration model had a virtually flat slope (0.0002, P=0.9229 for difference from zero) and the fit line and its confidence boundaries fell well below 10 msec.

For the heart rate (HR) assessment, the change from baseline (CFB) was variably positive and negative for all treatment groups including placebo and a similar circadian change in ΔHR (baseline-adjusted HR) was observed. The mean ΔΔHR (baseline-adjusted, placebo corrected HR) was small at all time points and predominantly negative, which is primarily due to a ΔHR for placebo that was predominantly more positive than the ΔHR for Compound A. The ΔHR-concentration model, which included placebo, had a virtually flat slope (0.0004, P=0.8438 for difference from zero). The model predicted HR changes <1 bpm at all concentrations.

For the PR interval assessment, the mean ΔΔPR (baseline-adjusted, placebo-corrected PR interval) was small at all time points and predominantly negative, which is primarily due to a ΔPR (baseline-adjusted PR interval) for placebo that was predominantly more positive than the ΔPR for Compound A. The ΔPR-concentration model, which included placebo, had a nearly flat slope (−0.0033, P=0.1984 for difference from zero). The model predicted PR changes less than 2 msec at all concentrations.

For the QRS assessment, the mean ΔΔQRS (baseline-adjusted, placebo-corrected QRS interval) was small at all time points and predominantly negative due to predominantly more positive placebo changepoints. The ΔQRS (baseline-adjusted QRS interval)-concentration model, which included placebo, had a virtually flat slope (0.0004, P=0.6121 for difference from zero). The model predicted very small positive ΔΔQRS changes at all plasma concentrations.

Abnormal diagnostic statements were generally very infrequent, and none were clinically significant.

Results from the continuous ECG assessment in the PD ECG population via 12-lead Holter monitoring are consistent with the absence of a clinically significant effect of Compound A on the ECG at single doses up to 25 mg and multiple daily doses over 7 days up to 20 mg.

The mean systolic and diastolic CFB BP values were predominantly negative in the combined Compound A-treated groups for both Parts 1 and 2 of the study. The CFB values for the placebo groups were also predominantly negative for both systolic and diastolic BP for both Parts 1 and 2 of the study. A consideration of the mean CFB systolic and diastolic BP-time profiles suggests that there is no meaningful correlation of BP effects with Compound A dose in either the SAD study in Part 1 or the 7-day MAD study in Part 2.

The exposure-response modeling of the relationship between changes in systolic and diastolic BP and plasma Compound A concentrations further confirmed no meaningful correlation of BP with Compound A exposure. The baseline-adjusted (Δ) systolic and Δ diastolic BP-concentration models, which included data from all cohorts and from placebo subjects, had nearly flat slopes (0.00686 and 0.00151 for systolic and diastolic BP, respectively). The models predicted minimal changes in baseline-adjusted, placebo-corrected (ΔΔ) systolic and ΔΔ diastolic BP over the assessed concentration range.

The mean CFB systolic and diastolic BP-time profiles, as well as exposure-response modeling of the relationship between changes in BP and plasma Compound A concentrations, suggest that there is no meaningful correlation of BP with Compound A dose or exposure.

### CONCLUSION

The present disclosure demonstrates clinical utility of Compound A, a potent inhibitor of human ALK2(R206H) and wtALK2, in treating and/or preventing FOP and other diseases and conditions whose treatment would benefit from ALK2 kinase inhibition.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An oral dosage form, comprising a compound selected from: 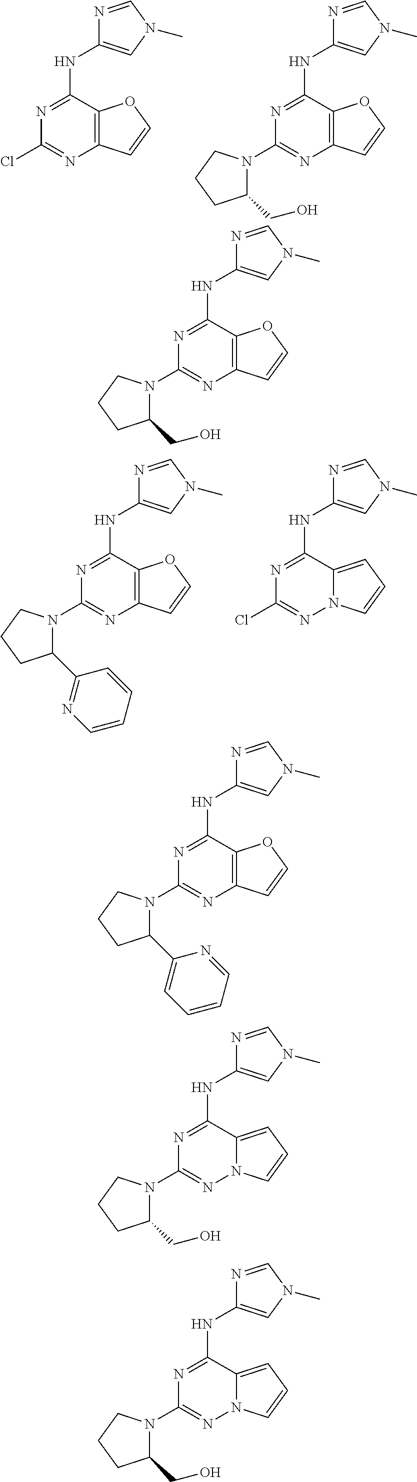 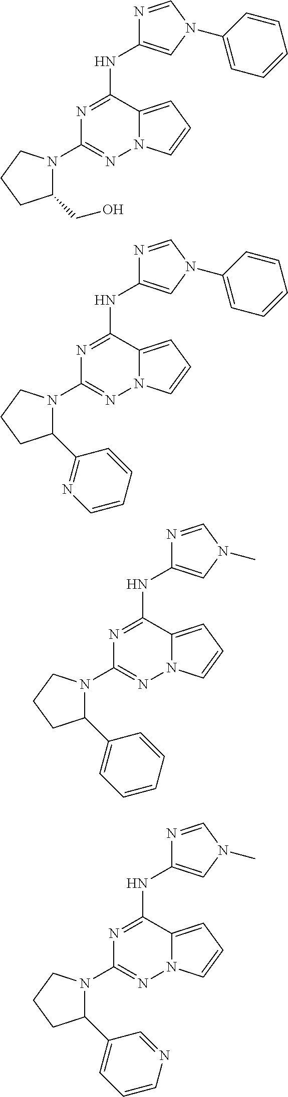 [SMILES: OC[C@@H]1CCCN1c1nc(Nc2cn(-c3ccccc3)cn2)c2cccn2n1 ; c1ccc(-n2cnc(Nc3nc(N4CCCC4c4ccccn4)nn4cccc34)c2)cc1 ; Cn1cnc(Nc2nc(N3CCCC3c3ccccc3)nn3cccc23)c1 ; Cn1cnc(Nc2nc(N3CCCC3c3cccnc3)nn3cccc23)c1] 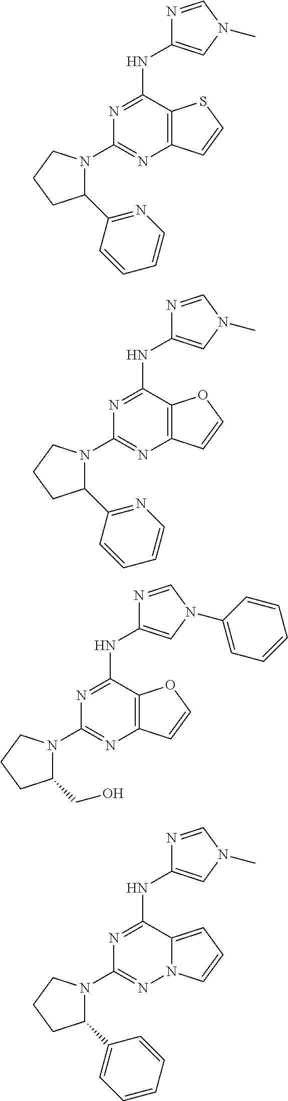 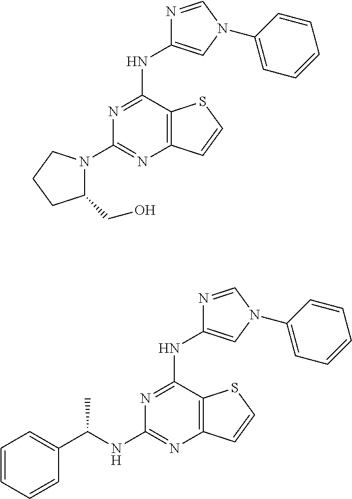 [SMILES: OC[C@@H]1CCCN1c1nc(Nc2cn(-c3ccccc3)cn2)c2sccc2n1 ; C[C@H](Nc1nc(Nc2cn(-c3ccccc3)cn2)c2sccc2n1)c1ccccc1] 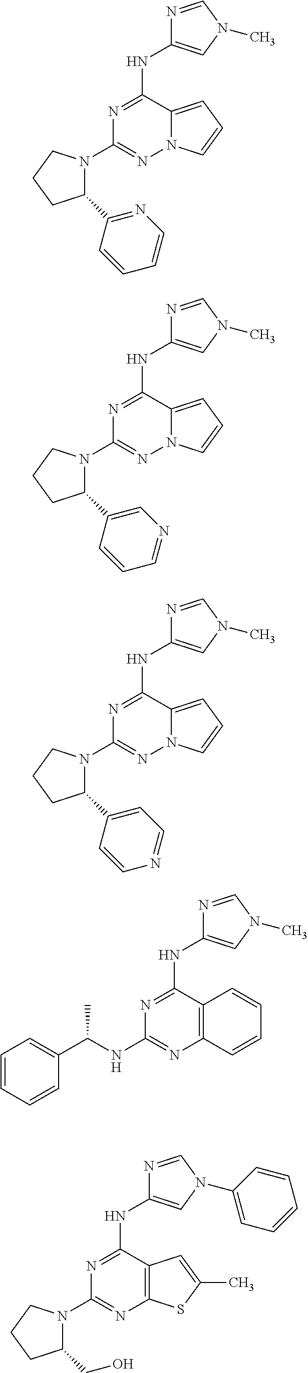 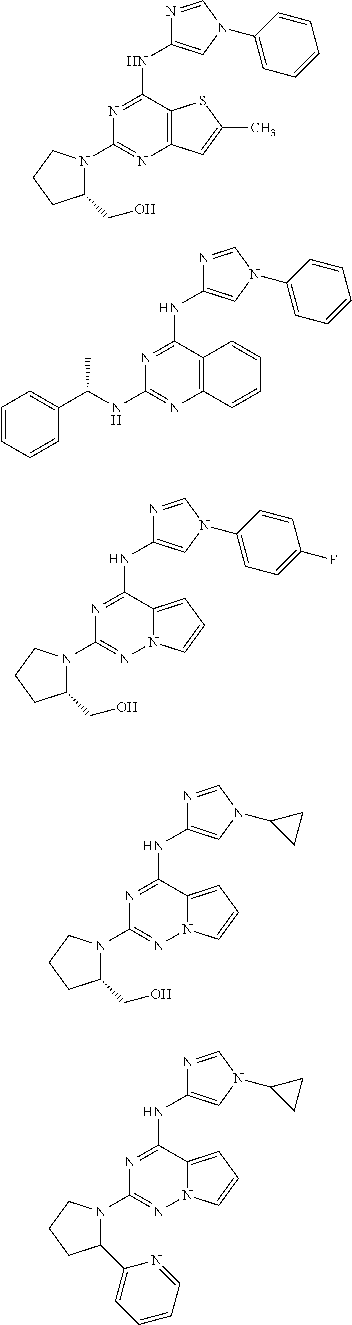 [SMILES: Cc1cc2nc(N3CCC[C@H]3CO)nc(Nc3cn(-c4ccccc4)cn3)c2s1 ; C[C@H](Nc1nc(Nc2cn(-c3ccccc3)cn2)c2ccccc2n1)c1ccccc1 ; OC[C@@H]1CCCN1c1nc(Nc2cn(-c3ccc(F)cc3)cn2)c2cccn2n1 ; OC[C@@H]1CCCN1c1nc(Nc2cn(C3CC3)cn2)c2cccn2n1 ; c1ccc(C2CCCN2c2nc(Nc3cn(C4CC4)cn3)c3cccn3n2)nc1] 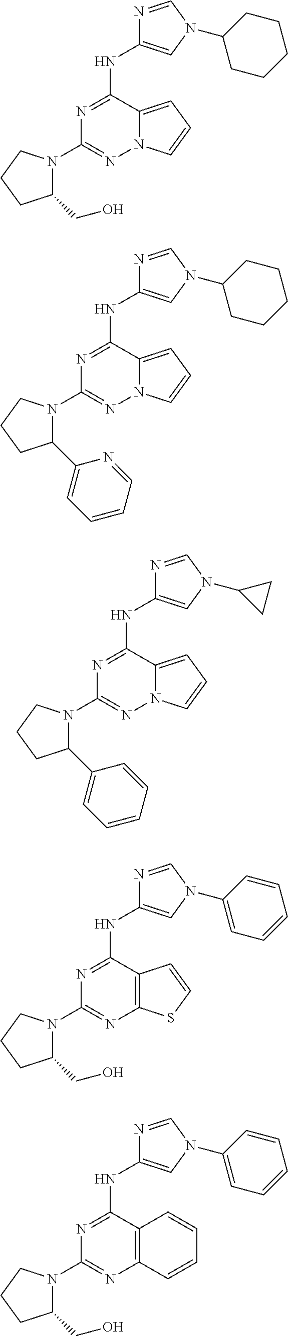 [SMILES: OC[C@@H]1CCCN1c1nc(Nc2cn(C3CCCCC3)cn2)c2cccn2n1 ; c1ccc(C2CCCN2c2nc(Nc3cn(C4CCCCC4)cn3)c3cccn3n2)nc1 ; c1ccc(C2CCCN2c2nc(Nc3cn(C4CC4)cn3)c3cccn3n2)cc1 ; OC[C@@H]1CCCN1c1nc(Nc2cn(-c3ccccc3)cn2)c2ccsc2n1 ; OC[C@@H]1CCCN1c1nc(Nc2cn(-c3ccccc3)cn2)c2ccccc2n1] 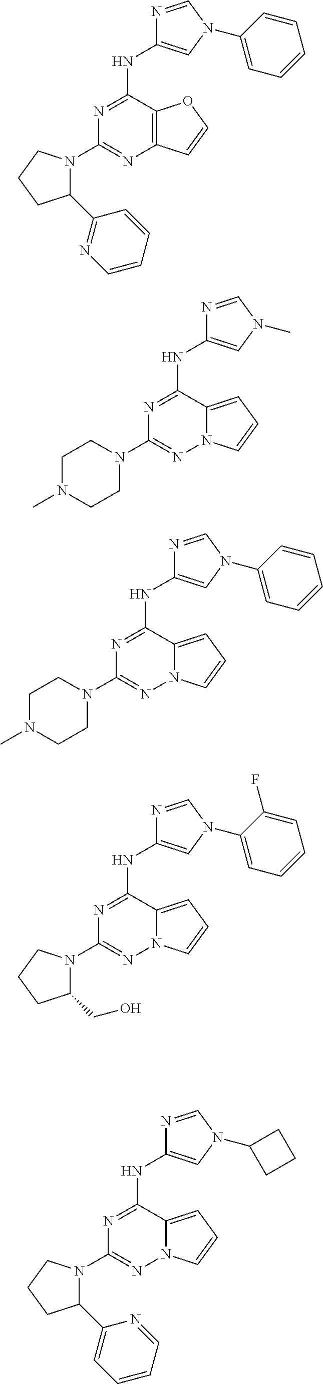 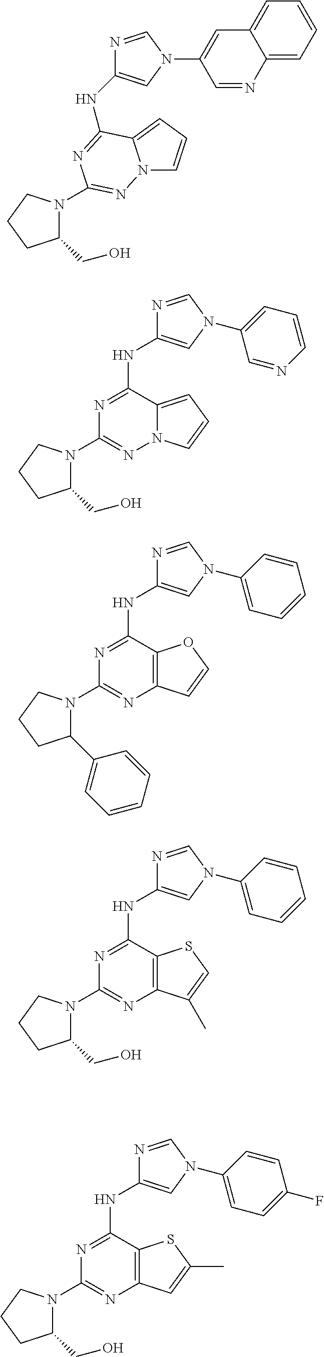 [SMILES: OC[C@@H]1CCCN1c1nc(Nc2cn(-c3cnc4ccccc4c3)cn2)c2cccn2n1 ; OC[C@@H]1CCCN1c1nc(Nc2cn(-c3cccnc3)cn2)c2cccn2n1 ; c1ccc(C2CCCN2c2nc(Nc3cn(-c4ccccc4)cn3)c3occc3n2)cc1 ; Cc1csc2c(Nc3cn(-c4ccccc4)cn3)nc(N3CCC[C@H]3CO)nc12 ; Cc1cc2nc(N3CCC[C@H]3CO)nc(Nc3cn(-c4ccc(F)cc4)cn3)c2s1] 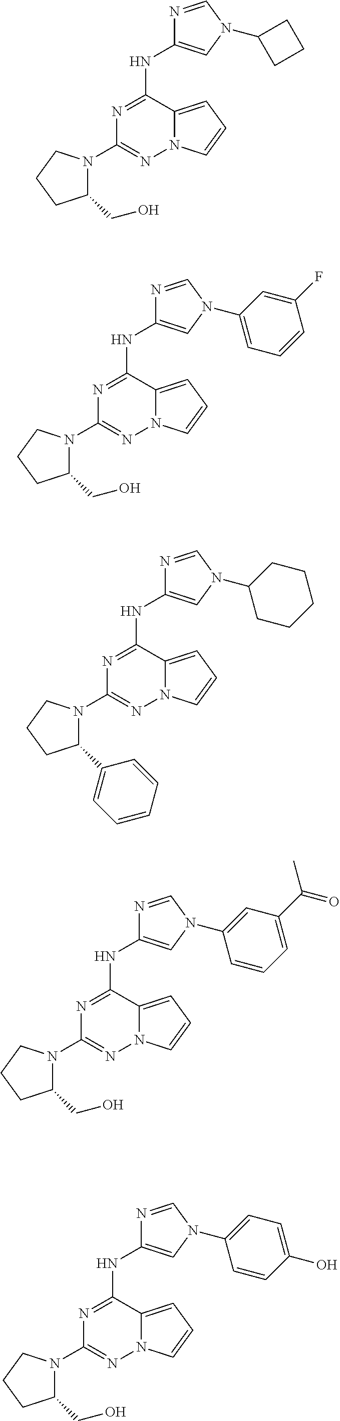 [SMILES: OC[C@@H]1CCCN1c1nc(Nc2cn(C3CCC3)cn2)c2cccn2n1 ; OC[C@@H]1CCCN1c1nc(Nc2cn(-c3cccc(F)c3)cn2)c2cccn2n1 ; c1ccc([C@@H]2CCCN2c2nc(Nc3cn(C4CCCCC4)cn3)c3cccn3n2)cc1 ; CC(=O)c1cccc(-n2cnc(Nc3nc(N4CCC[C@H]4CO)nn4cccc34)c2)c1 ; OC[C@@H]1CCCN1c1nc(Nc2cn(-c3ccc(O)cc3)cn2)c2cccn2n1] 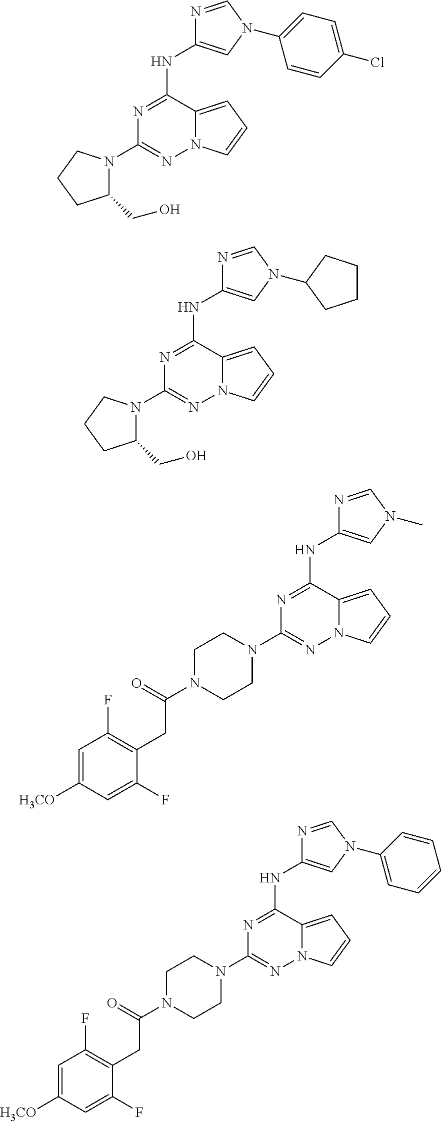 [SMILES: OC[C@@H]1CCCN1c1nc(Nc2cn(-c3ccc(Cl)cc3)cn2)c2cccn2n1 ; OC[C@@H]1CCCN1c1nc(Nc2cn(C3CCCC3)cn2)c2cccn2n1 ; COc1cc(F)c(CC(=O)N2CCN(c3nc(Nc4cn(C)cn4)c4cccn4n3)CC2)c(F)c1 ; COc1cc(F)c(CC(=O)N2CCN(c3nc(Nc4cn(-c5ccccc5)cn4)c4cccn4n3)CC2)c(F)c1] 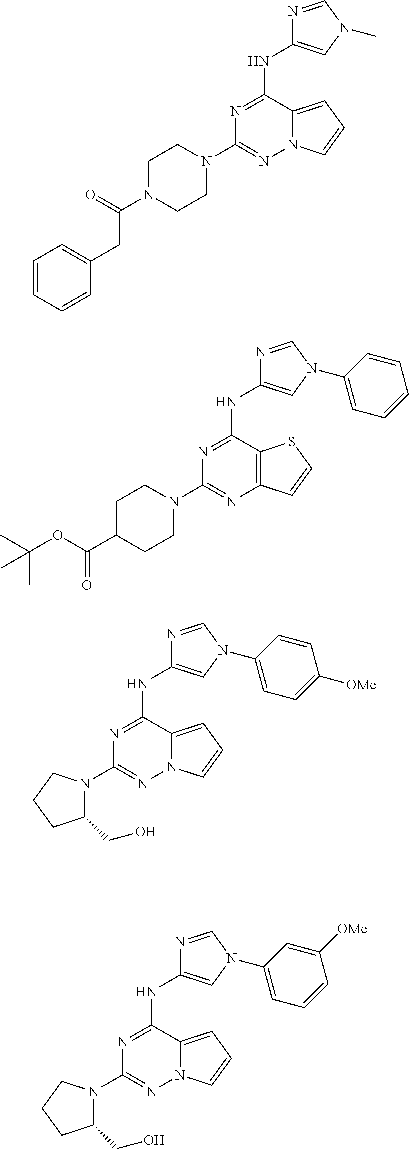 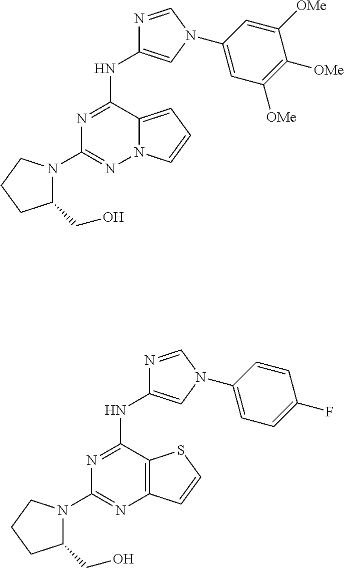 [SMILES: COc1cc(-n2cnc(Nc3nc(N4CCC[C@H]4CO)nn4cccc34)c2)cc(OC)c1OC ; OC[C@@H]1CCCN1c1nc(Nc2cn(-c3ccc(F)cc3)cn2)c2sccc2n1] 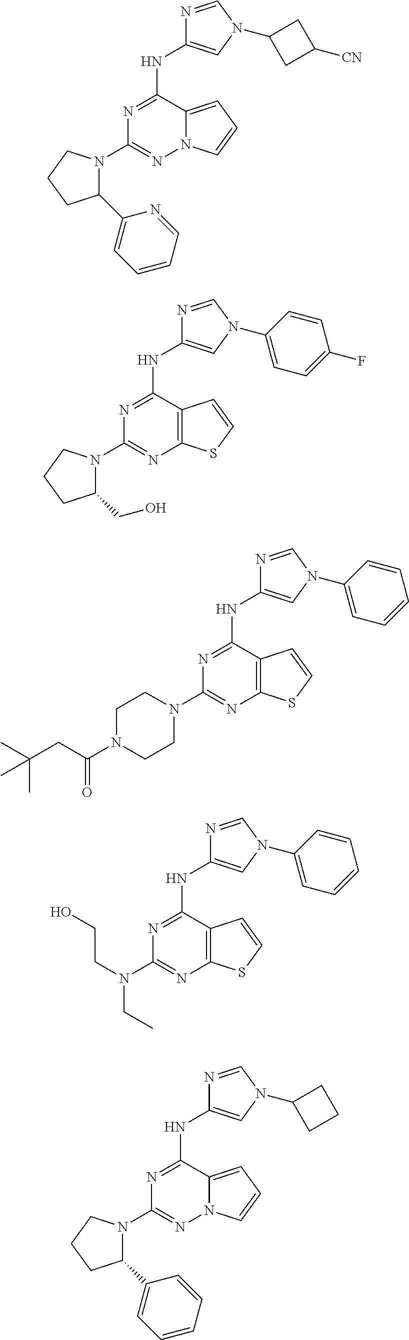 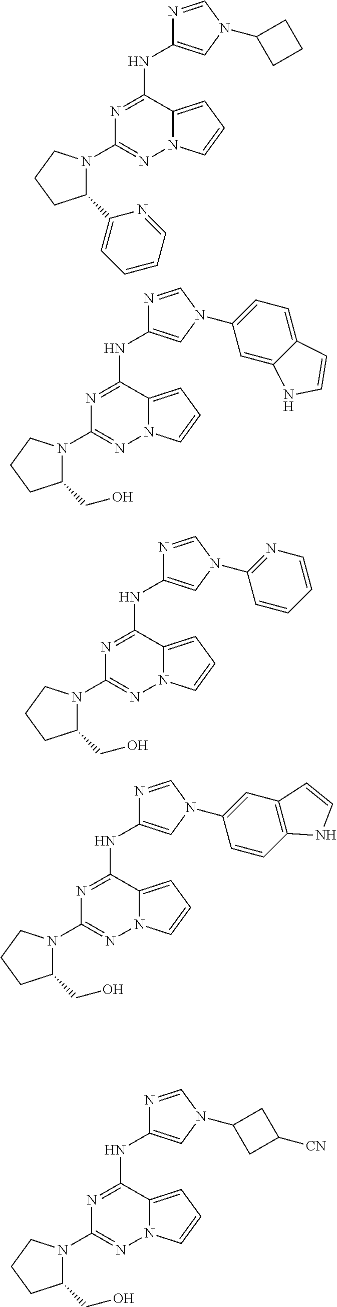 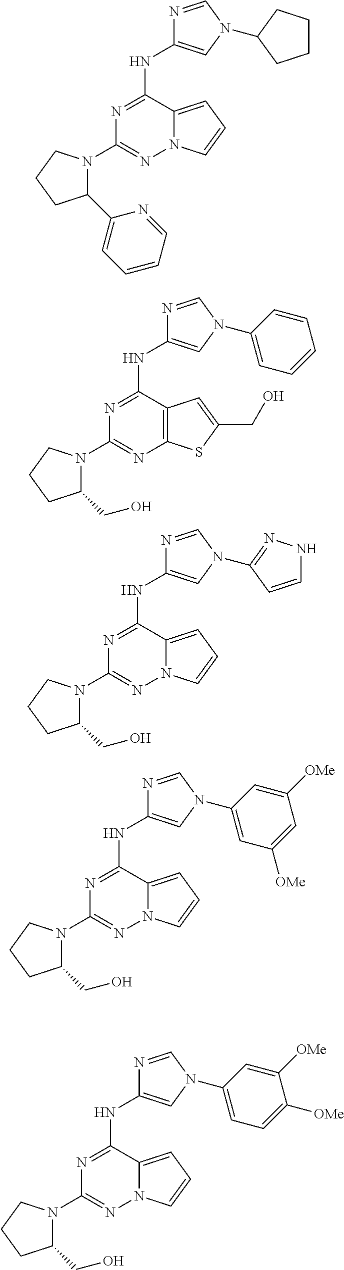 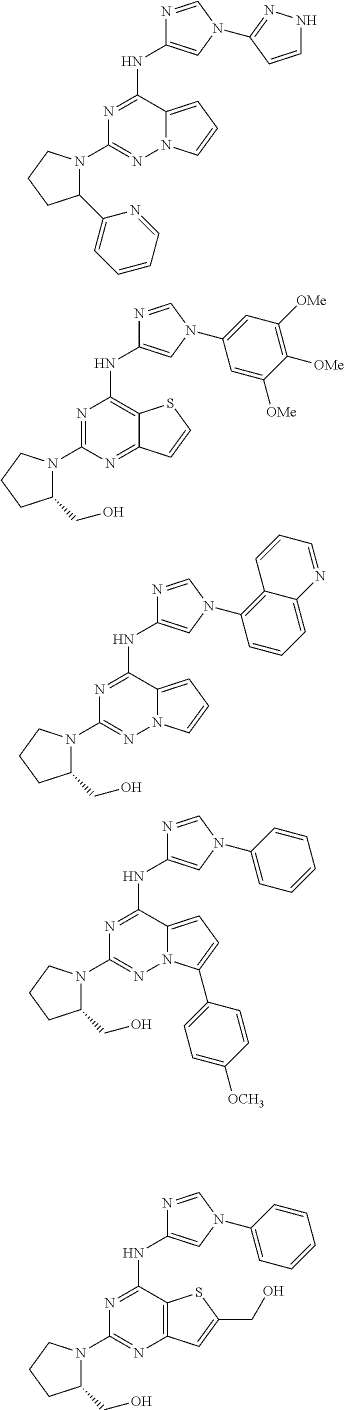 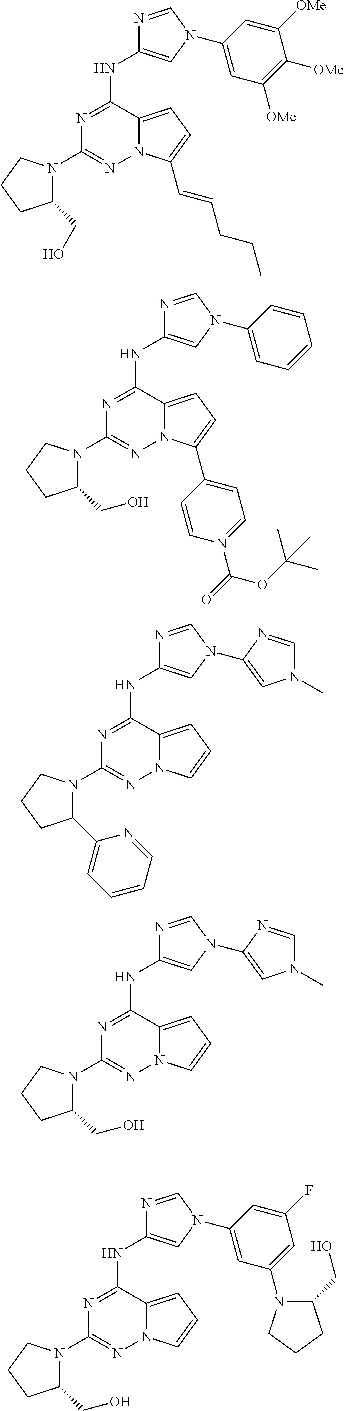 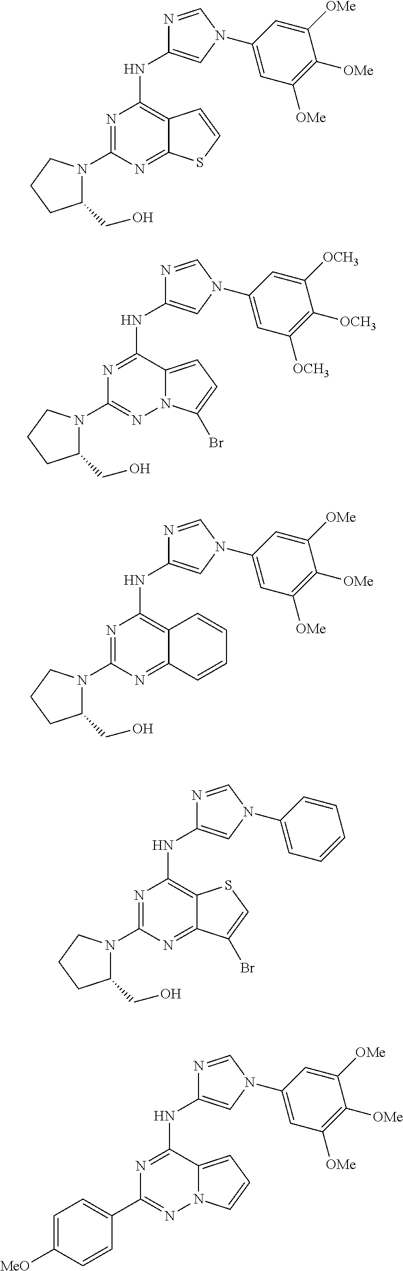 [SMILES: COc1cc(-n2cnc(Nc3nc(N4CCC[C@H]4CO)nc4sccc34)c2)cc(OC)c1OC ; COc1cc(-n2cnc(Nc3nc(N4CCC[C@H]4CO)nn4c(Br)ccc34)c2)cc(OC)c1OC ; COc1cc(-n2cnc(Nc3nc(N4CCC[C@H]4CO)nc4ccccc34)c2)cc(OC)c1OC ; OC[C@@H]1CCCN1c1nc(Nc2cn(-c3ccccc3)cn2)c2scc(Br)c2n1 ; COc1ccc(-c2nc(Nc3cn(-c4cc(OC)c(OC)c(OC)c4)cn3)c3cccn3n2)cc1] 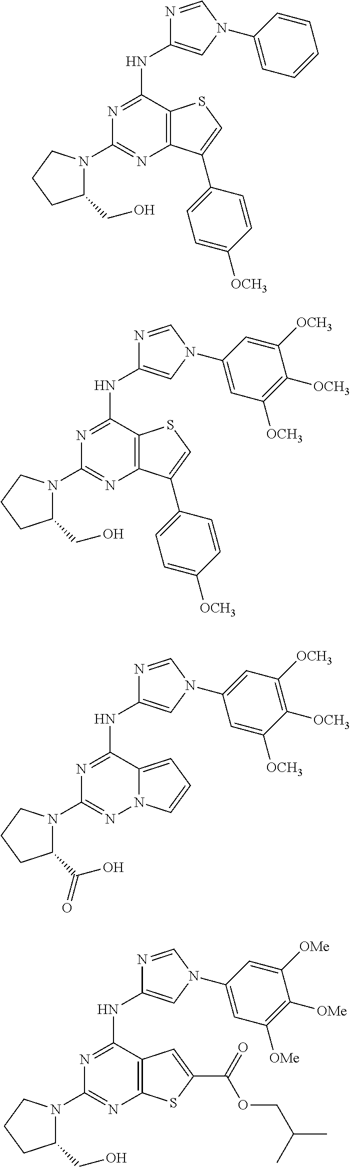 [SMILES: COc1ccc(-c2csc3c(Nc4cn(-c5ccccc5)cn4)nc(N4CCC[C@H]4CO)nc23)cc1 ; COc1ccc(-c2csc3c(Nc4cn(-c5cc(OC)c(OC)c(OC)c5)cn4)nc(N4CCC[C@H]4CO)nc23)cc1 ; COc1cc(-n2cnc(Nc3nc(N4CCC[C@H]4C(=O)O)nn4cccc34)c2)cc(OC)c1OC ; COc1cc(-n2cnc(Nc3nc(N4CCC[C@H]4CO)nc4sc(C(=O)OCC(C)C)cc34)c2)cc(OC)c1OC] 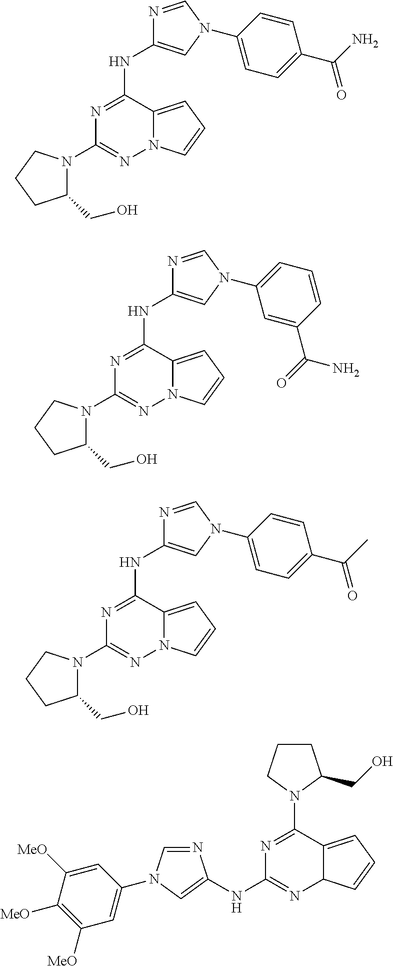 [SMILES: NC(=O)c1ccc(-n2cnc(Nc3nc(N4CCC[C@H]4CO)nn4cccc34)c2)cc1 ; NC(=O)c1cccc(-n2cnc(Nc3nc(N4CCC[C@H]4CO)nn4cccc34)c2)c1 ; CC(=O)c1ccc(-n2cnc(Nc3nc(N4CCC[C@H]4CO)nn4cccc34)c2)cc1 ; COc1cc(-n2cnc(NC3=NC4C=CC=C4C(N4CCC[C@H]4CO)=N3)c2)cc(OC)c1OC] 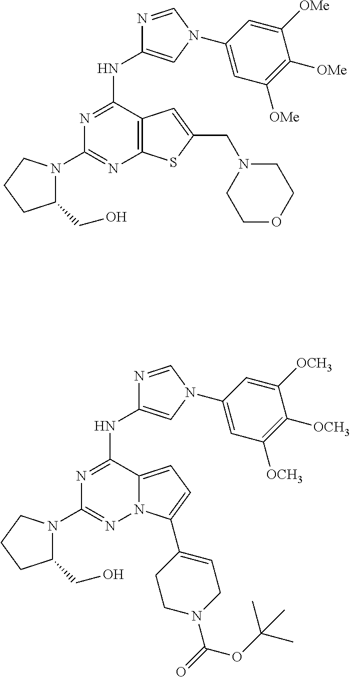 [SMILES: COc1cc(-n2cnc(Nc3nc(N4CCC[C@H]4CO)nc4sc(CN5CCOCC5)cc34)c2)cc(OC)c1OC ; COc1cc(-n2cnc(Nc3nc(N4CCC[C@H]4CO)nn4c(C5=CCN(C(=O)OC(C)(C)C)CC5)ccc34)c2)cc(OC)c1OC] 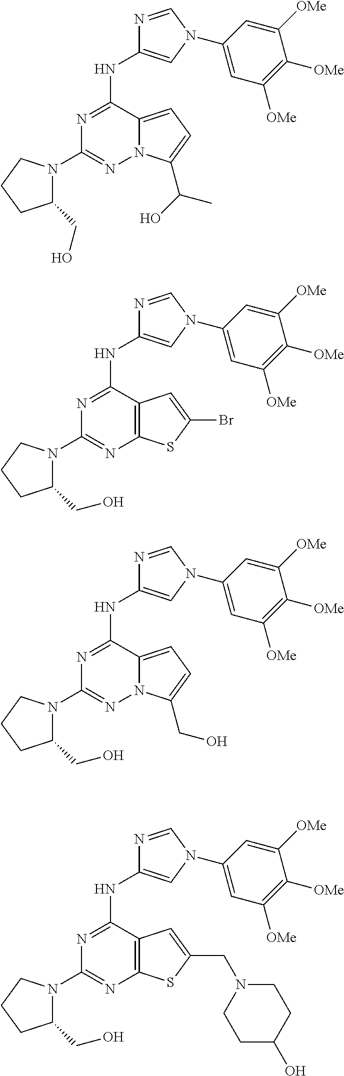 [SMILES: COc1cc(-n2cnc(Nc3nc(N4CCC[C@H]4CO)nn4c(C(C)O)ccc34)c2)cc(OC)c1OC ; COc1cc(-n2cnc(Nc3nc(N4CCC[C@H]4CO)nc4sc(Br)cc34)c2)cc(OC)c1OC ; COc1cc(-n2cnc(Nc3nc(N4CCC[C@H]4CO)nn4c(CO)ccc34)c2)cc(OC)c1OC ; COc1cc(-n2cnc(Nc3nc(N4CCC[C@H]4CO)nc4sc(CN5CCC(O)CC5)cc34)c2)cc(OC)c1OC] 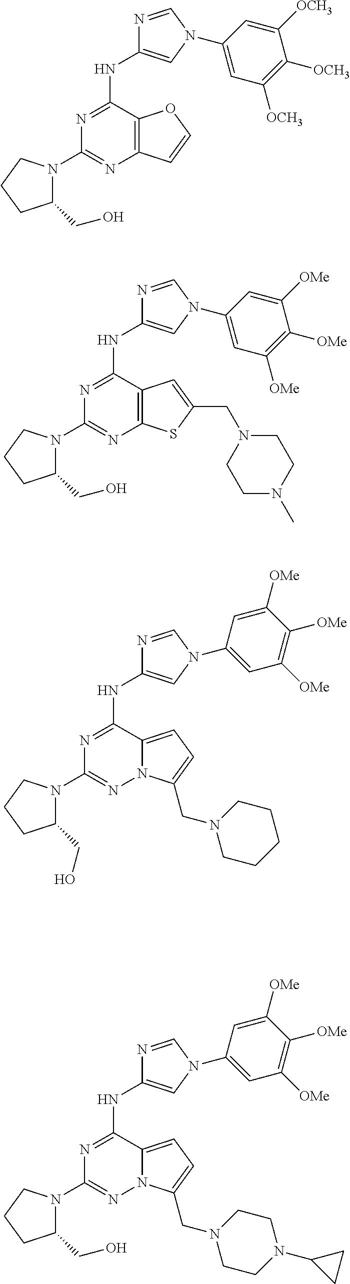 [SMILES: COc1cc(-n2cnc(Nc3nc(N4CCC[C@H]4CO)nc4ccoc34)c2)cc(OC)c1OC ; COc1cc(-n2cnc(Nc3nc(N4CCC[C@H]4CO)nc4sc(CN5CCN(C)CC5)cc34)c2)cc(OC)c1OC ; COc1cc(-n2cnc(Nc3nc(N4CCC[C@H]4CO)nn4c(CN5CCCCC5)ccc34)c2)cc(OC)c1OC ; COc1cc(-n2cnc(Nc3nc(N4CCC[C@H]4CO)nn4c(CN5CCN(C6CC6)CC5)ccc34)c2)cc(OC)c1OC] 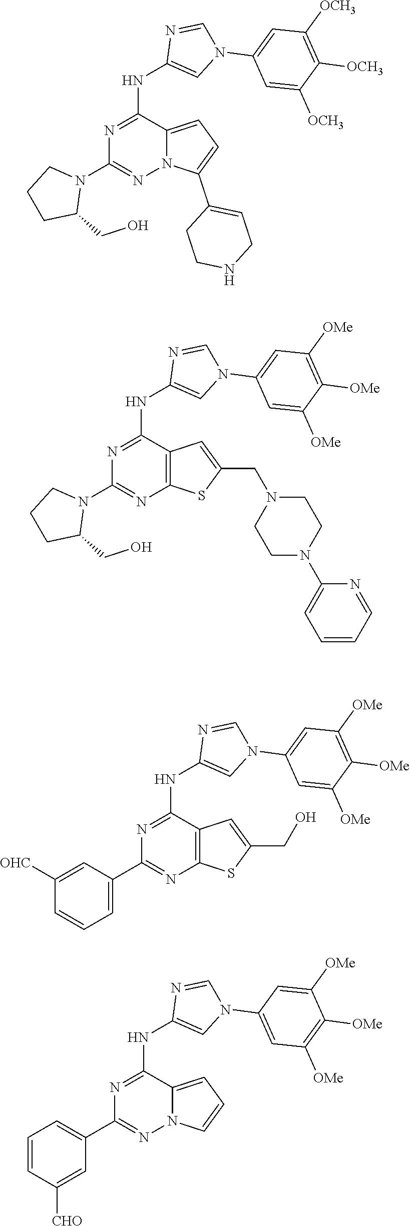 [SMILES: COc1cc(-n2cnc(Nc3nc(N4CCC[C@H]4CO)nn4c(C5=CCNCC5)ccc34)c2)cc(OC)c1OC ; COc1cc(-n2cnc(Nc3nc(N4CCC[C@H]4CO)nc4sc(CN5CCN(c6ccccn6)CC5)cc34)c2)cc(OC)c1OC ; COc1cc(-n2cnc(Nc3nc(-c4cccc(C=O)c4)nc4sc(CO)cc34)c2)cc(OC)c1OC ; COc1cc(-n2cnc(Nc3nc(-c4cccc(C=O)c4)nn4cccc34)c2)cc(OC)c1OC] 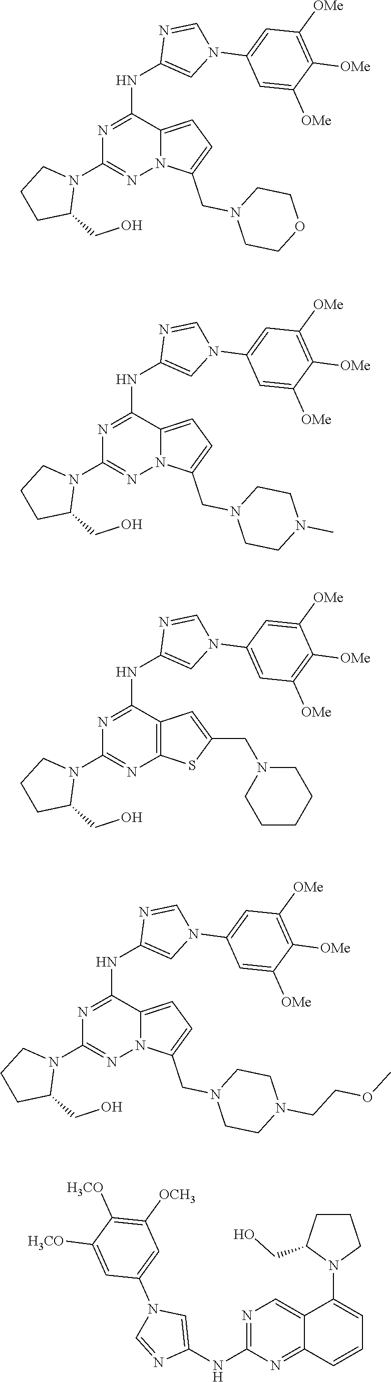 [SMILES: COc1cc(-n2cnc(Nc3nc(N4CCC[C@H]4CO)nn4c(CN5CCOCC5)ccc34)c2)cc(OC)c1OC ; COc1cc(-n2cnc(Nc3nc(N4CCC[C@H]4CO)nn4c(CN5CCN(C)CC5)ccc34)c2)cc(OC)c1OC ; COc1cc(-n2cnc(Nc3nc(N4CCC[C@H]4CO)nc4sc(CN5CCCCC5)cc34)c2)cc(OC)c1OC ; COCCN1CCN(Cc2ccc3c(Nc4cn(-c5cc(OC)c(OC)c(OC)c5)cn4)nc(N4CCC[C@H]4CO)nn23)CC1 ; COc1cc(-n2cnc(Nc3ncc4c(N5CCC[C@H]5CO)cccc4n3)c2)cc(OC)c1OC] 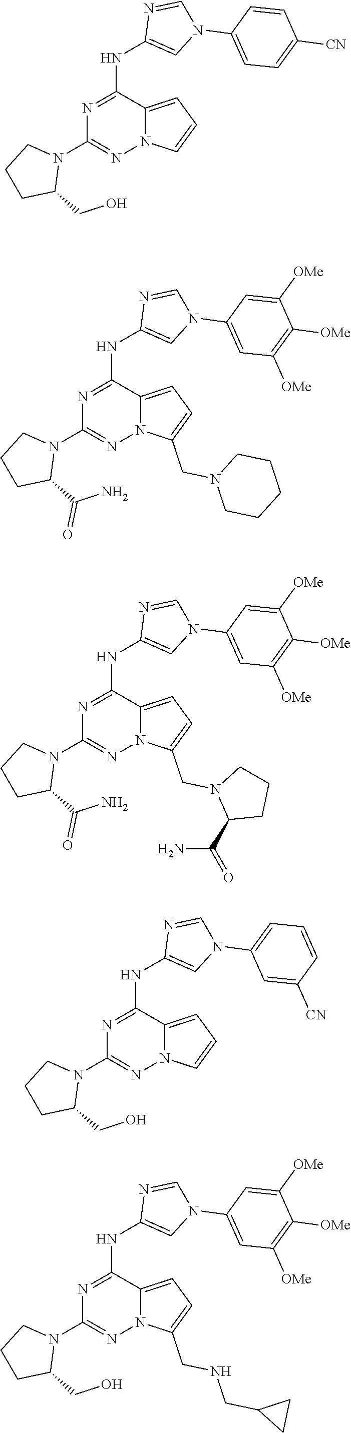 [SMILES: N#Cc1ccc(-n2cnc(Nc3nc(N4CCC[C@H]4CO)nn4cccc34)c2)cc1 ; COc1cc(-n2cnc(Nc3nc(N4CCC[C@H]4C(N)=O)nn4c(CN5CCCCC5)ccc34)c2)cc(OC)c1OC ; COc1cc(-n2cnc(Nc3nc(N4CCC[C@H]4C(N)=O)nn4c(CN5CCC[C@H]5C(N)=O)ccc34)c2)cc(OC)c1OC ; N#Cc1cccc(-n2cnc(Nc3nc(N4CCC[C@H]4CO)nn4cccc34)c2)c1 ; COc1cc(-n2cnc(Nc3nc(N4CCC[C@H]4CO)nn4c(CNCC5CC5)ccc34)c2)cc(OC)c1OC] 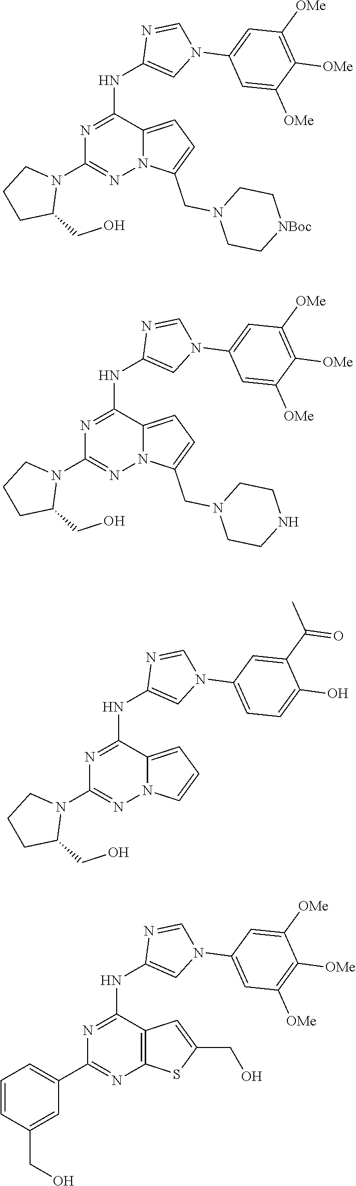 [SMILES: COc1cc(-n2cnc(Nc3nc(N4CCC[C@H]4CO)nn4c(CN5CCN(C(=O)OC(C)(C)C)CC5)ccc34)c2)cc(OC)c1OC ; COc1cc(-n2cnc(Nc3nc(N4CCC[C@H]4CO)nn4c(CN5CCNCC5)ccc34)c2)cc(OC)c1OC ; CC(=O)c1cc(-n2cnc(Nc3nc(N4CCC[C@H]4CO)nn4cccc34)c2)ccc1O ; COc1cc(-n2cnc(Nc3nc(-c4cccc(CO)c4)nc4sc(CO)cc34)c2)cc(OC)c1OC] 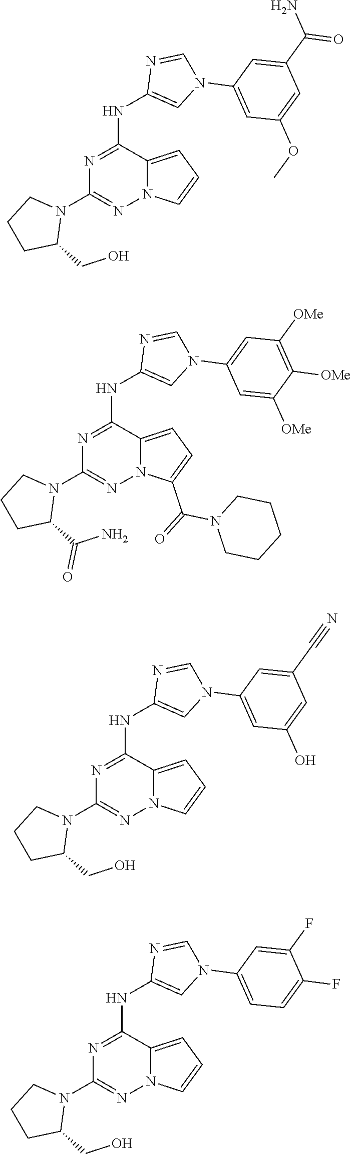 [SMILES: COc1cc(C(N)=O)cc(-n2cnc(Nc3nc(N4CCC[C@H]4CO)nn4cccc34)c2)c1 ; COc1cc(-n2cnc(Nc3nc(N4CCC[C@H]4C(N)=O)nn4c(C(=O)N5CCCCC5)ccc34)c2)cc(OC)c1OC ; N#Cc1cc(O)cc(-n2cnc(Nc3nc(N4CCC[C@H]4CO)nn4cccc34)c2)c1 ; OC[C@@H]1CCCN1c1nc(Nc2cn(-c3ccc(F)c(F)c3)cn2)c2cccn2n1] 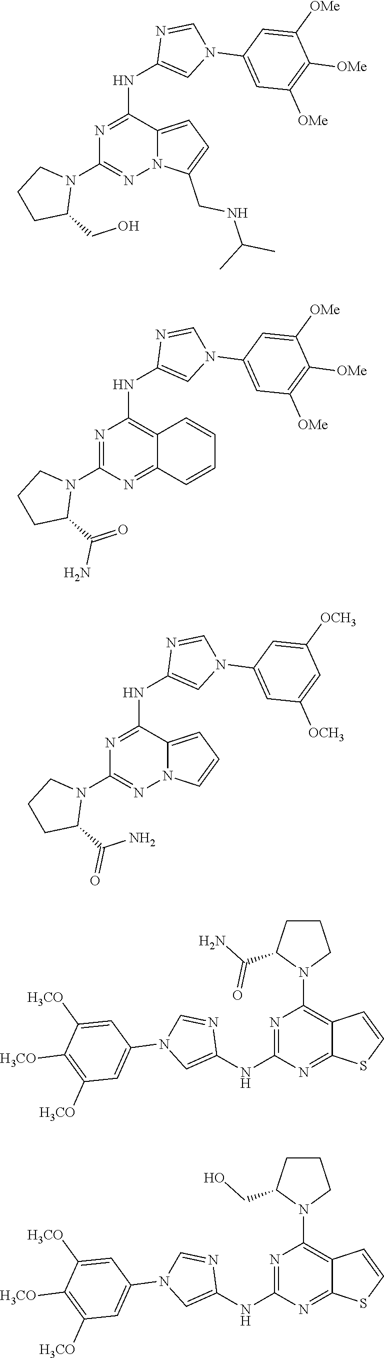 [SMILES: COc1cc(-n2cnc(Nc3nc(N4CCC[C@H]4CO)nn4c(CNC(C)C)ccc34)c2)cc(OC)c1OC ; COc1cc(-n2cnc(Nc3nc(N4CCC[C@H]4C(N)=O)nc4ccccc34)c2)cc(OC)c1OC ; COc1cc(OC)cc(-n2cnc(Nc3nc(N4CCC[C@H]4C(N)=O)nn4cccc34)c2)c1 ; COc1cc(-n2cnc(Nc3nc(N4CCC[C@H]4C(N)=O)c4ccsc4n3)c2)cc(OC)c1OC ; COc1cc(-n2cnc(Nc3nc(N4CCC[C@H]4CO)c4ccsc4n3)c2)cc(OC)c1OC] 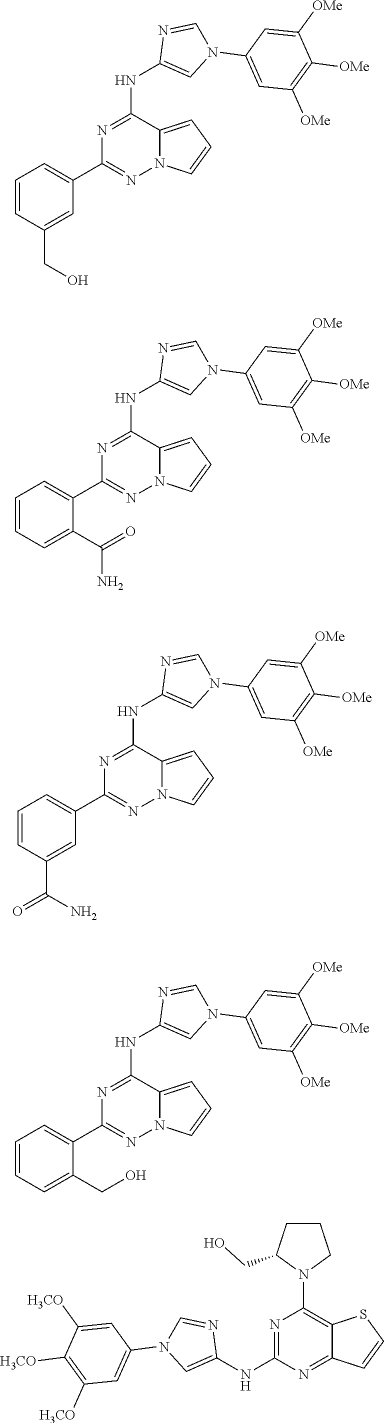 [SMILES: COc1cc(-n2cnc(Nc3nc(-c4cccc(CO)c4)nn4cccc34)c2)cc(OC)c1OC ; COc1cc(-n2cnc(Nc3nc(-c4ccccc4C(N)=O)nn4cccc34)c2)cc(OC)c1OC ; COc1cc(-n2cnc(Nc3nc(-c4cccc(C(N)=O)c4)nn4cccc34)c2)cc(OC)c1OC ; COc1cc(-n2cnc(Nc3nc(-c4ccccc4CO)nn4cccc34)c2)cc(OC)c1OC ; COc1cc(-n2cnc(Nc3nc(N4CCC[C@H]4CO)c4sccc4n3)c2)cc(OC)c1OC] 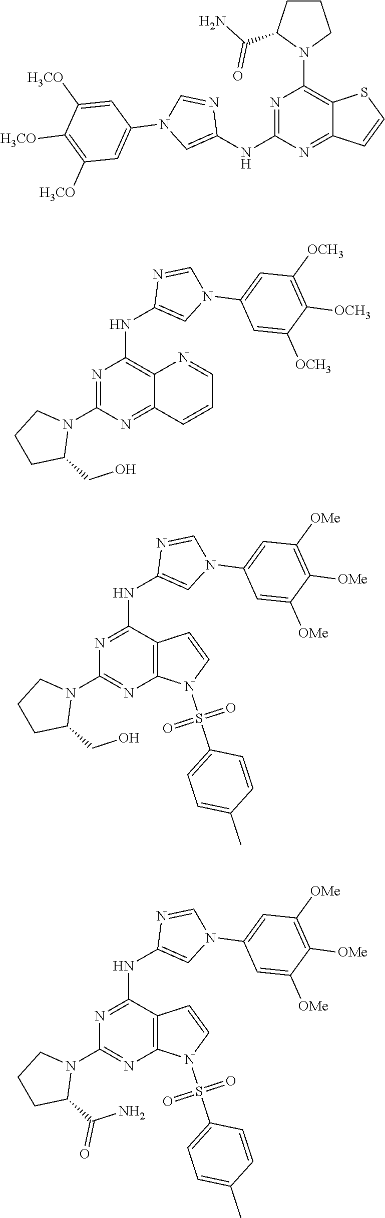 [SMILES: COc1cc(-n2cnc(Nc3nc(N4CCC[C@H]4C(N)=O)c4sccc4n3)c2)cc(OC)c1OC ; COc1cc(-n2cnc(Nc3nc(N4CCC[C@H]4CO)nc4cccnc34)c2)cc(OC)c1OC ; COc1cc(-n2cnc(Nc3nc(N4CCC[C@H]4CO)nc4c3ccn4S(=O)(=O)c3ccc(C)cc3)c2)cc(OC)c1OC ; COc1cc(-n2cnc(Nc3nc(N4CCC[C@H]4C(N)=O)nc4c3ccn4S(=O)(=O)c3ccc(C)cc3)c2)cc(OC)c1OC] 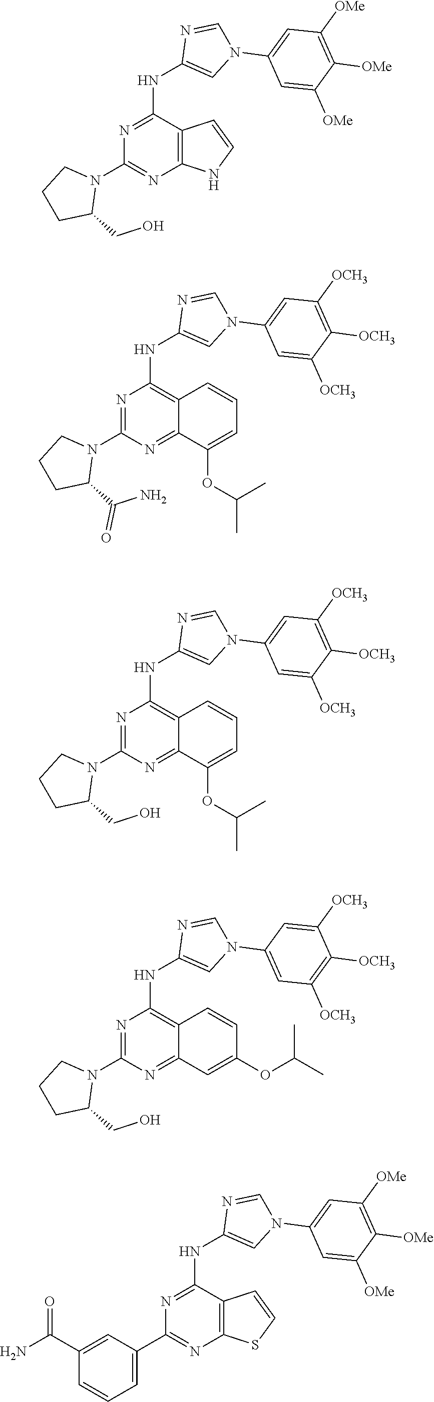 [SMILES: COc1cc(-n2cnc(Nc3nc(N4CCC[C@H]4CO)nc4[nH]ccc34)c2)cc(OC)c1OC ; COc1cc(-n2cnc(Nc3nc(N4CCC[C@H]4C(N)=O)nc4c(OC(C)C)cccc34)c2)cc(OC)c1OC ; COc1cc(-n2cnc(Nc3nc(N4CCC[C@H]4CO)nc4c(OC(C)C)cccc34)c2)cc(OC)c1OC ; COc1cc(-n2cnc(Nc3nc(N4CCC[C@H]4CO)nc4cc(OC(C)C)ccc34)c2)cc(OC)c1OC ; COc1cc(-n2cnc(Nc3nc(-c4cccc(C(N)=O)c4)nc4sccc34)c2)cc(OC)c1OC] 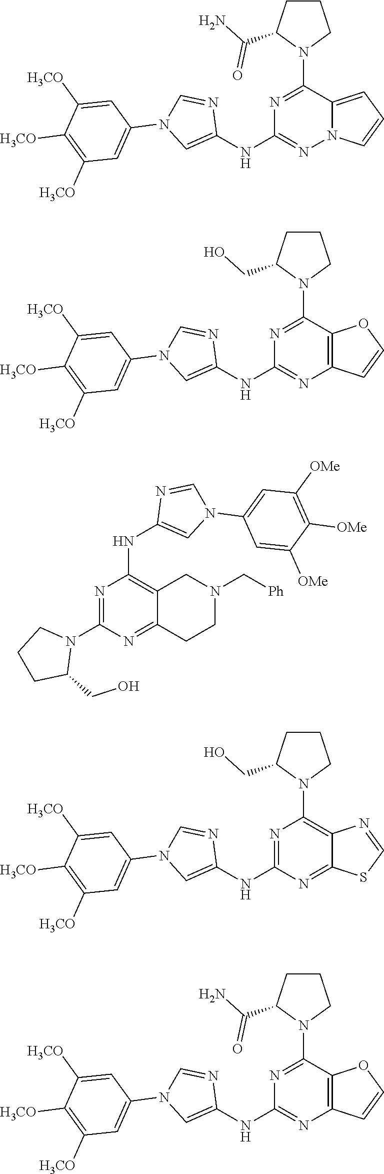 [SMILES: COc1cc(-n2cnc(Nc3nc(N4CCC[C@H]4C(N)=O)c4cccn4n3)c2)cc(OC)c1OC ; COc1cc(-n2cnc(Nc3nc(N4CCC[C@H]4CO)c4occc4n3)c2)cc(OC)c1OC ; COc1cc(-n2cnc(Nc3nc(N4CCC[C@H]4CO)nc4c3CN(Cc3ccccc3)CC4)c2)cc(OC)c1OC ; COc1cc(-n2cnc(Nc3nc(N4CCC[C@H]4CO)c4ncsc4n3)c2)cc(OC)c1OC ; COc1cc(-n2cnc(Nc3nc(N4CCC[C@H]4C(N)=O)c4occc4n3)c2)cc(OC)c1OC] 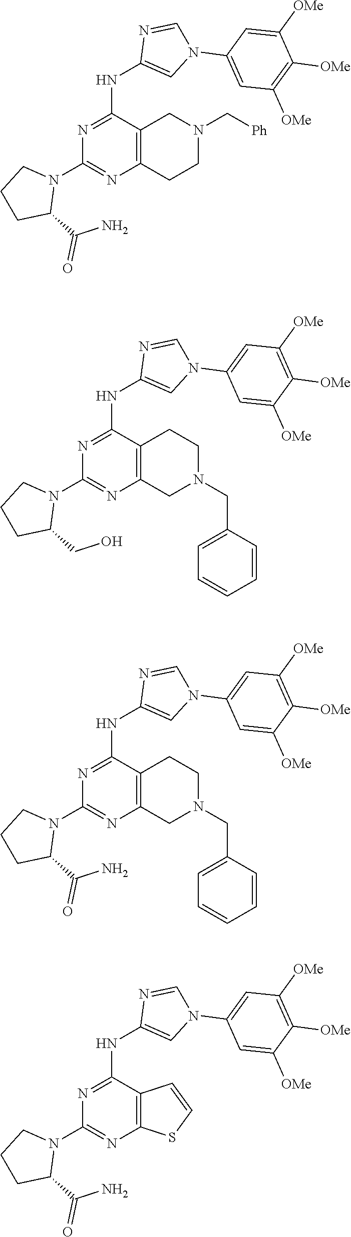 [SMILES: COc1cc(-n2cnc(Nc3nc(N4CCC[C@H]4C(N)=O)nc4c3CN(Cc3ccccc3)CC4)c2)cc(OC)c1OC ; COc1cc(-n2cnc(Nc3nc(N4CCC[C@H]4CO)nc4c3CCN(Cc3ccccc3)C4)c2)cc(OC)c1OC ; COc1cc(-n2cnc(Nc3nc(N4CCC[C@H]4C(N)=O)nc4c3CCN(Cc3ccccc3)C4)c2)cc(OC)c1OC ; COc1cc(-n2cnc(Nc3nc(N4CCC[C@H]4C(N)=O)nc4sccc34)c2)cc(OC)c1OC] 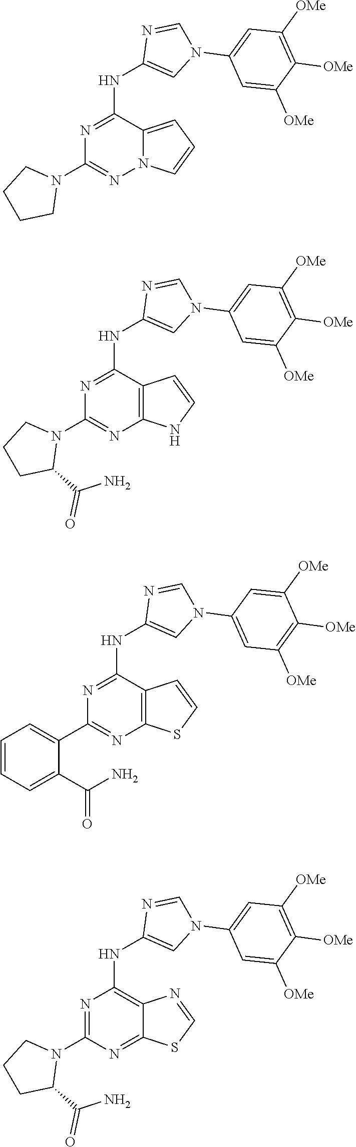 [SMILES: COc1cc(-n2cnc(Nc3nc(N4CCCC4)nn4cccc34)c2)cc(OC)c1OC ; COc1cc(-n2cnc(Nc3nc(N4CCC[C@H]4C(N)=O)nc4[nH]ccc34)c2)cc(OC)c1OC ; COc1cc(-n2cnc(Nc3nc(-c4ccccc4C(N)=O)nc4sccc34)c2)cc(OC)c1OC ; COc1cc(-n2cnc(Nc3nc(N4CCC[C@H]4C(N)=O)nc4scnc34)c2)cc(OC)c1OC] 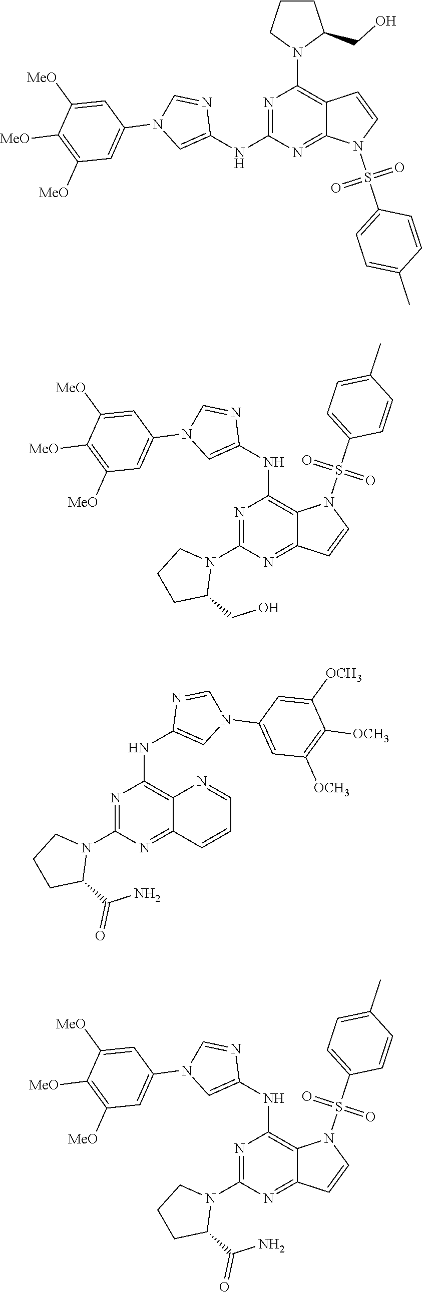 [SMILES: COc1cc(-n2cnc(Nc3nc(N4CCC[C@H]4CO)c4ccn(S(=O)(=O)c5ccc(C)cc5)c4n3)c2)cc(OC)c1OC ; COc1cc(-n2cnc(Nc3nc(N4CCC[C@H]4CO)nc4ccn(S(=O)(=O)c5ccc(C)cc5)c34)c2)cc(OC)c1OC ; COc1cc(-n2cnc(Nc3nc(N4CCC[C@H]4C(N)=O)nc4cccnc34)c2)cc(OC)c1OC ; COc1cc(-n2cnc(Nc3nc(N4CCC[C@H]4C(N)=O)nc4ccn(S(=O)(=O)c5ccc(C)cc5)c34)c2)cc(OC)c1OC] 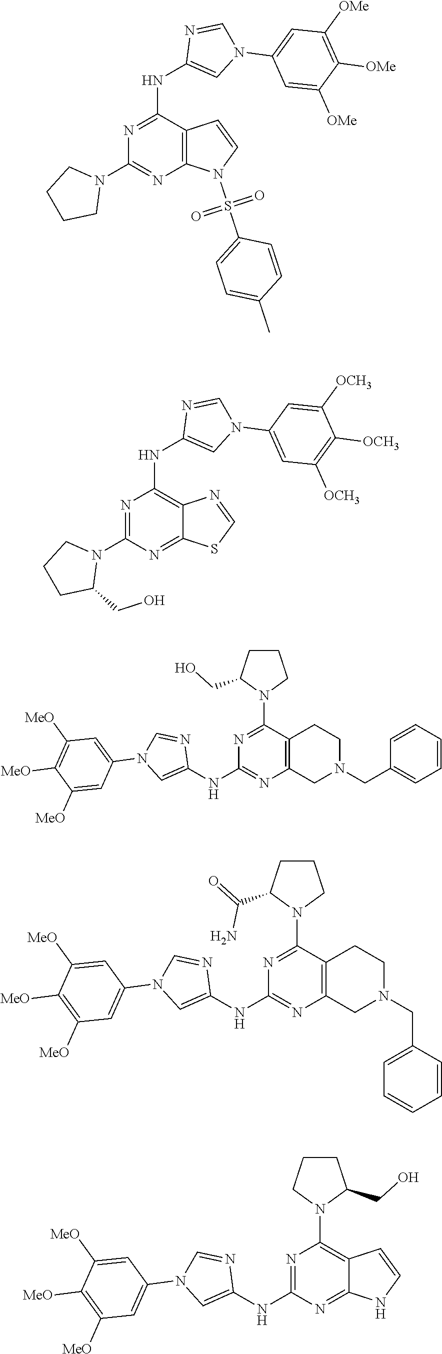 [SMILES: COc1cc(-n2cnc(Nc3nc(N4CCCC4)nc4c3ccn4S(=O)(=O)c3ccc(C)cc3)c2)cc(OC)c1OC ; COc1cc(-n2cnc(Nc3nc(N4CCC[C@H]4CO)nc4scnc34)c2)cc(OC)c1OC ; COc1cc(-n2cnc(Nc3nc4c(c(N5CCC[C@H]5CO)n3)CCN(Cc3ccccc3)C4)c2)cc(OC)c1OC ; COc1cc(-n2cnc(Nc3nc4c(c(N5CCC[C@H]5C(N)=O)n3)CCN(Cc3ccccc3)C4)c2)cc(OC)c1OC ; COc1cc(-n2cnc(Nc3nc(N4CCC[C@H]4CO)c4cc[nH]c4n3)c2)cc(OC)c1OC] 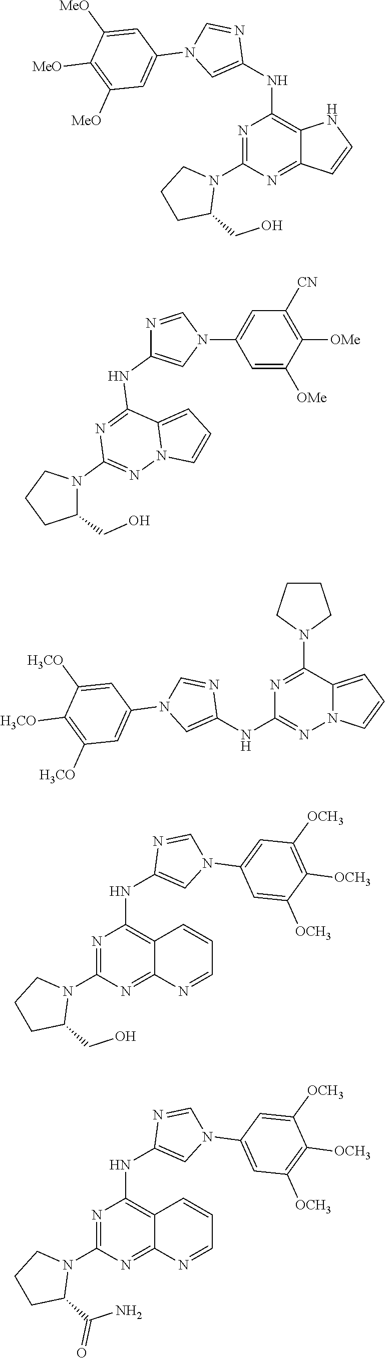 [SMILES: COc1cc(-n2cnc(Nc3nc(N4CCC[C@H]4CO)nc4cc[nH]c34)c2)cc(OC)c1OC ; COc1cc(-n2cnc(Nc3nc(N4CCC[C@H]4CO)nn4cccc34)c2)cc(C#N)c1OC ; COc1cc(-n2cnc(Nc3nc(N4CCCC4)c4cccn4n3)c2)cc(OC)c1OC ; COc1cc(-n2cnc(Nc3nc(N4CCC[C@H]4CO)nc4ncccc34)c2)cc(OC)c1OC ; COc1cc(-n2cnc(Nc3nc(N4CCC[C@H]4C(N)=O)nc4ncccc34)c2)cc(OC)c1OC] 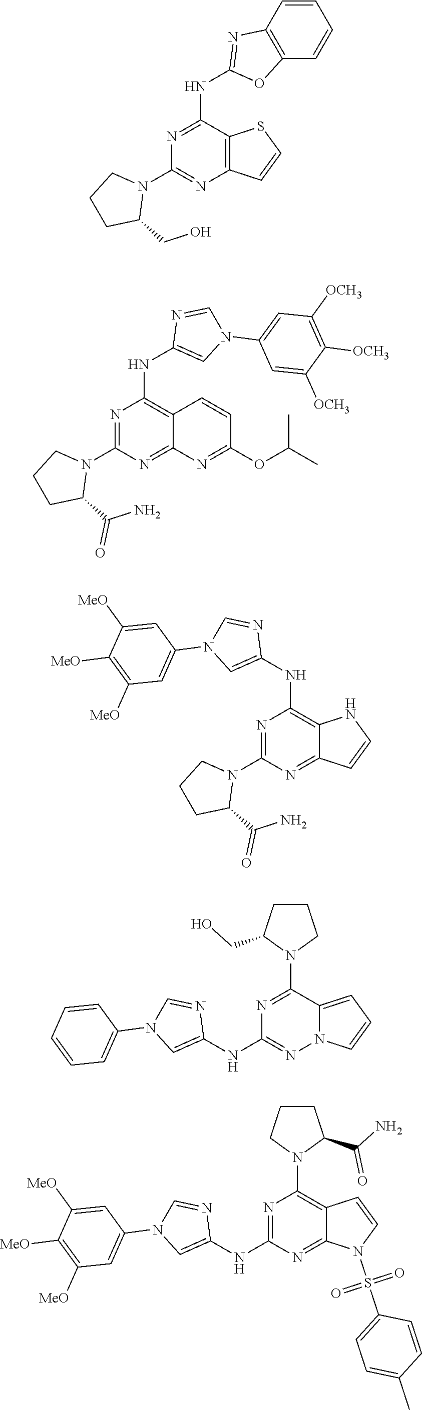 [SMILES: OC[C@@H]1CCCN1c1nc(Nc2nc3ccccc3o2)c2sccc2n1 ; COc1cc(-n2cnc(Nc3nc(N4CCC[C@H]4C(N)=O)nc4nc(OC(C)C)ccc34)c2)cc(OC)c1OC ; COc1cc(-n2cnc(Nc3nc(N4CCC[C@H]4C(N)=O)nc4cc[nH]c34)c2)cc(OC)c1OC ; OC[C@@H]1CCCN1c1nc(Nc2cn(-c3ccccc3)cn2)nn2cccc12 ; COc1cc(-n2cnc(Nc3nc(N4CCC[C@H]4C(N)=O)c4ccn(S(=O)(=O)c5ccc(C)cc5)c4n3)c2)cc(OC)c1OC] 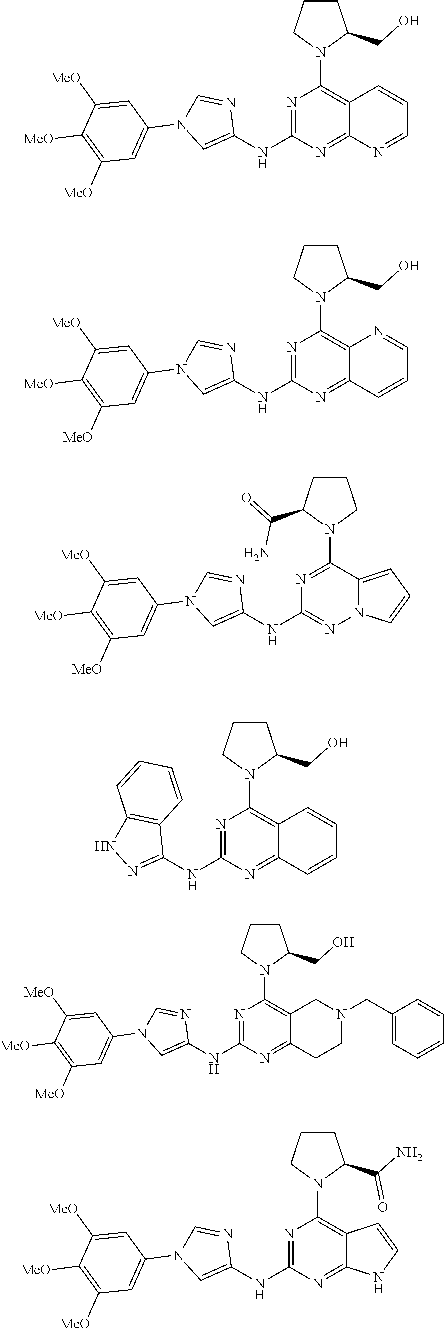 [SMILES: COc1cc(-n2cnc(Nc3nc(N4CCC[C@H]4CO)c4cccnc4n3)c2)cc(OC)c1OC ; COc1cc(-n2cnc(Nc3nc(N4CCC[C@H]4CO)c4ncccc4n3)c2)cc(OC)c1OC ; COc1cc(-n2cnc(Nc3nc(N4CCC[C@@H]4C(N)=O)c4cccn4n3)c2)cc(OC)c1OC ; OC[C@@H]1CCCN1c1nc(Nc2n[nH]c3ccccc23)nc2ccccc12 ; COc1cc(-n2cnc(Nc3nc4c(c(N5CCC[C@H]5CO)n3)CN(Cc3ccccc3)CC4)c2)cc(OC)c1OC ; COc1cc(-n2cnc(Nc3nc(N4CCC[C@H]4C(N)=O)c4cc[nH]c4n3)c2)cc(OC)c1OC] 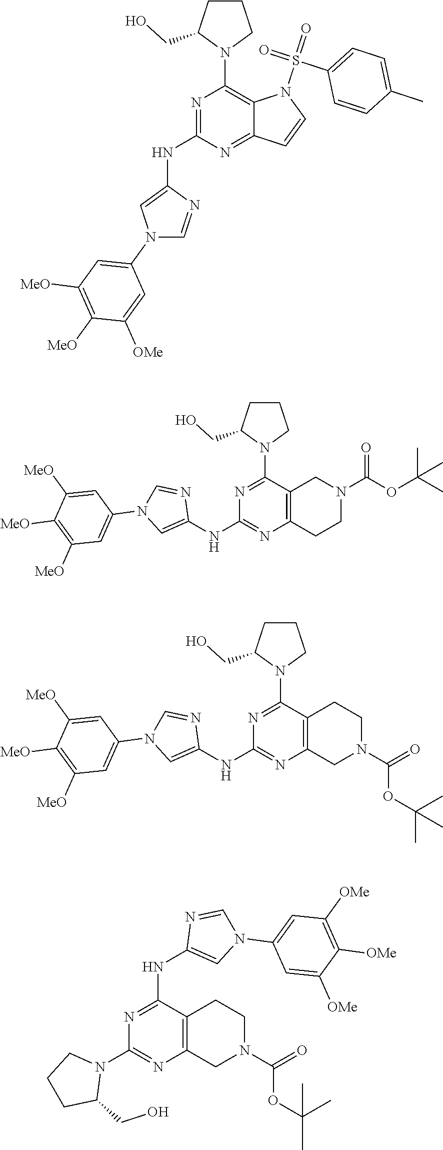 [SMILES: COc1cc(-n2cnc(Nc3nc(N4CCC[C@H]4CO)c4c(ccn4S(=O)(=O)c4ccc(C)cc4)n3)c2)cc(OC)c1OC ; COc1cc(-n2cnc(Nc3nc4c(c(N5CCC[C@H]5CO)n3)CN(C(=O)OC(C)(C)C)CC4)c2)cc(OC)c1OC ; COc1cc(-n2cnc(Nc3nc4c(c(N5CCC[C@H]5CO)n3)CCN(C(=O)OC(C)(C)C)C4)c2)cc(OC)c1OC ; COc1cc(-n2cnc(Nc3nc(N4CCC[C@H]4CO)nc4c3CCN(C(=O)OC(C)(C)C)C4)c2)cc(OC)c1OC] 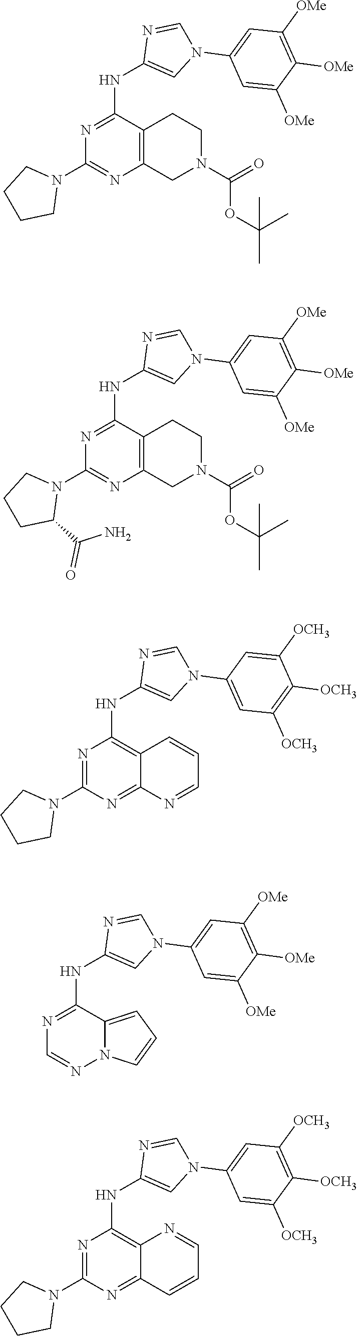 [SMILES: COc1cc(-n2cnc(Nc3nc(N4CCCC4)nc4c3CCN(C(=O)OC(C)(C)C)C4)c2)cc(OC)c1OC ; COc1cc(-n2cnc(Nc3nc(N4CCC[C@H]4C(N)=O)nc4c3CCN(C(=O)OC(C)(C)C)C4)c2)cc(OC)c1OC ; COc1cc(-n2cnc(Nc3nc(N4CCCC4)nc4ncccc34)c2)cc(OC)c1OC ; COc1cc(-n2cnc(Nc3ncnn4cccc34)c2)cc(OC)c1OC ; COc1cc(-n2cnc(Nc3nc(N4CCCC4)nc4cccnc34)c2)cc(OC)c1OC] 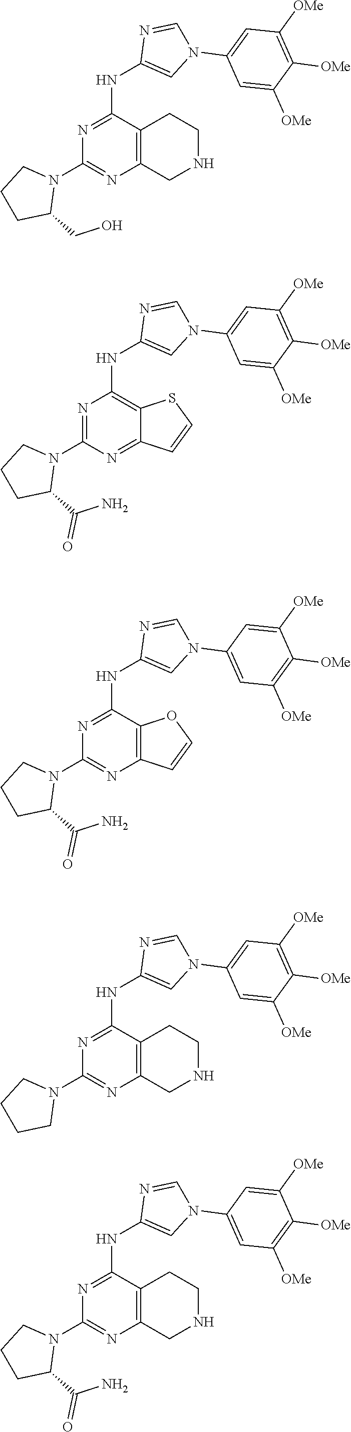 [SMILES: COc1cc(-n2cnc(Nc3nc(N4CCC[C@H]4CO)nc4c3CCNC4)c2)cc(OC)c1OC ; COc1cc(-n2cnc(Nc3nc(N4CCC[C@H]4C(N)=O)nc4ccsc34)c2)cc(OC)c1OC ; COc1cc(-n2cnc(Nc3nc(N4CCC[C@H]4C(N)=O)nc4ccoc34)c2)cc(OC)c1OC ; COc1cc(-n2cnc(Nc3nc(N4CCCC4)nc4c3CCNC4)c2)cc(OC)c1OC ; COc1cc(-n2cnc(Nc3nc(N4CCC[C@H]4C(N)=O)nc4c3CCNC4)c2)cc(OC)c1OC] 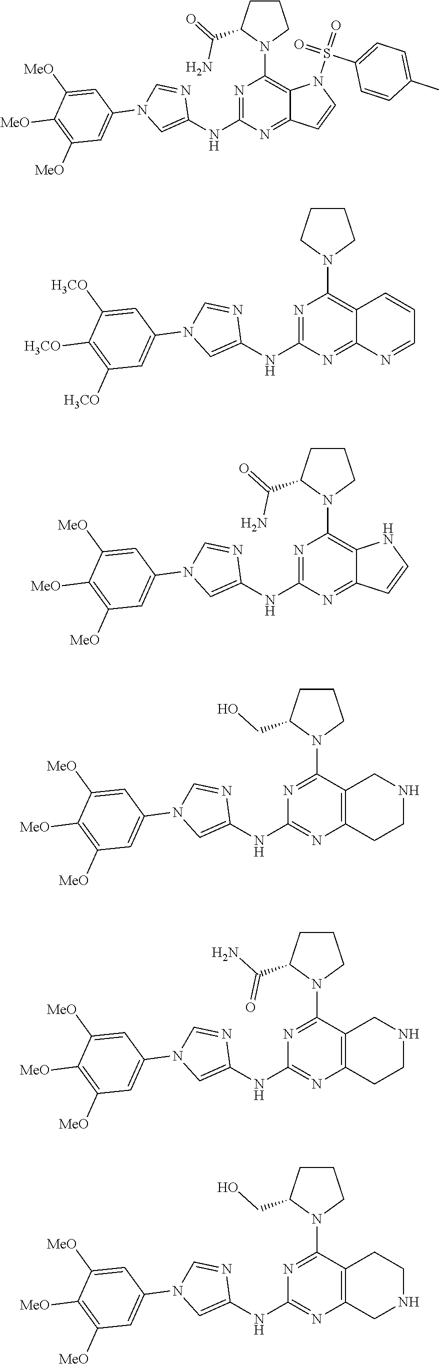 [SMILES: COc1cc(-n2cnc(Nc3nc(N4CCC[C@H]4C(N)=O)c4c(ccn4S(=O)(=O)c4ccc(C)cc4)n3)c2)cc(OC)c1OC ; COc1cc(-n2cnc(Nc3nc(N4CCCC4)c4cccnc4n3)c2)cc(OC)c1OC ; COc1cc(-n2cnc(Nc3nc(N4CCC[C@H]4C(N)=O)c4[nH]ccc4n3)c2)cc(OC)c1OC ; COc1cc(-n2cnc(Nc3nc4c(c(N5CCC[C@H]5CO)n3)CNCC4)c2)cc(OC)c1OC ; COc1cc(-n2cnc(Nc3nc4c(c(N5CCC[C@H]5C(N)=O)n3)CNCC4)c2)cc(OC)c1OC ; COc1cc(-n2cnc(Nc3nc4c(c(N5CCC[C@H]5CO)n3)CCNC4)c2)cc(OC)c1OC] 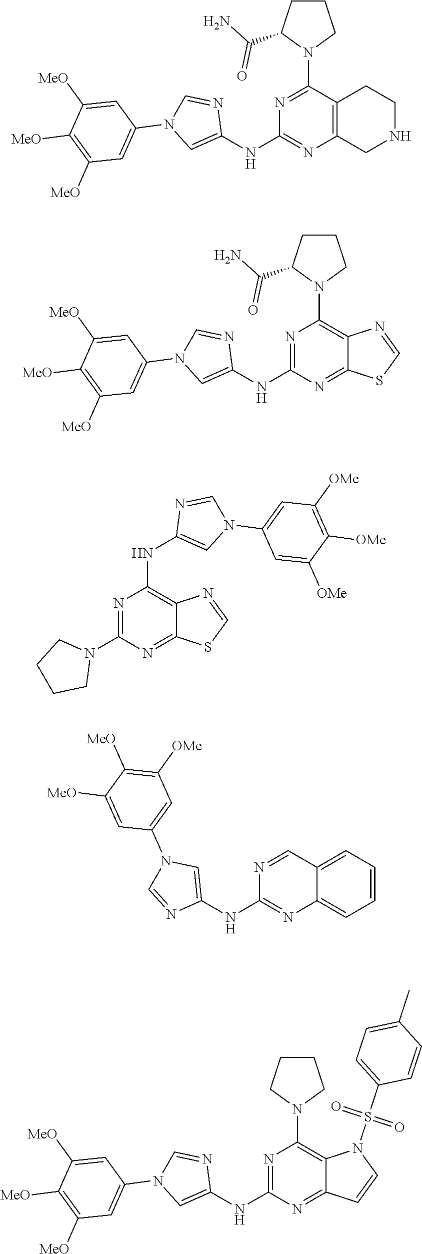 [SMILES: COc1cc(-n2cnc(Nc3nc4c(c(N5CCC[C@H]5C(N)=O)n3)CCNC4)c2)cc(OC)c1OC ; COc1cc(-n2cnc(Nc3nc(N4CCC[C@H]4C(N)=O)c4ncsc4n3)c2)cc(OC)c1OC ; COc1cc(-n2cnc(Nc3nc(N4CCCC4)nc4scnc34)c2)cc(OC)c1OC ; COc1cc(-n2cnc(Nc3ncc4ccccc4n3)c2)cc(OC)c1OC ; COc1cc(-n2cnc(Nc3nc(N4CCCC4)c4c(ccn4S(=O)(=O)c4ccc(C)cc4)n3)c2)cc(OC)c1OC] 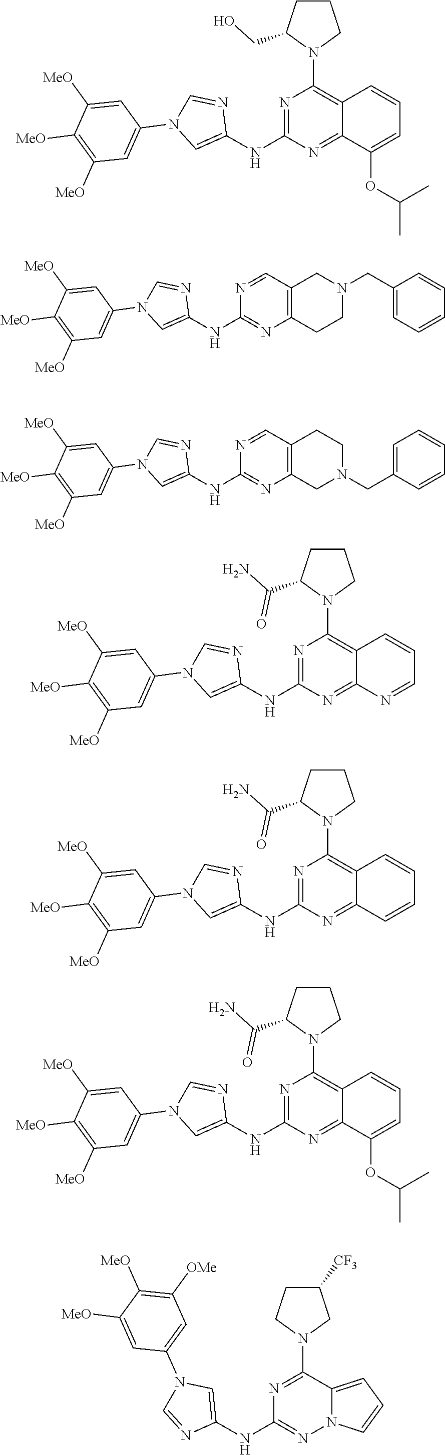 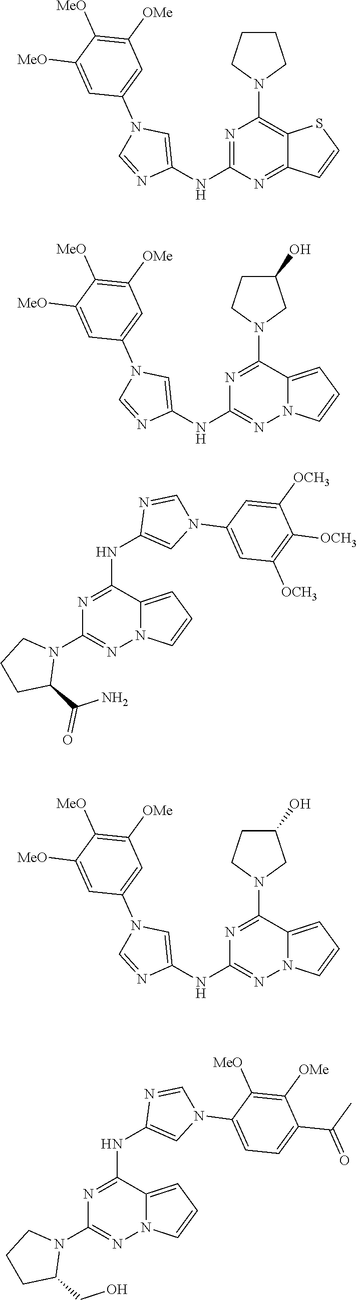 [SMILES: COc1cc(-n2cnc(Nc3nc(N4CCCC4)c4sccc4n3)c2)cc(OC)c1OC ; COc1cc(-n2cnc(Nc3nc(N4CC[C@@H](O)C4)c4cccn4n3)c2)cc(OC)c1OC ; COc1cc(-n2cnc(Nc3nc(N4CCC[C@@H]4C(N)=O)nn4cccc34)c2)cc(OC)c1OC ; COc1cc(-n2cnc(Nc3nc(N4CC[C@H](O)C4)c4cccn4n3)c2)cc(OC)c1OC ; COc1c(C(C)=O)ccc(-n2cnc(Nc3nc(N4CCC[C@H]4CO)nn4cccc34)c2)c1OC] 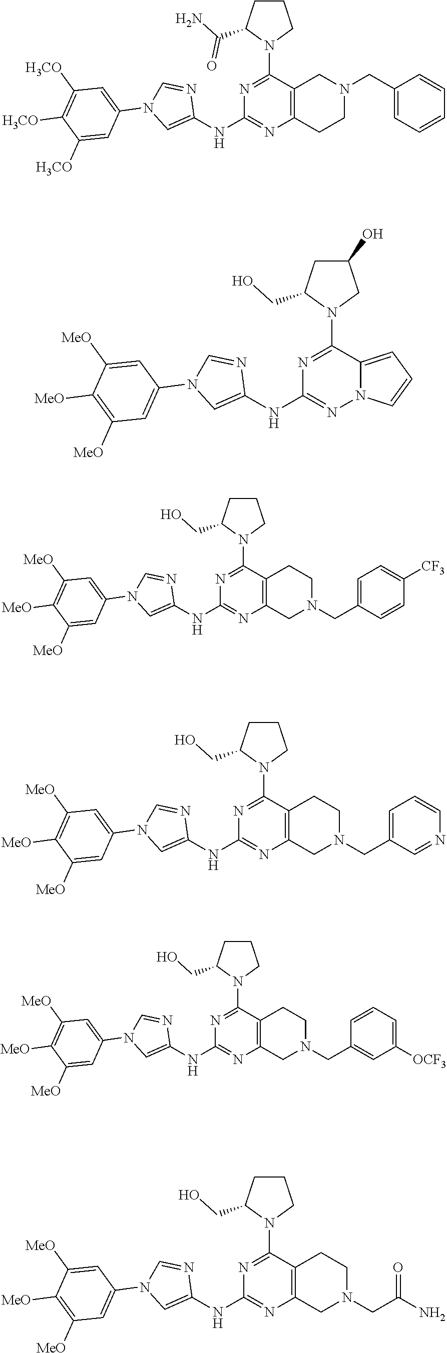 [SMILES: COc1cc(-n2cnc(Nc3nc4c(c(N5CCC[C@H]5C(N)=O)n3)CN(Cc3ccccc3)CC4)c2)cc(OC)c1OC ; COc1cc(-n2cnc(Nc3nc(N4C[C@H](O)C[C@H]4CO)c4cccn4n3)c2)cc(OC)c1OC ; COc1cc(-n2cnc(Nc3nc4c(c(N5CCC[C@H]5CO)n3)CCN(Cc3ccc(C(F)(F)F)cc3)C4)c2)cc(OC)c1OC ; COc1cc(-n2cnc(Nc3nc4c(c(N5CCC[C@H]5CO)n3)CCN(Cc3cccnc3)C4)c2)cc(OC)c1OC ; COc1cc(-n2cnc(Nc3nc4c(c(N5CCC[C@H]5CO)n3)CCN(Cc3cccc(OC(F)(F)F)c3)C4)c2)cc(OC)c1OC ; COc1cc(-n2cnc(Nc3nc4c(c(N5CCC[C@H]5CO)n3)CCN(CC(N)=O)C4)c2)cc(OC)c1OC] 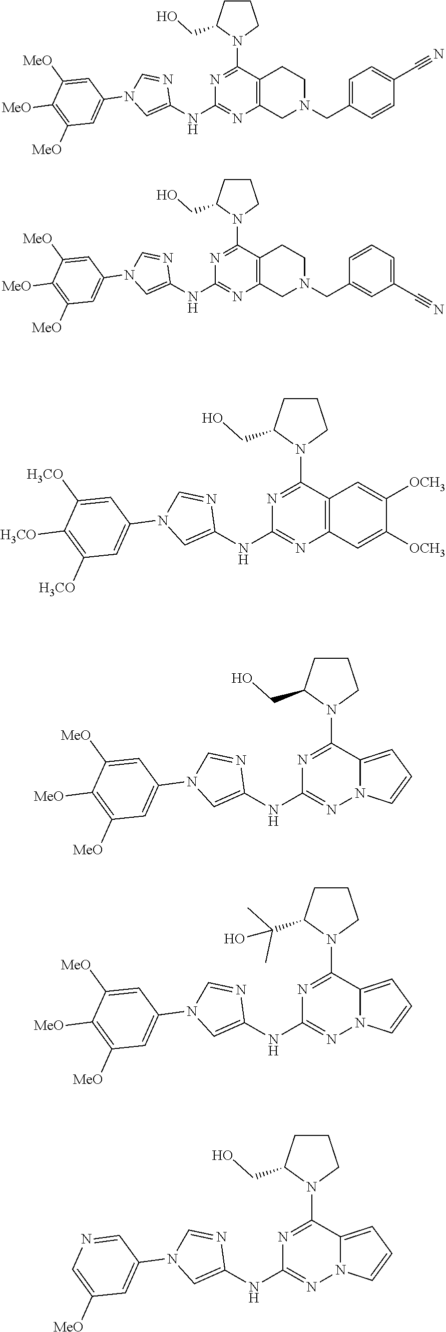 [SMILES: COc1cc(-n2cnc(Nc3nc4c(c(N5CCC[C@H]5CO)n3)CCN(Cc3ccc(C#N)cc3)C4)c2)cc(OC)c1OC ; COc1cc(-n2cnc(Nc3nc4c(c(N5CCC[C@H]5CO)n3)CCN(Cc3cccc(C#N)c3)C4)c2)cc(OC)c1OC ; COc1cc2nc(Nc3cn(-c4cc(OC)c(OC)c(OC)c4)cn3)nc(N3CCC[C@H]3CO)c2cc1OC ; COc1cc(-n2cnc(Nc3nc(N4CCC[C@@H]4CO)c4cccn4n3)c2)cc(OC)c1OC ; COc1cc(-n2cnc(Nc3nc(N4CCC[C@H]4C(C)(C)O)c4cccn4n3)c2)cc(OC)c1OC ; COc1cncc(-n2cnc(Nc3nc(N4CCC[C@H]4CO)c4cccn4n3)c2)c1] 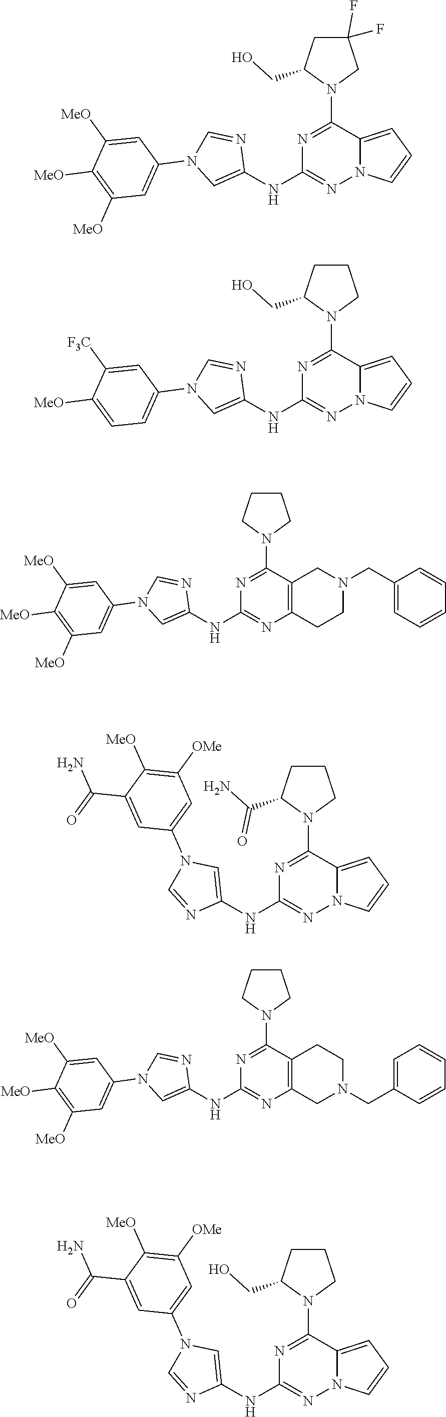 [SMILES: COc1cc(-n2cnc(Nc3nc(N4CC(F)(F)C[C@H]4CO)c4cccn4n3)c2)cc(OC)c1OC ; COc1ccc(-n2cnc(Nc3nc(N4CCC[C@H]4CO)c4cccn4n3)c2)cc1C(F)(F)F ; COc1cc(-n2cnc(Nc3nc4c(c(N5CCCC5)n3)CN(Cc3ccccc3)CC4)c2)cc(OC)c1OC ; COc1cc(-n2cnc(Nc3nc(N4CCC[C@H]4C(N)=O)c4cccn4n3)c2)cc(C(N)=O)c1OC ; COc1cc(-n2cnc(Nc3nc4c(c(N5CCCC5)n3)CCN(Cc3ccccc3)C4)c2)cc(OC)c1OC ; COc1cc(-n2cnc(Nc3nc(N4CCC[C@H]4CO)c4cccn4n3)c2)cc(C(N)=O)c1OC] 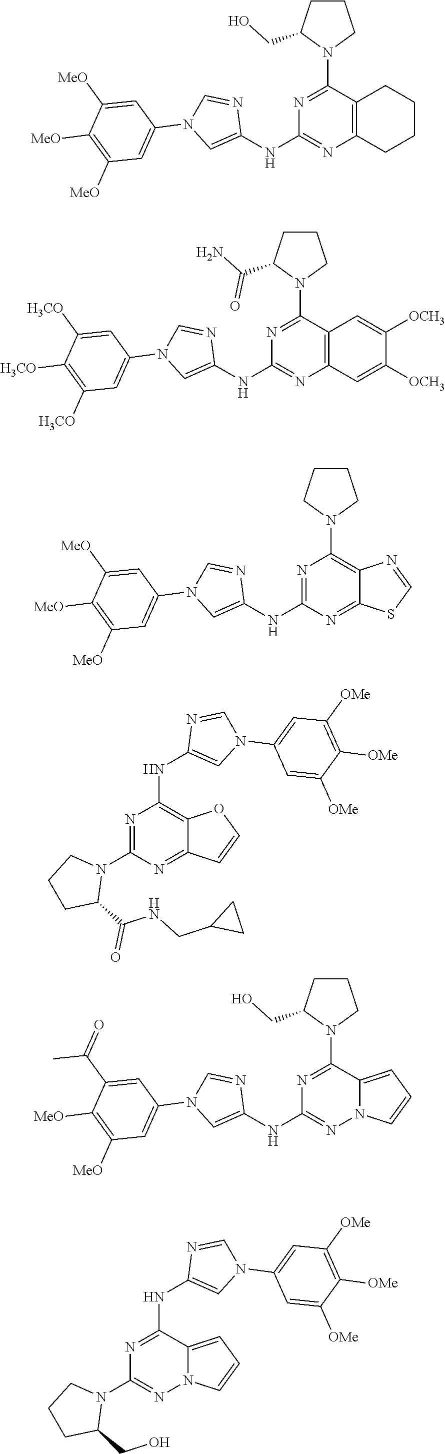 [SMILES: COc1cc(-n2cnc(Nc3nc4c(c(N5CCC[C@H]5CO)n3)CCCC4)c2)cc(OC)c1OC ; COc1cc2nc(Nc3cn(-c4cc(OC)c(OC)c(OC)c4)cn3)nc(N3CCC[C@H]3C(N)=O)c2cc1OC ; COc1cc(-n2cnc(Nc3nc(N4CCCC4)c4ncsc4n3)c2)cc(OC)c1OC ; COc1cc(-n2cnc(Nc3nc(N4CCC[C@H]4C(=O)NCC4CC4)nc4ccoc34)c2)cc(OC)c1OC ; COc1cc(-n2cnc(Nc3nc(N4CCC[C@H]4CO)c4cccn4n3)c2)cc(C(C)=O)c1OC ; COc1cc(-n2cnc(Nc3nc(N4CCC[C@@H]4CO)nn4cccc34)c2)cc(OC)c1OC] 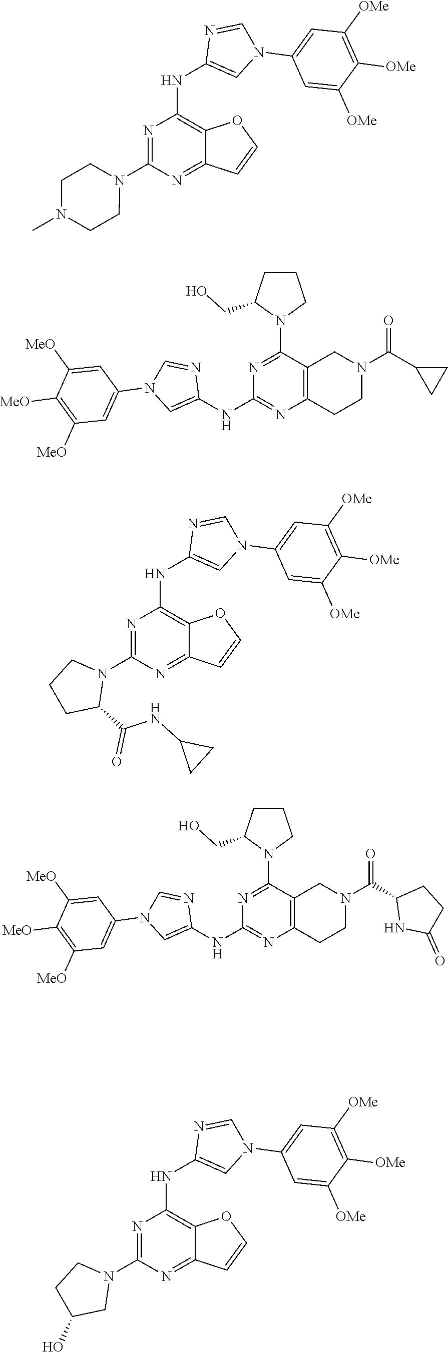 [SMILES: COc1cc(-n2cnc(Nc3nc(N4CCN(C)CC4)nc4ccoc34)c2)cc(OC)c1OC ; COc1cc(-n2cnc(Nc3nc4c(c(N5CCC[C@H]5CO)n3)CN(C(=O)C3CC3)CC4)c2)cc(OC)c1OC ; COc1cc(-n2cnc(Nc3nc(N4CCC[C@H]4C(=O)NC4CC4)nc4ccoc34)c2)cc(OC)c1OC ; COc1cc(-n2cnc(Nc3nc4c(c(N5CCC[C@H]5CO)n3)CN(C(=O)[C@@H]3CCC(=O)N3)CC4)c2)cc(OC)c1OC ; COc1cc(-n2cnc(Nc3nc(N4CC[C@@H](O)C4)nc4ccoc34)c2)cc(OC)c1OC] 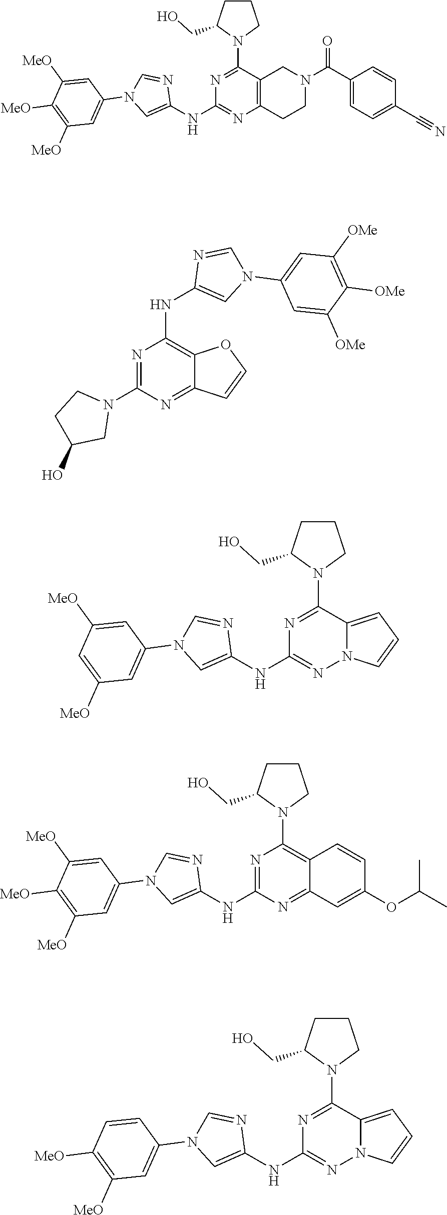 [SMILES: COc1cc(-n2cnc(Nc3nc4c(c(N5CCC[C@H]5CO)n3)CN(C(=O)c3ccc(C#N)cc3)CC4)c2)cc(OC)c1OC ; COc1cc(-n2cnc(Nc3nc(N4CC[C@H](O)C4)nc4ccoc34)c2)cc(OC)c1OC ; COc1cc(OC)cc(-n2cnc(Nc3nc(N4CCC[C@H]4CO)c4cccn4n3)c2)c1 ; COc1cc(-n2cnc(Nc3nc(N4CCC[C@H]4CO)c4ccc(OC(C)C)cc4n3)c2)cc(OC)c1OC ; COc1ccc(-n2cnc(Nc3nc(N4CCC[C@H]4CO)c4cccn4n3)c2)cc1OC] 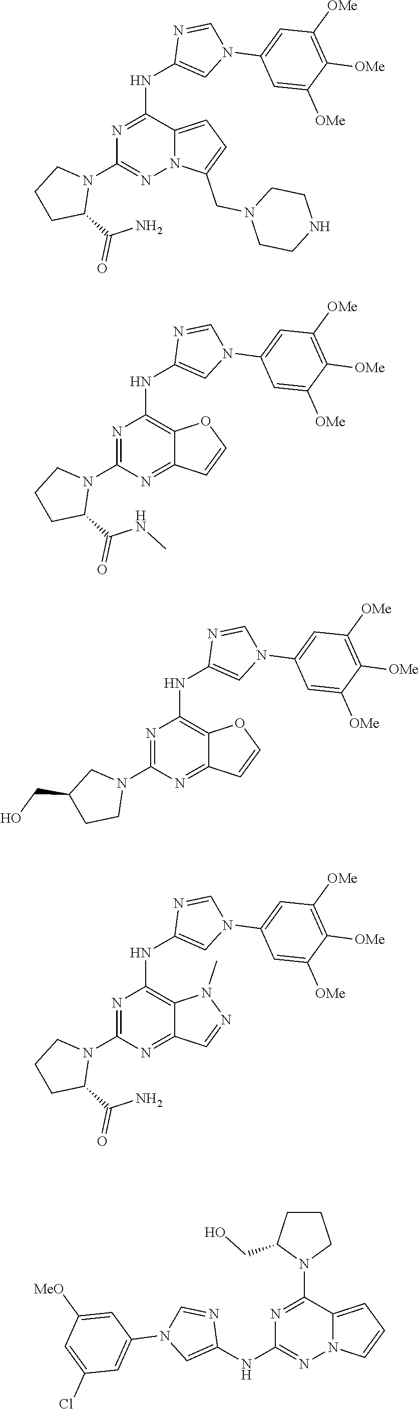 [SMILES: COc1cc(-n2cnc(Nc3nc(N4CCC[C@H]4C(N)=O)nn4c(CN5CCNCC5)ccc34)c2)cc(OC)c1OC ; CNC(=O)[C@@H]1CCCN1c1nc(Nc2cn(-c3cc(OC)c(OC)c(OC)c3)cn2)c2occc2n1 ; COc1cc(-n2cnc(Nc3nc(N4CC[C@@H](CO)C4)nc4ccoc34)c2)cc(OC)c1OC ; COc1cc(-n2cnc(Nc3nc(N4CCC[C@H]4C(N)=O)nc4cnn(C)c34)c2)cc(OC)c1OC ; COc1cc(Cl)cc(-n2cnc(Nc3nc(N4CCC[C@H]4CO)c4cccn4n3)c2)c1] 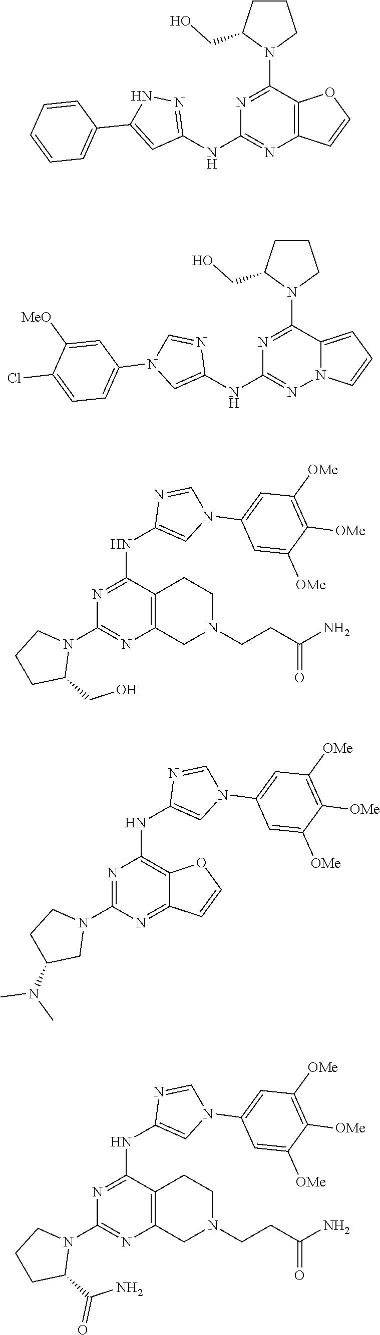 [SMILES: OC[C@@H]1CCCN1c1nc(Nc2cc(-c3ccccc3)[nH]n2)nc2ccoc12 ; COc1cc(-n2cnc(Nc3nc(N4CCC[C@H]4CO)c4cccn4n3)c2)ccc1Cl ; COc1cc(-n2cnc(Nc3nc(N4CCC[C@H]4CO)nc4c3CCN(CCC(N)=O)C4)c2)cc(OC)c1OC ; COc1cc(-n2cnc(Nc3nc(N4CC[C@@H](N(C)C)C4)nc4ccoc34)c2)cc(OC)c1OC ; COc1cc(-n2cnc(Nc3nc(N4CCC[C@H]4C(N)=O)nc4c3CCN(CCC(N)=O)C4)c2)cc(OC)c1OC] 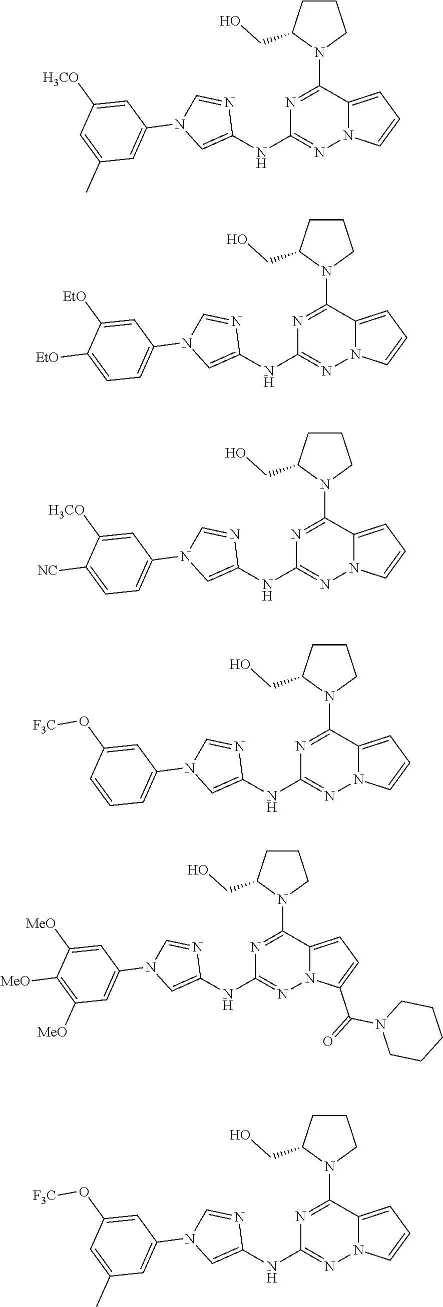 [SMILES: COc1cc(C)cc(-n2cnc(Nc3nc(N4CCC[C@H]4CO)c4cccn4n3)c2)c1 ; CCOc1ccc(-n2cnc(Nc3nc(N4CCC[C@H]4CO)c4cccn4n3)c2)cc1OCC ; COc1cc(-n2cnc(Nc3nc(N4CCC[C@H]4CO)c4cccn4n3)c2)ccc1C#N ; OC[C@@H]1CCCN1c1nc(Nc2cn(-c3cccc(OC(F)(F)F)c3)cn2)nn2cccc12 ; COc1cc(-n2cnc(Nc3nc(N4CCC[C@H]4CO)c4ccc(C(=O)N5CCCCC5)n4n3)c2)cc(OC)c1OC ; Cc1cc(OC(F)(F)F)cc(-n2cnc(Nc3nc(N4CCC[C@H]4CO)c4cccn4n3)c2)c1] 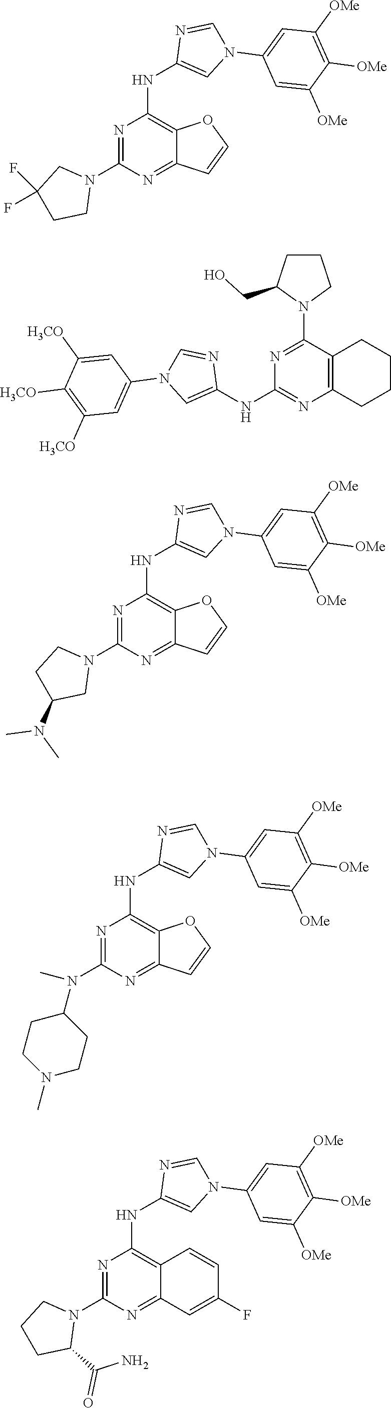 [SMILES: COc1cc(-n2cnc(Nc3nc(N4CCC(F)(F)C4)nc4ccoc34)c2)cc(OC)c1OC ; COc1cc(-n2cnc(Nc3nc4c(c(N5CCC[C@@H]5CO)n3)CCCC4)c2)cc(OC)c1OC ; COc1cc(-n2cnc(Nc3nc(N4CC[C@H](N(C)C)C4)nc4ccoc34)c2)cc(OC)c1OC ; COc1cc(-n2cnc(Nc3nc(N(C)C4CCN(C)CC4)nc4ccoc34)c2)cc(OC)c1OC ; COc1cc(-n2cnc(Nc3nc(N4CCC[C@H]4C(N)=O)nc4cc(F)ccc34)c2)cc(OC)c1OC] 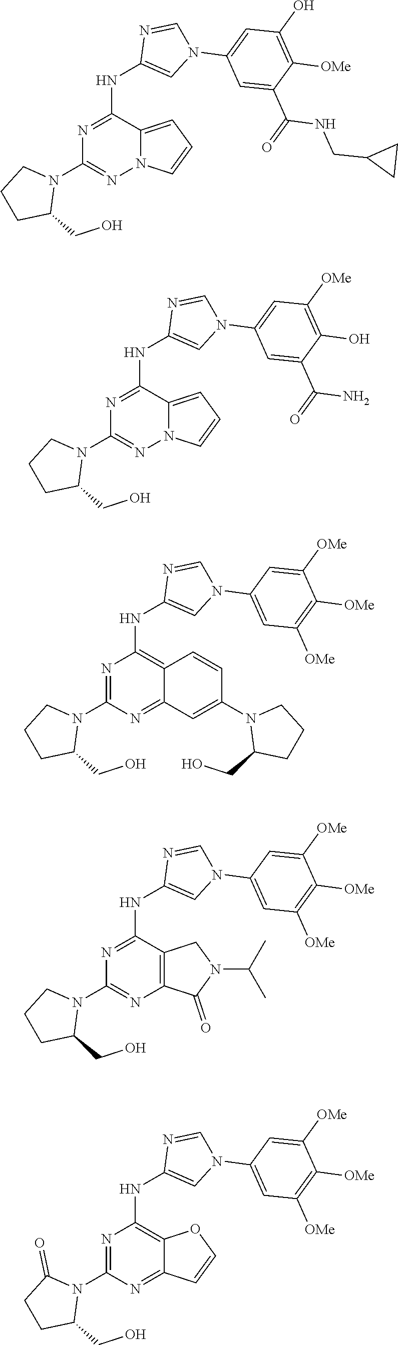 [SMILES: COc1c(O)cc(-n2cnc(Nc3nc(N4CCC[C@H]4CO)nn4cccc34)c2)cc1C(=O)NCC1CC1 ; COc1cc(-n2cnc(Nc3nc(N4CCC[C@H]4CO)nn4cccc34)c2)cc(C(N)=O)c1O ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4CO)nc4c3CN(C(C)C)C4=O)c2)cc(OC)c1OC ; COc1cc(-n2cnc(Nc3nc(N4C(=O)CC[C@H]4CO)nc4ccoc34)c2)cc(OC)c1OC] 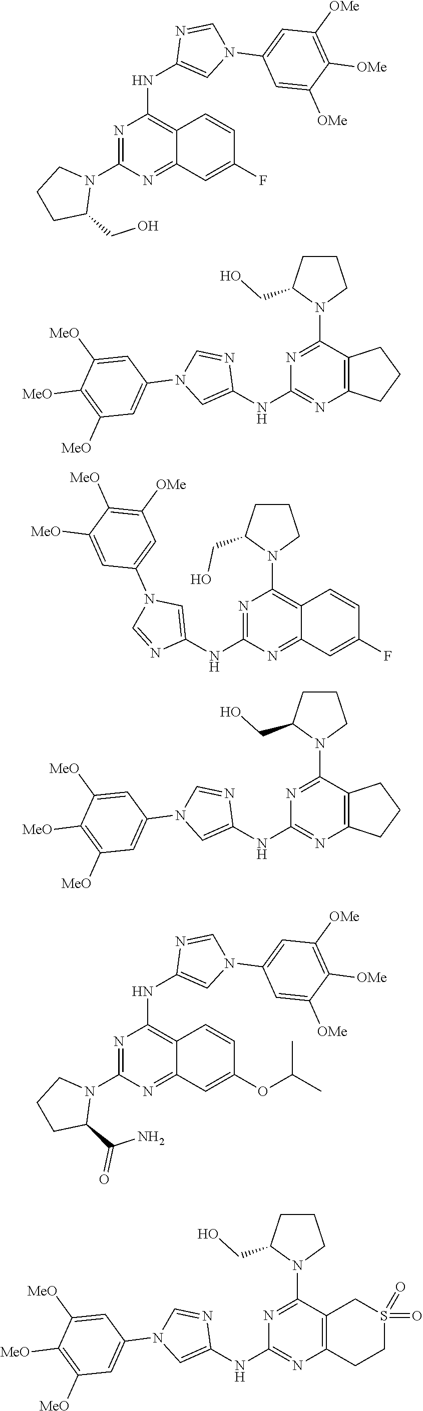 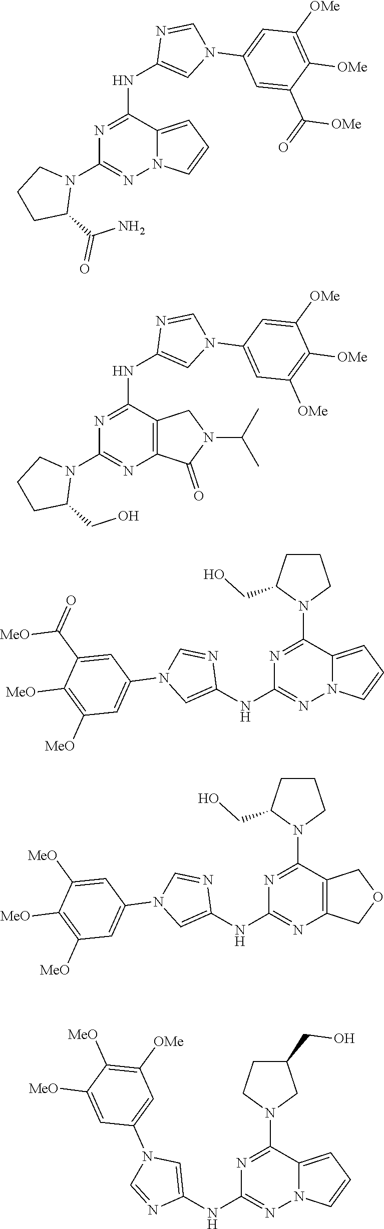 [SMILES: COC(=O)c1cc(-n2cnc(Nc3nc(N4CCC[C@H]4C(N)=O)nn4cccc34)c2)cc(OC)c1OC ; COc1cc(-n2cnc(Nc3nc(N4CCC[C@H]4CO)nc4c3CN(C(C)C)C4=O)c2)cc(OC)c1OC ; COC(=O)c1cc(-n2cnc(Nc3nc(N4CCC[C@H]4CO)c4cccn4n3)c2)cc(OC)c1OC ; COc1cc(-n2cnc(Nc3nc4c(c(N5CCC[C@H]5CO)n3)COC4)c2)cc(OC)c1OC ; COc1cc(-n2cnc(Nc3nc(N4CC[C@@H](CO)C4)c4cccn4n3)c2)cc(OC)c1OC] 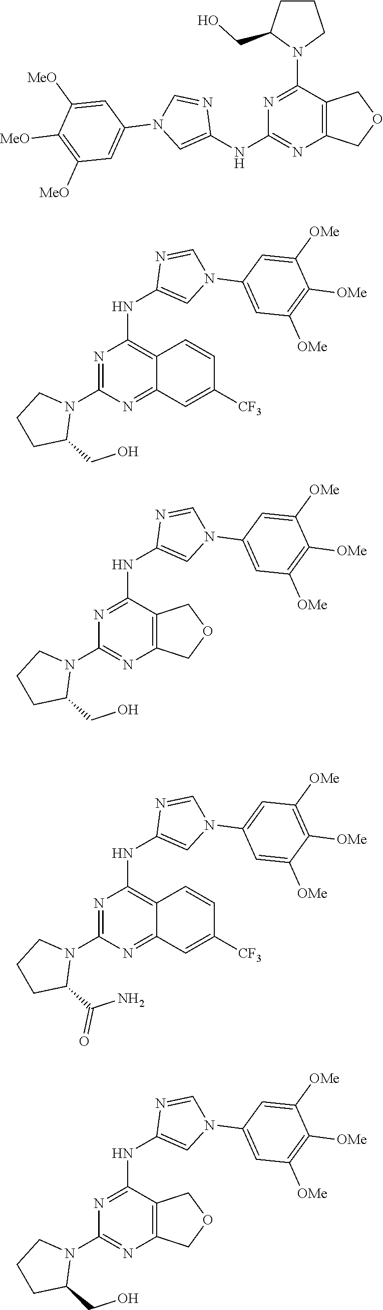 [SMILES: COc1cc(-n2cnc(Nc3nc4c(c(N5CCC[C@@H]5CO)n3)COC4)c2)cc(OC)c1OC ; COc1cc(-n2cnc(Nc3nc(N4CCC[C@H]4CO)nc4cc(C(F)(F)F)ccc34)c2)cc(OC)c1OC ; COc1cc(-n2cnc(Nc3nc(N4CCC[C@H]4CO)nc4c3COC4)c2)cc(OC)c1OC ; COc1cc(-n2cnc(Nc3nc(N4CCC[C@H]4C(N)=O)nc4cc(C(F)(F)F)ccc34)c2)cc(OC)c1OC ; COc1cc(-n2cnc(Nc3nc(N4CCC[C@@H]4CO)nc4c3COC4)c2)cc(OC)c1OC] 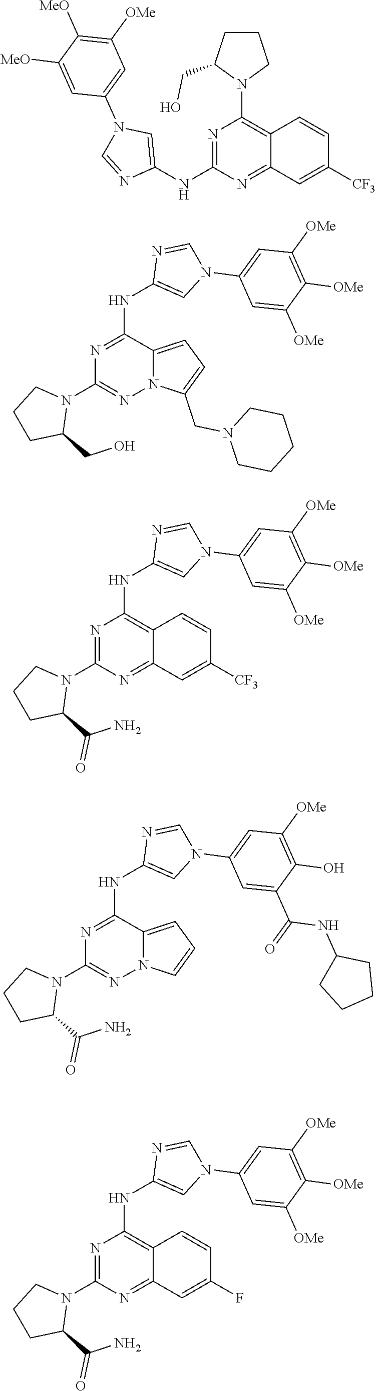 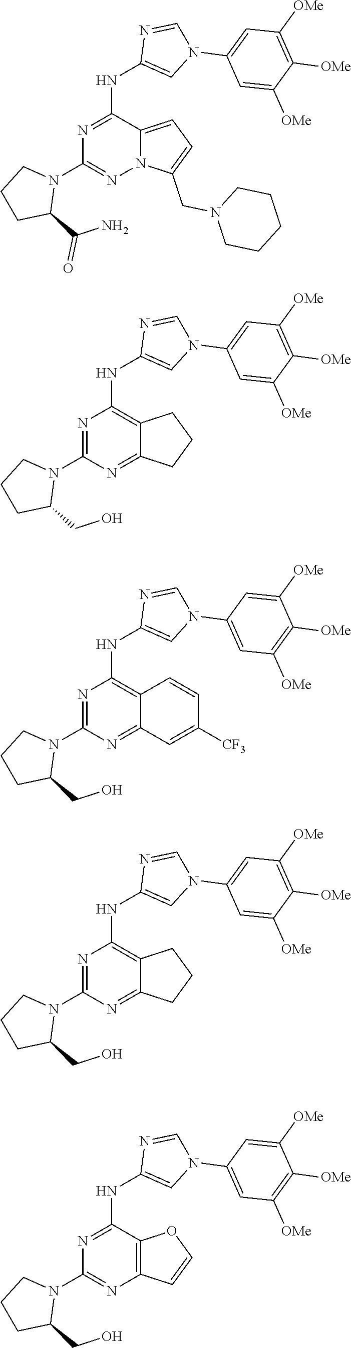 [SMILES: COc1cc(-n2cnc(Nc3nc(N4CCC[C@@H]4C(N)=O)nn4c(CN5CCCCC5)ccc34)c2)cc(OC)c1OC ; COc1cc(-n2cnc(Nc3nc(N4CCC[C@H]4CO)nc4c3CCC4)c2)cc(OC)c1OC ; COc1cc(-n2cnc(Nc3nc(N4CCC[C@@H]4CO)nc4cc(C(F)(F)F)ccc34)c2)cc(OC)c1OC ; COc1cc(-n2cnc(Nc3nc(N4CCC[C@@H]4CO)nc4c3CCC4)c2)cc(OC)c1OC ; COc1cc(-n2cnc(Nc3nc(N4CCC[C@@H]4CO)nc4ccoc34)c2)cc(OC)c1OC] 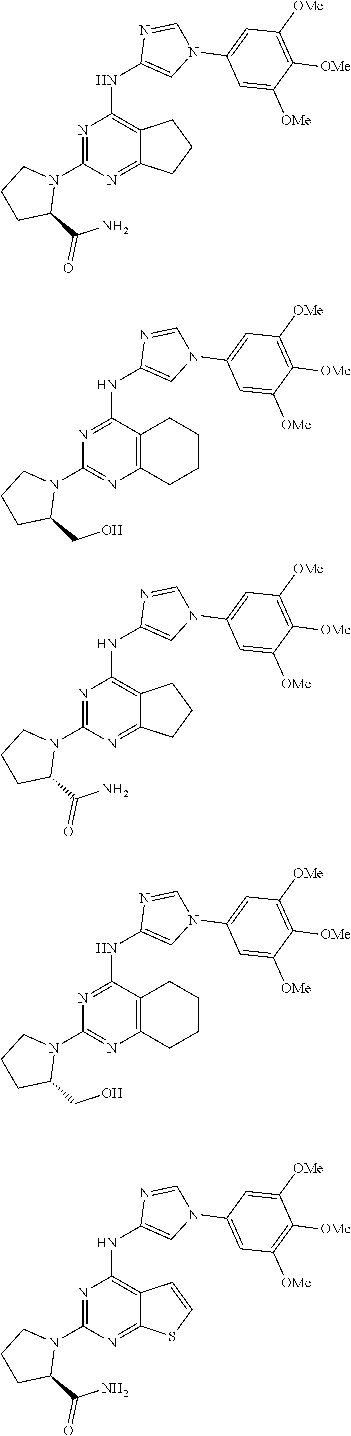 [SMILES: COc1cc(-n2cnc(Nc3nc(N4CCC[C@@H]4C(N)=O)nc4c3CCC4)c2)cc(OC)c1OC ; COc1cc(-n2cnc(Nc3nc(N4CCC[C@@H]4CO)nc4c3CCCC4)c2)cc(OC)c1OC ; COc1cc(-n2cnc(Nc3nc(N4CCC[C@H]4C(N)=O)nc4c3CCC4)c2)cc(OC)c1OC ; COc1cc(-n2cnc(Nc3nc(N4CCC[C@H]4CO)nc4c3CCCC4)c2)cc(OC)c1OC ; COc1cc(-n2cnc(Nc3nc(N4CCC[C@@H]4C(N)=O)nc4sccc34)c2)cc(OC)c1OC] 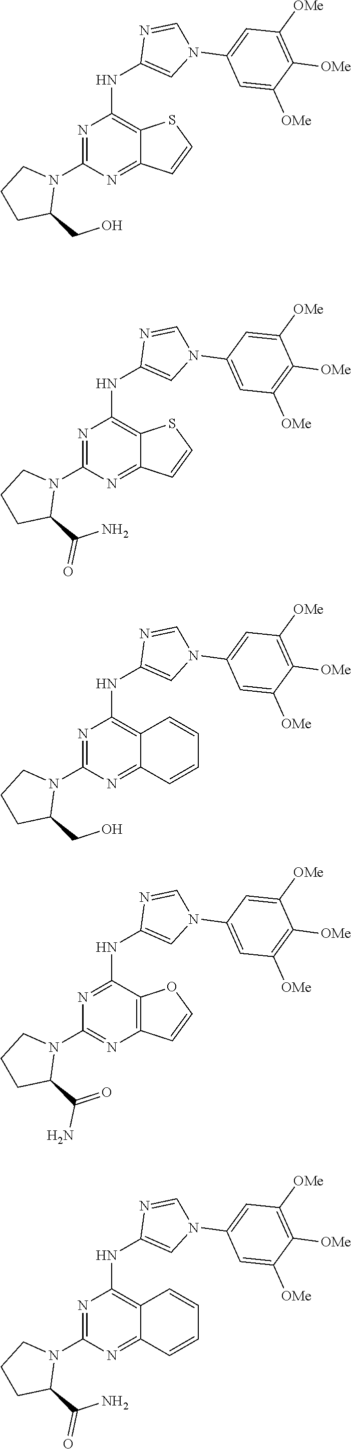 [SMILES: COc1cc(-n2cnc(Nc3nc(N4CCC[C@@H]4CO)nc4ccsc34)c2)cc(OC)c1OC ; COc1cc(-n2cnc(Nc3nc(N4CCC[C@@H]4C(N)=O)nc4ccsc34)c2)cc(OC)c1OC ; COc1cc(-n2cnc(Nc3nc(N4CCC[C@@H]4CO)nc4ccccc34)c2)cc(OC)c1OC ; COc1cc(-n2cnc(Nc3nc(N4CCC[C@@H]4C(N)=O)nc4ccoc34)c2)cc(OC)c1OC ; COc1cc(-n2cnc(Nc3nc(N4CCC[C@@H]4C(N)=O)nc4ccccc34)c2)cc(OC)c1OC] 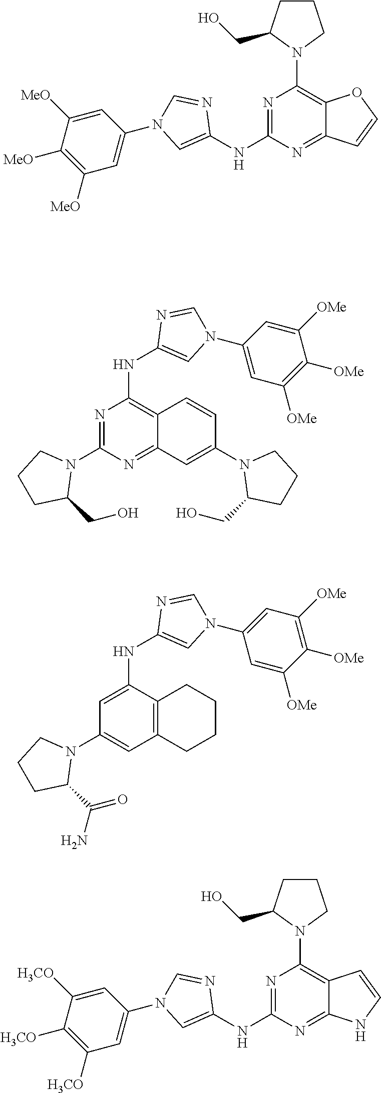 [SMILES: COc1cc(-n2cnc(Nc3nc(N4CCC[C@@H]4CO)c4occc4n3)c2)cc(OC)c1OC ; COc1cc(-n2cnc(Nc3nc(N4CCC[C@@H]4CO)nc4cc(N5CCC[C@@H]5CO)ccc34)c2)cc(OC)c1OC ; COc1cc(-n2cnc(Nc3cc(N4CCC[C@H]4C(N)=O)cc4c3CCCC4)c2)cc(OC)c1OC ; COc1cc(-n2cnc(Nc3nc(N4CCC[C@@H]4CO)c4cc[nH]c4n3)c2)cc(OC)c1OC] 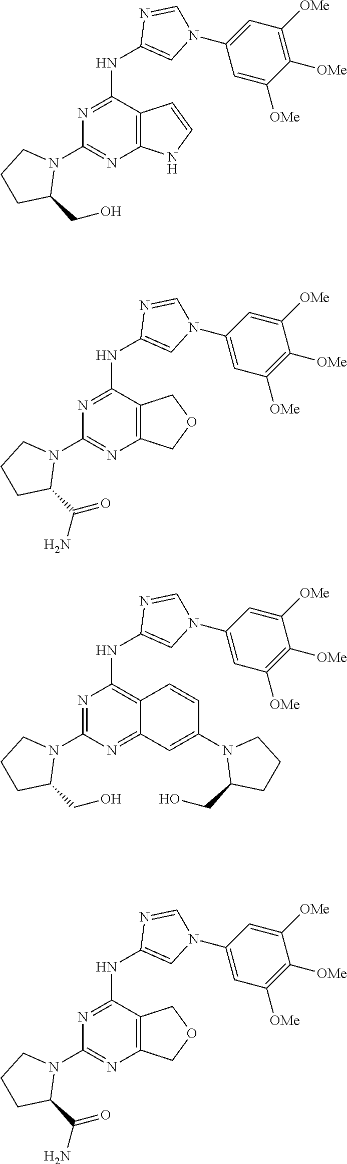 [SMILES: COc1cc(-n2cnc(Nc3nc(N4CCC[C@@H]4CO)nc4[nH]ccc34)c2)cc(OC)c1OC ; COc1cc(-n2cnc(Nc3nc(N4CCC[C@H]4C(N)=O)nc4c3COC4)c2)cc(OC)c1OC ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4C(N)=O)nc4c3COC4)c2)cc(OC)c1OC] 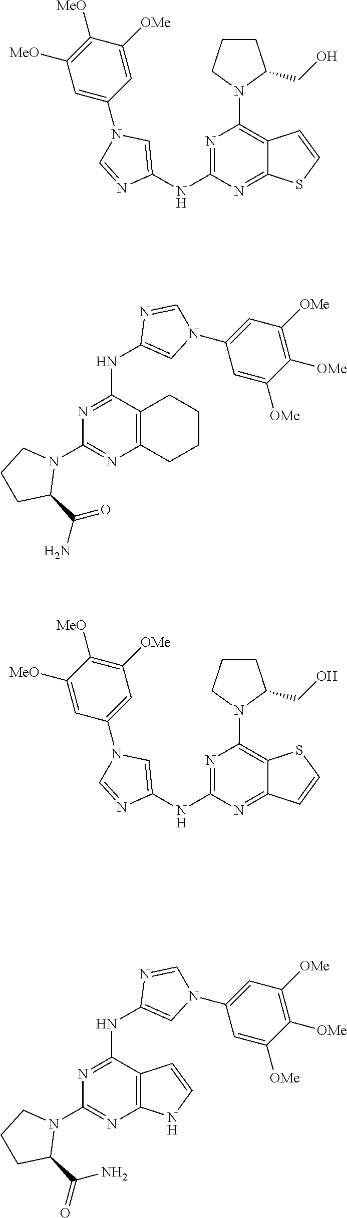 [SMILES: COc1cc(-n2cnc(Nc3nc(N4CCC[C@@H]4CO)c4ccsc4n3)c2)cc(OC)c1OC ; COc1cc(-n2cnc(Nc3nc(N4CCC[C@@H]4C(N)=O)nc4c3CCCC4)c2)cc(OC)c1OC ; COc1cc(-n2cnc(Nc3nc(N4CCC[C@@H]4CO)c4sccc4n3)c2)cc(OC)c1OC ; COc1cc(-n2cnc(Nc3nc(N4CCC[C@@H]4C(N)=O)nc4[nH]ccc34)c2)cc(OC)c1OC] 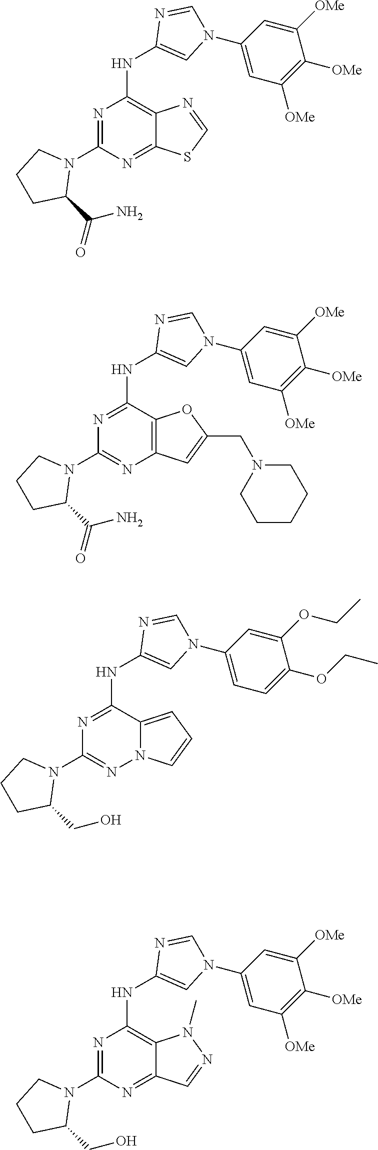 [SMILES: COc1cc(-n2cnc(Nc3nc(N4CCC[C@@H]4C(N)=O)nc4scnc34)c2)cc(OC)c1OC ; COc1cc(-n2cnc(Nc3nc(N4CCC[C@H]4C(N)=O)nc4cc(CN5CCCCC5)oc34)c2)cc(OC)c1OC ; CCOc1ccc(-n2cnc(Nc3nc(N4CCC[C@H]4CO)nn4cccc34)c2)cc1OCC ; COc1cc(-n2cnc(Nc3nc(N4CCC[C@H]4CO)nc4cnn(C)c34)c2)cc(OC)c1OC] 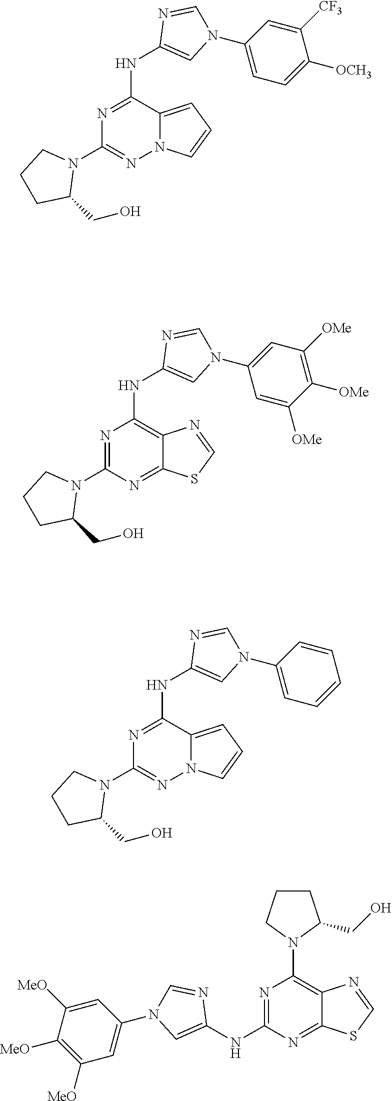 [SMILES: COc1ccc(-n2cnc(Nc3nc(N4CCC[C@H]4CO)nn4cccc34)c2)cc1C(F)(F)F ; COc1cc(-n2cnc(Nc3nc(N4CCC[C@@H]4CO)nc4scnc34)c2)cc(OC)c1OC ; OC[C@@H]1CCCN1c1nc(Nc2cn(-c3ccccc3)cn2)c2cccn2n1 ; COc1cc(-n2cnc(Nc3nc(N4CCC[C@@H]4CO)c4ncsc4n3)c2)cc(OC)c1OC] 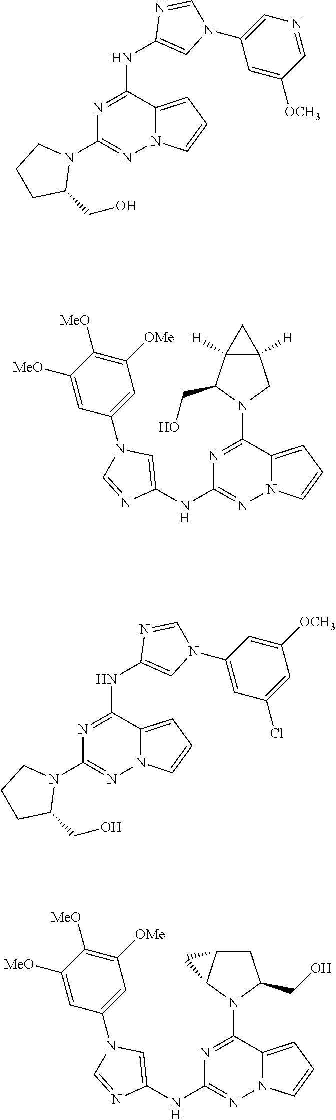 [SMILES: COc1cncc(-n2cnc(Nc3nc(N4CCC[C@H]4CO)nn4cccc34)c2)c1 ; COc1cc(-n2cnc(Nc3nc(N4C[C@@H]5C[C@@H]5[C@@H]4CO)c4cccn4n3)c2)cc(OC)c1OC ; COc1cc(Cl)cc(-n2cnc(Nc3nc(N4CCC[C@H]4CO)nn4cccc34)c2)c1 ; COc1cc(-n2cnc(Nc3nc(N4[C@H](CO)C[C@H]5C[C@H]54)c4cccn4n3)c2)cc(OC)c1OC] 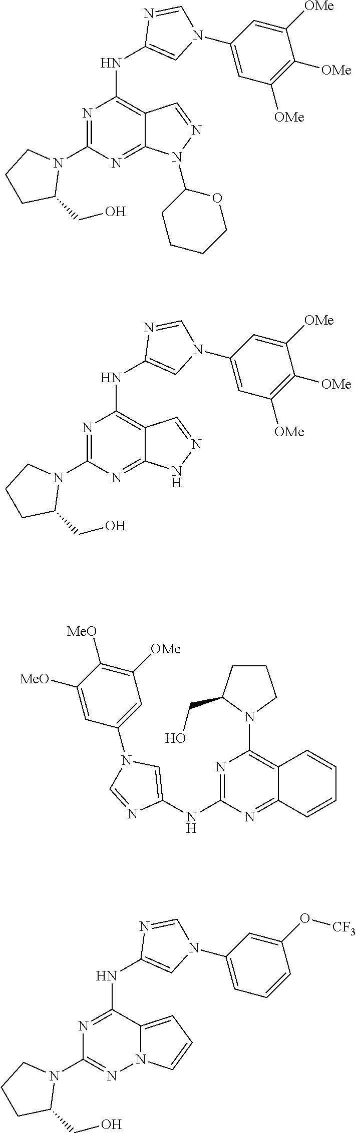 [SMILES: COc1cc(-n2cnc(Nc3nc(N4CCC[C@H]4CO)nc4c3cnn4C3CCCCO3)c2)cc(OC)c1OC ; COc1cc(-n2cnc(Nc3nc(N4CCC[C@H]4CO)nc4[nH]ncc34)c2)cc(OC)c1OC ; COc1cc(-n2cnc(Nc3nc(N4CCC[C@@H]4CO)c4ccccc4n3)c2)cc(OC)c1OC ; OC[C@@H]1CCCN1c1nc(Nc2cn(-c3cccc(OC(F)(F)F)c3)cn2)c2cccn2n1] 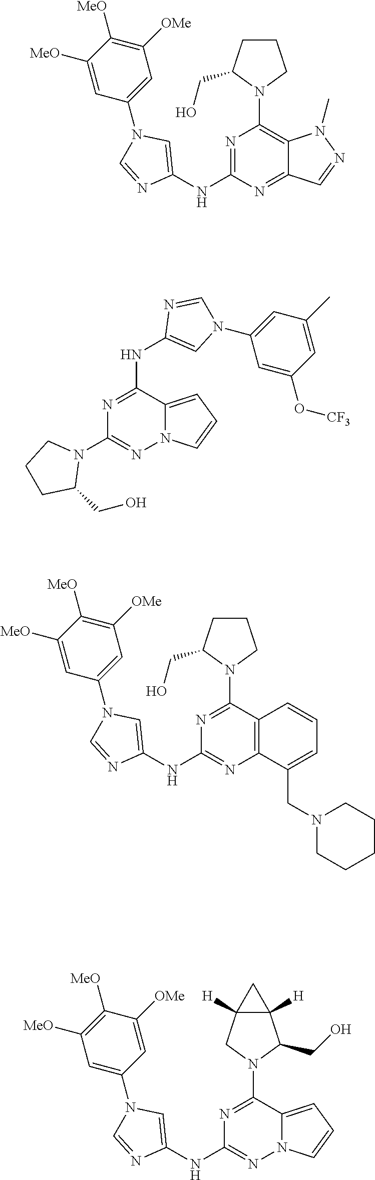 [SMILES: COc1cc(-n2cnc(Nc3nc(N4CCC[C@H]4CO)c4c(cnn4C)n3)c2)cc(OC)c1OC ; Cc1cc(OC(F)(F)F)cc(-n2cnc(Nc3nc(N4CCC[C@H]4CO)nn4cccc34)c2)c1 ; COc1cc(-n2cnc(Nc3nc(N4CCC[C@H]4CO)c4cccc(CN5CCCCC5)c4n3)c2)cc(OC)c1OC ; COc1cc(-n2cnc(Nc3nc(N4C[C@@H]5C[C@@H]5[C@H]4CO)c4cccn4n3)c2)cc(OC)c1OC] 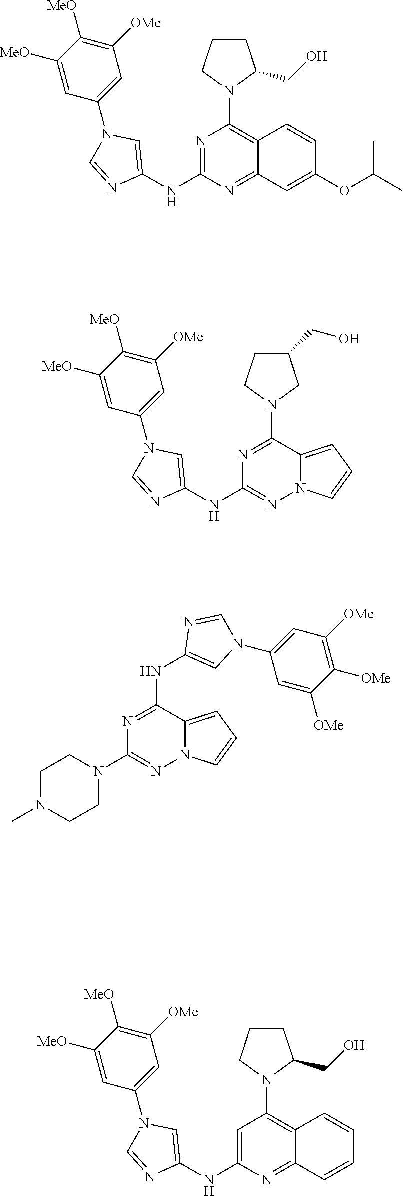 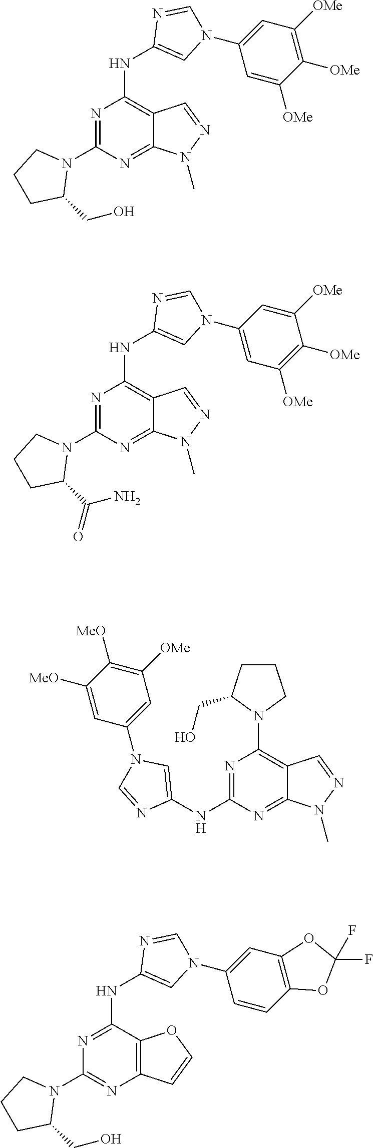 [SMILES: COc1cc(-n2cnc(Nc3nc(N4CCC[C@H]4CO)nc4c3cnn4C)c2)cc(OC)c1OC ; COc1cc(-n2cnc(Nc3nc(N4CCC[C@H]4C(N)=O)nc4c3cnn4C)c2)cc(OC)c1OC ; COc1cc(-n2cnc(Nc3nc(N4CCC[C@H]4CO)c4cnn(C)c4n3)c2)cc(OC)c1OC ; OC[C@@H]1CCCN1c1nc(Nc2cn(-c3ccc4c(c3)OC(F)(F)O4)cn2)c2occc2n1] 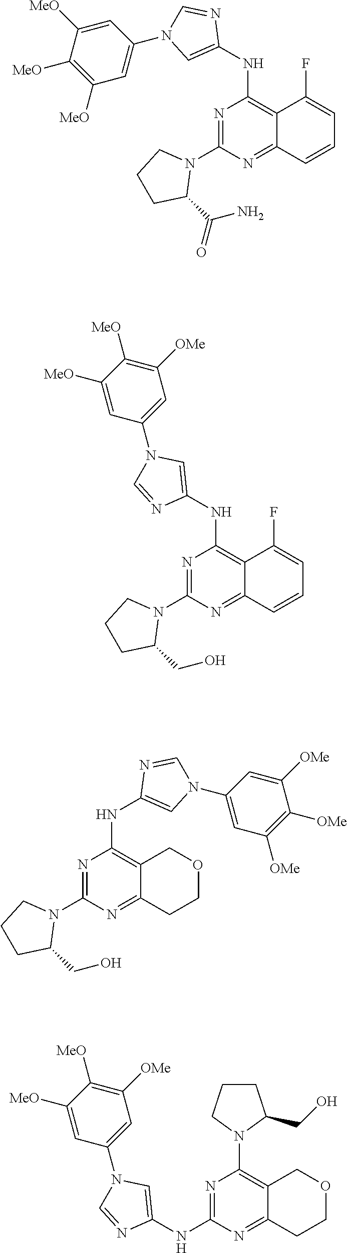 [SMILES: COc1cc(-n2cnc(Nc3nc(N4CCC[C@H]4C(N)=O)nc4cccc(F)c34)c2)cc(OC)c1OC ; COc1cc(-n2cnc(Nc3nc(N4CCC[C@H]4CO)nc4cccc(F)c34)c2)cc(OC)c1OC ; COc1cc(-n2cnc(Nc3nc(N4CCC[C@H]4CO)nc4c3COCC4)c2)cc(OC)c1OC ; COc1cc(-n2cnc(Nc3nc4c(c(N5CCC[C@H]5CO)n3)COCC4)c2)cc(OC)c1OC] 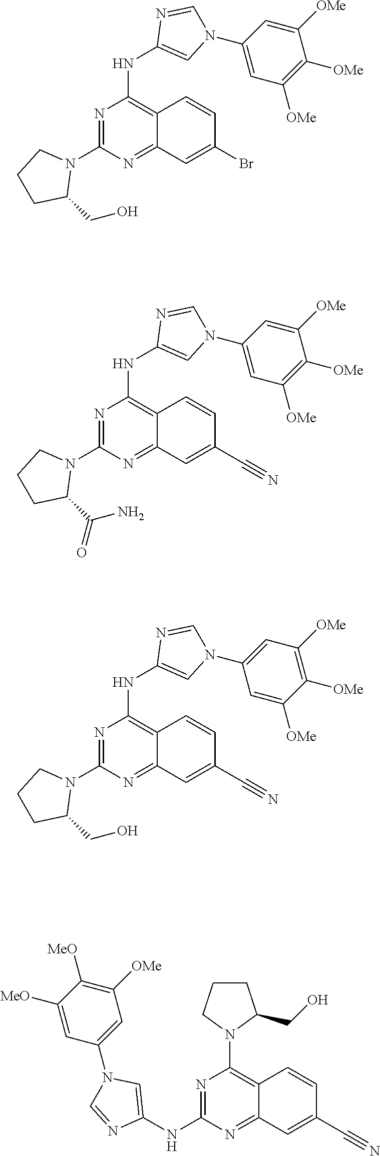 [SMILES: COc1cc(-n2cnc(Nc3nc(N4CCC[C@H]4CO)nc4cc(Br)ccc34)c2)cc(OC)c1OC ; COc1cc(-n2cnc(Nc3nc(N4CCC[C@H]4C(N)=O)nc4cc(C#N)ccc34)c2)cc(OC)c1OC ; COc1cc(-n2cnc(Nc3nc(N4CCC[C@H]4CO)nc4cc(C#N)ccc34)c2)cc(OC)c1OC ; COc1cc(-n2cnc(Nc3nc(N4CCC[C@H]4CO)c4ccc(C#N)cc4n3)c2)cc(OC)c1OC] 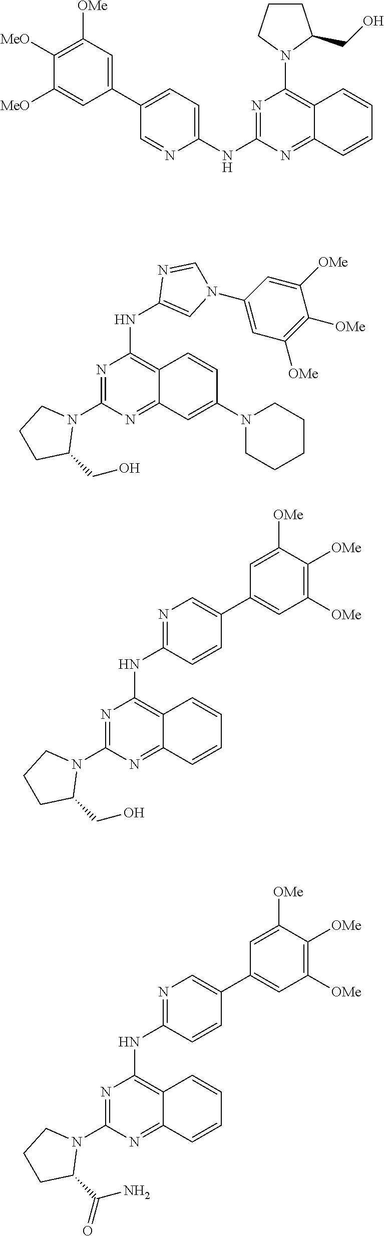 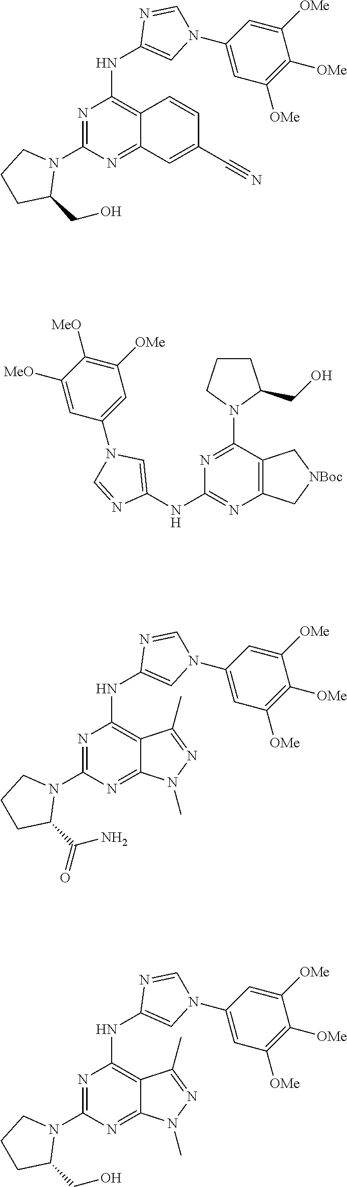 [SMILES: COc1cc(-n2cnc(Nc3nc(N4CCC[C@@H]4CO)nc4cc(C#N)ccc34)c2)cc(OC)c1OC ; COc1cc(-n2cnc(Nc3nc4c(c(N5CCC[C@H]5CO)n3)CN(C(=O)OC(C)(C)C)C4)c2)cc(OC)c1OC ; COc1cc(-n2cnc(Nc3nc(N4CCC[C@H]4C(N)=O)nc4c3c(C)nn4C)c2)cc(OC)c1OC ; COc1cc(-n2cnc(Nc3nc(N4CCC[C@H]4CO)nc4c3c(C)nn4C)c2)cc(OC)c1OC] 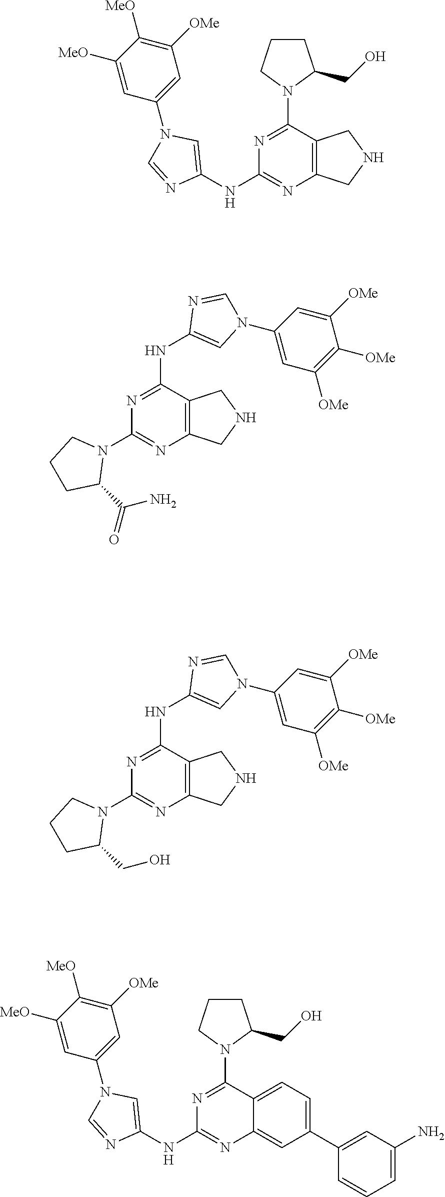 [SMILES: COc1cc(-n2cnc(Nc3nc4c(c(N5CCC[C@H]5CO)n3)CNC4)c2)cc(OC)c1OC ; COc1cc(-n2cnc(Nc3nc(N4CCC[C@H]4C(N)=O)nc4c3CNC4)c2)cc(OC)c1OC ; COc1cc(-n2cnc(Nc3nc(N4CCC[C@H]4CO)nc4c3CNC4)c2)cc(OC)c1OC ; COc1cc(-n2cnc(Nc3nc(N4CCC[C@H]4CO)c4ccc(-c5cccc(N)c5)cc4n3)c2)cc(OC)c1OC] 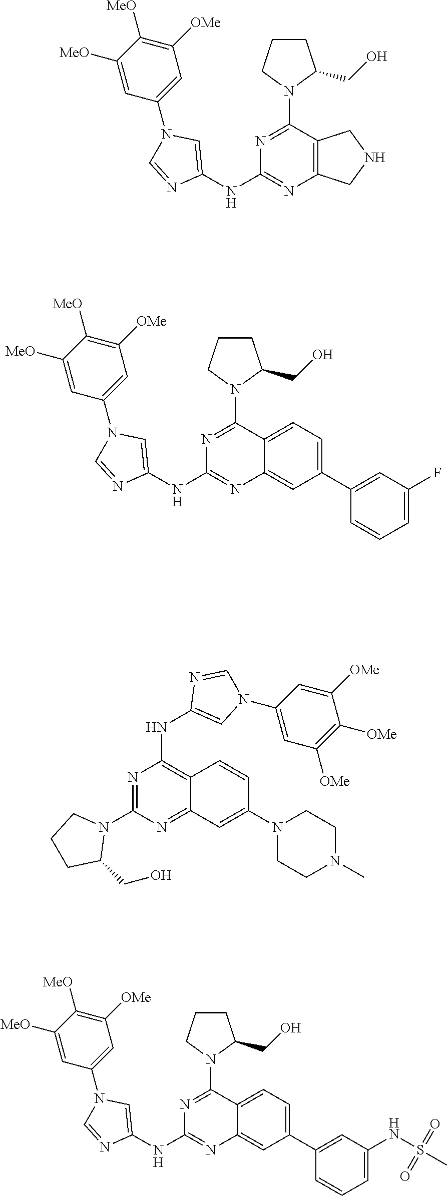 [SMILES: COc1cc(-n2cnc(Nc3nc4c(c(N5CCC[C@@H]5CO)n3)CNC4)c2)cc(OC)c1OC ; COc1cc(-n2cnc(Nc3nc(N4CCC[C@H]4CO)c4ccc(-c5cccc(F)c5)cc4n3)c2)cc(OC)c1OC ; COc1cc(-n2cnc(Nc3nc(N4CCC[C@H]4CO)nc4cc(N5CCN(C)CC5)ccc34)c2)cc(OC)c1OC ; COc1cc(-n2cnc(Nc3nc(N4CCC[C@H]4CO)c4ccc(-c5cccc(NS(C)(=O)=O)c5)cc4n3)c2)cc(OC)c1OC] 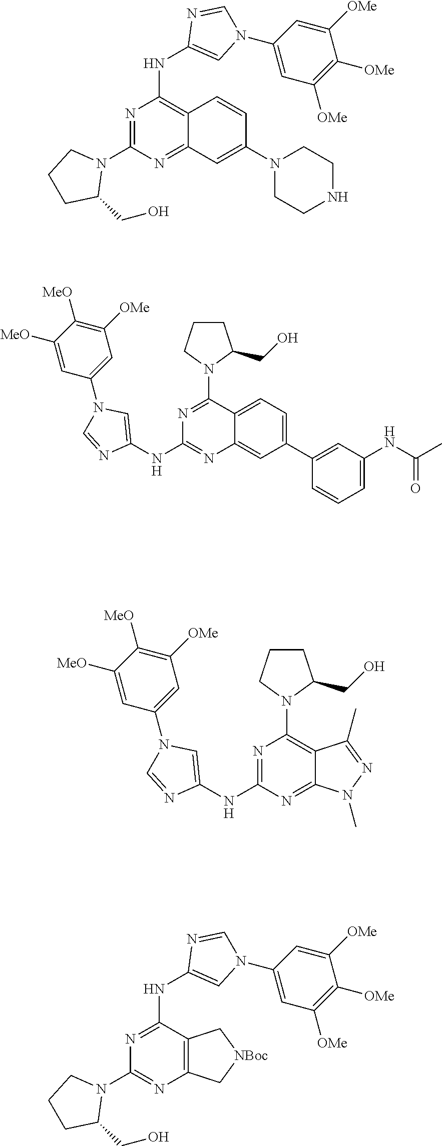 [SMILES: COc1cc(-n2cnc(Nc3nc(N4CCC[C@H]4CO)nc4cc(N5CCNCC5)ccc34)c2)cc(OC)c1OC ; COc1cc(-n2cnc(Nc3nc(N4CCC[C@H]4CO)c4ccc(-c5cccc(NC(C)=O)c5)cc4n3)c2)cc(OC)c1OC ; COc1cc(-n2cnc(Nc3nc(N4CCC[C@H]4CO)c4c(C)nn(C)c4n3)c2)cc(OC)c1OC ; COc1cc(-n2cnc(Nc3nc(N4CCC[C@H]4CO)nc4c3CN(C(=O)OC(C)(C)C)C4)c2)cc(OC)c1OC] 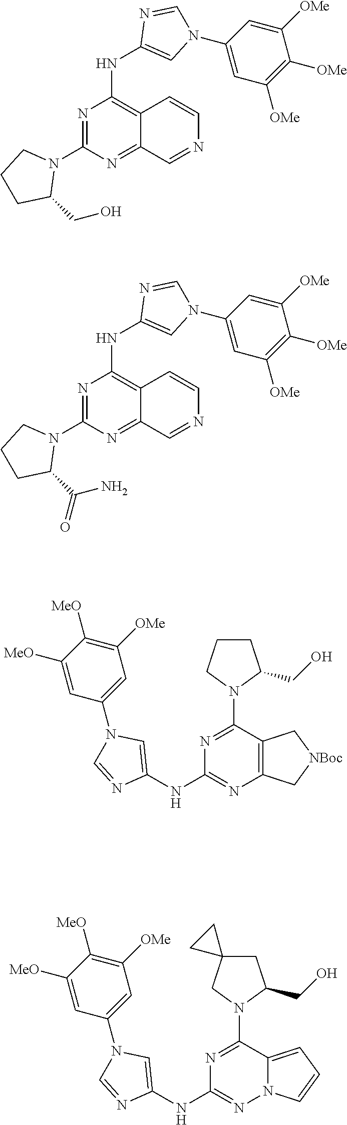 [SMILES: COc1cc(-n2cnc(Nc3nc(N4CCC[C@H]4CO)nc4cnccc34)c2)cc(OC)c1OC ; COc1cc(-n2cnc(Nc3nc(N4CCC[C@H]4C(N)=O)nc4cnccc34)c2)cc(OC)c1OC ; COc1cc(-n2cnc(Nc3nc4c(c(N5CCC[C@@H]5CO)n3)CN(C(=O)OC(C)(C)C)C4)c2)cc(OC)c1OC ; COc1cc(-n2cnc(Nc3nc(N4CC5(CC5)C[C@H]4CO)c4cccn4n3)c2)cc(OC)c1OC] 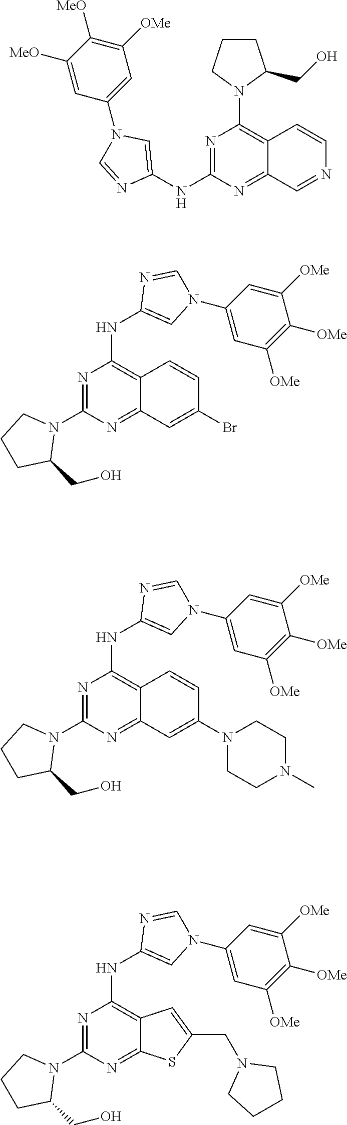 [SMILES: COc1cc(-n2cnc(Nc3nc(N4CCC[C@H]4CO)c4ccncc4n3)c2)cc(OC)c1OC ; COc1cc(-n2cnc(Nc3nc(N4CCC[C@@H]4CO)nc4cc(Br)ccc34)c2)cc(OC)c1OC ; COc1cc(-n2cnc(Nc3nc(N4CCC[C@@H]4CO)nc4cc(N5CCN(C)CC5)ccc34)c2)cc(OC)c1OC ; COc1cc(-n2cnc(Nc3nc(N4CCC[C@H]4CO)nc4sc(CN5CCCC5)cc34)c2)cc(OC)c1OC] 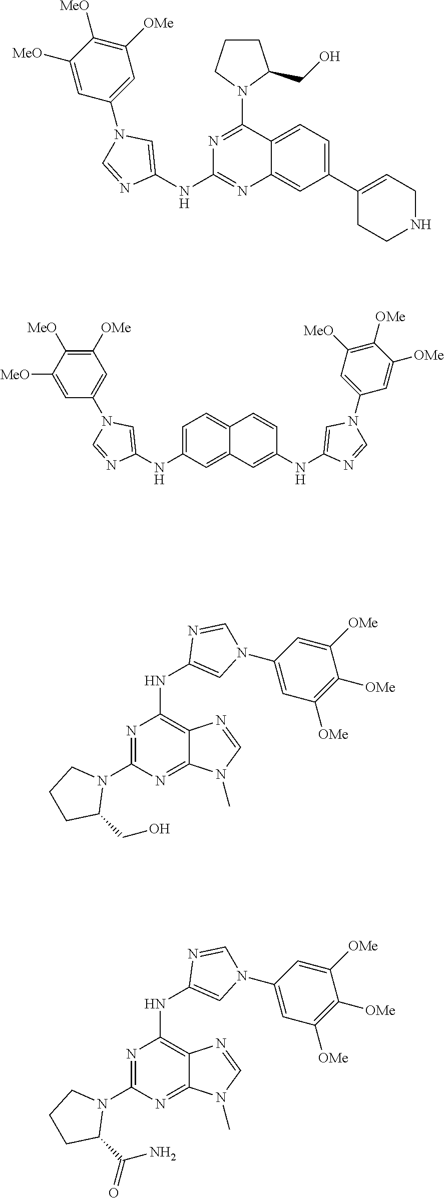 [SMILES: COc1cc(-n2cnc(Nc3nc(N4CCC[C@H]4CO)c4ccc(C5=CCNCC5)cc4n3)c2)cc(OC)c1OC ; COc1cc(-n2cnc(Nc3ccc4ccc(Nc5cn(-c6cc(OC)c(OC)c(OC)c6)cn5)cc4c3)c2)cc(OC)c1OC ; COc1cc(-n2cnc(Nc3nc(N4CCC[C@H]4CO)nc4c3ncn4C)c2)cc(OC)c1OC ; COc1cc(-n2cnc(Nc3nc(N4CCC[C@H]4C(N)=O)nc4c3ncn4C)c2)cc(OC)c1OC] 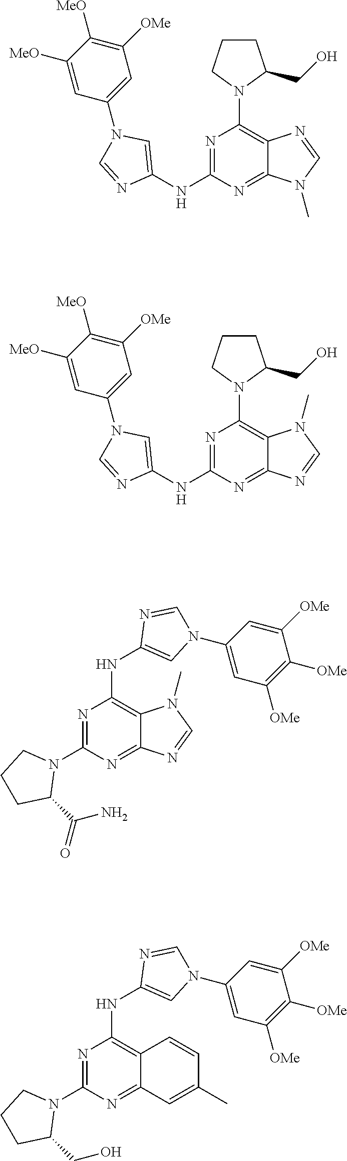 [SMILES: COc1cc(-n2cnc(Nc3nc(N4CCC[C@H]4CO)c4ncn(C)c4n3)c2)cc(OC)c1OC ; COc1cc(-n2cnc(Nc3nc(N4CCC[C@H]4CO)c4c(ncn4C)n3)c2)cc(OC)c1OC ; COc1cc(-n2cnc(Nc3nc(N4CCC[C@H]4C(N)=O)nc4ncn(C)c34)c2)cc(OC)c1OC ; COc1cc(-n2cnc(Nc3nc(N4CCC[C@H]4CO)nc4cc(C)ccc34)c2)cc(OC)c1OC] 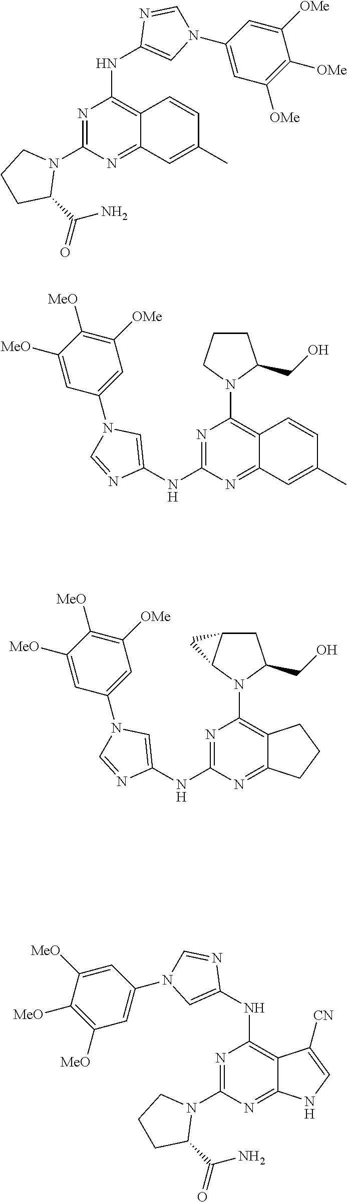 [SMILES: COc1cc(-n2cnc(Nc3nc(N4CCC[C@H]4C(N)=O)nc4cc(C)ccc34)c2)cc(OC)c1OC ; COc1cc(-n2cnc(Nc3nc(N4CCC[C@H]4CO)c4ccc(C)cc4n3)c2)cc(OC)c1OC ; COc1cc(-n2cnc(Nc3nc4c(c(N5[C@H](CO)C[C@H]6C[C@H]65)n3)CCC4)c2)cc(OC)c1OC ; COc1cc(-n2cnc(Nc3nc(N4CCC[C@H]4C(N)=O)nc4[nH]cc(C#N)c34)c2)cc(OC)c1OC] 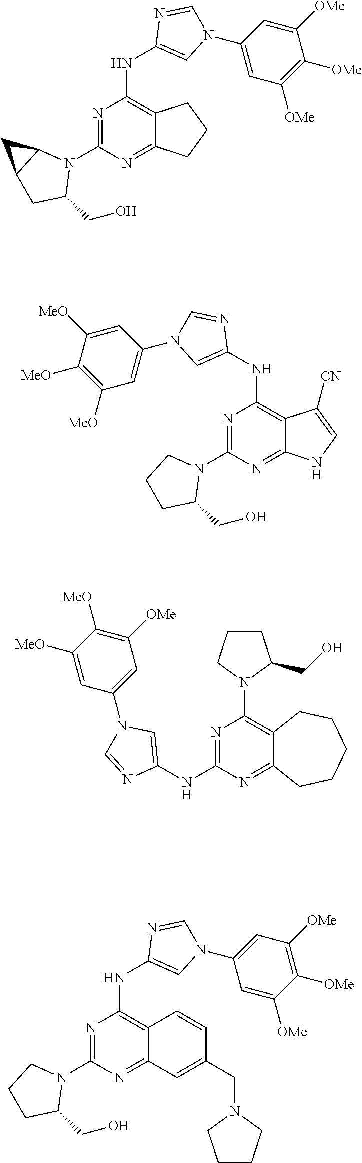 [SMILES: COc1cc(-n2cnc(Nc3nc(N4[C@H](CO)C[C@H]5C[C@H]54)nc4c3CCC4)c2)cc(OC)c1OC ; COc1cc(-n2cnc(Nc3nc(N4CCC[C@H]4CO)nc4[nH]cc(C#N)c34)c2)cc(OC)c1OC ; COc1cc(-n2cnc(Nc3nc4c(c(N5CCC[C@H]5CO)n3)CCCCC4)c2)cc(OC)c1OC ; COc1cc(-n2cnc(Nc3nc(N4CCC[C@H]4CO)nc4cc(CN5CCCC5)ccc34)c2)cc(OC)c1OC] 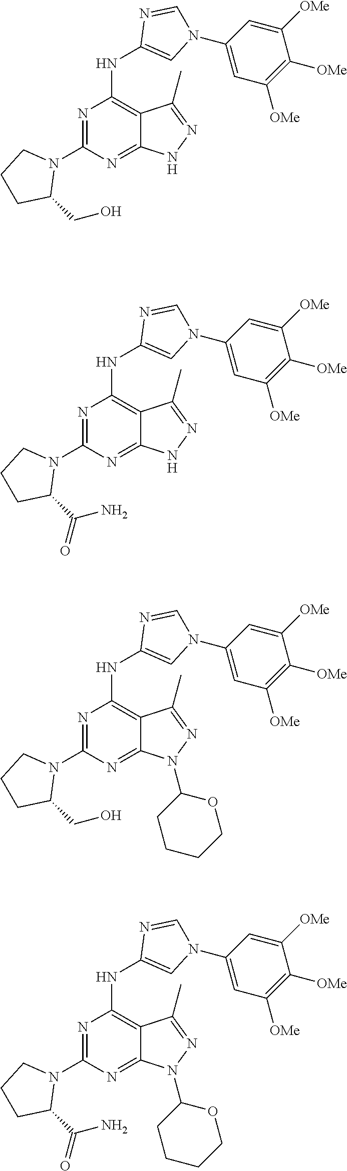 [SMILES: COc1cc(-n2cnc(Nc3nc(N4CCC[C@H]4CO)nc4[nH]nc(C)c34)c2)cc(OC)c1OC ; COc1cc(-n2cnc(Nc3nc(N4CCC[C@H]4C(N)=O)nc4[nH]nc(C)c34)c2)cc(OC)c1OC ; COc1cc(-n2cnc(Nc3nc(N4CCC[C@H]4CO)nc4c3c(C)nn4C3CCCCO3)c2)cc(OC)c1OC ; COc1cc(-n2cnc(Nc3nc(N4CCC[C@H]4C(N)=O)nc4c3c(C)nn4C3CCCCO3)c2)cc(OC)c1OC] 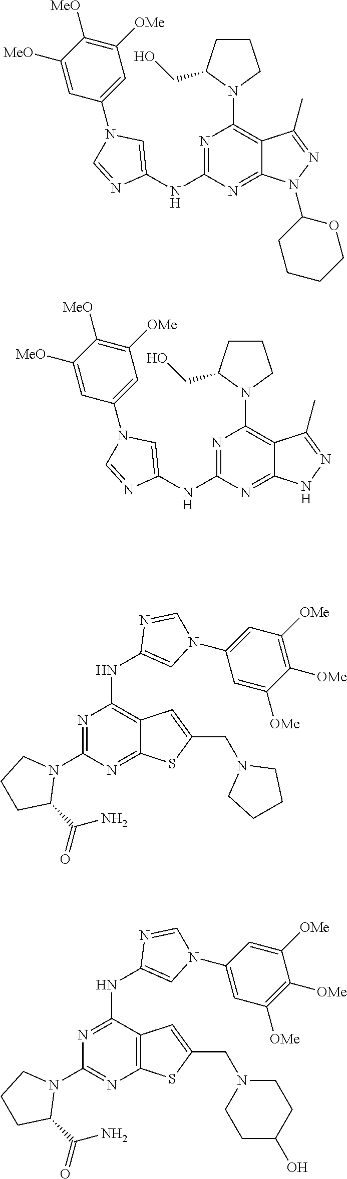 [SMILES: COc1cc(-n2cnc(Nc3nc(N4CCC[C@H]4CO)c4c(C)nn(C5CCCCO5)c4n3)c2)cc(OC)c1OC ; COc1cc(-n2cnc(Nc3nc(N4CCC[C@H]4CO)c4c(C)n[nH]c4n3)c2)cc(OC)c1OC ; COc1cc(-n2cnc(Nc3nc(N4CCC[C@H]4C(N)=O)nc4sc(CN5CCCC5)cc34)c2)cc(OC)c1OC ; COc1cc(-n2cnc(Nc3nc(N4CCC[C@H]4C(N)=O)nc4sc(CN5CCC(O)CC5)cc34)c2)cc(OC)c1OC] 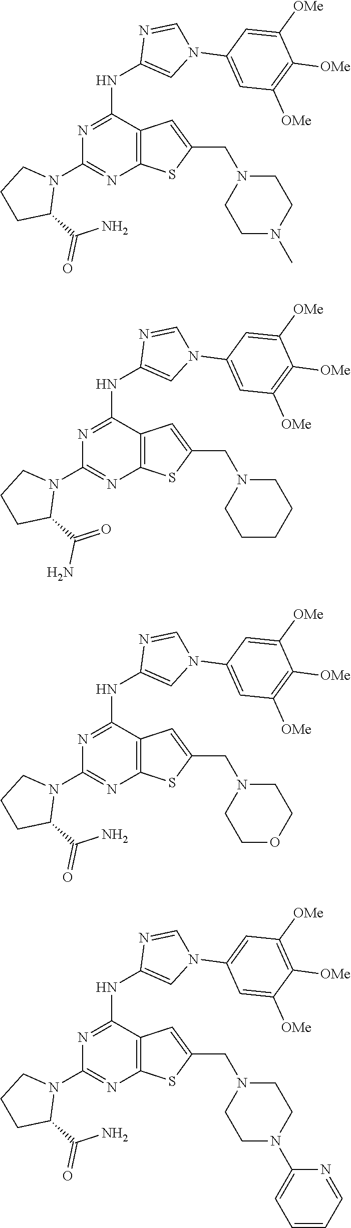 [SMILES: COc1cc(-n2cnc(Nc3nc(N4CCC[C@H]4C(N)=O)nc4sc(CN5CCN(C)CC5)cc34)c2)cc(OC)c1OC ; COc1cc(-n2cnc(Nc3nc(N4CCC[C@H]4C(N)=O)nc4sc(CN5CCCCC5)cc34)c2)cc(OC)c1OC ; COc1cc(-n2cnc(Nc3nc(N4CCC[C@H]4C(N)=O)nc4sc(CN5CCOCC5)cc34)c2)cc(OC)c1OC ; COc1cc(-n2cnc(Nc3nc(N4CCC[C@H]4C(N)=O)nc4sc(CN5CCN(c6ccccn6)CC5)cc34)c2)cc(OC)c1OC] 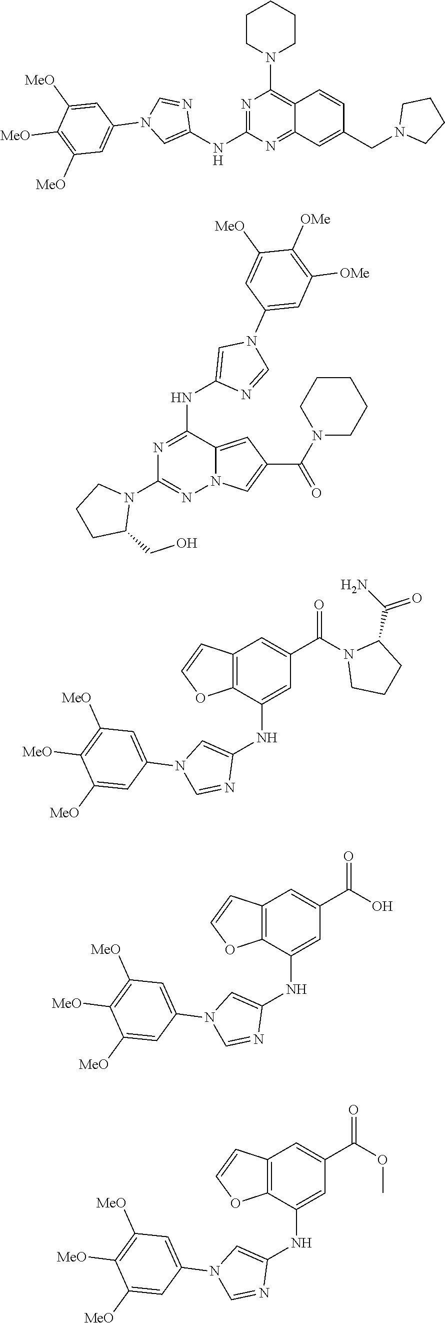 [SMILES: COc1cc(-n2cnc(Nc3nc(N4CCCCC4)c4ccc(CN5CCCC5)cc4n3)c2)cc(OC)c1OC ; COc1cc(-n2cnc(Nc3nc(N4CCC[C@H]4CO)nn4cc(C(=O)N5CCCCC5)cc34)c2)cc(OC)c1OC ; COc1cc(-n2cnc(Nc3cc(C(=O)N4CCC[C@H]4C(N)=O)cc4ccoc34)c2)cc(OC)c1OC ; COc1cc(-n2cnc(Nc3cc(C(=O)O)cc4ccoc34)c2)cc(OC)c1OC ; COC(=O)c1cc(Nc2cn(-c3cc(OC)c(OC)c(OC)c3)cn2)c2occc2c1] 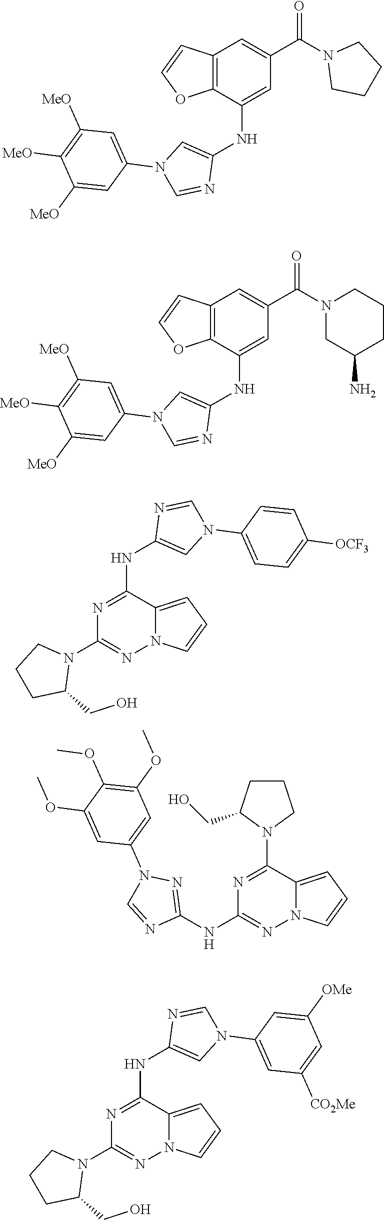 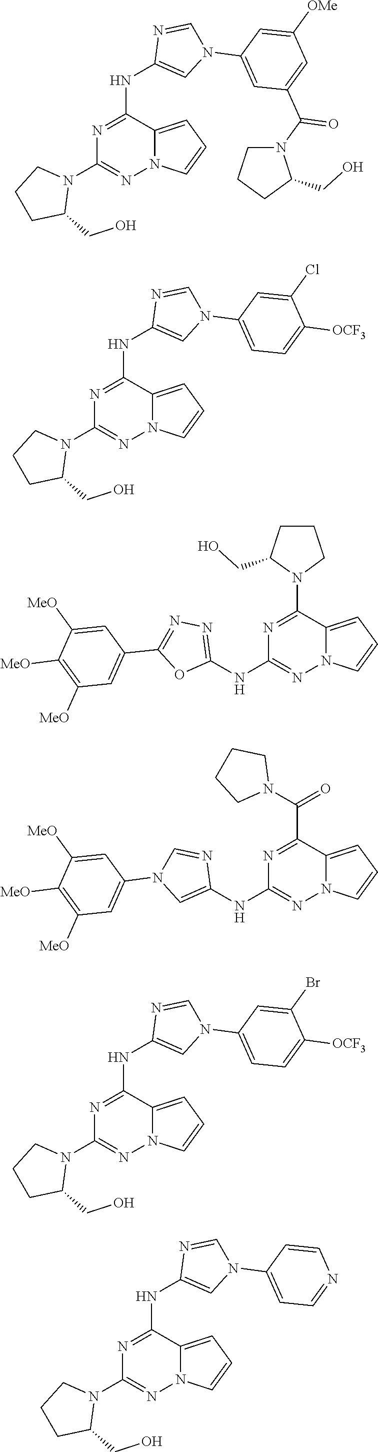 [SMILES: COc1cc(C(=O)N2CCC[C@H]2CO)cc(-n2cnc(Nc3nc(N4CCC[C@H]4CO)nn4cccc34)c2)c1 ; OC[C@@H]1CCCN1c1nc(Nc2cn(-c3ccc(OC(F)(F)F)c(Cl)c3)cn2)c2cccn2n1 ; COc1cc(-c2nnc(Nc3nc(N4CCC[C@H]4CO)c4cccn4n3)o2)cc(OC)c1OC ; COc1cc(-n2cnc(Nc3nc(C(=O)N4CCCC4)c4cccn4n3)c2)cc(OC)c1OC ; OC[C@@H]1CCCN1c1nc(Nc2cn(-c3ccc(OC(F)(F)F)c(Br)c3)cn2)c2cccn2n1 ; OC[C@@H]1CCCN1c1nc(Nc2cn(-c3ccncc3)cn2)c2cccn2n1] 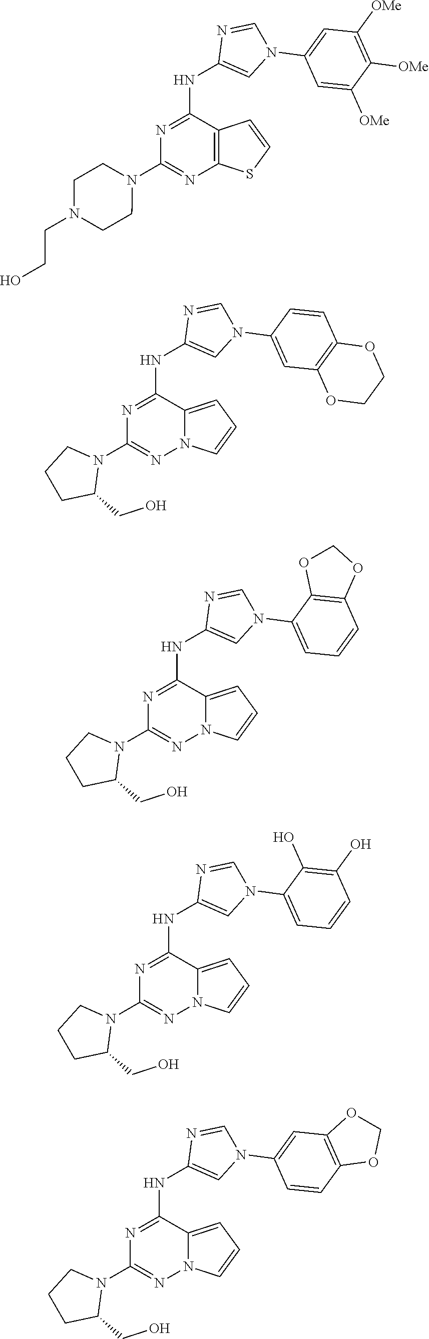 [SMILES: COc1cc(-n2cnc(Nc3nc(N4CCN(CCO)CC4)nc4sccc34)c2)cc(OC)c1OC ; OC[C@@H]1CCCN1c1nc(Nc2cn(-c3ccc4c(c3)OCCO4)cn2)c2cccn2n1 ; OC[C@@H]1CCCN1c1nc(Nc2cn(-c3cccc4c3OCO4)cn2)c2cccn2n1 ; OC[C@@H]1CCCN1c1nc(Nc2cn(-c3cccc(O)c3O)cn2)c2cccn2n1 ; OC[C@@H]1CCCN1c1nc(Nc2cn(-c3ccc4c(c3)OCO4)cn2)c2cccn2n1] 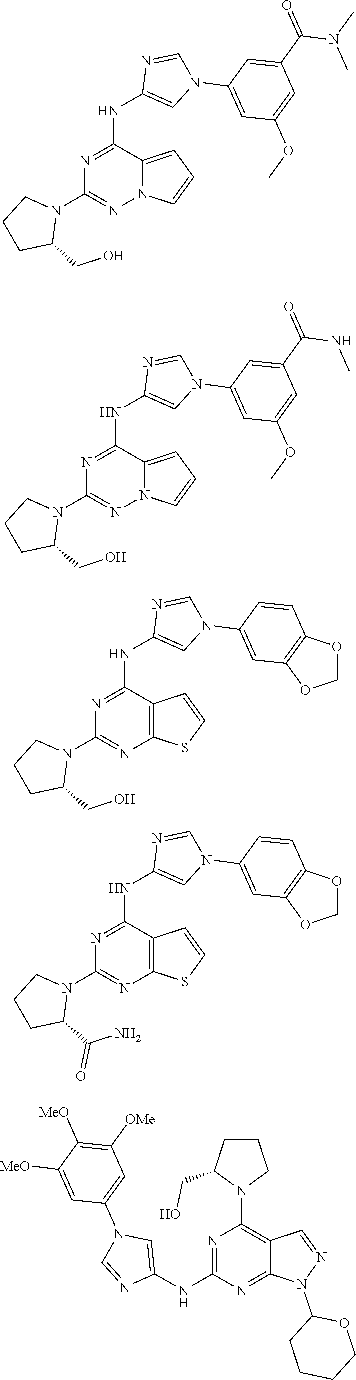 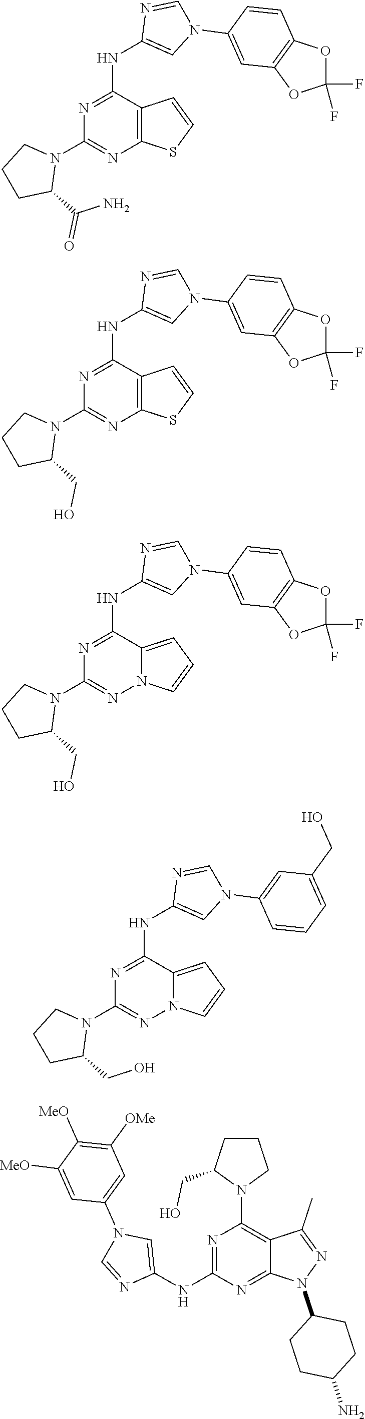 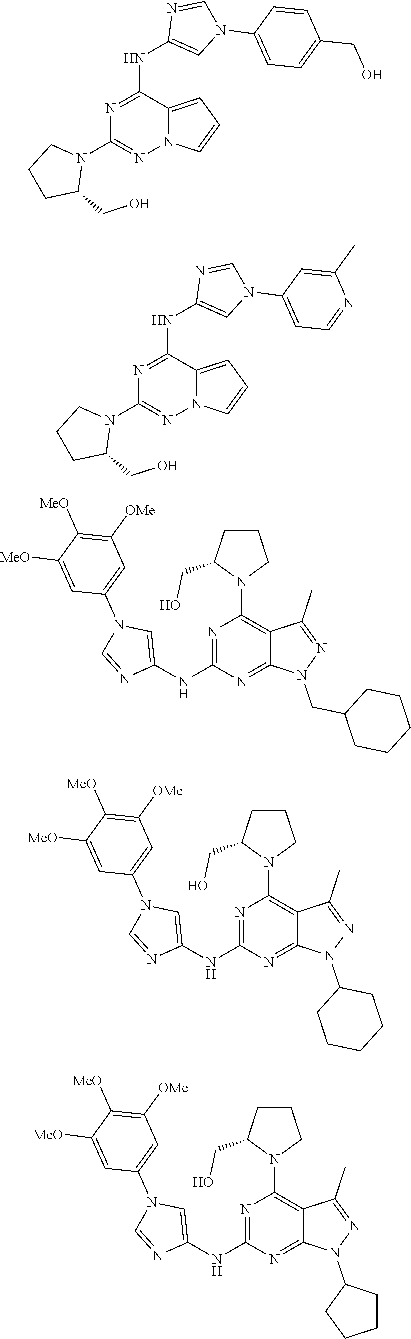 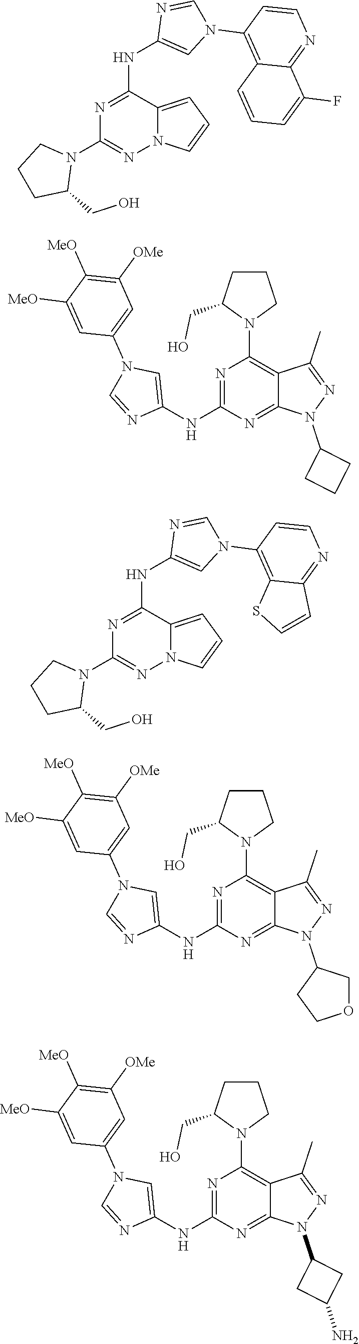 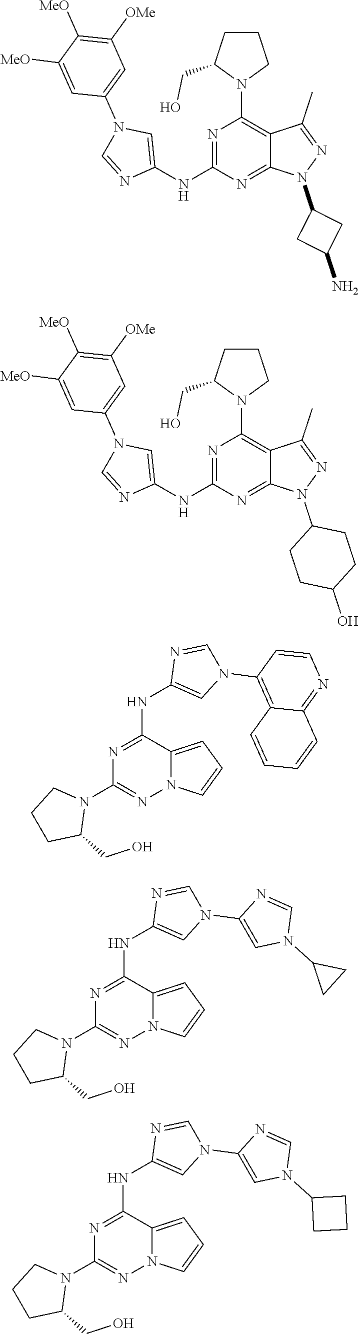 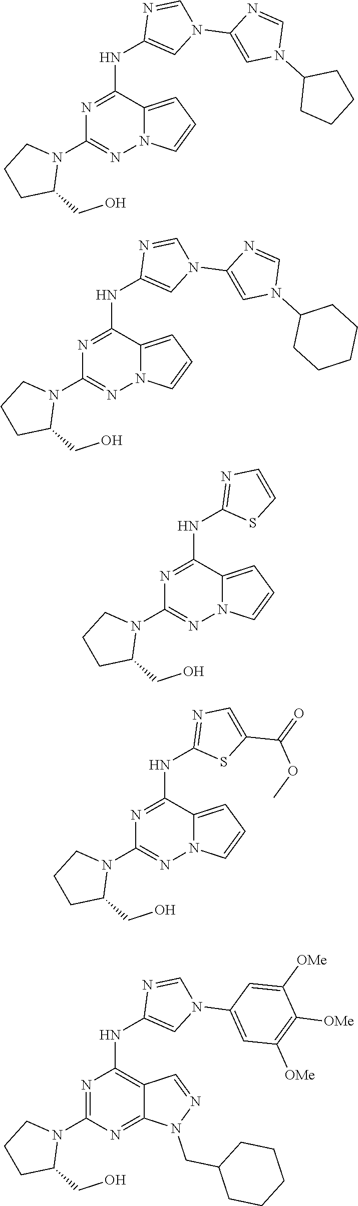 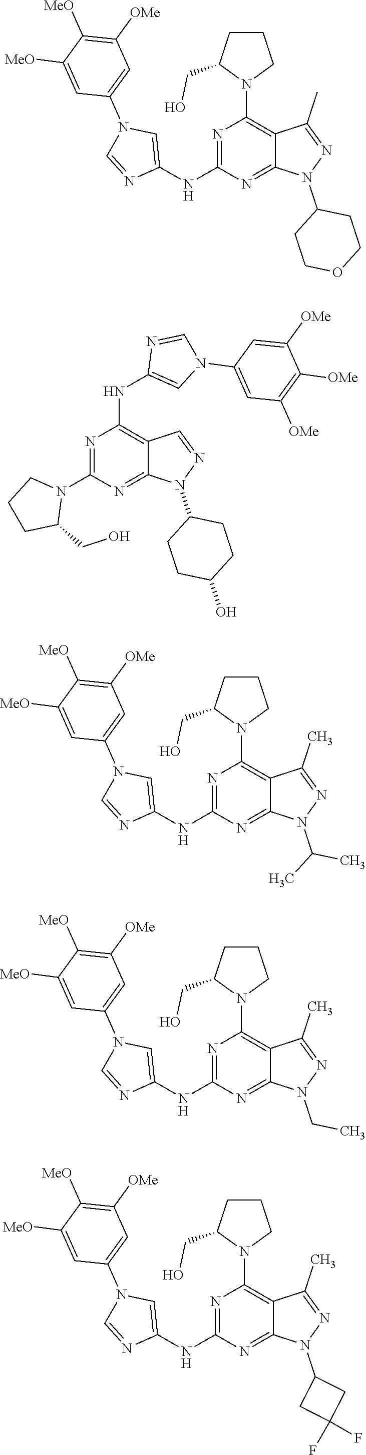 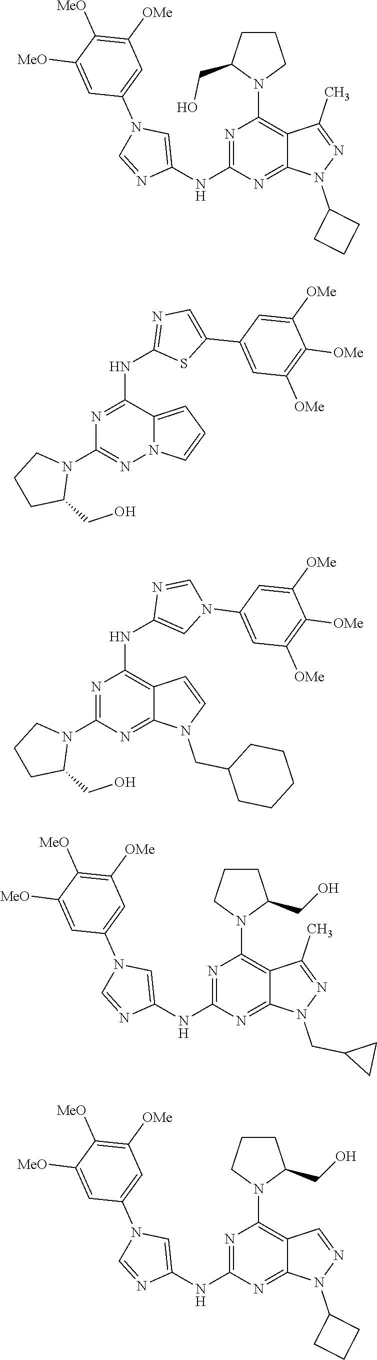 [SMILES: COc1cc(-n2cnc(Nc3nc(N4CCC[C@@H]4CO)c4c(C)nn(C5CCC5)c4n3)c2)cc(OC)c1OC ; COc1cc(-c2cnc(Nc3nc(N4CCC[C@H]4CO)nn4cccc34)s2)cc(OC)c1OC ; COc1cc(-n2cnc(Nc3nc(N4CCC[C@H]4CO)nc4c3ccn4CC3CCCCC3)c2)cc(OC)c1OC ; COc1cc(-n2cnc(Nc3nc(N4CCC[C@H]4CO)c4c(C)nn(CC5CC5)c4n3)c2)cc(OC)c1OC ; COc1cc(-n2cnc(Nc3nc(N4CCC[C@H]4CO)c4cnn(C5CCC5)c4n3)c2)cc(OC)c1OC] 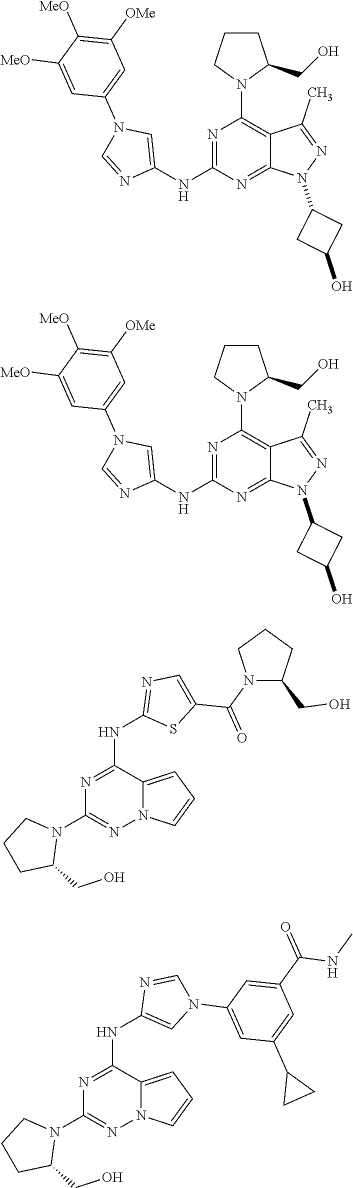 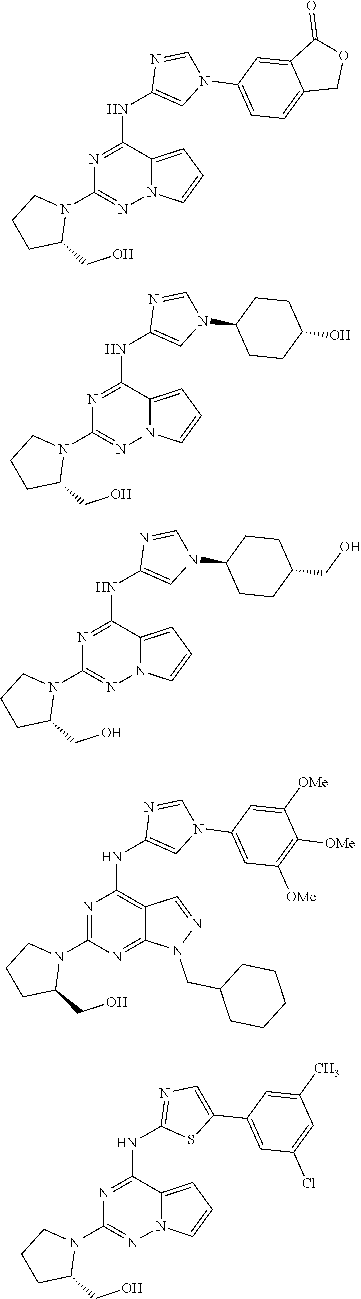 [SMILES: O=C1OCc2ccc(-n3cnc(Nc4nc(N5CCC[C@H]5CO)nn5cccc45)c3)cc21 ; OC[C@@H]1CCCN1c1nc(Nc2cn([C@H]3CC[C@H](O)CC3)cn2)c2cccn2n1 ; OC[C@@H]1CCCN1c1nc(Nc2cn([C@H]3CC[C@H](CO)CC3)cn2)c2cccn2n1 ; COc1cc(-n2cnc(Nc3nc(N4CCC[C@@H]4CO)nc4c3cnn4CC3CCCCC3)c2)cc(OC)c1OC ; Cc1cc(Cl)cc(-c2cnc(Nc3nc(N4CCC[C@H]4CO)nn4cccc34)s2)c1] 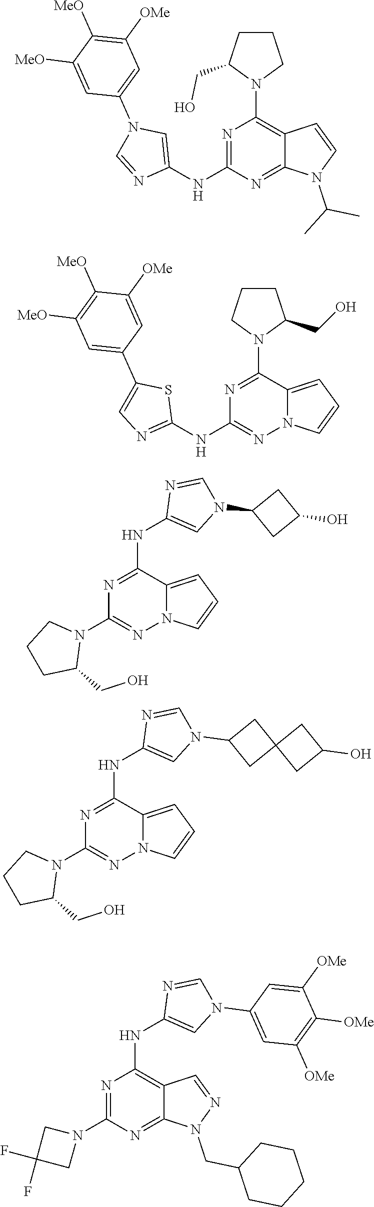 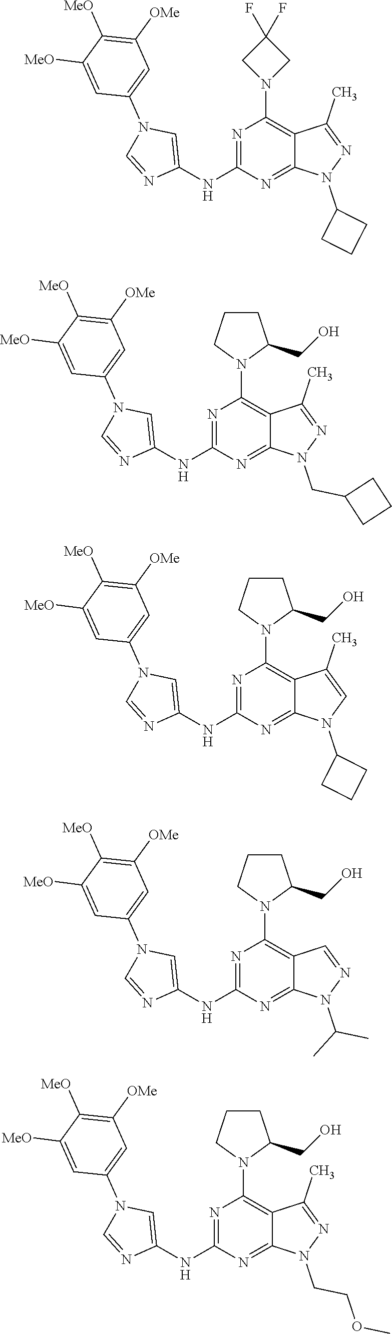 [SMILES: COc1cc(-n2cnc(Nc3nc(N4CC(F)(F)C4)c4c(C)nn(C5CCC5)c4n3)c2)cc(OC)c1OC ; COc1cc(-n2cnc(Nc3nc(N4CCC[C@H]4CO)c4c(C)nn(CC5CCC5)c4n3)c2)cc(OC)c1OC ; COc1cc(-n2cnc(Nc3nc(N4CCC[C@H]4CO)c4c(C)cn(C5CCC5)c4n3)c2)cc(OC)c1OC ; COc1cc(-n2cnc(Nc3nc(N4CCC[C@H]4CO)c4cnn(C(C)C)c4n3)c2)cc(OC)c1OC ; COCCn1nc(C)c2c(N3CCC[C@H]3CO)nc(Nc3cn(-c4cc(OC)c(OC)c(OC)c4)cn3)nc21] 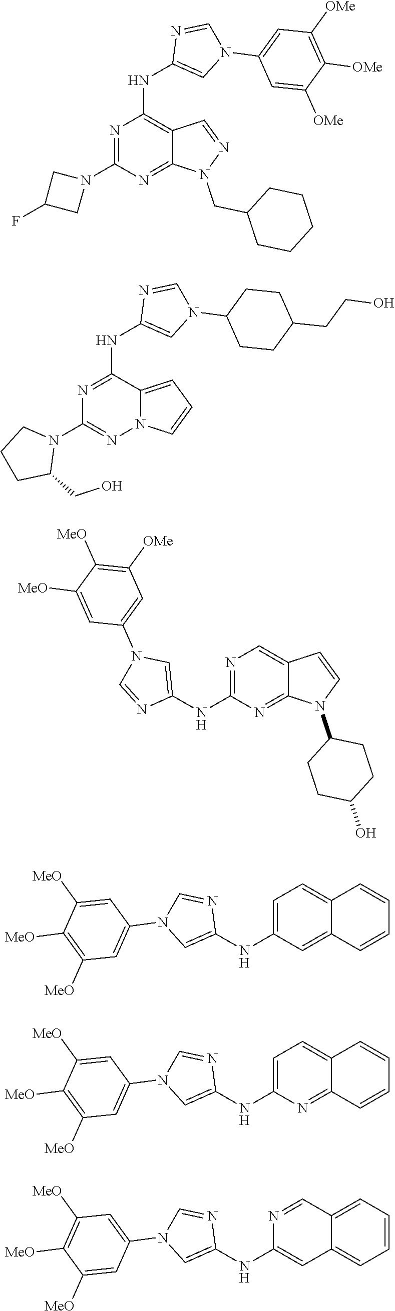 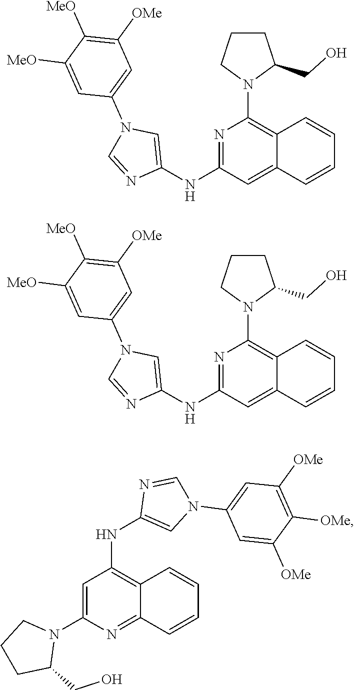 [SMILES: COc1cc(-n2cnc(Nc3cc4ccccc4c(N4CCC[C@H]4CO)n3)c2)cc(OC)c1OC ; COc1cc(-n2cnc(Nc3cc4ccccc4c(N4CCC[C@@H]4CO)n3)c2)cc(OC)c1OC ; COc1cc(-n2cnc(Nc3cc(N4CCC[C@H]4CO)nc4ccccc34)c2)cc(OC)c1OC]
or a pharmaceutically acceptable salt or prodrug thereof; and
a pharmaceutically acceptable carrier.

2. The oral dosage form of claim 1, comprising about 10 mg to about 500 mg of the compound or a pharmaceutically acceptable salt or prodrug thereof.

3. The oral dosage form of claim 1, comprising about 10 mg to about 250 mg of the compound or a pharmaceutically acceptable salt or prodrug thereof.

4. The oral dosage form of any one of claims 1-3, wherein the oral dosage form is a capsule.

5. The oral dosage form of claim 4, wherein the capsule is a hard or soft gelatin capsule.

6. A method of inhibiting ALK2 kinase, comprising orally administering to a subject in need thereof a therapeutically effective amount of a compound selected from: 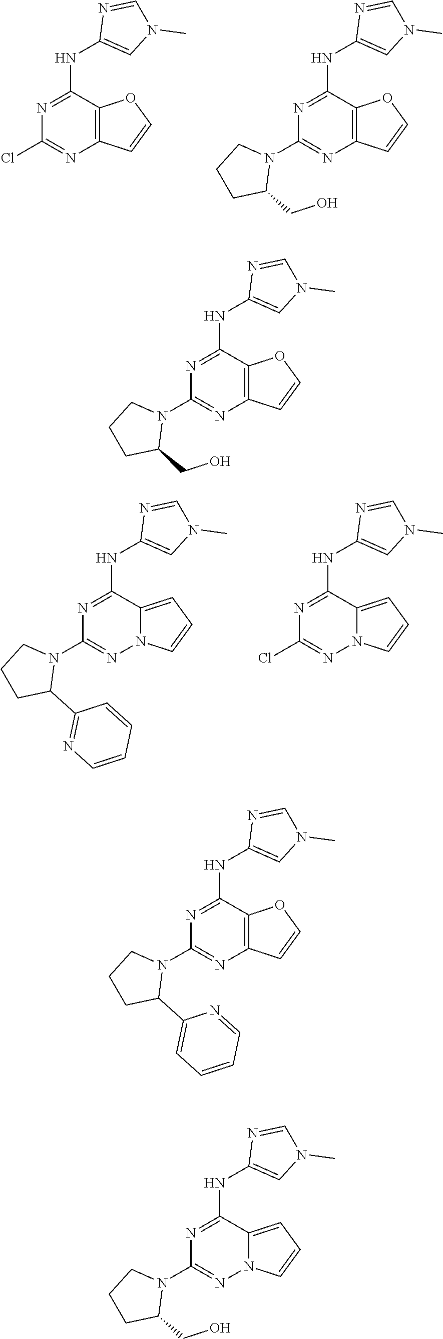 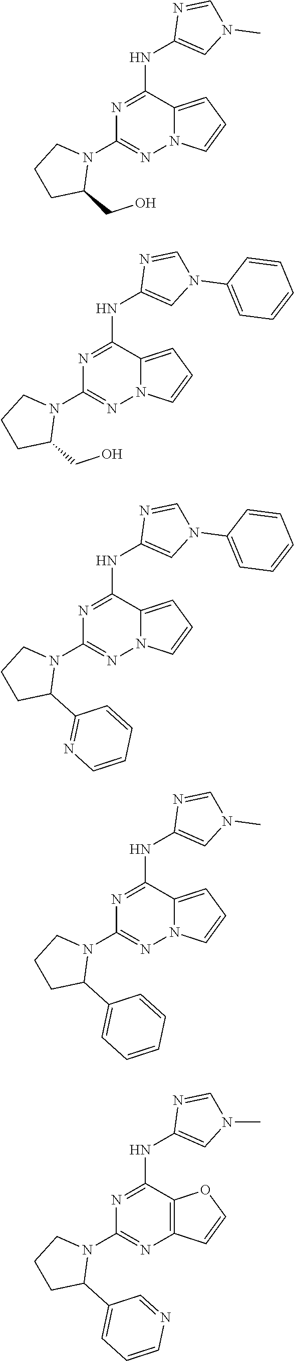 [SMILES: Cn1cnc(Nc2nc(N3CCC[C@@H]3CO)nn3cccc23)c1 ; OC[C@@H]1CCCN1c1nc(Nc2cn(-c3ccccc3)cn2)c2cccn2n1 ; c1ccc(-n2cnc(Nc3nc(N4CCCC4c4ccccn4)nn4cccc34)c2)cc1 ; Cn1cnc(Nc2nc(N3CCCC3c3ccccc3)nn3cccc23)c1 ; Cn1cnc(Nc2nc(N3CCCC3c3cccnc3)nc3ccoc23)c1] 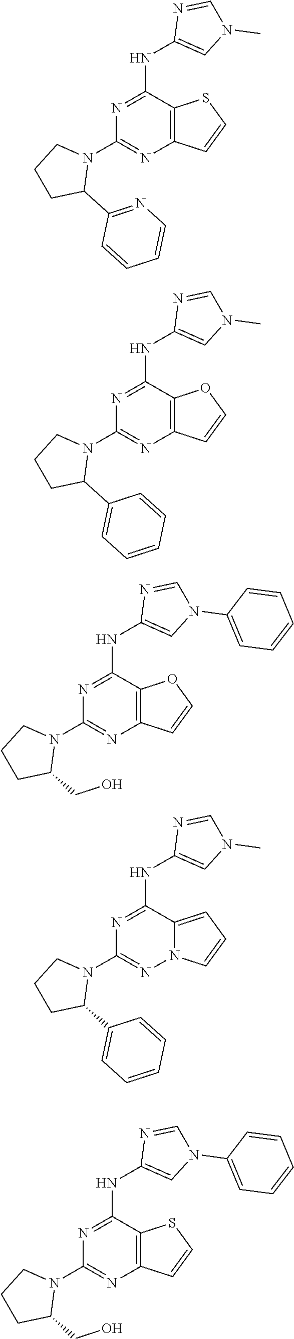 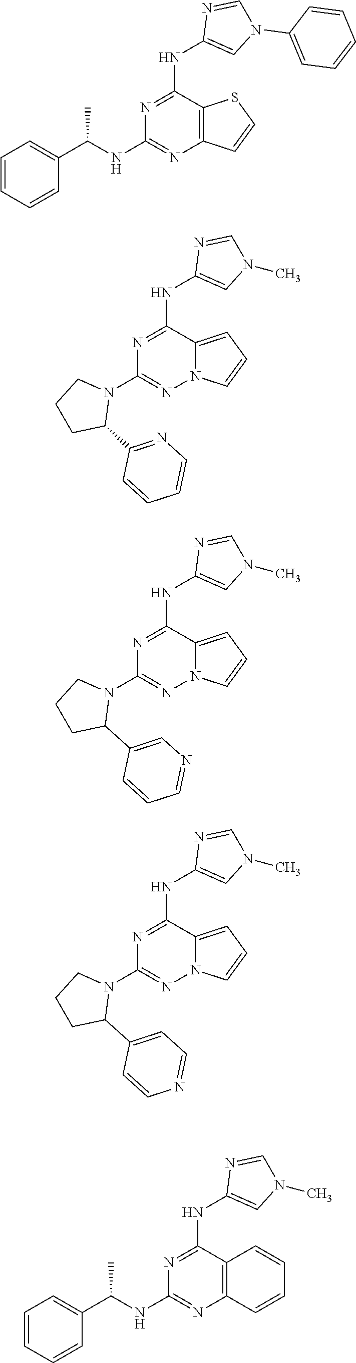 [SMILES: C[C@H](Nc1nc(Nc2cn(-c3ccccc3)cn2)c2sccc2n1)c1ccccc1 ; Cn1cnc(Nc2nc(N3CCC[C@H]3c3ccccn3)nn3cccc23)c1 ; Cn1cnc(Nc2nc(N3CCCC3c3cccnc3)nn3cccc23)c1 ; Cn1cnc(Nc2nc(N3CCCC3c3ccncc3)nn3cccc23)c1 ; C[C@H](Nc1nc(Nc2cn(C)cn2)c2ccccc2n1)c1ccccc1] 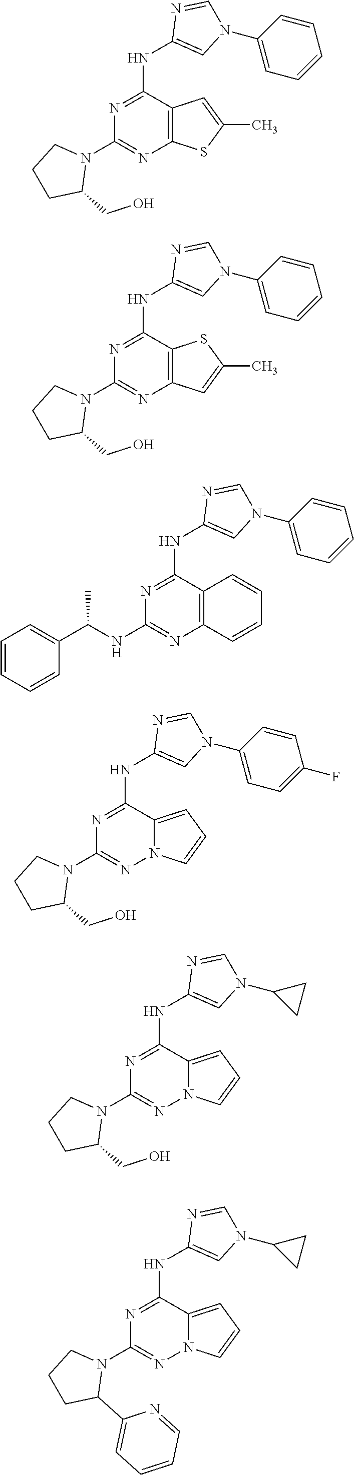 [SMILES: Cc1cc2c(Nc3cn(-c4ccccc4)cn3)nc(N3CCC[C@H]3CO)nc2s1 ; Cc1cc2nc(N3CCC[C@H]3CO)nc(Nc3cn(-c4ccccc4)cn3)c2s1 ; C[C@H](Nc1nc(Nc2cn(-c3ccccc3)cn2)c2ccccc2n1)c1ccccc1 ; OC[C@@H]1CCCN1c1nc(Nc2cn(-c3ccc(F)cc3)cn2)c2cccn2n1 ; OC[C@@H]1CCCN1c1nc(Nc2cn(C3CC3)cn2)c2cccn2n1 ; c1ccc(C2CCCN2c2nc(Nc3cn(C4CC4)cn3)c3cccn3n2)nc1] 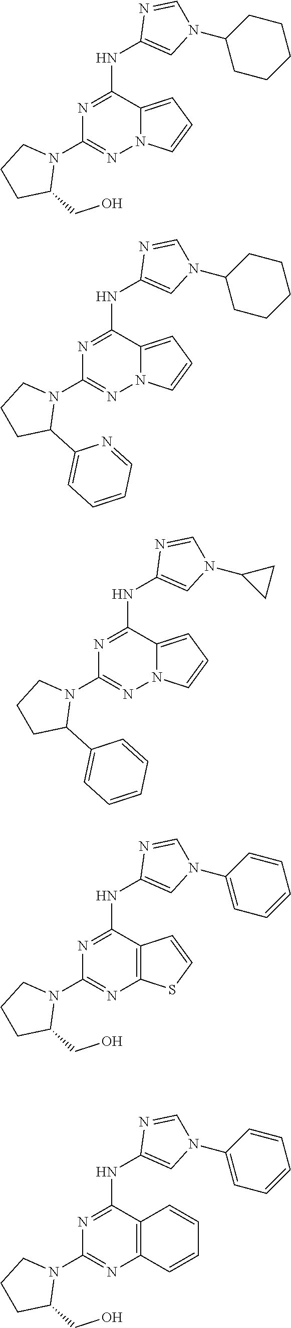 [SMILES: OC[C@@H]1CCCN1c1nc(Nc2cn(C3CCCCC3)cn2)c2cccn2n1 ; c1ccc(C2CCCN2c2nc(Nc3cn(C4CCCCC4)cn3)c3cccn3n2)nc1 ; c1ccc(C2CCCN2c2nc(Nc3cn(C4CC4)cn3)c3cccn3n2)cc1 ; OC[C@@H]1CCCN1c1nc(Nc2cn(-c3ccccc3)cn2)c2ccsc2n1 ; OC[C@@H]1CCCN1c1nc(Nc2cn(-c3ccccc3)cn2)c2ccccc2n1] 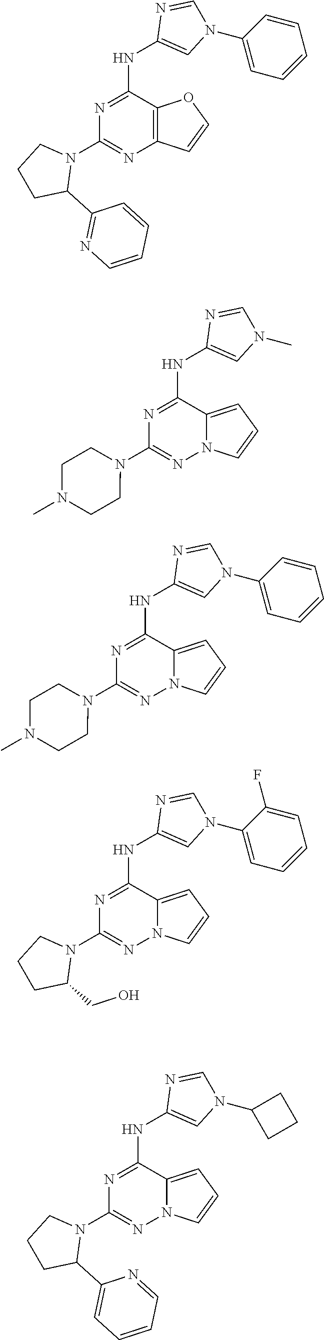 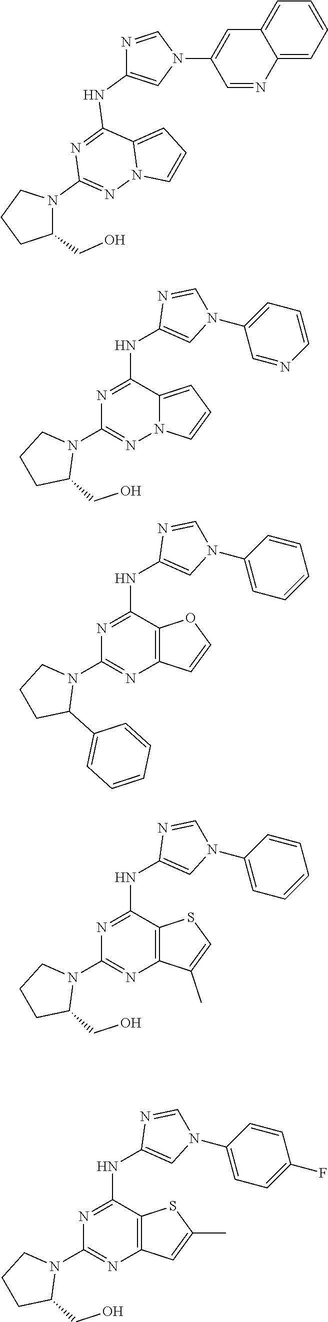 [SMILES: OC[C@@H]1CCCN1c1nc(Nc2cn(-c3cnc4ccccc4c3)cn2)c2cccn2n1 ; OC[C@@H]1CCCN1c1nc(Nc2cn(-c3cccnc3)cn2)c2cccn2n1 ; c1ccc(C2CCCN2c2nc(Nc3cn(-c4ccccc4)cn3)c3occc3n2)cc1 ; Cc1csc2c(Nc3cn(-c4ccccc4)cn3)nc(N3CCC[C@H]3CO)nc12 ; Cc1cc2nc(N3CCC[C@H]3CO)nc(Nc3cn(-c4ccc(F)cc4)cn3)c2s1] 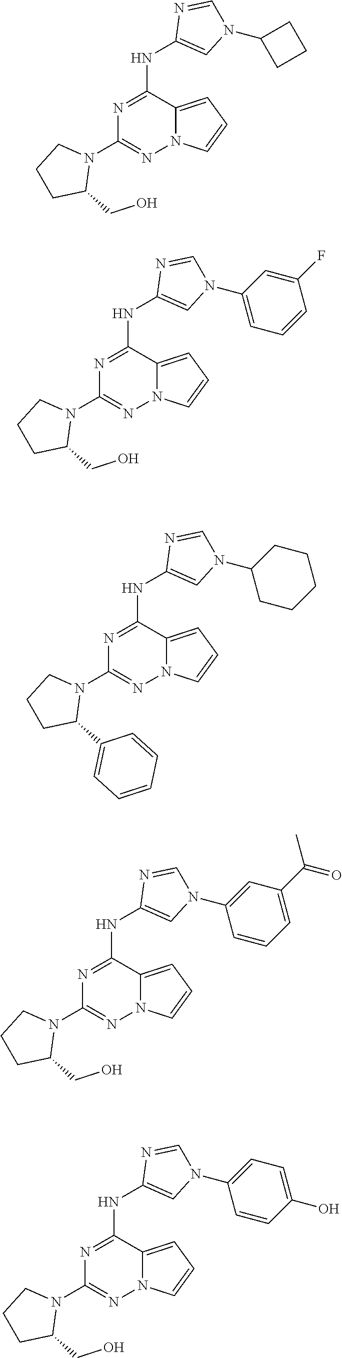 [SMILES: OC[C@@H]1CCCN1c1nc(Nc2cn(C3CCC3)cn2)c2cccn2n1 ; OC[C@@H]1CCCN1c1nc(Nc2cn(-c3cccc(F)c3)cn2)c2cccn2n1 ; c1ccc([C@@H]2CCCN2c2nc(Nc3cn(C4CCCCC4)cn3)c3cccn3n2)cc1 ; CC(=O)c1cccc(-n2cnc(Nc3nc(N4CCC[C@H]4CO)nn4cccc34)c2)c1 ; OC[C@@H]1CCCN1c1nc(Nc2cn(-c3ccc(O)cc3)cn2)c2cccn2n1] 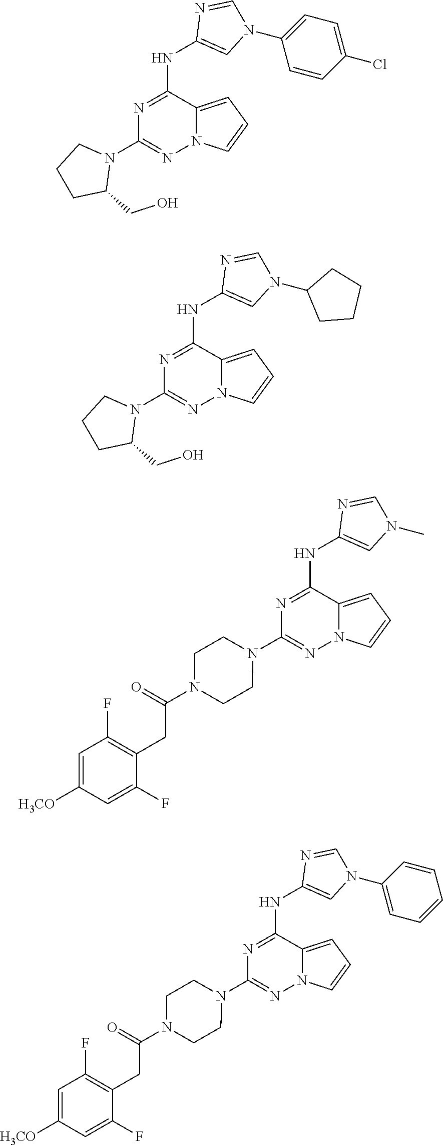 [SMILES: OC[C@@H]1CCCN1c1nc(Nc2cn(-c3ccc(Cl)cc3)cn2)c2cccn2n1 ; OC[C@@H]1CCCN1c1nc(Nc2cn(C3CCCC3)cn2)c2cccn2n1 ; COc1cc(F)c(CC(=O)N2CCN(c3nc(Nc4cn(C)cn4)c4cccn4n3)CC2)c(F)c1 ; COc1cc(F)c(CC(=O)N2CCN(c3nc(Nc4cn(-c5ccccc5)cn4)c4cccn4n3)CC2)c(F)c1] 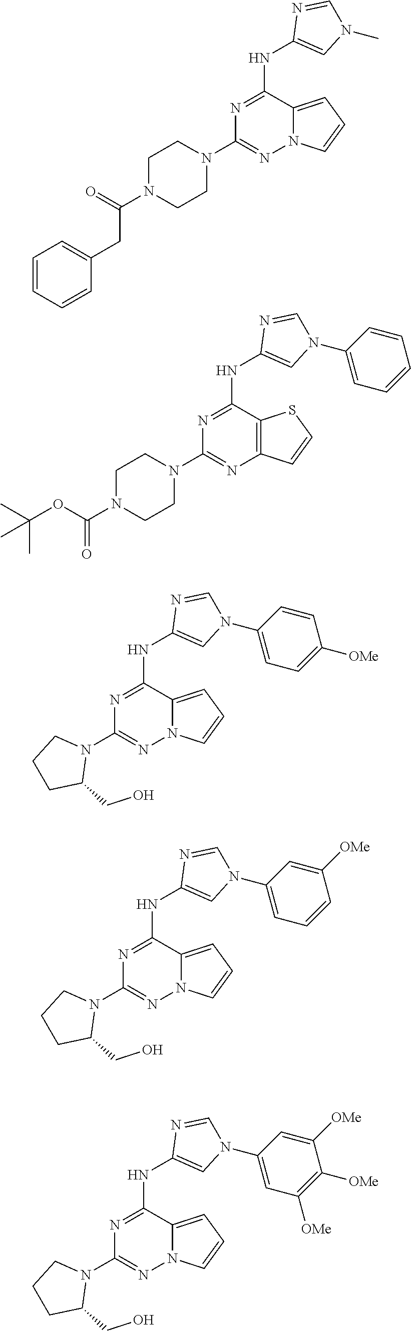 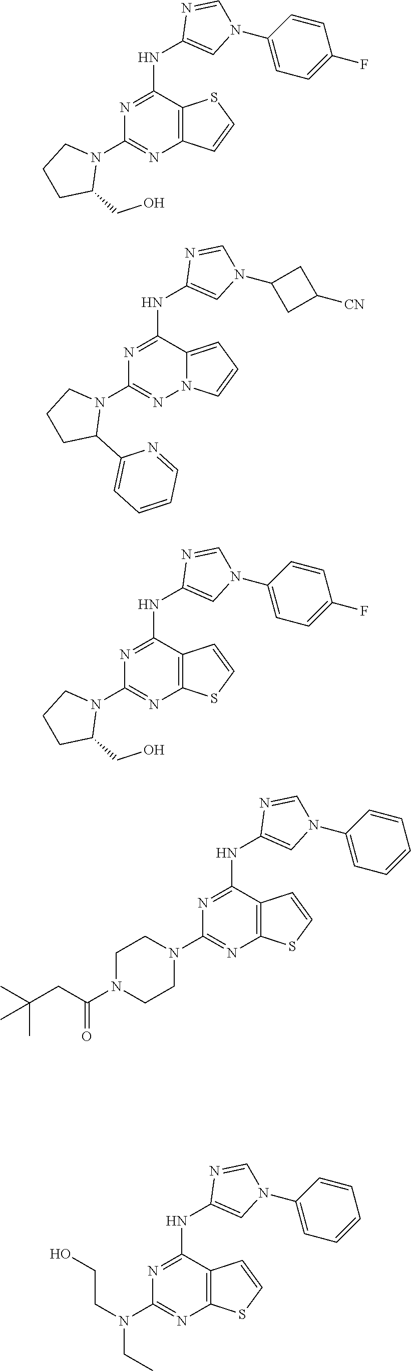 [SMILES: OC[C@@H]1CCCN1c1nc(Nc2cn(-c3ccc(F)cc3)cn2)c2sccc2n1 ; N#CC1CC(n2cnc(Nc3nc(N4CCCC4c4ccccn4)nn4cccc34)c2)C1 ; OC[C@@H]1CCCN1c1nc(Nc2cn(-c3ccc(F)cc3)cn2)c2ccsc2n1 ; CC(C)(C)CC(=O)N1CCN(c2nc(Nc3cn(-c4ccccc4)cn3)c3ccsc3n2)CC1 ; CCN(CCO)c1nc(Nc2cn(-c3ccccc3)cn2)c2ccsc2n1] 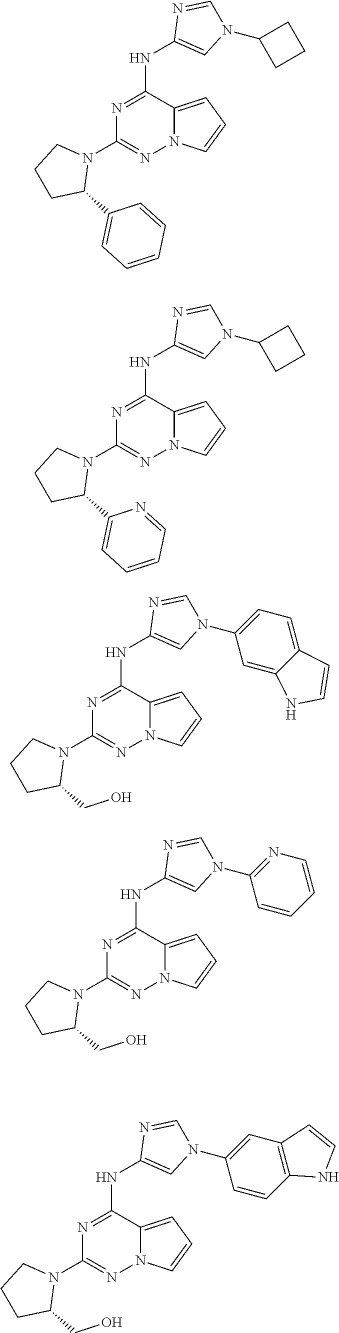 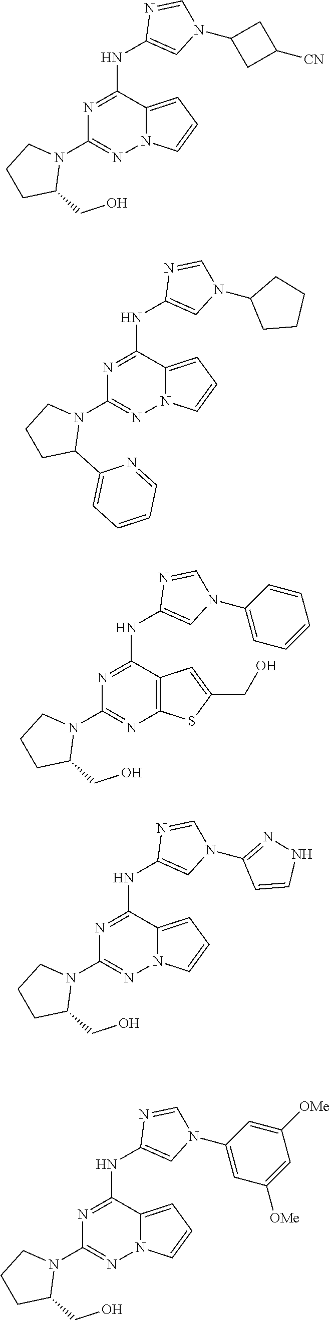 [SMILES: N#CC1CC(n2cnc(Nc3nc(N4CCC[C@H]4CO)nn4cccc34)c2)C1 ; c1ccc(C2CCCN2c2nc(Nc3cn(C4CCCC4)cn3)c3cccn3n2)nc1 ; OCc1cc2c(Nc3cn(-c4ccccc4)cn3)nc(N3CCC[C@H]3CO)nc2s1 ; OC[C@@H]1CCCN1c1nc(Nc2cn(-c3cc[nH]n3)cn2)c2cccn2n1 ; COc1cc(OC)cc(-n2cnc(Nc3nc(N4CCC[C@H]4CO)nn4cccc34)c2)c1] 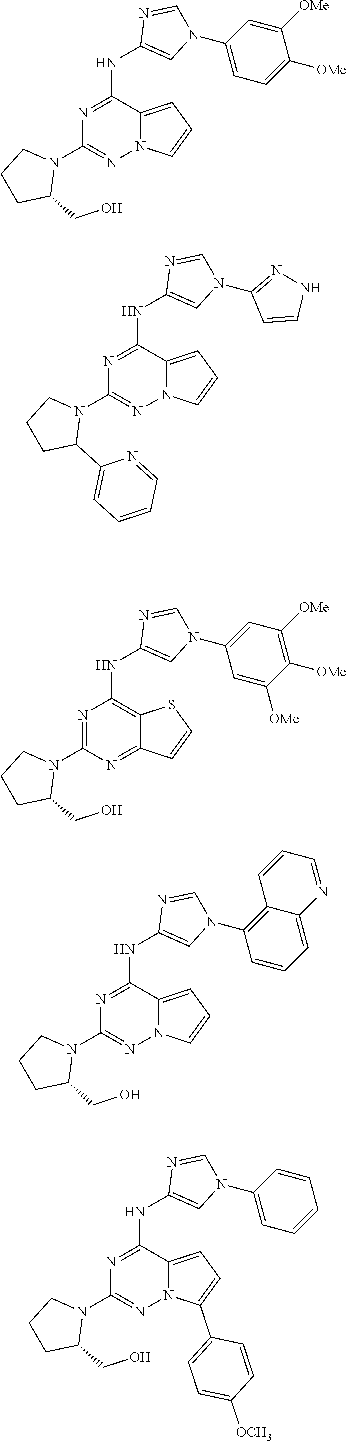 [SMILES: COc1ccc(-n2cnc(Nc3nc(N4CCC[C@H]4CO)nn4cccc34)c2)cc1OC ; c1ccc(C2CCCN2c2nc(Nc3cn(-c4cc[nH]n4)cn3)c3cccn3n2)nc1 ; COc1cc(-n2cnc(Nc3nc(N4CCC[C@H]4CO)nc4ccsc34)c2)cc(OC)c1OC ; OC[C@@H]1CCCN1c1nc(Nc2cn(-c3cccc4ncccc34)cn2)c2cccn2n1 ; COc1ccc(-c2ccc3c(Nc4cn(-c5ccccc5)cn4)nc(N4CCC[C@H]4CO)nn23)cc1] 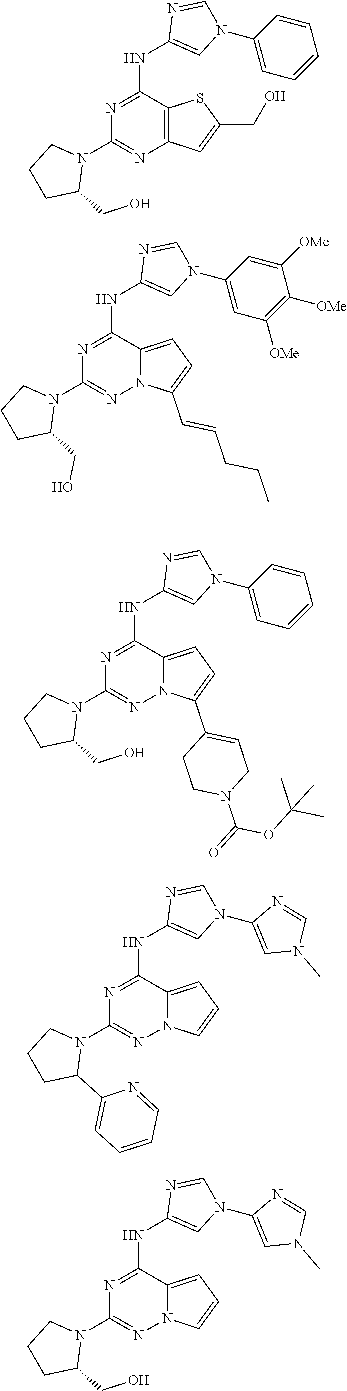 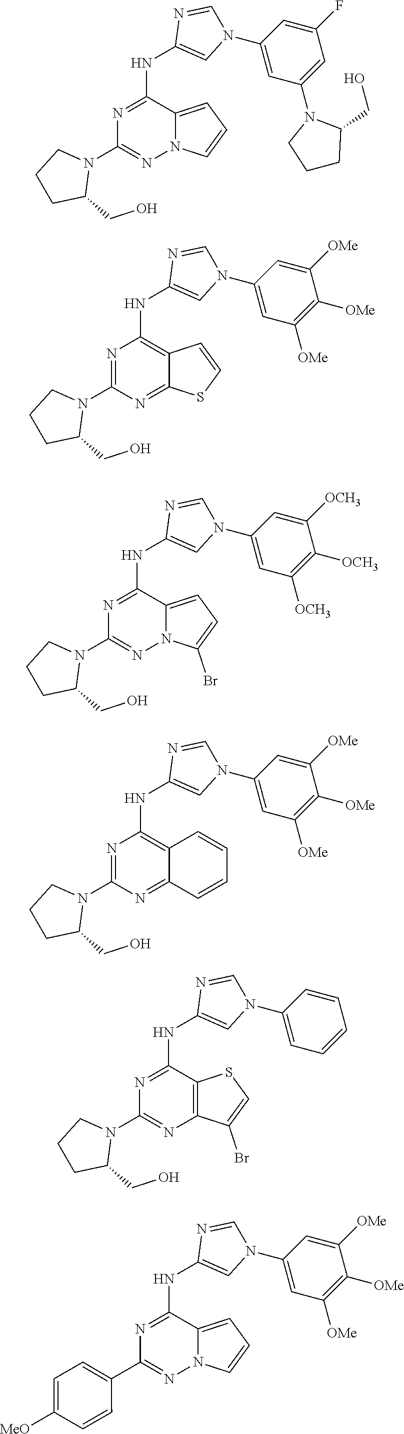 [SMILES: OC[C@@H]1CCCN1c1cc(F)cc(-n2cnc(Nc3nc(N4CCC[C@H]4CO)nn4cccc34)c2)c1 ; COc1cc(-n2cnc(Nc3nc(N4CCC[C@H]4CO)nc4sccc34)c2)cc(OC)c1OC ; COc1cc(-n2cnc(Nc3nc(N4CCC[C@H]4CO)nn4c(Br)ccc34)c2)cc(OC)c1OC ; COc1cc(-n2cnc(Nc3nc(N4CCC[C@H]4CO)nc4ccccc34)c2)cc(OC)c1OC ; OC[C@@H]1CCCN1c1nc(Nc2cn(-c3ccccc3)cn2)c2scc(Br)c2n1 ; COc1ccc(-c2nc(Nc3cn(-c4cc(OC)c(OC)c(OC)c4)cn3)c3cccn3n2)cc1] 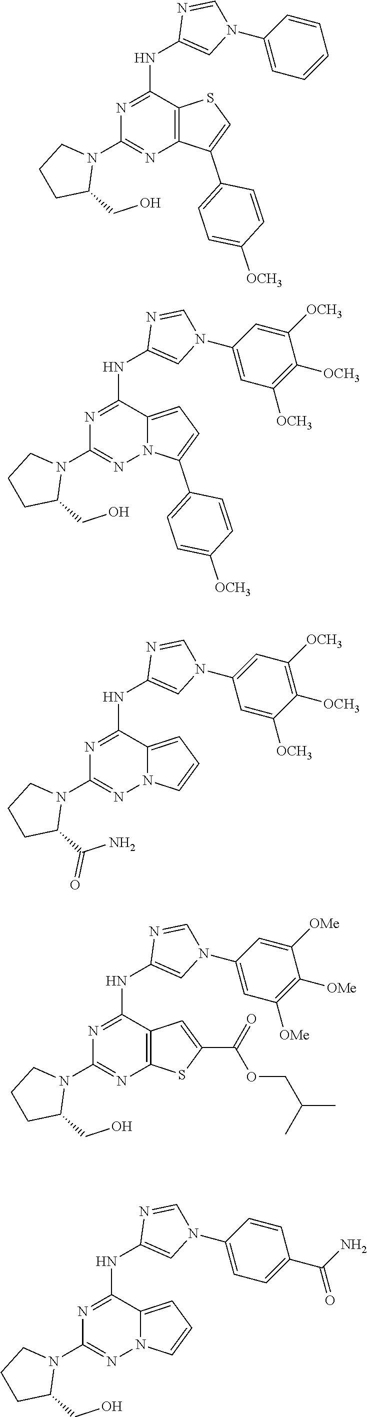 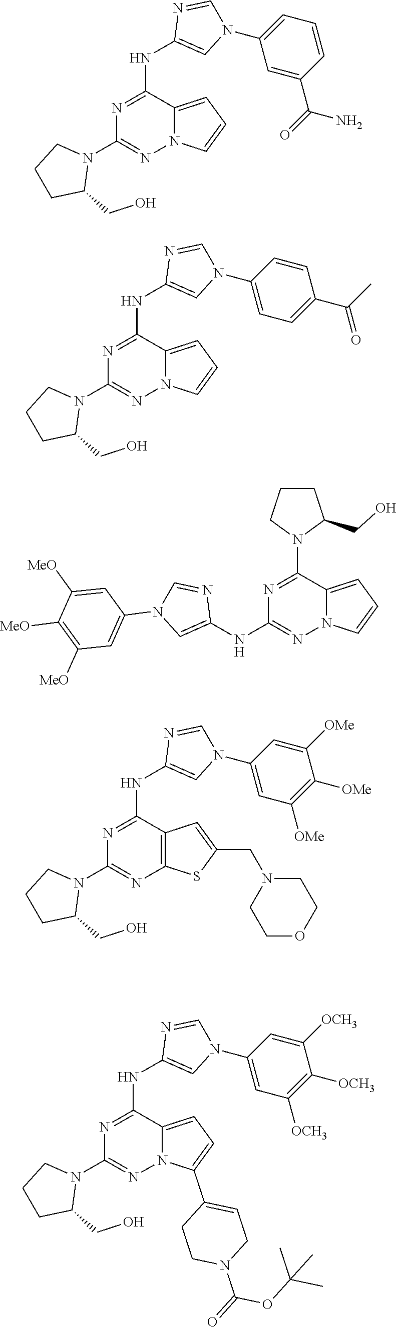 [SMILES: NC(=O)c1cccc(-n2cnc(Nc3nc(N4CCC[C@H]4CO)nn4cccc34)c2)c1 ; CC(=O)c1ccc(-n2cnc(Nc3nc(N4CCC[C@H]4CO)nn4cccc34)c2)cc1 ; COc1cc(-n2cnc(Nc3nc(N4CCC[C@H]4CO)c4cccn4n3)c2)cc(OC)c1OC ; COc1cc(-n2cnc(Nc3nc(N4CCC[C@H]4CO)nc4sc(CN5CCOCC5)cc34)c2)cc(OC)c1OC ; COc1cc(-n2cnc(Nc3nc(N4CCC[C@H]4CO)nn4c(C5=CCN(C(=O)OC(C)(C)C)CC5)ccc34)c2)cc(OC)c1OC] 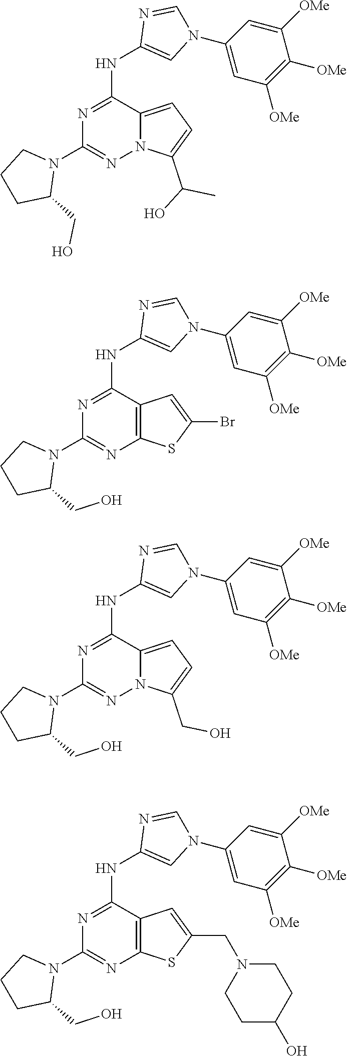 [SMILES: COc1cc(-n2cnc(Nc3nc(N4CCC[C@H]4CO)nn4c(C(C)O)ccc34)c2)cc(OC)c1OC ; COc1cc(-n2cnc(Nc3nc(N4CCC[C@H]4CO)nc4sc(Br)cc34)c2)cc(OC)c1OC ; COc1cc(-n2cnc(Nc3nc(N4CCC[C@H]4CO)nn4c(CO)ccc34)c2)cc(OC)c1OC ; COc1cc(-n2cnc(Nc3nc(N4CCC[C@H]4CO)nc4sc(CN5CCC(O)CC5)cc34)c2)cc(OC)c1OC] 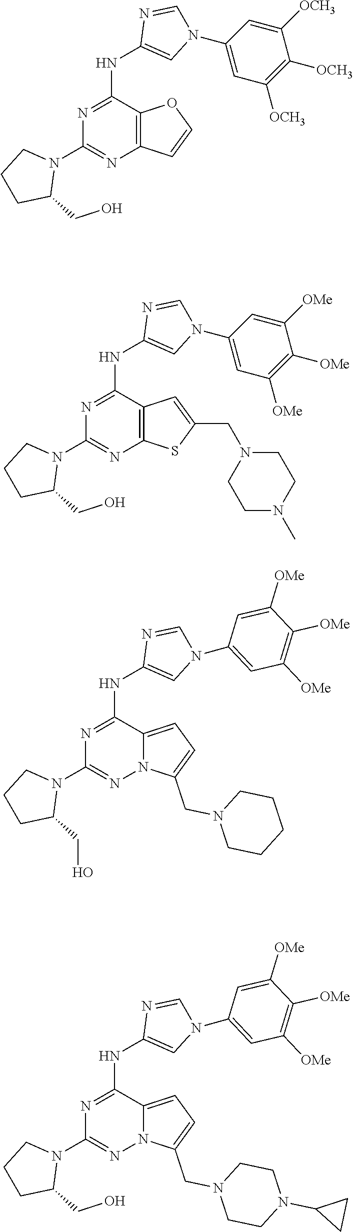 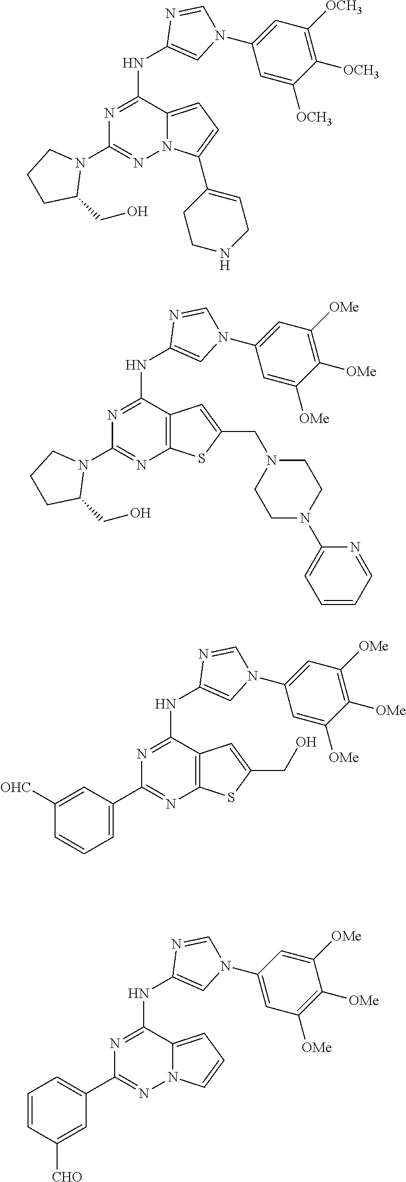 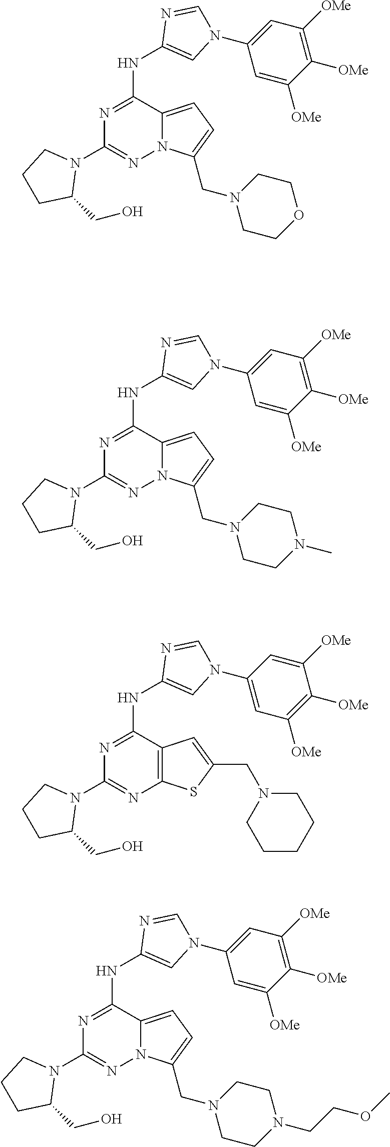 [SMILES: COc1cc(-n2cnc(Nc3nc(N4CCC[C@H]4CO)nn4c(CN5CCOCC5)ccc34)c2)cc(OC)c1OC ; COc1cc(-n2cnc(Nc3nc(N4CCC[C@H]4CO)nn4c(CN5CCN(C)CC5)ccc34)c2)cc(OC)c1OC ; COc1cc(-n2cnc(Nc3nc(N4CCC[C@H]4CO)nc4sc(CN5CCCCC5)cc34)c2)cc(OC)c1OC ; COCCN1CCN(Cc2ccc3c(Nc4cn(-c5cc(OC)c(OC)c(OC)c5)cn4)nc(N4CCC[C@H]4CO)nn23)CC1] 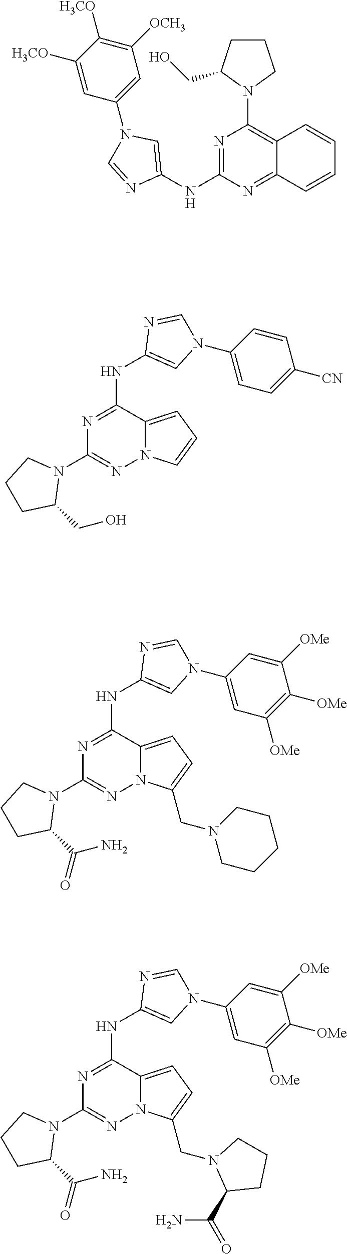 [SMILES: COc1cc(-n2cnc(Nc3nc(N4CCC[C@H]4CO)c4ccccc4n3)c2)cc(OC)c1OC ; N#Cc1ccc(-n2cnc(Nc3nc(N4CCC[C@H]4CO)nn4cccc34)c2)cc1 ; COc1cc(-n2cnc(Nc3nc(N4CCC[C@H]4C(N)=O)nn4c(CN5CCCCC5)ccc34)c2)cc(OC)c1OC ; COc1cc(-n2cnc(Nc3nc(N4CCC[C@H]4C(N)=O)nn4c(CN5CCC[C@H]5C(N)=O)ccc34)c2)cc(OC)c1OC] 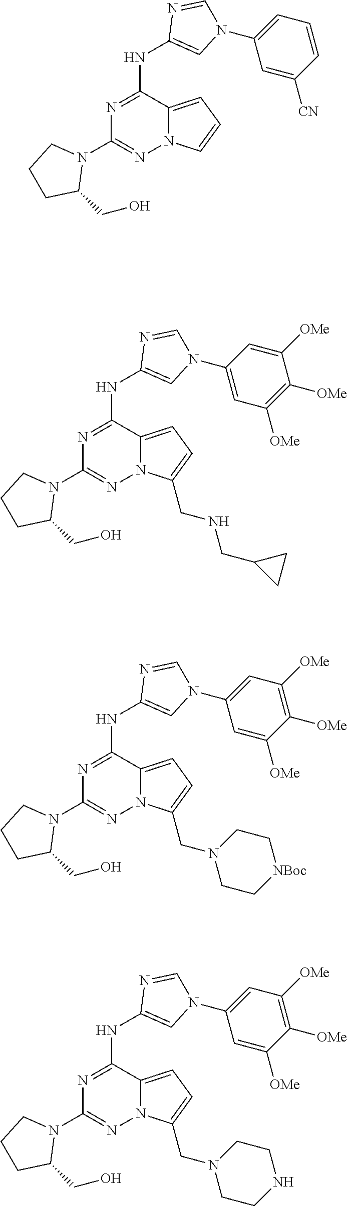 [SMILES: N#Cc1cccc(-n2cnc(Nc3nc(N4CCC[C@H]4CO)nn4cccc34)c2)c1 ; COc1cc(-n2cnc(Nc3nc(N4CCC[C@H]4CO)nn4c(CNCC5CC5)ccc34)c2)cc(OC)c1OC ; COc1cc(-n2cnc(Nc3nc(N4CCC[C@H]4CO)nn4c(CN5CCN(C(=O)OC(C)(C)C)CC5)ccc34)c2)cc(OC)c1OC ; COc1cc(-n2cnc(Nc3nc(N4CCC[C@H]4CO)nn4c(CN5CCNCC5)ccc34)c2)cc(OC)c1OC] 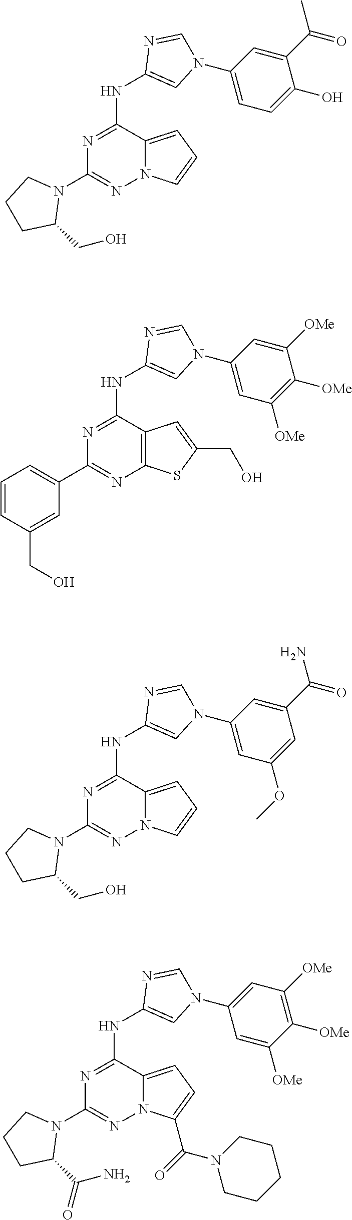 [SMILES: CC(=O)c1cc(-n2cnc(Nc3nc(N4CCC[C@H]4CO)nn4cccc34)c2)ccc1O ; COc1cc(-n2cnc(Nc3nc(-c4cccc(CO)c4)nc4sc(CO)cc34)c2)cc(OC)c1OC ; COc1cc(C(N)=O)cc(-n2cnc(Nc3nc(N4CCC[C@H]4CO)nn4cccc34)c2)c1 ; COc1cc(-n2cnc(Nc3nc(N4CCC[C@H]4C(N)=O)nn4c(C(=O)N5CCCCC5)ccc34)c2)cc(OC)c1OC] 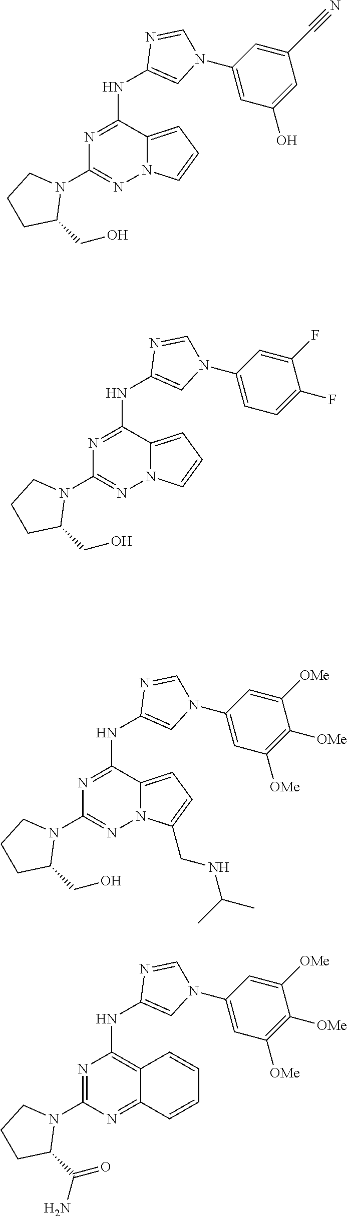 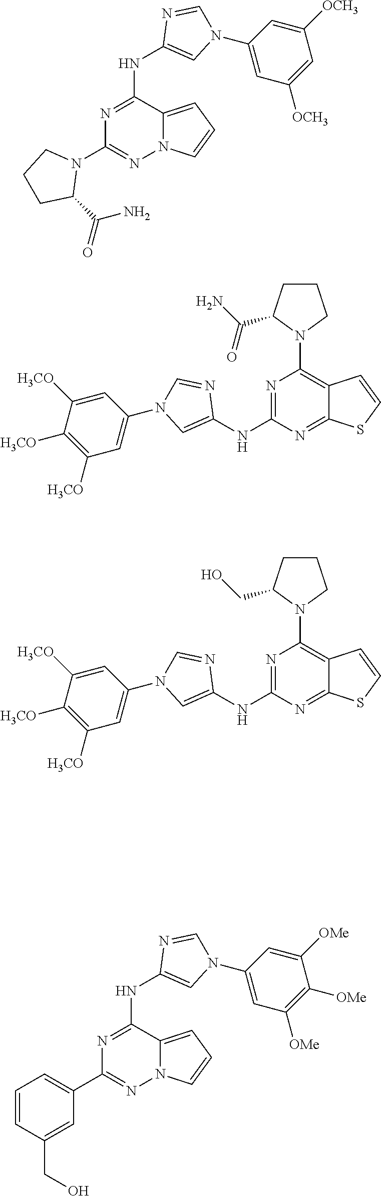 [SMILES: COc1cc(OC)cc(-n2cnc(Nc3nc(N4CCC[C@H]4C(N)=O)nn4cccc34)c2)c1 ; COc1cc(-n2cnc(Nc3nc(N4CCC[C@H]4C(N)=O)c4ccsc4n3)c2)cc(OC)c1OC ; COc1cc(-n2cnc(Nc3nc(N4CCC[C@H]4CO)c4ccsc4n3)c2)cc(OC)c1OC ; COc1cc(-n2cnc(Nc3nc(-c4cccc(CO)c4)nn4cccc34)c2)cc(OC)c1OC] 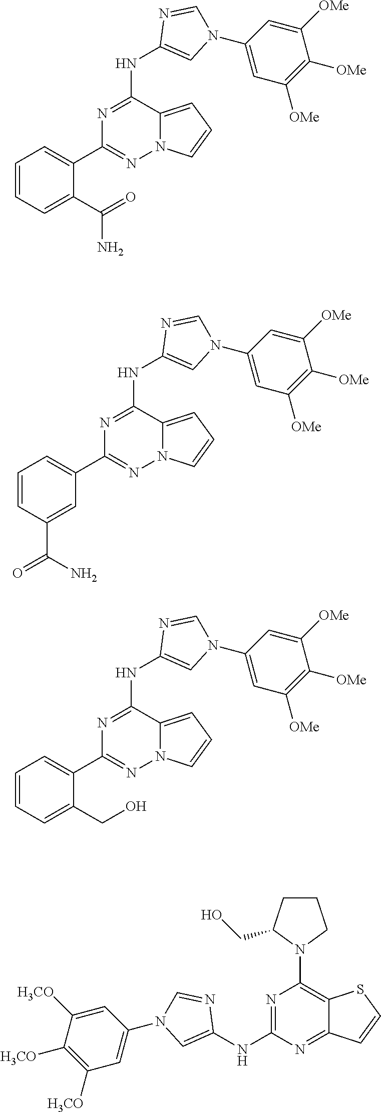 [SMILES: COc1cc(-n2cnc(Nc3nc(-c4ccccc4C(N)=O)nn4cccc34)c2)cc(OC)c1OC ; COc1cc(-n2cnc(Nc3nc(-c4cccc(C(N)=O)c4)nn4cccc34)c2)cc(OC)c1OC ; COc1cc(-n2cnc(Nc3nc(-c4ccccc4CO)nn4cccc34)c2)cc(OC)c1OC ; COc1cc(-n2cnc(Nc3nc(N4CCC[C@H]4CO)c4sccc4n3)c2)cc(OC)c1OC] 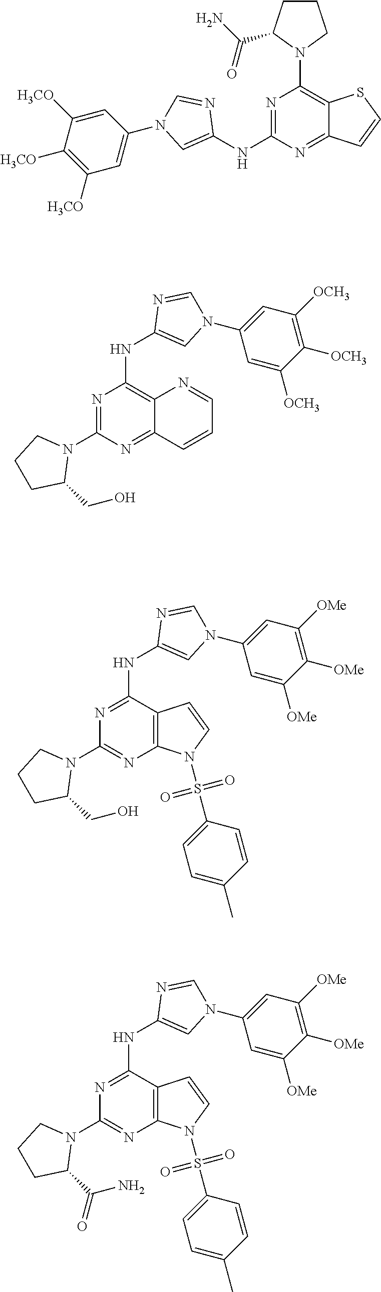 [SMILES: COc1cc(-n2cnc(Nc3nc(N4CCC[C@H]4C(N)=O)c4sccc4n3)c2)cc(OC)c1OC ; COc1cc(-n2cnc(Nc3nc(N4CCC[C@H]4CO)nc4cccnc34)c2)cc(OC)c1OC ; COc1cc(-n2cnc(Nc3nc(N4CCC[C@H]4CO)nc4c3ccn4S(=O)(=O)c3ccc(C)cc3)c2)cc(OC)c1OC ; COc1cc(-n2cnc(Nc3nc(N4CCC[C@H]4C(N)=O)nc4c3ccn4S(=O)(=O)c3ccc(C)cc3)c2)cc(OC)c1OC] 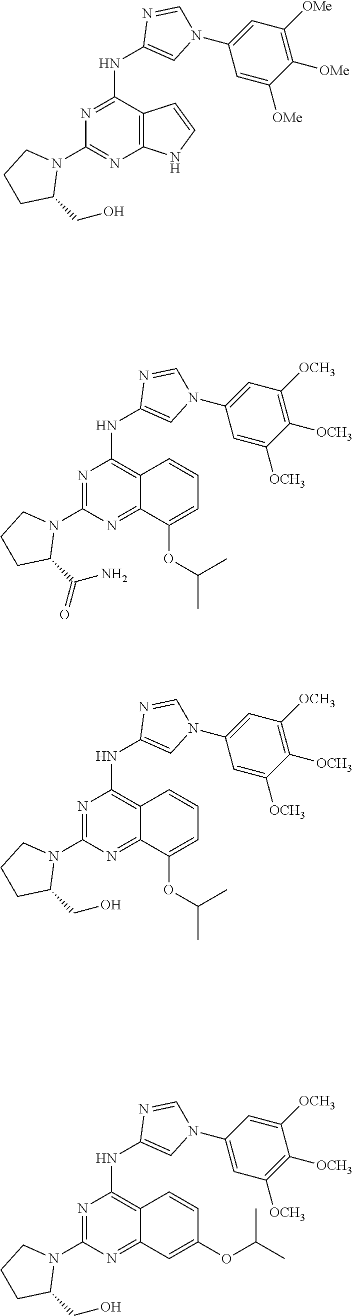 [SMILES: COc1cc(-n2cnc(Nc3nc(N4CCC[C@H]4CO)nc4[nH]ccc34)c2)cc(OC)c1OC ; COc1cc(-n2cnc(Nc3nc(N4CCC[C@H]4C(N)=O)nc4c(OC(C)C)cccc34)c2)cc(OC)c1OC ; COc1cc(-n2cnc(Nc3nc(N4CCC[C@H]4CO)nc4c(OC(C)C)cccc34)c2)cc(OC)c1OC ; COc1cc(-n2cnc(Nc3nc(N4CCC[C@H]4CO)nc4cc(OC(C)C)ccc34)c2)cc(OC)c1OC] 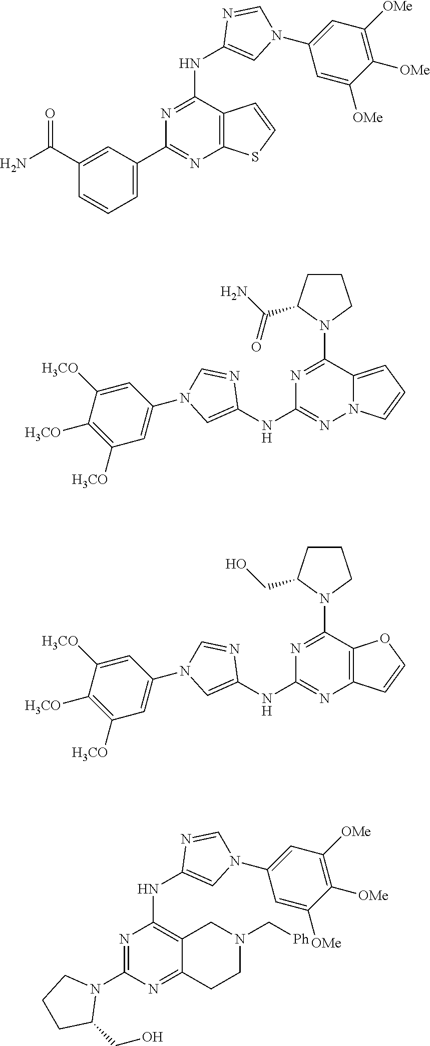 [SMILES: COc1cc(-n2cnc(Nc3nc(-c4cccc(C(N)=O)c4)nc4sccc34)c2)cc(OC)c1OC ; COc1cc(-n2cnc(Nc3nc(N4CCC[C@H]4C(N)=O)c4cccn4n3)c2)cc(OC)c1OC ; COc1cc(-n2cnc(Nc3nc(N4CCC[C@H]4CO)c4occc4n3)c2)cc(OC)c1OC ; COc1cc(-n2cnc(Nc3nc(N4CCC[C@H]4CO)nc4c3CN(Cc3ccccc3)CC4)c2)cc(OC)c1OC] 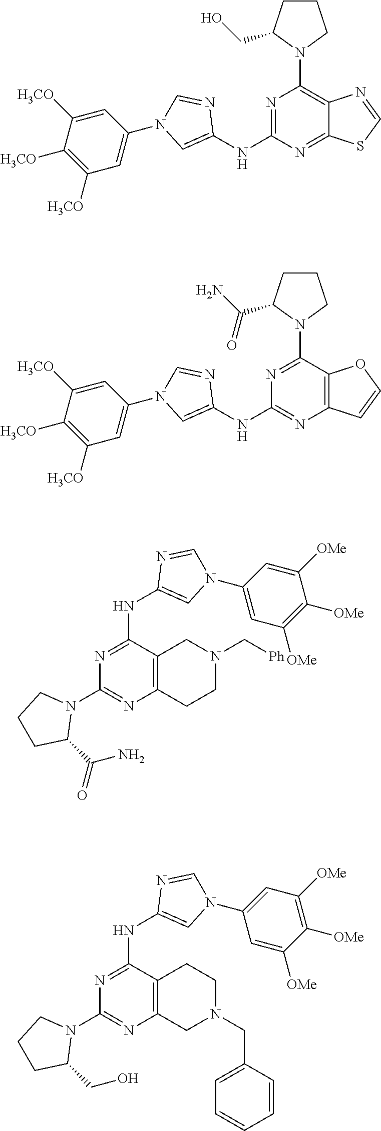 [SMILES: COc1cc(-n2cnc(Nc3nc(N4CCC[C@H]4CO)c4ncsc4n3)c2)cc(OC)c1OC ; COc1cc(-n2cnc(Nc3nc(N4CCC[C@H]4C(N)=O)c4occc4n3)c2)cc(OC)c1OC ; COc1cc(-n2cnc(Nc3nc(N4CCC[C@H]4C(N)=O)nc4c3CN(Cc3ccccc3)CC4)c2)cc(OC)c1OC ; COc1cc(-n2cnc(Nc3nc(N4CCC[C@H]4CO)nc4c3CCN(Cc3ccccc3)C4)c2)cc(OC)c1OC] 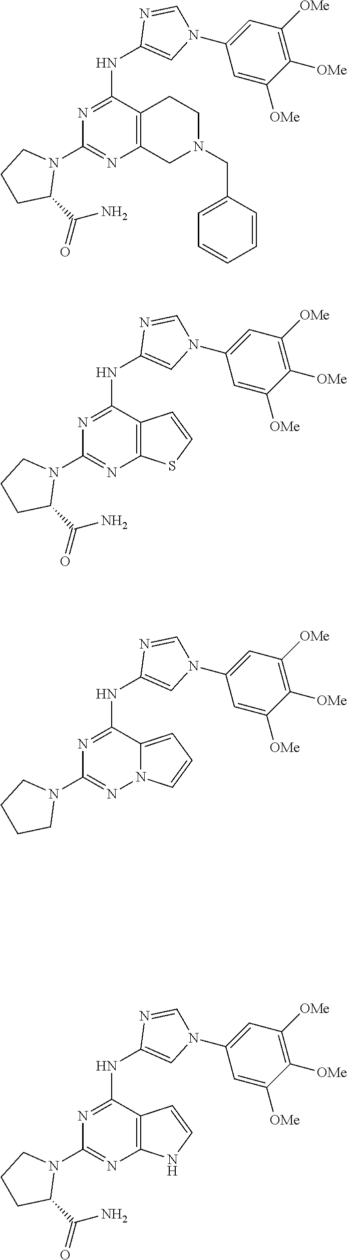 [SMILES: COc1cc(-n2cnc(Nc3nc(N4CCC[C@H]4C(N)=O)nc4c3CCN(Cc3ccccc3)C4)c2)cc(OC)c1OC ; COc1cc(-n2cnc(Nc3nc(N4CCC[C@H]4C(N)=O)nc4sccc34)c2)cc(OC)c1OC ; COc1cc(-n2cnc(Nc3nc(N4CCCC4)nn4cccc34)c2)cc(OC)c1OC ; COc1cc(-n2cnc(Nc3nc(N4CCC[C@H]4C(N)=O)nc4[nH]ccc34)c2)cc(OC)c1OC] 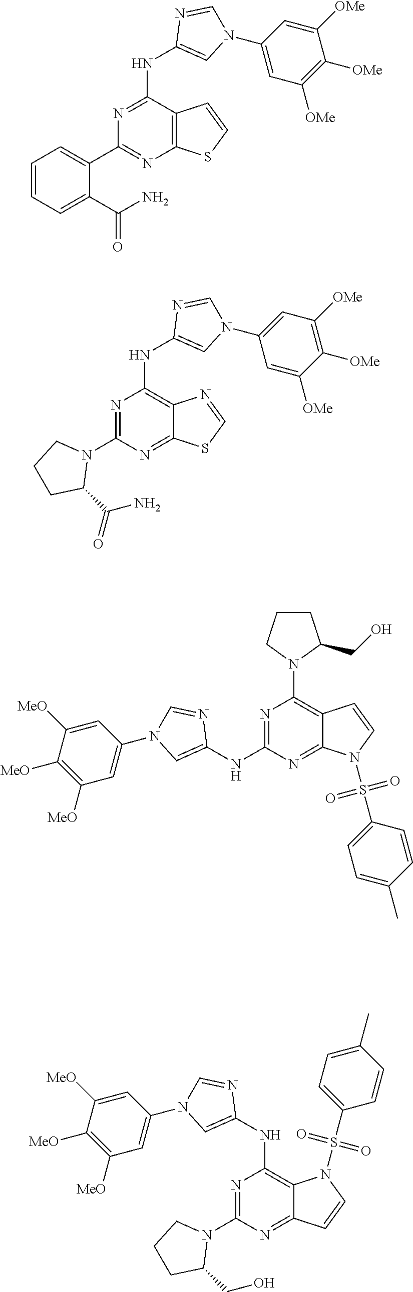 [SMILES: COc1cc(-n2cnc(Nc3nc(-c4ccccc4C(N)=O)nc4sccc34)c2)cc(OC)c1OC ; COc1cc(-n2cnc(Nc3nc(N4CCC[C@H]4C(N)=O)nc4scnc34)c2)cc(OC)c1OC ; COc1cc(-n2cnc(Nc3nc(N4CCC[C@H]4CO)c4ccn(S(=O)(=O)c5ccc(C)cc5)c4n3)c2)cc(OC)c1OC ; COc1cc(-n2cnc(Nc3nc(N4CCC[C@H]4CO)nc4ccn(S(=O)(=O)c5ccc(C)cc5)c34)c2)cc(OC)c1OC] 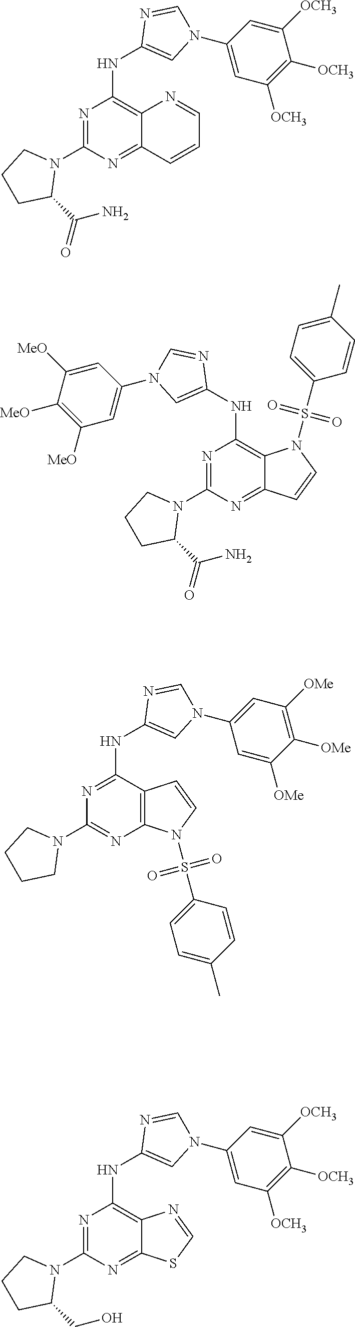 [SMILES: COc1cc(-n2cnc(Nc3nc(N4CCC[C@H]4C(N)=O)nc4cccnc34)c2)cc(OC)c1OC ; COc1cc(-n2cnc(Nc3nc(N4CCC[C@H]4C(N)=O)nc4ccn(S(=O)(=O)c5ccc(C)cc5)c34)c2)cc(OC)c1OC ; COc1cc(-n2cnc(Nc3nc(N4CCCC4)nc4c3ccn4S(=O)(=O)c3ccc(C)cc3)c2)cc(OC)c1OC ; COc1cc(-n2cnc(Nc3nc(N4CCC[C@H]4CO)nc4scnc34)c2)cc(OC)c1OC] 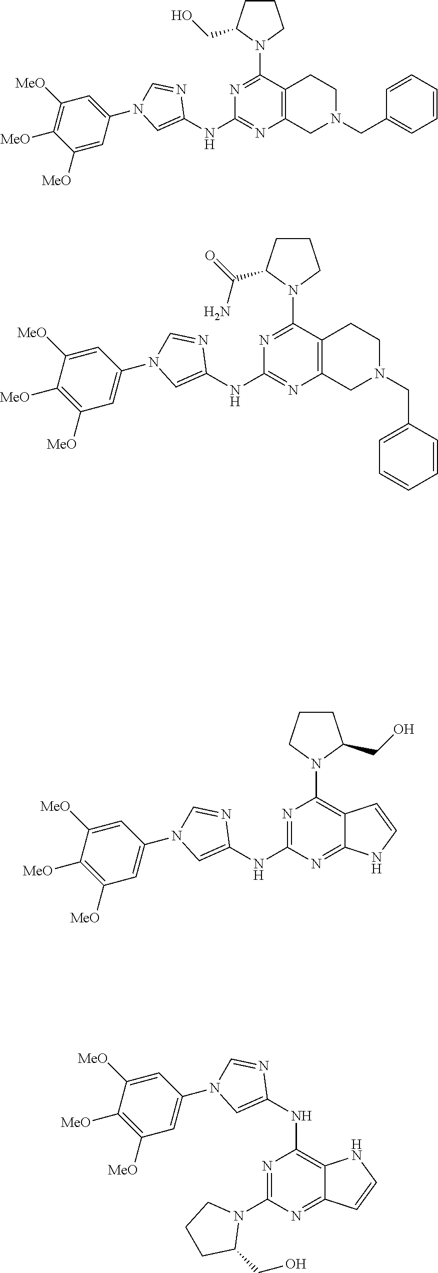 [SMILES: COc1cc(-n2cnc(Nc3nc4c(c(N5CCC[C@H]5CO)n3)CCN(Cc3ccccc3)C4)c2)cc(OC)c1OC ; COc1cc(-n2cnc(Nc3nc4c(c(N5CCC[C@H]5C(N)=O)n3)CCN(Cc3ccccc3)C4)c2)cc(OC)c1OC ; COc1cc(-n2cnc(Nc3nc(N4CCC[C@H]4CO)c4cc[nH]c4n3)c2)cc(OC)c1OC ; COc1cc(-n2cnc(Nc3nc(N4CCC[C@H]4CO)nc4cc[nH]c34)c2)cc(OC)c1OC] 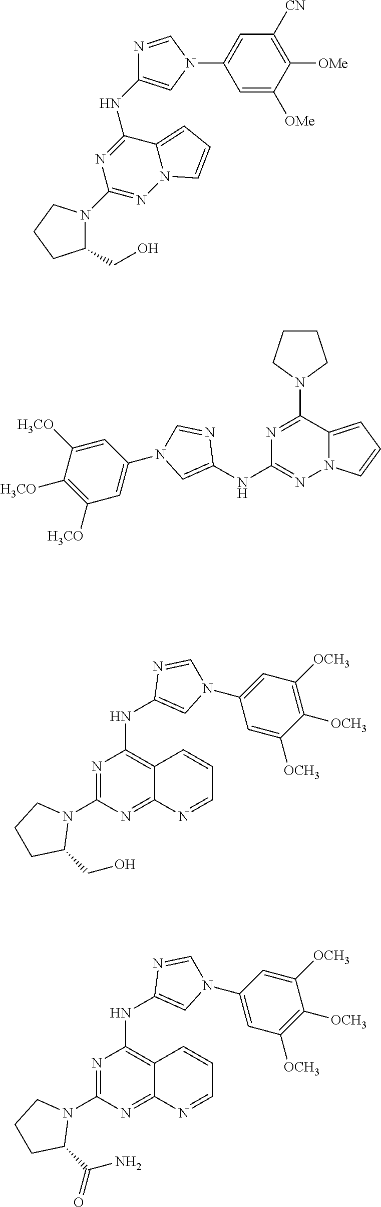 [SMILES: COc1cc(-n2cnc(Nc3nc(N4CCC[C@H]4CO)nn4cccc34)c2)cc(C#N)c1OC ; COc1cc(-n2cnc(Nc3nc(N4CCCC4)c4cccn4n3)c2)cc(OC)c1OC ; COc1cc(-n2cnc(Nc3nc(N4CCC[C@H]4CO)nc4ncccc34)c2)cc(OC)c1OC ; COc1cc(-n2cnc(Nc3nc(N4CCC[C@H]4C(N)=O)nc4ncccc34)c2)cc(OC)c1OC] 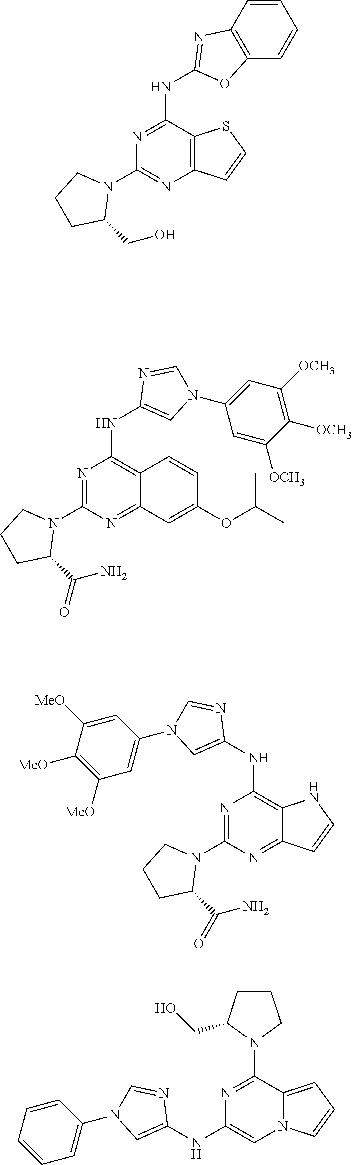 [SMILES: OC[C@@H]1CCCN1c1nc(Nc2nc3ccccc3o2)c2sccc2n1 ; COc1cc(-n2cnc(Nc3nc(N4CCC[C@H]4C(N)=O)nc4cc(OC(C)C)ccc34)c2)cc(OC)c1OC ; COc1cc(-n2cnc(Nc3nc(N4CCC[C@H]4C(N)=O)nc4cc[nH]c34)c2)cc(OC)c1OC ; OC[C@@H]1CCCN1c1nc(Nc2cn(-c3ccccc3)cn2)cn2cccc12] 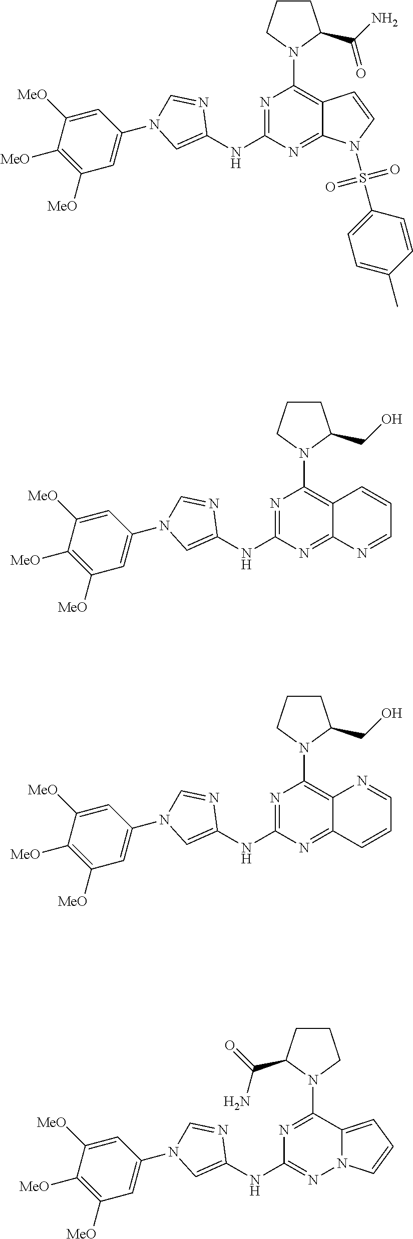 [SMILES: COc1cc(-n2cnc(Nc3nc(N4CCC[C@H]4C(N)=O)c4ccn(S(=O)(=O)c5ccc(C)cc5)c4n3)c2)cc(OC)c1OC ; COc1cc(-n2cnc(Nc3nc(N4CCC[C@H]4CO)c4cccnc4n3)c2)cc(OC)c1OC ; COc1cc(-n2cnc(Nc3nc(N4CCC[C@H]4CO)c4ncccc4n3)c2)cc(OC)c1OC ; COc1cc(-n2cnc(Nc3nc(N4CCC[C@@H]4C(N)=O)c4cccn4n3)c2)cc(OC)c1OC] 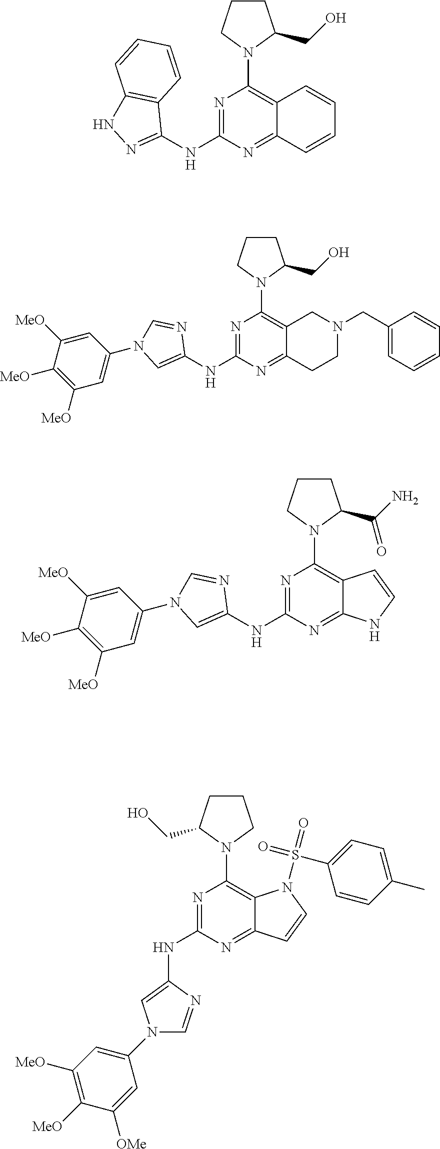 [SMILES: OC[C@@H]1CCCN1c1nc(Nc2n[nH]c3ccccc23)nc2ccccc12 ; COc1cc(-n2cnc(Nc3nc4c(c(N5CCC[C@H]5CO)n3)CN(Cc3ccccc3)CC4)c2)cc(OC)c1OC ; COc1cc(-n2cnc(Nc3nc(N4CCC[C@H]4C(N)=O)c4cc[nH]c4n3)c2)cc(OC)c1OC ; COc1cc(-n2cnc(Nc3nc(N4CCC[C@H]4CO)c4c(ccn4S(=O)(=O)c4ccc(C)cc4)n3)c2)cc(OC)c1OC] 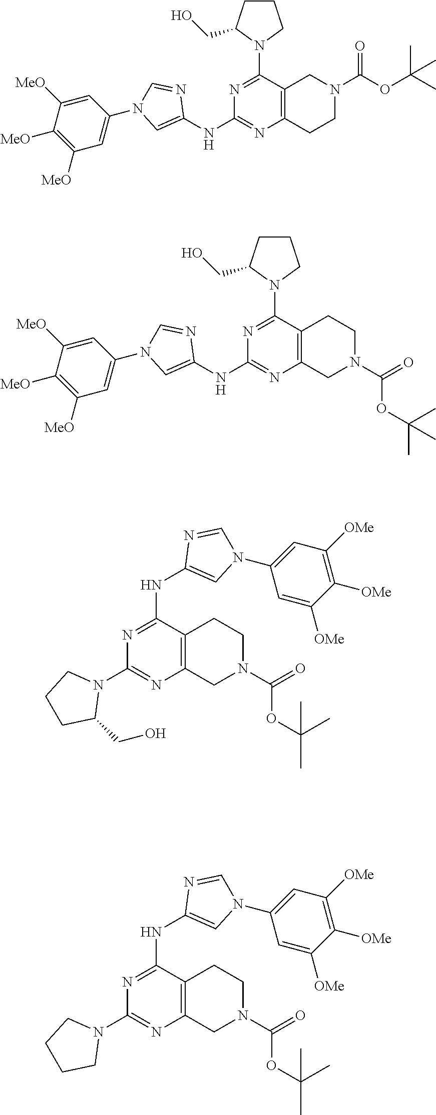 [SMILES: COc1cc(-n2cnc(Nc3nc4c(c(N5CCC[C@H]5CO)n3)CN(C(=O)OC(C)(C)C)CC4)c2)cc(OC)c1OC ; COc1cc(-n2cnc(Nc3nc4c(c(N5CCC[C@H]5CO)n3)CCN(C(=O)OC(C)(C)C)C4)c2)cc(OC)c1OC ; COc1cc(-n2cnc(Nc3nc(N4CCC[C@H]4CO)nc4c3CCN(C(=O)OC(C)(C)C)C4)c2)cc(OC)c1OC ; COc1cc(-n2cnc(Nc3nc(N4CCCC4)nc4c3CCN(C(=O)OC(C)(C)C)C4)c2)cc(OC)c1OC] 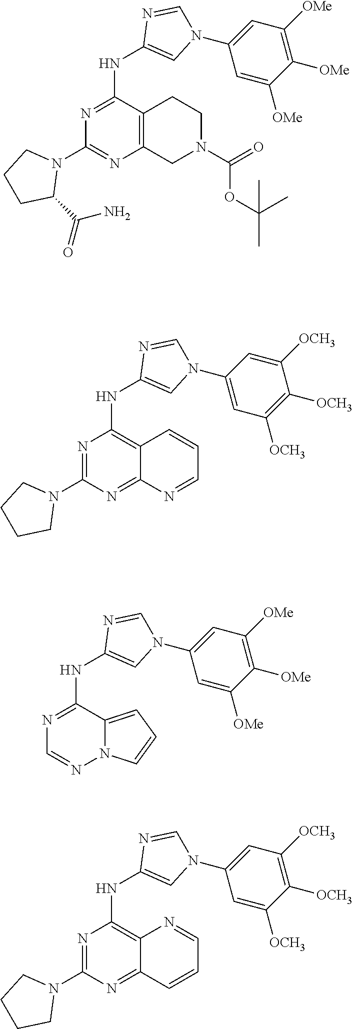 [SMILES: COc1cc(-n2cnc(Nc3nc(N4CCC[C@H]4C(N)=O)nc4c3CCN(C(=O)OC(C)(C)C)C4)c2)cc(OC)c1OC ; COc1cc(-n2cnc(Nc3nc(N4CCCC4)nc4ncccc34)c2)cc(OC)c1OC ; COc1cc(-n2cnc(Nc3ncnn4cccc34)c2)cc(OC)c1OC ; COc1cc(-n2cnc(Nc3nc(N4CCCC4)nc4cccnc34)c2)cc(OC)c1OC] 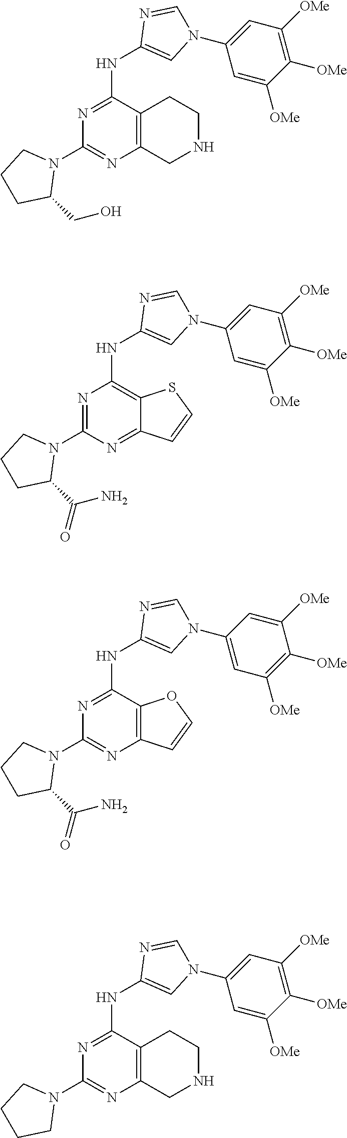 [SMILES: COc1cc(-n2cnc(Nc3nc(N4CCC[C@H]4CO)nc4c3CCNC4)c2)cc(OC)c1OC ; COc1cc(-n2cnc(Nc3nc(N4CCC[C@H]4C(N)=O)nc4ccsc34)c2)cc(OC)c1OC ; COc1cc(-n2cnc(Nc3nc(N4CCC[C@H]4C(N)=O)nc4ccoc34)c2)cc(OC)c1OC ; COc1cc(-n2cnc(Nc3nc(N4CCCC4)nc4c3CCNC4)c2)cc(OC)c1OC] 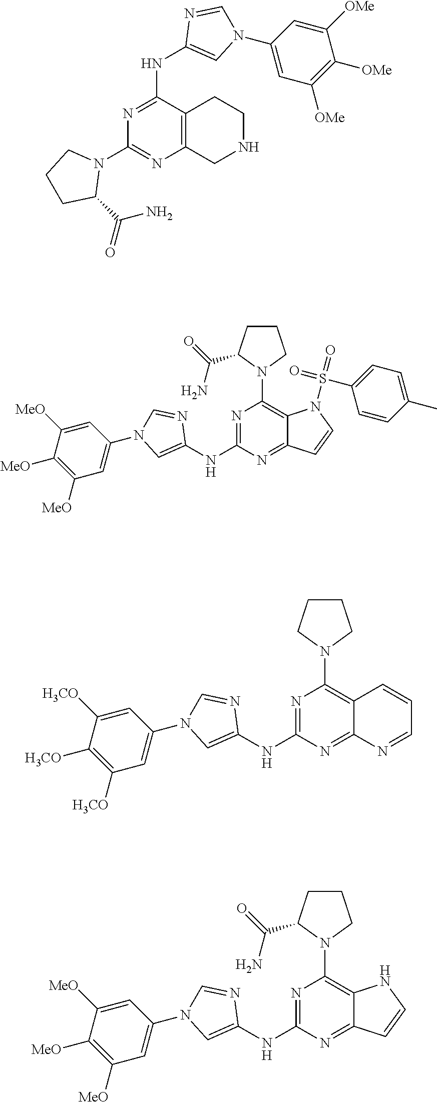 [SMILES: COc1cc(-n2cnc(Nc3nc(N4CCC[C@H]4C(N)=O)nc4c3CCNC4)c2)cc(OC)c1OC ; COc1cc(-n2cnc(Nc3nc(N4CCC[C@H]4C(N)=O)c4c(ccn4S(=O)(=O)c4ccc(C)cc4)n3)c2)cc(OC)c1OC ; COc1cc(-n2cnc(Nc3nc(N4CCCC4)c4cccnc4n3)c2)cc(OC)c1OC ; COc1cc(-n2cnc(Nc3nc(N4CCC[C@H]4C(N)=O)c4[nH]ccc4n3)c2)cc(OC)c1OC] 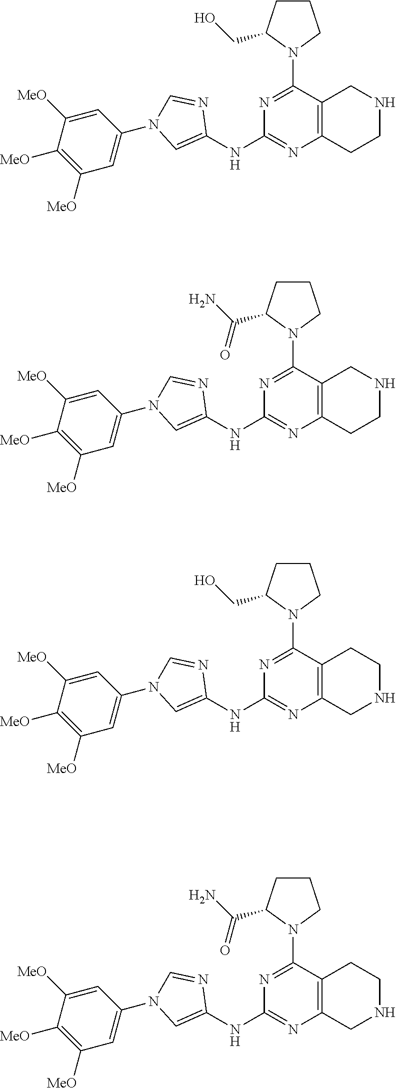 [SMILES: COc1cc(-n2cnc(Nc3nc4c(c(N5CCC[C@H]5CO)n3)CNCC4)c2)cc(OC)c1OC ; COc1cc(-n2cnc(Nc3nc4c(c(N5CCC[C@H]5C(N)=O)n3)CNCC4)c2)cc(OC)c1OC ; COc1cc(-n2cnc(Nc3nc4c(c(N5CCC[C@H]5CO)n3)CCNC4)c2)cc(OC)c1OC ; COc1cc(-n2cnc(Nc3nc4c(c(N5CCC[C@H]5C(N)=O)n3)CCNC4)c2)cc(OC)c1OC] 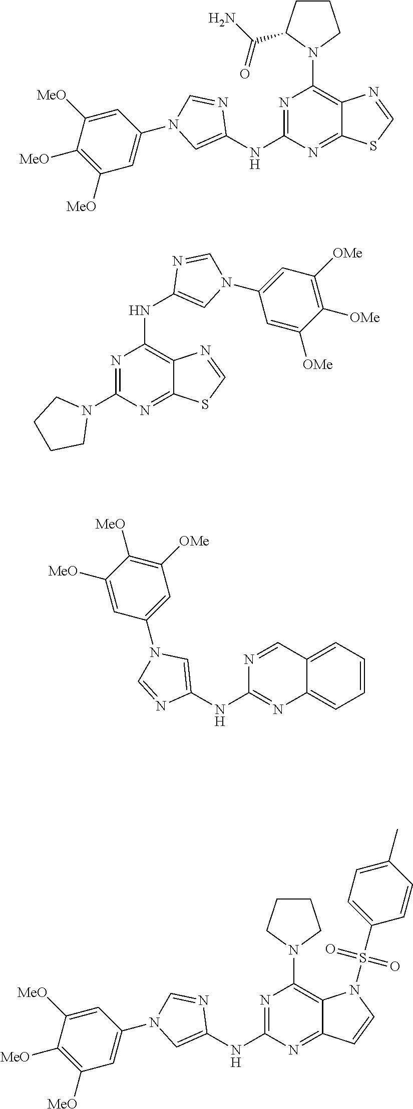 [SMILES: COc1cc(-n2cnc(Nc3nc(N4CCC[C@H]4C(N)=O)c4ncsc4n3)c2)cc(OC)c1OC ; COc1cc(-n2cnc(Nc3nc(N4CCCC4)nc4scnc34)c2)cc(OC)c1OC ; COc1cc(-n2cnc(Nc3ncc4ccccc4n3)c2)cc(OC)c1OC ; COc1cc(-n2cnc(Nc3nc(N4CCCC4)c4c(ccn4S(=O)(=O)c4ccc(C)cc4)n3)c2)cc(OC)c1OC] 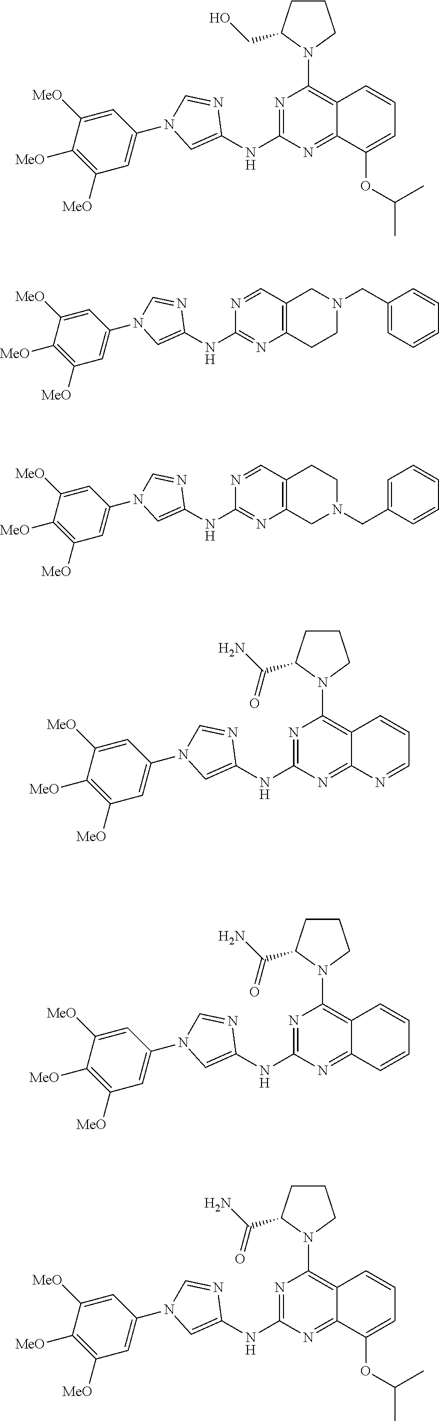 [SMILES: COc1cc(-n2cnc(Nc3nc(N4CCC[C@H]4CO)c4cccc(OC(C)C)c4n3)c2)cc(OC)c1OC ; COc1cc(-n2cnc(Nc3ncc4c(n3)CCN(Cc3ccccc3)C4)c2)cc(OC)c1OC ; COc1cc(-n2cnc(Nc3ncc4c(n3)CN(Cc3ccccc3)CC4)c2)cc(OC)c1OC ; COc1cc(-n2cnc(Nc3nc(N4CCC[C@H]4C(N)=O)c4cccnc4n3)c2)cc(OC)c1OC ; COc1cc(-n2cnc(Nc3nc(N4CCC[C@H]4C(N)=O)c4ccccc4n3)c2)cc(OC)c1OC ; COc1cc(-n2cnc(Nc3nc(N4CCC[C@H]4C(N)=O)c4cccc(OC(C)C)c4n3)c2)cc(OC)c1OC] 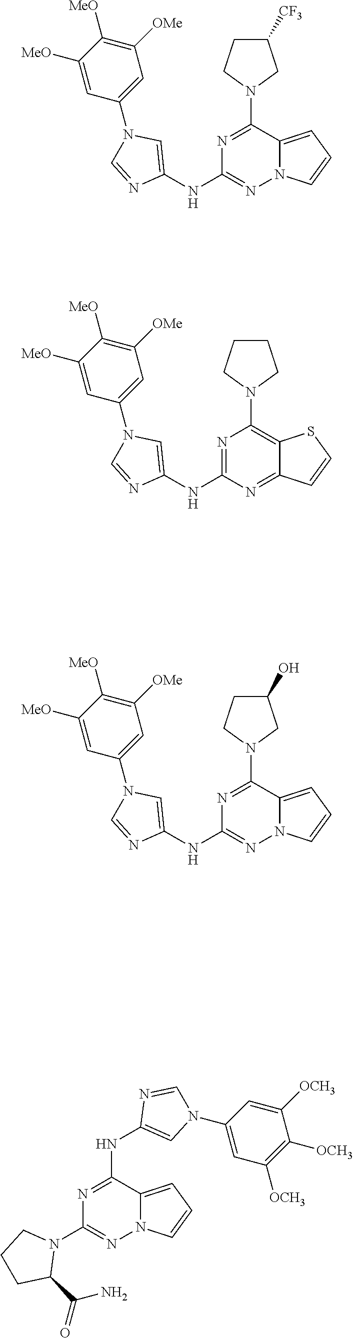 [SMILES: COc1cc(-n2cnc(Nc3nc(N4CC[C@H](C(F)(F)F)C4)c4cccn4n3)c2)cc(OC)c1OC ; COc1cc(-n2cnc(Nc3nc(N4CCCC4)c4sccc4n3)c2)cc(OC)c1OC ; COc1cc(-n2cnc(Nc3nc(N4CC[C@@H](O)C4)c4cccn4n3)c2)cc(OC)c1OC ; COc1cc(-n2cnc(Nc3nc(N4CCC[C@@H]4C(N)=O)nn4cccc34)c2)cc(OC)c1OC] 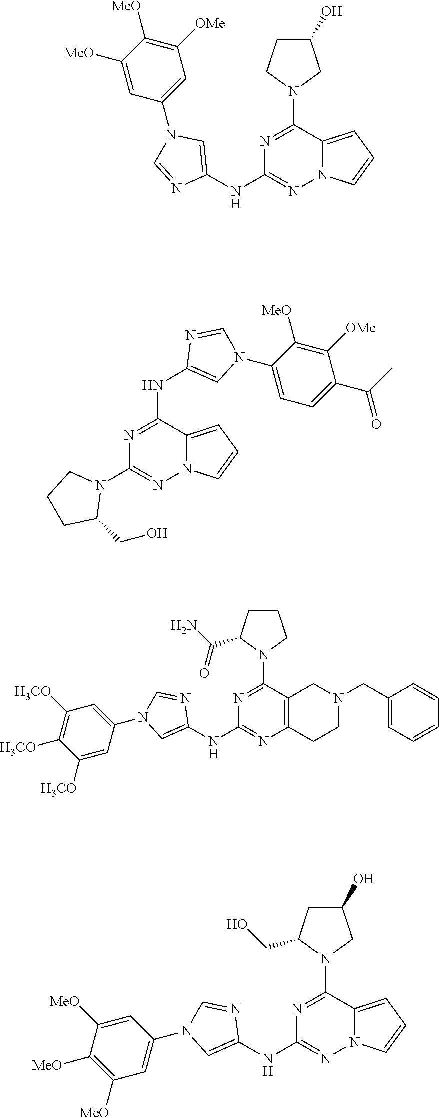 [SMILES: COc1cc(-n2cnc(Nc3nc(N4CC[C@H](O)C4)c4cccn4n3)c2)cc(OC)c1OC ; COc1c(C(C)=O)ccc(-n2cnc(Nc3nc(N4CCC[C@H]4CO)nn4cccc34)c2)c1OC ; COc1cc(-n2cnc(Nc3nc4c(c(N5CCC[C@H]5C(N)=O)n3)CN(Cc3ccccc3)CC4)c2)cc(OC)c1OC ; COc1cc(-n2cnc(Nc3nc(N4C[C@H](O)C[C@H]4CO)c4cccn4n3)c2)cc(OC)c1OC] 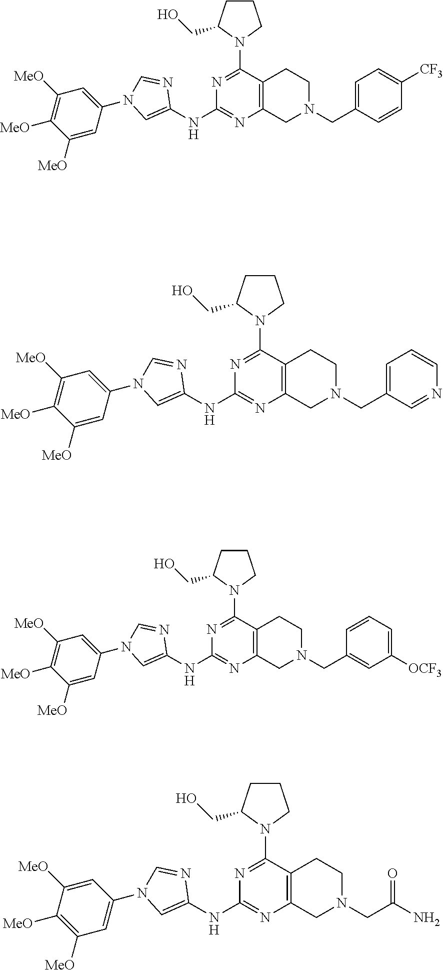 [SMILES: COc1cc(-n2cnc(Nc3nc4c(c(N5CCC[C@H]5CO)n3)CCN(Cc3ccc(C(F)(F)F)cc3)C4)c2)cc(OC)c1OC ; COc1cc(-n2cnc(Nc3nc4c(c(N5CCC[C@H]5CO)n3)CCN(Cc3cccnc3)C4)c2)cc(OC)c1OC ; COc1cc(-n2cnc(Nc3nc4c(c(N5CCC[C@H]5CO)n3)CCN(Cc3cccc(OC(F)(F)F)c3)C4)c2)cc(OC)c1OC ; COc1cc(-n2cnc(Nc3nc4c(c(N5CCC[C@H]5CO)n3)CCN(CC(N)=O)C4)c2)cc(OC)c1OC] 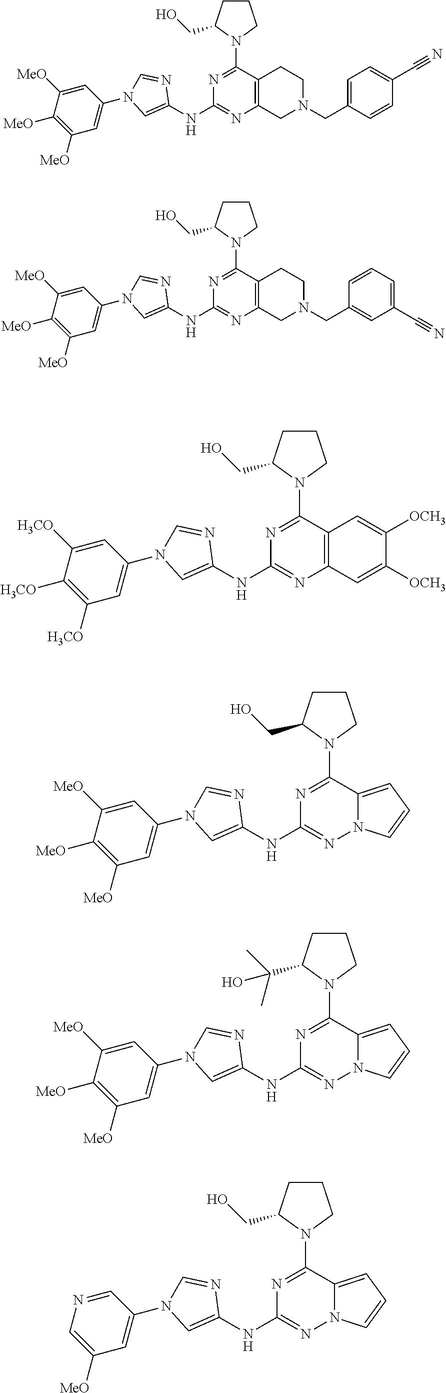 [SMILES: COc1cc(-n2cnc(Nc3nc4c(c(N5CCC[C@H]5CO)n3)CCN(Cc3ccc(C#N)cc3)C4)c2)cc(OC)c1OC ; COc1cc(-n2cnc(Nc3nc4c(c(N5CCC[C@H]5CO)n3)CCN(Cc3cccc(C#N)c3)C4)c2)cc(OC)c1OC ; COc1cc2nc(Nc3cn(-c4cc(OC)c(OC)c(OC)c4)cn3)nc(N3CCC[C@H]3CO)c2cc1OC ; COc1cc(-n2cnc(Nc3nc(N4CCC[C@@H]4CO)c4cccn4n3)c2)cc(OC)c1OC ; COc1cc(-n2cnc(Nc3nc(N4CCC[C@H]4C(C)(C)O)c4cccn4n3)c2)cc(OC)c1OC ; COc1cncc(-n2cnc(Nc3nc(N4CCC[C@H]4CO)c4cccn4n3)c2)c1] 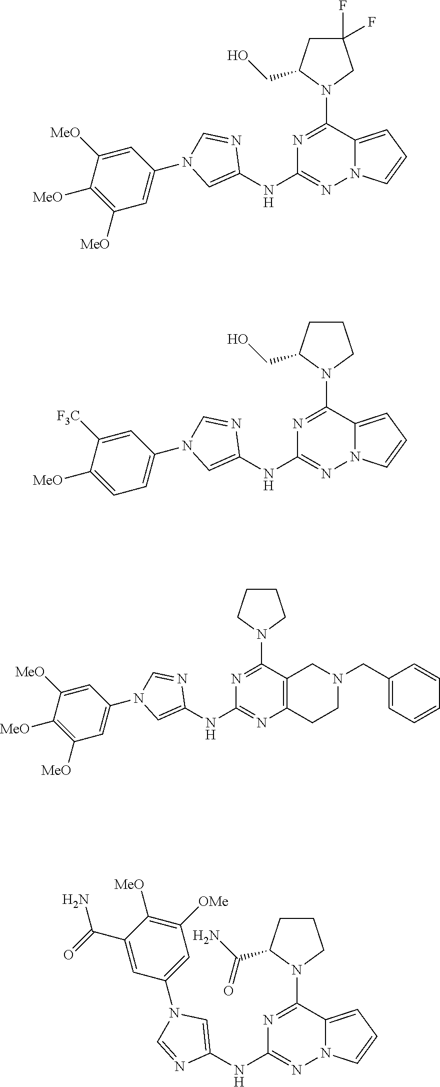 [SMILES: COc1cc(-n2cnc(Nc3nc(N4CC(F)(F)C[C@H]4CO)c4cccn4n3)c2)cc(OC)c1OC ; COc1ccc(-n2cnc(Nc3nc(N4CCC[C@H]4CO)c4cccn4n3)c2)cc1C(F)(F)F ; COc1cc(-n2cnc(Nc3nc4c(c(N5CCCC5)n3)CN(Cc3ccccc3)CC4)c2)cc(OC)c1OC ; COc1cc(-n2cnc(Nc3nc(N4CCC[C@H]4C(N)=O)c4cccn4n3)c2)cc(C(N)=O)c1OC] 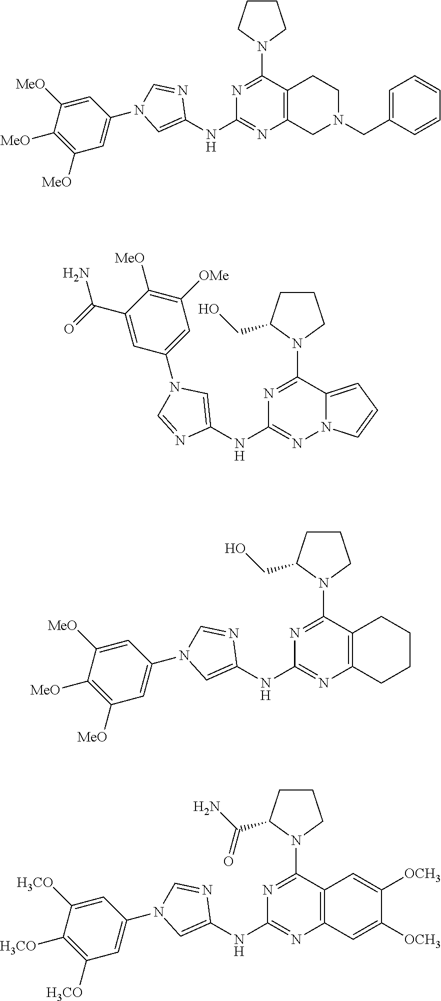 [SMILES: COc1cc(-n2cnc(Nc3nc4c(c(N5CCCC5)n3)CCN(Cc3ccccc3)C4)c2)cc(OC)c1OC ; COc1cc(-n2cnc(Nc3nc(N4CCC[C@H]4CO)c4cccn4n3)c2)cc(C(N)=O)c1OC ; COc1cc(-n2cnc(Nc3nc4c(c(N5CCC[C@H]5CO)n3)CCCC4)c2)cc(OC)c1OC ; COc1cc2nc(Nc3cn(-c4cc(OC)c(OC)c(OC)c4)cn3)nc(N3CCC[C@H]3C(N)=O)c2cc1OC] 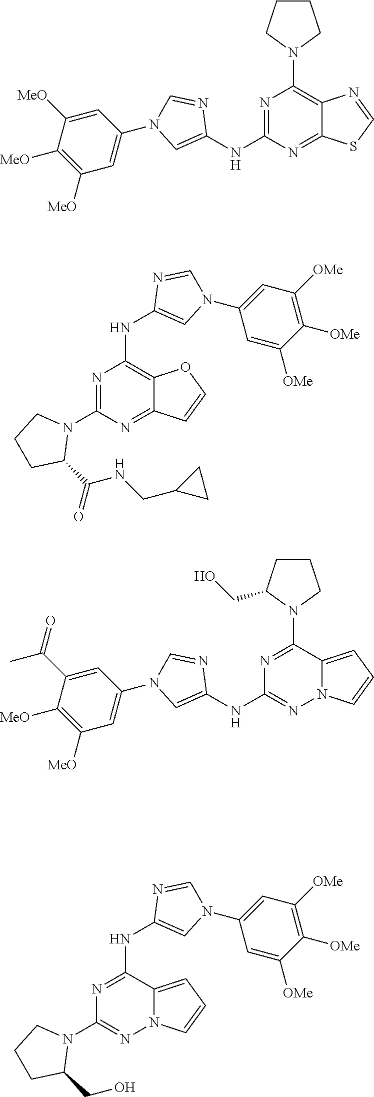 [SMILES: COc1cc(-n2cnc(Nc3nc(N4CCCC4)c4ncsc4n3)c2)cc(OC)c1OC ; COc1cc(-n2cnc(Nc3nc(N4CCC[C@H]4C(=O)NCC4CC4)nc4ccoc34)c2)cc(OC)c1OC ; COc1cc(-n2cnc(Nc3nc(N4CCC[C@H]4CO)c4cccn4n3)c2)cc(C(C)=O)c1OC ; COc1cc(-n2cnc(Nc3nc(N4CCC[C@@H]4CO)nn4cccc34)c2)cc(OC)c1OC] 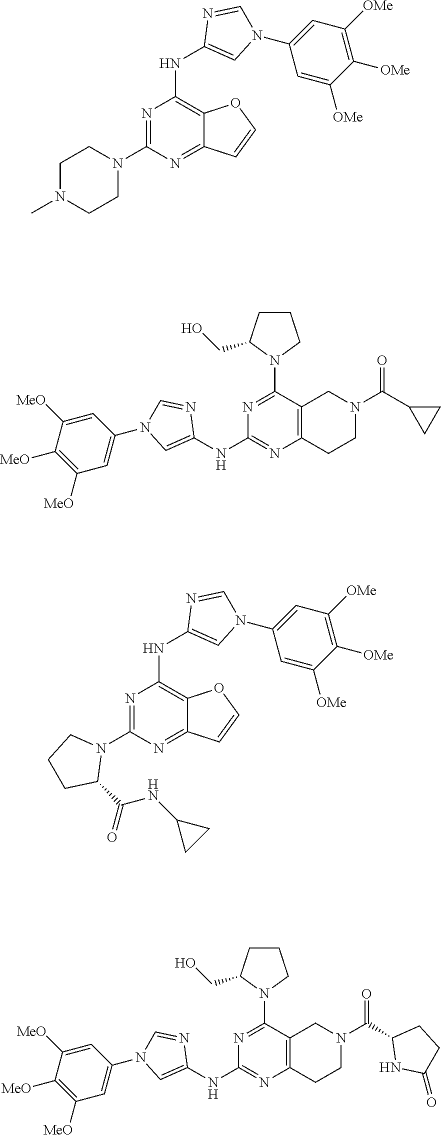 [SMILES: COc1cc(-n2cnc(Nc3nc(N4CCN(C)CC4)nc4ccoc34)c2)cc(OC)c1OC ; COc1cc(-n2cnc(Nc3nc4c(c(N5CCC[C@H]5CO)n3)CN(C(=O)C3CC3)CC4)c2)cc(OC)c1OC ; COc1cc(-n2cnc(Nc3nc(N4CCC[C@H]4C(=O)NC4CC4)nc4ccoc34)c2)cc(OC)c1OC ; COc1cc(-n2cnc(Nc3nc4c(c(N5CCC[C@H]5CO)n3)CN(C(=O)[C@@H]3CCC(=O)N3)CC4)c2)cc(OC)c1OC] 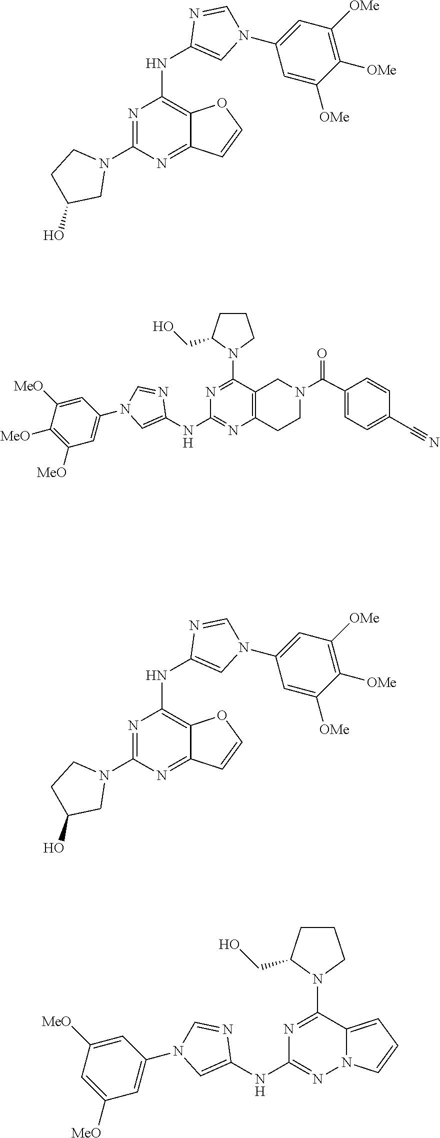 [SMILES: COc1cc(-n2cnc(Nc3nc(N4CC[C@@H](O)C4)nc4ccoc34)c2)cc(OC)c1OC ; COc1cc(-n2cnc(Nc3nc4c(c(N5CCC[C@H]5CO)n3)CN(C(=O)c3ccc(C#N)cc3)CC4)c2)cc(OC)c1OC ; COc1cc(-n2cnc(Nc3nc(N4CC[C@H](O)C4)nc4ccoc34)c2)cc(OC)c1OC ; COc1cc(OC)cc(-n2cnc(Nc3nc(N4CCC[C@H]4CO)c4cccn4n3)c2)c1] 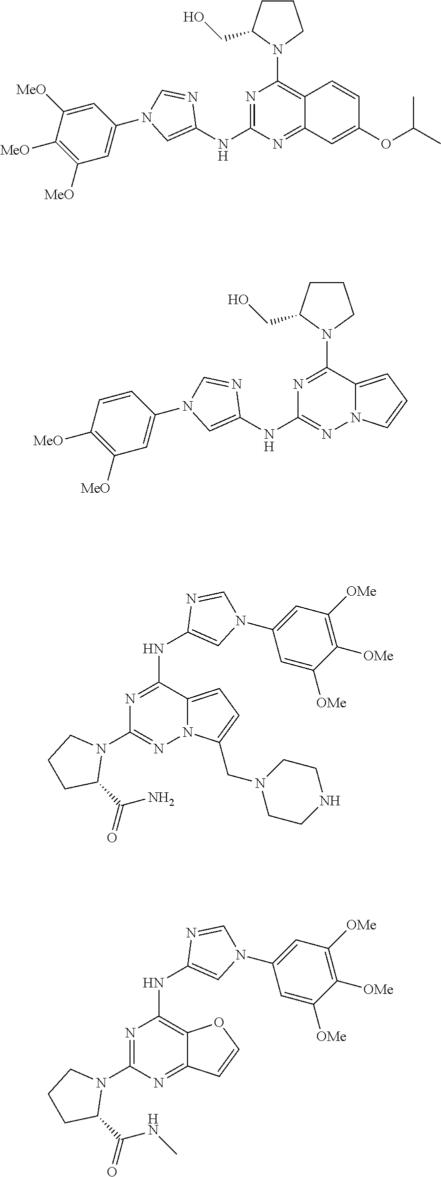 [SMILES: COc1cc(-n2cnc(Nc3nc(N4CCC[C@H]4CO)c4ccc(OC(C)C)cc4n3)c2)cc(OC)c1OC ; COc1ccc(-n2cnc(Nc3nc(N4CCC[C@H]4CO)c4cccn4n3)c2)cc1OC ; COc1cc(-n2cnc(Nc3nc(N4CCC[C@H]4C(N)=O)nn4c(CN5CCNCC5)ccc34)c2)cc(OC)c1OC ; CNC(=O)[C@@H]1CCCN1c1nc(Nc2cn(-c3cc(OC)c(OC)c(OC)c3)cn2)c2occc2n1] 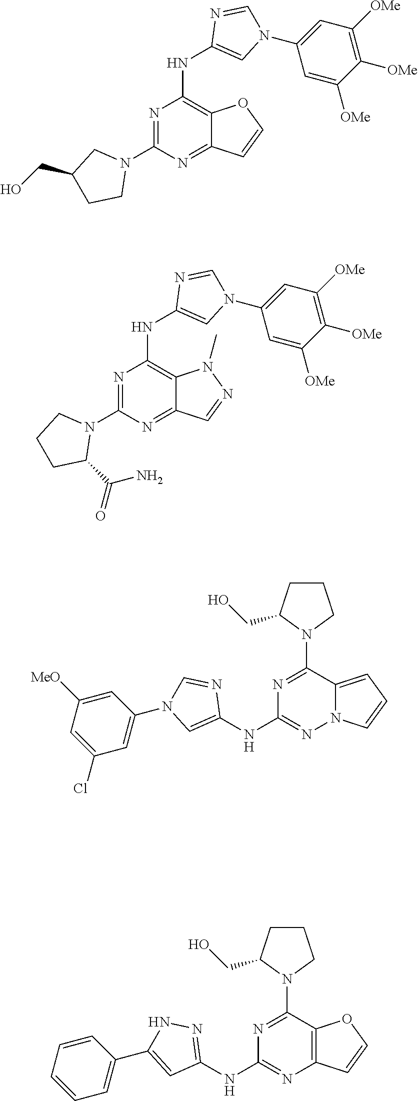 [SMILES: COc1cc(-n2cnc(Nc3nc(N4CC[C@@H](CO)C4)nc4ccoc34)c2)cc(OC)c1OC ; COc1cc(-n2cnc(Nc3nc(N4CCC[C@H]4C(N)=O)nc4cnn(C)c34)c2)cc(OC)c1OC ; COc1cc(Cl)cc(-n2cnc(Nc3nc(N4CCC[C@H]4CO)c4cccn4n3)c2)c1 ; OC[C@@H]1CCCN1c1nc(Nc2cc(-c3ccccc3)[nH]n2)nc2ccoc12] 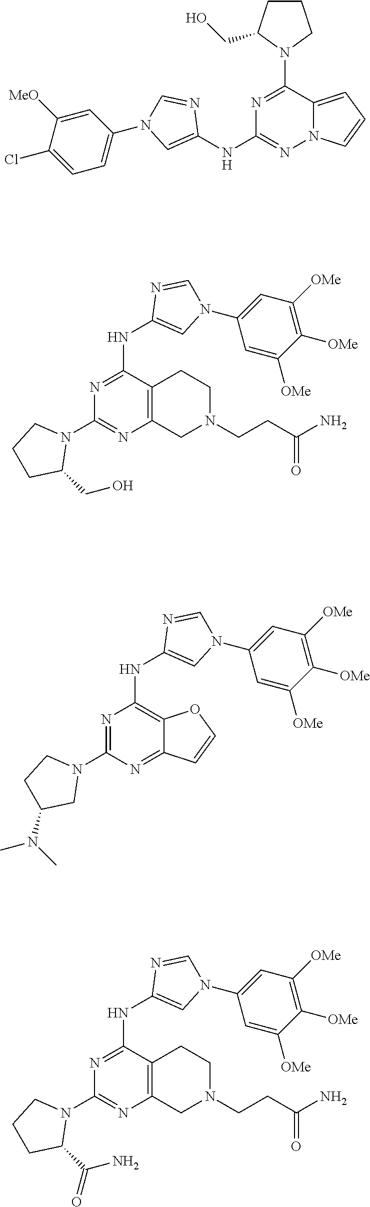 [SMILES: COc1cc(-n2cnc(Nc3nc(N4CCC[C@H]4CO)c4cccn4n3)c2)ccc1Cl ; COc1cc(-n2cnc(Nc3nc(N4CCC[C@H]4CO)nc4c3CCN(CCC(N)=O)C4)c2)cc(OC)c1OC ; COc1cc(-n2cnc(Nc3nc(N4CC[C@@H](N(C)C)C4)nc4ccoc34)c2)cc(OC)c1OC ; COc1cc(-n2cnc(Nc3nc(N4CCC[C@H]4C(N)=O)nc4c3CCN(CCC(N)=O)C4)c2)cc(OC)c1OC] 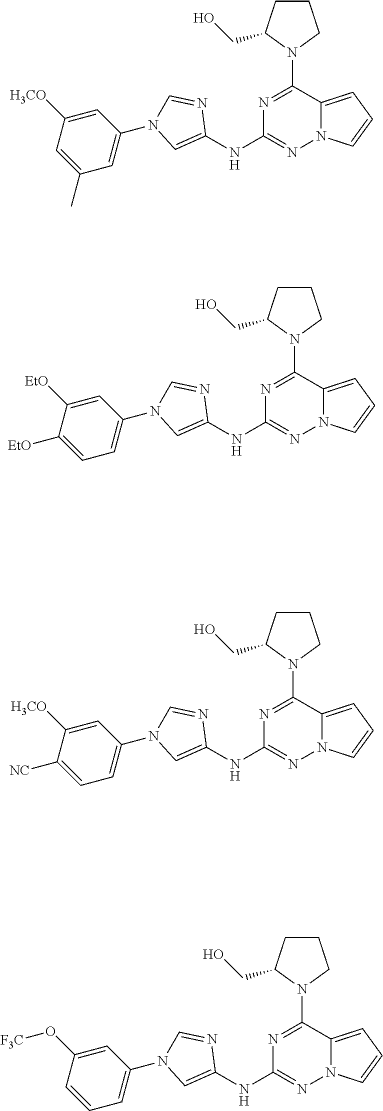 [SMILES: COc1cc(C)cc(-n2cnc(Nc3nc(N4CCC[C@H]4CO)c4cccn4n3)c2)c1 ; CCOc1ccc(-n2cnc(Nc3nc(N4CCC[C@H]4CO)c4cccn4n3)c2)cc1OCC ; COc1cc(-n2cnc(Nc3nc(N4CCC[C@H]4CO)c4cccn4n3)c2)ccc1C#N ; OC[C@@H]1CCCN1c1nc(Nc2cn(-c3cccc(OC(F)(F)F)c3)cn2)nn2cccc12] 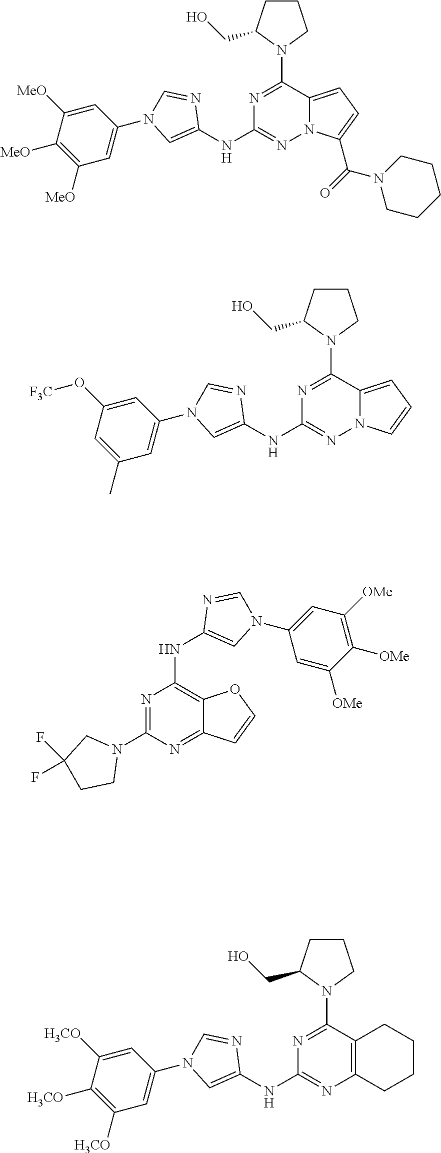 [SMILES: COc1cc(-n2cnc(Nc3nc(N4CCC[C@H]4CO)c4ccc(C(=O)N5CCCCC5)n4n3)c2)cc(OC)c1OC ; Cc1cc(OC(F)(F)F)cc(-n2cnc(Nc3nc(N4CCC[C@H]4CO)c4cccn4n3)c2)c1 ; COc1cc(-n2cnc(Nc3nc(N4CCC(F)(F)C4)nc4ccoc34)c2)cc(OC)c1OC ; COc1cc(-n2cnc(Nc3nc4c(c(N5CCC[C@@H]5CO)n3)CCCC4)c2)cc(OC)c1OC] 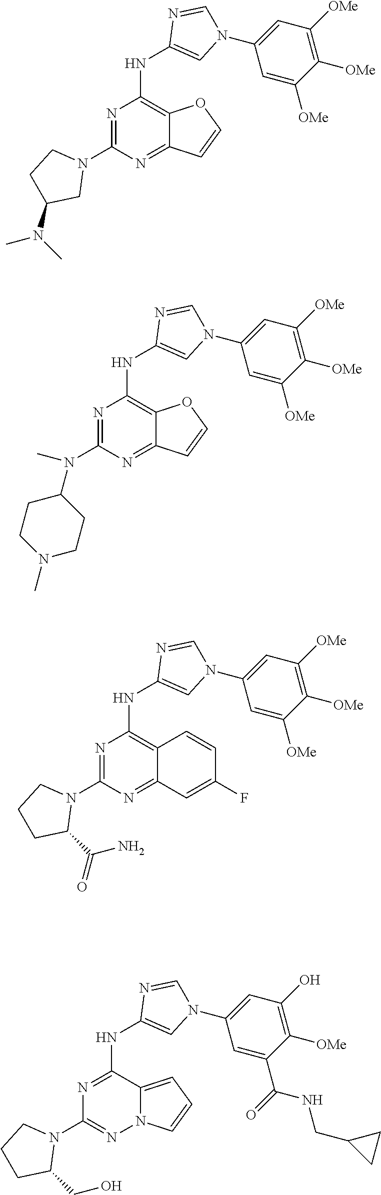 [SMILES: COc1cc(-n2cnc(Nc3nc(N4CC[C@H](N(C)C)C4)nc4ccoc34)c2)cc(OC)c1OC ; COc1cc(-n2cnc(Nc3nc(N(C)C4CCN(C)CC4)nc4ccoc34)c2)cc(OC)c1OC ; COc1cc(-n2cnc(Nc3nc(N4CCC[C@H]4C(N)=O)nc4cc(F)ccc34)c2)cc(OC)c1OC ; COc1c(O)cc(-n2cnc(Nc3nc(N4CCC[C@H]4CO)nn4cccc34)c2)cc1C(=O)NCC1CC1] 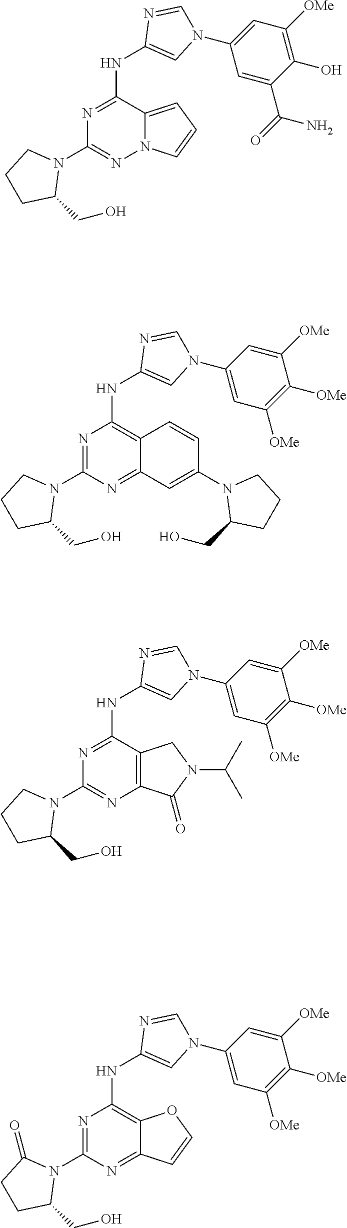 [SMILES: COc1cc(-n2cnc(Nc3nc(N4CCC[C@H]4CO)nn4cccc34)c2)cc(C(N)=O)c1O ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4CO)nc4c3CN(C(C)C)C4=O)c2)cc(OC)c1OC ; COc1cc(-n2cnc(Nc3nc(N4C(=O)CC[C@H]4CO)nc4ccoc34)c2)cc(OC)c1OC] 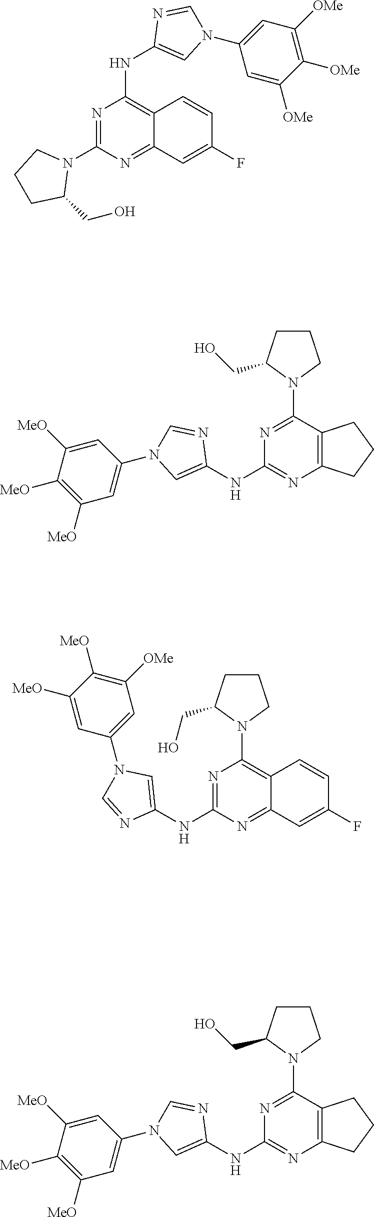 [SMILES: COc1cc(-n2cnc(Nc3nc(N4CCC[C@H]4CO)nc4cc(F)ccc34)c2)cc(OC)c1OC ; COc1cc(-n2cnc(Nc3nc4c(c(N5CCC[C@H]5CO)n3)CCC4)c2)cc(OC)c1OC ; COc1cc(-n2cnc(Nc3nc(N4CCC[C@H]4CO)c4ccc(F)cc4n3)c2)cc(OC)c1OC ; COc1cc(-n2cnc(Nc3nc4c(c(N5CCC[C@@H]5CO)n3)CCC4)c2)cc(OC)c1OC] 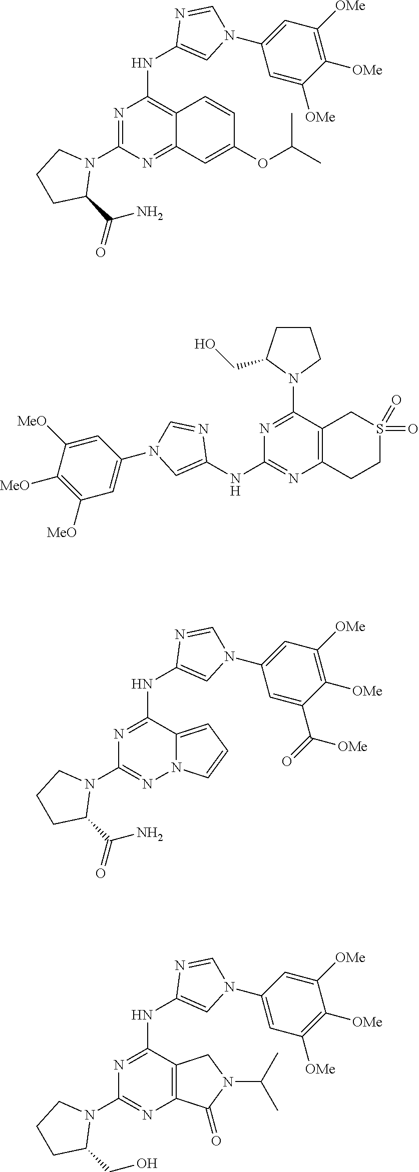 [SMILES: COc1cc(-n2cnc(Nc3nc(N4CCC[C@@H]4C(N)=O)nc4cc(OC(C)C)ccc34)c2)cc(OC)c1OC ; COc1cc(-n2cnc(Nc3nc4c(c(N5CCC[C@H]5CO)n3)CS(=O)(=O)CC4)c2)cc(OC)c1OC ; COC(=O)c1cc(-n2cnc(Nc3nc(N4CCC[C@H]4C(N)=O)nn4cccc34)c2)cc(OC)c1OC ; COc1cc(-n2cnc(Nc3nc(N4CCC[C@H]4CO)nc4c3CN(C(C)C)C4=O)c2)cc(OC)c1OC] 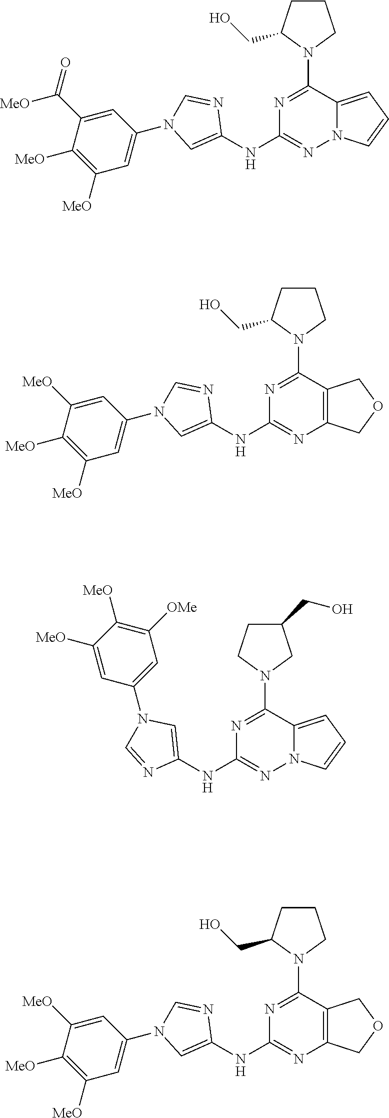 [SMILES: COC(=O)c1cc(-n2cnc(Nc3nc(N4CCC[C@H]4CO)c4cccn4n3)c2)cc(OC)c1OC ; COc1cc(-n2cnc(Nc3nc4c(c(N5CCC[C@H]5CO)n3)COC4)c2)cc(OC)c1OC ; COc1cc(-n2cnc(Nc3nc(N4CC[C@@H](CO)C4)c4cccn4n3)c2)cc(OC)c1OC ; COc1cc(-n2cnc(Nc3nc4c(c(N5CCC[C@@H]5CO)n3)COC4)c2)cc(OC)c1OC] 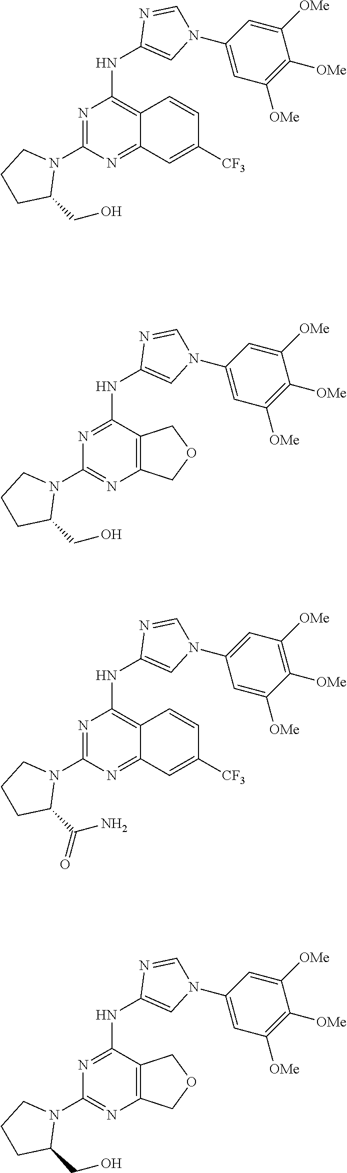 [SMILES: COc1cc(-n2cnc(Nc3nc(N4CCC[C@H]4CO)nc4cc(C(F)(F)F)ccc34)c2)cc(OC)c1OC ; COc1cc(-n2cnc(Nc3nc(N4CCC[C@H]4CO)nc4c3COC4)c2)cc(OC)c1OC ; COc1cc(-n2cnc(Nc3nc(N4CCC[C@H]4C(N)=O)nc4cc(C(F)(F)F)ccc34)c2)cc(OC)c1OC ; COc1cc(-n2cnc(Nc3nc(N4CCC[C@@H]4CO)nc4c3COC4)c2)cc(OC)c1OC] 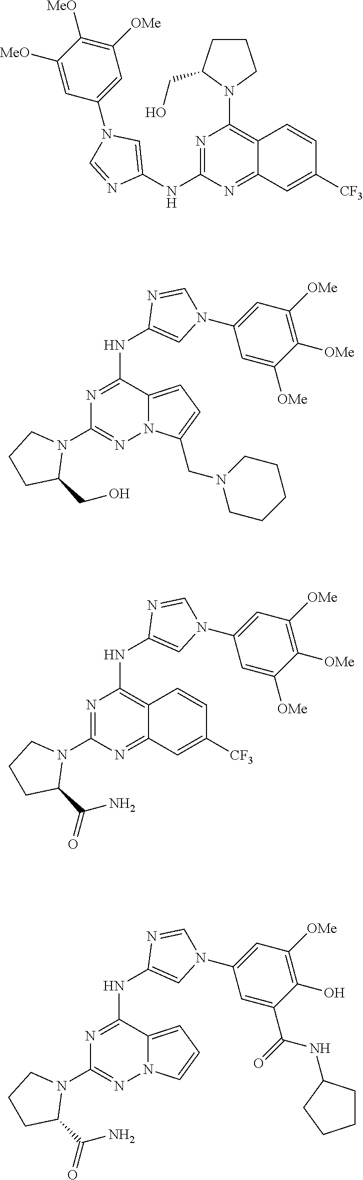 [SMILES: COc1cc(-n2cnc(Nc3nc(N4CCC[C@H]4CO)c4ccc(C(F)(F)F)cc4n3)c2)cc(OC)c1OC ; COc1cc(-n2cnc(Nc3nc(N4CCC[C@@H]4CO)nn4c(CN5CCCCC5)ccc34)c2)cc(OC)c1OC ; COc1cc(-n2cnc(Nc3nc(N4CCC[C@@H]4C(N)=O)nc4cc(C(F)(F)F)ccc34)c2)cc(OC)c1OC ; COc1cc(-n2cnc(Nc3nc(N4CCC[C@H]4C(N)=O)nn4cccc34)c2)cc(C(=O)NC2CCCC2)c1O] 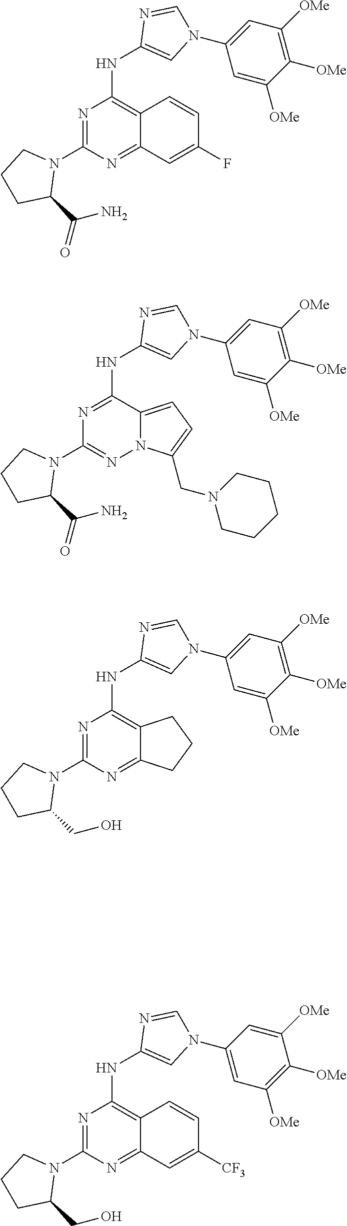 [SMILES: COc1cc(-n2cnc(Nc3nc(N4CCC[C@@H]4C(N)=O)nc4cc(F)ccc34)c2)cc(OC)c1OC ; COc1cc(-n2cnc(Nc3nc(N4CCC[C@@H]4C(N)=O)nn4c(CN5CCCCC5)ccc34)c2)cc(OC)c1OC ; COc1cc(-n2cnc(Nc3nc(N4CCC[C@H]4CO)nc4c3CCC4)c2)cc(OC)c1OC ; COc1cc(-n2cnc(Nc3nc(N4CCC[C@@H]4CO)nc4cc(C(F)(F)F)ccc34)c2)cc(OC)c1OC] 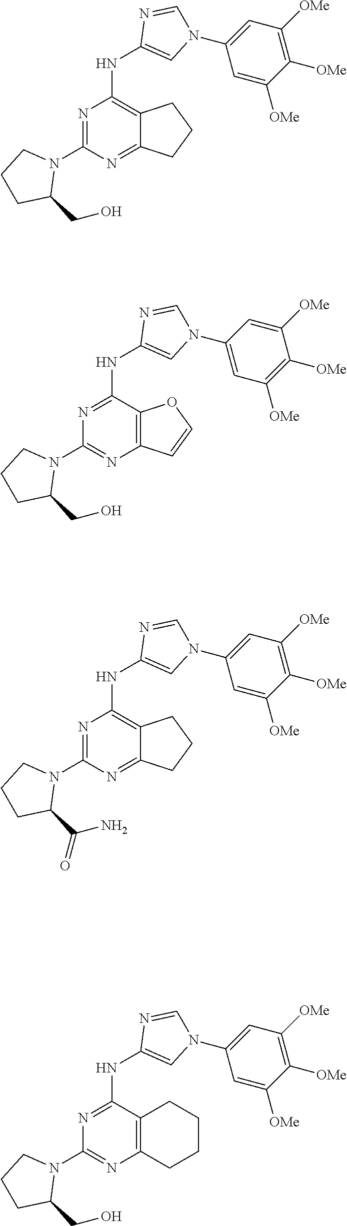 [SMILES: COc1cc(-n2cnc(Nc3nc(N4CCC[C@@H]4CO)nc4c3CCC4)c2)cc(OC)c1OC ; COc1cc(-n2cnc(Nc3nc(N4CCC[C@@H]4CO)nc4ccoc34)c2)cc(OC)c1OC ; COc1cc(-n2cnc(Nc3nc(N4CCC[C@@H]4C(N)=O)nc4c3CCC4)c2)cc(OC)c1OC ; COc1cc(-n2cnc(Nc3nc(N4CCC[C@@H]4CO)nc4c3CCCC4)c2)cc(OC)c1OC] 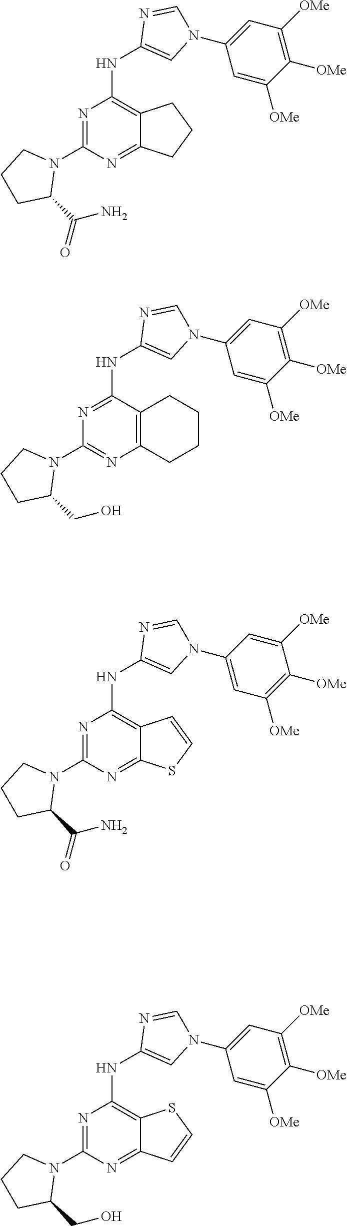 [SMILES: COc1cc(-n2cnc(Nc3nc(N4CCC[C@H]4C(N)=O)nc4c3CCC4)c2)cc(OC)c1OC ; COc1cc(-n2cnc(Nc3nc(N4CCC[C@H]4CO)nc4c3CCCC4)c2)cc(OC)c1OC ; COc1cc(-n2cnc(Nc3nc(N4CCC[C@@H]4C(N)=O)nc4sccc34)c2)cc(OC)c1OC ; COc1cc(-n2cnc(Nc3nc(N4CCC[C@@H]4CO)nc4ccsc34)c2)cc(OC)c1OC] 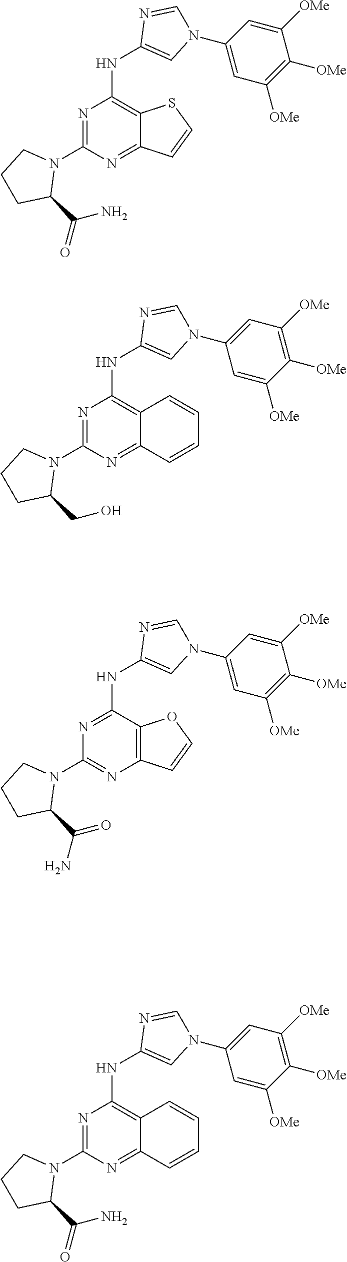 [SMILES: COc1cc(-n2cnc(Nc3nc(N4CCC[C@@H]4C(N)=O)nc4ccsc34)c2)cc(OC)c1OC ; COc1cc(-n2cnc(Nc3nc(N4CCC[C@@H]4CO)nc4ccccc34)c2)cc(OC)c1OC ; COc1cc(-n2cnc(Nc3nc(N4CCC[C@@H]4C(N)=O)nc4ccoc34)c2)cc(OC)c1OC ; COc1cc(-n2cnc(Nc3nc(N4CCC[C@@H]4C(N)=O)nc4ccccc34)c2)cc(OC)c1OC] 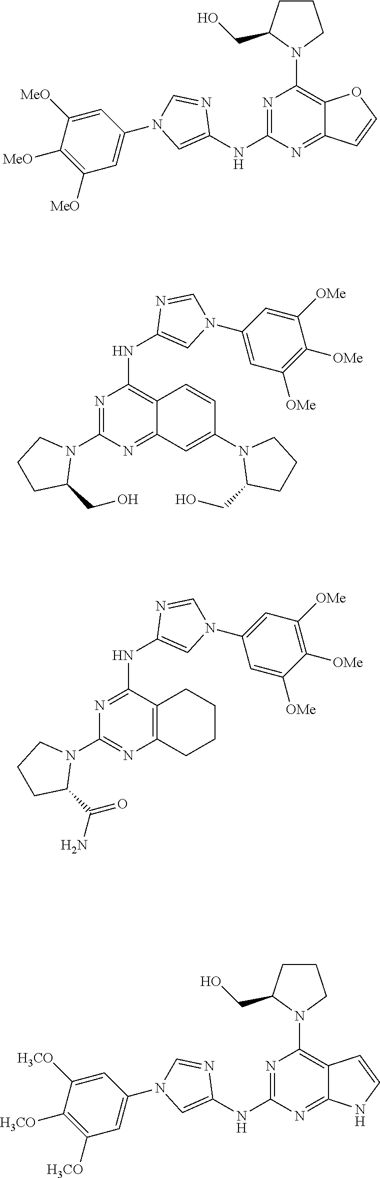 [SMILES: COc1cc(-n2cnc(Nc3nc(N4CCC[C@@H]4CO)c4occc4n3)c2)cc(OC)c1OC ; COc1cc(-n2cnc(Nc3nc(N4CCC[C@@H]4CO)nc4cc(N5CCC[C@@H]5CO)ccc34)c2)cc(OC)c1OC ; COc1cc(-n2cnc(Nc3nc(N4CCC[C@H]4C(N)=O)nc4c3CCCC4)c2)cc(OC)c1OC ; COc1cc(-n2cnc(Nc3nc(N4CCC[C@@H]4CO)c4cc[nH]c4n3)c2)cc(OC)c1OC] 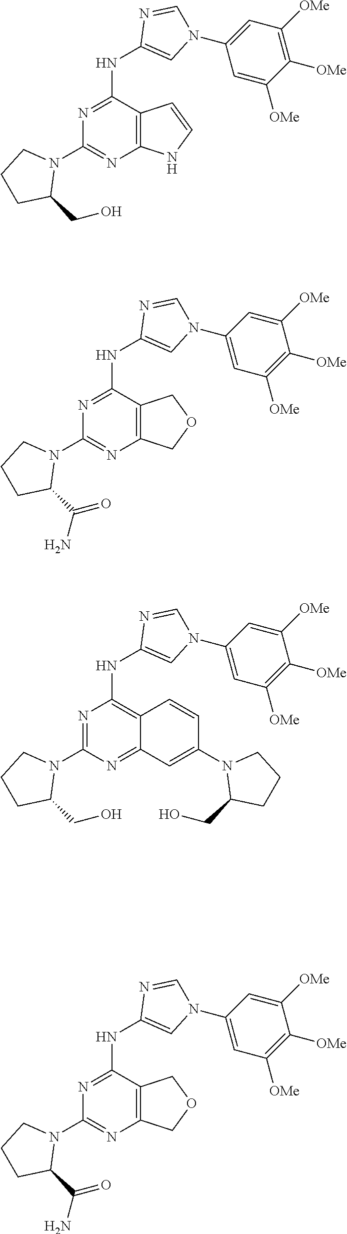 [SMILES: COc1cc(-n2cnc(Nc3nc(N4CCC[C@@H]4CO)nc4[nH]ccc34)c2)cc(OC)c1OC ; COc1cc(-n2cnc(Nc3nc(N4CCC[C@H]4C(N)=O)nc4c3COC4)c2)cc(OC)c1OC ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4C(N)=O)nc4c3COC4)c2)cc(OC)c1OC] 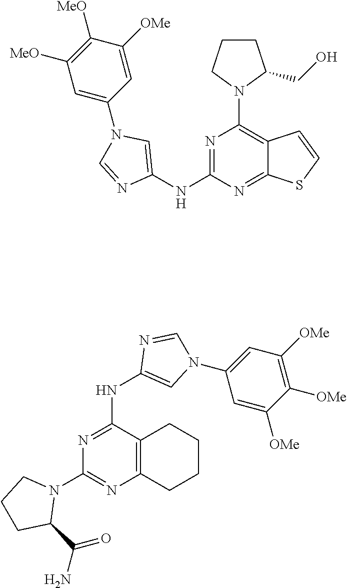 [SMILES: COc1cc(-n2cnc(Nc3nc(N4CCC[C@@H]4CO)c4ccsc4n3)c2)cc(OC)c1OC ; COc1cc(-n2cnc(Nc3nc(N4CCC[C@@H]4C(N)=O)nc4c3CCCC4)c2)cc(OC)c1OC] 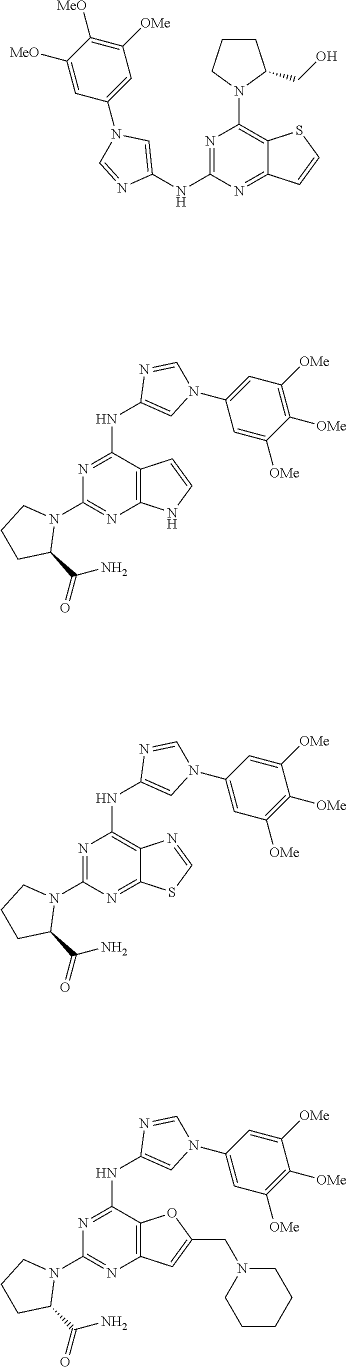 [SMILES: COc1cc(-n2cnc(Nc3nc(N4CCC[C@@H]4CO)c4sccc4n3)c2)cc(OC)c1OC ; COc1cc(-n2cnc(Nc3nc(N4CCC[C@@H]4C(N)=O)nc4[nH]ccc34)c2)cc(OC)c1OC ; COc1cc(-n2cnc(Nc3nc(N4CCC[C@@H]4C(N)=O)nc4scnc34)c2)cc(OC)c1OC ; COc1cc(-n2cnc(Nc3nc(N4CCC[C@H]4C(N)=O)nc4cc(CN5CCCCC5)oc34)c2)cc(OC)c1OC] 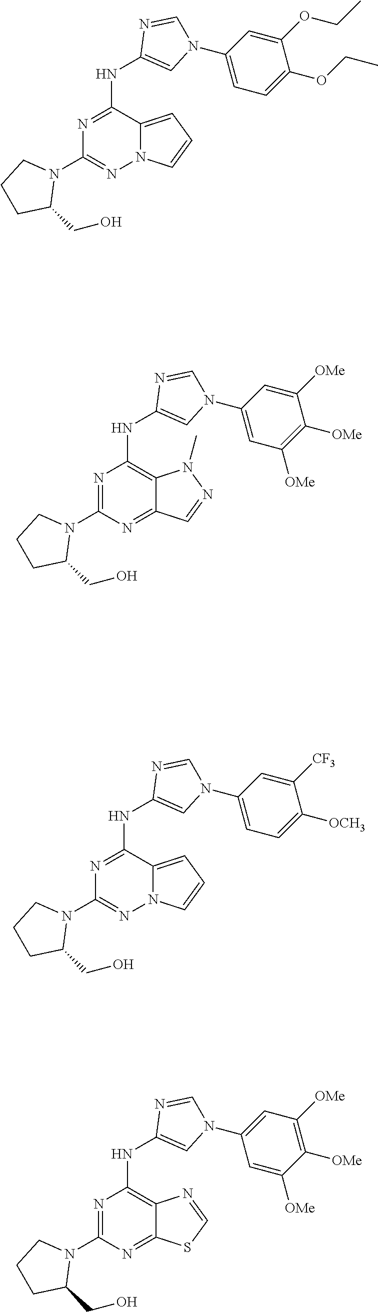 [SMILES: CCOc1ccc(-n2cnc(Nc3nc(N4CCC[C@H]4CO)nn4cccc34)c2)cc1OCC ; COc1cc(-n2cnc(Nc3nc(N4CCC[C@H]4CO)nc4cnn(C)c34)c2)cc(OC)c1OC ; COc1ccc(-n2cnc(Nc3nc(N4CCC[C@H]4CO)nn4cccc34)c2)cc1C(F)(F)F ; COc1cc(-n2cnc(Nc3nc(N4CCC[C@@H]4CO)nc4scnc34)c2)cc(OC)c1OC] 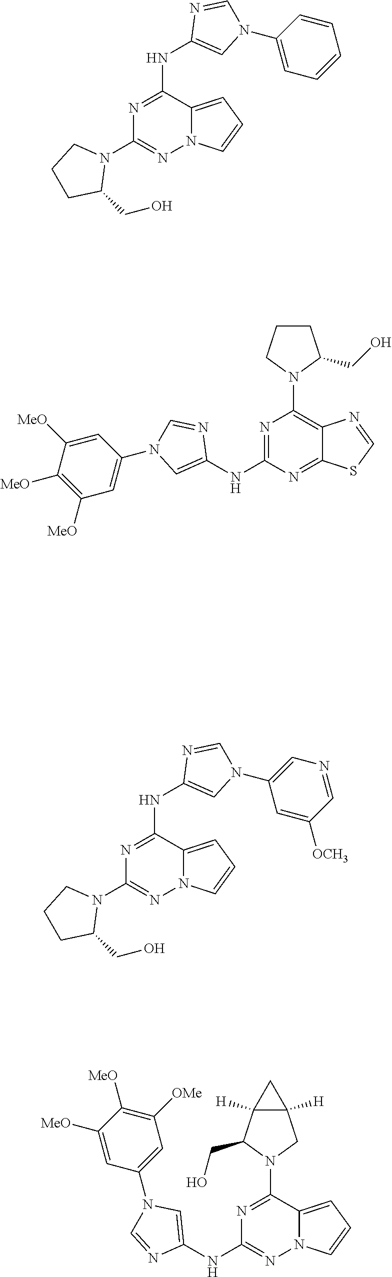 [SMILES: OC[C@@H]1CCCN1c1nc(Nc2cn(-c3ccccc3)cn2)c2cccn2n1 ; COc1cc(-n2cnc(Nc3nc(N4CCC[C@@H]4CO)c4ncsc4n3)c2)cc(OC)c1OC ; COc1cncc(-n2cnc(Nc3nc(N4CCC[C@H]4CO)nn4cccc34)c2)c1 ; COc1cc(-n2cnc(Nc3nc(N4C[C@@H]5C[C@@H]5[C@@H]4CO)c4cccn4n3)c2)cc(OC)c1OC] 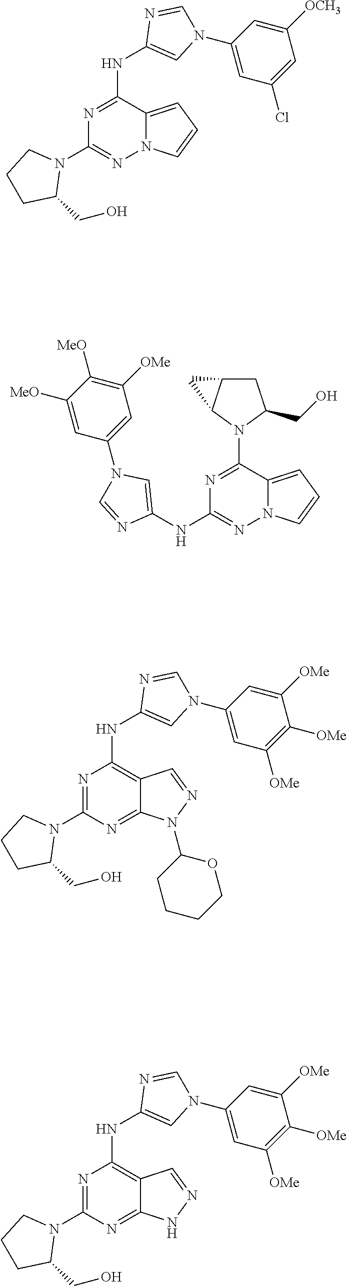 [SMILES: COc1cc(Cl)cc(-n2cnc(Nc3nc(N4CCC[C@H]4CO)nn4cccc34)c2)c1 ; COc1cc(-n2cnc(Nc3nc(N4[C@H](CO)C[C@H]5C[C@H]54)c4cccn4n3)c2)cc(OC)c1OC ; COc1cc(-n2cnc(Nc3nc(N4CCC[C@H]4CO)nc4c3cnn4C3CCCCO3)c2)cc(OC)c1OC ; COc1cc(-n2cnc(Nc3nc(N4CCC[C@H]4CO)nc4[nH]ncc34)c2)cc(OC)c1OC] 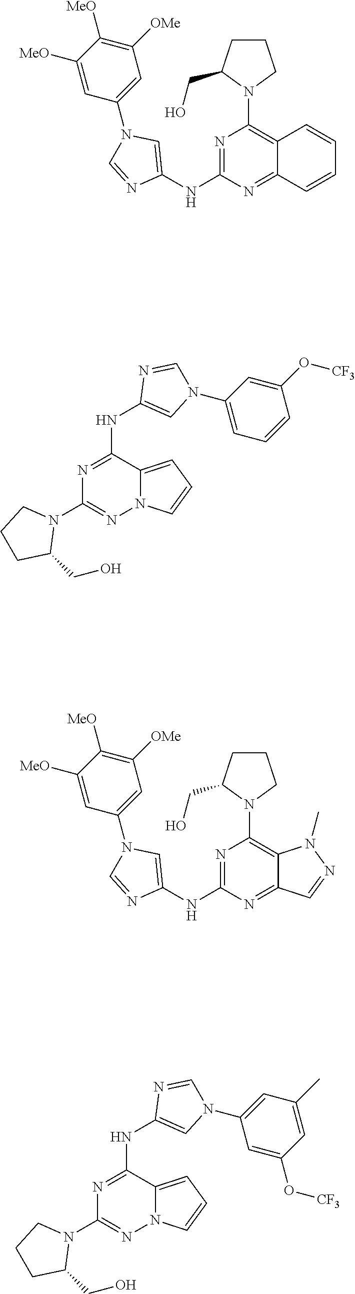 [SMILES: COc1cc(-n2cnc(Nc3nc(N4CCC[C@@H]4CO)c4ccccc4n3)c2)cc(OC)c1OC ; OC[C@@H]1CCCN1c1nc(Nc2cn(-c3cccc(OC(F)(F)F)c3)cn2)c2cccn2n1 ; COc1cc(-n2cnc(Nc3nc(N4CCC[C@H]4CO)c4c(cnn4C)n3)c2)cc(OC)c1OC ; Cc1cc(OC(F)(F)F)cc(-n2cnc(Nc3nc(N4CCC[C@H]4CO)nn4cccc34)c2)c1] 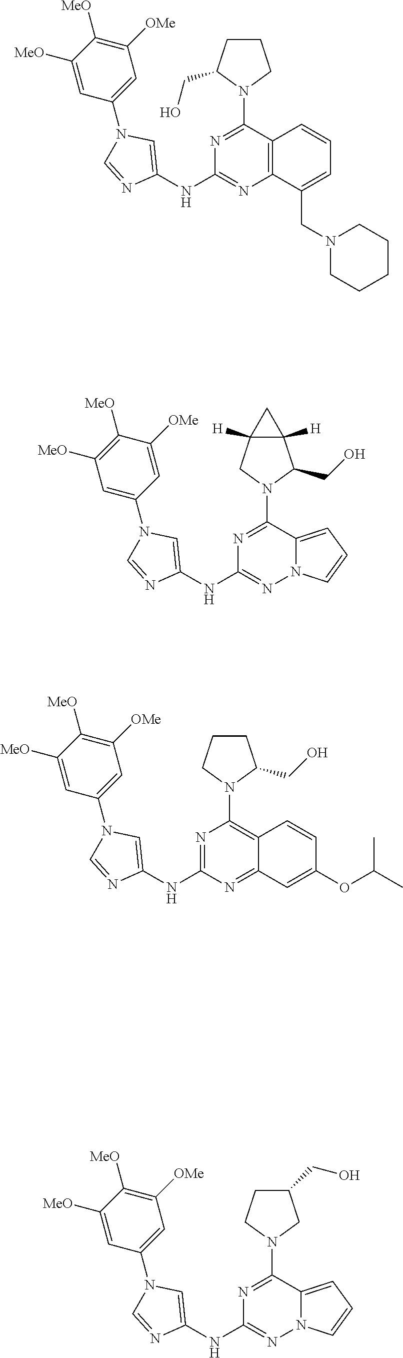 [SMILES: COc1cc(-n2cnc(Nc3nc(N4CCC[C@H]4CO)c4cccc(CN5CCCCC5)c4n3)c2)cc(OC)c1OC ; COc1cc(-n2cnc(Nc3nc(N4C[C@@H]5C[C@@H]5[C@H]4CO)c4cccn4n3)c2)cc(OC)c1OC ; COc1cc(-n2cnc(Nc3nc(N4CCC[C@@H]4CO)c4ccc(OC(C)C)cc4n3)c2)cc(OC)c1OC ; COc1cc(-n2cnc(Nc3nc(N4CC[C@H](CO)C4)c4cccn4n3)c2)cc(OC)c1OC] 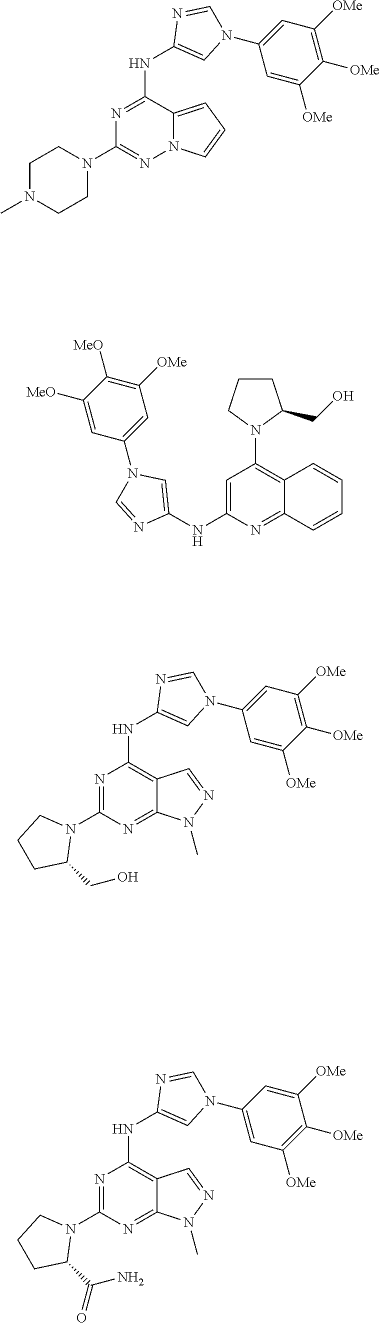 [SMILES: COc1cc(-n2cnc(Nc3nc(N4CCN(C)CC4)nn4cccc34)c2)cc(OC)c1OC ; COc1cc(-n2cnc(Nc3cc(N4CCC[C@H]4CO)c4ccccc4n3)c2)cc(OC)c1OC ; COc1cc(-n2cnc(Nc3nc(N4CCC[C@H]4CO)nc4c3cnn4C)c2)cc(OC)c1OC ; COc1cc(-n2cnc(Nc3nc(N4CCC[C@H]4C(N)=O)nc4c3cnn4C)c2)cc(OC)c1OC] 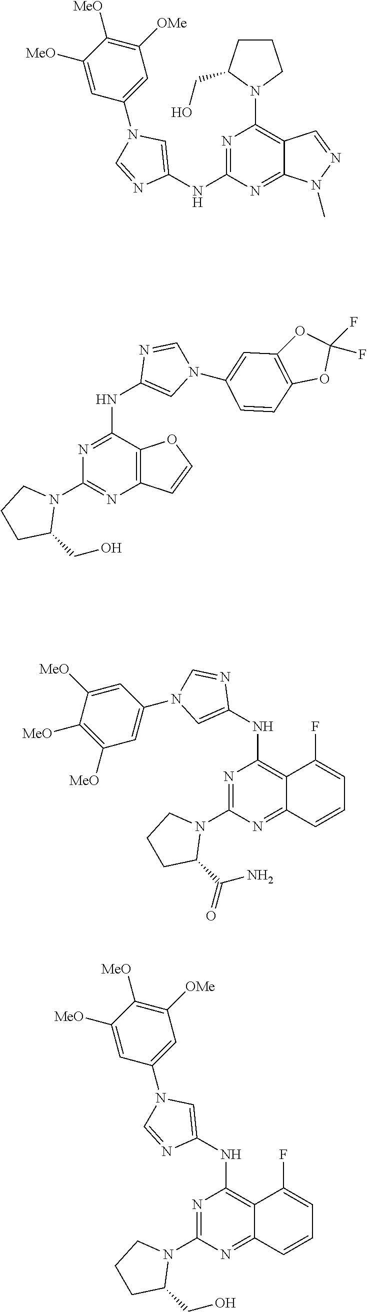 [SMILES: COc1cc(-n2cnc(Nc3nc(N4CCC[C@H]4CO)c4cnn(C)c4n3)c2)cc(OC)c1OC ; OC[C@@H]1CCCN1c1nc(Nc2cn(-c3ccc4c(c3)OC(F)(F)O4)cn2)c2occc2n1 ; COc1cc(-n2cnc(Nc3nc(N4CCC[C@H]4C(N)=O)nc4cccc(F)c34)c2)cc(OC)c1OC ; COc1cc(-n2cnc(Nc3nc(N4CCC[C@H]4CO)nc4cccc(F)c34)c2)cc(OC)c1OC] 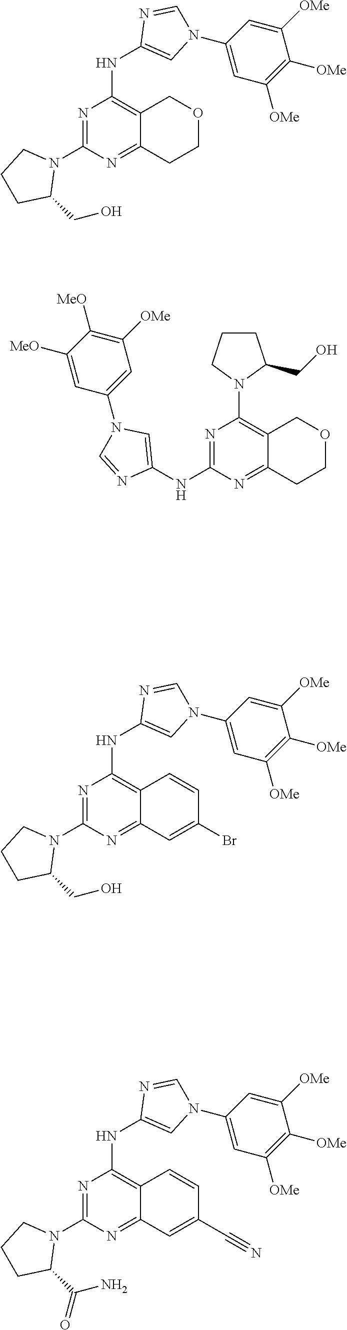 [SMILES: COc1cc(-n2cnc(Nc3nc(N4CCC[C@H]4CO)nc4c3COCC4)c2)cc(OC)c1OC ; COc1cc(-n2cnc(Nc3nc4c(c(N5CCC[C@H]5CO)n3)COCC4)c2)cc(OC)c1OC ; COc1cc(-n2cnc(Nc3nc(N4CCC[C@H]4CO)nc4cc(Br)ccc34)c2)cc(OC)c1OC ; COc1cc(-n2cnc(Nc3nc(N4CCC[C@H]4C(N)=O)nc4cc(C#N)ccc34)c2)cc(OC)c1OC] 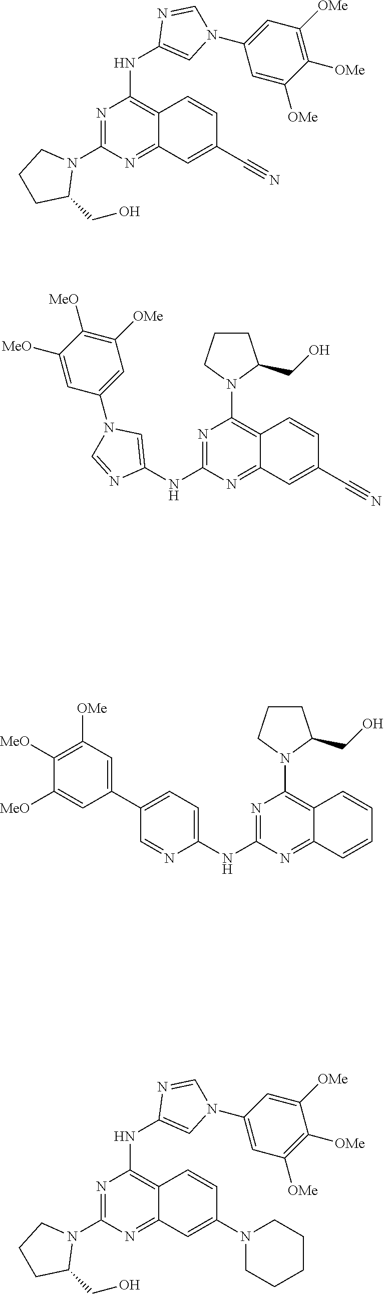 [SMILES: COc1cc(-n2cnc(Nc3nc(N4CCC[C@H]4CO)nc4cc(C#N)ccc34)c2)cc(OC)c1OC ; COc1cc(-n2cnc(Nc3nc(N4CCC[C@H]4CO)c4ccc(C#N)cc4n3)c2)cc(OC)c1OC ; COc1cc(-c2ccc(Nc3nc(N4CCC[C@H]4CO)c4ccccc4n3)nc2)cc(OC)c1OC ; COc1cc(-n2cnc(Nc3nc(N4CCC[C@H]4CO)nc4cc(N5CCCCC5)ccc34)c2)cc(OC)c1OC] 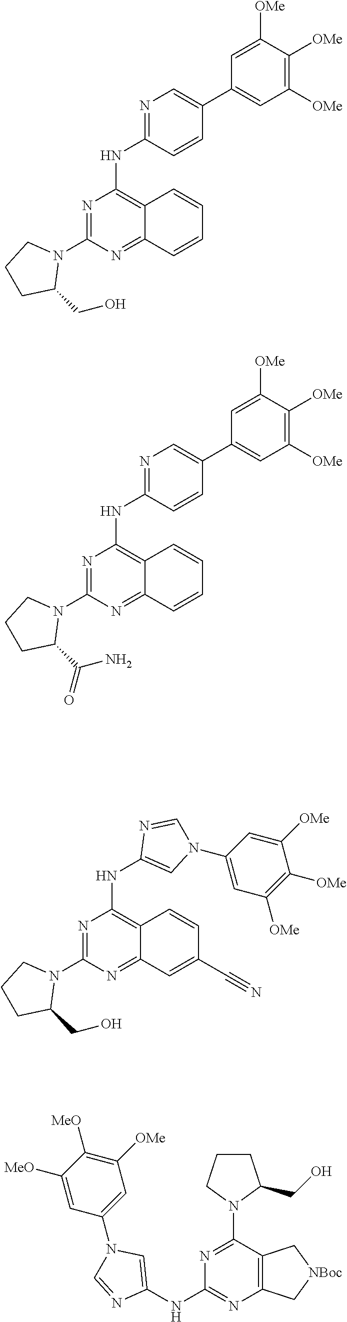 [SMILES: COc1cc(-c2ccc(Nc3nc(N4CCC[C@H]4CO)nc4ccccc34)nc2)cc(OC)c1OC ; COc1cc(-c2ccc(Nc3nc(N4CCC[C@H]4C(N)=O)nc4ccccc34)nc2)cc(OC)c1OC ; COc1cc(-n2cnc(Nc3nc(N4CCC[C@@H]4CO)nc4cc(C#N)ccc34)c2)cc(OC)c1OC ; COc1cc(-n2cnc(Nc3nc4c(c(N5CCC[C@H]5CO)n3)CN(C(=O)OC(C)(C)C)C4)c2)cc(OC)c1OC] 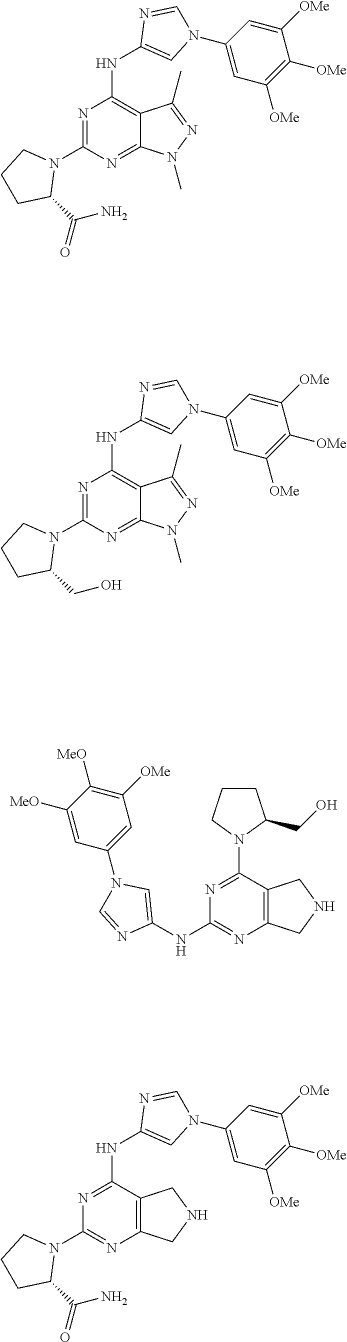 [SMILES: COc1cc(-n2cnc(Nc3nc(N4CCC[C@H]4C(N)=O)nc4c3c(C)nn4C)c2)cc(OC)c1OC ; COc1cc(-n2cnc(Nc3nc(N4CCC[C@H]4CO)nc4c3c(C)nn4C)c2)cc(OC)c1OC ; COc1cc(-n2cnc(Nc3nc4c(c(N5CCC[C@H]5CO)n3)CNC4)c2)cc(OC)c1OC ; COc1cc(-n2cnc(Nc3nc(N4CCC[C@H]4C(N)=O)nc4c3CNC4)c2)cc(OC)c1OC] 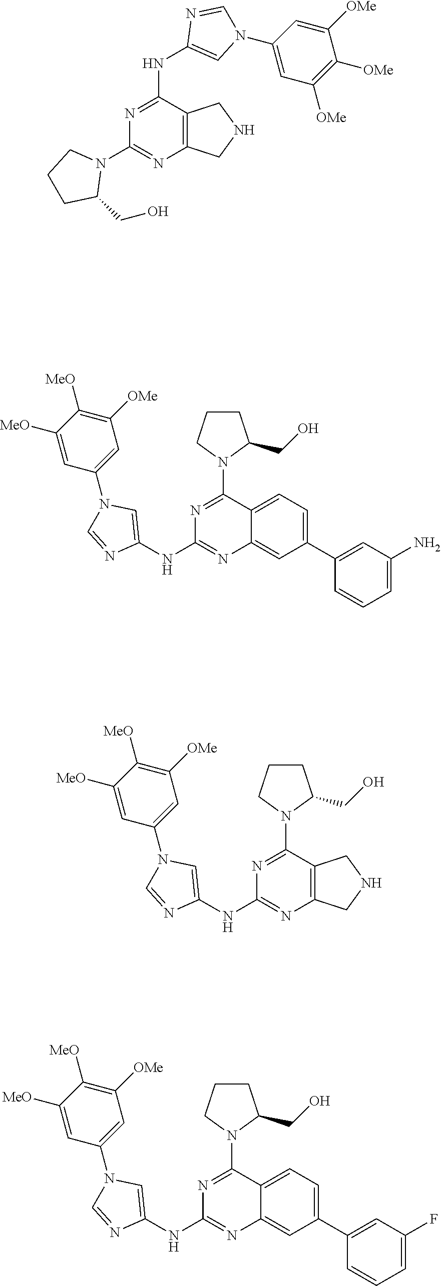 [SMILES: COc1cc(-n2cnc(Nc3nc(N4CCC[C@H]4CO)nc4c3CNC4)c2)cc(OC)c1OC ; COc1cc(-n2cnc(Nc3nc(N4CCC[C@H]4CO)c4ccc(-c5cccc(N)c5)cc4n3)c2)cc(OC)c1OC ; COc1cc(-n2cnc(Nc3nc4c(c(N5CCC[C@@H]5CO)n3)CNC4)c2)cc(OC)c1OC ; COc1cc(-n2cnc(Nc3nc(N4CCC[C@H]4CO)c4ccc(-c5cccc(F)c5)cc4n3)c2)cc(OC)c1OC] 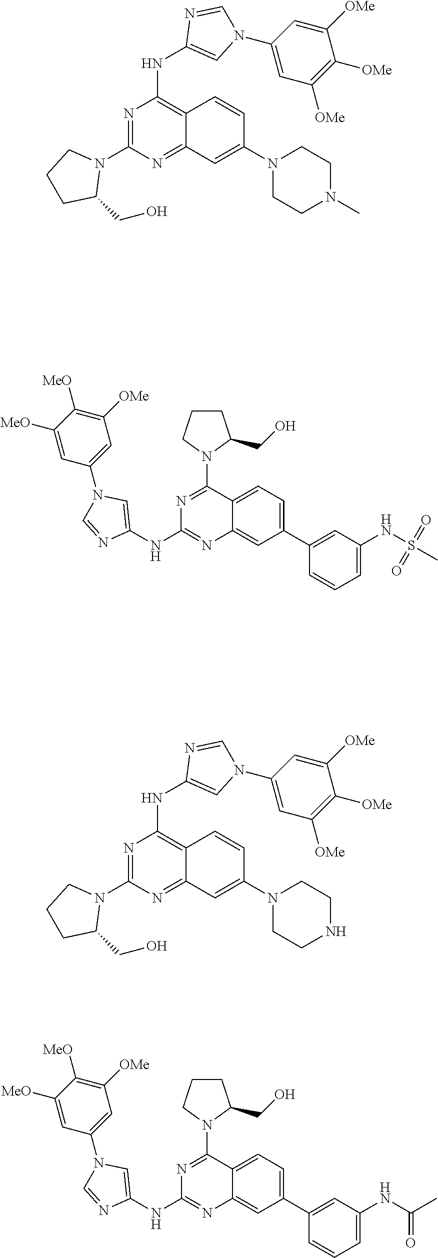 [SMILES: COc1cc(-n2cnc(Nc3nc(N4CCC[C@H]4CO)nc4cc(N5CCN(C)CC5)ccc34)c2)cc(OC)c1OC ; COc1cc(-n2cnc(Nc3nc(N4CCC[C@H]4CO)c4ccc(-c5cccc(NS(C)(=O)=O)c5)cc4n3)c2)cc(OC)c1OC ; COc1cc(-n2cnc(Nc3nc(N4CCC[C@H]4CO)nc4cc(N5CCNCC5)ccc34)c2)cc(OC)c1OC ; COc1cc(-n2cnc(Nc3nc(N4CCC[C@H]4CO)c4ccc(-c5cccc(NC(C)=O)c5)cc4n3)c2)cc(OC)c1OC] 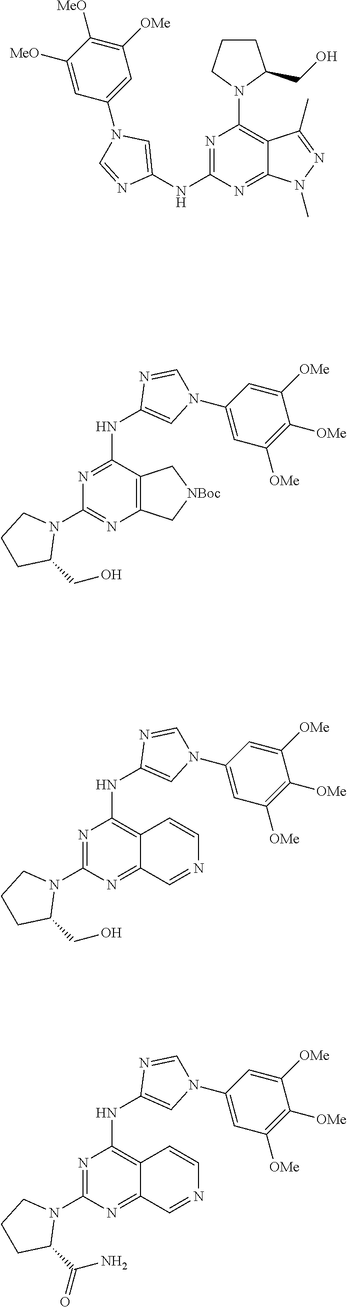 [SMILES: COc1cc(-n2cnc(Nc3nc(N4CCC[C@H]4CO)c4c(C)nn(C)c4n3)c2)cc(OC)c1OC ; COc1cc(-n2cnc(Nc3nc(N4CCC[C@H]4CO)nc4c3CN(C(=O)OC(C)(C)C)C4)c2)cc(OC)c1OC ; COc1cc(-n2cnc(Nc3nc(N4CCC[C@H]4CO)nc4cnccc34)c2)cc(OC)c1OC ; COc1cc(-n2cnc(Nc3nc(N4CCC[C@H]4C(N)=O)nc4cnccc34)c2)cc(OC)c1OC] 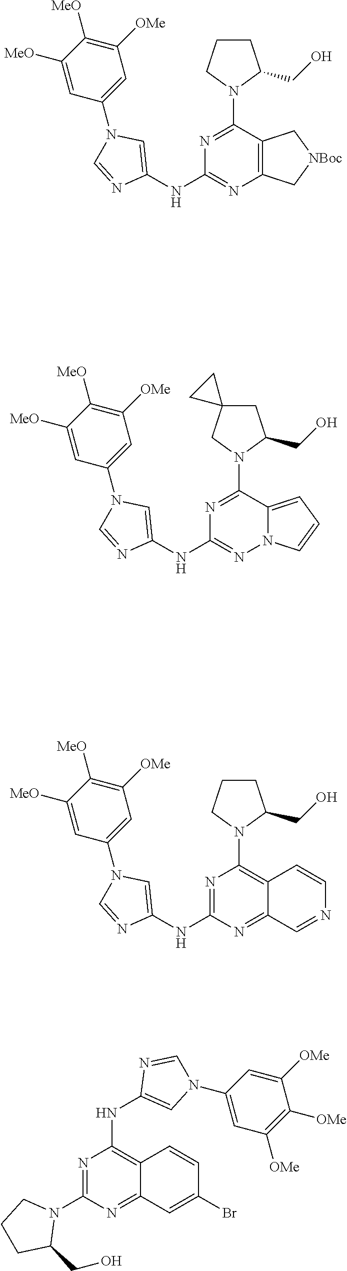 [SMILES: COc1cc(-n2cnc(Nc3nc4c(c(N5CCC[C@@H]5CO)n3)CN(C(=O)OC(C)(C)C)C4)c2)cc(OC)c1OC ; COc1cc(-n2cnc(Nc3nc(N4CC5(CC5)C[C@H]4CO)c4cccn4n3)c2)cc(OC)c1OC ; COc1cc(-n2cnc(Nc3nc(N4CCC[C@H]4CO)c4ccncc4n3)c2)cc(OC)c1OC ; COc1cc(-n2cnc(Nc3nc(N4CCC[C@@H]4CO)nc4cc(Br)ccc34)c2)cc(OC)c1OC] 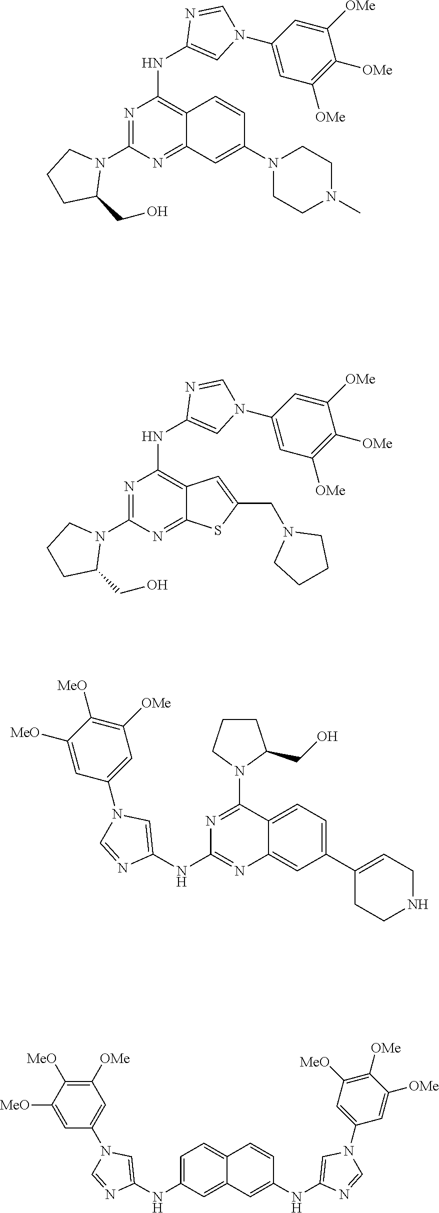 [SMILES: COc1cc(-n2cnc(Nc3nc(N4CCC[C@@H]4CO)nc4cc(N5CCN(C)CC5)ccc34)c2)cc(OC)c1OC ; COc1cc(-n2cnc(Nc3nc(N4CCC[C@H]4CO)nc4sc(CN5CCCC5)cc34)c2)cc(OC)c1OC ; COc1cc(-n2cnc(Nc3nc(N4CCC[C@H]4CO)c4ccc(C5=CCNCC5)cc4n3)c2)cc(OC)c1OC ; COc1cc(-n2cnc(Nc3ccc4ccc(Nc5cn(-c6cc(OC)c(OC)c(OC)c6)cn5)cc4c3)c2)cc(OC)c1OC] 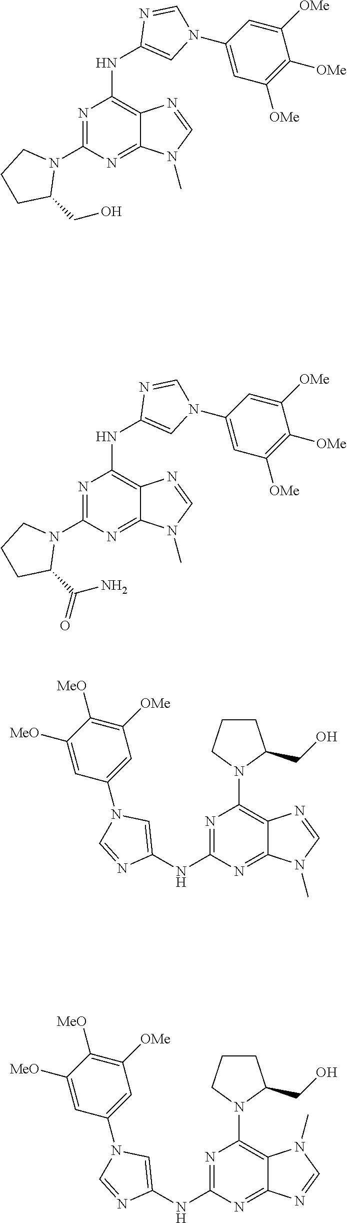 [SMILES: COc1cc(-n2cnc(Nc3nc(N4CCC[C@H]4CO)nc4c3ncn4C)c2)cc(OC)c1OC ; COc1cc(-n2cnc(Nc3nc(N4CCC[C@H]4C(N)=O)nc4c3ncn4C)c2)cc(OC)c1OC ; COc1cc(-n2cnc(Nc3nc(N4CCC[C@H]4CO)c4ncn(C)c4n3)c2)cc(OC)c1OC ; COc1cc(-n2cnc(Nc3nc(N4CCC[C@H]4CO)c4c(ncn4C)n3)c2)cc(OC)c1OC] 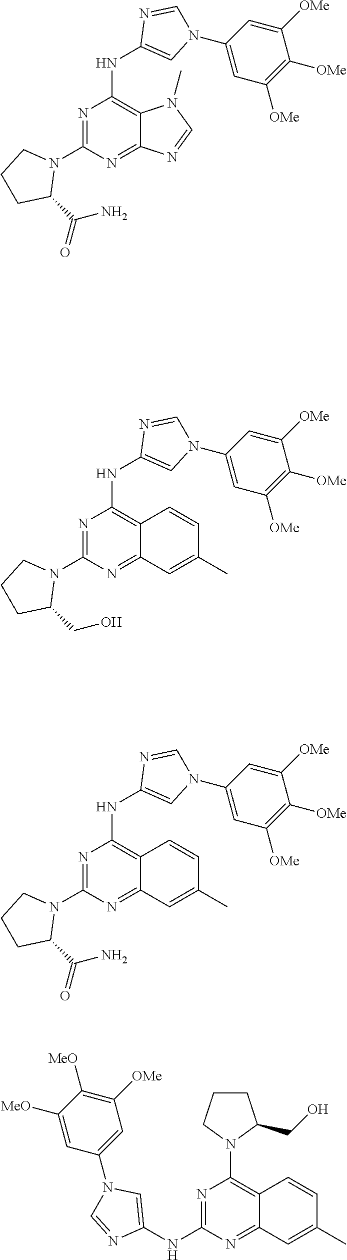 [SMILES: COc1cc(-n2cnc(Nc3nc(N4CCC[C@H]4C(N)=O)nc4ncn(C)c34)c2)cc(OC)c1OC ; COc1cc(-n2cnc(Nc3nc(N4CCC[C@H]4CO)nc4cc(C)ccc34)c2)cc(OC)c1OC ; COc1cc(-n2cnc(Nc3nc(N4CCC[C@H]4C(N)=O)nc4cc(C)ccc34)c2)cc(OC)c1OC ; COc1cc(-n2cnc(Nc3nc(N4CCC[C@H]4CO)c4ccc(C)cc4n3)c2)cc(OC)c1OC] 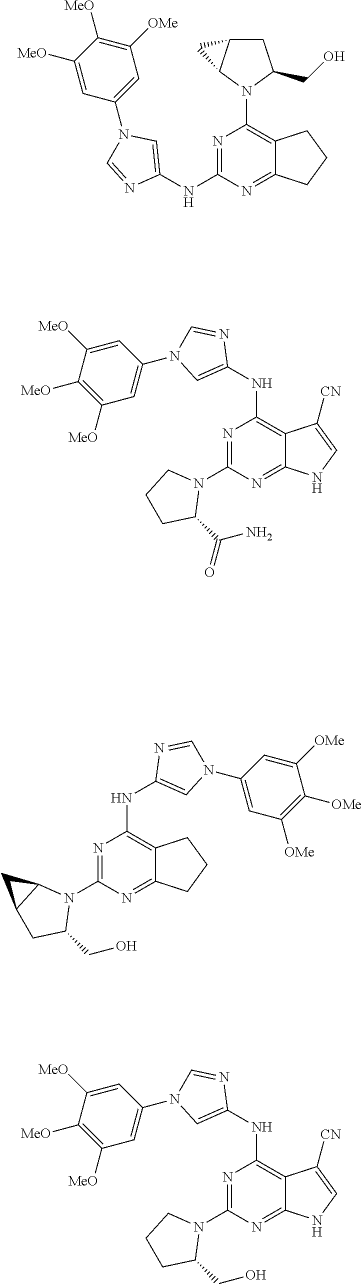 [SMILES: COc1cc(-n2cnc(Nc3nc4c(c(N5[C@H](CO)C[C@H]6C[C@H]65)n3)CCC4)c2)cc(OC)c1OC ; COc1cc(-n2cnc(Nc3nc(N4CCC[C@H]4C(N)=O)nc4[nH]cc(C#N)c34)c2)cc(OC)c1OC ; COc1cc(-n2cnc(Nc3nc(N4[C@H](CO)C[C@H]5C[C@H]54)nc4c3CCC4)c2)cc(OC)c1OC ; COc1cc(-n2cnc(Nc3nc(N4CCC[C@H]4CO)nc4[nH]cc(C#N)c34)c2)cc(OC)c1OC] 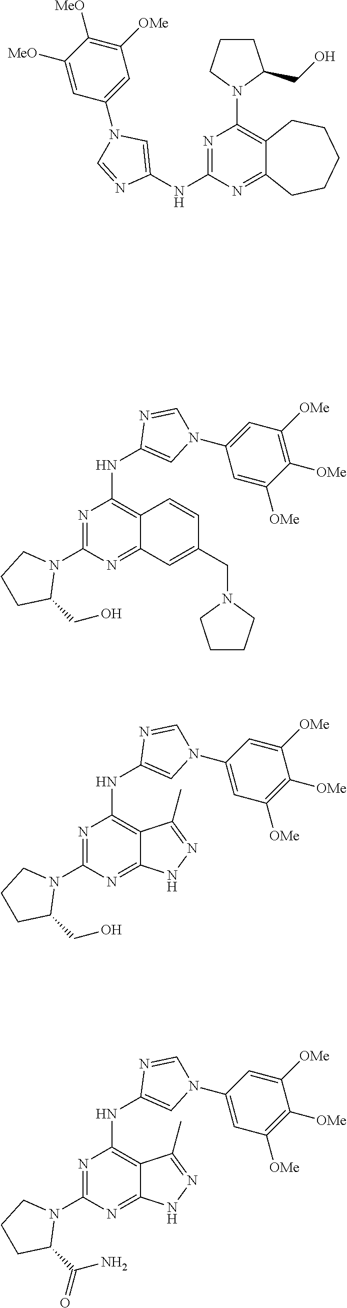 [SMILES: COc1cc(-n2cnc(Nc3nc4c(c(N5CCC[C@H]5CO)n3)CCCCC4)c2)cc(OC)c1OC ; COc1cc(-n2cnc(Nc3nc(N4CCC[C@H]4CO)nc4cc(CN5CCCC5)ccc34)c2)cc(OC)c1OC ; COc1cc(-n2cnc(Nc3nc(N4CCC[C@H]4CO)nc4[nH]nc(C)c34)c2)cc(OC)c1OC ; COc1cc(-n2cnc(Nc3nc(N4CCC[C@H]4C(N)=O)nc4[nH]nc(C)c34)c2)cc(OC)c1OC] 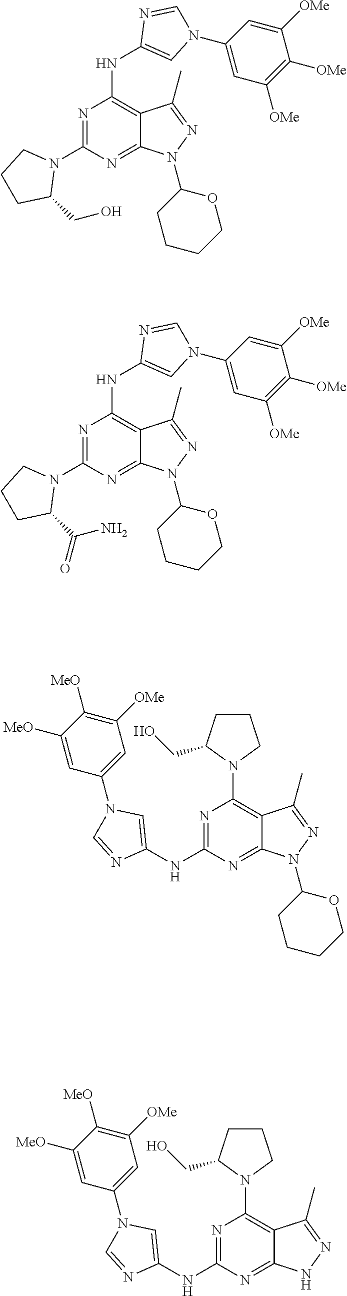 [SMILES: COc1cc(-n2cnc(Nc3nc(N4CCC[C@H]4CO)nc4c3c(C)nn4C3CCCCO3)c2)cc(OC)c1OC ; COc1cc(-n2cnc(Nc3nc(N4CCC[C@H]4C(N)=O)nc4c3c(C)nn4C3CCCCO3)c2)cc(OC)c1OC ; COc1cc(-n2cnc(Nc3nc(N4CCC[C@H]4CO)c4c(C)nn(C5CCCCO5)c4n3)c2)cc(OC)c1OC ; COc1cc(-n2cnc(Nc3nc(N4CCC[C@H]4CO)c4c(C)n[nH]c4n3)c2)cc(OC)c1OC] 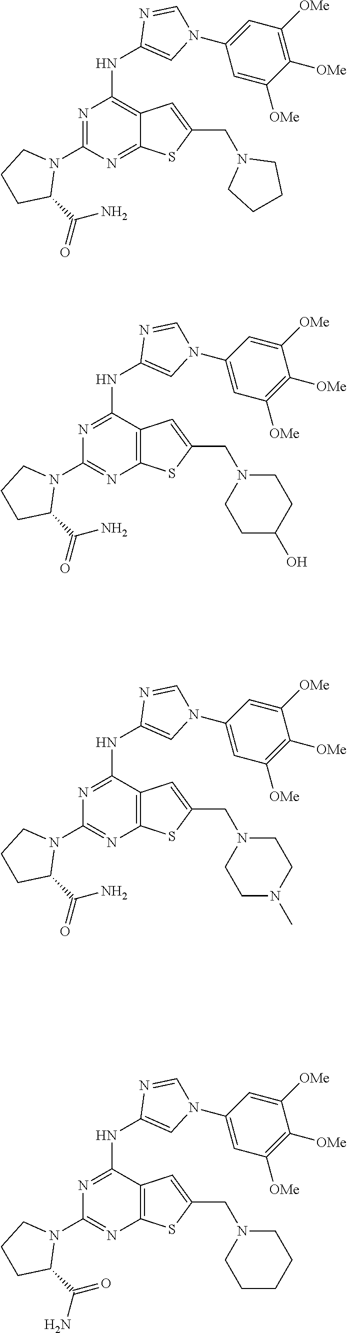 [SMILES: COc1cc(-n2cnc(Nc3nc(N4CCC[C@H]4C(N)=O)nc4sc(CN5CCCC5)cc34)c2)cc(OC)c1OC ; COc1cc(-n2cnc(Nc3nc(N4CCC[C@H]4C(N)=O)nc4sc(CN5CCC(O)CC5)cc34)c2)cc(OC)c1OC ; COc1cc(-n2cnc(Nc3nc(N4CCC[C@H]4C(N)=O)nc4sc(CN5CCN(C)CC5)cc34)c2)cc(OC)c1OC ; COc1cc(-n2cnc(Nc3nc(N4CCC[C@H]4C(N)=O)nc4sc(CN5CCCCC5)cc34)c2)cc(OC)c1OC] 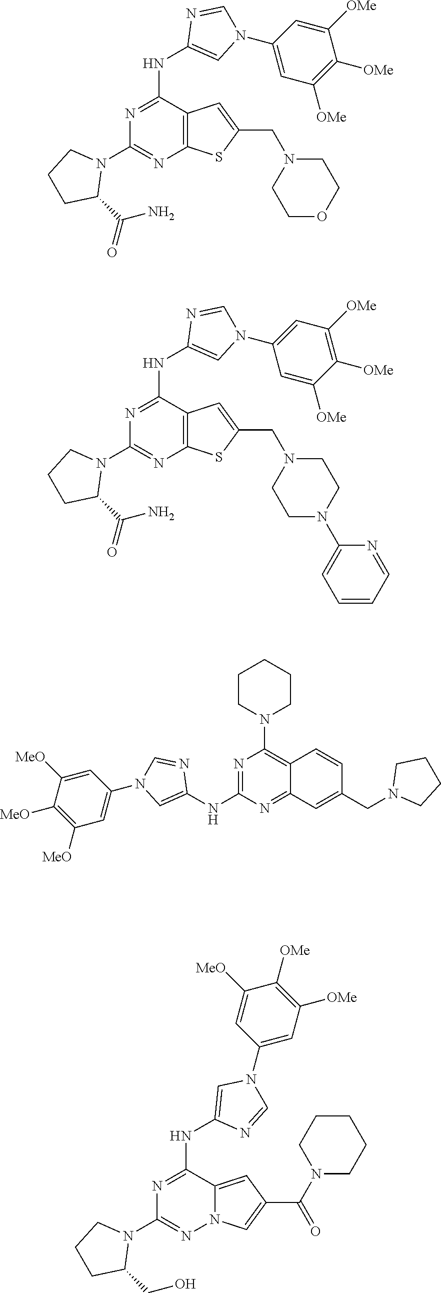 [SMILES: COc1cc(-n2cnc(Nc3nc(N4CCC[C@H]4C(N)=O)nc4sc(CN5CCOCC5)cc34)c2)cc(OC)c1OC ; COc1cc(-n2cnc(Nc3nc(N4CCC[C@H]4C(N)=O)nc4sc(CN5CCN(c6ccccn6)CC5)cc34)c2)cc(OC)c1OC ; COc1cc(-n2cnc(Nc3nc(N4CCCCC4)c4ccc(CN5CCCC5)cc4n3)c2)cc(OC)c1OC ; COc1cc(-n2cnc(Nc3nc(N4CCC[C@H]4CO)nn4cc(C(=O)N5CCCCC5)cc34)c2)cc(OC)c1OC] 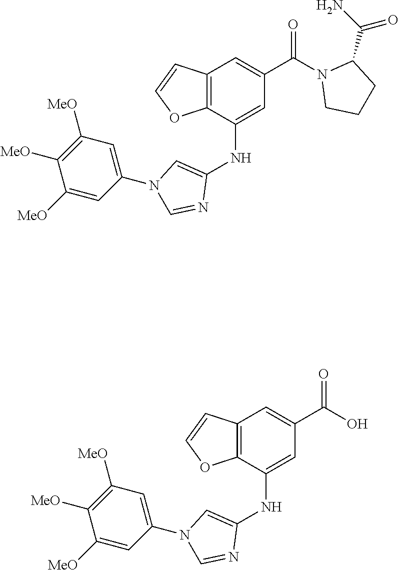 [SMILES: COc1cc(-n2cnc(Nc3cc(C(=O)N4CCC[C@H]4C(N)=O)cc4ccoc34)c2)cc(OC)c1OC ; COc1cc(-n2cnc(Nc3cc(C(=O)O)cc4ccoc34)c2)cc(OC)c1OC] 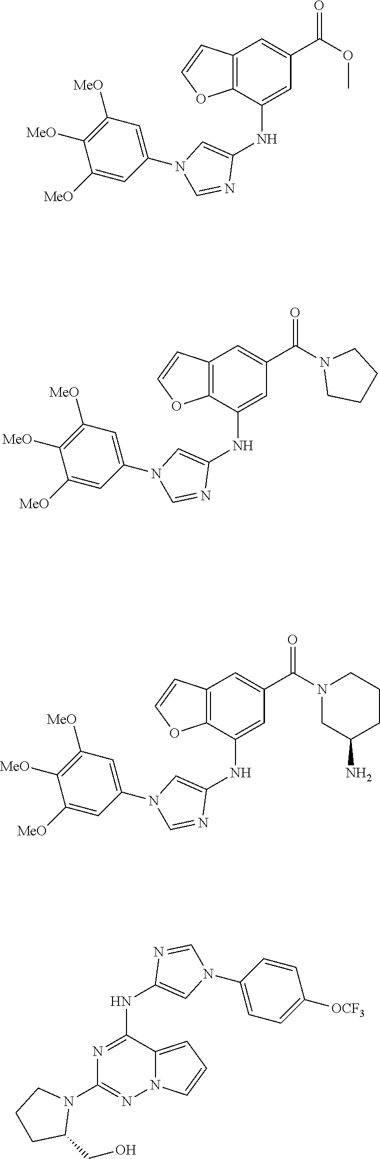 [SMILES: COC(=O)c1cc(Nc2cn(-c3cc(OC)c(OC)c(OC)c3)cn2)c2occc2c1 ; COc1cc(-n2cnc(Nc3cc(C(=O)N4CCCC4)cc4ccoc34)c2)cc(OC)c1OC ; COc1cc(-n2cnc(Nc3cc(C(=O)N4CCC[C@@H](N)C4)cc4ccoc34)c2)cc(OC)c1OC ; OC[C@@H]1CCCN1c1nc(Nc2cn(-c3ccc(OC(F)(F)F)cc3)cn2)c2cccn2n1] 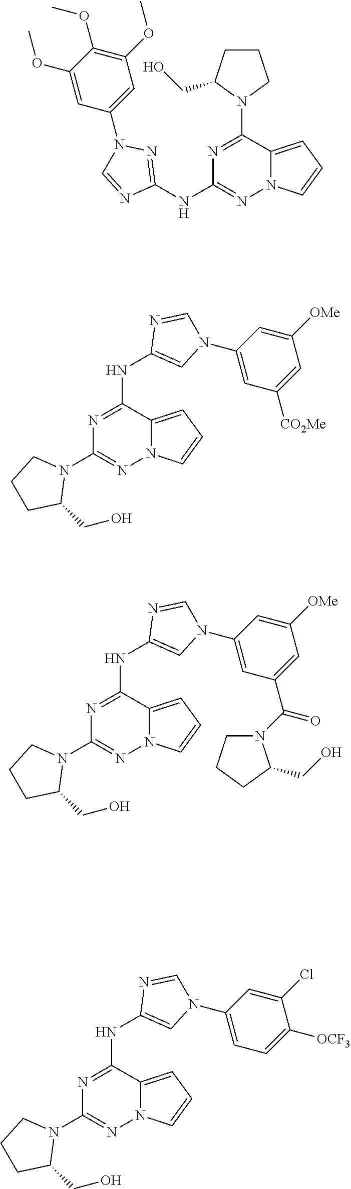 [SMILES: COc1cc(-n2cnc(Nc3nc(N4CCC[C@H]4CO)c4cccn4n3)n2)cc(OC)c1OC ; COC(=O)c1cc(OC)cc(-n2cnc(Nc3nc(N4CCC[C@H]4CO)nn4cccc34)c2)c1 ; COc1cc(C(=O)N2CCC[C@H]2CO)cc(-n2cnc(Nc3nc(N4CCC[C@H]4CO)nn4cccc34)c2)c1 ; OC[C@@H]1CCCN1c1nc(Nc2cn(-c3ccc(OC(F)(F)F)c(Cl)c3)cn2)c2cccn2n1] 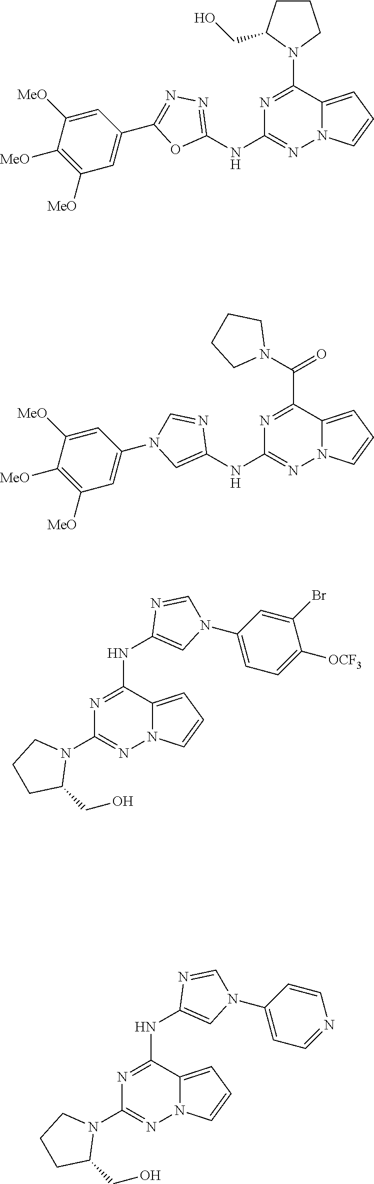 [SMILES: COc1cc(-c2nnc(Nc3nc(N4CCC[C@H]4CO)c4cccn4n3)o2)cc(OC)c1OC ; COc1cc(-n2cnc(Nc3nc(C(=O)N4CCCC4)c4cccn4n3)c2)cc(OC)c1OC ; OC[C@@H]1CCCN1c1nc(Nc2cn(-c3ccc(OC(F)(F)F)c(Br)c3)cn2)c2cccn2n1 ; OC[C@@H]1CCCN1c1nc(Nc2cn(-c3ccncc3)cn2)c2cccn2n1] 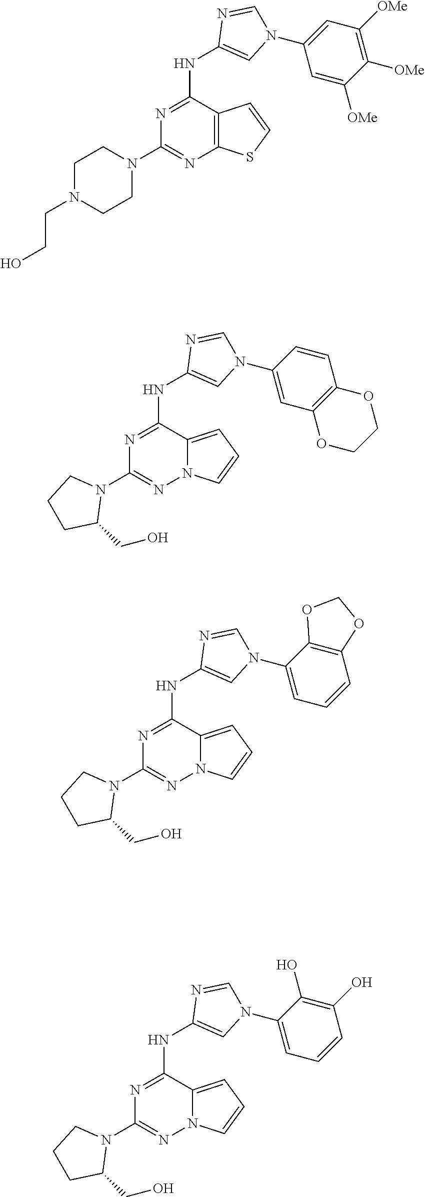 [SMILES: COc1cc(-n2cnc(Nc3nc(N4CCN(CCO)CC4)nc4sccc34)c2)cc(OC)c1OC ; OC[C@@H]1CCCN1c1nc(Nc2cn(-c3ccc4c(c3)OCCO4)cn2)c2cccn2n1 ; OC[C@@H]1CCCN1c1nc(Nc2cn(-c3cccc4c3OCO4)cn2)c2cccn2n1 ; OC[C@@H]1CCCN1c1nc(Nc2cn(-c3cccc(O)c3O)cn2)c2cccn2n1] 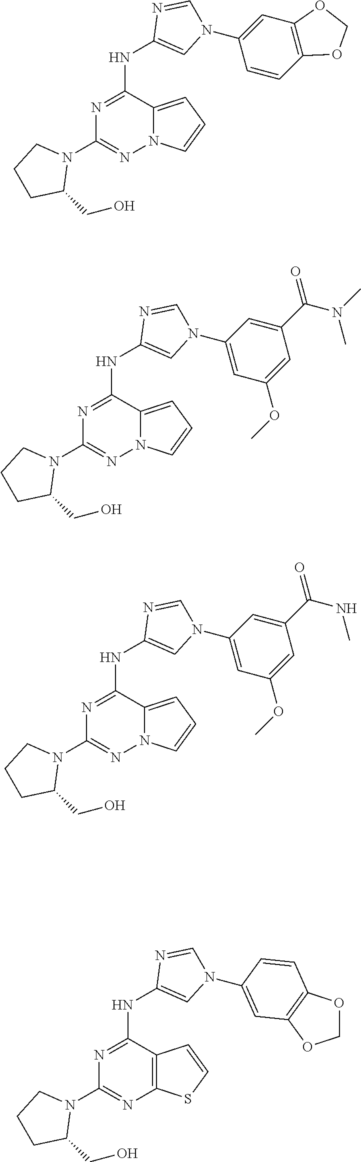 [SMILES: OC[C@@H]1CCCN1c1nc(Nc2cn(-c3ccc4c(c3)OCO4)cn2)c2cccn2n1 ; COc1cc(C(=O)N(C)C)cc(-n2cnc(Nc3nc(N4CCC[C@H]4CO)nn4cccc34)c2)c1 ; CNC(=O)c1cc(OC)cc(-n2cnc(Nc3nc(N4CCC[C@H]4CO)nn4cccc34)c2)c1 ; OC[C@@H]1CCCN1c1nc(Nc2cn(-c3ccc4c(c3)OCO4)cn2)c2ccsc2n1] 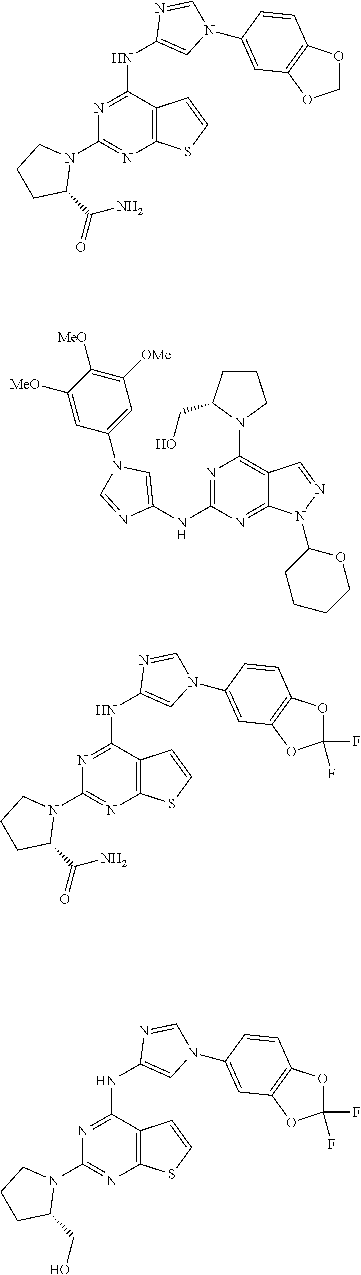 [SMILES: NC(=O)[C@@H]1CCCN1c1nc(Nc2cn(-c3ccc4c(c3)OCO4)cn2)c2ccsc2n1 ; COc1cc(-n2cnc(Nc3nc(N4CCC[C@H]4CO)c4cnn(C5CCCCO5)c4n3)c2)cc(OC)c1OC ; NC(=O)[C@@H]1CCCN1c1nc(Nc2cn(-c3ccc4c(c3)OC(F)(F)O4)cn2)c2ccsc2n1 ; OC[C@@H]1CCCN1c1nc(Nc2cn(-c3ccc4c(c3)OC(F)(F)O4)cn2)c2ccsc2n1] 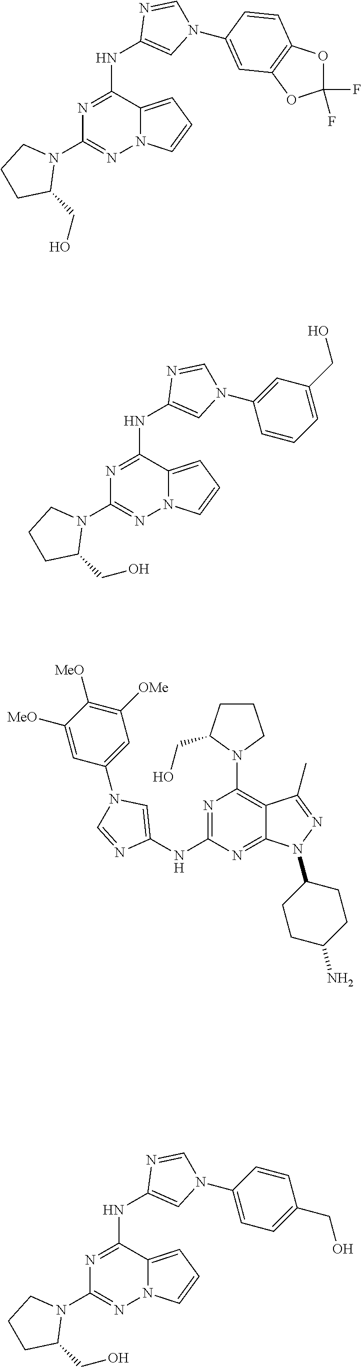 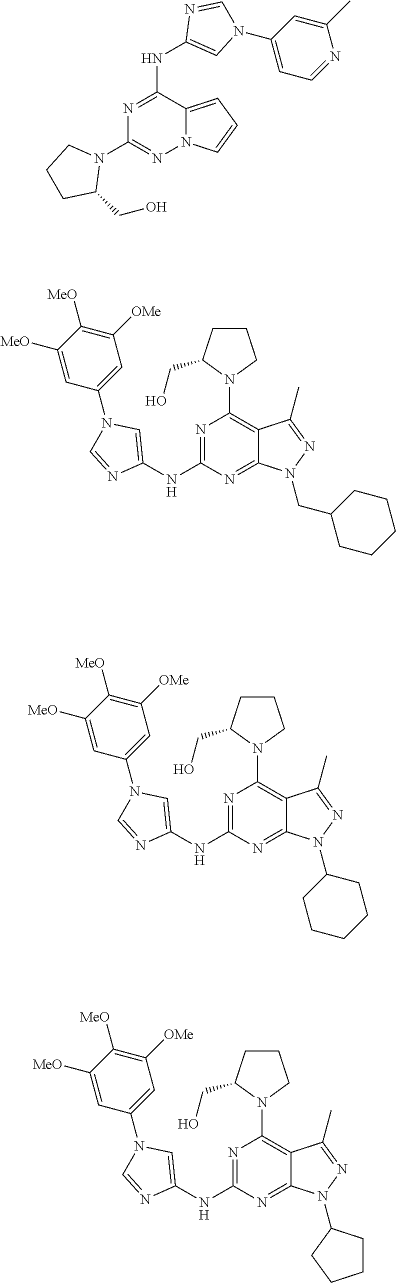 [SMILES: Cc1cc(-n2cnc(Nc3nc(N4CCC[C@H]4CO)nn4cccc34)c2)ccn1 ; COc1cc(-n2cnc(Nc3nc(N4CCC[C@H]4CO)c4c(C)nn(CC5CCCCC5)c4n3)c2)cc(OC)c1OC ; COc1cc(-n2cnc(Nc3nc(N4CCC[C@H]4CO)c4c(C)nn(C5CCCCC5)c4n3)c2)cc(OC)c1OC ; COc1cc(-n2cnc(Nc3nc(N4CCC[C@H]4CO)c4c(C)nn(C5CCCC5)c4n3)c2)cc(OC)c1OC] 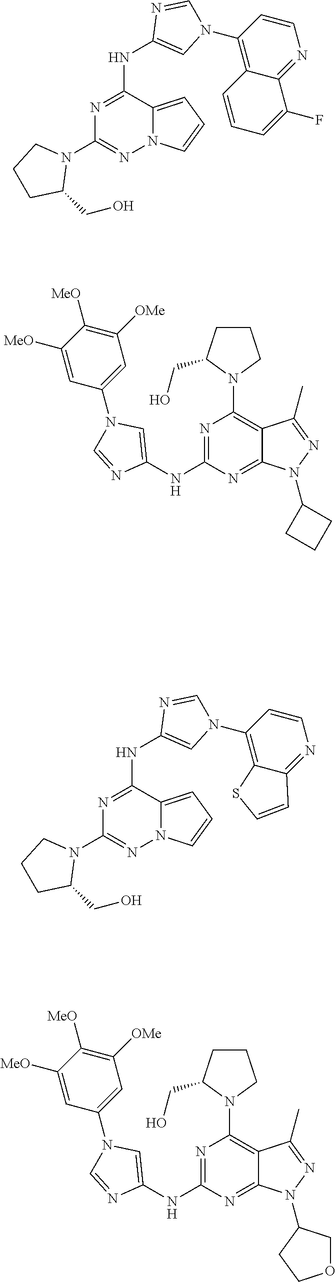 [SMILES: OC[C@@H]1CCCN1c1nc(Nc2cn(-c3ccnc4c(F)cccc34)cn2)c2cccn2n1 ; COc1cc(-n2cnc(Nc3nc(N4CCC[C@H]4CO)c4c(C)nn(C5CCC5)c4n3)c2)cc(OC)c1OC ; OC[C@@H]1CCCN1c1nc(Nc2cn(-c3ccnc4ccsc34)cn2)c2cccn2n1 ; COc1cc(-n2cnc(Nc3nc(N4CCC[C@H]4CO)c4c(C)nn(C5CCOC5)c4n3)c2)cc(OC)c1OC] 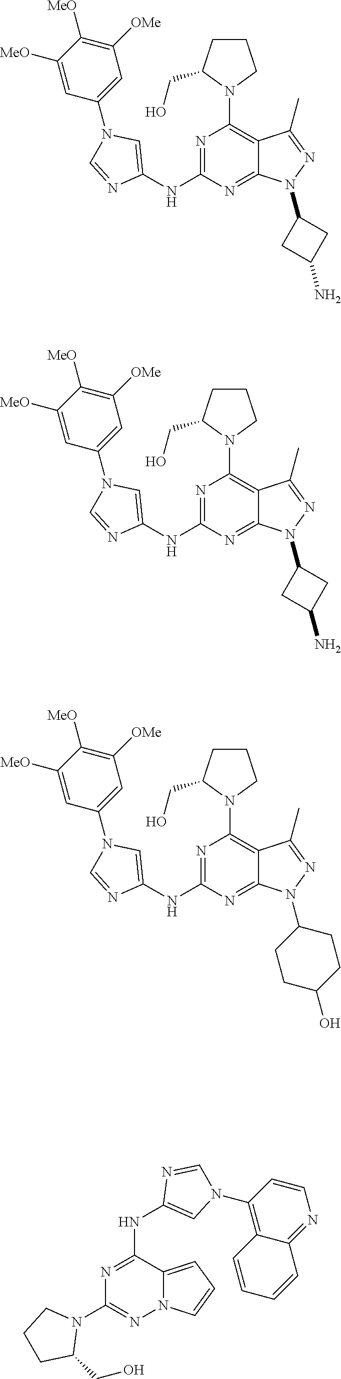 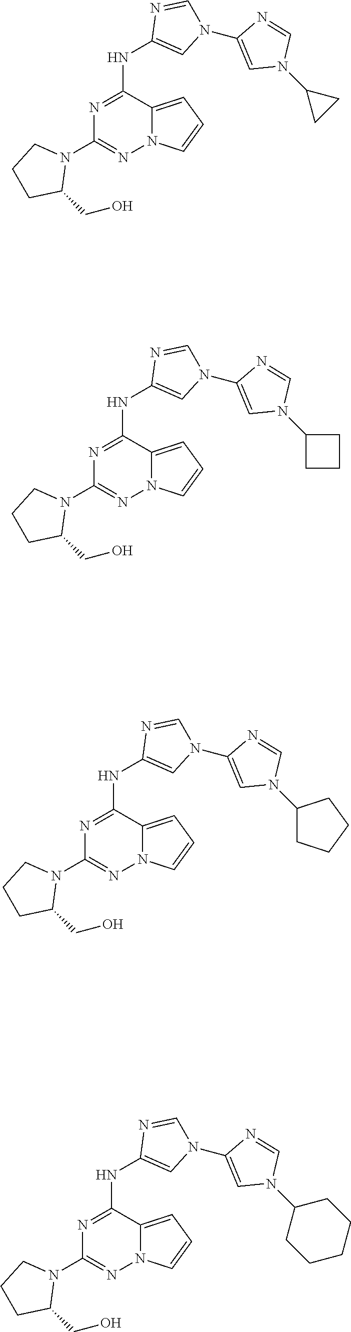 [SMILES: OC[C@@H]1CCCN1c1nc(Nc2cn(-c3cn(C4CC4)cn3)cn2)c2cccn2n1 ; OC[C@@H]1CCCN1c1nc(Nc2cn(-c3cn(C4CCC4)cn3)cn2)c2cccn2n1 ; OC[C@@H]1CCCN1c1nc(Nc2cn(-c3cn(C4CCCC4)cn3)cn2)c2cccn2n1 ; OC[C@@H]1CCCN1c1nc(Nc2cn(-c3cn(C4CCCCC4)cn3)cn2)c2cccn2n1] 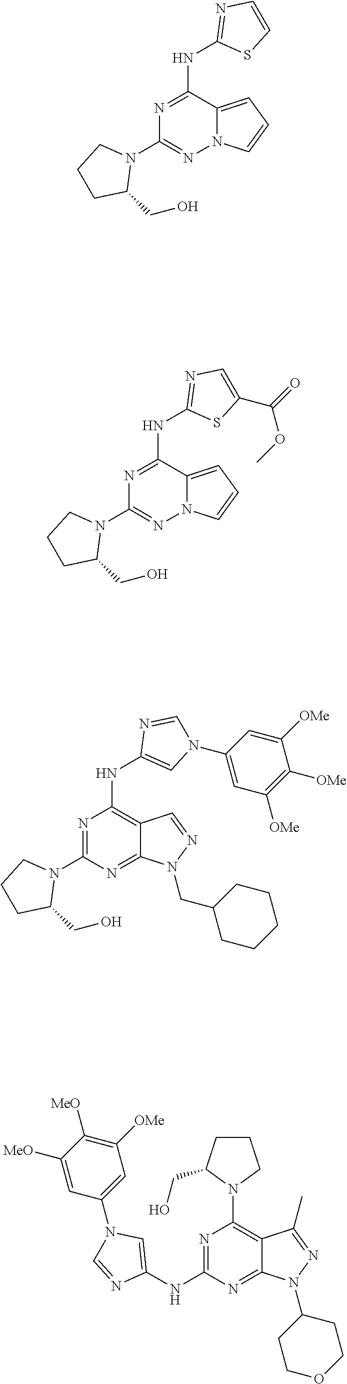 [SMILES: OC[C@@H]1CCCN1c1nc(Nc2nccs2)c2cccn2n1 ; COC(=O)c1cnc(Nc2nc(N3CCC[C@H]3CO)nn3cccc23)s1 ; COc1cc(-n2cnc(Nc3nc(N4CCC[C@H]4CO)nc4c3cnn4CC3CCCCC3)c2)cc(OC)c1OC ; COc1cc(-n2cnc(Nc3nc(N4CCC[C@H]4CO)c4c(C)nn(C5CCOCC5)c4n3)c2)cc(OC)c1OC] 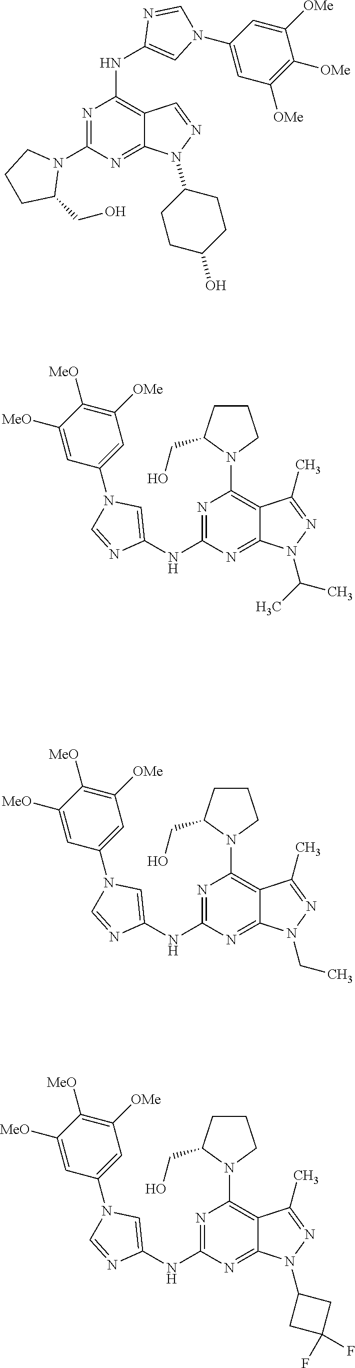 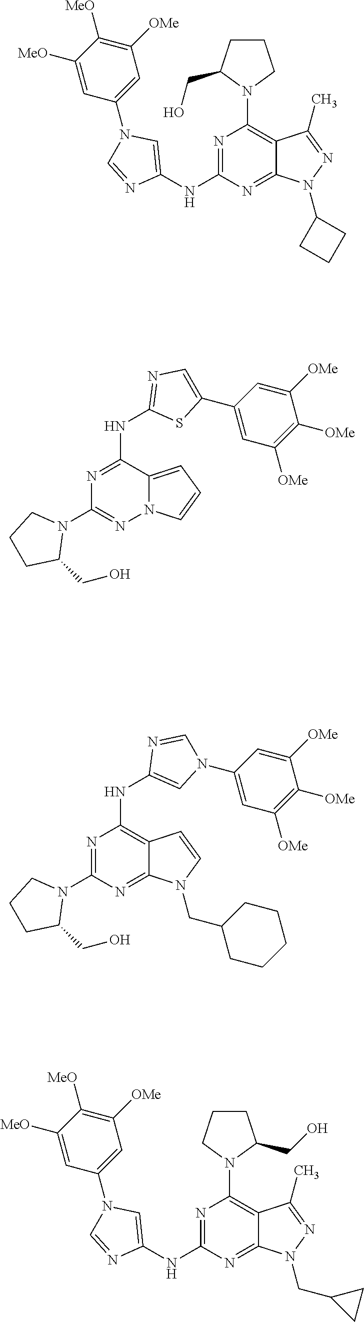 [SMILES: COc1cc(-n2cnc(Nc3nc(N4CCC[C@@H]4CO)c4c(C)nn(C5CCC5)c4n3)c2)cc(OC)c1OC ; COc1cc(-c2cnc(Nc3nc(N4CCC[C@H]4CO)nn4cccc34)s2)cc(OC)c1OC ; COc1cc(-n2cnc(Nc3nc(N4CCC[C@H]4CO)nc4c3ccn4CC3CCCCC3)c2)cc(OC)c1OC ; COc1cc(-n2cnc(Nc3nc(N4CCC[C@H]4CO)c4c(C)nn(CC5CC5)c4n3)c2)cc(OC)c1OC] 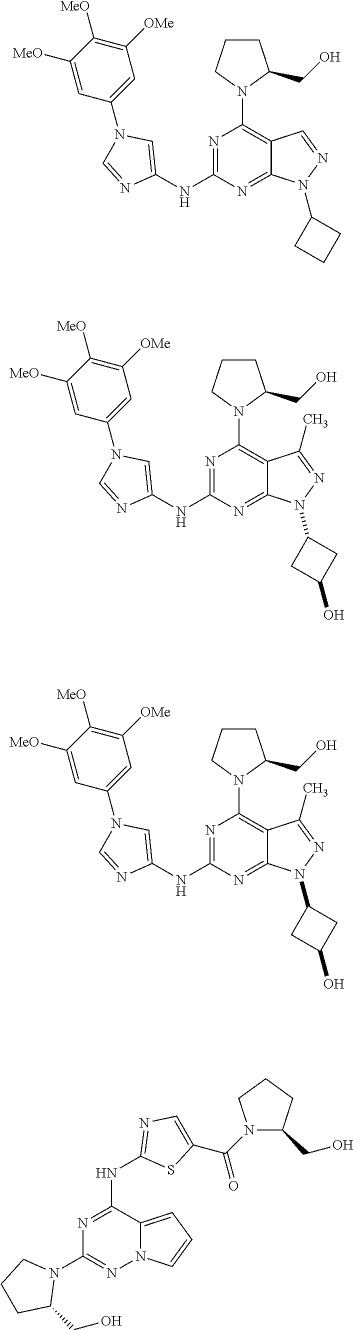 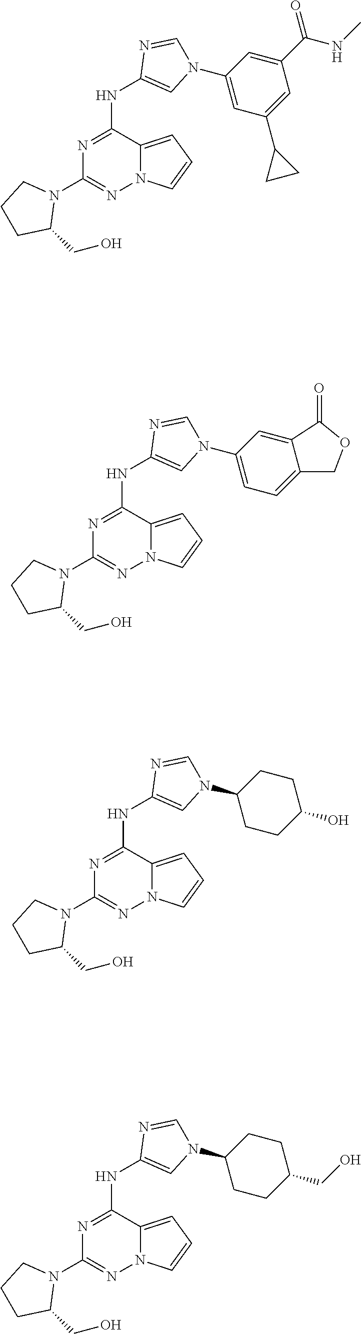 [SMILES: CNC(=O)c1cc(C2CC2)cc(-n2cnc(Nc3nc(N4CCC[C@H]4CO)nn4cccc34)c2)c1 ; O=C1OCc2ccc(-n3cnc(Nc4nc(N5CCC[C@H]5CO)nn5cccc45)c3)cc21 ; OC[C@@H]1CCCN1c1nc(Nc2cn([C@H]3CC[C@H](O)CC3)cn2)c2cccn2n1 ; OC[C@@H]1CCCN1c1nc(Nc2cn([C@H]3CC[C@H](CO)CC3)cn2)c2cccn2n1] 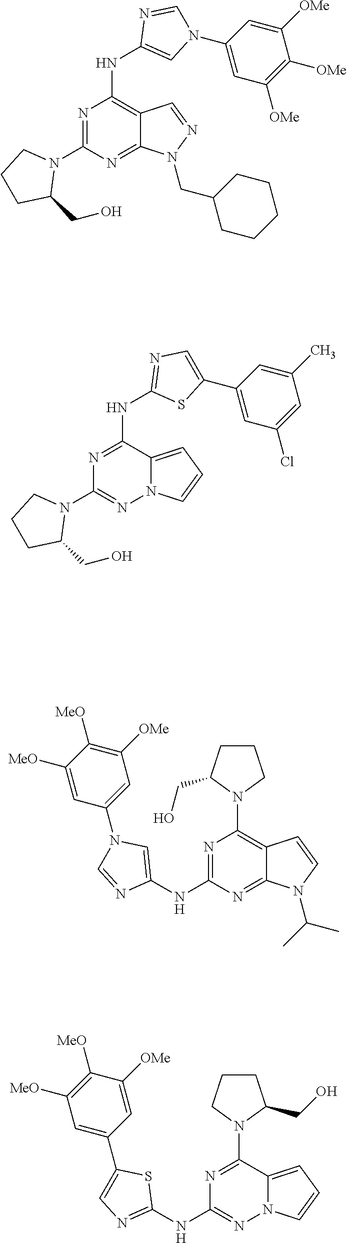 [SMILES: COc1cc(-n2cnc(Nc3nc(N4CCC[C@@H]4CO)nc4c3cnn4CC3CCCCC3)c2)cc(OC)c1OC ; Cc1cc(Cl)cc(-c2cnc(Nc3nc(N4CCC[C@H]4CO)nn4cccc34)s2)c1 ; COc1cc(-n2cnc(Nc3nc(N4CCC[C@H]4CO)c4ccn(C(C)C)c4n3)c2)cc(OC)c1OC ; COc1cc(-c2cnc(Nc3nc(N4CCC[C@H]4CO)c4cccn4n3)s2)cc(OC)c1OC] 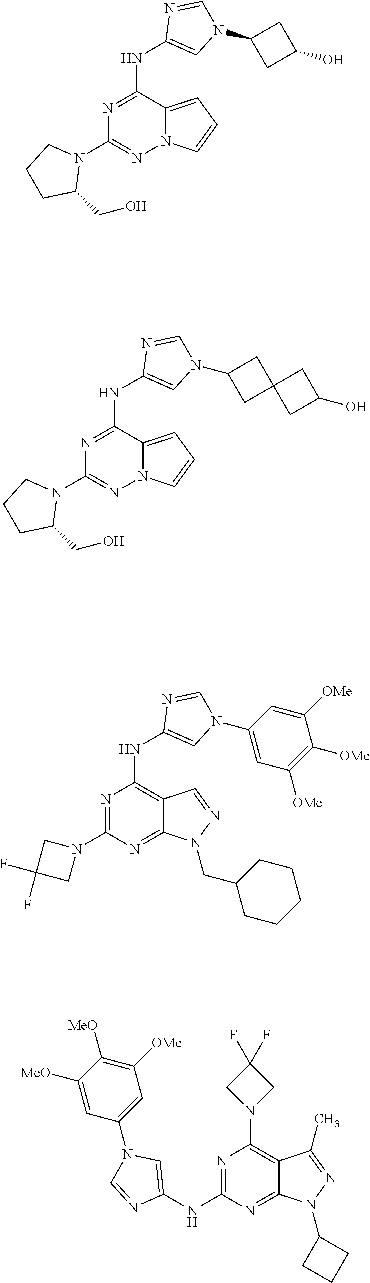 [SMILES: OC[C@@H]1CCCN1c1nc(Nc2cn([C@H]3C[C@H](O)C3)cn2)c2cccn2n1 ; OC[C@@H]1CCCN1c1nc(Nc2cn(C3CC4(CC(O)C4)C3)cn2)c2cccn2n1 ; COc1cc(-n2cnc(Nc3nc(N4CC(F)(F)C4)nc4c3cnn4CC3CCCCC3)c2)cc(OC)c1OC ; COc1cc(-n2cnc(Nc3nc(N4CC(F)(F)C4)c4c(C)nn(C5CCC5)c4n3)c2)cc(OC)c1OC] 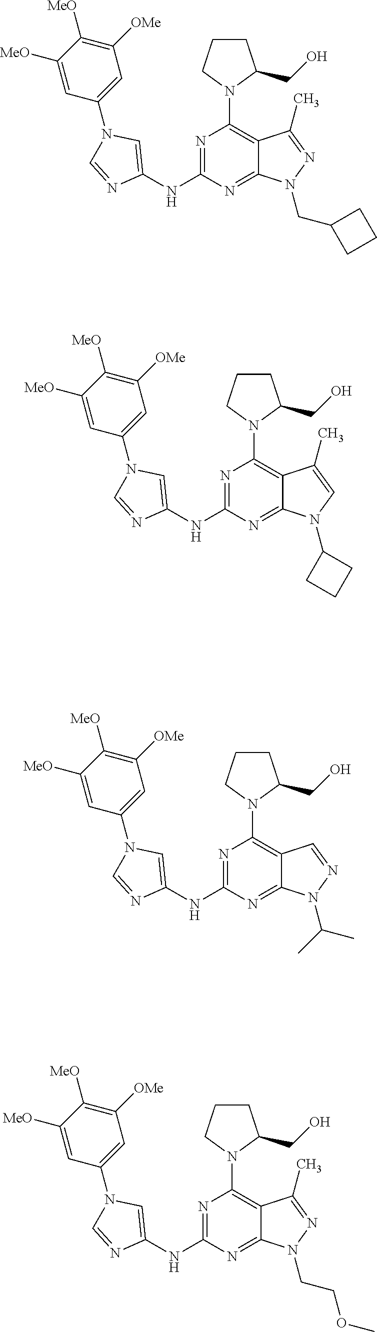 [SMILES: COc1cc(-n2cnc(Nc3nc(N4CCC[C@H]4CO)c4c(C)nn(CC5CCC5)c4n3)c2)cc(OC)c1OC ; COc1cc(-n2cnc(Nc3nc(N4CCC[C@H]4CO)c4c(C)cn(C5CCC5)c4n3)c2)cc(OC)c1OC ; COc1cc(-n2cnc(Nc3nc(N4CCC[C@H]4CO)c4cnn(C(C)C)c4n3)c2)cc(OC)c1OC ; COCCn1nc(C)c2c(N3CCC[C@H]3CO)nc(Nc3cn(-c4cc(OC)c(OC)c(OC)c4)cn3)nc21] 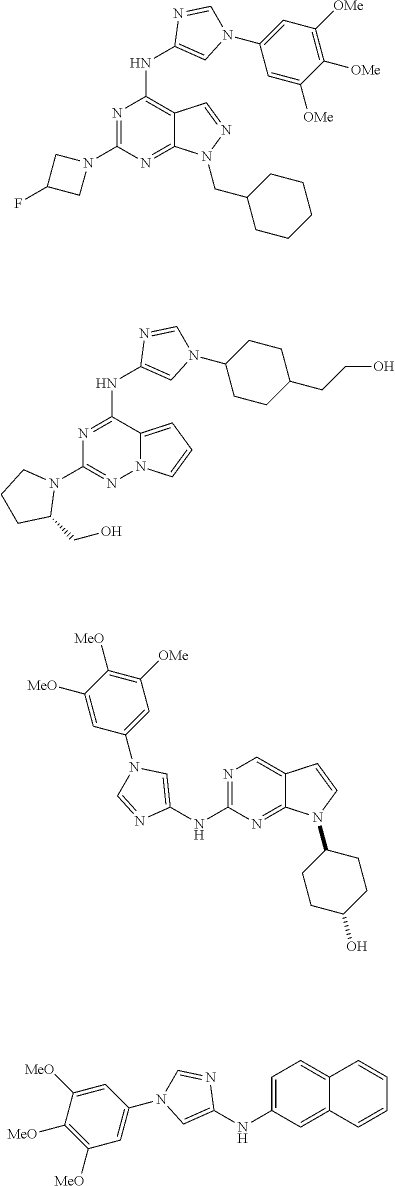 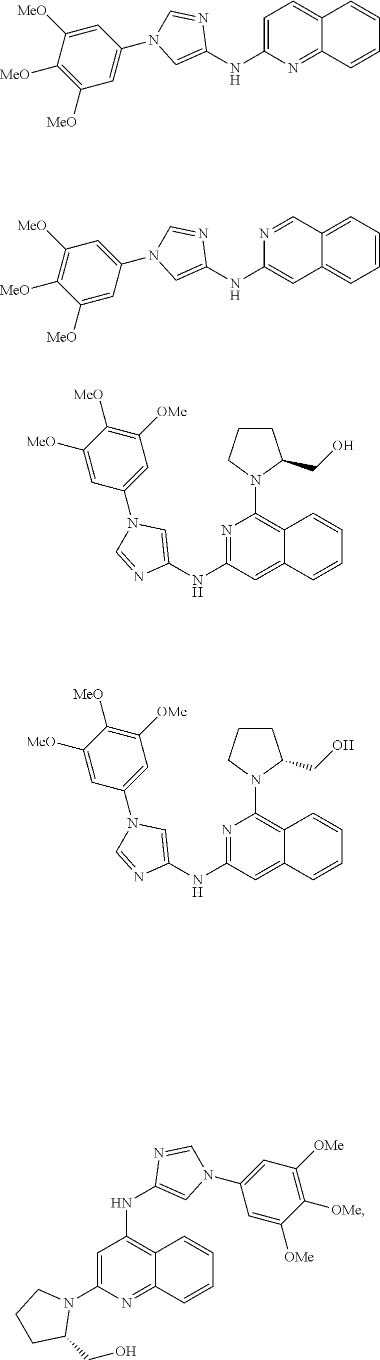 [SMILES: COc1cc(-n2cnc(Nc3ccc4ccccc4n3)c2)cc(OC)c1OC ; COc1cc(-n2cnc(Nc3cc4ccccc4cn3)c2)cc(OC)c1OC ; COc1cc(-n2cnc(Nc3cc4ccccc4c(N4CCC[C@H]4CO)n3)c2)cc(OC)c1OC ; COc1cc(-n2cnc(Nc3cc4ccccc4c(N4CCC[C@@H]4CO)n3)c2)cc(OC)c1OC ; COc1cc(-n2cnc(Nc3cc(N4CCC[C@H]4CO)nc4ccccc34)c2)cc(OC)c1OC]
or a pharmaceutically acceptable salt or prodrug thereof.

7. The method of claim 6, wherein the ALK2 kinase comprises a mutation.

8. The method of claim 7, wherein the mutation is an activating mutation.

9. The method of claim 8, wherein the activating mutation is R206H.

10. A method of treating fibrodysplasia ossificans progressiva, comprising orally administering to a subject in need thereof a therapeutically effective amount of a compound selected from: 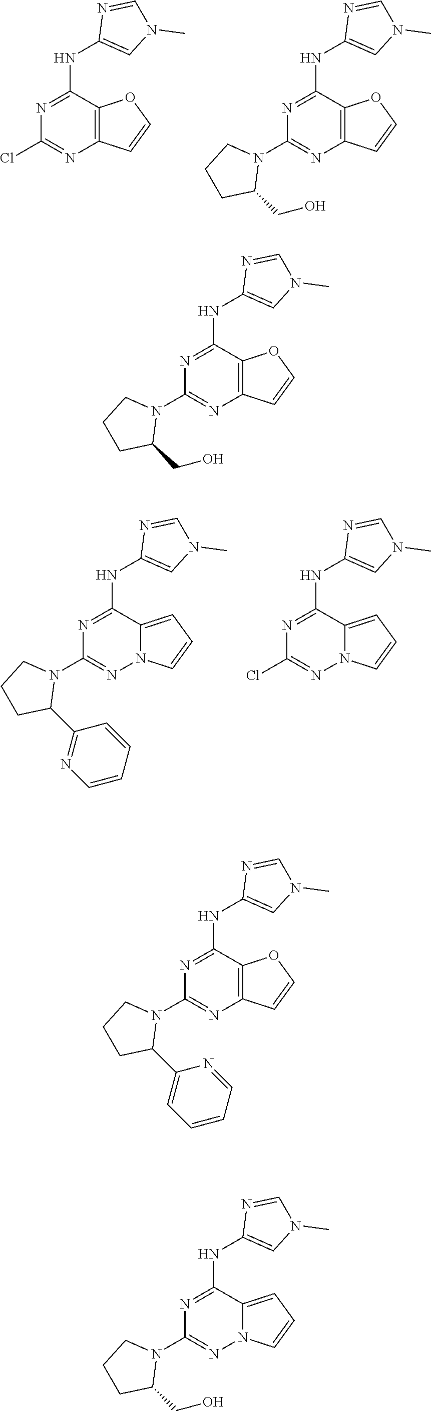 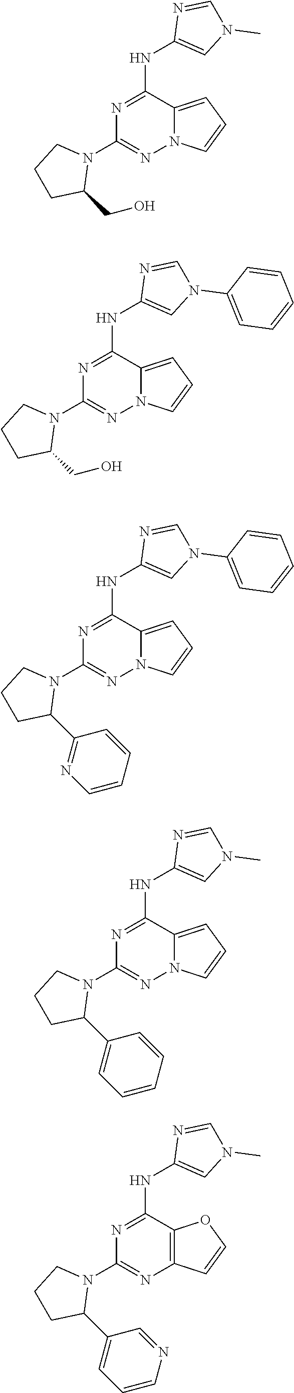 [SMILES: Cn1cnc(Nc2nc(N3CCC[C@@H]3CO)nn3cccc23)c1 ; OC[C@@H]1CCCN1c1nc(Nc2cn(-c3ccccc3)cn2)c2cccn2n1 ; c1ccc(-n2cnc(Nc3nc(N4CCCC4c4ccccn4)nn4cccc34)c2)cc1 ; Cn1cnc(Nc2nc(N3CCCC3c3ccccc3)nn3cccc23)c1 ; Cn1cnc(Nc2nc(N3CCCC3c3cccnc3)nc3ccoc23)c1] 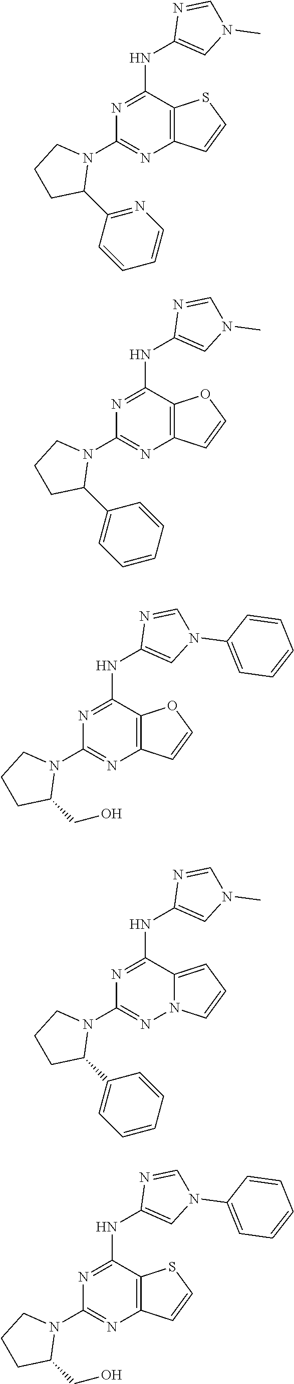 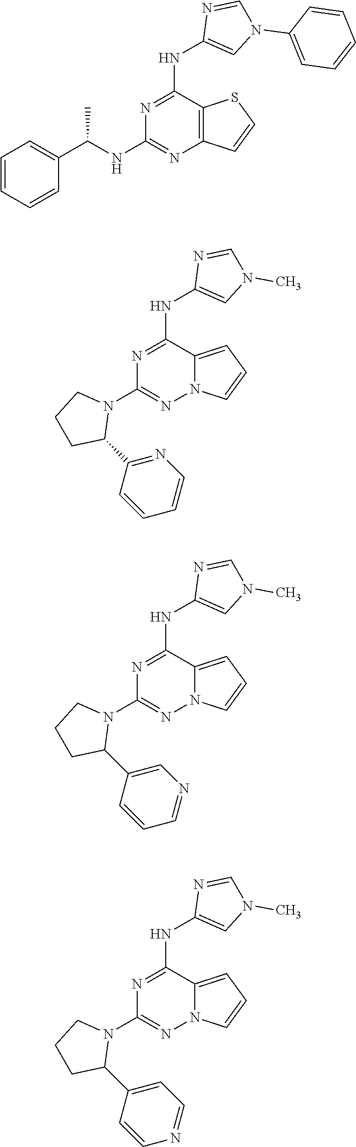 [SMILES: C[C@H](Nc1nc(Nc2cn(-c3ccccc3)cn2)c2sccc2n1)c1ccccc1 ; Cn1cnc(Nc2nc(N3CCC[C@H]3c3ccccn3)nn3cccc23)c1 ; Cn1cnc(Nc2nc(N3CCCC3c3cccnc3)nn3cccc23)c1 ; Cn1cnc(Nc2nc(N3CCCC3c3ccncc3)nn3cccc23)c1] 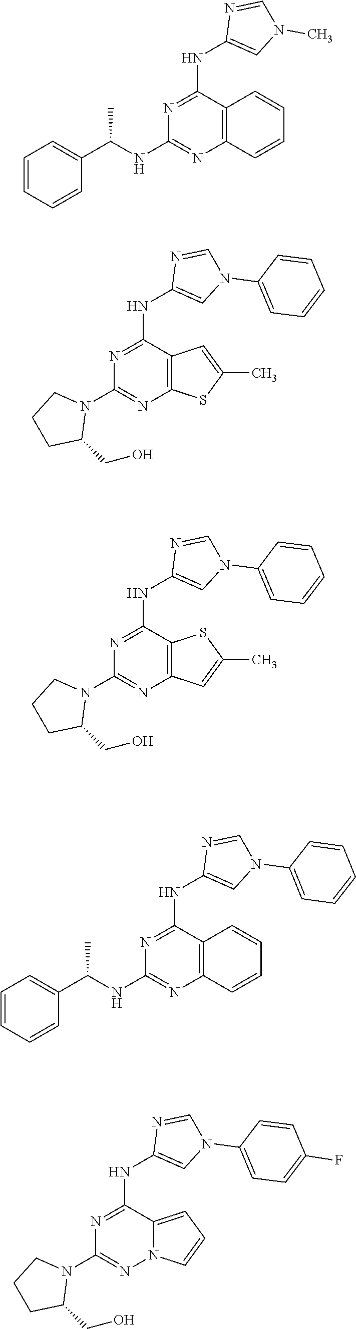 [SMILES: C[C@H](Nc1nc(Nc2cn(C)cn2)c2ccccc2n1)c1ccccc1 ; Cc1cc2c(Nc3cn(-c4ccccc4)cn3)nc(N3CCC[C@H]3CO)nc2s1 ; Cc1cc2nc(N3CCC[C@H]3CO)nc(Nc3cn(-c4ccccc4)cn3)c2s1 ; C[C@H](Nc1nc(Nc2cn(-c3ccccc3)cn2)c2ccccc2n1)c1ccccc1 ; OC[C@@H]1CCCN1c1nc(Nc2cn(-c3ccc(F)cc3)cn2)c2cccn2n1] 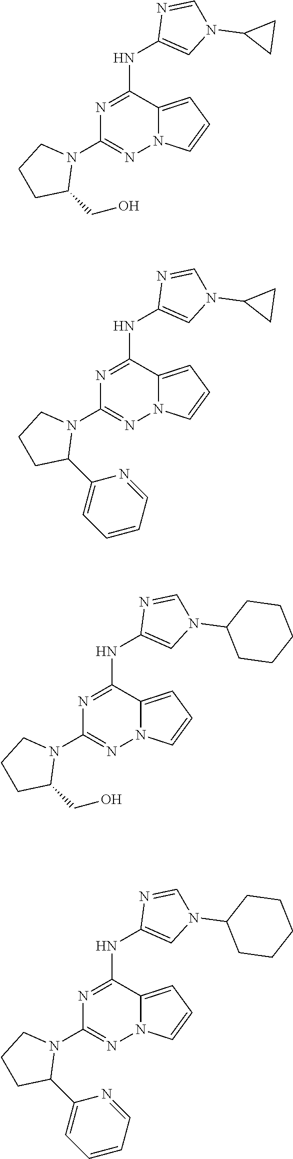 [SMILES: OC[C@@H]1CCCN1c1nc(Nc2cn(C3CC3)cn2)c2cccn2n1 ; c1ccc(C2CCCN2c2nc(Nc3cn(C4CC4)cn3)c3cccn3n2)nc1 ; OC[C@@H]1CCCN1c1nc(Nc2cn(C3CCCCC3)cn2)c2cccn2n1 ; c1ccc(C2CCCN2c2nc(Nc3cn(C4CCCCC4)cn3)c3cccn3n2)nc1] 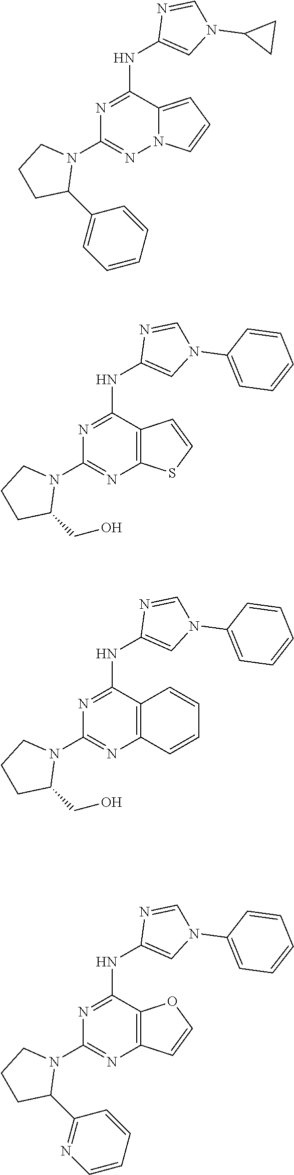 [SMILES: c1ccc(C2CCCN2c2nc(Nc3cn(C4CC4)cn3)c3cccn3n2)cc1 ; OC[C@@H]1CCCN1c1nc(Nc2cn(-c3ccccc3)cn2)c2ccsc2n1 ; OC[C@@H]1CCCN1c1nc(Nc2cn(-c3ccccc3)cn2)c2ccccc2n1 ; c1ccc(-n2cnc(Nc3nc(N4CCCC4c4ccccn4)nc4ccoc34)c2)cc1] 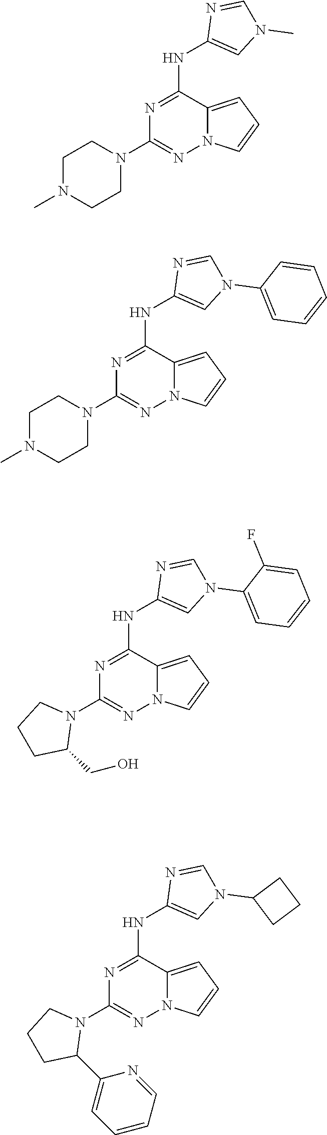 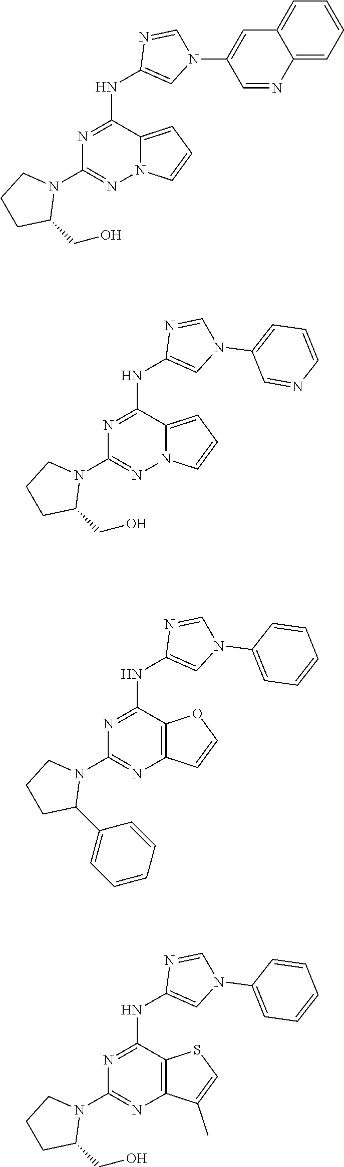 [SMILES: OC[C@@H]1CCCN1c1nc(Nc2cn(-c3cnc4ccccc4c3)cn2)c2cccn2n1 ; OC[C@@H]1CCCN1c1nc(Nc2cn(-c3cccnc3)cn2)c2cccn2n1 ; c1ccc(C2CCCN2c2nc(Nc3cn(-c4ccccc4)cn3)c3occc3n2)cc1 ; Cc1csc2c(Nc3cn(-c4ccccc4)cn3)nc(N3CCC[C@H]3CO)nc12] 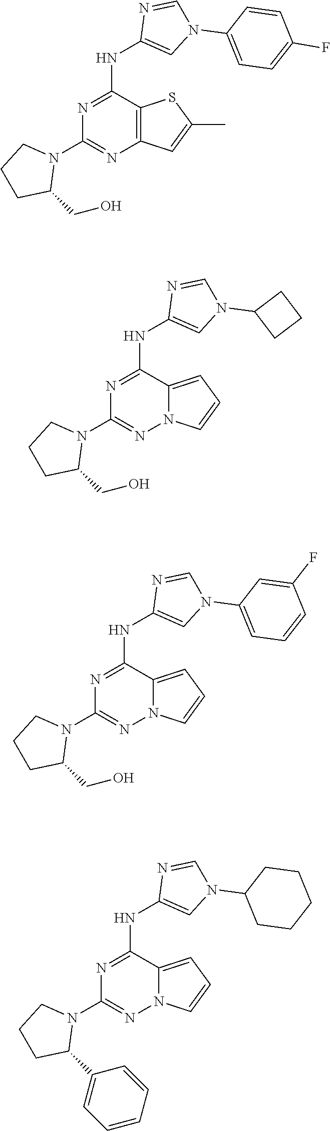 [SMILES: Cc1cc2nc(N3CCC[C@H]3CO)nc(Nc3cn(-c4ccc(F)cc4)cn3)c2s1 ; OC[C@@H]1CCCN1c1nc(Nc2cn(C3CCC3)cn2)c2cccn2n1 ; OC[C@@H]1CCCN1c1nc(Nc2cn(-c3cccc(F)c3)cn2)c2cccn2n1 ; c1ccc([C@@H]2CCCN2c2nc(Nc3cn(C4CCCCC4)cn3)c3cccn3n2)cc1] 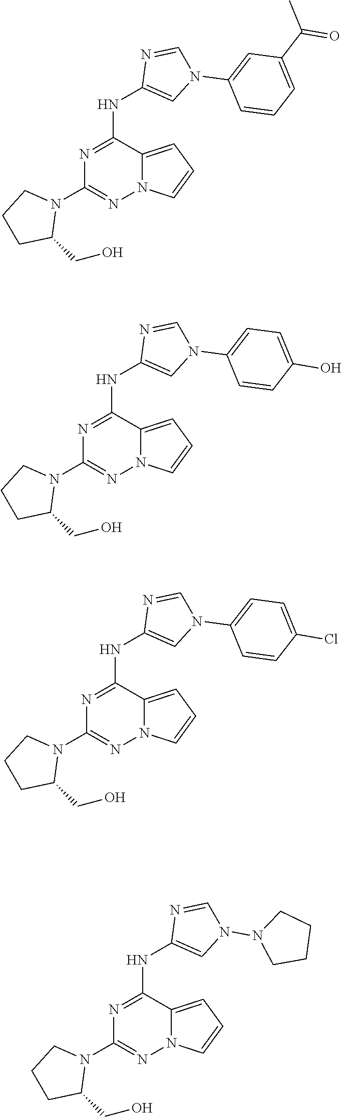 [SMILES: CC(=O)c1cccc(-n2cnc(Nc3nc(N4CCC[C@H]4CO)nn4cccc34)c2)c1 ; OC[C@@H]1CCCN1c1nc(Nc2cn(-c3ccc(O)cc3)cn2)c2cccn2n1 ; OC[C@@H]1CCCN1c1nc(Nc2cn(-c3ccc(Cl)cc3)cn2)c2cccn2n1 ; OC[C@@H]1CCCN1c1nc(Nc2cn(N3CCCC3)cn2)c2cccn2n1] 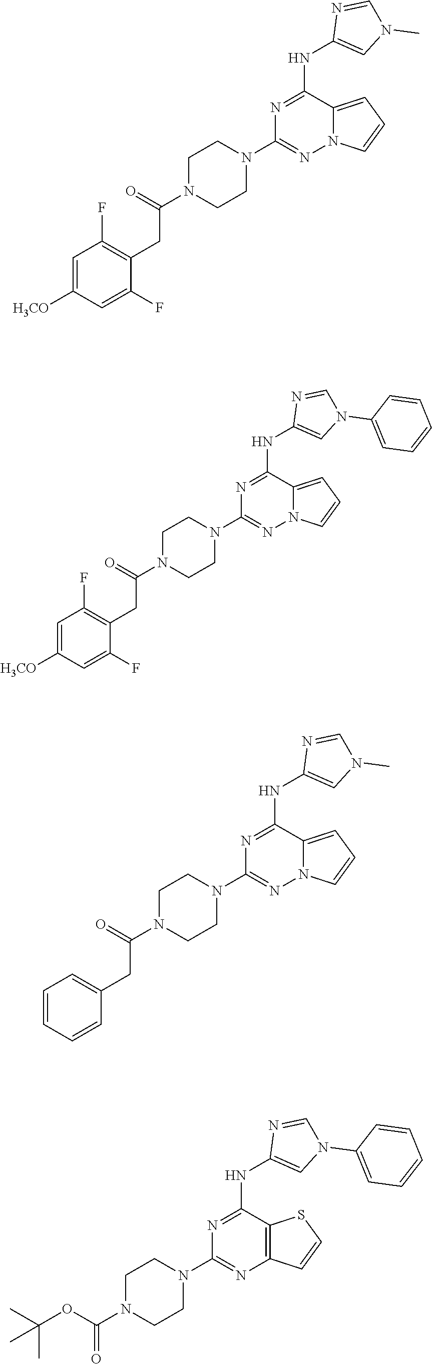 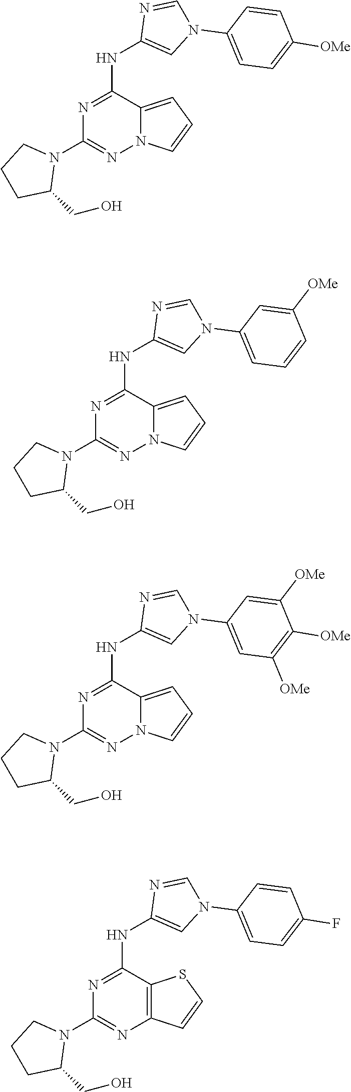 [SMILES: COc1ccc(-n2cnc(Nc3nc(N4CCC[C@H]4CO)nn4cccc34)c2)cc1 ; COc1cccc(-n2cnc(Nc3nc(N4CCC[C@H]4CO)nn4cccc34)c2)c1 ; COc1cc(-n2cnc(Nc3nc(N4CCC[C@H]4CO)nn4cccc34)c2)cc(OC)c1OC ; OC[C@@H]1CCCN1c1nc(Nc2cn(-c3ccc(F)cc3)cn2)c2sccc2n1] 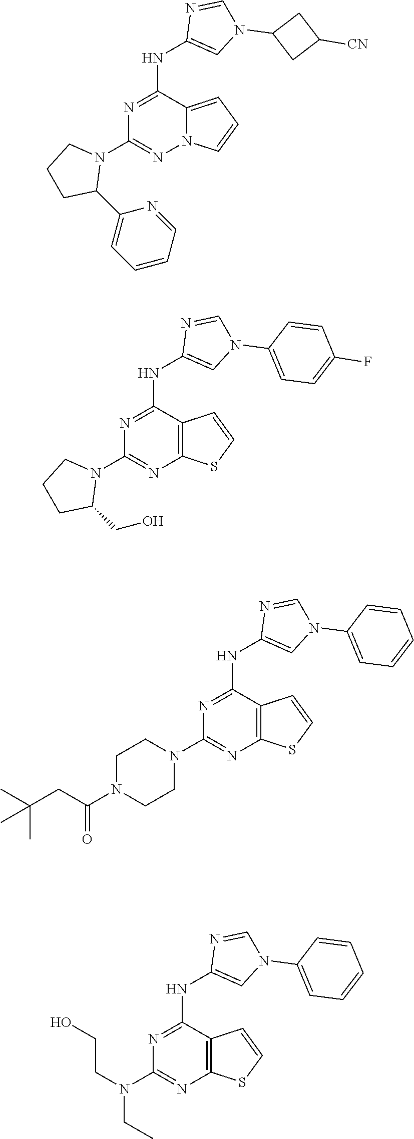 [SMILES: N#CC1CC(n2cnc(Nc3nc(N4CCCC4c4ccccn4)nn4cccc34)c2)C1 ; OC[C@@H]1CCCN1c1nc(Nc2cn(-c3ccc(F)cc3)cn2)c2ccsc2n1 ; CC(C)(C)CC(=O)N1CCN(c2nc(Nc3cn(-c4ccccc4)cn3)c3ccsc3n2)CC1 ; CCN(CCO)c1nc(Nc2cn(-c3ccccc3)cn2)c2ccsc2n1] 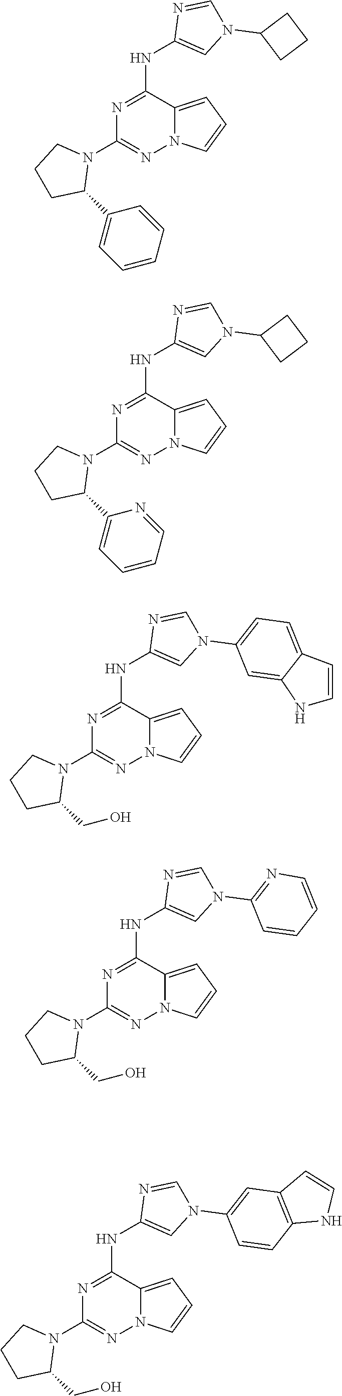 [SMILES: c1ccc([C@@H]2CCCN2c2nc(Nc3cn(C4CCC4)cn3)c3cccn3n2)cc1 ; c1ccc([C@@H]2CCCN2c2nc(Nc3cn(C4CCC4)cn3)c3cccn3n2)nc1 ; OC[C@@H]1CCCN1c1nc(Nc2cn(-c3ccc4cc[nH]c4c3)cn2)c2cccn2n1 ; OC[C@@H]1CCCN1c1nc(Nc2cn(-c3ccccn3)cn2)c2cccn2n1 ; OC[C@@H]1CCCN1c1nc(Nc2cn(-c3ccc4[nH]ccc4c3)cn2)c2cccn2n1] 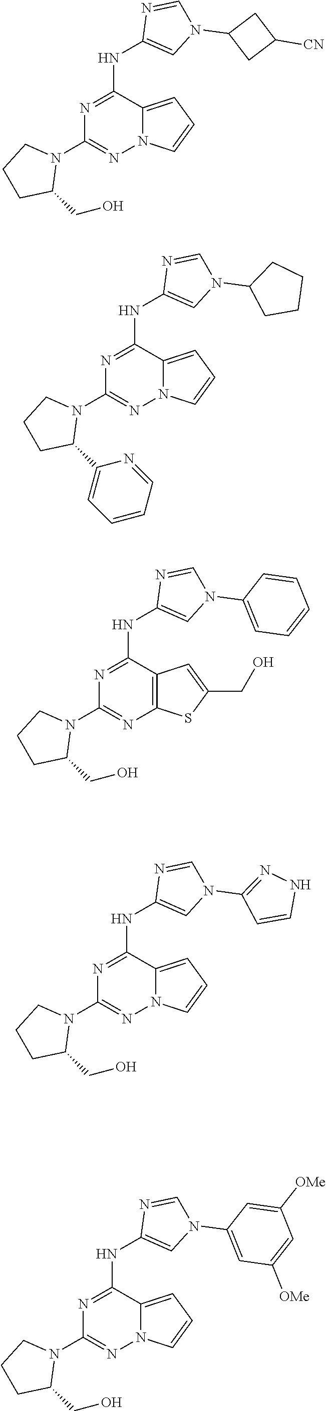 [SMILES: N#CC1CC(n2cnc(Nc3nc(N4CCC[C@H]4CO)nn4cccc34)c2)C1 ; c1ccc([C@@H]2CCCN2c2nc(Nc3cn(C4CCCC4)cn3)c3cccn3n2)nc1 ; OCc1cc2c(Nc3cn(-c4ccccc4)cn3)nc(N3CCC[C@H]3CO)nc2s1 ; OC[C@@H]1CCCN1c1nc(Nc2cn(-c3cc[nH]n3)cn2)c2cccn2n1 ; COc1cc(OC)cc(-n2cnc(Nc3nc(N4CCC[C@H]4CO)nn4cccc34)c2)c1] 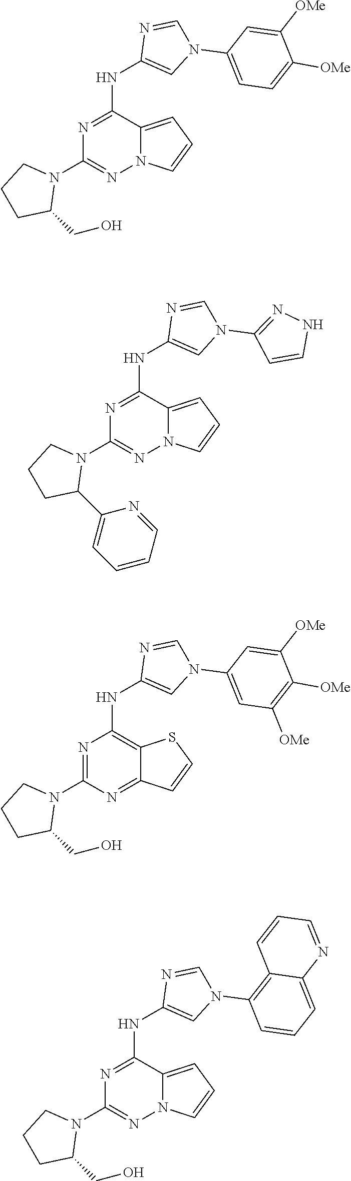 [SMILES: COc1ccc(-n2cnc(Nc3nc(N4CCC[C@H]4CO)nn4cccc34)c2)cc1OC ; c1ccc(C2CCCN2c2nc(Nc3cn(-c4cc[nH]n4)cn3)c3cccn3n2)nc1 ; COc1cc(-n2cnc(Nc3nc(N4CCC[C@H]4CO)nc4ccsc34)c2)cc(OC)c1OC ; OC[C@@H]1CCCN1c1nc(Nc2cn(-c3cccc4ncccc34)cn2)c2cccn2n1] 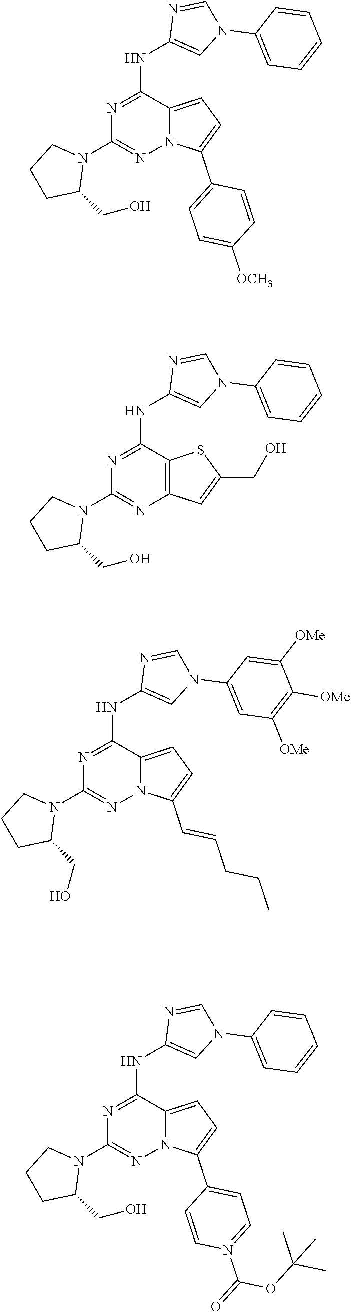 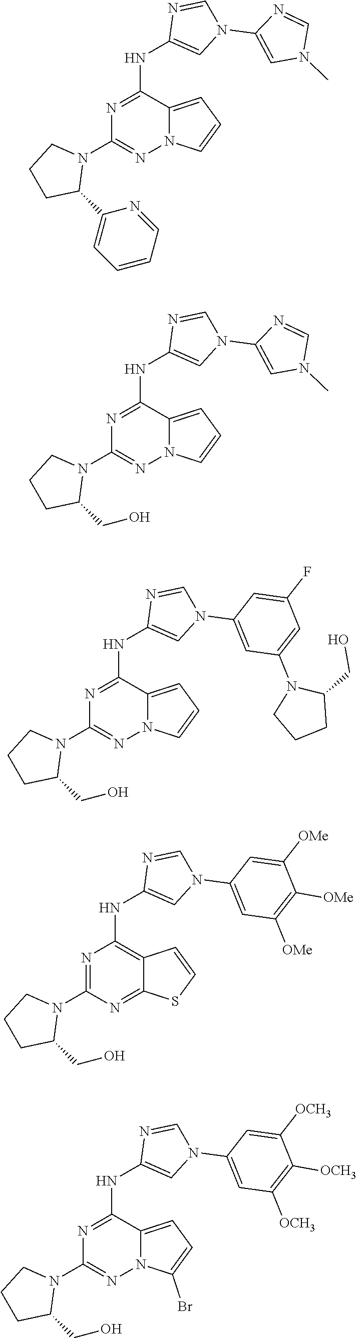 [SMILES: Cn1cnc(-n2cnc(Nc3nc(N4CCC[C@H]4c4ccccn4)nn4cccc34)c2)c1 ; Cn1cnc(-n2cnc(Nc3nc(N4CCC[C@H]4CO)nn4cccc34)c2)c1 ; OC[C@@H]1CCCN1c1cc(F)cc(-n2cnc(Nc3nc(N4CCC[C@H]4CO)nn4cccc34)c2)c1 ; COc1cc(-n2cnc(Nc3nc(N4CCC[C@H]4CO)nc4sccc34)c2)cc(OC)c1OC ; COc1cc(-n2cnc(Nc3nc(N4CCC[C@H]4CO)nn4c(Br)ccc34)c2)cc(OC)c1OC] 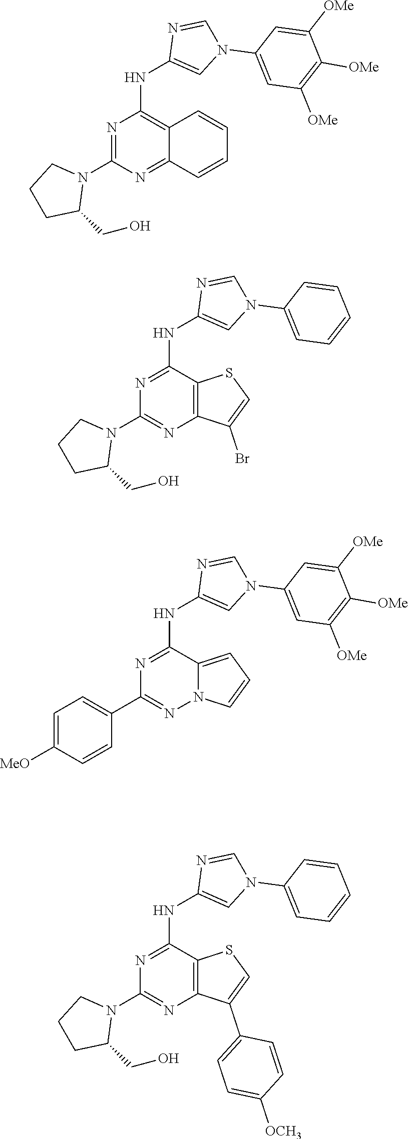 [SMILES: COc1cc(-n2cnc(Nc3nc(N4CCC[C@H]4CO)nc4ccccc34)c2)cc(OC)c1OC ; OC[C@@H]1CCCN1c1nc(Nc2cn(-c3ccccc3)cn2)c2scc(Br)c2n1 ; COc1ccc(-c2nc(Nc3cn(-c4cc(OC)c(OC)c(OC)c4)cn3)c3cccn3n2)cc1 ; COc1ccc(-c2csc3c(Nc4cn(-c5ccccc5)cn4)nc(N4CCC[C@H]4CO)nc23)cc1] 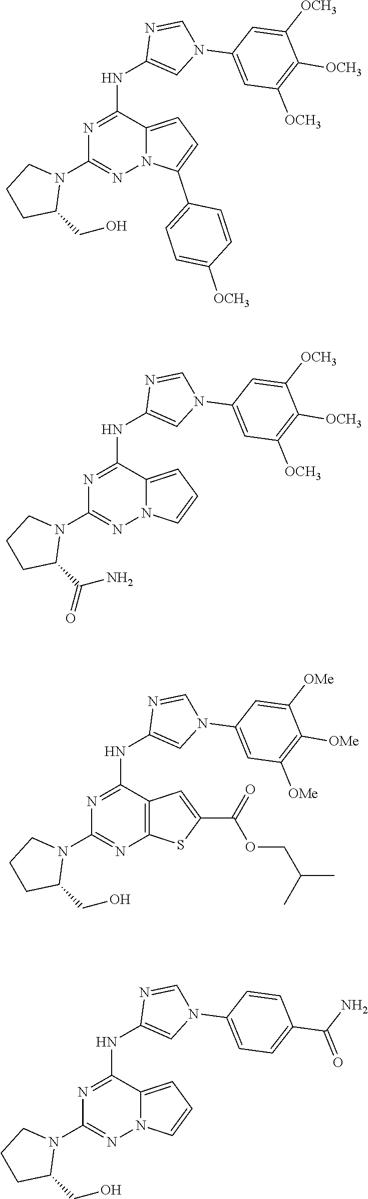 [SMILES: COc1ccc(-c2ccc3c(Nc4cn(-c5cc(OC)c(OC)c(OC)c5)cn4)nc(N4CCC[C@H]4CO)nn23)cc1 ; COc1cc(-n2cnc(Nc3nc(N4CCC[C@H]4C(N)=O)nn4cccc34)c2)cc(OC)c1OC ; COc1cc(-n2cnc(Nc3nc(N4CCC[C@H]4CO)nc4sc(C(=O)OCC(C)C)cc34)c2)cc(OC)c1OC ; NC(=O)c1ccc(-n2cnc(Nc3nc(N4CCC[C@H]4CO)nn4cccc34)c2)cc1] 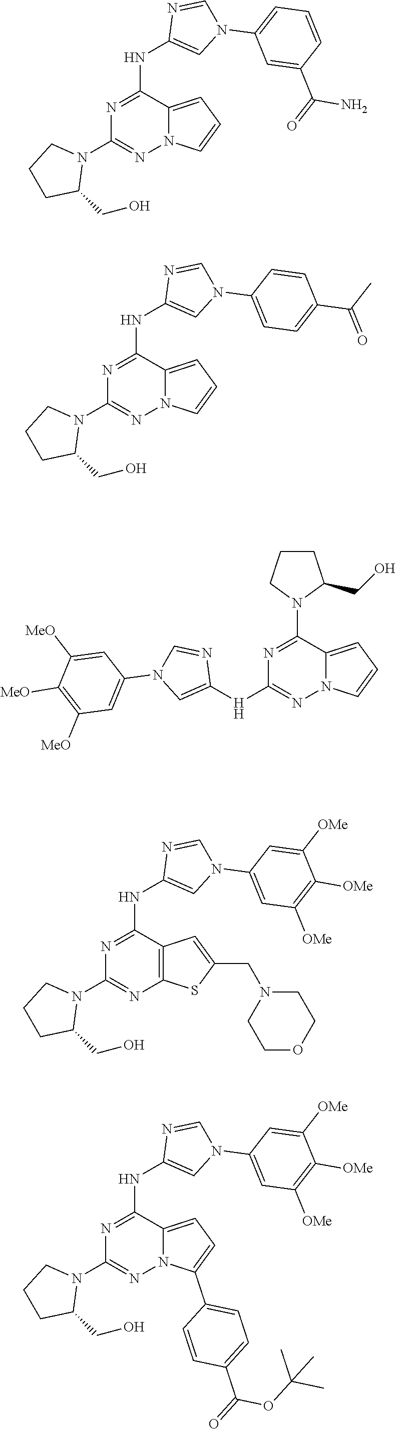 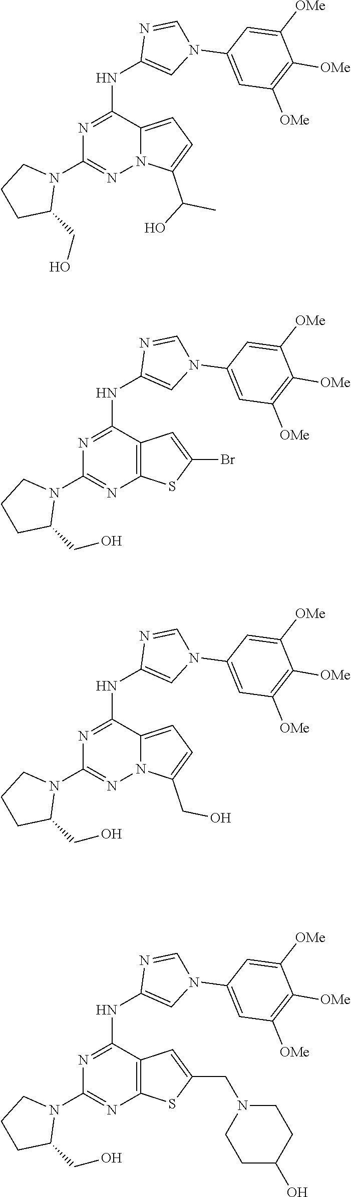 [SMILES: COc1cc(-n2cnc(Nc3nc(N4CCC[C@H]4CO)nn4c(C(C)O)ccc34)c2)cc(OC)c1OC ; COc1cc(-n2cnc(Nc3nc(N4CCC[C@H]4CO)nc4sc(Br)cc34)c2)cc(OC)c1OC ; COc1cc(-n2cnc(Nc3nc(N4CCC[C@H]4CO)nn4c(CO)ccc34)c2)cc(OC)c1OC ; COc1cc(-n2cnc(Nc3nc(N4CCC[C@H]4CO)nc4sc(CN5CCC(O)CC5)cc34)c2)cc(OC)c1OC] 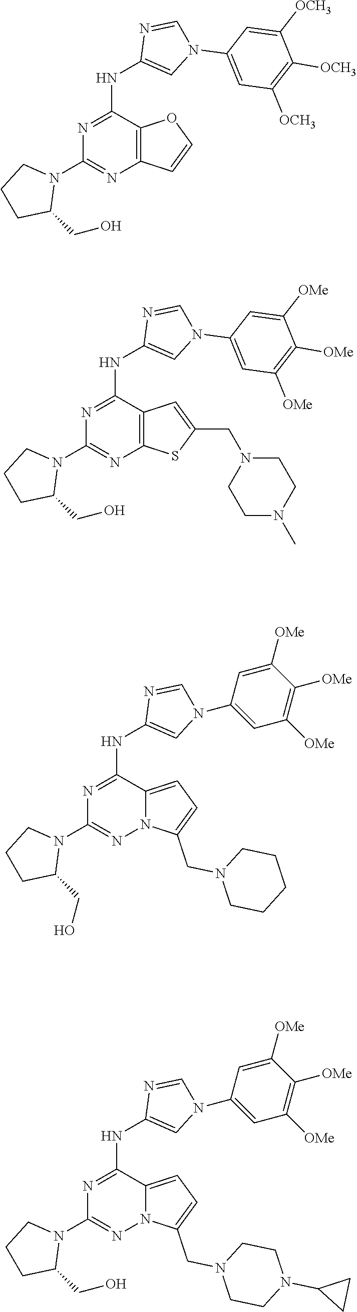 [SMILES: COc1cc(-n2cnc(Nc3nc(N4CCC[C@H]4CO)nc4ccoc34)c2)cc(OC)c1OC ; COc1cc(-n2cnc(Nc3nc(N4CCC[C@H]4CO)nc4sc(CN5CCN(C)CC5)cc34)c2)cc(OC)c1OC ; COc1cc(-n2cnc(Nc3nc(N4CCC[C@H]4CO)nn4c(CN5CCCCC5)ccc34)c2)cc(OC)c1OC ; COc1cc(-n2cnc(Nc3nc(N4CCC[C@H]4CO)nn4c(CN5CCN(C6CC6)CC5)ccc34)c2)cc(OC)c1OC] 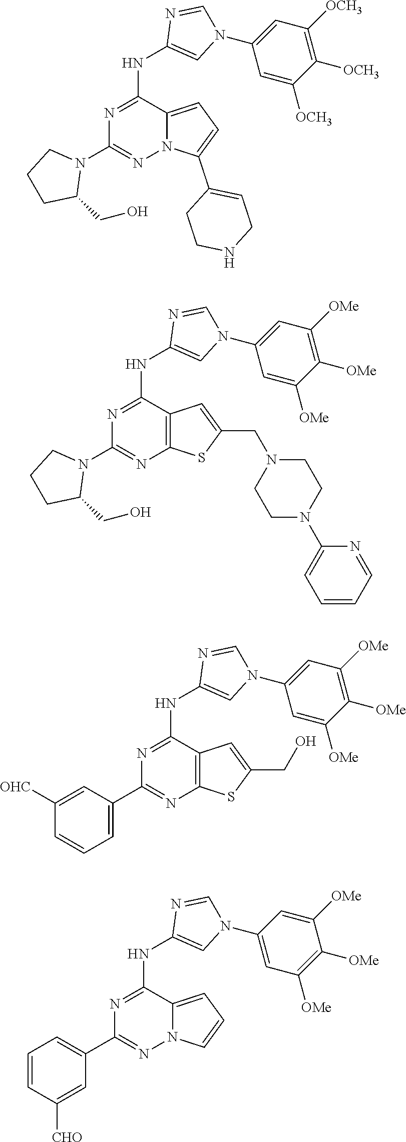 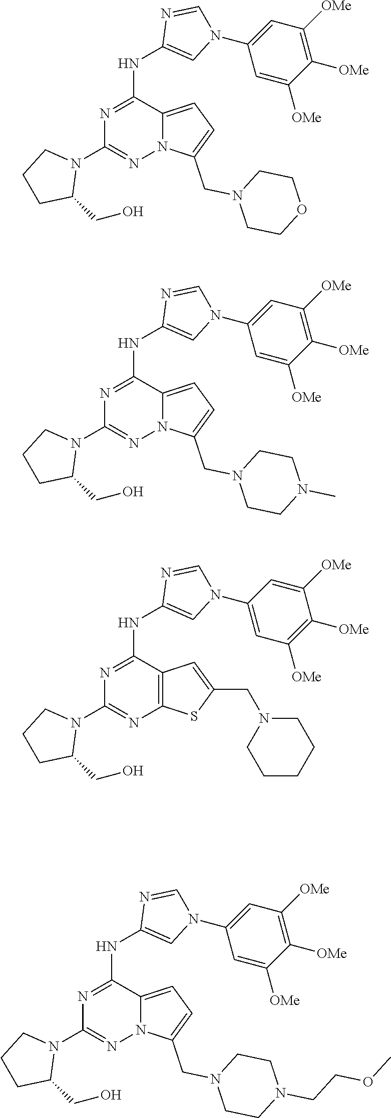 [SMILES: COc1cc(-n2cnc(Nc3nc(N4CCC[C@H]4CO)nn4c(CN5CCOCC5)ccc34)c2)cc(OC)c1OC ; COc1cc(-n2cnc(Nc3nc(N4CCC[C@H]4CO)nn4c(CN5CCN(C)CC5)ccc34)c2)cc(OC)c1OC ; COc1cc(-n2cnc(Nc3nc(N4CCC[C@H]4CO)nc4sc(CN5CCCCC5)cc34)c2)cc(OC)c1OC ; COCCN1CCN(Cc2ccc3c(Nc4cn(-c5cc(OC)c(OC)c(OC)c5)cn4)nc(N4CCC[C@H]4CO)nn23)CC1] 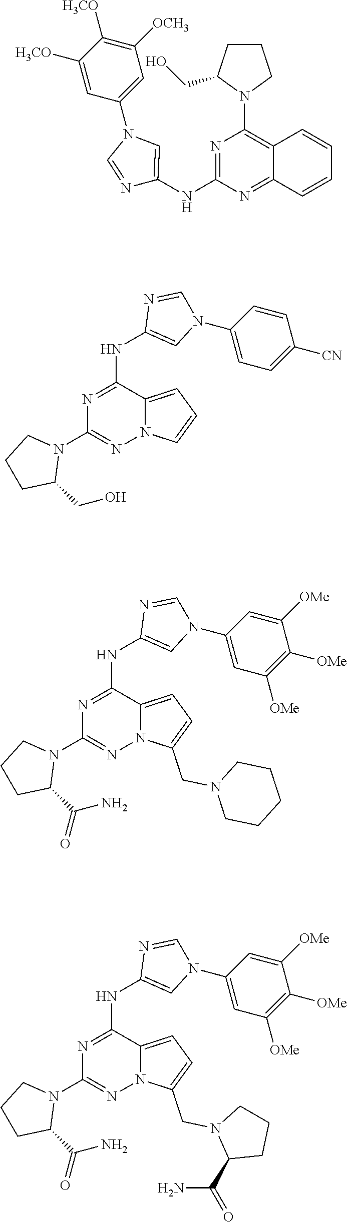 [SMILES: COc1cc(-n2cnc(Nc3nc(N4CCC[C@H]4CO)c4ccccc4n3)c2)cc(OC)c1OC ; N#Cc1ccc(-n2cnc(Nc3nc(N4CCC[C@H]4CO)nn4cccc34)c2)cc1 ; COc1cc(-n2cnc(Nc3nc(N4CCC[C@H]4C(N)=O)nn4c(CN5CCCCC5)ccc34)c2)cc(OC)c1OC ; COc1cc(-n2cnc(Nc3nc(N4CCC[C@H]4C(N)=O)nn4c(CN5CCC[C@H]5C(N)=O)ccc34)c2)cc(OC)c1OC] 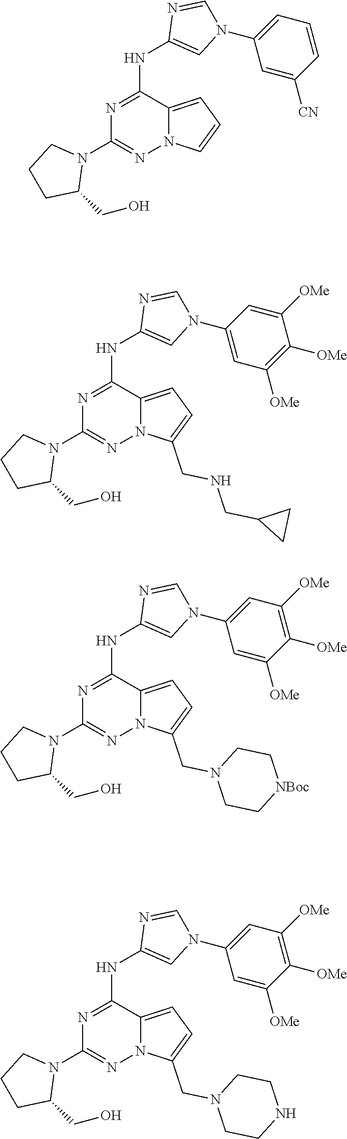 [SMILES: N#Cc1cccc(-n2cnc(Nc3nc(N4CCC[C@H]4CO)nn4cccc34)c2)c1 ; COc1cc(-n2cnc(Nc3nc(N4CCC[C@H]4CO)nn4c(CNCC5CC5)ccc34)c2)cc(OC)c1OC ; COc1cc(-n2cnc(Nc3nc(N4CCC[C@H]4CO)nn4c(CN5CCN(C(=O)OC(C)(C)C)CC5)ccc34)c2)cc(OC)c1OC ; COc1cc(-n2cnc(Nc3nc(N4CCC[C@H]4CO)nn4c(CN5CCNCC5)ccc34)c2)cc(OC)c1OC] 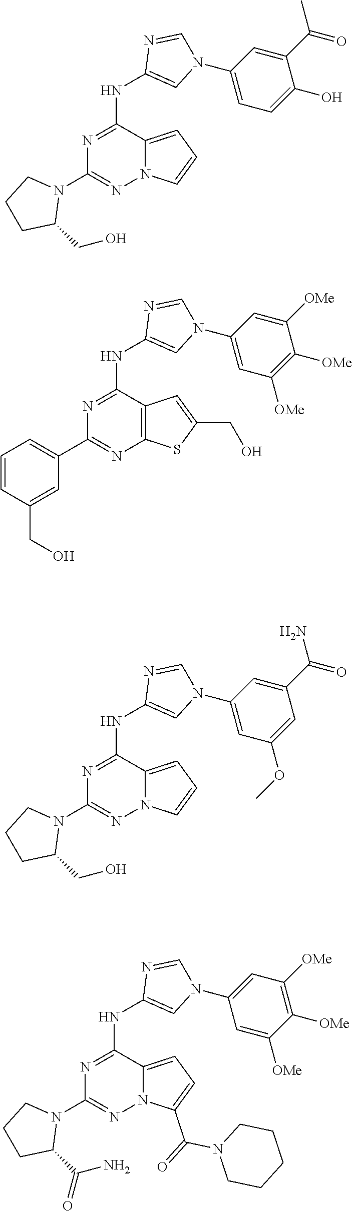 [SMILES: CC(=O)c1cc(-n2cnc(Nc3nc(N4CCC[C@H]4CO)nn4cccc34)c2)ccc1O ; COc1cc(-n2cnc(Nc3nc(-c4cccc(CO)c4)nc4sc(CO)cc34)c2)cc(OC)c1OC ; COc1cc(C(N)=O)cc(-n2cnc(Nc3nc(N4CCC[C@H]4CO)nn4cccc34)c2)c1 ; COc1cc(-n2cnc(Nc3nc(N4CCC[C@H]4C(N)=O)nn4c(C(=O)N5CCCCC5)ccc34)c2)cc(OC)c1OC] 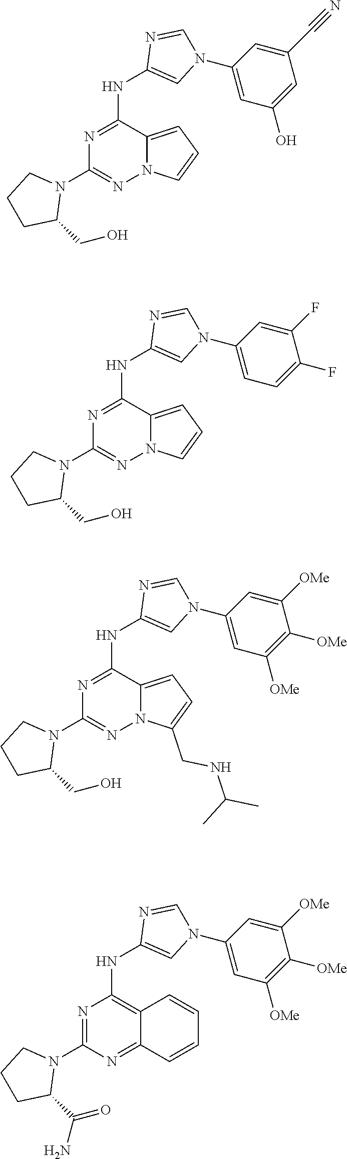 [SMILES: N#Cc1cc(O)cc(-n2cnc(Nc3nc(N4CCC[C@H]4CO)nn4cccc34)c2)c1 ; OC[C@@H]1CCCN1c1nc(Nc2cn(-c3ccc(F)c(F)c3)cn2)c2cccn2n1 ; COc1cc(-n2cnc(Nc3nc(N4CCC[C@H]4CO)nn4c(CNC(C)C)ccc34)c2)cc(OC)c1OC ; COc1cc(-n2cnc(Nc3nc(N4CCC[C@H]4C(N)=O)nc4ccccc34)c2)cc(OC)c1OC] 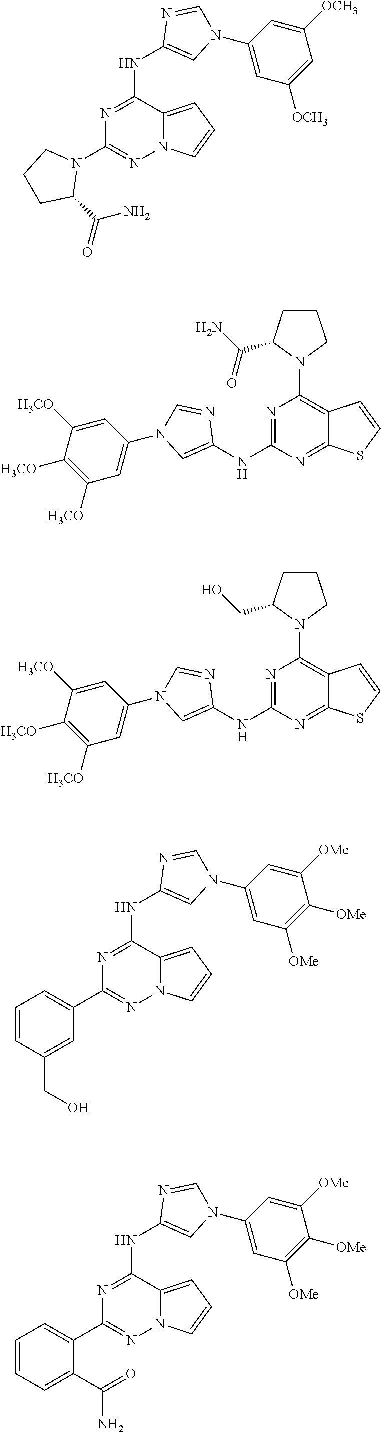 [SMILES: COc1cc(OC)cc(-n2cnc(Nc3nc(N4CCC[C@H]4C(N)=O)nn4cccc34)c2)c1 ; COc1cc(-n2cnc(Nc3nc(N4CCC[C@H]4C(N)=O)c4ccsc4n3)c2)cc(OC)c1OC ; COc1cc(-n2cnc(Nc3nc(N4CCC[C@H]4CO)c4ccsc4n3)c2)cc(OC)c1OC ; COc1cc(-n2cnc(Nc3nc(-c4cccc(CO)c4)nn4cccc34)c2)cc(OC)c1OC ; COc1cc(-n2cnc(Nc3nc(-c4ccccc4C(N)=O)nn4cccc34)c2)cc(OC)c1OC] 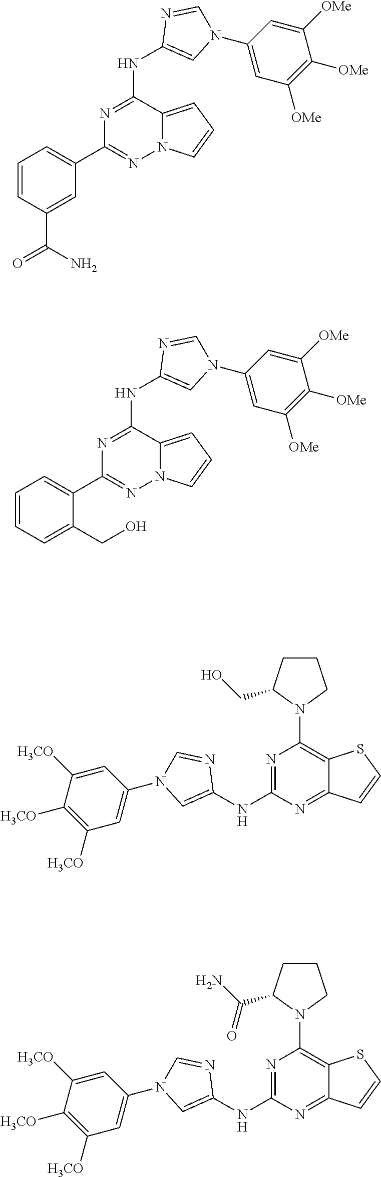 [SMILES: COc1cc(-n2cnc(Nc3nc(-c4cccc(C(N)=O)c4)nn4cccc34)c2)cc(OC)c1OC ; COc1cc(-n2cnc(Nc3nc(-c4ccccc4CO)nn4cccc34)c2)cc(OC)c1OC ; COc1cc(-n2cnc(Nc3nc(N4CCC[C@H]4CO)c4sccc4n3)c2)cc(OC)c1OC ; COc1cc(-n2cnc(Nc3nc(N4CCC[C@H]4C(N)=O)c4sccc4n3)c2)cc(OC)c1OC] 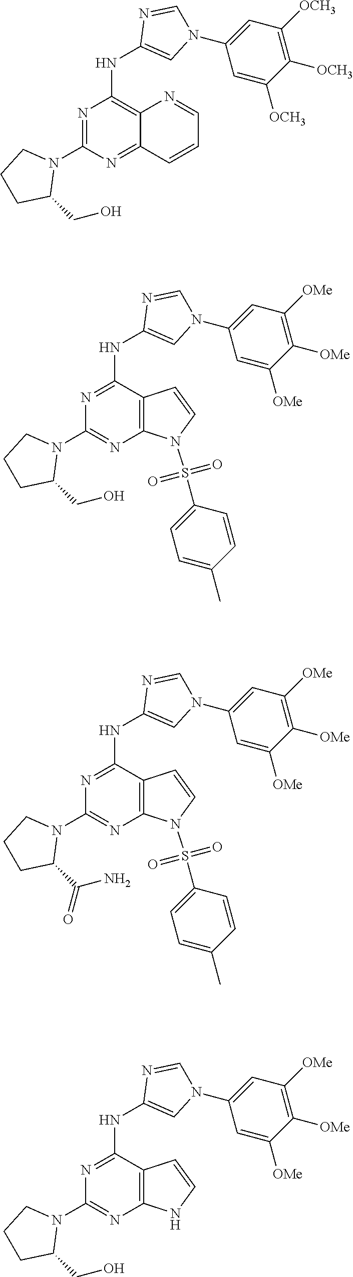 [SMILES: COc1cc(-n2cnc(Nc3nc(N4CCC[C@H]4CO)nc4cccnc34)c2)cc(OC)c1OC ; COc1cc(-n2cnc(Nc3nc(N4CCC[C@H]4CO)nc4c3ccn4S(=O)(=O)c3ccc(C)cc3)c2)cc(OC)c1OC ; COc1cc(-n2cnc(Nc3nc(N4CCC[C@H]4C(N)=O)nc4c3ccn4S(=O)(=O)c3ccc(C)cc3)c2)cc(OC)c1OC ; COc1cc(-n2cnc(Nc3nc(N4CCC[C@H]4CO)nc4[nH]ccc34)c2)cc(OC)c1OC] 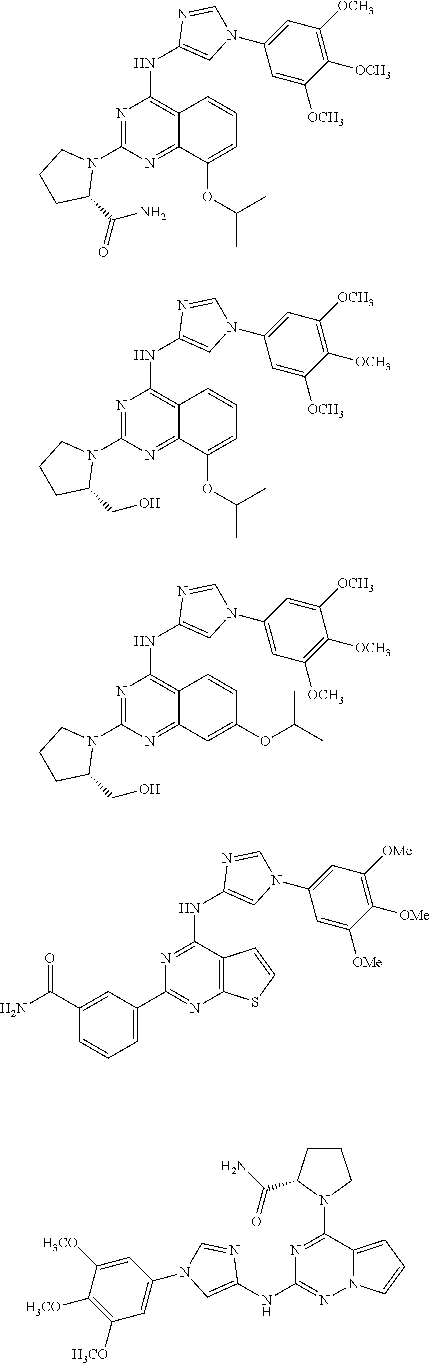 [SMILES: COc1cc(-n2cnc(Nc3nc(N4CCC[C@H]4C(N)=O)nc4c(OC(C)C)cccc34)c2)cc(OC)c1OC ; COc1cc(-n2cnc(Nc3nc(N4CCC[C@H]4CO)nc4c(OC(C)C)cccc34)c2)cc(OC)c1OC ; COc1cc(-n2cnc(Nc3nc(N4CCC[C@H]4CO)nc4cc(OC(C)C)ccc34)c2)cc(OC)c1OC ; COc1cc(-n2cnc(Nc3nc(-c4cccc(C(N)=O)c4)nc4sccc34)c2)cc(OC)c1OC ; COc1cc(-n2cnc(Nc3nc(N4CCC[C@H]4C(N)=O)c4cccn4n3)c2)cc(OC)c1OC] 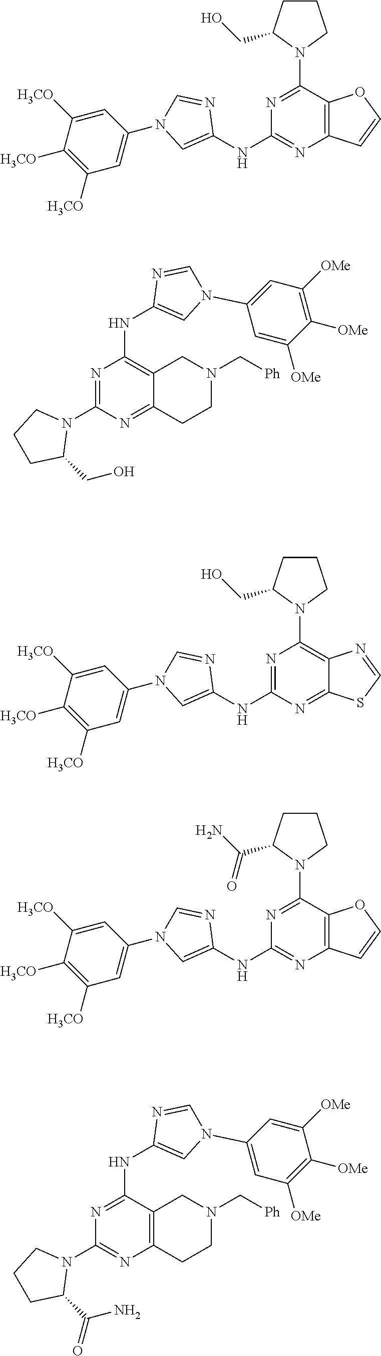 [SMILES: COc1cc(-n2cnc(Nc3nc(N4CCC[C@H]4CO)c4occc4n3)c2)cc(OC)c1OC ; COc1cc(-n2cnc(Nc3nc(N4CCC[C@H]4CO)nc4c3CN(Cc3ccccc3)CC4)c2)cc(OC)c1OC ; COc1cc(-n2cnc(Nc3nc(N4CCC[C@H]4CO)c4ncsc4n3)c2)cc(OC)c1OC ; COc1cc(-n2cnc(Nc3nc(N4CCC[C@H]4C(N)=O)c4occc4n3)c2)cc(OC)c1OC ; COc1cc(-n2cnc(Nc3nc(N4CCC[C@H]4C(N)=O)nc4c3CN(Cc3ccccc3)CC4)c2)cc(OC)c1OC] 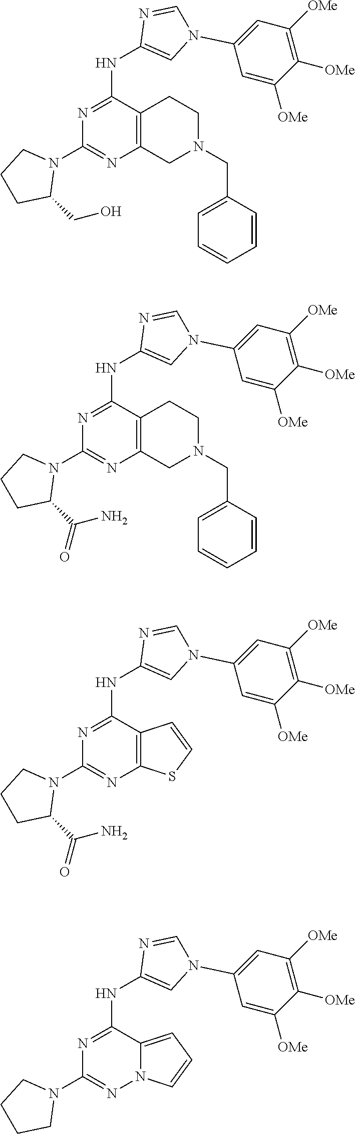 [SMILES: COc1cc(-n2cnc(Nc3nc(N4CCC[C@H]4CO)nc4c3CCN(Cc3ccccc3)C4)c2)cc(OC)c1OC ; COc1cc(-n2cnc(Nc3nc(N4CCC[C@H]4C(N)=O)nc4c3CCN(Cc3ccccc3)C4)c2)cc(OC)c1OC ; COc1cc(-n2cnc(Nc3nc(N4CCC[C@H]4C(N)=O)nc4sccc34)c2)cc(OC)c1OC ; COc1cc(-n2cnc(Nc3nc(N4CCCC4)nn4cccc34)c2)cc(OC)c1OC] 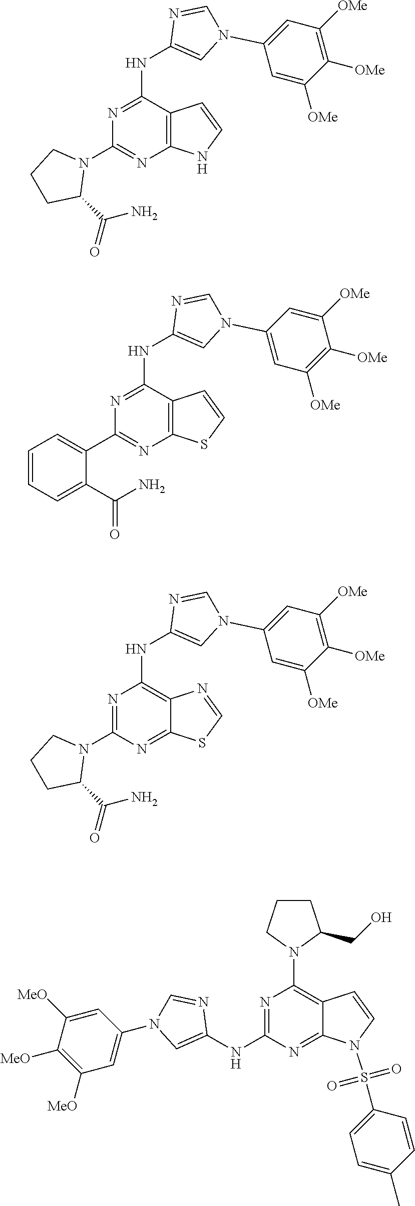 [SMILES: COc1cc(-n2cnc(Nc3nc(N4CCC[C@H]4C(N)=O)nc4[nH]ccc34)c2)cc(OC)c1OC ; COc1cc(-n2cnc(Nc3nc(-c4ccccc4C(N)=O)nc4sccc34)c2)cc(OC)c1OC ; COc1cc(-n2cnc(Nc3nc(N4CCC[C@H]4C(N)=O)nc4scnc34)c2)cc(OC)c1OC ; COc1cc(-n2cnc(Nc3nc(N4CCC[C@H]4CO)c4ccn(S(=O)(=O)c5ccc(C)cc5)c4n3)c2)cc(OC)c1OC] 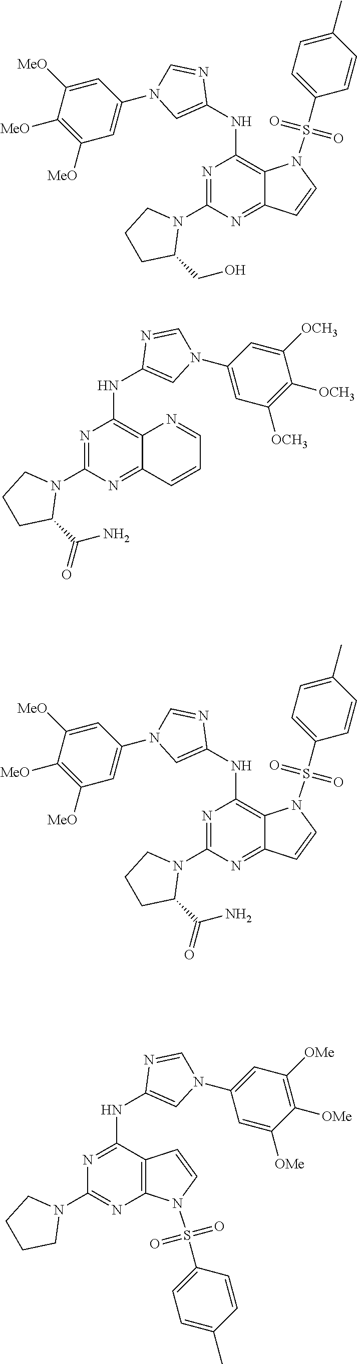 [SMILES: COc1cc(-n2cnc(Nc3nc(N4CCC[C@H]4CO)nc4ccn(S(=O)(=O)c5ccc(C)cc5)c34)c2)cc(OC)c1OC ; COc1cc(-n2cnc(Nc3nc(N4CCC[C@H]4C(N)=O)nc4cccnc34)c2)cc(OC)c1OC ; COc1cc(-n2cnc(Nc3nc(N4CCC[C@H]4C(N)=O)nc4ccn(S(=O)(=O)c5ccc(C)cc5)c34)c2)cc(OC)c1OC ; COc1cc(-n2cnc(Nc3nc(N4CCCC4)nc4c3ccn4S(=O)(=O)c3ccc(C)cc3)c2)cc(OC)c1OC] 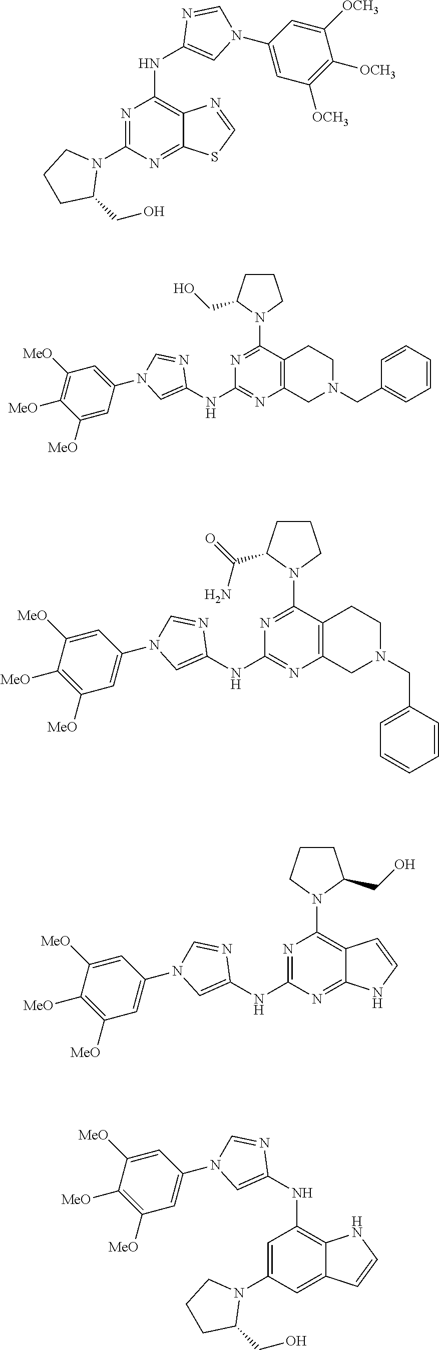 [SMILES: COc1cc(-n2cnc(Nc3nc(N4CCC[C@H]4CO)nc4scnc34)c2)cc(OC)c1OC ; COc1cc(-n2cnc(Nc3nc4c(c(N5CCC[C@H]5CO)n3)CCN(Cc3ccccc3)C4)c2)cc(OC)c1OC ; COc1cc(-n2cnc(Nc3nc4c(c(N5CCC[C@H]5C(N)=O)n3)CCN(Cc3ccccc3)C4)c2)cc(OC)c1OC ; COc1cc(-n2cnc(Nc3nc(N4CCC[C@H]4CO)c4cc[nH]c4n3)c2)cc(OC)c1OC ; COc1cc(-n2cnc(Nc3cc(N4CCC[C@H]4CO)cc4cc[nH]c34)c2)cc(OC)c1OC] 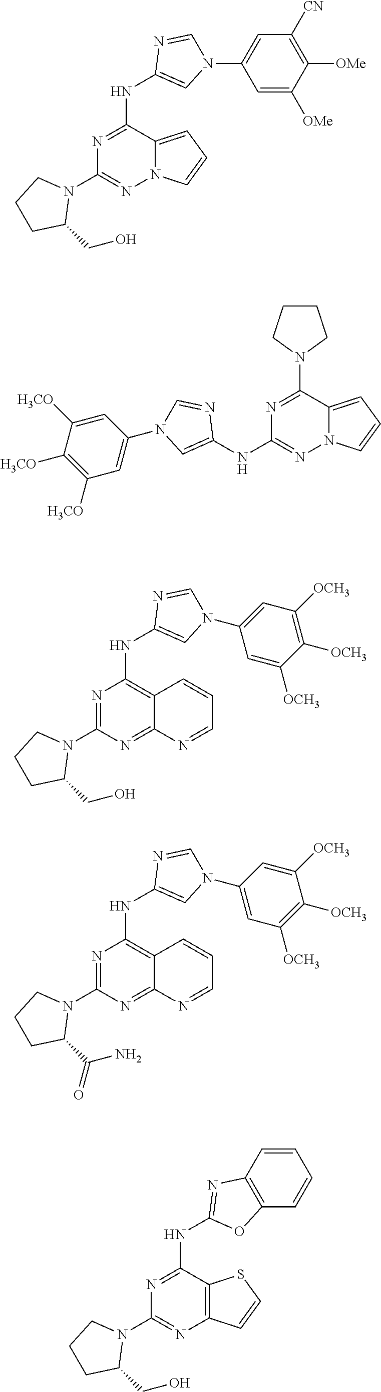 [SMILES: COc1cc(-n2cnc(Nc3nc(N4CCC[C@H]4CO)nn4cccc34)c2)cc(C#N)c1OC ; COc1cc(-n2cnc(Nc3nc(N4CCCC4)c4cccn4n3)c2)cc(OC)c1OC ; COc1cc(-n2cnc(Nc3nc(N4CCC[C@H]4CO)nc4ncccc34)c2)cc(OC)c1OC ; COc1cc(-n2cnc(Nc3nc(N4CCC[C@H]4C(N)=O)nc4ncccc34)c2)cc(OC)c1OC ; OC[C@@H]1CCCN1c1nc(Nc2nc3ccccc3o2)c2sccc2n1] 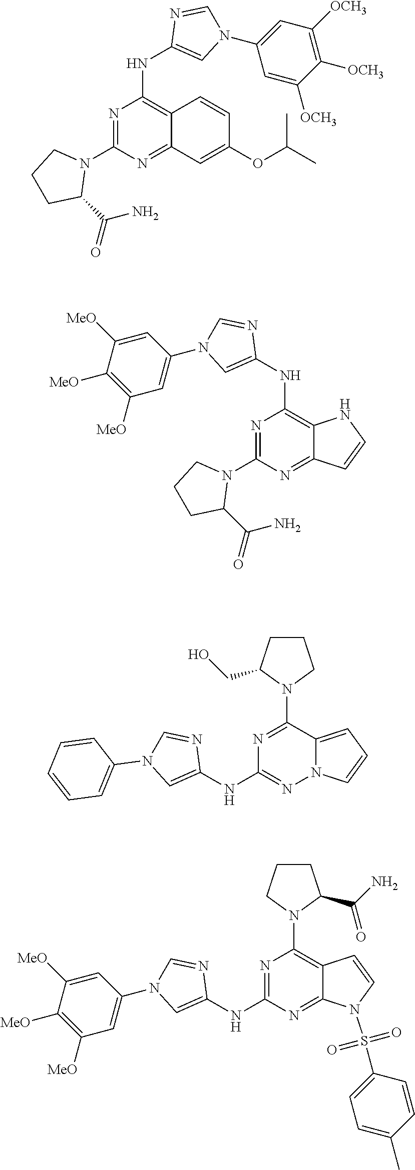 [SMILES: COc1cc(-n2cnc(Nc3nc(N4CCC[C@H]4C(N)=O)nc4cc(OC(C)C)ccc34)c2)cc(OC)c1OC ; COc1cc(-n2cnc(Nc3nc(N4CCCC4C(N)=O)nc4cc[nH]c34)c2)cc(OC)c1OC ; OC[C@@H]1CCCN1c1nc(Nc2cn(-c3ccccc3)cn2)nn2cccc12 ; COc1cc(-n2cnc(Nc3nc(N4CCC[C@H]4C(N)=O)c4ccn(S(=O)(=O)c5ccc(C)cc5)c4n3)c2)cc(OC)c1OC] 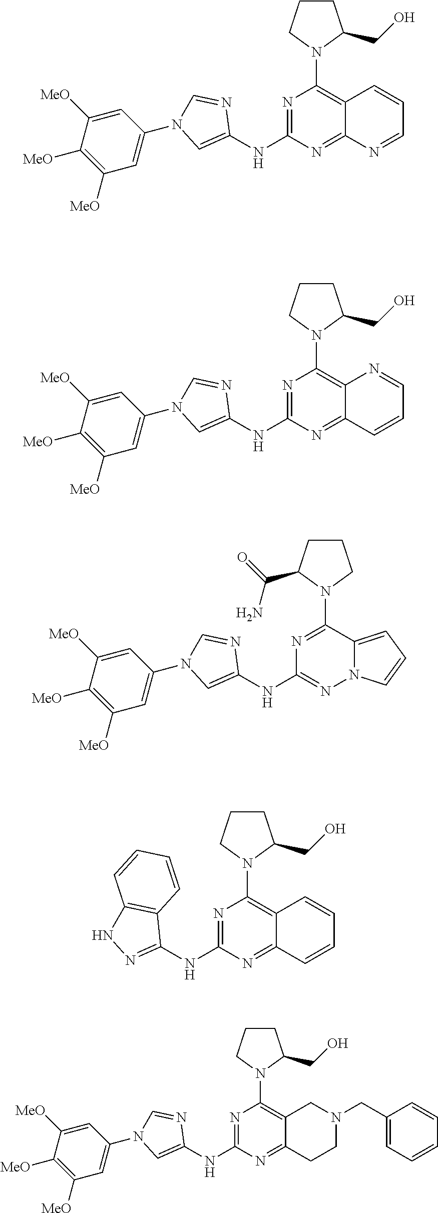 [SMILES: COc1cc(-n2cnc(Nc3nc(N4CCC[C@H]4CO)c4cccnc4n3)c2)cc(OC)c1OC ; COc1cc(-n2cnc(Nc3nc(N4CCC[C@H]4CO)c4ncccc4n3)c2)cc(OC)c1OC ; COc1cc(-n2cnc(Nc3nc(N4CCC[C@@H]4C(N)=O)c4cccn4n3)c2)cc(OC)c1OC ; OC[C@@H]1CCCN1c1nc(Nc2n[nH]c3ccccc23)nc2ccccc12 ; COc1cc(-n2cnc(Nc3nc4c(c(N5CCC[C@H]5CO)n3)CN(Cc3ccccc3)CC4)c2)cc(OC)c1OC] 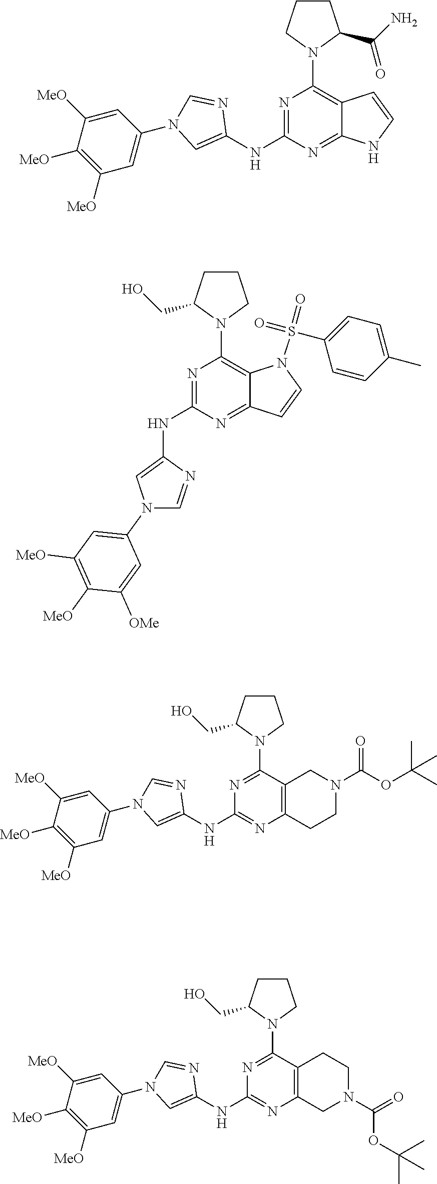 [SMILES: COc1cc(-n2cnc(Nc3nc(N4CCC[C@H]4C(N)=O)c4cc[nH]c4n3)c2)cc(OC)c1OC ; COc1cc(-n2cnc(Nc3nc(N4CCC[C@H]4CO)c4c(ccn4S(=O)(=O)c4ccc(C)cc4)n3)c2)cc(OC)c1OC ; COc1cc(-n2cnc(Nc3nc4c(c(N5CCC[C@H]5CO)n3)CN(C(=O)OC(C)(C)C)CC4)c2)cc(OC)c1OC ; COc1cc(-n2cnc(Nc3nc4c(c(N5CCC[C@H]5CO)n3)CCN(C(=O)OC(C)(C)C)C4)c2)cc(OC)c1OC] 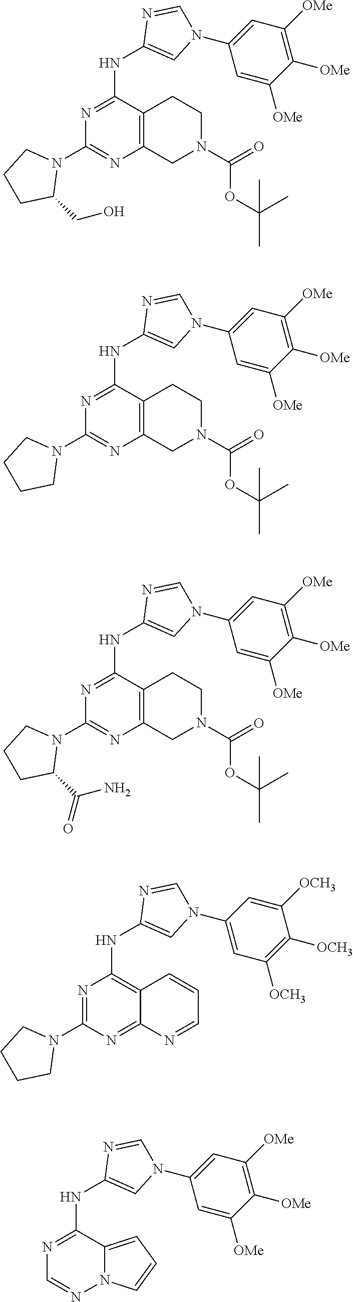 [SMILES: COc1cc(-n2cnc(Nc3nc(N4CCC[C@H]4CO)nc4c3CCN(C(=O)OC(C)(C)C)C4)c2)cc(OC)c1OC ; COc1cc(-n2cnc(Nc3nc(N4CCCC4)nc4c3CCN(C(=O)OC(C)(C)C)C4)c2)cc(OC)c1OC ; COc1cc(-n2cnc(Nc3nc(N4CCC[C@H]4C(N)=O)nc4c3CCN(C(=O)OC(C)(C)C)C4)c2)cc(OC)c1OC ; COc1cc(-n2cnc(Nc3nc(N4CCCC4)nc4ncccc34)c2)cc(OC)c1OC ; COc1cc(-n2cnc(Nc3ncnn4cccc34)c2)cc(OC)c1OC] 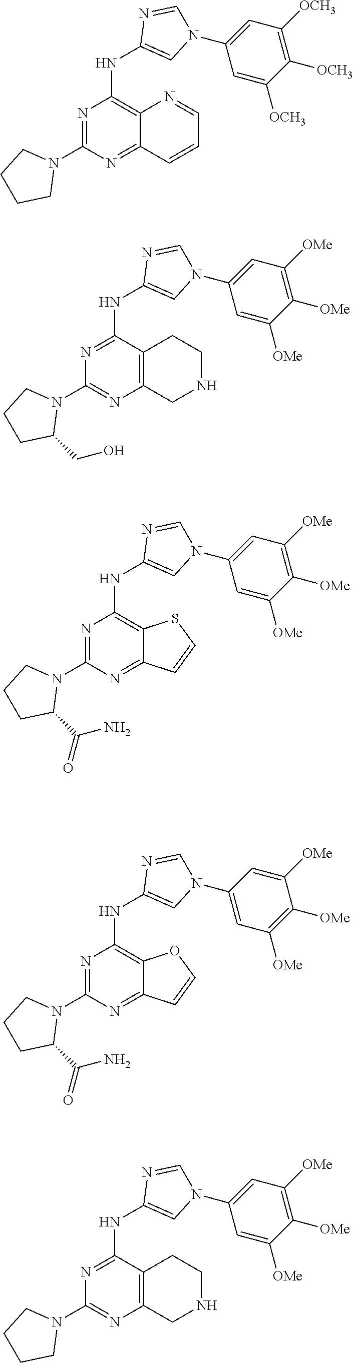 [SMILES: COc1cc(-n2cnc(Nc3nc(N4CCCC4)nc4cccnc34)c2)cc(OC)c1OC ; COc1cc(-n2cnc(Nc3nc(N4CCC[C@H]4CO)nc4c3CCNC4)c2)cc(OC)c1OC ; COc1cc(-n2cnc(Nc3nc(N4CCC[C@H]4C(N)=O)nc4ccsc34)c2)cc(OC)c1OC ; COc1cc(-n2cnc(Nc3nc(N4CCC[C@H]4C(N)=O)nc4ccoc34)c2)cc(OC)c1OC ; COc1cc(-n2cnc(Nc3nc(N4CCCC4)nc4c3CCNC4)c2)cc(OC)c1OC] 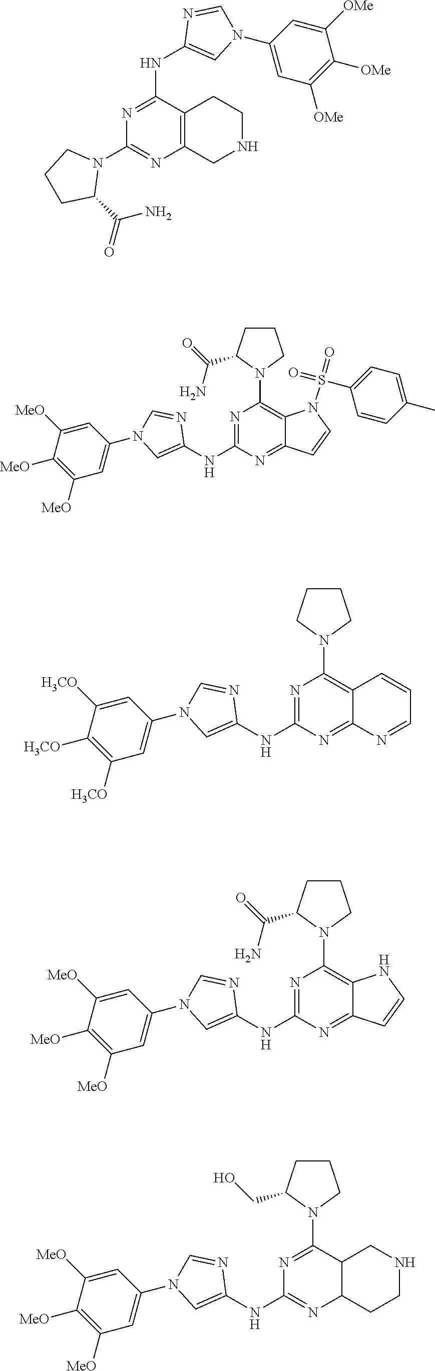 [SMILES: COc1cc(-n2cnc(Nc3nc(N4CCC[C@H]4C(N)=O)nc4c3CCNC4)c2)cc(OC)c1OC ; COc1cc(-n2cnc(Nc3nc(N4CCC[C@H]4C(N)=O)c4c(ccn4S(=O)(=O)c4ccc(C)cc4)n3)c2)cc(OC)c1OC ; COc1cc(-n2cnc(Nc3nc(N4CCCC4)c4cccnc4n3)c2)cc(OC)c1OC ; COc1cc(-n2cnc(Nc3nc(N4CCC[C@H]4C(N)=O)c4[nH]ccc4n3)c2)cc(OC)c1OC ; COc1cc(-n2cnc(NC3=NC4CCNCC4C(N4CCC[C@H]4CO)=N3)c2)cc(OC)c1OC] 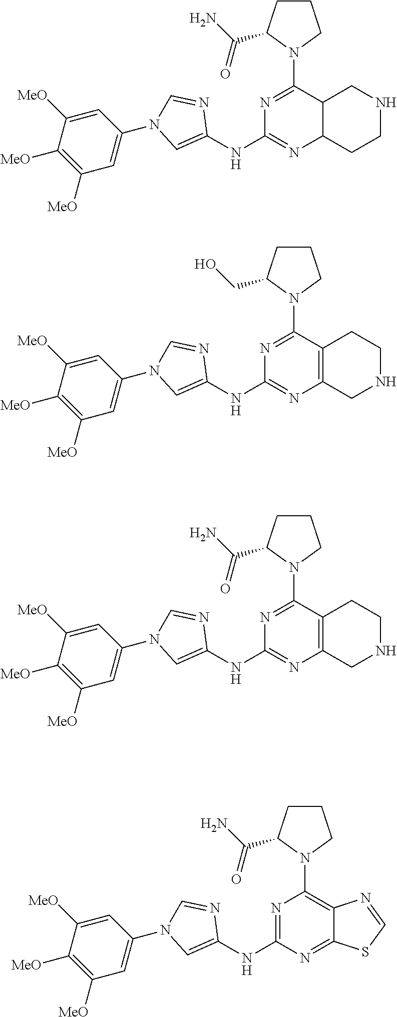 [SMILES: COc1cc(-n2cnc(NC3=NC4CCNCC4C(N4CCC[C@H]4C(N)=O)=N3)c2)cc(OC)c1OC ; COc1cc(-n2cnc(Nc3nc4c(c(N5CCC[C@H]5CO)n3)CCNC4)c2)cc(OC)c1OC ; COc1cc(-n2cnc(Nc3nc4c(c(N5CCC[C@H]5C(N)=O)n3)CCNC4)c2)cc(OC)c1OC ; COc1cc(-n2cnc(Nc3nc(N4CCC[C@H]4C(N)=O)c4ncsc4n3)c2)cc(OC)c1OC] 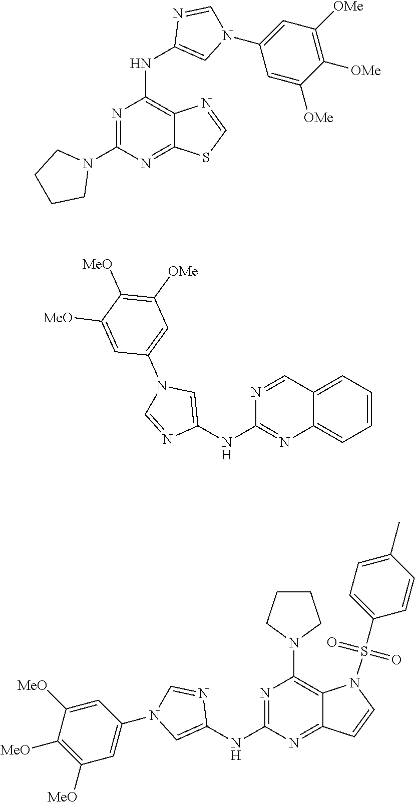 [SMILES: COc1cc(-n2cnc(Nc3nc(N4CCCC4)nc4scnc34)c2)cc(OC)c1OC ; COc1cc(-n2cnc(Nc3ncc4ccccc4n3)c2)cc(OC)c1OC ; COc1cc(-n2cnc(Nc3nc(N4CCCC4)c4c(ccn4S(=O)(=O)c4ccc(C)cc4)n3)c2)cc(OC)c1OC] 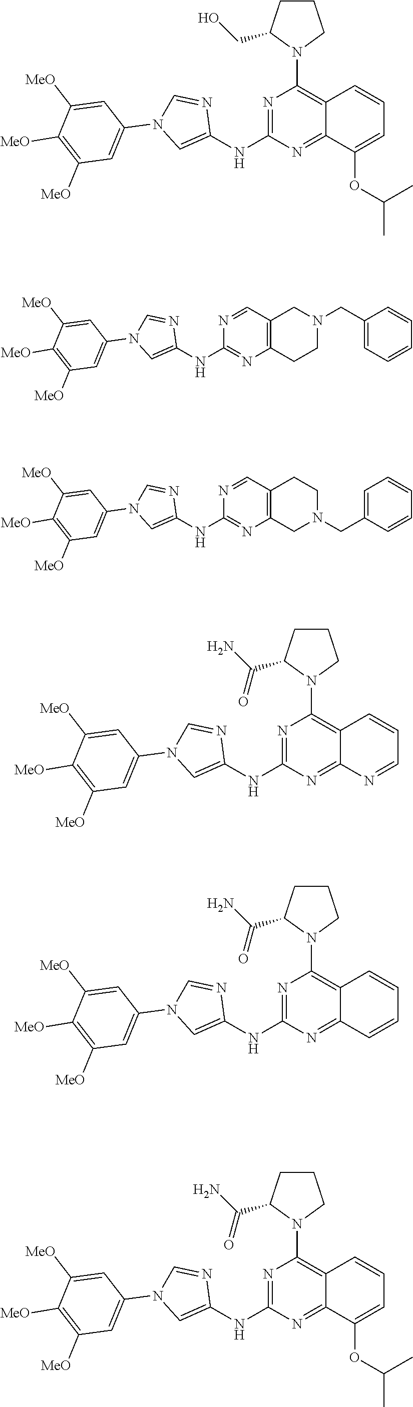 [SMILES: COc1cc(-n2cnc(Nc3nc(N4CCC[C@H]4CO)c4cccc(OC(C)C)c4n3)c2)cc(OC)c1OC ; COc1cc(-n2cnc(Nc3ncc4c(n3)CCN(Cc3ccccc3)C4)c2)cc(OC)c1OC ; COc1cc(-n2cnc(Nc3ncc4c(n3)CN(Cc3ccccc3)CC4)c2)cc(OC)c1OC ; COc1cc(-n2cnc(Nc3nc(N4CCC[C@H]4C(N)=O)c4cccnc4n3)c2)cc(OC)c1OC ; COc1cc(-n2cnc(Nc3nc(N4CCC[C@H]4C(N)=O)c4ccccc4n3)c2)cc(OC)c1OC ; COc1cc(-n2cnc(Nc3nc(N4CCC[C@H]4C(N)=O)c4cccc(OC(C)C)c4n3)c2)cc(OC)c1OC] 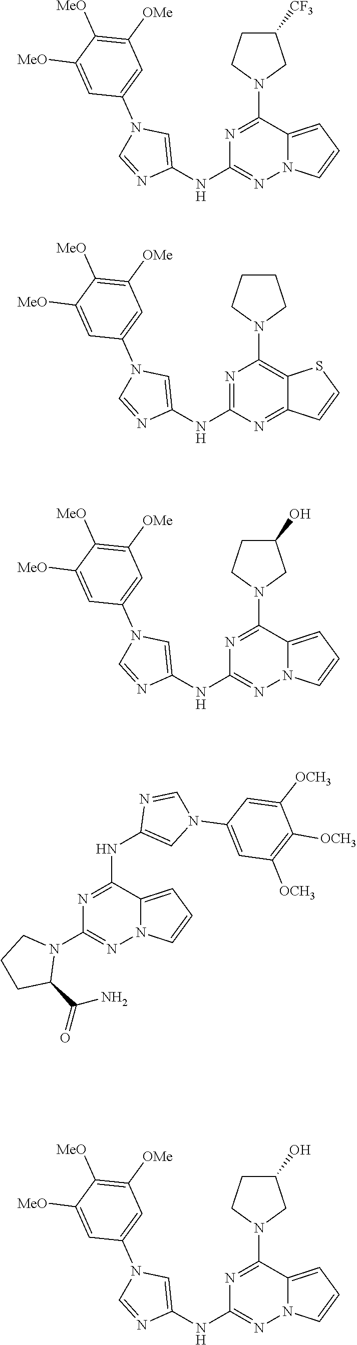 [SMILES: COc1cc(-n2cnc(Nc3nc(N4CC[C@H](C(F)(F)F)C4)c4cccn4n3)c2)cc(OC)c1OC ; COc1cc(-n2cnc(Nc3nc(N4CCCC4)c4sccc4n3)c2)cc(OC)c1OC ; COc1cc(-n2cnc(Nc3nc(N4CC[C@@H](O)C4)c4cccn4n3)c2)cc(OC)c1OC ; COc1cc(-n2cnc(Nc3nc(N4CCC[C@@H]4C(N)=O)nn4cccc34)c2)cc(OC)c1OC ; COc1cc(-n2cnc(Nc3nc(N4CC[C@H](O)C4)c4cccn4n3)c2)cc(OC)c1OC] 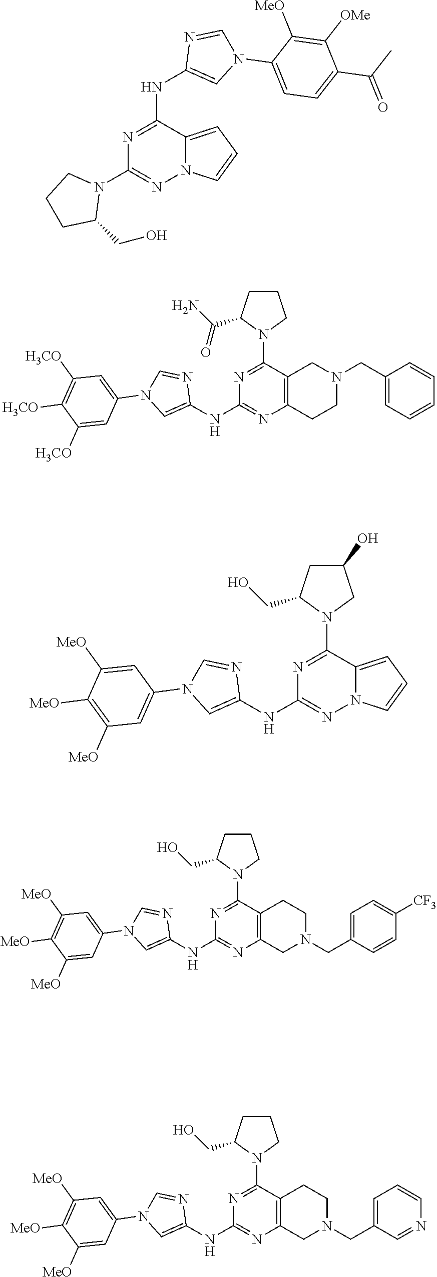 [SMILES: COc1c(C(C)=O)ccc(-n2cnc(Nc3nc(N4CCC[C@H]4CO)nn4cccc34)c2)c1OC ; COc1cc(-n2cnc(Nc3nc4c(c(N5CCC[C@H]5C(N)=O)n3)CN(Cc3ccccc3)CC4)c2)cc(OC)c1OC ; COc1cc(-n2cnc(Nc3nc(N4C[C@H](O)C[C@H]4CO)c4cccn4n3)c2)cc(OC)c1OC ; COc1cc(-n2cnc(Nc3nc4c(c(N5CCC[C@H]5CO)n3)CCN(Cc3ccc(C(F)(F)F)cc3)C4)c2)cc(OC)c1OC ; COc1cc(-n2cnc(Nc3nc4c(c(N5CCC[C@H]5CO)n3)CCN(Cc3cccnc3)C4)c2)cc(OC)c1OC] 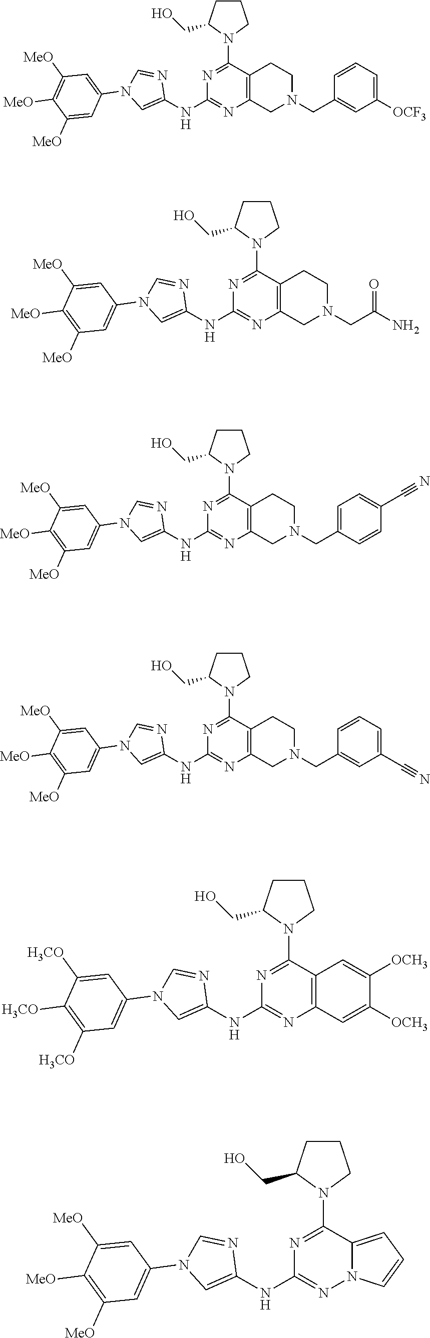 [SMILES: COc1cc(-n2cnc(Nc3nc4c(c(N5CCC[C@H]5CO)n3)CCN(Cc3cccc(OC(F)(F)F)c3)C4)c2)cc(OC)c1OC ; COc1cc(-n2cnc(Nc3nc4c(c(N5CCC[C@H]5CO)n3)CCN(CC(N)=O)C4)c2)cc(OC)c1OC ; COc1cc(-n2cnc(Nc3nc4c(c(N5CCC[C@H]5CO)n3)CCN(Cc3ccc(C#N)cc3)C4)c2)cc(OC)c1OC ; COc1cc(-n2cnc(Nc3nc4c(c(N5CCC[C@H]5CO)n3)CCN(Cc3cccc(C#N)c3)C4)c2)cc(OC)c1OC ; COc1cc2nc(Nc3cn(-c4cc(OC)c(OC)c(OC)c4)cn3)nc(N3CCC[C@H]3CO)c2cc1OC ; COc1cc(-n2cnc(Nc3nc(N4CCC[C@@H]4CO)c4cccn4n3)c2)cc(OC)c1OC] 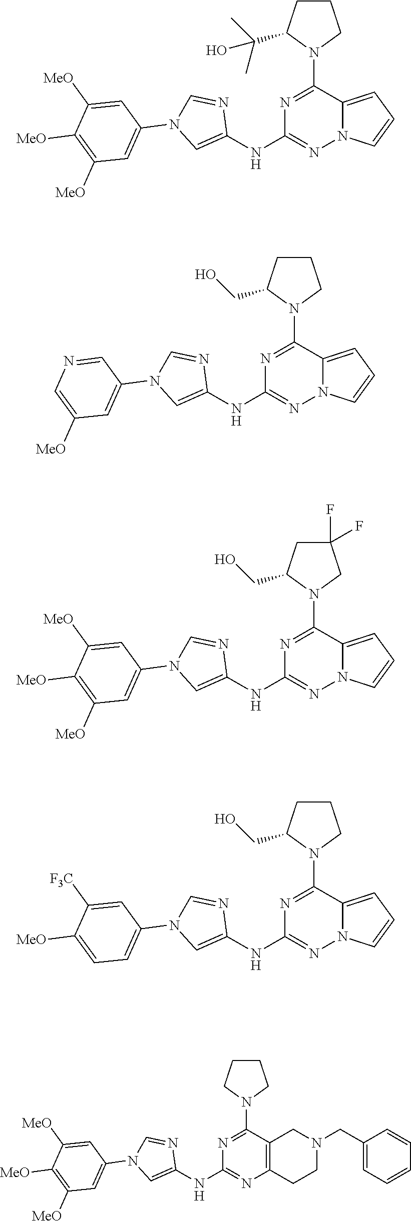 [SMILES: COc1cc(-n2cnc(Nc3nc(N4CCC[C@H]4C(C)(C)O)c4cccn4n3)c2)cc(OC)c1OC ; COc1cncc(-n2cnc(Nc3nc(N4CCC[C@H]4CO)c4cccn4n3)c2)c1 ; COc1cc(-n2cnc(Nc3nc(N4CC(F)(F)C[C@H]4CO)c4cccn4n3)c2)cc(OC)c1OC ; COc1ccc(-n2cnc(Nc3nc(N4CCC[C@H]4CO)c4cccn4n3)c2)cc1C(F)(F)F ; COc1cc(-n2cnc(Nc3nc4c(c(N5CCCC5)n3)CN(Cc3ccccc3)CC4)c2)cc(OC)c1OC] 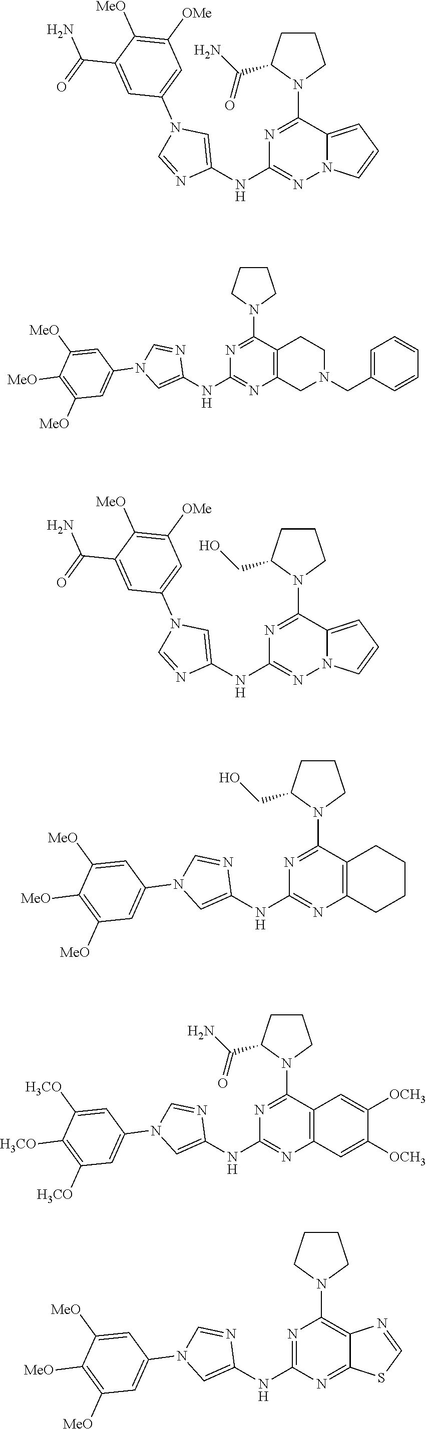 [SMILES: COc1cc(-n2cnc(Nc3nc(N4CCC[C@H]4C(N)=O)c4cccn4n3)c2)cc(C(N)=O)c1OC ; COc1cc(-n2cnc(Nc3nc4c(c(N5CCCC5)n3)CCN(Cc3ccccc3)C4)c2)cc(OC)c1OC ; COc1cc(-n2cnc(Nc3nc(N4CCC[C@H]4CO)c4cccn4n3)c2)cc(C(N)=O)c1OC ; COc1cc(-n2cnc(Nc3nc4c(c(N5CCC[C@H]5CO)n3)CCCC4)c2)cc(OC)c1OC ; COc1cc2nc(Nc3cn(-c4cc(OC)c(OC)c(OC)c4)cn3)nc(N3CCC[C@H]3C(N)=O)c2cc1OC ; COc1cc(-n2cnc(Nc3nc(N4CCCC4)c4ncsc4n3)c2)cc(OC)c1OC] 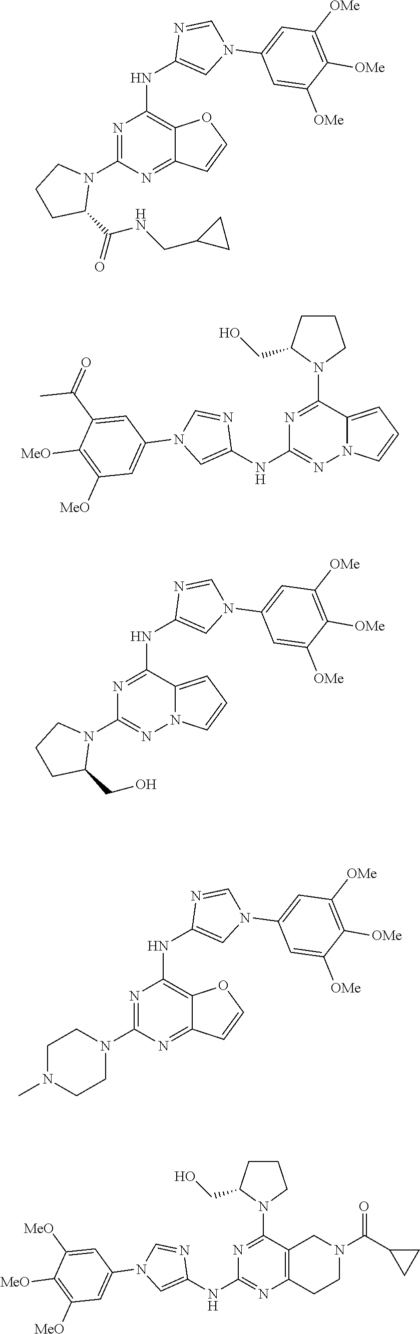 [SMILES: COc1cc(-n2cnc(Nc3nc(N4CCC[C@H]4C(=O)NCC4CC4)nc4ccoc34)c2)cc(OC)c1OC ; COc1cc(-n2cnc(Nc3nc(N4CCC[C@H]4CO)c4cccn4n3)c2)cc(C(C)=O)c1OC ; COc1cc(-n2cnc(Nc3nc(N4CCC[C@@H]4CO)nn4cccc34)c2)cc(OC)c1OC ; COc1cc(-n2cnc(Nc3nc(N4CCN(C)CC4)nc4ccoc34)c2)cc(OC)c1OC ; COc1cc(-n2cnc(Nc3nc4c(c(N5CCC[C@H]5CO)n3)CN(C(=O)C3CC3)CC4)c2)cc(OC)c1OC] 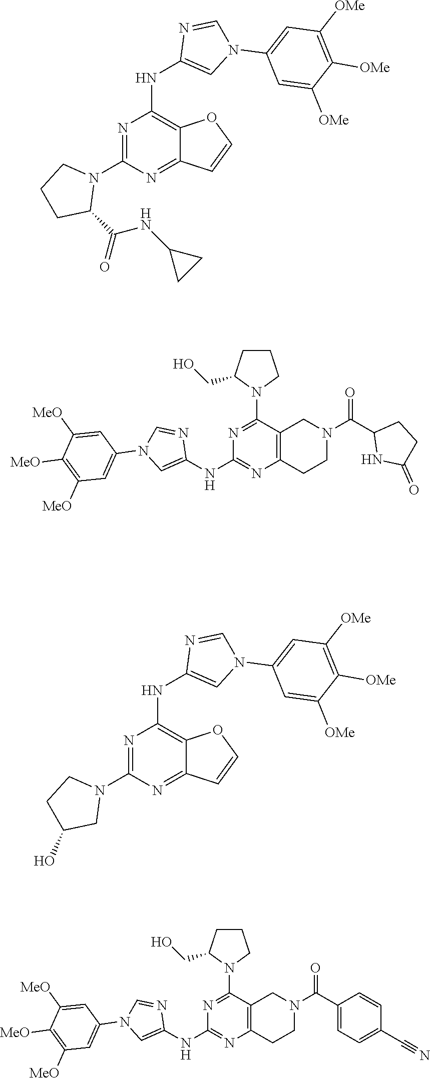 [SMILES: COc1cc(-n2cnc(Nc3nc(N4CCC[C@H]4C(=O)NC4CC4)nc4ccoc34)c2)cc(OC)c1OC ; COc1cc(-n2cnc(Nc3nc4c(c(N5CCC[C@H]5CO)n3)CN(C(=O)C3CCC(=O)N3)CC4)c2)cc(OC)c1OC ; COc1cc(-n2cnc(Nc3nc(N4CC[C@@H](O)C4)nc4ccoc34)c2)cc(OC)c1OC ; COc1cc(-n2cnc(Nc3nc4c(c(N5CCC[C@H]5CO)n3)CN(C(=O)c3ccc(C#N)cc3)CC4)c2)cc(OC)c1OC] 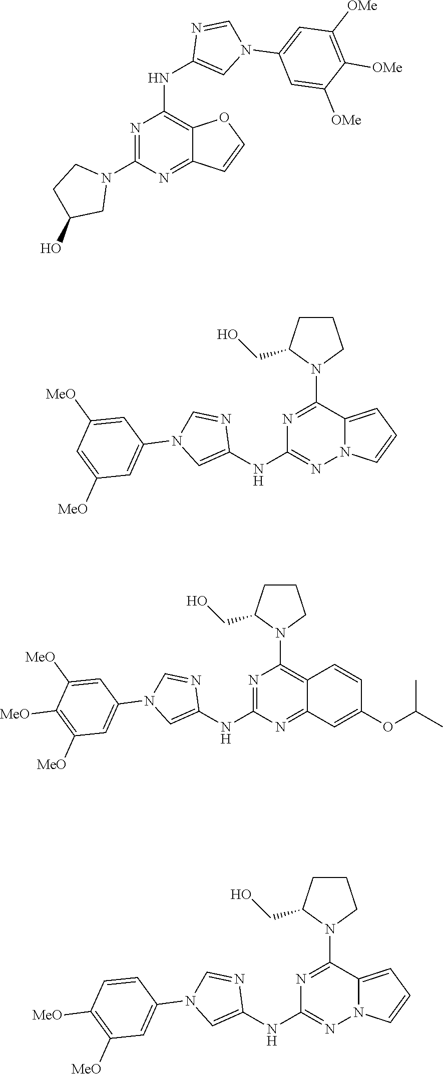 [SMILES: COc1cc(-n2cnc(Nc3nc(N4CC[C@H](O)C4)nc4ccoc34)c2)cc(OC)c1OC ; COc1cc(OC)cc(-n2cnc(Nc3nc(N4CCC[C@H]4CO)c4cccn4n3)c2)c1 ; COc1cc(-n2cnc(Nc3nc(N4CCC[C@H]4CO)c4ccc(OC(C)C)cc4n3)c2)cc(OC)c1OC ; COc1ccc(-n2cnc(Nc3nc(N4CCC[C@H]4CO)c4cccn4n3)c2)cc1OC] 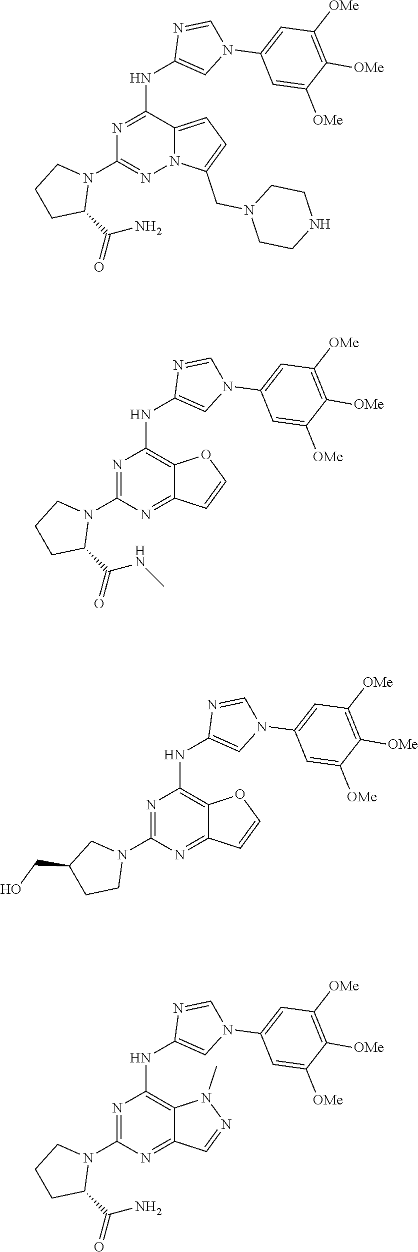 [SMILES: COc1cc(-n2cnc(Nc3nc(N4CCC[C@H]4C(N)=O)nn4c(CN5CCNCC5)ccc34)c2)cc(OC)c1OC ; CNC(=O)[C@@H]1CCCN1c1nc(Nc2cn(-c3cc(OC)c(OC)c(OC)c3)cn2)c2occc2n1 ; COc1cc(-n2cnc(Nc3nc(N4CC[C@@H](CO)C4)nc4ccoc34)c2)cc(OC)c1OC ; COc1cc(-n2cnc(Nc3nc(N4CCC[C@H]4C(N)=O)nc4cnn(C)c34)c2)cc(OC)c1OC] 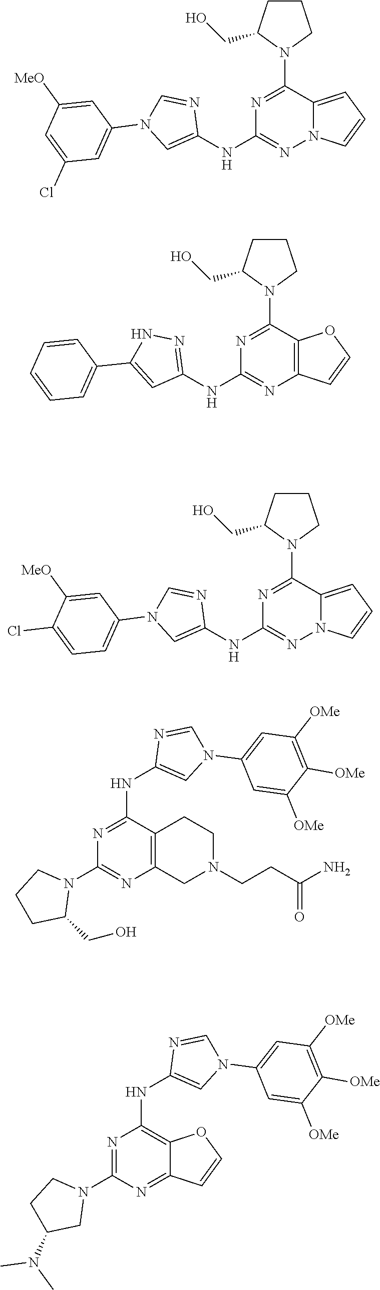 [SMILES: COc1cc(Cl)cc(-n2cnc(Nc3nc(N4CCC[C@H]4CO)c4cccn4n3)c2)c1 ; OC[C@@H]1CCCN1c1nc(Nc2cc(-c3ccccc3)[nH]n2)nc2ccoc12 ; COc1cc(-n2cnc(Nc3nc(N4CCC[C@H]4CO)c4cccn4n3)c2)ccc1Cl ; COc1cc(-n2cnc(Nc3nc(N4CCC[C@H]4CO)nc4c3CCN(CCC(N)=O)C4)c2)cc(OC)c1OC ; COc1cc(-n2cnc(Nc3nc(N4CC[C@@H](N(C)C)C4)nc4ccoc34)c2)cc(OC)c1OC] 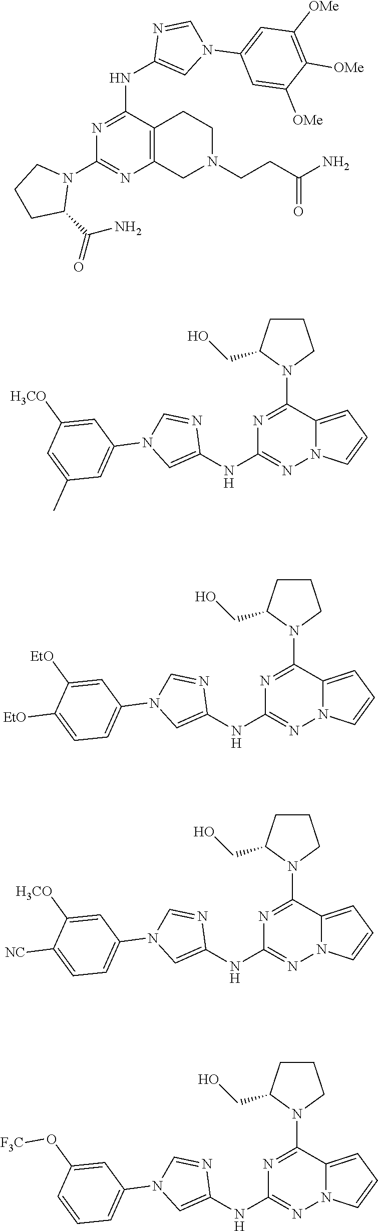 [SMILES: COc1cc(-n2cnc(Nc3nc(N4CCC[C@H]4C(N)=O)nc4c3CCN(CCC(N)=O)C4)c2)cc(OC)c1OC ; COc1cc(C)cc(-n2cnc(Nc3nc(N4CCC[C@H]4CO)c4cccn4n3)c2)c1 ; CCOc1ccc(-n2cnc(Nc3nc(N4CCC[C@H]4CO)c4cccn4n3)c2)cc1OCC ; COc1cc(-n2cnc(Nc3nc(N4CCC[C@H]4CO)c4cccn4n3)c2)ccc1C#N ; OC[C@@H]1CCCN1c1nc(Nc2cn(-c3cccc(OC(F)(F)F)c3)cn2)nn2cccc12] 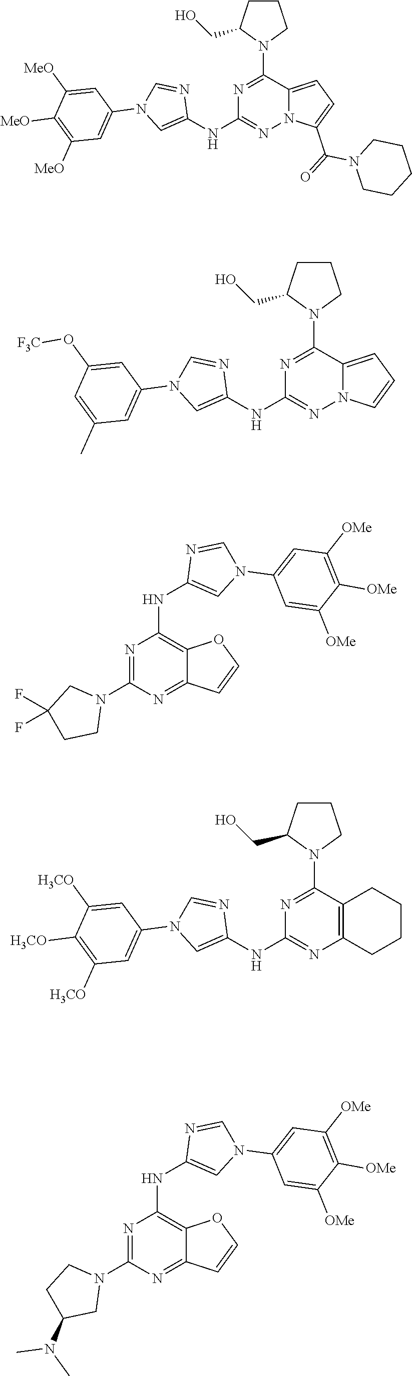 [SMILES: COc1cc(-n2cnc(Nc3nc(N4CCC[C@H]4CO)c4ccc(C(=O)N5CCCCC5)n4n3)c2)cc(OC)c1OC ; Cc1cc(OC(F)(F)F)cc(-n2cnc(Nc3nc(N4CCC[C@H]4CO)c4cccn4n3)c2)c1 ; COc1cc(-n2cnc(Nc3nc(N4CCC(F)(F)C4)nc4ccoc34)c2)cc(OC)c1OC ; COc1cc(-n2cnc(Nc3nc4c(c(N5CCC[C@@H]5CO)n3)CCCC4)c2)cc(OC)c1OC ; COc1cc(-n2cnc(Nc3nc(N4CC[C@H](N(C)C)C4)nc4ccoc34)c2)cc(OC)c1OC] 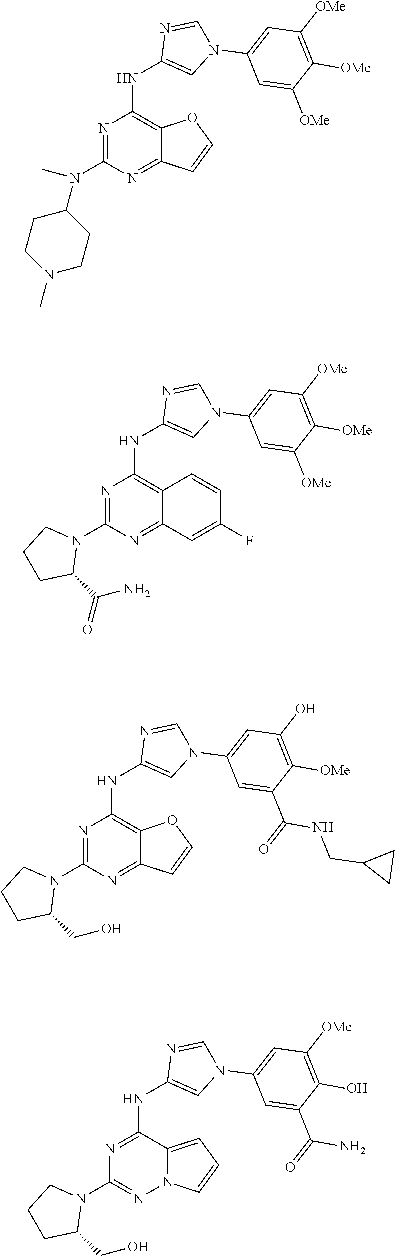 [SMILES: COc1cc(-n2cnc(Nc3nc(N(C)C4CCN(C)CC4)nc4ccoc34)c2)cc(OC)c1OC ; COc1cc(-n2cnc(Nc3nc(N4CCC[C@H]4C(N)=O)nc4cc(F)ccc34)c2)cc(OC)c1OC ; COc1c(O)cc(-n2cnc(Nc3nc(N4CCC[C@H]4CO)nc4ccoc34)c2)cc1C(=O)NCC1CC1 ; COc1cc(-n2cnc(Nc3nc(N4CCC[C@H]4CO)nn4cccc34)c2)cc(C(N)=O)c1O] 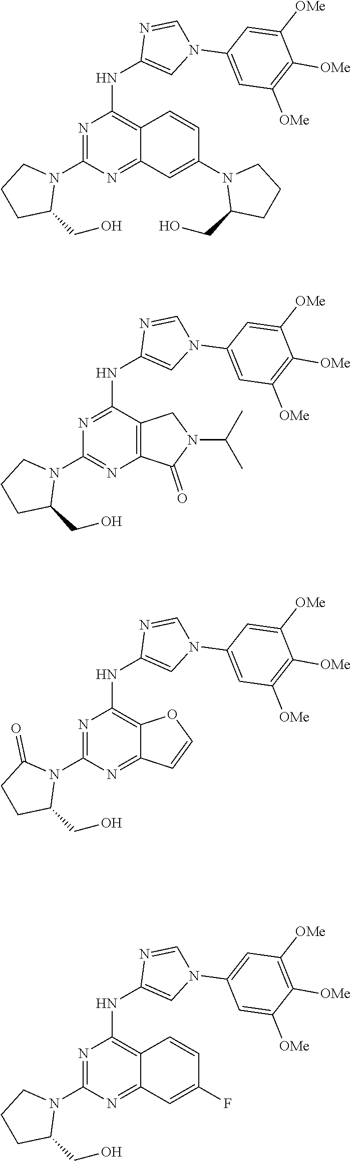 [SMILES: COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4CO)nc4c3CN(C(C)C)C4=O)c2)cc(OC)c1OC ; COc1cc(-n2cnc(Nc3nc(N4C(=O)CC[C@H]4CO)nc4ccoc34)c2)cc(OC)c1OC ; COc1cc(-n2cnc(Nc3nc(N4CCC[C@H]4CO)nc4cc(F)ccc34)c2)cc(OC)c1OC] 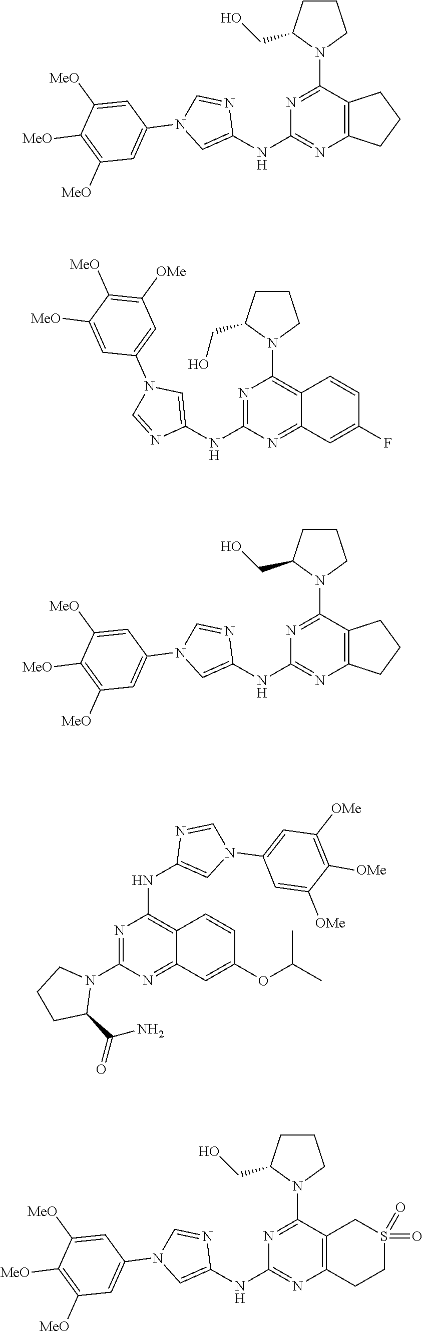 [SMILES: COc1cc(-n2cnc(Nc3nc4c(c(N5CCC[C@H]5CO)n3)CCC4)c2)cc(OC)c1OC ; COc1cc(-n2cnc(Nc3nc(N4CCC[C@H]4CO)c4ccc(F)cc4n3)c2)cc(OC)c1OC ; COc1cc(-n2cnc(Nc3nc4c(c(N5CCC[C@@H]5CO)n3)CCC4)c2)cc(OC)c1OC ; COc1cc(-n2cnc(Nc3nc(N4CCC[C@@H]4C(N)=O)nc4cc(OC(C)C)ccc34)c2)cc(OC)c1OC ; COc1cc(-n2cnc(Nc3nc4c(c(N5CCC[C@H]5CO)n3)CS(=O)(=O)CC4)c2)cc(OC)c1OC] 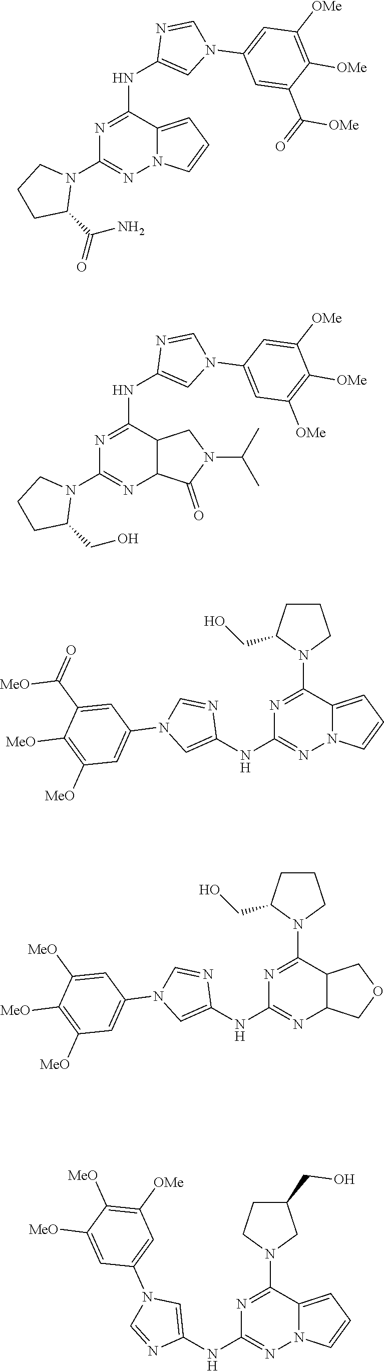 [SMILES: COC(=O)c1cc(-n2cnc(Nc3nc(N4CCC[C@H]4C(N)=O)nn4cccc34)c2)cc(OC)c1OC ; COc1cc(-n2cnc(NC3=NC(N4CCC[C@H]4CO)=NC4C(=O)N(C(C)C)CC34)c2)cc(OC)c1OC ; COC(=O)c1cc(-n2cnc(Nc3nc(N4CCC[C@H]4CO)c4cccn4n3)c2)cc(OC)c1OC ; COc1cc(-n2cnc(NC3=NC4COCC4C(N4CCC[C@H]4CO)=N3)c2)cc(OC)c1OC ; COc1cc(-n2cnc(Nc3nc(N4CC[C@@H](CO)C4)c4cccn4n3)c2)cc(OC)c1OC] 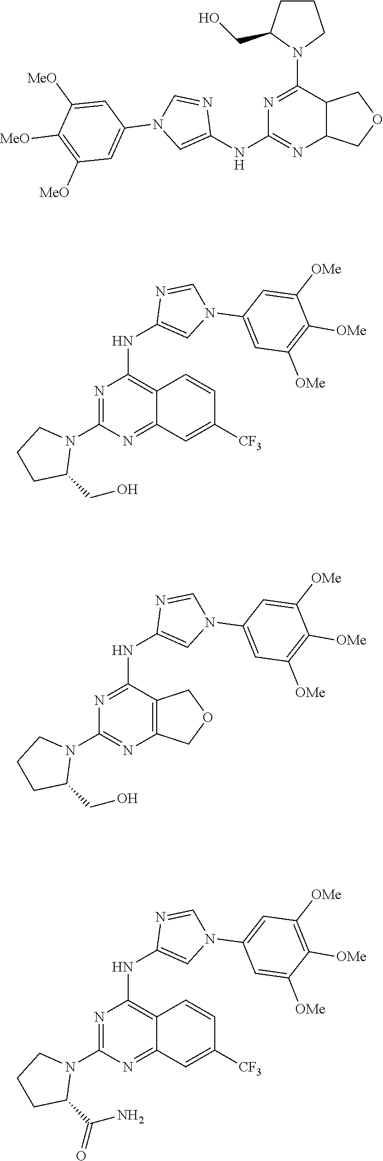 [SMILES: COc1cc(-n2cnc(NC3=NC4COCC4C(N4CCC[C@@H]4CO)=N3)c2)cc(OC)c1OC ; COc1cc(-n2cnc(Nc3nc(N4CCC[C@H]4CO)nc4cc(C(F)(F)F)ccc34)c2)cc(OC)c1OC ; COc1cc(-n2cnc(Nc3nc(N4CCC[C@H]4CO)nc4c3COC4)c2)cc(OC)c1OC ; COc1cc(-n2cnc(Nc3nc(N4CCC[C@H]4C(N)=O)nc4cc(C(F)(F)F)ccc34)c2)cc(OC)c1OC] 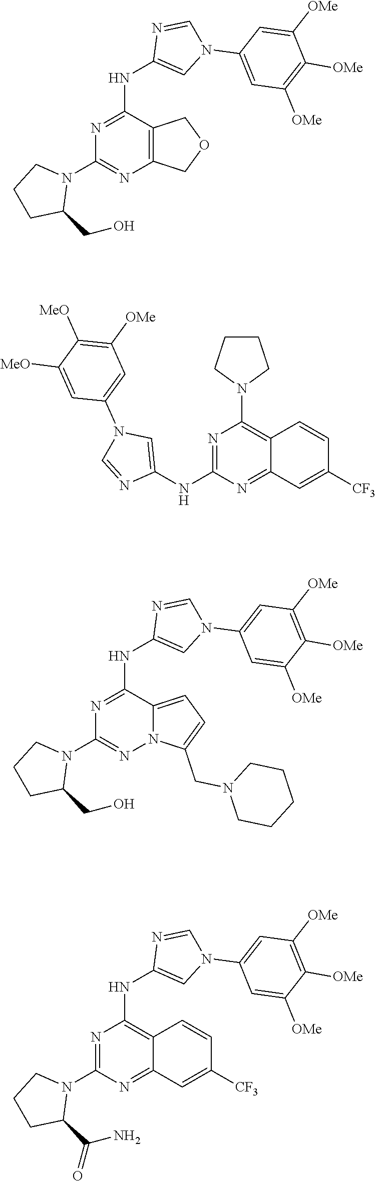 [SMILES: COc1cc(-n2cnc(Nc3nc(N4CCC[C@@H]4CO)nc4c3COC4)c2)cc(OC)c1OC ; COc1cc(-n2cnc(Nc3nc(N4CCCC4)c4ccc(C(F)(F)F)cc4n3)c2)cc(OC)c1OC ; COc1cc(-n2cnc(Nc3nc(N4CCC[C@@H]4CO)nn4c(CN5CCCCC5)ccc34)c2)cc(OC)c1OC ; COc1cc(-n2cnc(Nc3nc(N4CCC[C@@H]4C(N)=O)nc4cc(C(F)(F)F)ccc34)c2)cc(OC)c1OC] 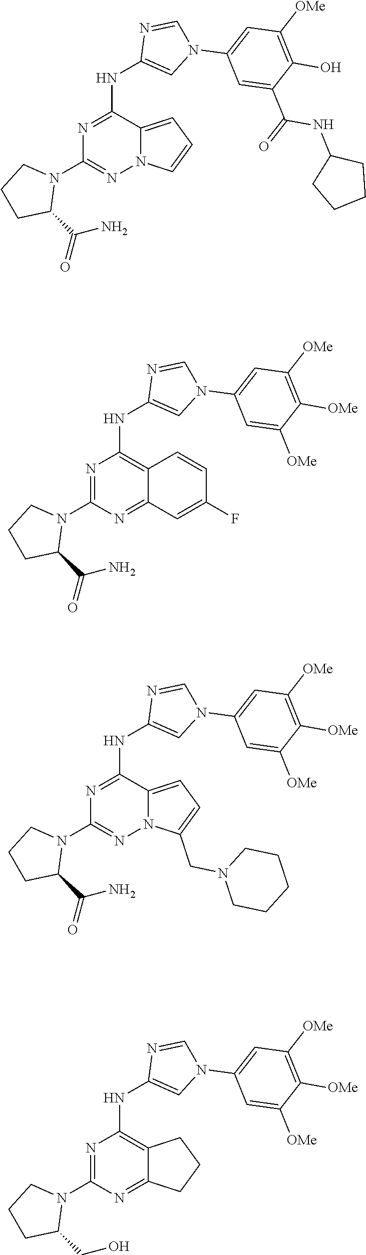 [SMILES: COc1cc(-n2cnc(Nc3nc(N4CCC[C@H]4C(N)=O)nn4cccc34)c2)cc(C(=O)NC2CCCC2)c1O ; COc1cc(-n2cnc(Nc3nc(N4CCC[C@@H]4C(N)=O)nc4cc(F)ccc34)c2)cc(OC)c1OC ; COc1cc(-n2cnc(Nc3nc(N4CCC[C@@H]4C(N)=O)nn4c(CN5CCCCC5)ccc34)c2)cc(OC)c1OC ; COc1cc(-n2cnc(Nc3nc(N4CCC[C@H]4CO)nc4c3CCC4)c2)cc(OC)c1OC] 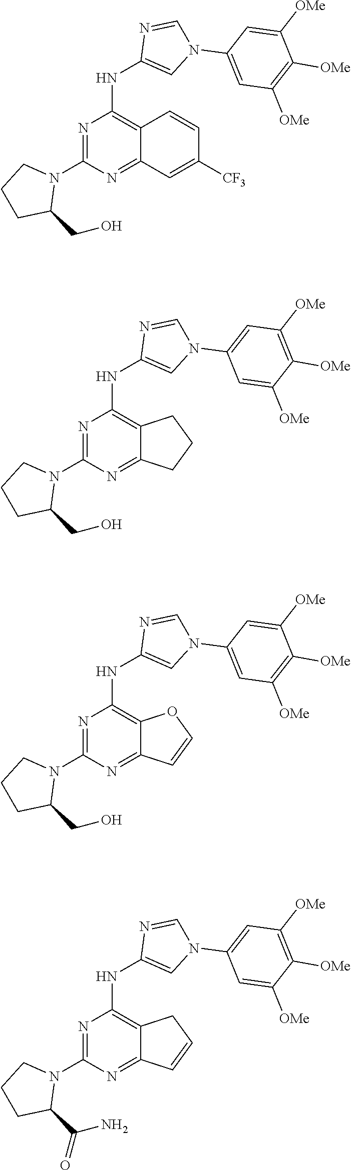 [SMILES: COc1cc(-n2cnc(Nc3nc(N4CCC[C@@H]4CO)nc4cc(C(F)(F)F)ccc34)c2)cc(OC)c1OC ; COc1cc(-n2cnc(Nc3nc(N4CCC[C@@H]4CO)nc4c3CCC4)c2)cc(OC)c1OC ; COc1cc(-n2cnc(Nc3nc(N4CCC[C@@H]4CO)nc4ccoc34)c2)cc(OC)c1OC ; COc1cc(-n2cnc(Nc3nc(N4CCC[C@@H]4C(N)=O)nc4c3CC=C4)c2)cc(OC)c1OC] 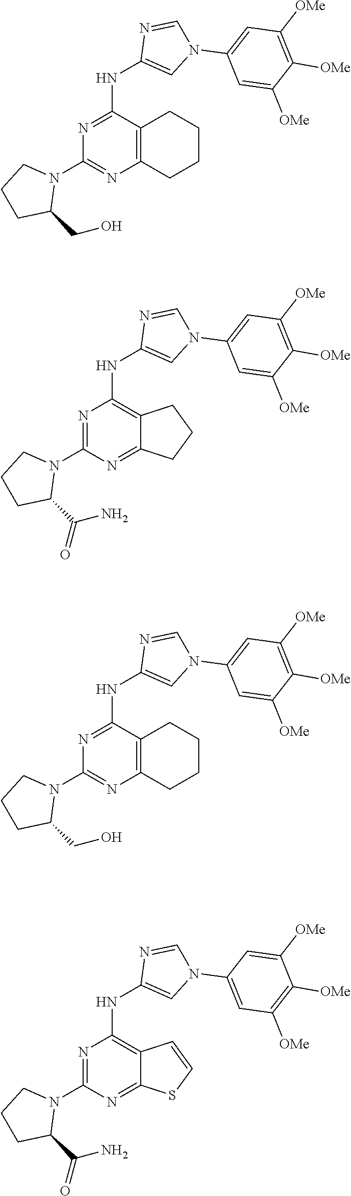 [SMILES: COc1cc(-n2cnc(Nc3nc(N4CCC[C@@H]4CO)nc4c3CCCC4)c2)cc(OC)c1OC ; COc1cc(-n2cnc(Nc3nc(N4CCC[C@H]4C(N)=O)nc4c3CCC4)c2)cc(OC)c1OC ; COc1cc(-n2cnc(Nc3nc(N4CCC[C@H]4CO)nc4c3CCCC4)c2)cc(OC)c1OC ; COc1cc(-n2cnc(Nc3nc(N4CCC[C@@H]4C(N)=O)nc4sccc34)c2)cc(OC)c1OC] 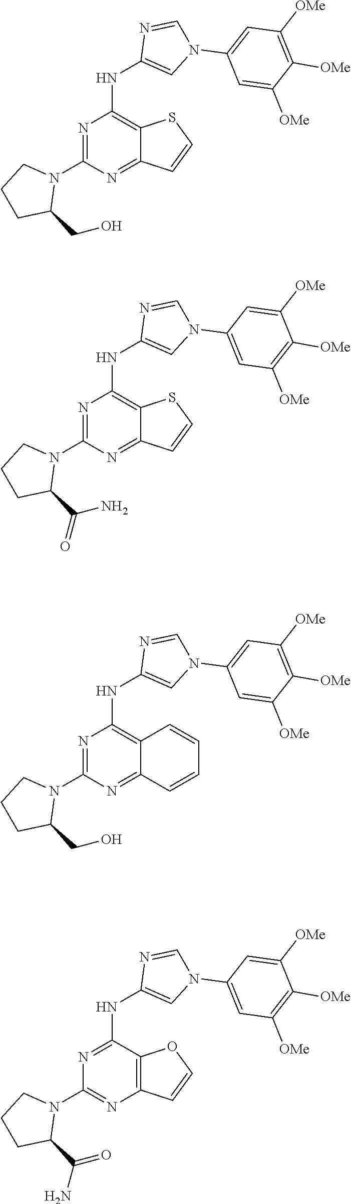 [SMILES: COc1cc(-n2cnc(Nc3nc(N4CCC[C@@H]4CO)nc4ccsc34)c2)cc(OC)c1OC ; COc1cc(-n2cnc(Nc3nc(N4CCC[C@@H]4C(N)=O)nc4ccsc34)c2)cc(OC)c1OC ; COc1cc(-n2cnc(Nc3nc(N4CCC[C@@H]4CO)nc4ccccc34)c2)cc(OC)c1OC ; COc1cc(-n2cnc(Nc3nc(N4CCC[C@@H]4C(N)=O)nc4ccoc34)c2)cc(OC)c1OC] 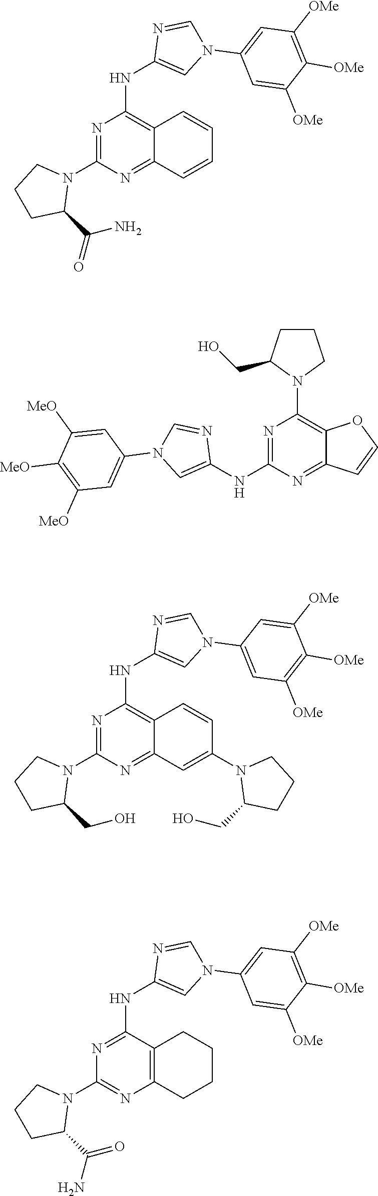 [SMILES: COc1cc(-n2cnc(Nc3nc(N4CCC[C@@H]4C(N)=O)nc4ccccc34)c2)cc(OC)c1OC ; COc1cc(-n2cnc(Nc3nc(N4CCC[C@@H]4CO)c4occc4n3)c2)cc(OC)c1OC ; COc1cc(-n2cnc(Nc3nc(N4CCC[C@@H]4CO)nc4cc(N5CCC[C@@H]5CO)ccc34)c2)cc(OC)c1OC ; COc1cc(-n2cnc(Nc3nc(N4CCC[C@H]4C(N)=O)nc4c3CCCC4)c2)cc(OC)c1OC] 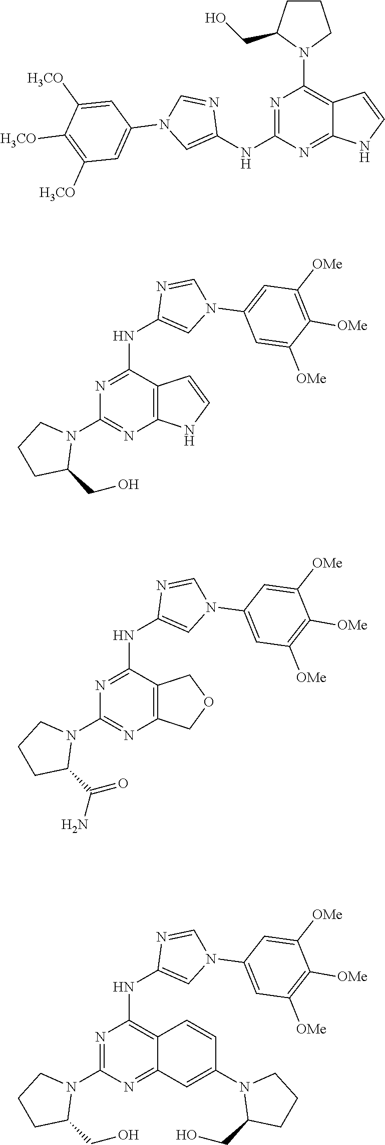 [SMILES: COc1cc(-n2cnc(Nc3nc(N4CCC[C@@H]4CO)c4cc[nH]c4n3)c2)cc(OC)c1OC ; COc1cc(-n2cnc(Nc3nc(N4CCC[C@@H]4CO)nc4[nH]ccc34)c2)cc(OC)c1OC ; COc1cc(-n2cnc(Nc3nc(N4CCC[C@H]4C(N)=O)nc4c3COC4)c2)cc(OC)c1OC ; COc1cc(-n2cnc(Nc3nc(N4CCC[C@H]4CO)nc4cc(N5CCC[C@H]5CO)ccc34)c2)cc(OC)c1OC] 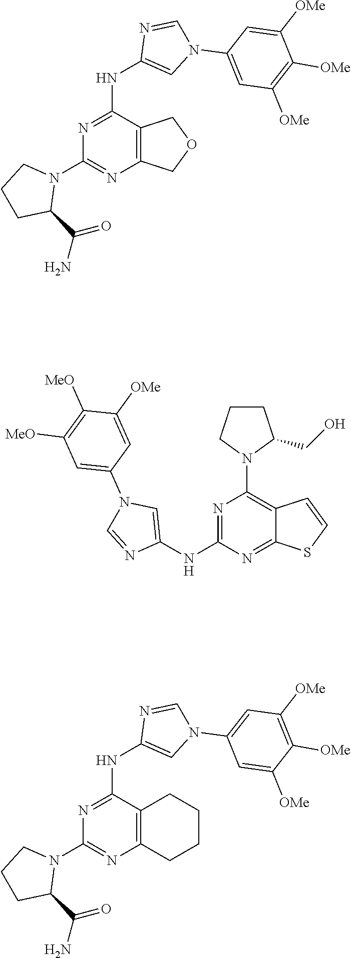 [SMILES: COc1cc(-n2cnc(Nc3nc(N4CCC[C@@H]4C(N)=O)nc4c3COC4)c2)cc(OC)c1OC ; COc1cc(-n2cnc(Nc3nc(N4CCC[C@@H]4CO)c4ccsc4n3)c2)cc(OC)c1OC ; COc1cc(-n2cnc(Nc3nc(N4CCC[C@@H]4C(N)=O)nc4c3CCCC4)c2)cc(OC)c1OC] 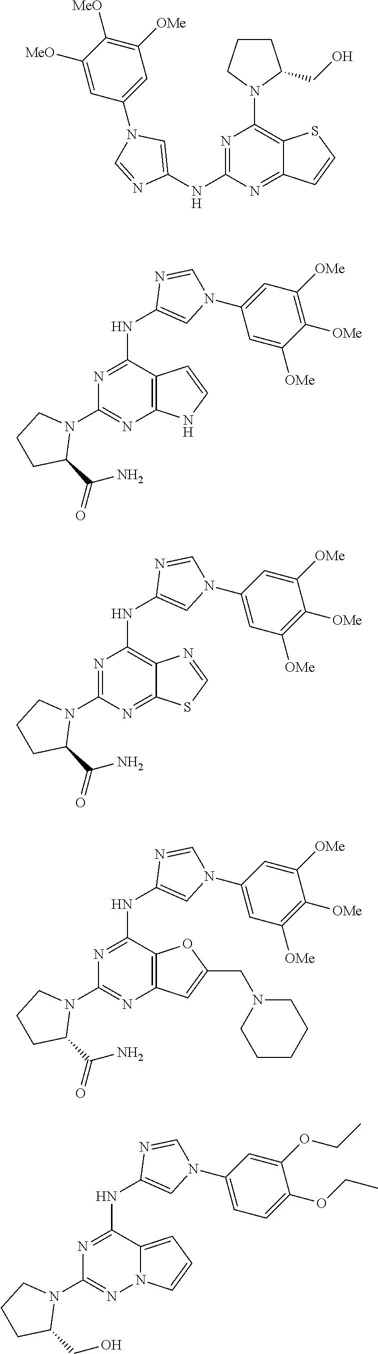 [SMILES: COc1cc(-n2cnc(Nc3nc(N4CCC[C@@H]4CO)c4sccc4n3)c2)cc(OC)c1OC ; COc1cc(-n2cnc(Nc3nc(N4CCC[C@@H]4C(N)=O)nc4[nH]ccc34)c2)cc(OC)c1OC ; COc1cc(-n2cnc(Nc3nc(N4CCC[C@@H]4C(N)=O)nc4scnc34)c2)cc(OC)c1OC ; COc1cc(-n2cnc(Nc3nc(N4CCC[C@H]4C(N)=O)nc4cc(CN5CCCCC5)oc34)c2)cc(OC)c1OC ; CCOc1ccc(-n2cnc(Nc3nc(N4CCC[C@H]4CO)nn4cccc34)c2)cc1OCC] 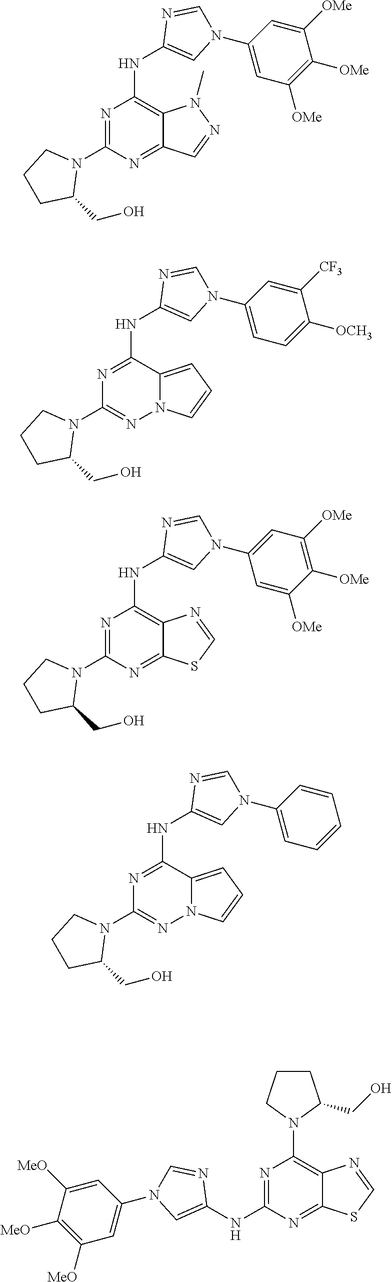 [SMILES: COc1cc(-n2cnc(Nc3nc(N4CCC[C@H]4CO)nc4cnn(C)c34)c2)cc(OC)c1OC ; COc1ccc(-n2cnc(Nc3nc(N4CCC[C@H]4CO)nn4cccc34)c2)cc1C(F)(F)F ; COc1cc(-n2cnc(Nc3nc(N4CCC[C@@H]4CO)nc4scnc34)c2)cc(OC)c1OC ; OC[C@@H]1CCCN1c1nc(Nc2cn(-c3ccccc3)cn2)c2cccn2n1 ; COc1cc(-n2cnc(Nc3nc(N4CCC[C@@H]4CO)c4ncsc4n3)c2)cc(OC)c1OC] 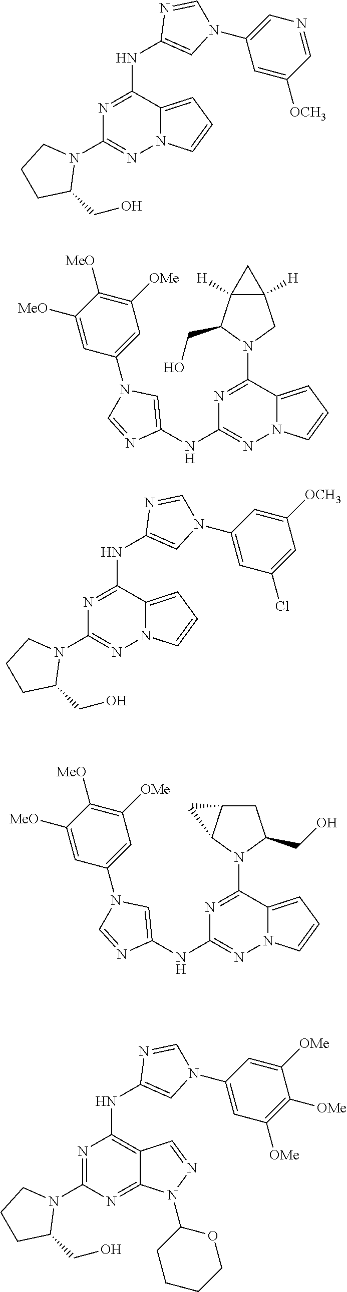 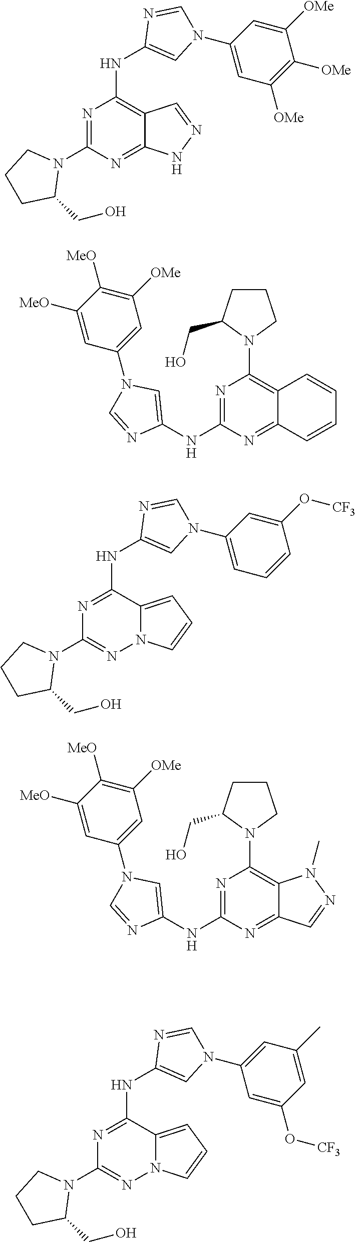 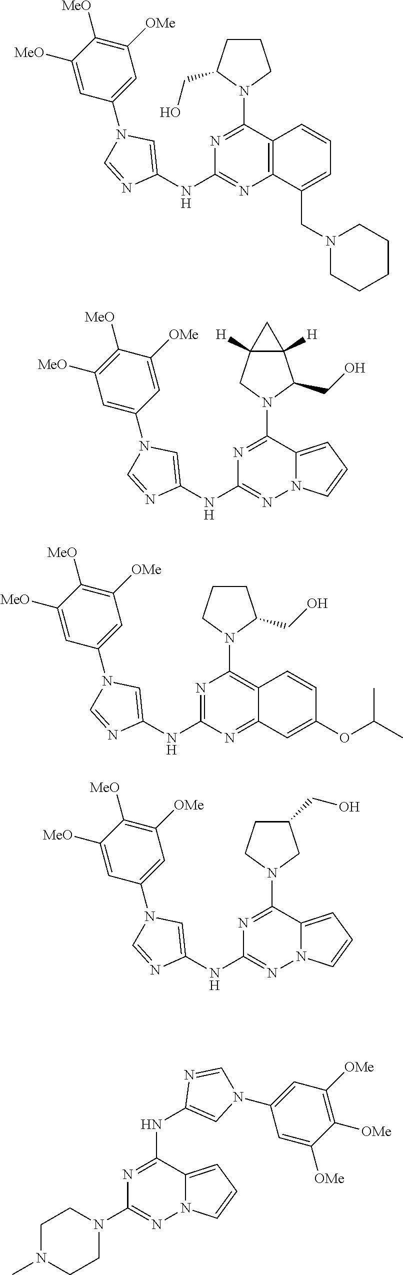 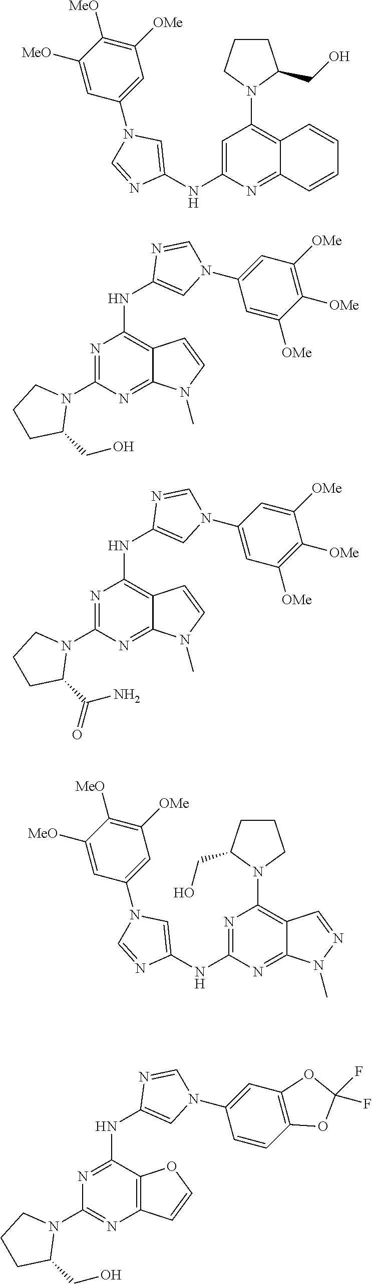 [SMILES: COc1cc(-n2cnc(Nc3cc(N4CCC[C@H]4CO)c4ccccc4n3)c2)cc(OC)c1OC ; COc1cc(-n2cnc(Nc3nc(N4CCC[C@H]4CO)nc4c3ccn4C)c2)cc(OC)c1OC ; COc1cc(-n2cnc(Nc3nc(N4CCC[C@H]4C(N)=O)nc4c3ccn4C)c2)cc(OC)c1OC ; COc1cc(-n2cnc(Nc3nc(N4CCC[C@H]4CO)c4cnn(C)c4n3)c2)cc(OC)c1OC ; OC[C@@H]1CCCN1c1nc(Nc2cn(-c3ccc4c(c3)OC(F)(F)O4)cn2)c2occc2n1] 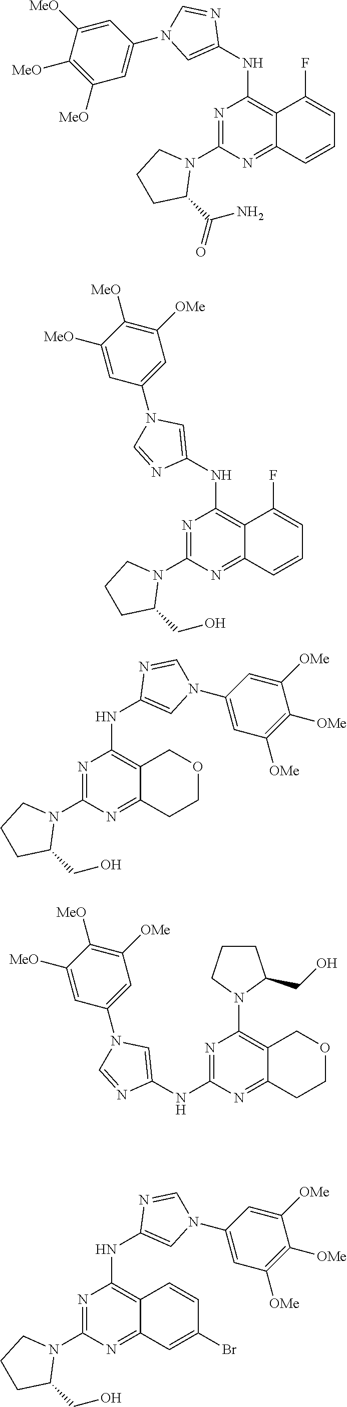 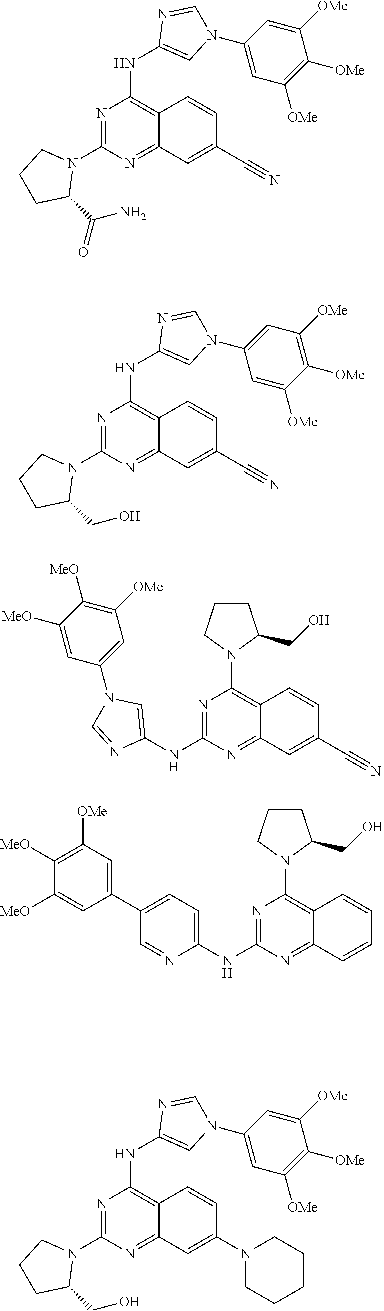 [SMILES: COc1cc(-n2cnc(Nc3nc(N4CCC[C@H]4C(N)=O)nc4cc(C#N)ccc34)c2)cc(OC)c1OC ; COc1cc(-n2cnc(Nc3nc(N4CCC[C@H]4CO)nc4cc(C#N)ccc34)c2)cc(OC)c1OC ; COc1cc(-n2cnc(Nc3nc(N4CCC[C@H]4CO)c4ccc(C#N)cc4n3)c2)cc(OC)c1OC ; COc1cc(-c2ccc(Nc3nc(N4CCC[C@H]4CO)c4ccccc4n3)nc2)cc(OC)c1OC ; COc1cc(-n2cnc(Nc3nc(N4CCC[C@H]4CO)nc4cc(N5CCCCC5)ccc34)c2)cc(OC)c1OC] 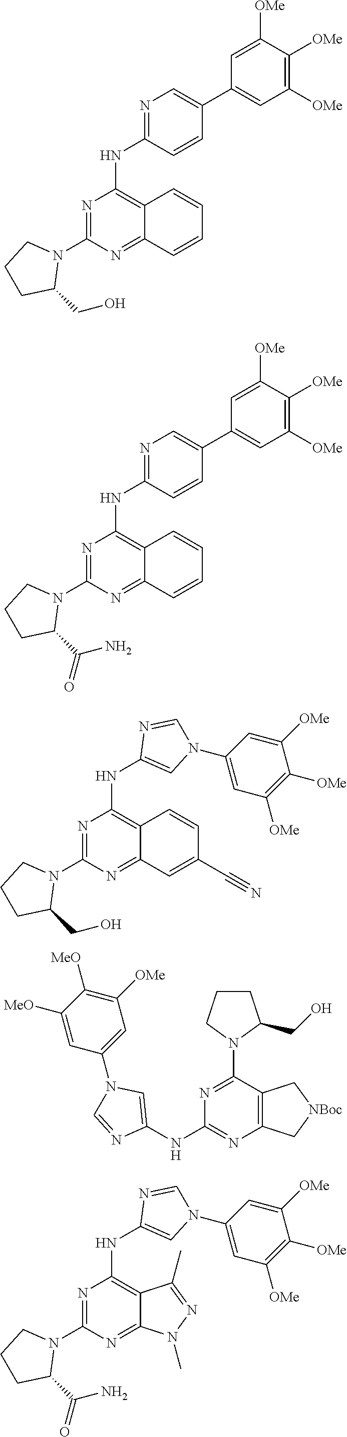 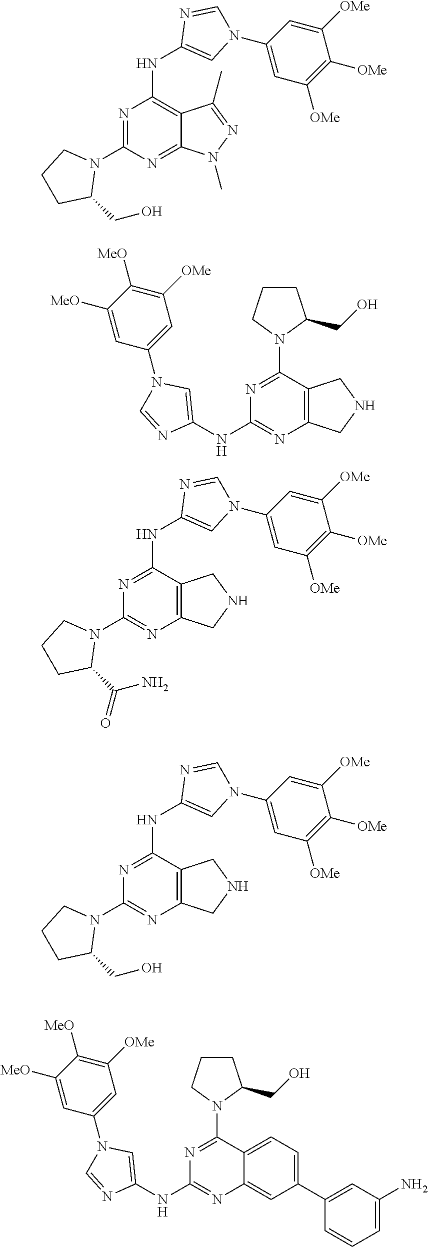 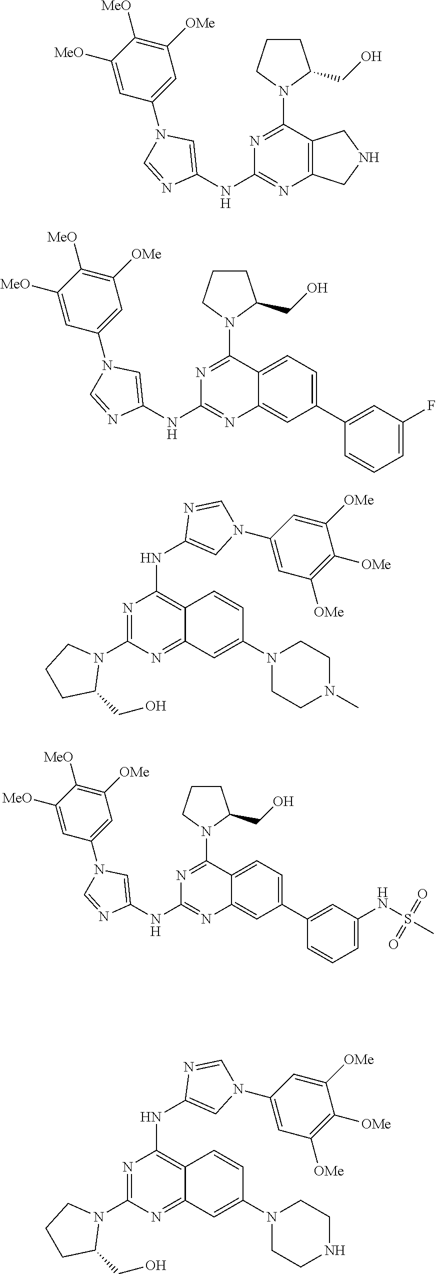 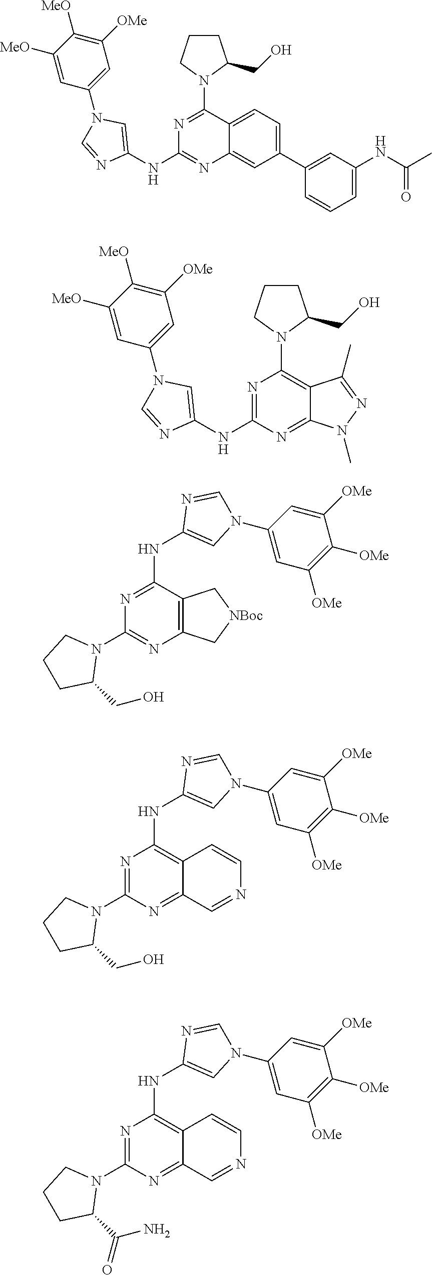 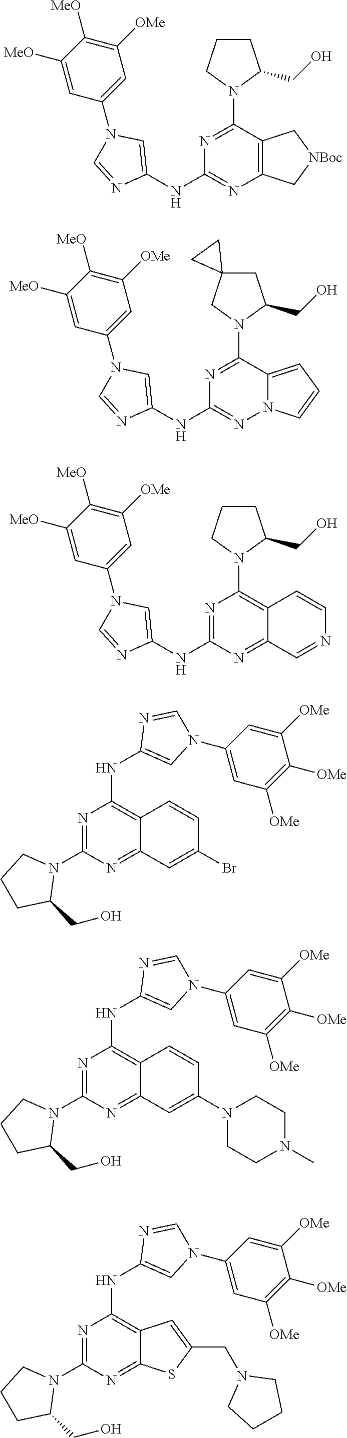 [SMILES: COc1cc(-n2cnc(Nc3nc4c(c(N5CCC[C@@H]5CO)n3)CN(C(=O)OC(C)(C)C)C4)c2)cc(OC)c1OC ; COc1cc(-n2cnc(Nc3nc(N4CC5(CC5)C[C@H]4CO)c4cccn4n3)c2)cc(OC)c1OC ; COc1cc(-n2cnc(Nc3nc(N4CCC[C@H]4CO)c4ccncc4n3)c2)cc(OC)c1OC ; COc1cc(-n2cnc(Nc3nc(N4CCC[C@@H]4CO)nc4cc(Br)ccc34)c2)cc(OC)c1OC ; COc1cc(-n2cnc(Nc3nc(N4CCC[C@@H]4CO)nc4cc(N5CCN(C)CC5)ccc34)c2)cc(OC)c1OC ; COc1cc(-n2cnc(Nc3nc(N4CCC[C@H]4CO)nc4sc(CN5CCCC5)cc34)c2)cc(OC)c1OC] 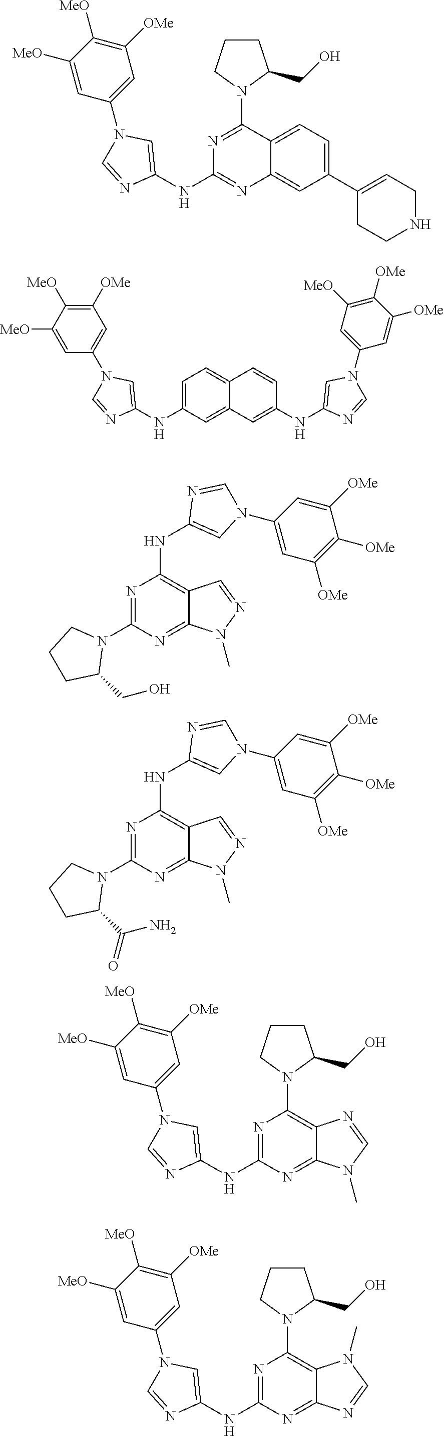 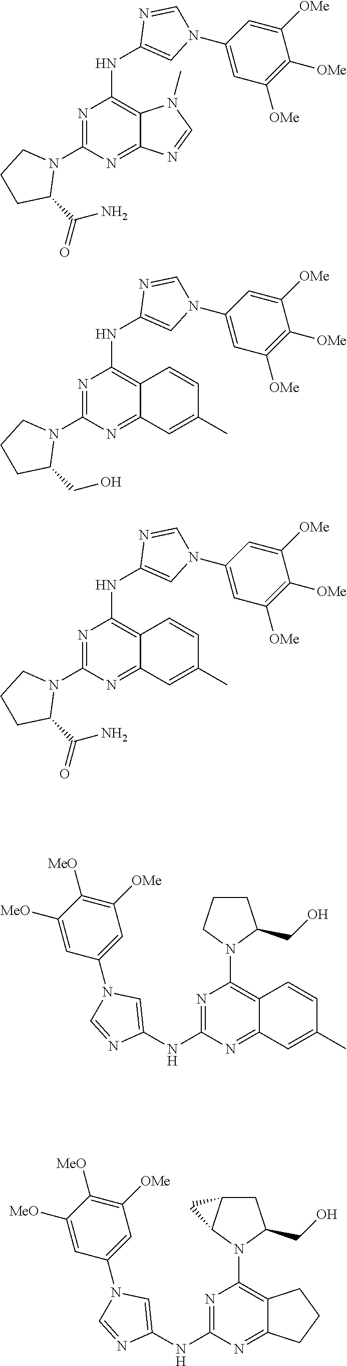 [SMILES: COc1cc(-n2cnc(Nc3nc(N4CCC[C@H]4C(N)=O)nc4ncn(C)c34)c2)cc(OC)c1OC ; COc1cc(-n2cnc(Nc3nc(N4CCC[C@H]4CO)nc4cc(C)ccc34)c2)cc(OC)c1OC ; COc1cc(-n2cnc(Nc3nc(N4CCC[C@H]4C(N)=O)nc4cc(C)ccc34)c2)cc(OC)c1OC ; COc1cc(-n2cnc(Nc3nc(N4CCC[C@H]4CO)c4ccc(C)cc4n3)c2)cc(OC)c1OC ; COc1cc(-n2cnc(Nc3nc4c(c(N5[C@H](CO)C[C@H]6C[C@H]65)n3)CCC4)c2)cc(OC)c1OC] 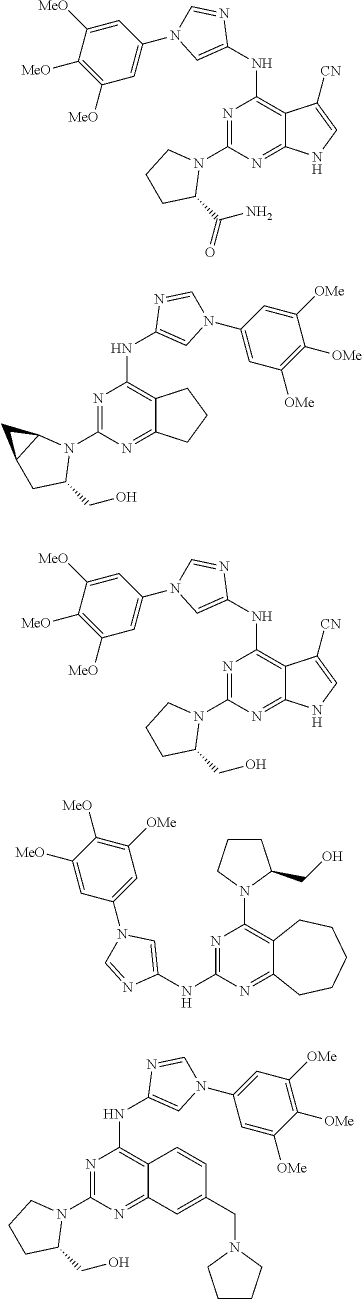 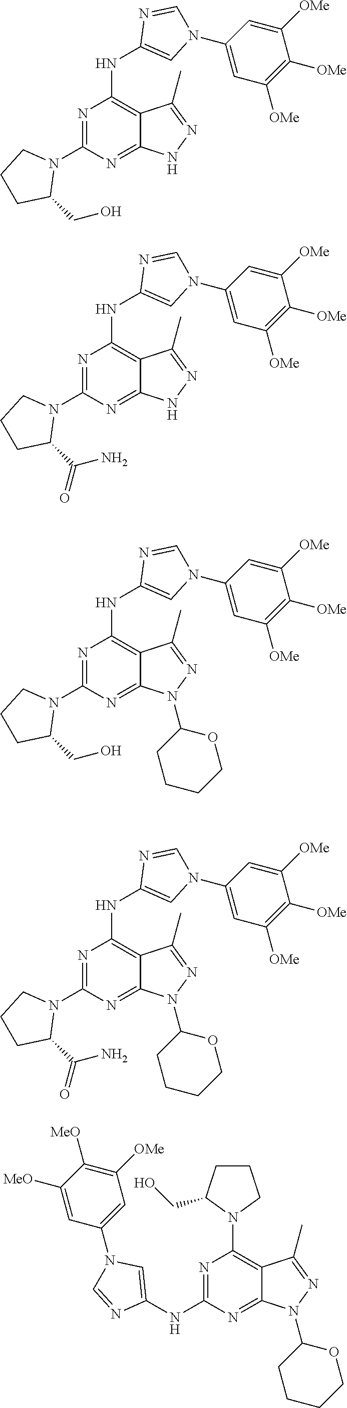 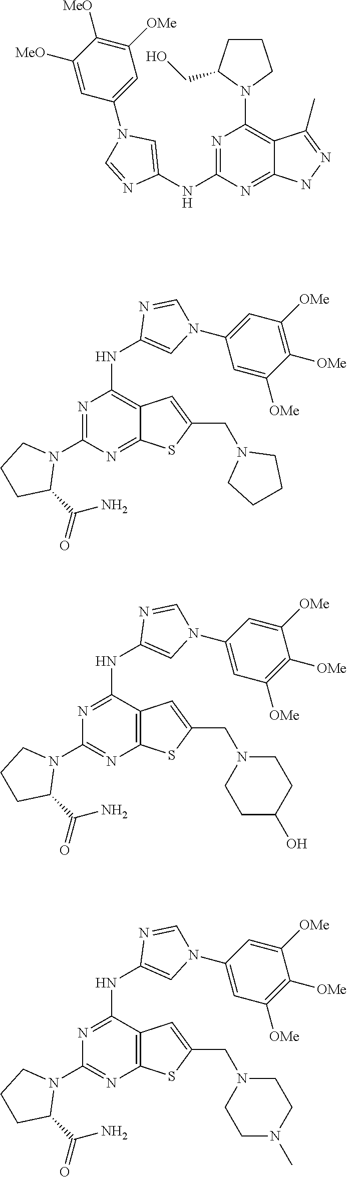 [SMILES: COc1cc(-n2cnc(Nc3nc(N4CCC[C@H]4CO)c4c(C)n[nH]c4n3)c2)cc(OC)c1OC ; COc1cc(-n2cnc(Nc3nc(N4CCC[C@H]4C(N)=O)nc4sc(CN5CCCC5)cc34)c2)cc(OC)c1OC ; COc1cc(-n2cnc(Nc3nc(N4CCC[C@H]4C(N)=O)nc4sc(CN5CCC(O)CC5)cc34)c2)cc(OC)c1OC ; COc1cc(-n2cnc(Nc3nc(N4CCC[C@H]4C(N)=O)nc4sc(CN5CCN(C)CC5)cc34)c2)cc(OC)c1OC] 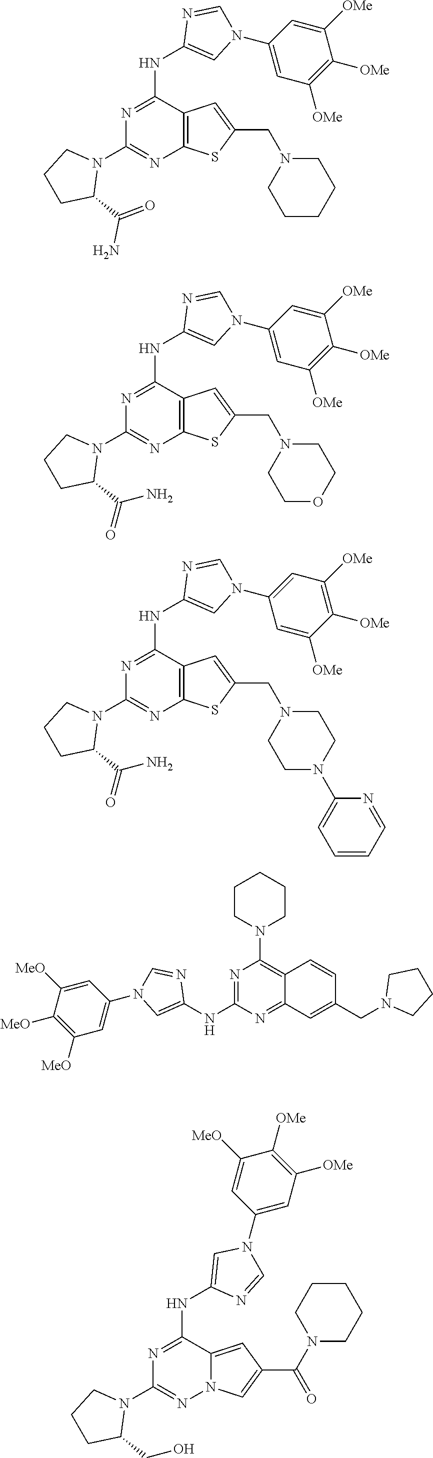 [SMILES: COc1cc(-n2cnc(Nc3nc(N4CCC[C@H]4C(N)=O)nc4sc(CN5CCCCC5)cc34)c2)cc(OC)c1OC ; COc1cc(-n2cnc(Nc3nc(N4CCC[C@H]4C(N)=O)nc4sc(CN5CCOCC5)cc34)c2)cc(OC)c1OC ; COc1cc(-n2cnc(Nc3nc(N4CCC[C@H]4C(N)=O)nc4sc(CN5CCN(c6ccccn6)CC5)cc34)c2)cc(OC)c1OC ; COc1cc(-n2cnc(Nc3nc(N4CCCCC4)c4ccc(CN5CCCC5)cc4n3)c2)cc(OC)c1OC ; COc1cc(-n2cnc(Nc3nc(N4CCC[C@H]4CO)nn4cc(C(=O)N5CCCCC5)cc34)c2)cc(OC)c1OC] 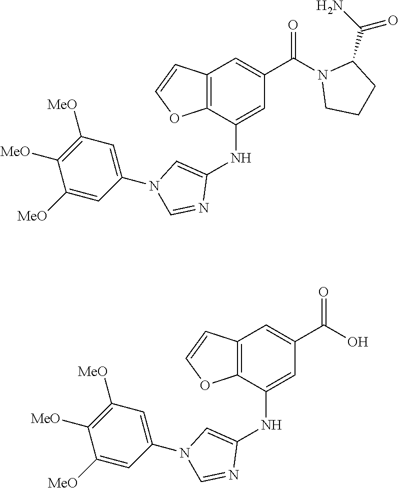 [SMILES: COc1cc(-n2cnc(Nc3cc(C(=O)N4CCC[C@H]4C(N)=O)cc4ccoc34)c2)cc(OC)c1OC ; COc1cc(-n2cnc(Nc3cc(C(=O)O)cc4ccoc34)c2)cc(OC)c1OC] 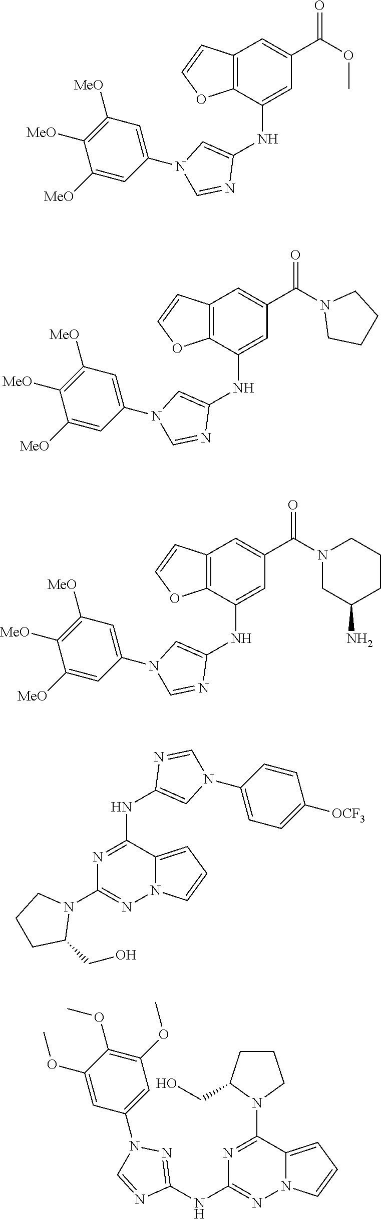 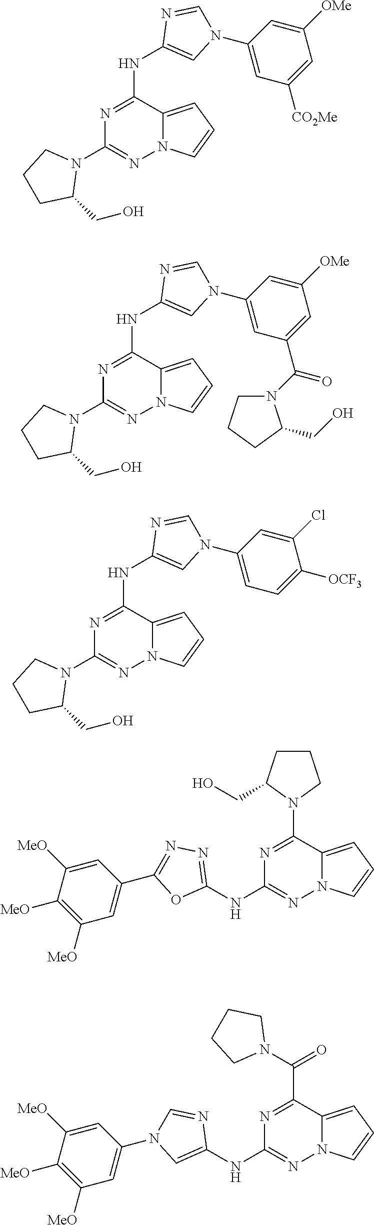 [SMILES: COC(=O)c1cc(OC)cc(-n2cnc(Nc3nc(N4CCC[C@H]4CO)nn4cccc34)c2)c1 ; COc1cc(C(=O)N2CCC[C@H]2CO)cc(-n2cnc(Nc3nc(N4CCC[C@H]4CO)nn4cccc34)c2)c1 ; OC[C@@H]1CCCN1c1nc(Nc2cn(-c3ccc(OC(F)(F)F)c(Cl)c3)cn2)c2cccn2n1 ; COc1cc(-c2nnc(Nc3nc(N4CCC[C@H]4CO)c4cccn4n3)o2)cc(OC)c1OC ; COc1cc(-n2cnc(Nc3nc(C(=O)N4CCCC4)c4cccn4n3)c2)cc(OC)c1OC] 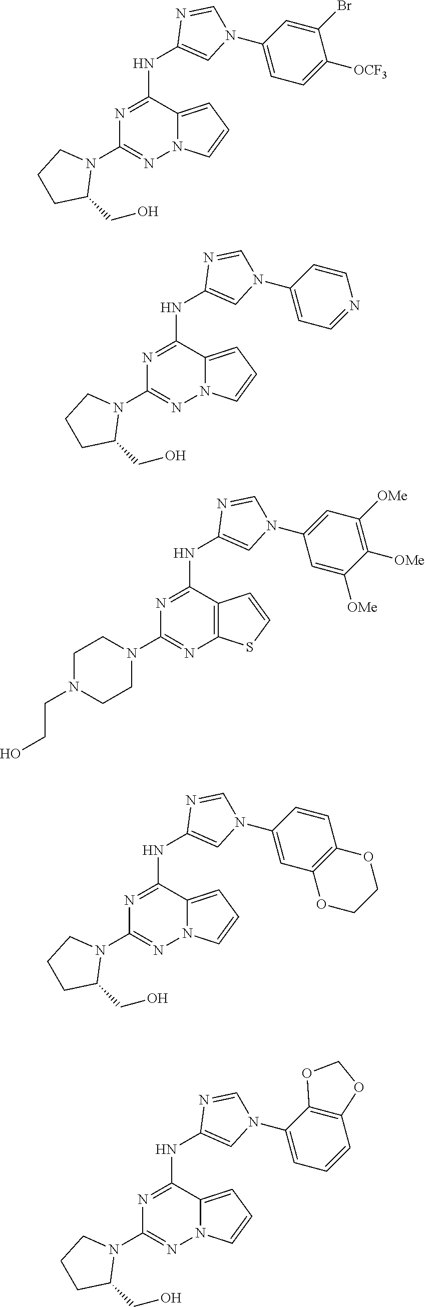 [SMILES: OC[C@@H]1CCCN1c1nc(Nc2cn(-c3ccc(OC(F)(F)F)c(Br)c3)cn2)c2cccn2n1 ; OC[C@@H]1CCCN1c1nc(Nc2cn(-c3ccncc3)cn2)c2cccn2n1 ; COc1cc(-n2cnc(Nc3nc(N4CCN(CCO)CC4)nc4sccc34)c2)cc(OC)c1OC ; OC[C@@H]1CCCN1c1nc(Nc2cn(-c3ccc4c(c3)OCCO4)cn2)c2cccn2n1 ; OC[C@@H]1CCCN1c1nc(Nc2cn(-c3cccc4c3OCO4)cn2)c2cccn2n1] 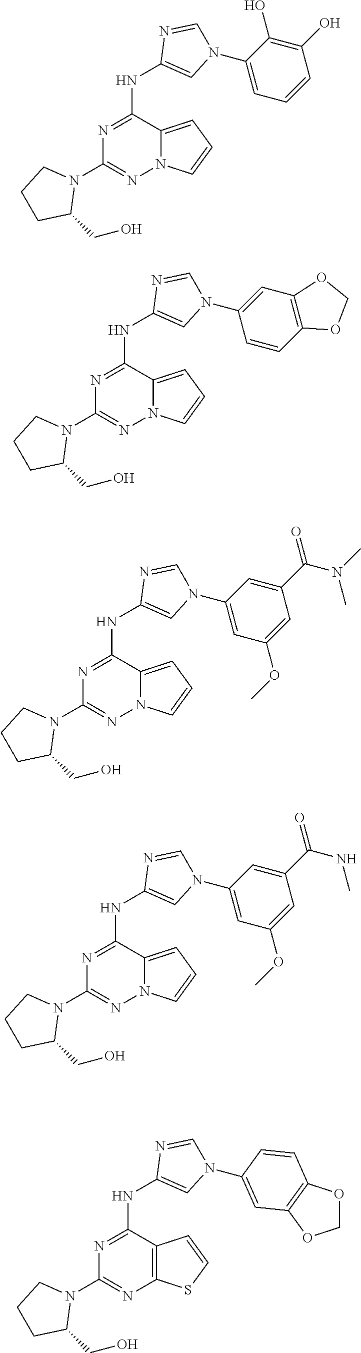 [SMILES: OC[C@@H]1CCCN1c1nc(Nc2cn(-c3cccc(O)c3O)cn2)c2cccn2n1 ; OC[C@@H]1CCCN1c1nc(Nc2cn(-c3ccc4c(c3)OCO4)cn2)c2cccn2n1 ; COc1cc(C(=O)N(C)C)cc(-n2cnc(Nc3nc(N4CCC[C@H]4CO)nn4cccc34)c2)c1 ; CNC(=O)c1cc(OC)cc(-n2cnc(Nc3nc(N4CCC[C@H]4CO)nn4cccc34)c2)c1 ; OC[C@@H]1CCCN1c1nc(Nc2cn(-c3ccc4c(c3)OCO4)cn2)c2ccsc2n1] 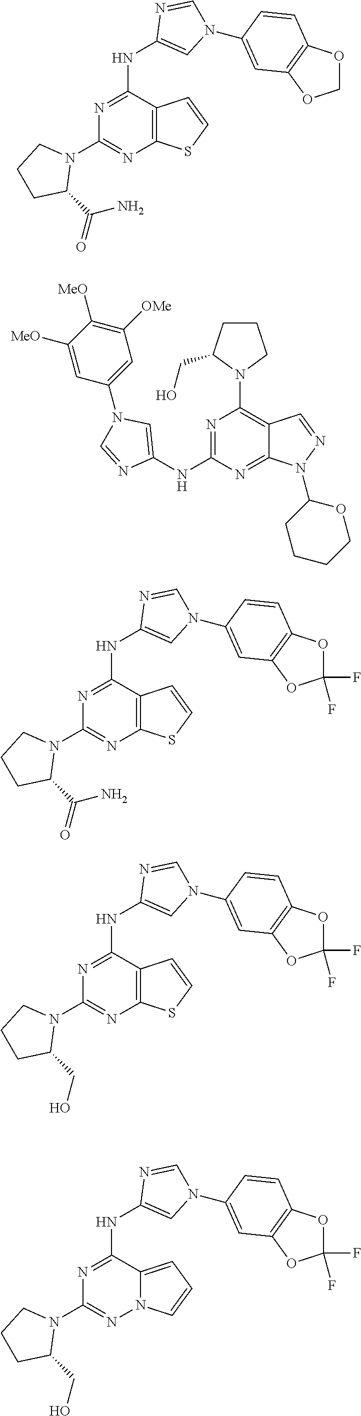 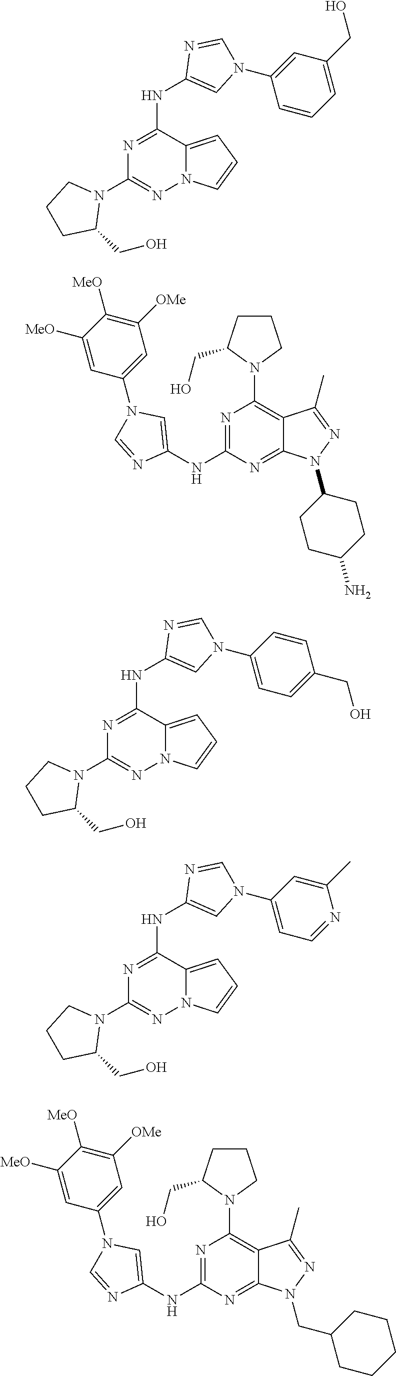 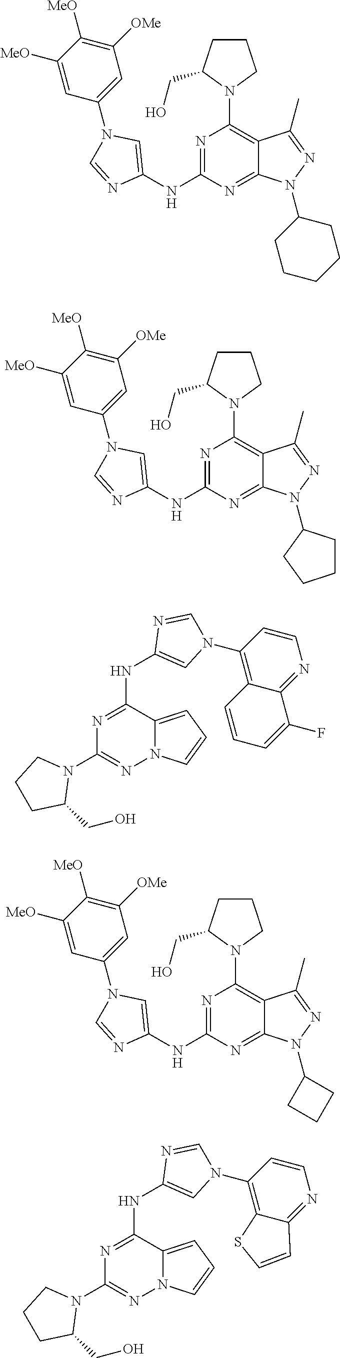 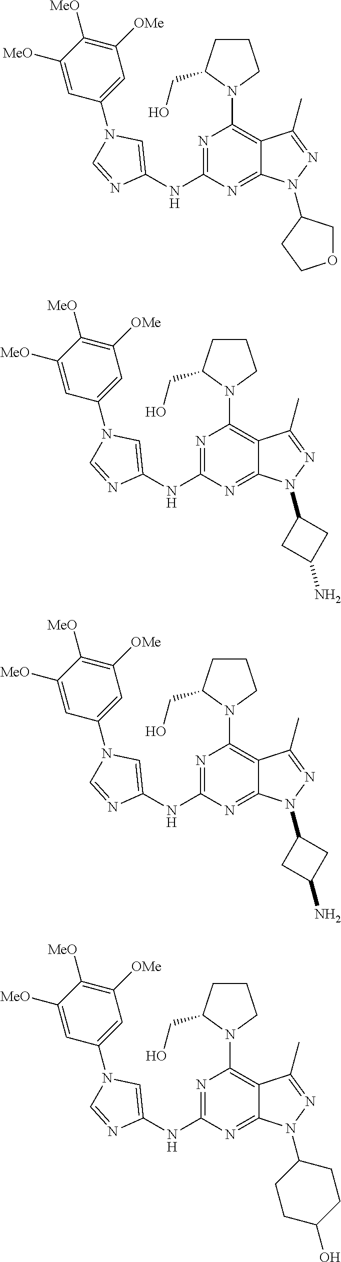 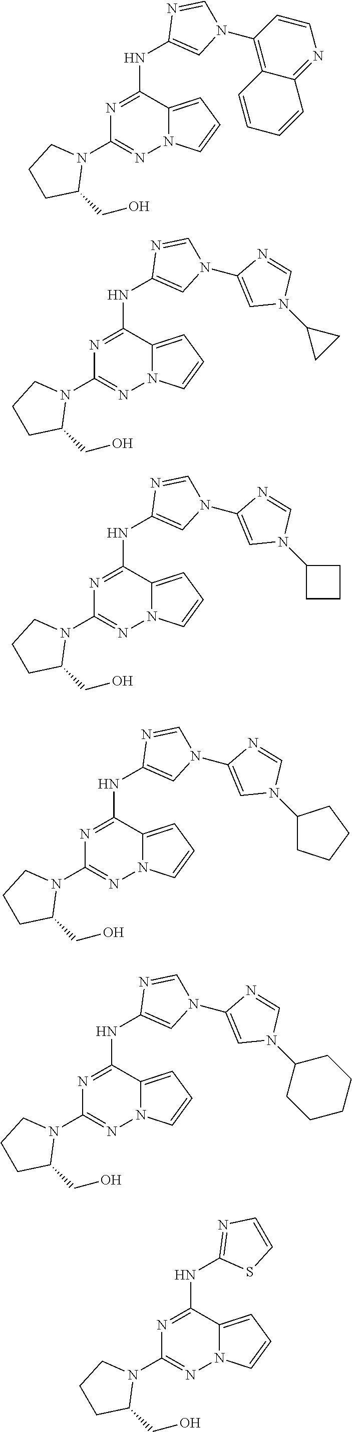 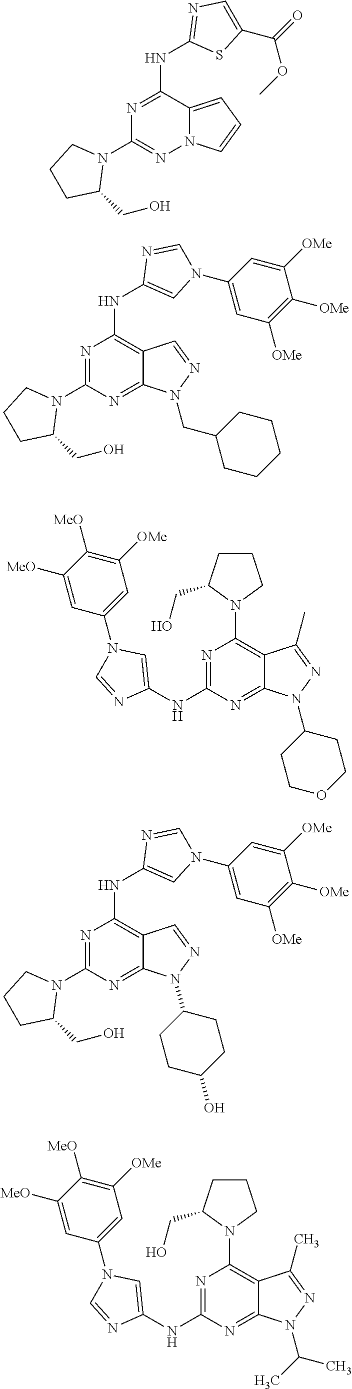 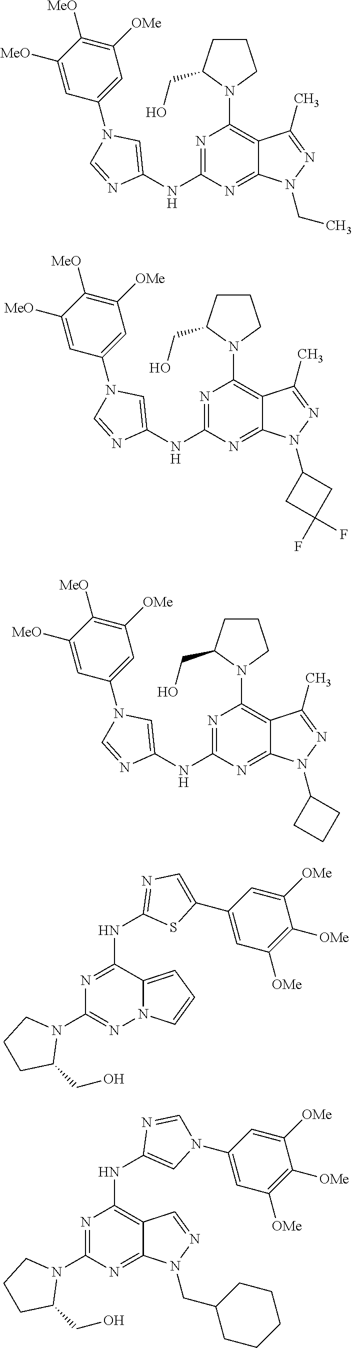 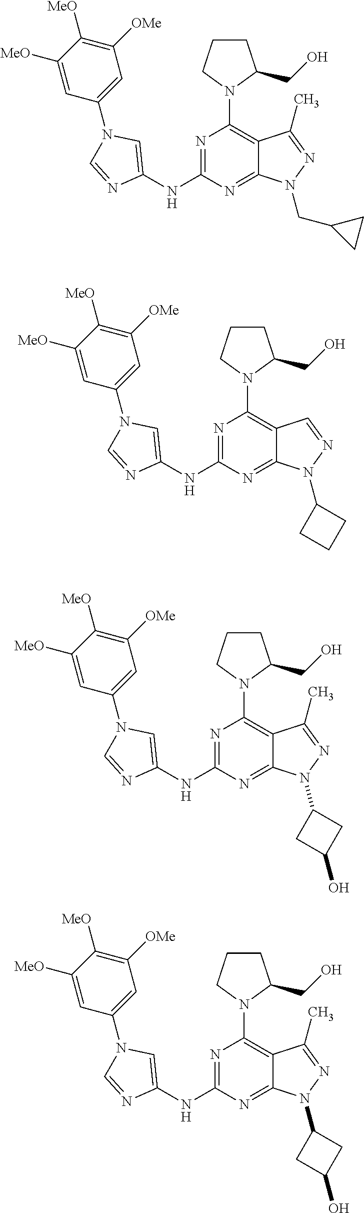 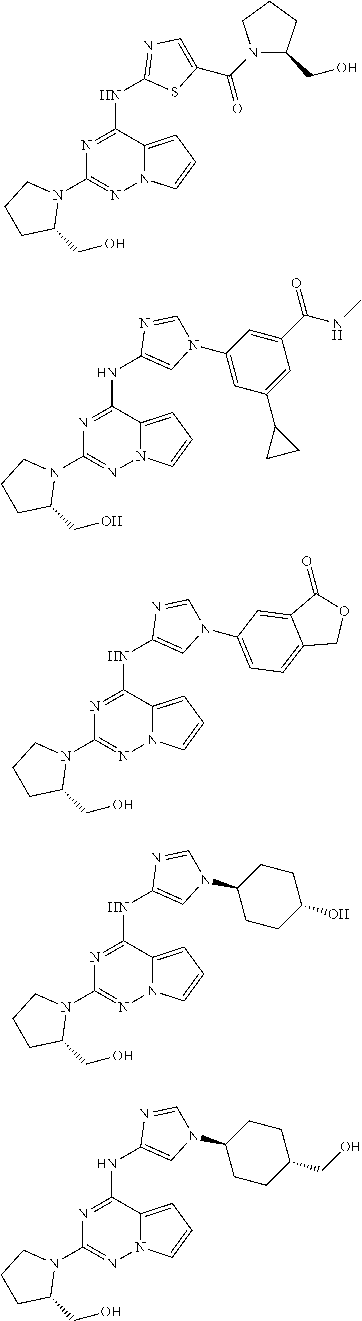 [SMILES: O=C(c1cnc(Nc2nc(N3CCC[C@H]3CO)nn3cccc23)s1)N1CCC[C@H]1CO ; CNC(=O)c1cc(C2CC2)cc(-n2cnc(Nc3nc(N4CCC[C@H]4CO)nn4cccc34)c2)c1 ; O=C1OCc2ccc(-n3cnc(Nc4nc(N5CCC[C@H]5CO)nn5cccc45)c3)cc21 ; OC[C@@H]1CCCN1c1nc(Nc2cn([C@H]3CC[C@H](O)CC3)cn2)c2cccn2n1 ; OC[C@@H]1CCCN1c1nc(Nc2cn([C@H]3CC[C@H](CO)CC3)cn2)c2cccn2n1] 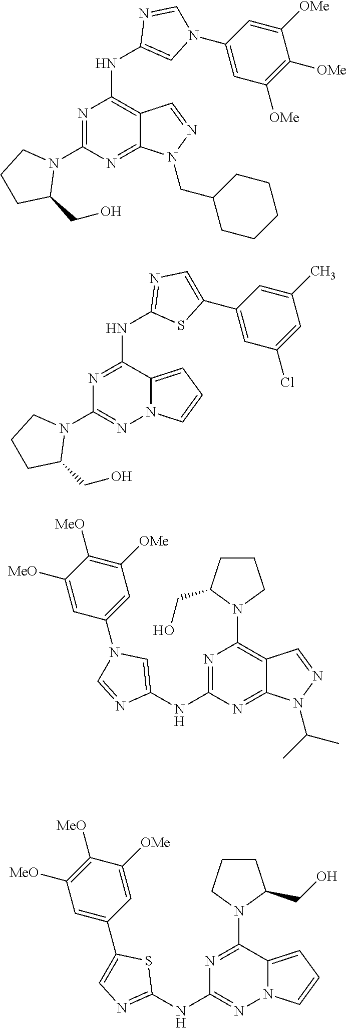 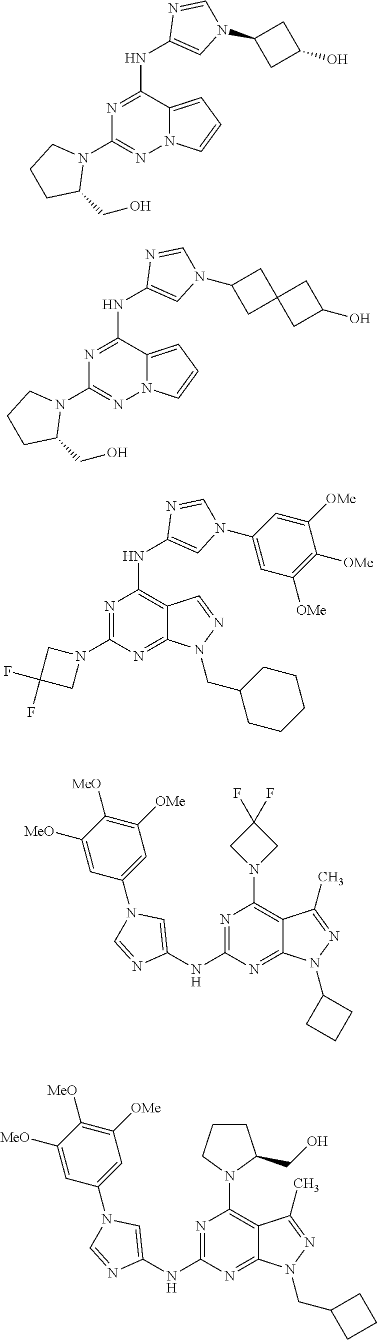 [SMILES: OC[C@@H]1CCCN1c1nc(Nc2cn([C@H]3C[C@H](O)C3)cn2)c2cccn2n1 ; OC[C@@H]1CCCN1c1nc(Nc2cn(C3CC4(CC(O)C4)C3)cn2)c2cccn2n1 ; COc1cc(-n2cnc(Nc3nc(N4CC(F)(F)C4)nc4c3cnn4CC3CCCCC3)c2)cc(OC)c1OC ; COc1cc(-n2cnc(Nc3nc(N4CC(F)(F)C4)c4c(C)nn(C5CCC5)c4n3)c2)cc(OC)c1OC ; COc1cc(-n2cnc(Nc3nc(N4CCC[C@H]4CO)c4c(C)nn(CC5CCC5)c4n3)c2)cc(OC)c1OC] 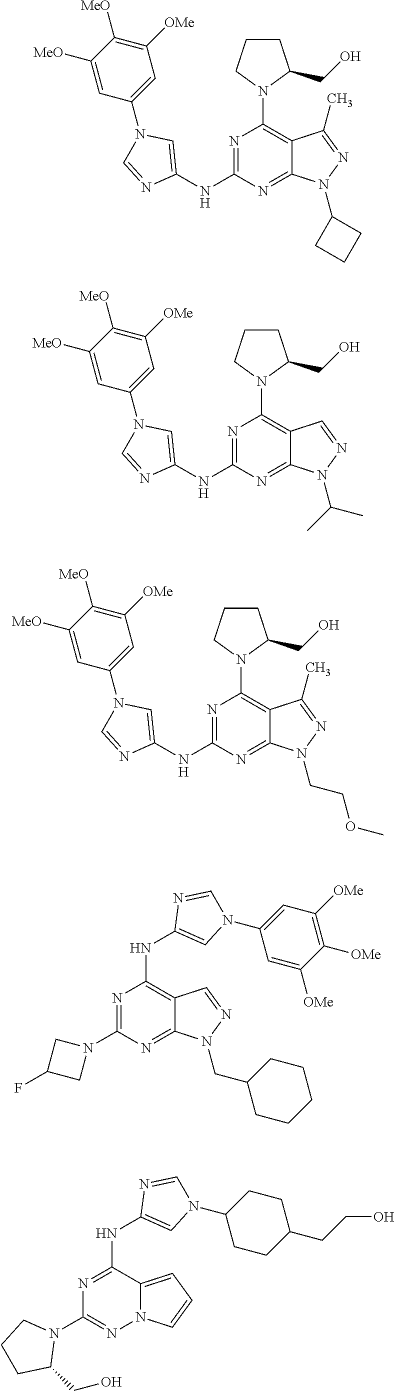 [SMILES: COc1cc(-n2cnc(Nc3nc(N4CCC[C@H]4CO)c4c(C)nn(C5CCC5)c4n3)c2)cc(OC)c1OC ; COc1cc(-n2cnc(Nc3nc(N4CCC[C@H]4CO)c4cnn(C(C)C)c4n3)c2)cc(OC)c1OC ; COCCn1nc(C)c2c(N3CCC[C@H]3CO)nc(Nc3cn(-c4cc(OC)c(OC)c(OC)c4)cn3)nc21 ; COc1cc(-n2cnc(Nc3nc(N4CC(F)C4)nc4c3cnn4CC3CCCCC3)c2)cc(OC)c1OC ; OCCC1CCC(n2cnc(Nc3nc(N4CCC[C@H]4CO)nn4cccc34)c2)CC1] 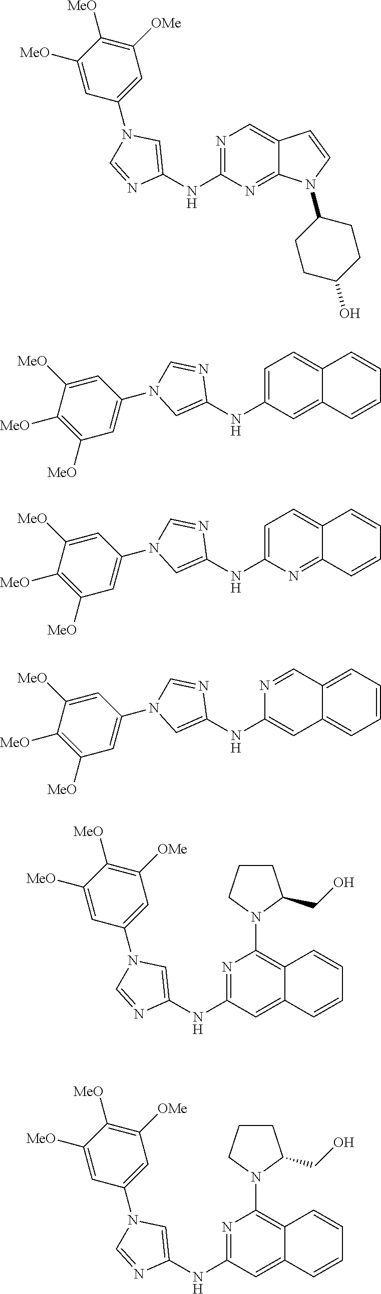 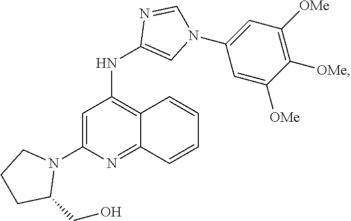 [SMILES: COc1cc(-n2cnc(Nc3cc(N4CCC[C@H]4CO)nc4ccccc34)c2)cc(OC)c1OC]
or a pharmaceutically acceptable salt or prodrug thereof.

11. A method of treating cancer, comprising orally administering to a subject in need thereof a therapeutically effective amount of a compound selected from: 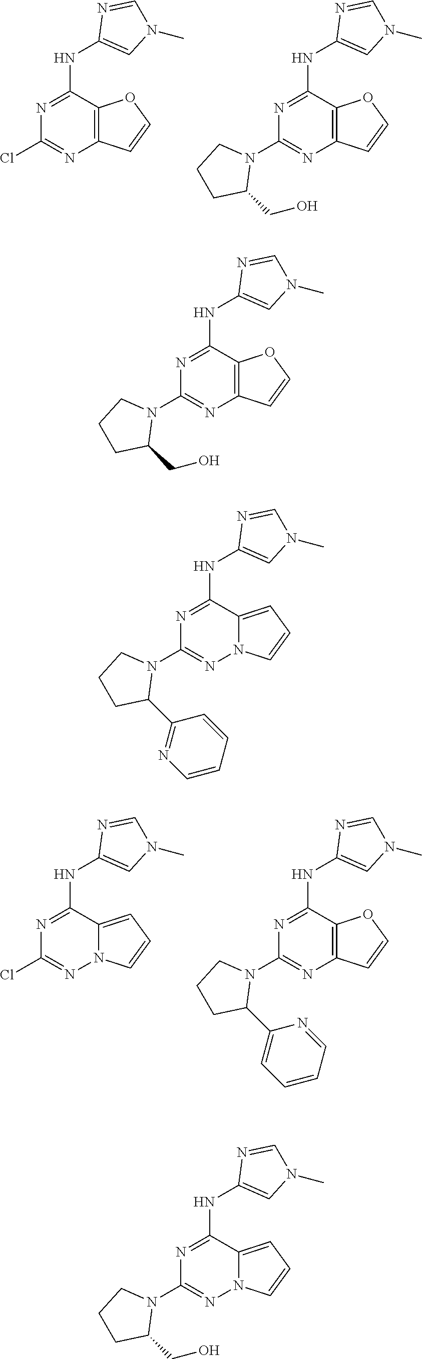 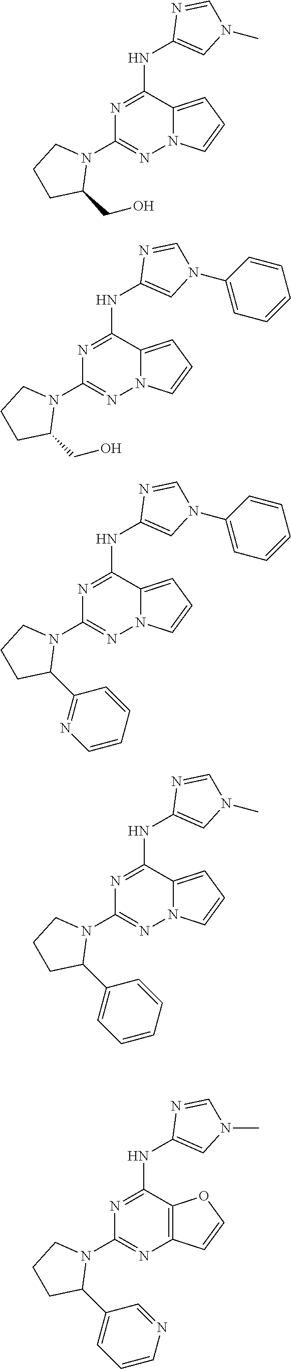 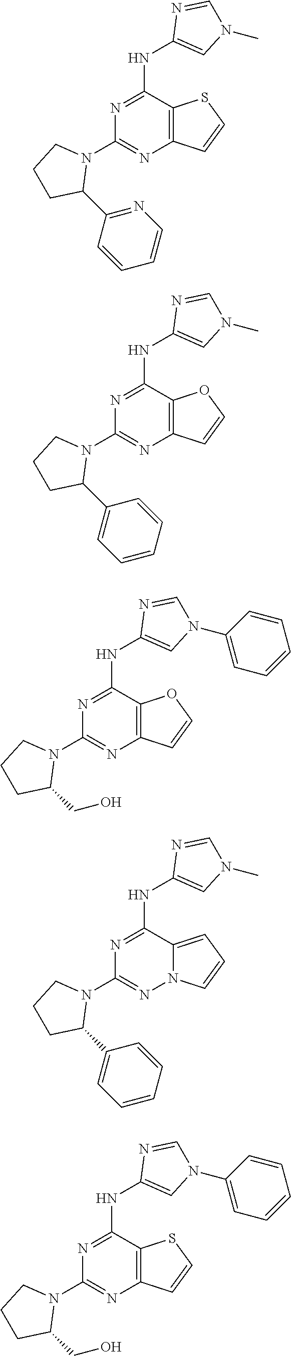 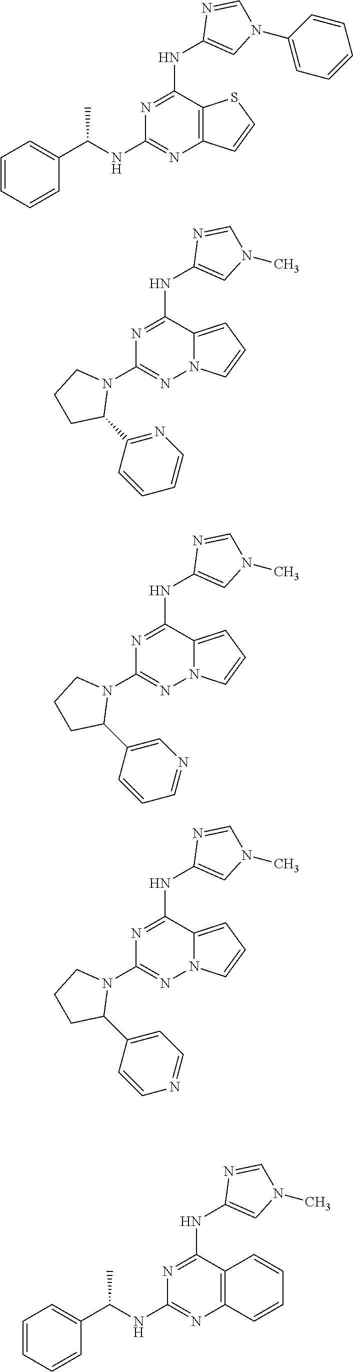 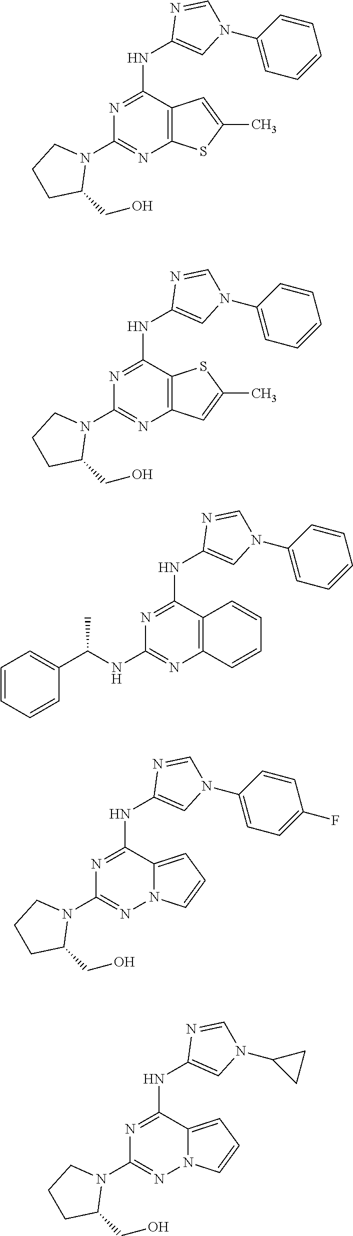 [SMILES: Cc1cc2c(Nc3cn(-c4ccccc4)cn3)nc(N3CCC[C@H]3CO)nc2s1 ; Cc1cc2nc(N3CCC[C@H]3CO)nc(Nc3cn(-c4ccccc4)cn3)c2s1 ; C[C@H](Nc1nc(Nc2cn(-c3ccccc3)cn2)c2ccccc2n1)c1ccccc1 ; OC[C@@H]1CCCN1c1nc(Nc2cn(-c3ccc(F)cc3)cn2)c2cccn2n1 ; OC[C@@H]1CCCN1c1nc(Nc2cn(C3CC3)cn2)c2cccn2n1] 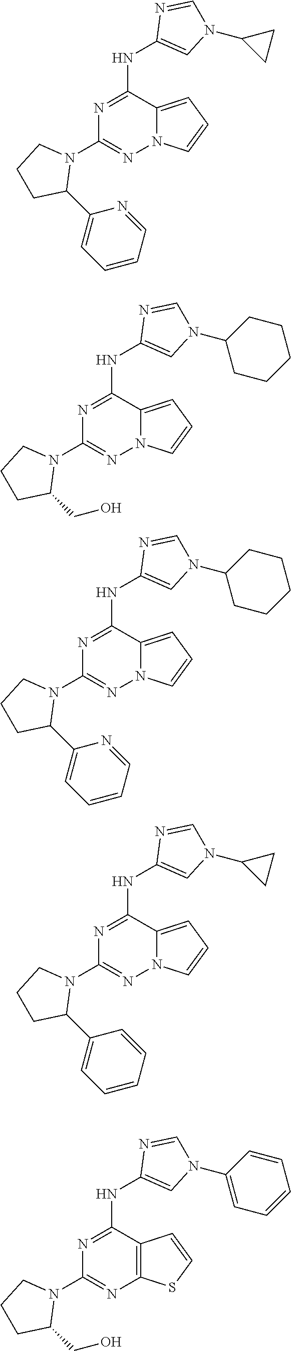 [SMILES: c1ccc(C2CCCN2c2nc(Nc3cn(C4CC4)cn3)c3cccn3n2)nc1 ; OC[C@@H]1CCCN1c1nc(Nc2cn(C3CCCCC3)cn2)c2cccn2n1 ; c1ccc(C2CCCN2c2nc(Nc3cn(C4CCCCC4)cn3)c3cccn3n2)nc1 ; c1ccc(C2CCCN2c2nc(Nc3cn(C4CC4)cn3)c3cccn3n2)cc1 ; OC[C@@H]1CCCN1c1nc(Nc2cn(-c3ccccc3)cn2)c2ccsc2n1] 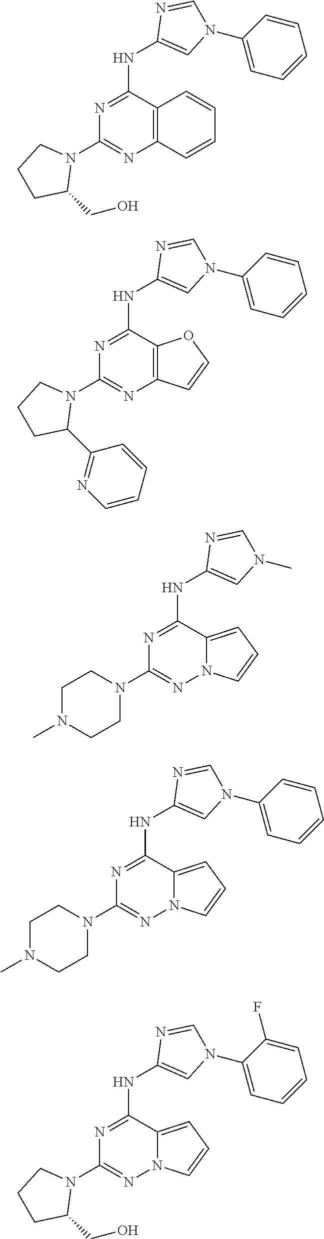 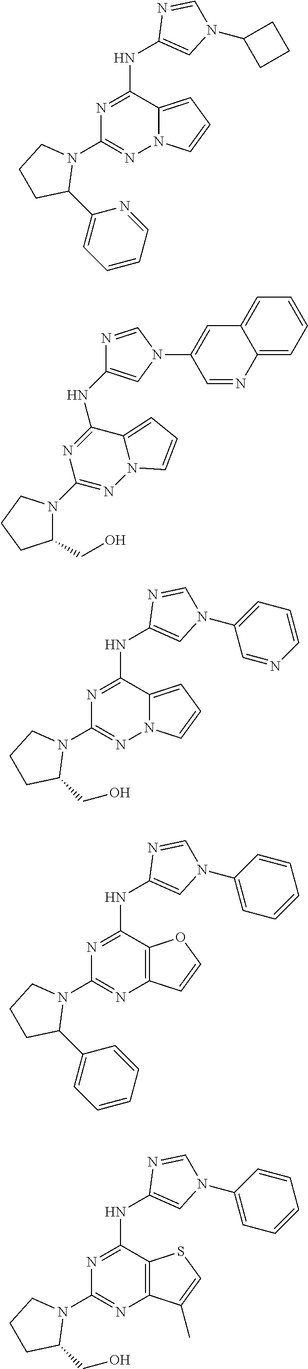 [SMILES: c1ccc(C2CCCN2c2nc(Nc3cn(C4CCC4)cn3)c3cccn3n2)nc1 ; OC[C@@H]1CCCN1c1nc(Nc2cn(-c3cnc4ccccc4c3)cn2)c2cccn2n1 ; OC[C@@H]1CCCN1c1nc(Nc2cn(-c3cccnc3)cn2)c2cccn2n1 ; c1ccc(C2CCCN2c2nc(Nc3cn(-c4ccccc4)cn3)c3occc3n2)cc1 ; Cc1csc2c(Nc3cn(-c4ccccc4)cn3)nc(N3CCC[C@H]3CO)nc12] 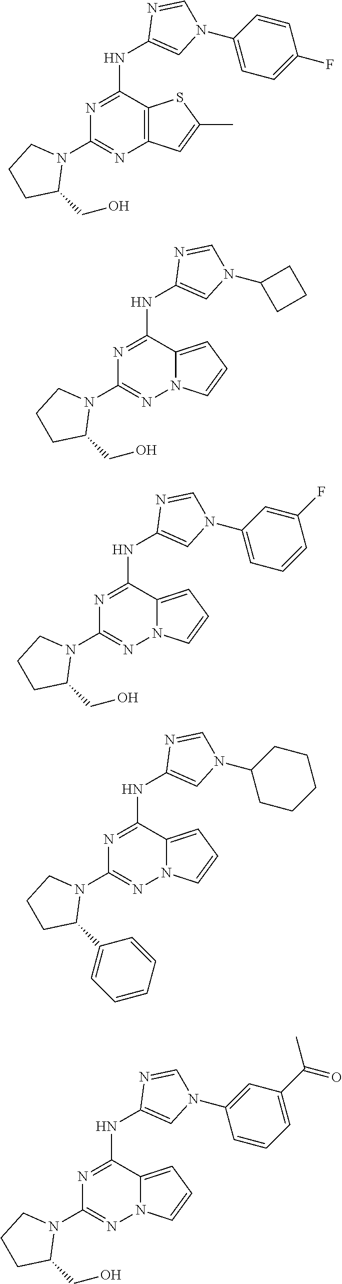 [SMILES: Cc1cc2nc(N3CCC[C@H]3CO)nc(Nc3cn(-c4ccc(F)cc4)cn3)c2s1 ; OC[C@@H]1CCCN1c1nc(Nc2cn(C3CCC3)cn2)c2cccn2n1 ; OC[C@@H]1CCCN1c1nc(Nc2cn(-c3cccc(F)c3)cn2)c2cccn2n1 ; c1ccc([C@@H]2CCCN2c2nc(Nc3cn(C4CCCCC4)cn3)c3cccn3n2)cc1 ; CC(=O)c1cccc(-n2cnc(Nc3nc(N4CCC[C@H]4CO)nn4cccc34)c2)c1] 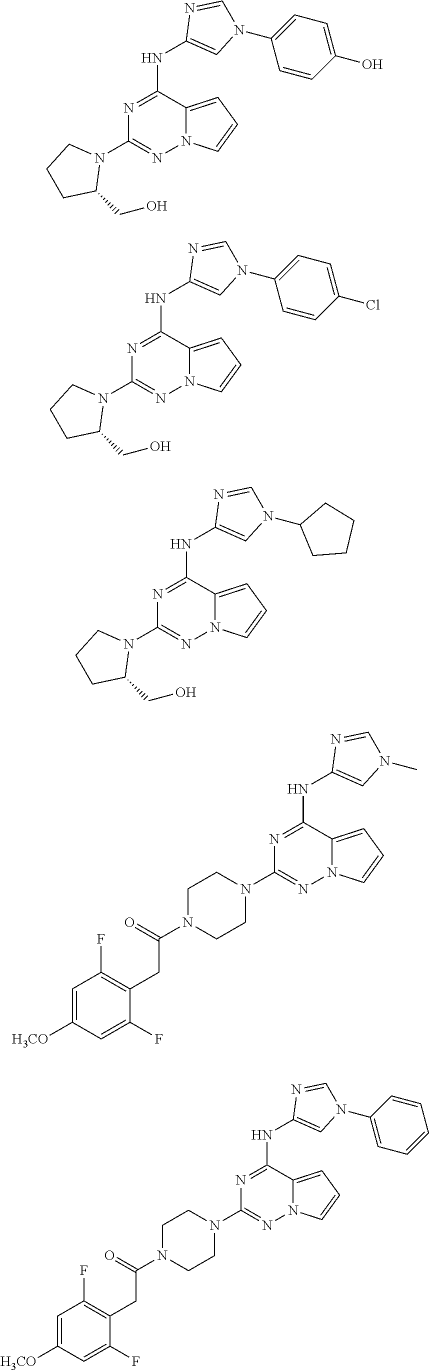 [SMILES: OC[C@@H]1CCCN1c1nc(Nc2cn(-c3ccc(O)cc3)cn2)c2cccn2n1 ; OC[C@@H]1CCCN1c1nc(Nc2cn(-c3ccc(Cl)cc3)cn2)c2cccn2n1 ; OC[C@@H]1CCCN1c1nc(Nc2cn(C3CCCC3)cn2)c2cccn2n1 ; COc1cc(F)c(CC(=O)N2CCN(c3nc(Nc4cn(C)cn4)c4cccn4n3)CC2)c(F)c1 ; COc1cc(F)c(CC(=O)N2CCN(c3nc(Nc4cn(-c5ccccc5)cn4)c4cccn4n3)CC2)c(F)c1] 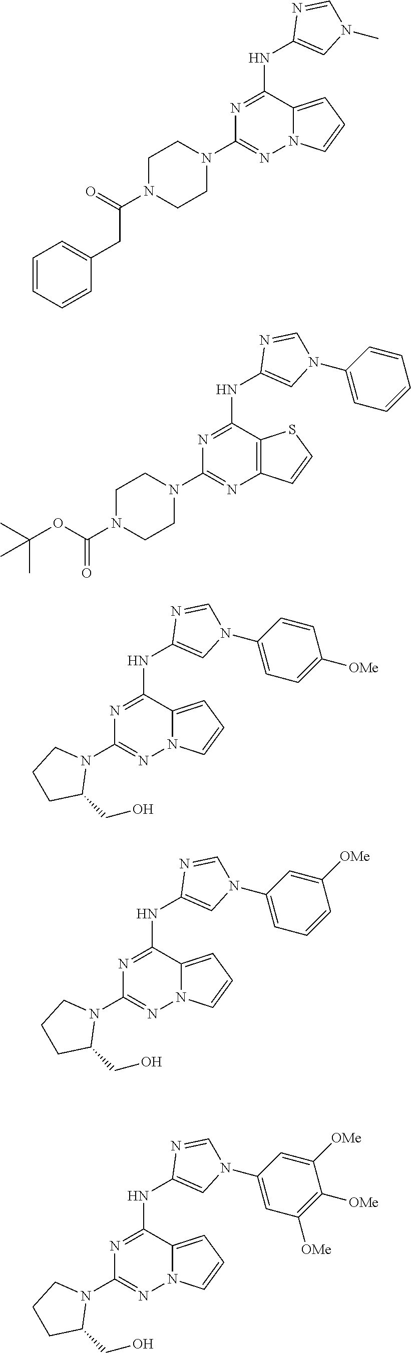 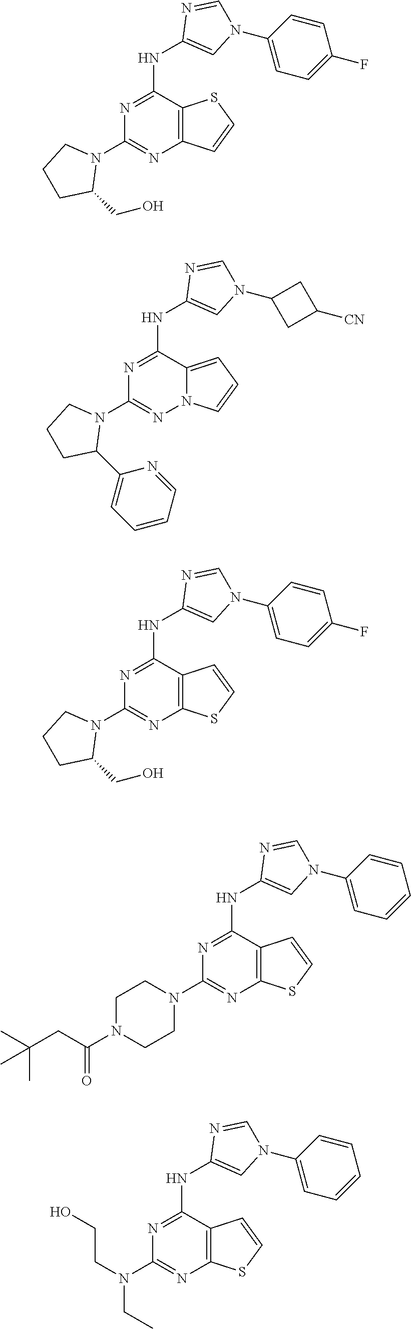 [SMILES: OC[C@@H]1CCCN1c1nc(Nc2cn(-c3ccc(F)cc3)cn2)c2sccc2n1 ; N#CC1CC(n2cnc(Nc3nc(N4CCCC4c4ccccn4)nn4cccc34)c2)C1 ; OC[C@@H]1CCCN1c1nc(Nc2cn(-c3ccc(F)cc3)cn2)c2ccsc2n1 ; CC(C)(C)CC(=O)N1CCN(c2nc(Nc3cn(-c4ccccc4)cn3)c3ccsc3n2)CC1 ; CCN(CCO)c1nc(Nc2cn(-c3ccccc3)cn2)c2ccsc2n1] 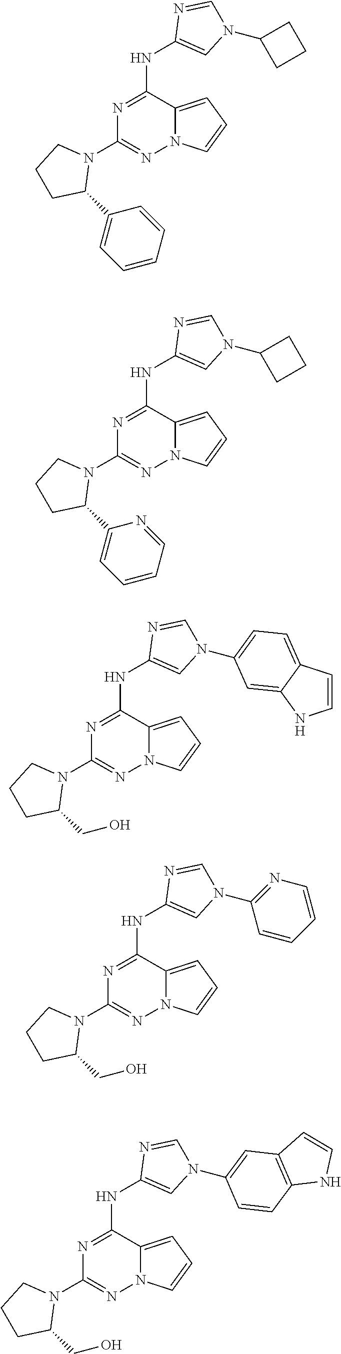 [SMILES: c1ccc([C@@H]2CCCN2c2nc(Nc3cn(C4CCC4)cn3)c3cccn3n2)cc1 ; c1ccc([C@@H]2CCCN2c2nc(Nc3cn(C4CCC4)cn3)c3cccn3n2)nc1 ; OC[C@@H]1CCCN1c1nc(Nc2cn(-c3ccc4cc[nH]c4c3)cn2)c2cccn2n1 ; OC[C@@H]1CCCN1c1nc(Nc2cn(-c3ccccn3)cn2)c2cccn2n1 ; OC[C@@H]1CCCN1c1nc(Nc2cn(-c3ccc4[nH]ccc4c3)cn2)c2cccn2n1] 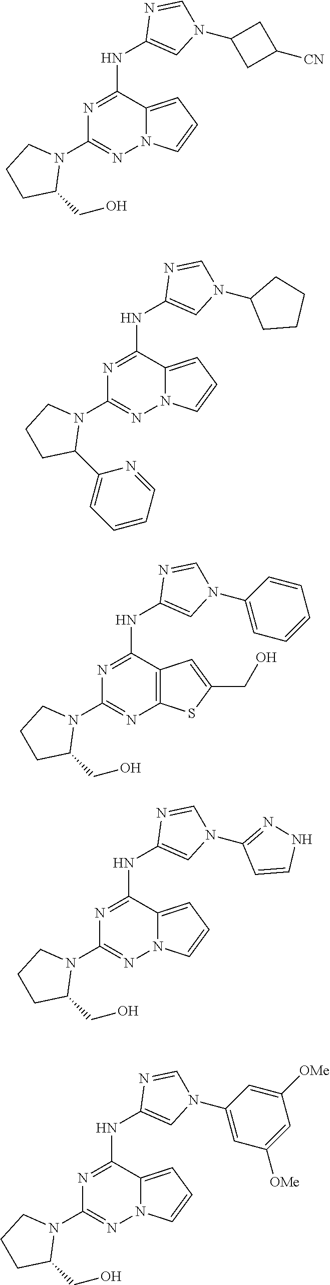 [SMILES: N#CC1CC(n2cnc(Nc3nc(N4CCC[C@H]4CO)nn4cccc34)c2)C1 ; c1ccc(C2CCCN2c2nc(Nc3cn(C4CCCC4)cn3)c3cccn3n2)nc1 ; OCc1cc2c(Nc3cn(-c4ccccc4)cn3)nc(N3CCC[C@H]3CO)nc2s1 ; OC[C@@H]1CCCN1c1nc(Nc2cn(-c3cc[nH]n3)cn2)c2cccn2n1 ; COc1cc(OC)cc(-n2cnc(Nc3nc(N4CCC[C@H]4CO)nn4cccc34)c2)c1] 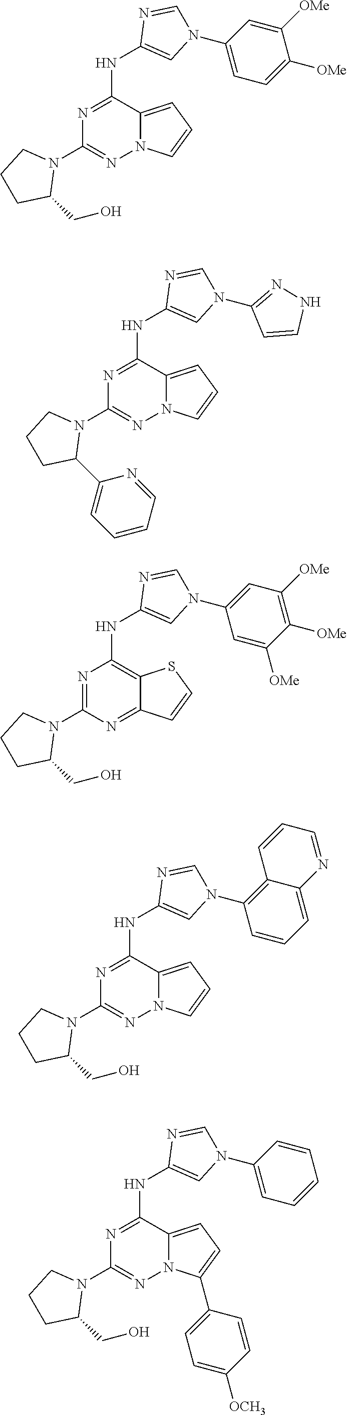 [SMILES: COc1ccc(-n2cnc(Nc3nc(N4CCC[C@H]4CO)nn4cccc34)c2)cc1OC ; c1ccc(C2CCCN2c2nc(Nc3cn(-c4cc[nH]n4)cn3)c3cccn3n2)nc1 ; COc1cc(-n2cnc(Nc3nc(N4CCC[C@H]4CO)nc4ccsc34)c2)cc(OC)c1OC ; OC[C@@H]1CCCN1c1nc(Nc2cn(-c3cccc4ncccc34)cn2)c2cccn2n1 ; COc1ccc(-c2ccc3c(Nc4cn(-c5ccccc5)cn4)nc(N4CCC[C@H]4CO)nn23)cc1] 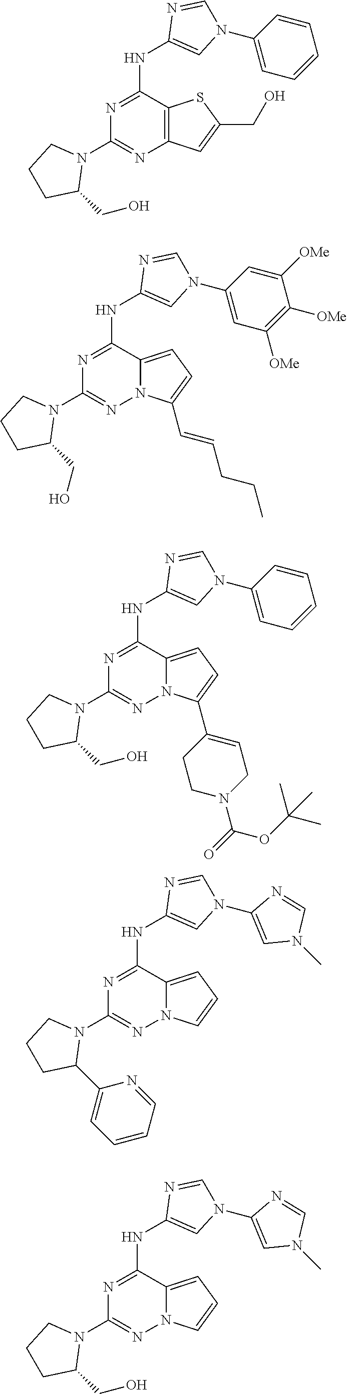 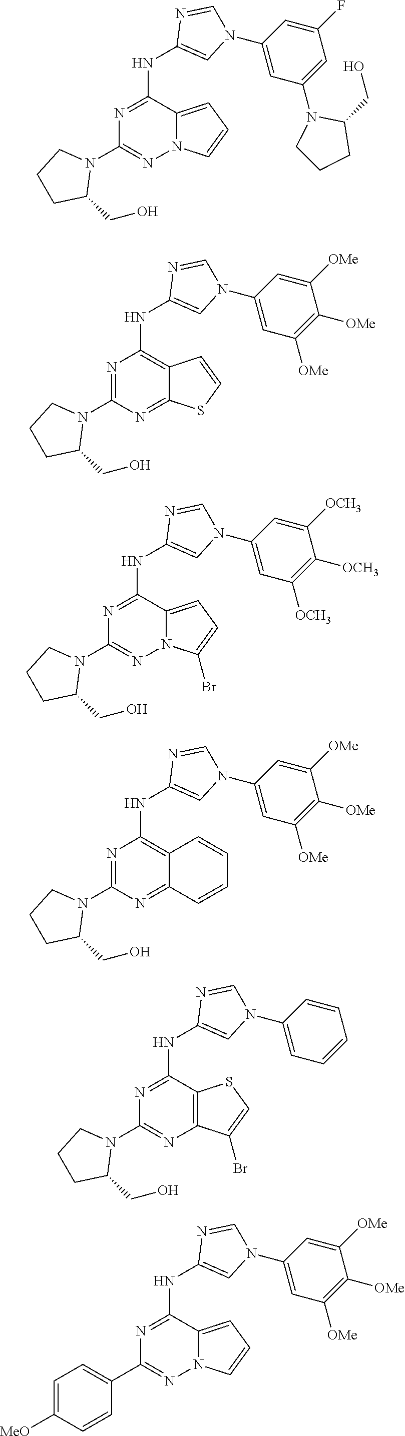 [SMILES: OC[C@@H]1CCCN1c1cc(F)cc(-n2cnc(Nc3nc(N4CCC[C@H]4CO)nn4cccc34)c2)c1 ; COc1cc(-n2cnc(Nc3nc(N4CCC[C@H]4CO)nc4sccc34)c2)cc(OC)c1OC ; COc1cc(-n2cnc(Nc3nc(N4CCC[C@H]4CO)nn4c(Br)ccc34)c2)cc(OC)c1OC ; COc1cc(-n2cnc(Nc3nc(N4CCC[C@H]4CO)nc4ccccc34)c2)cc(OC)c1OC ; OC[C@@H]1CCCN1c1nc(Nc2cn(-c3ccccc3)cn2)c2scc(Br)c2n1 ; COc1ccc(-c2nc(Nc3cn(-c4cc(OC)c(OC)c(OC)c4)cn3)c3cccn3n2)cc1] 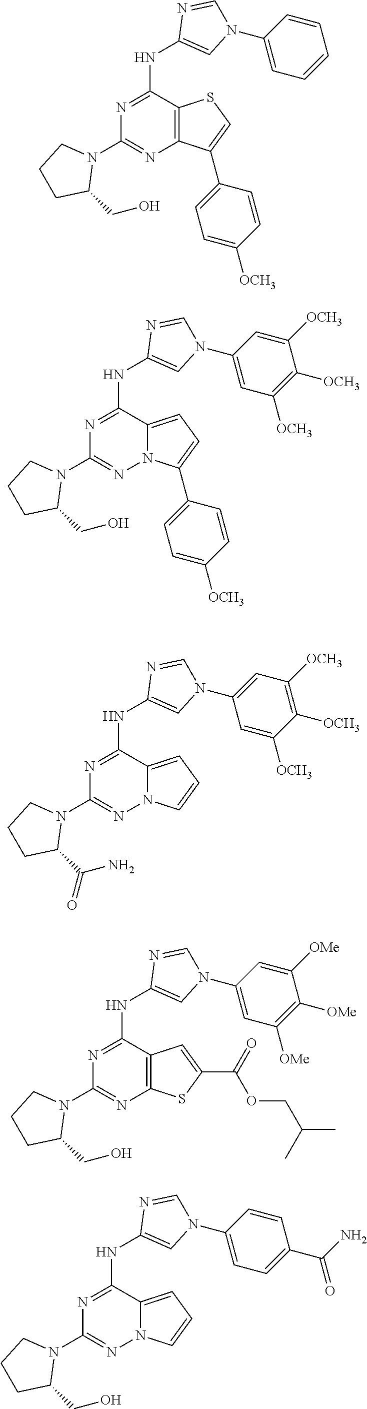 [SMILES: COc1ccc(-c2csc3c(Nc4cn(-c5ccccc5)cn4)nc(N4CCC[C@H]4CO)nc23)cc1 ; COc1ccc(-c2ccc3c(Nc4cn(-c5cc(OC)c(OC)c(OC)c5)cn4)nc(N4CCC[C@H]4CO)nn23)cc1 ; COc1cc(-n2cnc(Nc3nc(N4CCC[C@H]4C(N)=O)nn4cccc34)c2)cc(OC)c1OC ; COc1cc(-n2cnc(Nc3nc(N4CCC[C@H]4CO)nc4sc(C(=O)OCC(C)C)cc34)c2)cc(OC)c1OC ; NC(=O)c1ccc(-n2cnc(Nc3nc(N4CCC[C@H]4CO)nn4cccc34)c2)cc1] 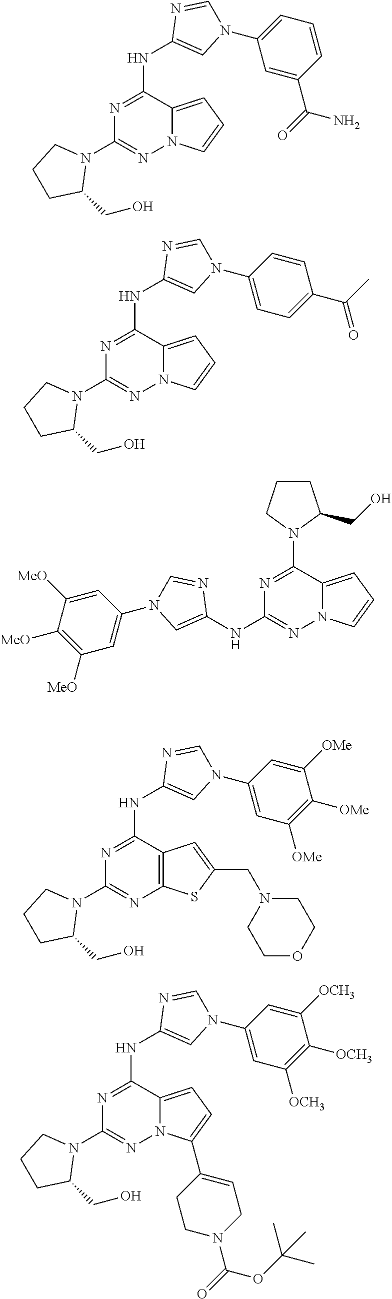 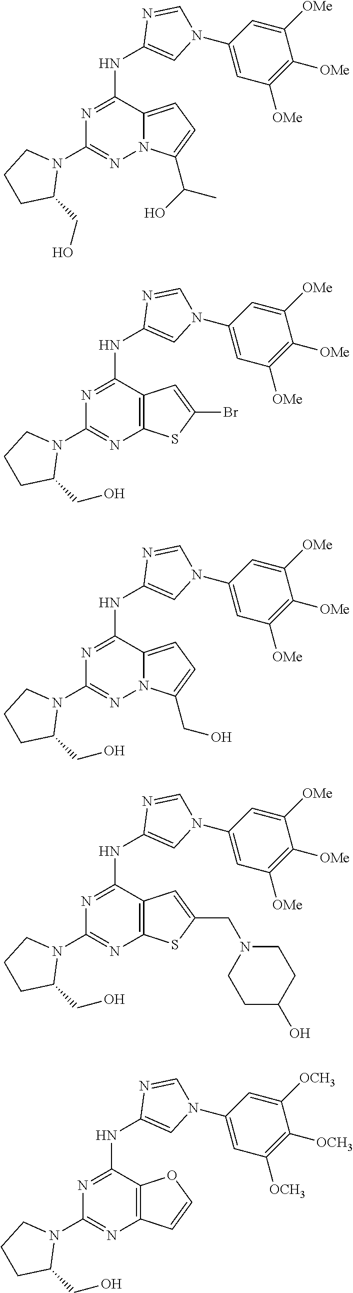 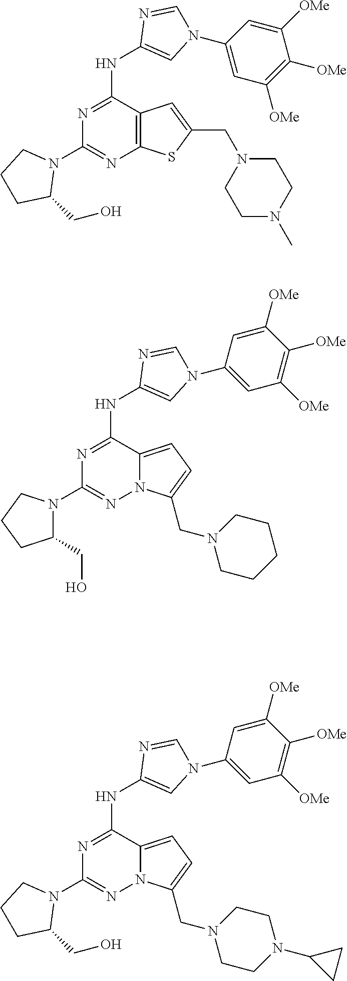 [SMILES: COc1cc(-n2cnc(Nc3nc(N4CCC[C@H]4CO)nc4sc(CN5CCN(C)CC5)cc34)c2)cc(OC)c1OC ; COc1cc(-n2cnc(Nc3nc(N4CCC[C@H]4CO)nn4c(CN5CCCCC5)ccc34)c2)cc(OC)c1OC ; COc1cc(-n2cnc(Nc3nc(N4CCC[C@H]4CO)nn4c(CN5CCN(C6CC6)CC5)ccc34)c2)cc(OC)c1OC] 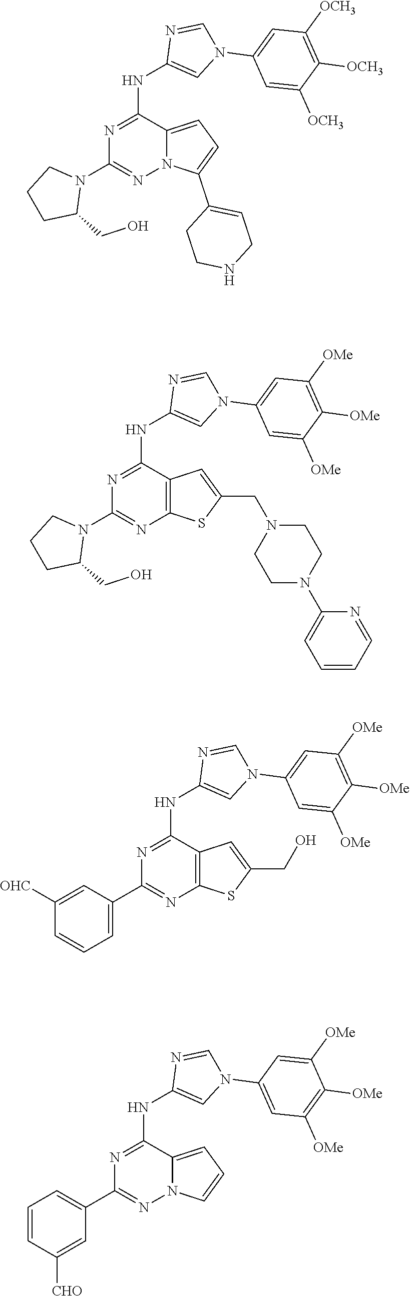 [SMILES: COc1cc(-n2cnc(Nc3nc(N4CCC[C@H]4CO)nn4c(C5=CCNCC5)ccc34)c2)cc(OC)c1OC ; COc1cc(-n2cnc(Nc3nc(N4CCC[C@H]4CO)nc4sc(CN5CCN(c6ccccn6)CC5)cc34)c2)cc(OC)c1OC ; COc1cc(-n2cnc(Nc3nc(-c4cccc(C=O)c4)nc4sc(CO)cc34)c2)cc(OC)c1OC ; COc1cc(-n2cnc(Nc3nc(-c4cccc(C=O)c4)nn4cccc34)c2)cc(OC)c1OC] 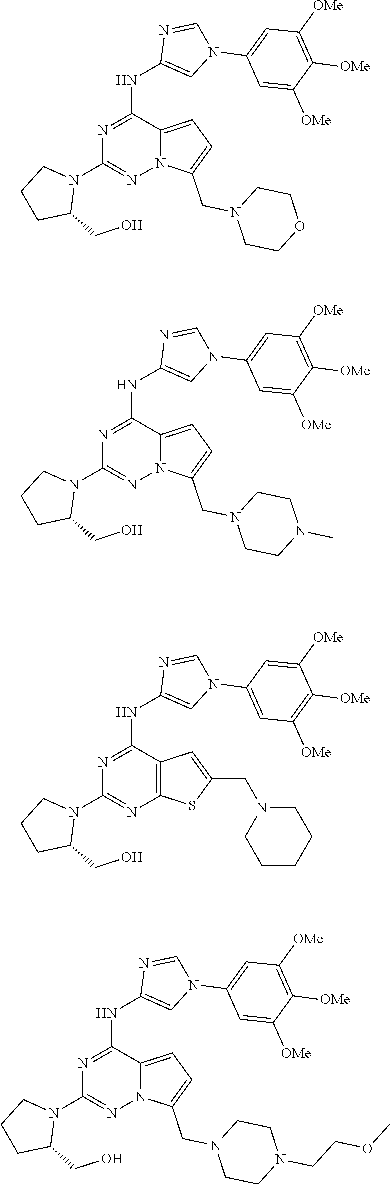 [SMILES: COc1cc(-n2cnc(Nc3nc(N4CCC[C@H]4CO)nn4c(CN5CCOCC5)ccc34)c2)cc(OC)c1OC ; COc1cc(-n2cnc(Nc3nc(N4CCC[C@H]4CO)nn4c(CN5CCN(C)CC5)ccc34)c2)cc(OC)c1OC ; COc1cc(-n2cnc(Nc3nc(N4CCC[C@H]4CO)nc4sc(CN5CCCCC5)cc34)c2)cc(OC)c1OC ; COCCN1CCN(Cc2ccc3c(Nc4cn(-c5cc(OC)c(OC)c(OC)c5)cn4)nc(N4CCC[C@H]4CO)nn23)CC1] 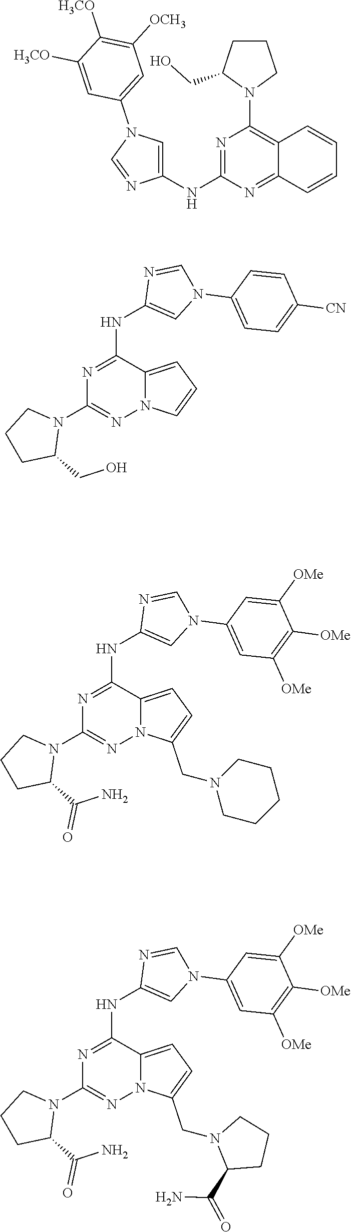 [SMILES: COc1cc(-n2cnc(Nc3nc(N4CCC[C@H]4CO)c4ccccc4n3)c2)cc(OC)c1OC ; N#Cc1ccc(-n2cnc(Nc3nc(N4CCC[C@H]4CO)nn4cccc34)c2)cc1 ; COc1cc(-n2cnc(Nc3nc(N4CCC[C@H]4C(N)=O)nn4c(CN5CCCCC5)ccc34)c2)cc(OC)c1OC ; COc1cc(-n2cnc(Nc3nc(N4CCC[C@H]4C(N)=O)nn4c(CN5CCC[C@H]5C(N)=O)ccc34)c2)cc(OC)c1OC] 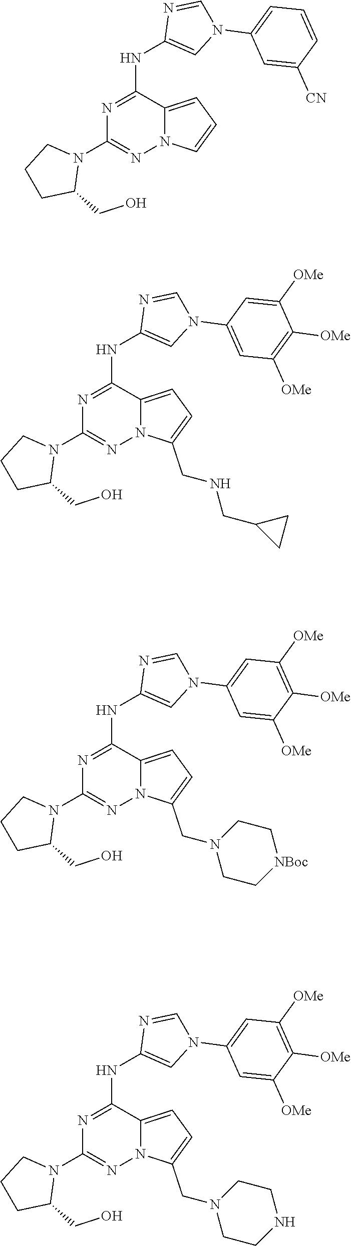 [SMILES: N#Cc1cccc(-n2cnc(Nc3nc(N4CCC[C@H]4CO)nn4cccc34)c2)c1 ; COc1cc(-n2cnc(Nc3nc(N4CCC[C@H]4CO)nn4c(CNCC5CC5)ccc34)c2)cc(OC)c1OC ; COc1cc(-n2cnc(Nc3nc(N4CCC[C@H]4CO)nn4c(CN5CCN(C(=O)OC(C)(C)C)CC5)ccc34)c2)cc(OC)c1OC ; COc1cc(-n2cnc(Nc3nc(N4CCC[C@H]4CO)nn4c(CN5CCNCC5)ccc34)c2)cc(OC)c1OC] 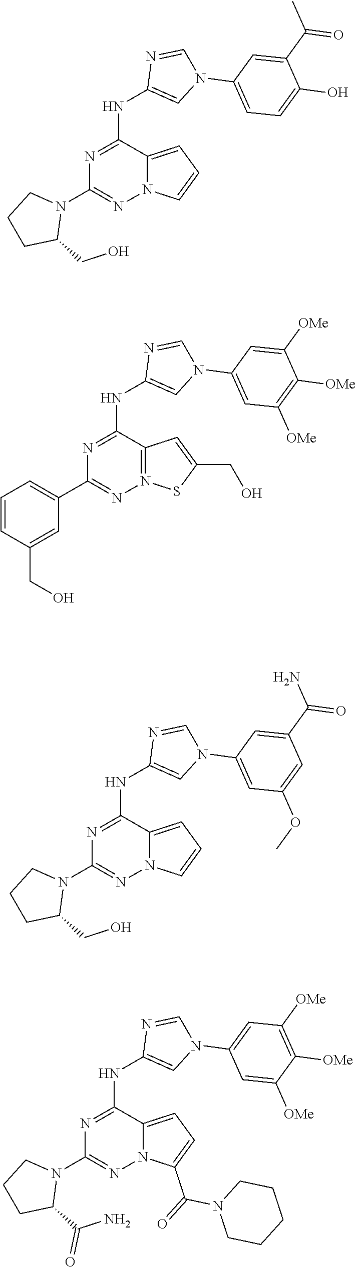 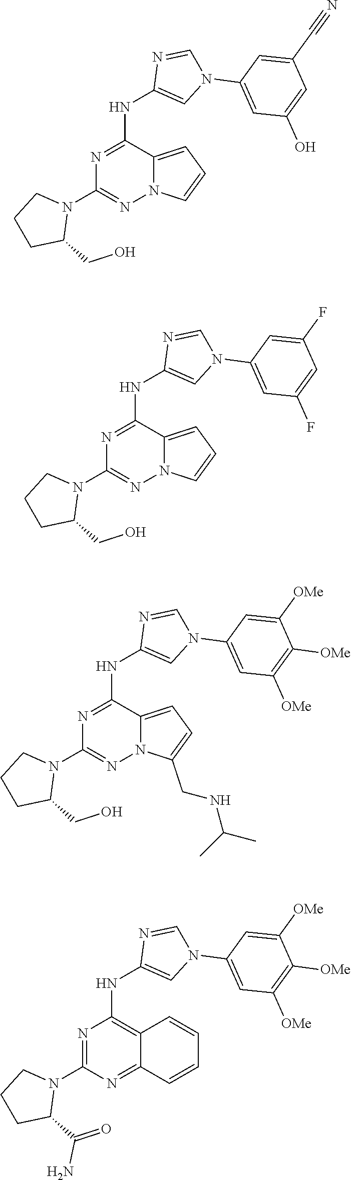 [SMILES: N#Cc1cc(O)cc(-n2cnc(Nc3nc(N4CCC[C@H]4CO)nn4cccc34)c2)c1 ; OC[C@@H]1CCCN1c1nc(Nc2cn(-c3cc(F)cc(F)c3)cn2)c2cccn2n1 ; COc1cc(-n2cnc(Nc3nc(N4CCC[C@H]4CO)nn4c(CNC(C)C)ccc34)c2)cc(OC)c1OC ; COc1cc(-n2cnc(Nc3nc(N4CCC[C@H]4C(N)=O)nc4ccccc34)c2)cc(OC)c1OC] 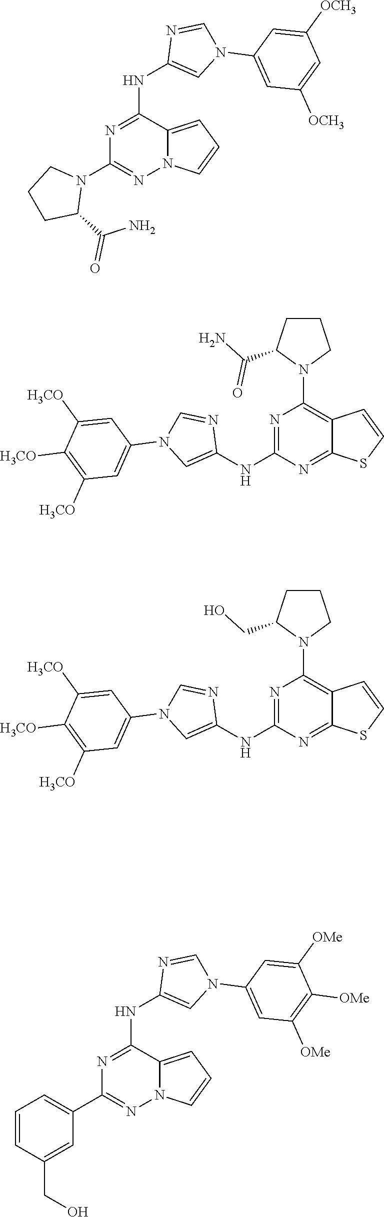 [SMILES: COc1cc(OC)cc(-n2cnc(Nc3nc(N4CCC[C@H]4C(N)=O)nn4cccc34)c2)c1 ; COc1cc(-n2cnc(Nc3nc(N4CCC[C@H]4C(N)=O)c4ccsc4n3)c2)cc(OC)c1OC ; COc1cc(-n2cnc(Nc3nc(N4CCC[C@H]4CO)c4ccsc4n3)c2)cc(OC)c1OC ; COc1cc(-n2cnc(Nc3nc(-c4cccc(CO)c4)nn4cccc34)c2)cc(OC)c1OC] 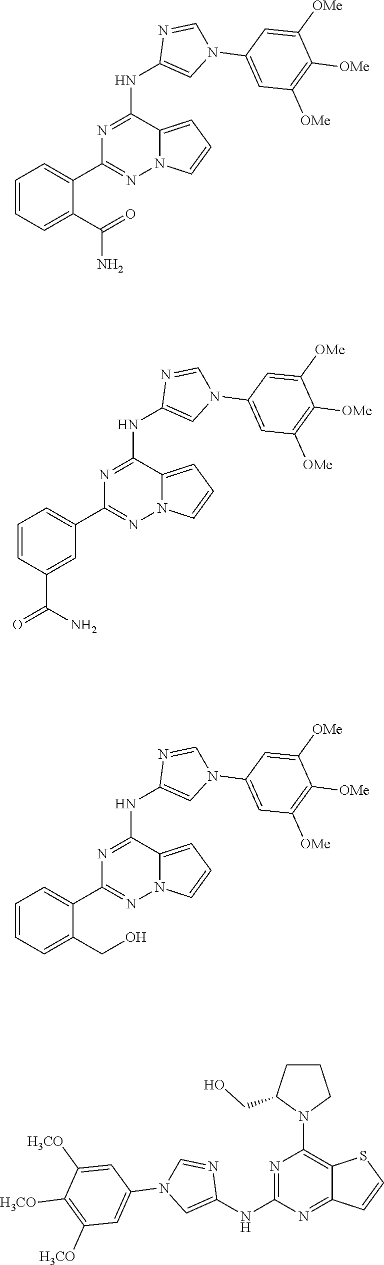 [SMILES: COc1cc(-n2cnc(Nc3nc(-c4ccccc4C(N)=O)nn4cccc34)c2)cc(OC)c1OC ; COc1cc(-n2cnc(Nc3nc(-c4cccc(C(N)=O)c4)nn4cccc34)c2)cc(OC)c1OC ; COc1cc(-n2cnc(Nc3nc(-c4ccccc4CO)nn4cccc34)c2)cc(OC)c1OC ; COc1cc(-n2cnc(Nc3nc(N4CCC[C@H]4CO)c4sccc4n3)c2)cc(OC)c1OC] 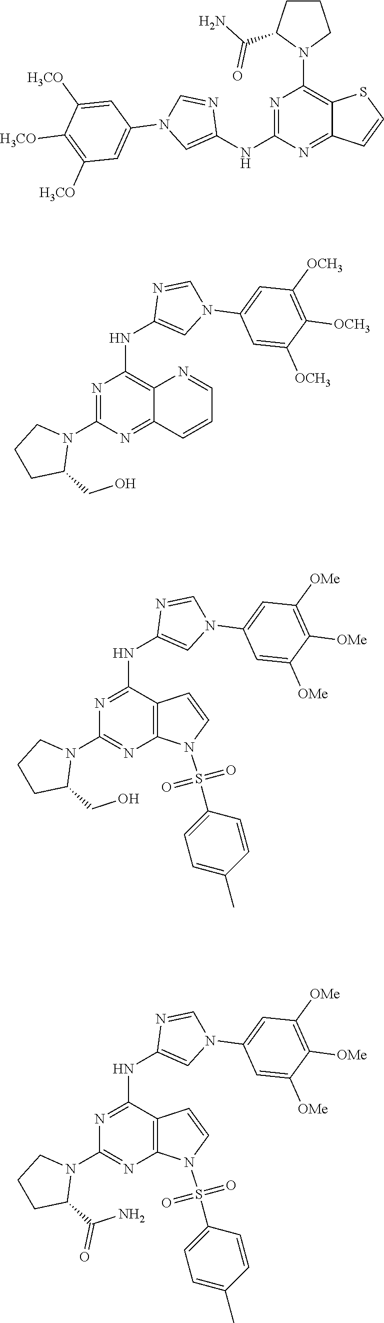 [SMILES: COc1cc(-n2cnc(Nc3nc(N4CCC[C@H]4C(N)=O)c4sccc4n3)c2)cc(OC)c1OC ; COc1cc(-n2cnc(Nc3nc(N4CCC[C@H]4CO)nc4cccnc34)c2)cc(OC)c1OC ; COc1cc(-n2cnc(Nc3nc(N4CCC[C@H]4CO)nc4c3ccn4S(=O)(=O)c3ccc(C)cc3)c2)cc(OC)c1OC ; COc1cc(-n2cnc(Nc3nc(N4CCC[C@H]4C(N)=O)nc4c3ccn4S(=O)(=O)c3ccc(C)cc3)c2)cc(OC)c1OC] 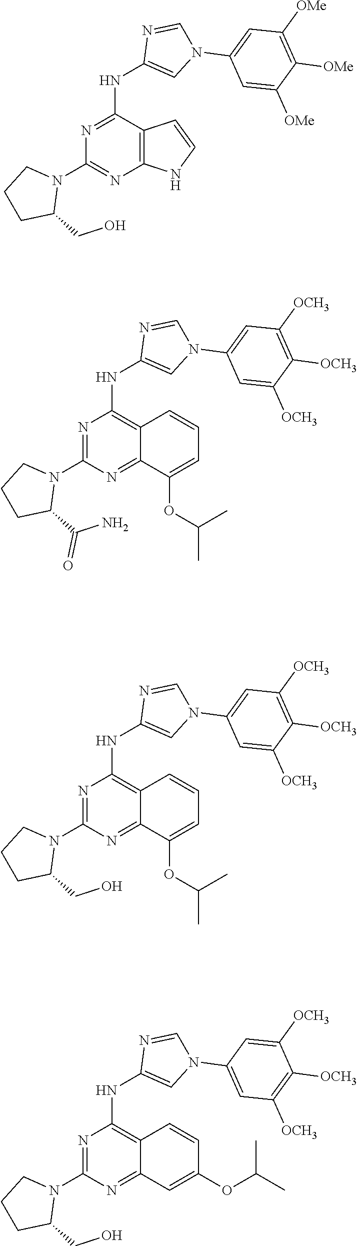 [SMILES: COc1cc(-n2cnc(Nc3nc(N4CCC[C@H]4CO)nc4[nH]ccc34)c2)cc(OC)c1OC ; COc1cc(-n2cnc(Nc3nc(N4CCC[C@H]4C(N)=O)nc4c(OC(C)C)cccc34)c2)cc(OC)c1OC ; COc1cc(-n2cnc(Nc3nc(N4CCC[C@H]4CO)nc4c(OC(C)C)cccc34)c2)cc(OC)c1OC ; COc1cc(-n2cnc(Nc3nc(N4CCC[C@H]4CO)nc4cc(OC(C)C)ccc34)c2)cc(OC)c1OC] 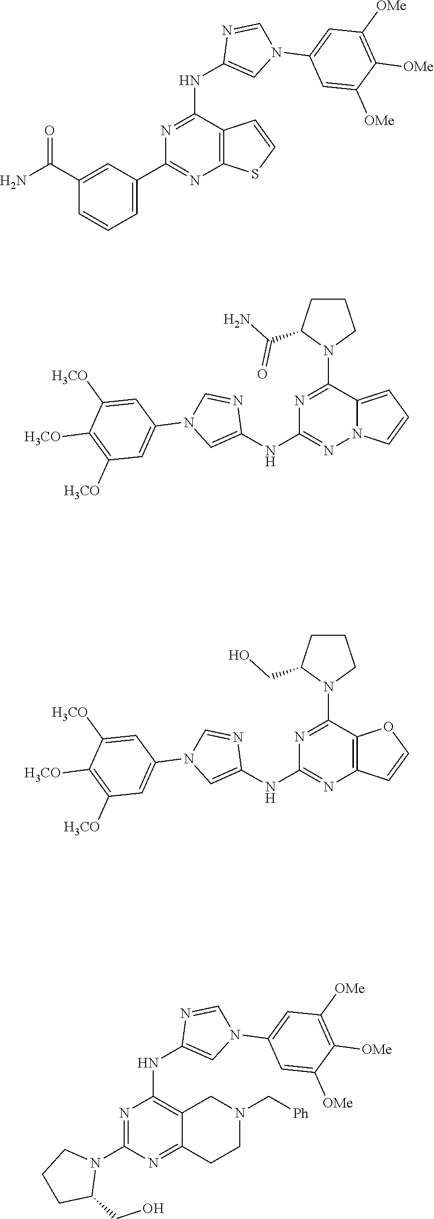 [SMILES: COc1cc(-n2cnc(Nc3nc(-c4cccc(C(N)=O)c4)nc4sccc34)c2)cc(OC)c1OC ; COc1cc(-n2cnc(Nc3nc(N4CCC[C@H]4C(N)=O)c4cccn4n3)c2)cc(OC)c1OC ; COc1cc(-n2cnc(Nc3nc(N4CCC[C@H]4CO)c4occc4n3)c2)cc(OC)c1OC ; COc1cc(-n2cnc(Nc3nc(N4CCC[C@H]4CO)nc4c3CN(Cc3ccccc3)CC4)c2)cc(OC)c1OC] 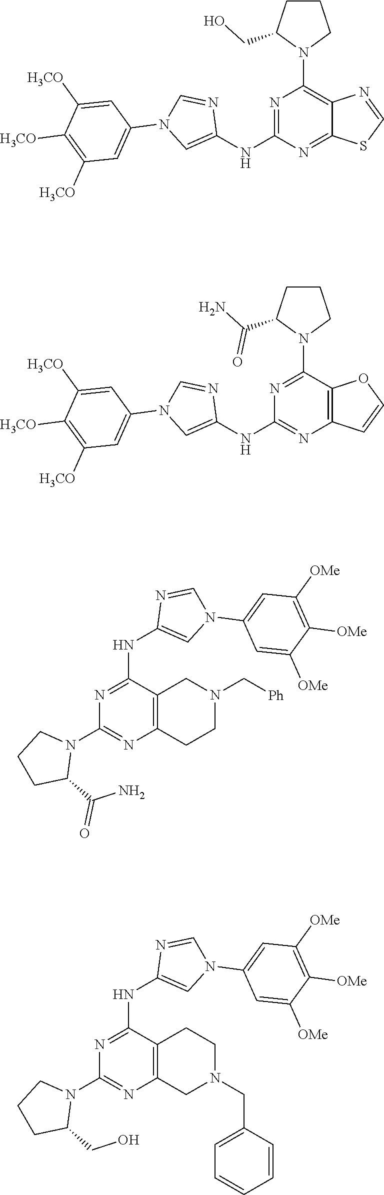 [SMILES: COc1cc(-n2cnc(Nc3nc(N4CCC[C@H]4CO)c4ncsc4n3)c2)cc(OC)c1OC ; COc1cc(-n2cnc(Nc3nc(N4CCC[C@H]4C(N)=O)c4occc4n3)c2)cc(OC)c1OC ; COc1cc(-n2cnc(Nc3nc(N4CCC[C@H]4C(N)=O)nc4c3CN(Cc3ccccc3)CC4)c2)cc(OC)c1OC ; COc1cc(-n2cnc(Nc3nc(N4CCC[C@H]4CO)nc4c3CCN(Cc3ccccc3)C4)c2)cc(OC)c1OC] 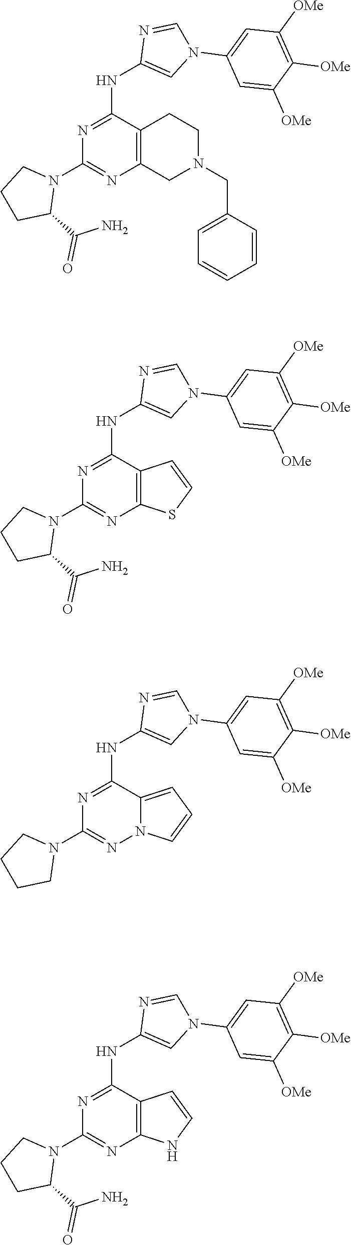 [SMILES: COc1cc(-n2cnc(Nc3nc(N4CCC[C@H]4C(N)=O)nc4c3CCN(Cc3ccccc3)C4)c2)cc(OC)c1OC ; COc1cc(-n2cnc(Nc3nc(N4CCC[C@H]4C(N)=O)nc4sccc34)c2)cc(OC)c1OC ; COc1cc(-n2cnc(Nc3nc(N4CCCC4)nn4cccc34)c2)cc(OC)c1OC ; COc1cc(-n2cnc(Nc3nc(N4CCC[C@H]4C(N)=O)nc4[nH]ccc34)c2)cc(OC)c1OC] 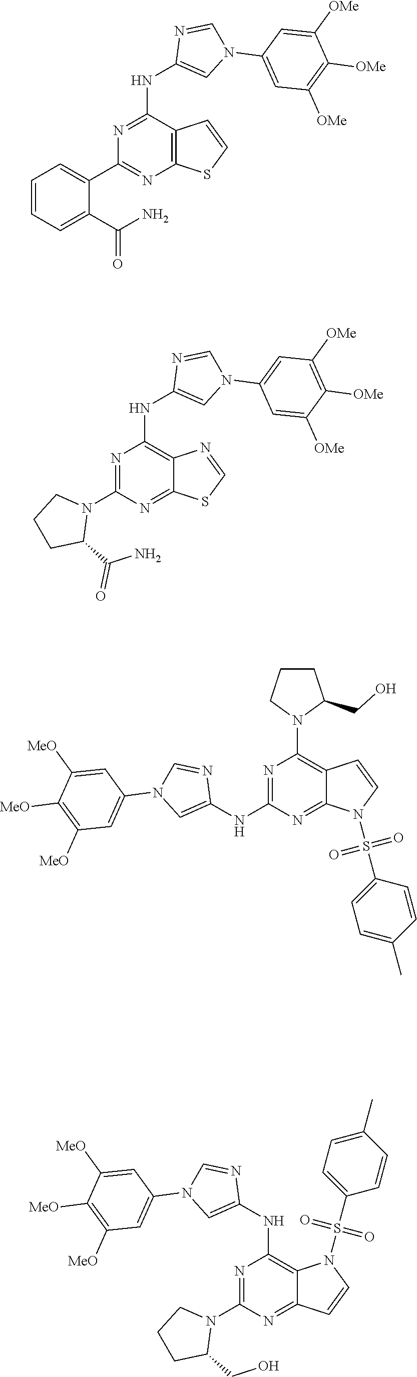 [SMILES: COc1cc(-n2cnc(Nc3nc(-c4ccccc4C(N)=O)nc4sccc34)c2)cc(OC)c1OC ; COc1cc(-n2cnc(Nc3nc(N4CCC[C@H]4C(N)=O)nc4scnc34)c2)cc(OC)c1OC ; COc1cc(-n2cnc(Nc3nc(N4CCC[C@H]4CO)c4ccn(S(=O)(=O)c5ccc(C)cc5)c4n3)c2)cc(OC)c1OC ; COc1cc(-n2cnc(Nc3nc(N4CCC[C@H]4CO)nc4ccn(S(=O)(=O)c5ccc(C)cc5)c34)c2)cc(OC)c1OC] 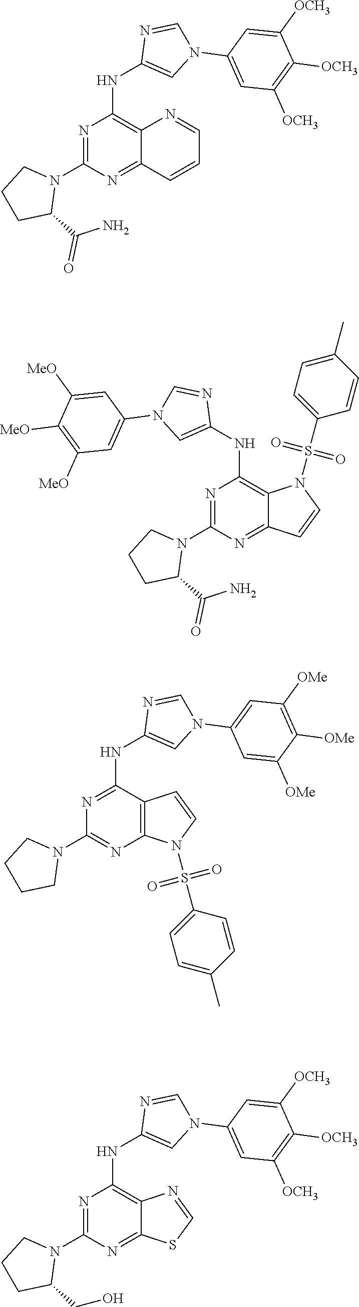 [SMILES: COc1cc(-n2cnc(Nc3nc(N4CCC[C@H]4C(N)=O)nc4cccnc34)c2)cc(OC)c1OC ; COc1cc(-n2cnc(Nc3nc(N4CCC[C@H]4C(N)=O)nc4ccn(S(=O)(=O)c5ccc(C)cc5)c34)c2)cc(OC)c1OC ; COc1cc(-n2cnc(Nc3nc(N4CCCC4)nc4c3ccn4S(=O)(=O)c3ccc(C)cc3)c2)cc(OC)c1OC ; COc1cc(-n2cnc(Nc3nc(N4CCC[C@H]4CO)nc4scnc34)c2)cc(OC)c1OC] 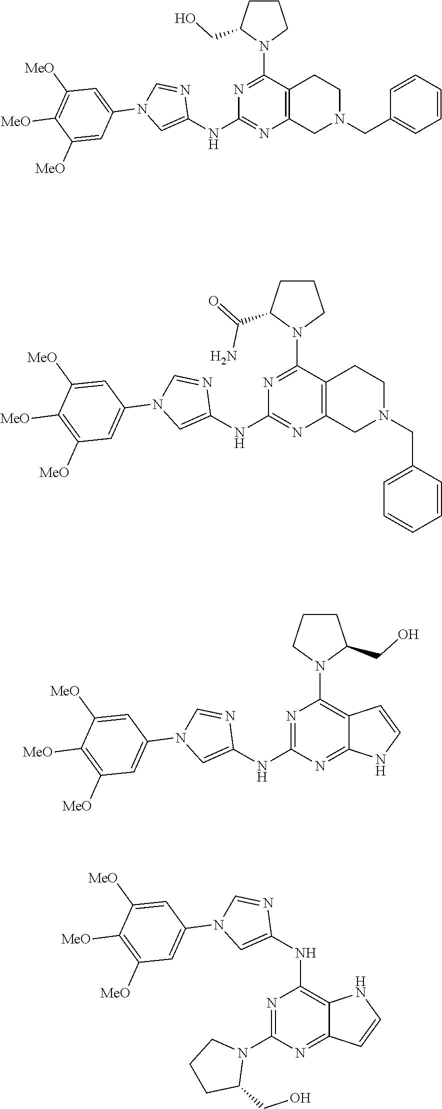 [SMILES: COc1cc(-n2cnc(Nc3nc4c(c(N5CCC[C@H]5CO)n3)CCN(Cc3ccccc3)C4)c2)cc(OC)c1OC ; COc1cc(-n2cnc(Nc3nc4c(c(N5CCC[C@H]5C(N)=O)n3)CCN(Cc3ccccc3)C4)c2)cc(OC)c1OC ; COc1cc(-n2cnc(Nc3nc(N4CCC[C@H]4CO)c4cc[nH]c4n3)c2)cc(OC)c1OC ; COc1cc(-n2cnc(Nc3nc(N4CCC[C@H]4CO)nc4cc[nH]c34)c2)cc(OC)c1OC] 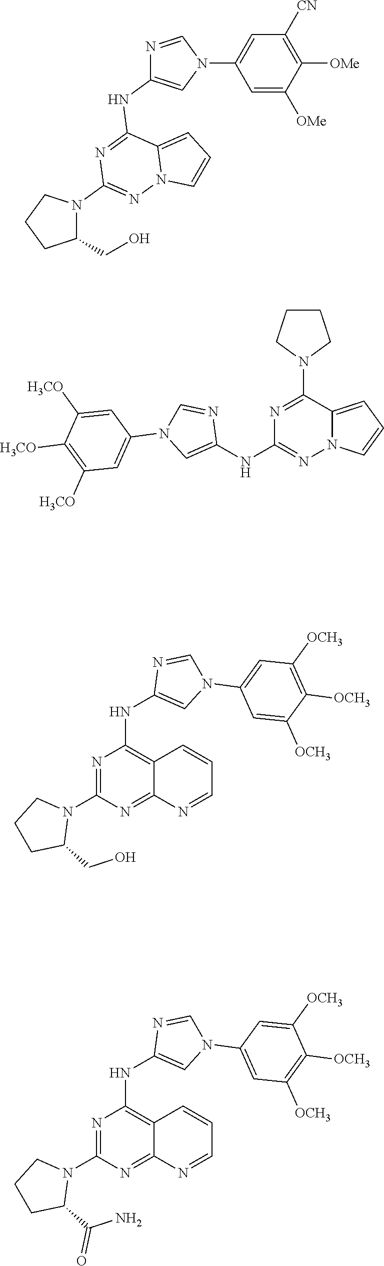 [SMILES: COc1cc(-n2cnc(Nc3nc(N4CCC[C@H]4CO)nn4cccc34)c2)cc(C#N)c1OC ; COc1cc(-n2cnc(Nc3nc(N4CCCC4)c4cccn4n3)c2)cc(OC)c1OC ; COc1cc(-n2cnc(Nc3nc(N4CCC[C@H]4CO)nc4ncccc34)c2)cc(OC)c1OC ; COc1cc(-n2cnc(Nc3nc(N4CCC[C@H]4C(N)=O)nc4ncccc34)c2)cc(OC)c1OC] 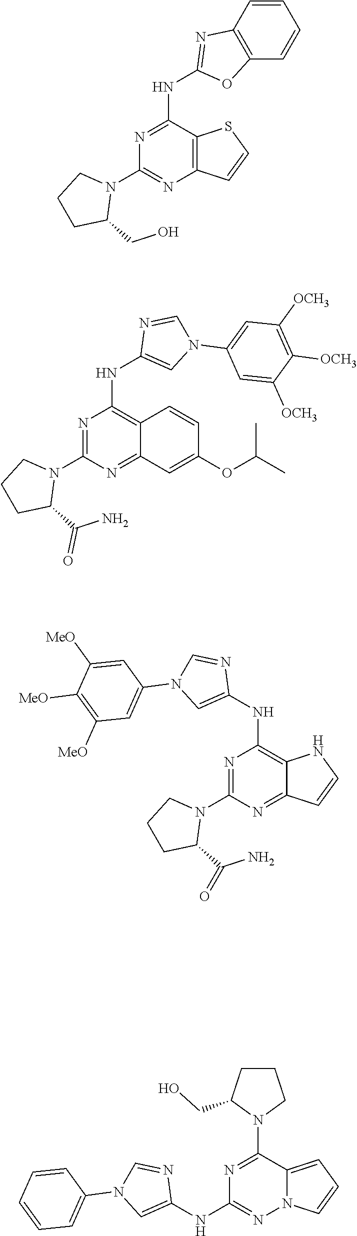 [SMILES: OC[C@@H]1CCCN1c1nc(Nc2nc3ccccc3o2)c2sccc2n1 ; COc1cc(-n2cnc(Nc3nc(N4CCC[C@H]4C(N)=O)nc4cc(OC(C)C)ccc34)c2)cc(OC)c1OC ; COc1cc(-n2cnc(Nc3nc(N4CCC[C@H]4C(N)=O)nc4cc[nH]c34)c2)cc(OC)c1OC ; OC[C@@H]1CCCN1c1nc(Nc2cn(-c3ccccc3)cn2)nn2cccc12] 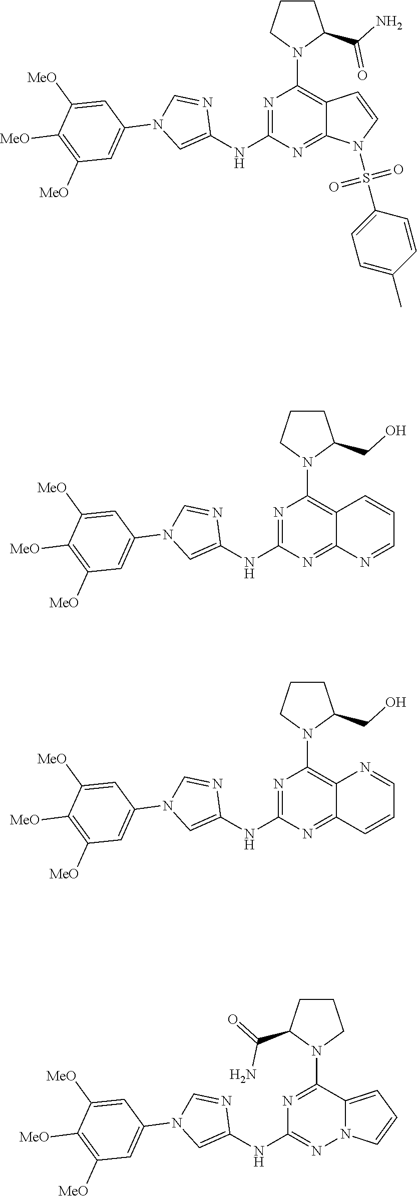 [SMILES: COc1cc(-n2cnc(Nc3nc(N4CCC[C@H]4C(N)=O)c4ccn(S(=O)(=O)c5ccc(C)cc5)c4n3)c2)cc(OC)c1OC ; COc1cc(-n2cnc(Nc3nc(N4CCC[C@H]4CO)c4cccnc4n3)c2)cc(OC)c1OC ; COc1cc(-n2cnc(Nc3nc(N4CCC[C@H]4CO)c4ncccc4n3)c2)cc(OC)c1OC ; COc1cc(-n2cnc(Nc3nc(N4CCC[C@@H]4C(N)=O)c4cccn4n3)c2)cc(OC)c1OC] 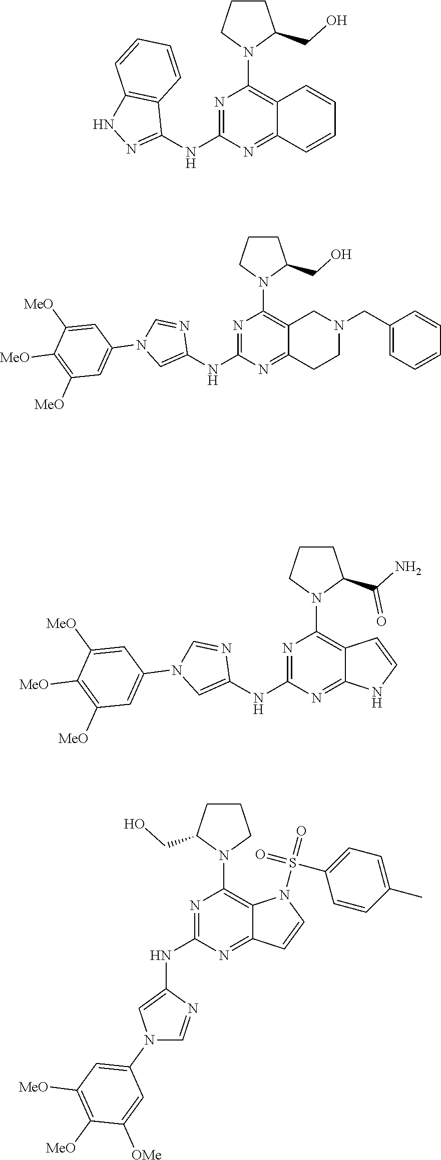 [SMILES: OC[C@@H]1CCCN1c1nc(Nc2n[nH]c3ccccc23)nc2ccccc12 ; COc1cc(-n2cnc(Nc3nc4c(c(N5CCC[C@H]5CO)n3)CN(Cc3ccccc3)CC4)c2)cc(OC)c1OC ; COc1cc(-n2cnc(Nc3nc(N4CCC[C@H]4C(N)=O)c4cc[nH]c4n3)c2)cc(OC)c1OC ; COc1cc(-n2cnc(Nc3nc(N4CCC[C@H]4CO)c4c(ccn4S(=O)(=O)c4ccc(C)cc4)n3)c2)cc(OC)c1OC] 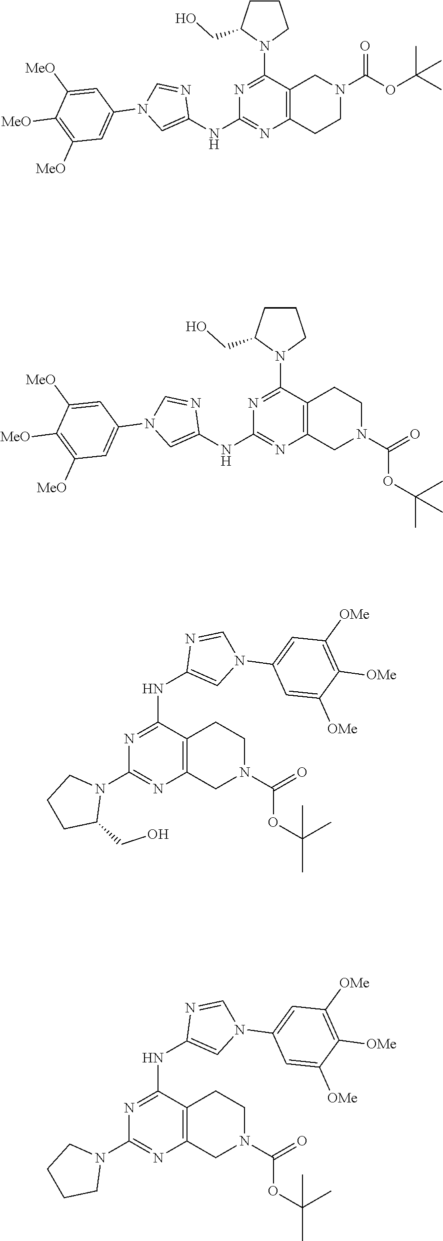 [SMILES: COc1cc(-n2cnc(Nc3nc4c(c(N5CCC[C@H]5CO)n3)CN(C(=O)OC(C)(C)C)CC4)c2)cc(OC)c1OC ; COc1cc(-n2cnc(Nc3nc4c(c(N5CCC[C@H]5CO)n3)CCN(C(=O)OC(C)(C)C)C4)c2)cc(OC)c1OC ; COc1cc(-n2cnc(Nc3nc(N4CCC[C@H]4CO)nc4c3CCN(C(=O)OC(C)(C)C)C4)c2)cc(OC)c1OC ; COc1cc(-n2cnc(Nc3nc(N4CCCC4)nc4c3CCN(C(=O)OC(C)(C)C)C4)c2)cc(OC)c1OC] 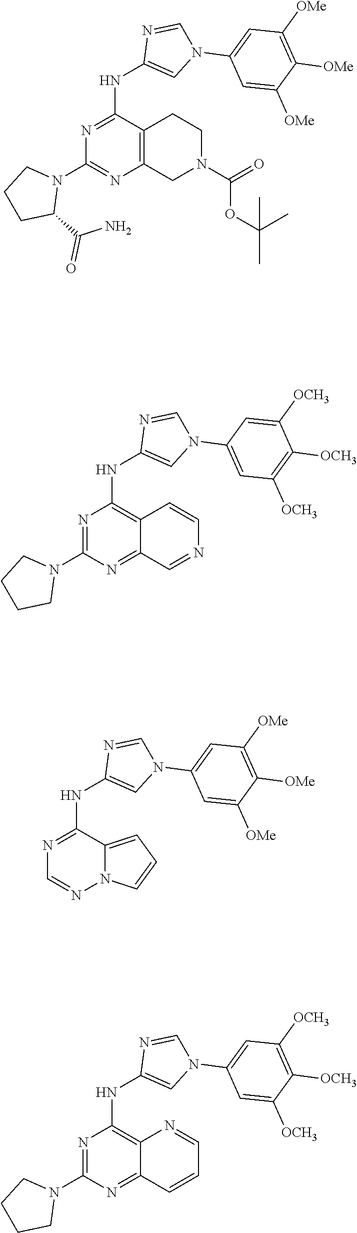 [SMILES: COc1cc(-n2cnc(Nc3nc(N4CCC[C@H]4C(N)=O)nc4c3CCN(C(=O)OC(C)(C)C)C4)c2)cc(OC)c1OC ; COc1cc(-n2cnc(Nc3nc(N4CCCC4)nc4cnccc34)c2)cc(OC)c1OC ; COc1cc(-n2cnc(Nc3ncnn4cccc34)c2)cc(OC)c1OC ; COc1cc(-n2cnc(Nc3nc(N4CCCC4)nc4cccnc34)c2)cc(OC)c1OC] 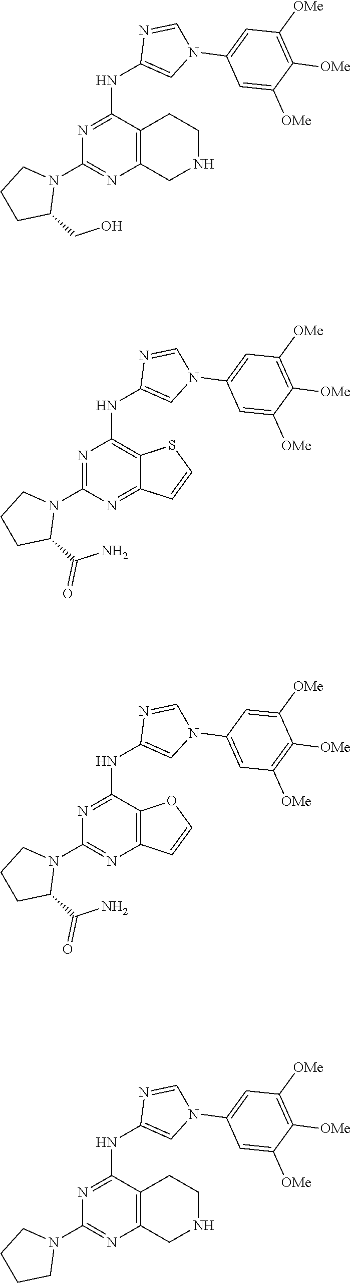 [SMILES: COc1cc(-n2cnc(Nc3nc(N4CCC[C@H]4CO)nc4c3CCNC4)c2)cc(OC)c1OC ; COc1cc(-n2cnc(Nc3nc(N4CCC[C@H]4C(N)=O)nc4ccsc34)c2)cc(OC)c1OC ; COc1cc(-n2cnc(Nc3nc(N4CCC[C@H]4C(N)=O)nc4ccoc34)c2)cc(OC)c1OC ; COc1cc(-n2cnc(Nc3nc(N4CCCC4)nc4c3CCNC4)c2)cc(OC)c1OC] 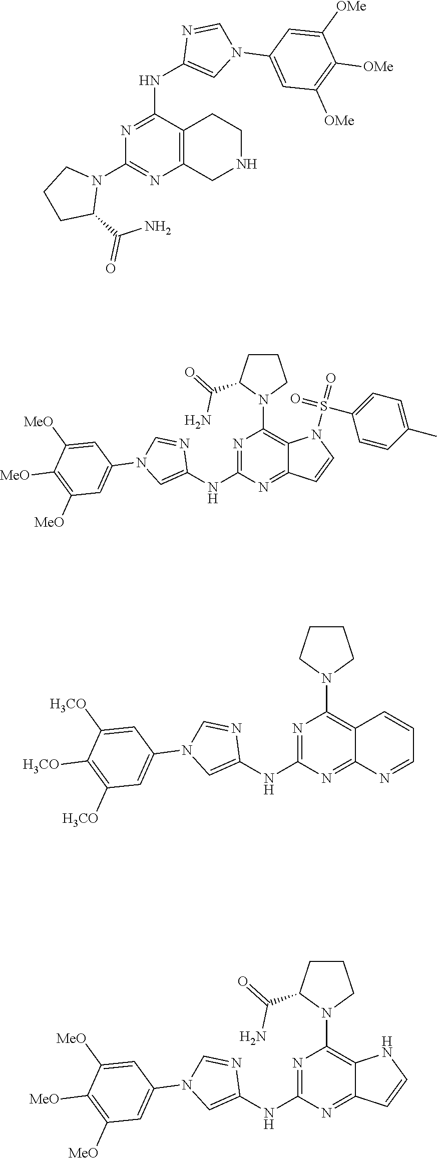 [SMILES: COc1cc(-n2cnc(Nc3nc(N4CCC[C@H]4C(N)=O)nc4c3CCNC4)c2)cc(OC)c1OC ; COc1cc(-n2cnc(Nc3nc(N4CCC[C@H]4C(N)=O)c4c(ccn4S(=O)(=O)c4ccc(C)cc4)n3)c2)cc(OC)c1OC ; COc1cc(-n2cnc(Nc3nc(N4CCCC4)c4cccnc4n3)c2)cc(OC)c1OC ; COc1cc(-n2cnc(Nc3nc(N4CCC[C@H]4C(N)=O)c4[nH]ccc4n3)c2)cc(OC)c1OC] 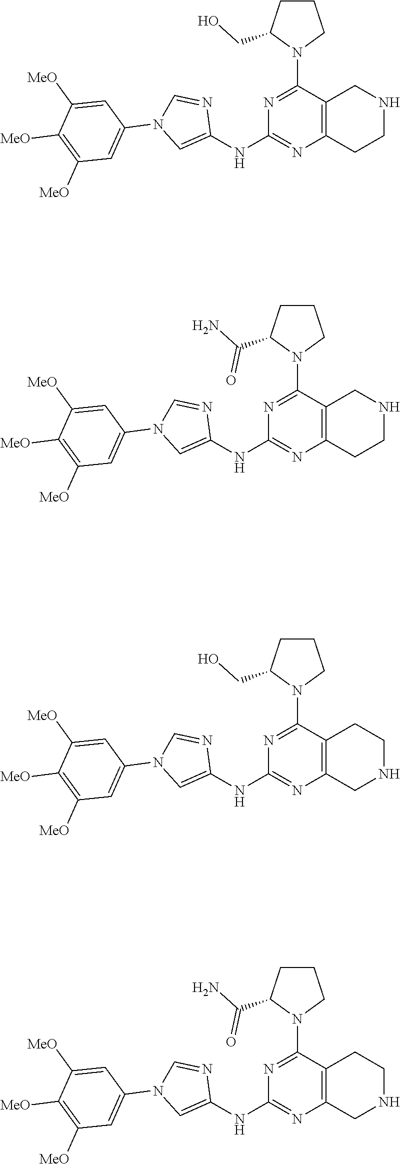 [SMILES: COc1cc(-n2cnc(Nc3nc4c(c(N5CCC[C@H]5CO)n3)CNCC4)c2)cc(OC)c1OC ; COc1cc(-n2cnc(Nc3nc4c(c(N5CCC[C@H]5C(N)=O)n3)CNCC4)c2)cc(OC)c1OC ; COc1cc(-n2cnc(Nc3nc4c(c(N5CCC[C@H]5CO)n3)CCNC4)c2)cc(OC)c1OC ; COc1cc(-n2cnc(Nc3nc4c(c(N5CCC[C@H]5C(N)=O)n3)CCNC4)c2)cc(OC)c1OC] 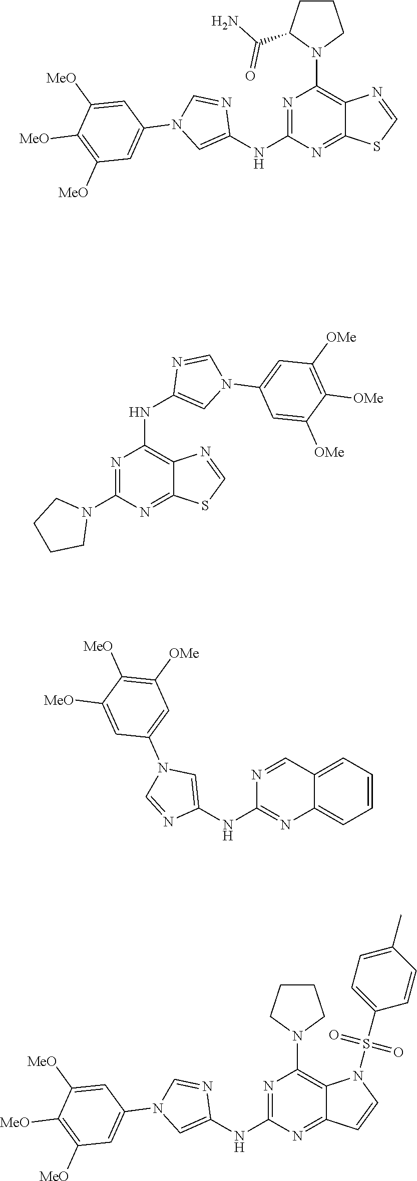 [SMILES: COc1cc(-n2cnc(Nc3nc(N4CCC[C@H]4C(N)=O)c4ncsc4n3)c2)cc(OC)c1OC ; COc1cc(-n2cnc(Nc3nc(N4CCCC4)nc4scnc34)c2)cc(OC)c1OC ; COc1cc(-n2cnc(Nc3ncc4ccccc4n3)c2)cc(OC)c1OC ; COc1cc(-n2cnc(Nc3nc(N4CCCC4)c4c(ccn4S(=O)(=O)c4ccc(C)cc4)n3)c2)cc(OC)c1OC] 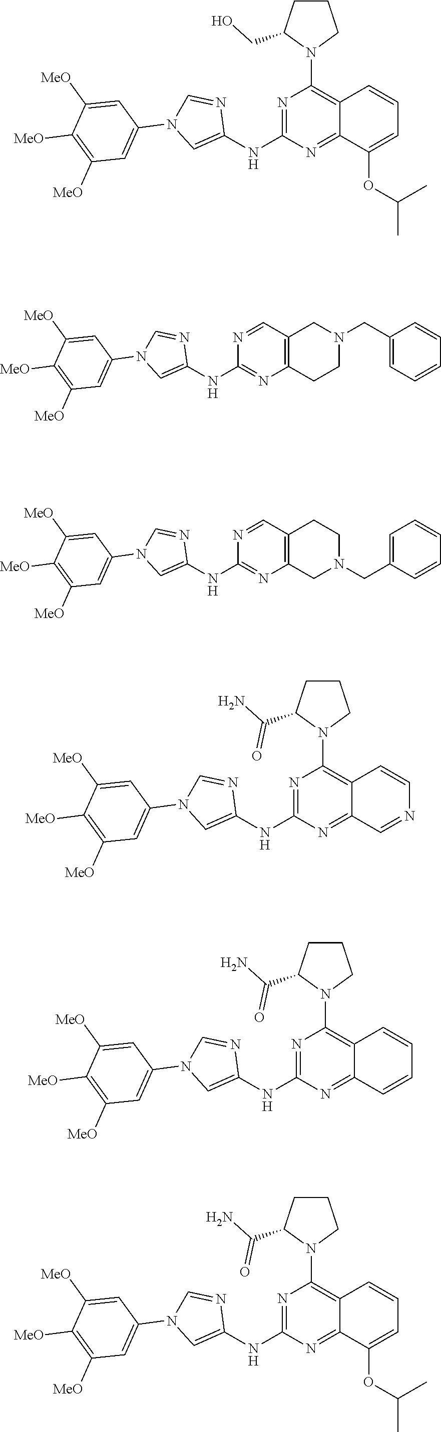 [SMILES: COc1cc(-n2cnc(Nc3nc(N4CCC[C@H]4CO)c4cccc(OC(C)C)c4n3)c2)cc(OC)c1OC ; COc1cc(-n2cnc(Nc3ncc4c(n3)CCN(Cc3ccccc3)C4)c2)cc(OC)c1OC ; COc1cc(-n2cnc(Nc3ncc4c(n3)CN(Cc3ccccc3)CC4)c2)cc(OC)c1OC ; COc1cc(-n2cnc(Nc3nc(N4CCC[C@H]4C(N)=O)c4ccncc4n3)c2)cc(OC)c1OC ; COc1cc(-n2cnc(Nc3nc(N4CCC[C@H]4C(N)=O)c4ccccc4n3)c2)cc(OC)c1OC ; COc1cc(-n2cnc(Nc3nc(N4CCC[C@H]4C(N)=O)c4cccc(OC(C)C)c4n3)c2)cc(OC)c1OC] 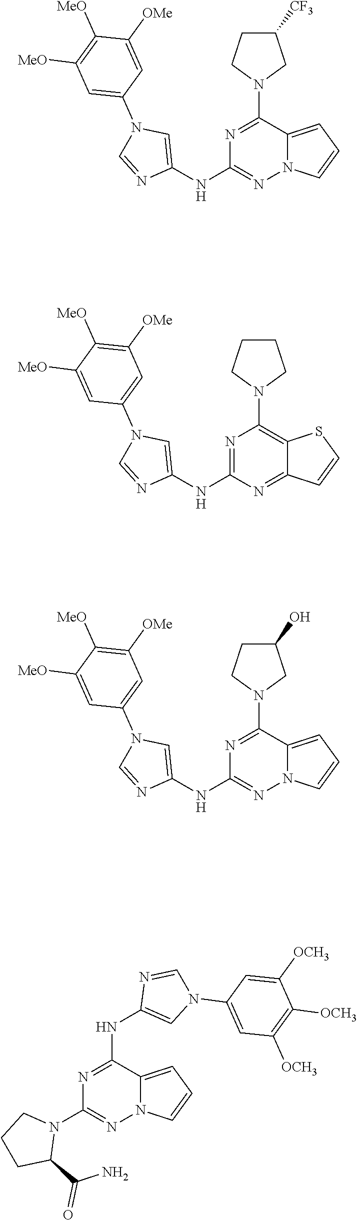 [SMILES: COc1cc(-n2cnc(Nc3nc(N4CC[C@H](C(F)(F)F)C4)c4cccn4n3)c2)cc(OC)c1OC ; COc1cc(-n2cnc(Nc3nc(N4CCCC4)c4sccc4n3)c2)cc(OC)c1OC ; COc1cc(-n2cnc(Nc3nc(N4CC[C@@H](O)C4)c4cccn4n3)c2)cc(OC)c1OC ; COc1cc(-n2cnc(Nc3nc(N4CCC[C@@H]4C(N)=O)nn4cccc34)c2)cc(OC)c1OC] 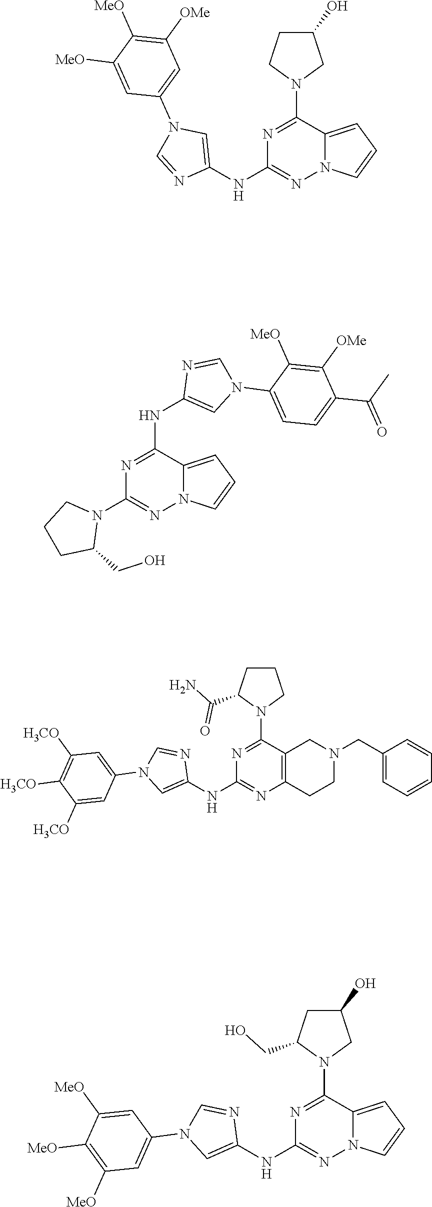 [SMILES: COc1cc(-n2cnc(Nc3nc(N4CC[C@H](O)C4)c4cccn4n3)c2)cc(OC)c1OC ; COc1c(C(C)=O)ccc(-n2cnc(Nc3nc(N4CCC[C@H]4CO)nn4cccc34)c2)c1OC ; COc1cc(-n2cnc(Nc3nc4c(c(N5CCC[C@H]5C(N)=O)n3)CN(Cc3ccccc3)CC4)c2)cc(OC)c1OC ; COc1cc(-n2cnc(Nc3nc(N4C[C@H](O)C[C@H]4CO)c4cccn4n3)c2)cc(OC)c1OC] 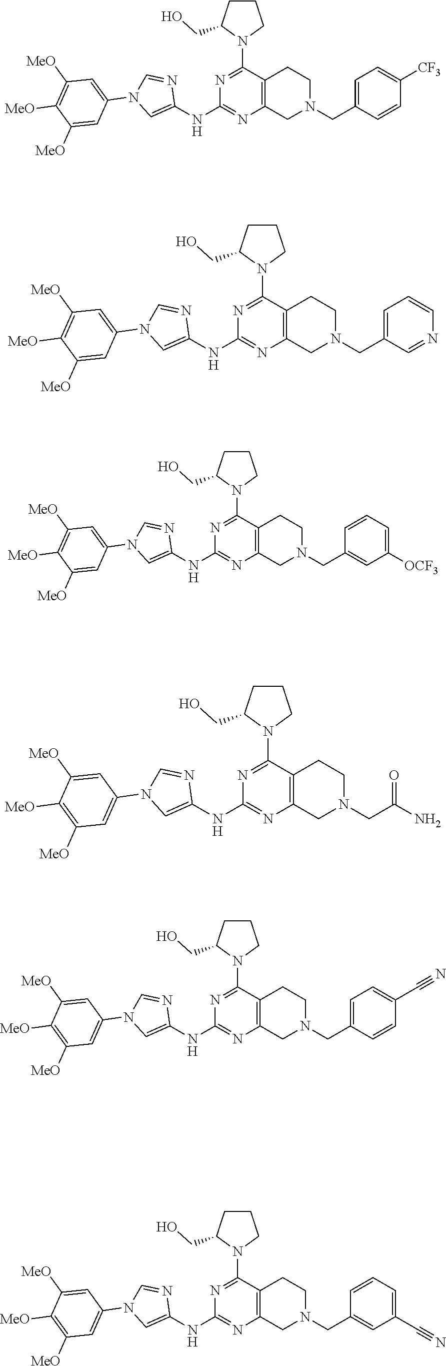 [SMILES: COc1cc(-n2cnc(Nc3nc4c(c(N5CCC[C@H]5CO)n3)CCN(Cc3ccc(C(F)(F)F)cc3)C4)c2)cc(OC)c1OC ; COc1cc(-n2cnc(Nc3nc4c(c(N5CCC[C@H]5CO)n3)CCN(Cc3cccnc3)C4)c2)cc(OC)c1OC ; COc1cc(-n2cnc(Nc3nc4c(c(N5CCC[C@H]5CO)n3)CCN(Cc3cccc(OC(F)(F)F)c3)C4)c2)cc(OC)c1OC ; COc1cc(-n2cnc(Nc3nc4c(c(N5CCC[C@H]5CO)n3)CCN(CC(N)=O)C4)c2)cc(OC)c1OC ; COc1cc(-n2cnc(Nc3nc4c(c(N5CCC[C@H]5CO)n3)CCN(Cc3ccc(C#N)cc3)C4)c2)cc(OC)c1OC ; COc1cc(-n2cnc(Nc3nc4c(c(N5CCC[C@H]5CO)n3)CCN(Cc3cccc(C#N)c3)C4)c2)cc(OC)c1OC] 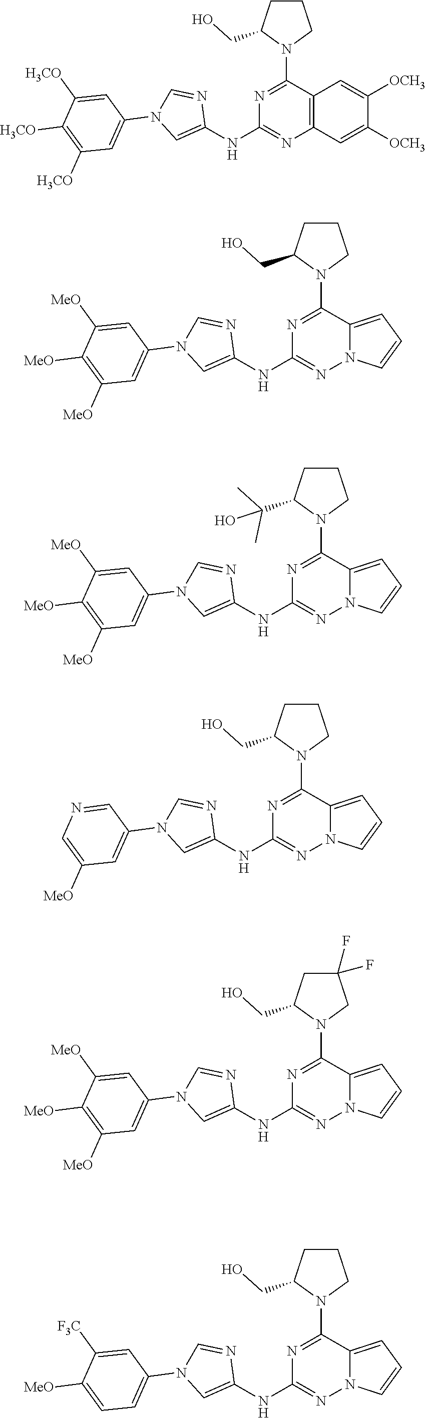 [SMILES: COc1cc2nc(Nc3cn(-c4cc(OC)c(OC)c(OC)c4)cn3)nc(N3CCC[C@H]3CO)c2cc1OC ; COc1cc(-n2cnc(Nc3nc(N4CCC[C@@H]4CO)c4cccn4n3)c2)cc(OC)c1OC ; COc1cc(-n2cnc(Nc3nc(N4CCC[C@H]4C(C)(C)O)c4cccn4n3)c2)cc(OC)c1OC ; COc1cncc(-n2cnc(Nc3nc(N4CCC[C@H]4CO)c4cccn4n3)c2)c1 ; COc1cc(-n2cnc(Nc3nc(N4CC(F)(F)C[C@H]4CO)c4cccn4n3)c2)cc(OC)c1OC ; COc1ccc(-n2cnc(Nc3nc(N4CCC[C@H]4CO)c4cccn4n3)c2)cc1C(F)(F)F] 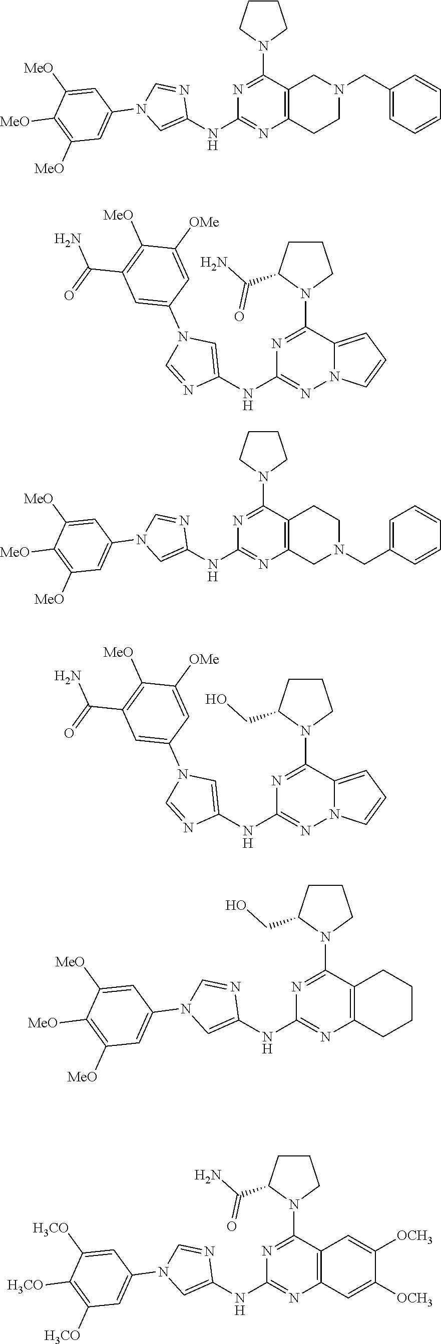 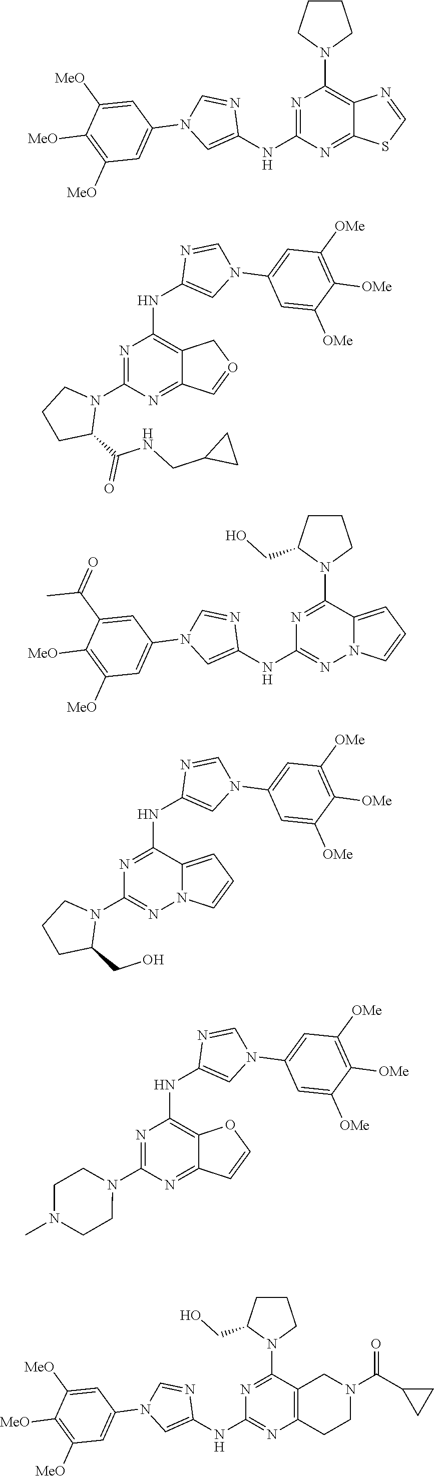 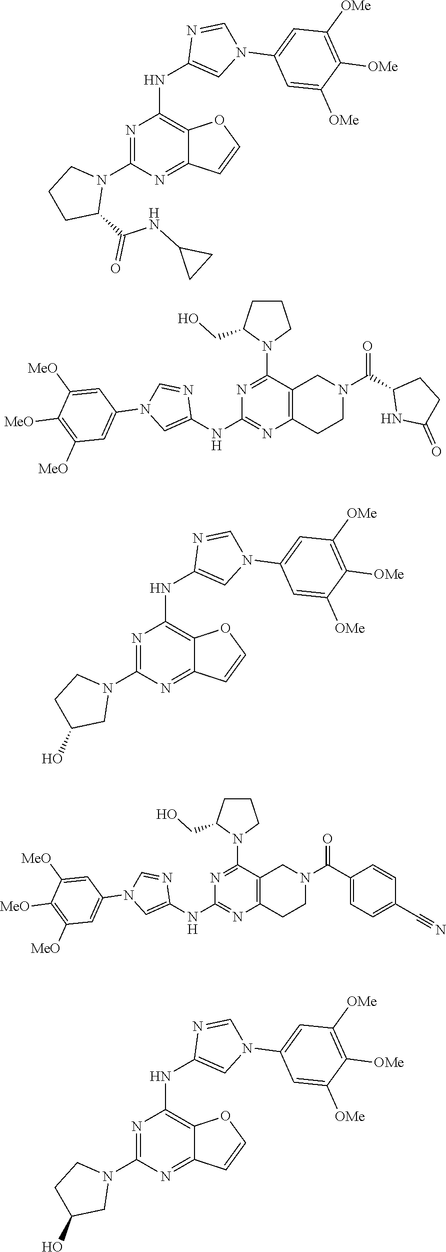 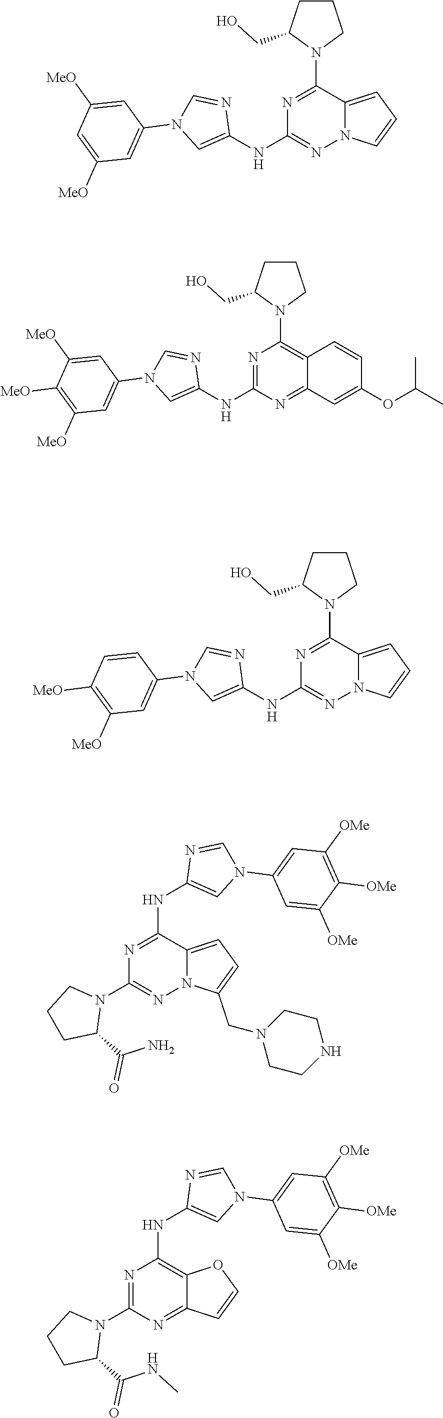 [SMILES: COc1cc(OC)cc(-n2cnc(Nc3nc(N4CCC[C@H]4CO)c4cccn4n3)c2)c1 ; COc1cc(-n2cnc(Nc3nc(N4CCC[C@H]4CO)c4ccc(OC(C)C)cc4n3)c2)cc(OC)c1OC ; COc1ccc(-n2cnc(Nc3nc(N4CCC[C@H]4CO)c4cccn4n3)c2)cc1OC ; COc1cc(-n2cnc(Nc3nc(N4CCC[C@H]4C(N)=O)nn4c(CN5CCNCC5)ccc34)c2)cc(OC)c1OC ; CNC(=O)[C@@H]1CCCN1c1nc(Nc2cn(-c3cc(OC)c(OC)c(OC)c3)cn2)c2occc2n1] 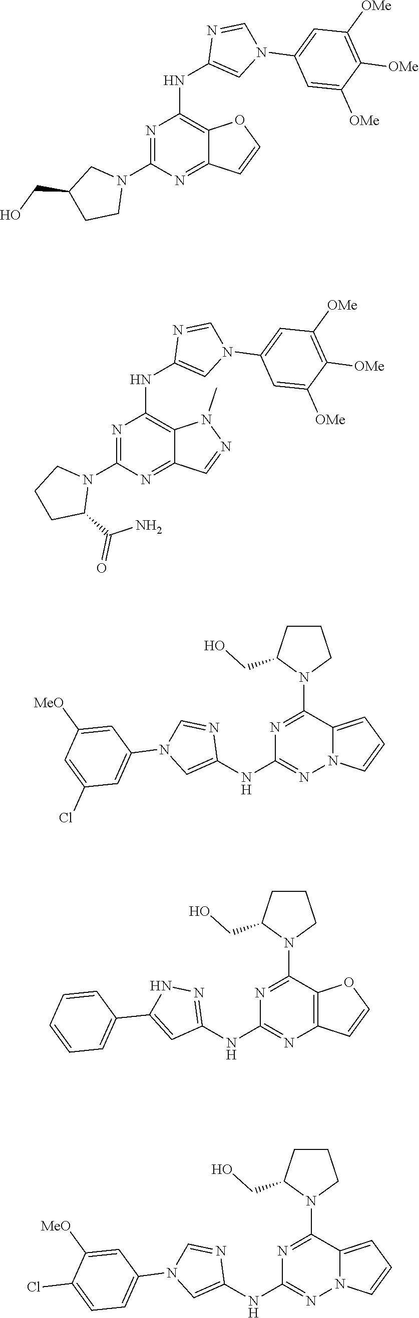 [SMILES: COc1cc(-n2cnc(Nc3nc(N4CC[C@@H](CO)C4)nc4ccoc34)c2)cc(OC)c1OC ; COc1cc(-n2cnc(Nc3nc(N4CCC[C@H]4C(N)=O)nc4cnn(C)c34)c2)cc(OC)c1OC ; COc1cc(Cl)cc(-n2cnc(Nc3nc(N4CCC[C@H]4CO)c4cccn4n3)c2)c1 ; OC[C@@H]1CCCN1c1nc(Nc2cc(-c3ccccc3)[nH]n2)nc2ccoc12 ; COc1cc(-n2cnc(Nc3nc(N4CCC[C@H]4CO)c4cccn4n3)c2)ccc1Cl] 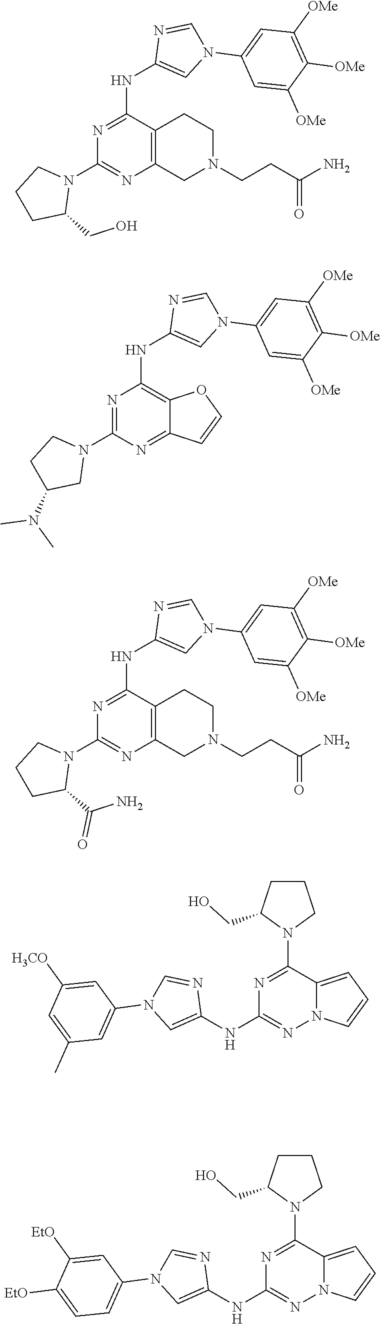 [SMILES: COc1cc(-n2cnc(Nc3nc(N4CCC[C@H]4CO)nc4c3CCN(CCC(N)=O)C4)c2)cc(OC)c1OC ; COc1cc(-n2cnc(Nc3nc(N4CC[C@@H](N(C)C)C4)nc4ccoc34)c2)cc(OC)c1OC ; COc1cc(-n2cnc(Nc3nc(N4CCC[C@H]4C(N)=O)nc4c3CCN(CCC(N)=O)C4)c2)cc(OC)c1OC ; COc1cc(C)cc(-n2cnc(Nc3nc(N4CCC[C@H]4CO)c4cccn4n3)c2)c1 ; CCOc1ccc(-n2cnc(Nc3nc(N4CCC[C@H]4CO)c4cccn4n3)c2)cc1OCC] 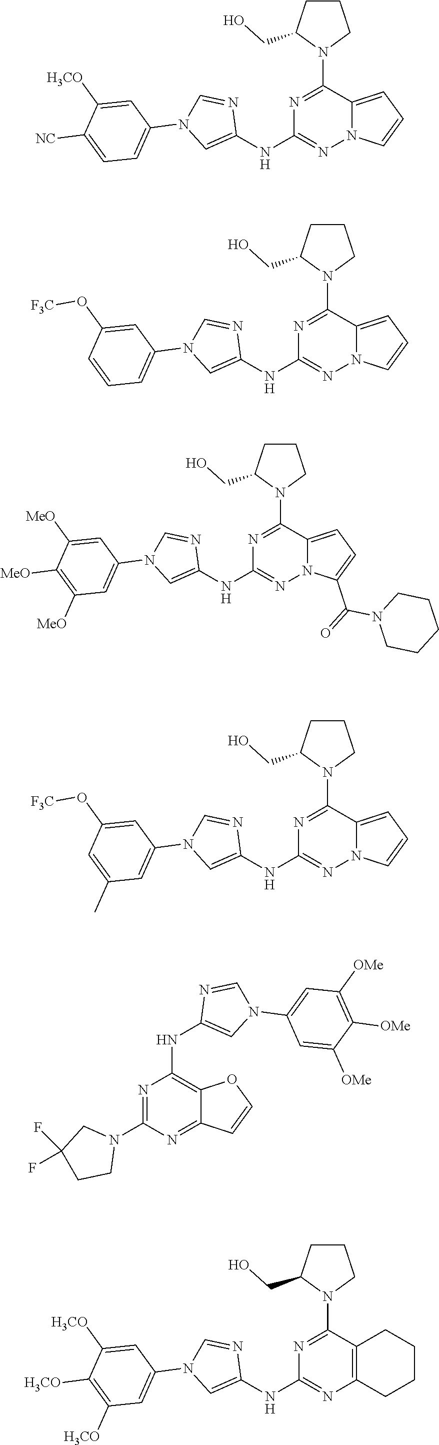 [SMILES: COc1cc(-n2cnc(Nc3nc(N4CCC[C@H]4CO)c4cccn4n3)c2)ccc1C#N ; OC[C@@H]1CCCN1c1nc(Nc2cn(-c3cccc(OC(F)(F)F)c3)cn2)nn2cccc12 ; COc1cc(-n2cnc(Nc3nc(N4CCC[C@H]4CO)c4ccc(C(=O)N5CCCCC5)n4n3)c2)cc(OC)c1OC ; Cc1cc(OC(F)(F)F)cc(-n2cnc(Nc3nc(N4CCC[C@H]4CO)c4cccn4n3)c2)c1 ; COc1cc(-n2cnc(Nc3nc(N4CCC(F)(F)C4)nc4ccoc34)c2)cc(OC)c1OC ; COc1cc(-n2cnc(Nc3nc4c(c(N5CCC[C@@H]5CO)n3)CCCC4)c2)cc(OC)c1OC] 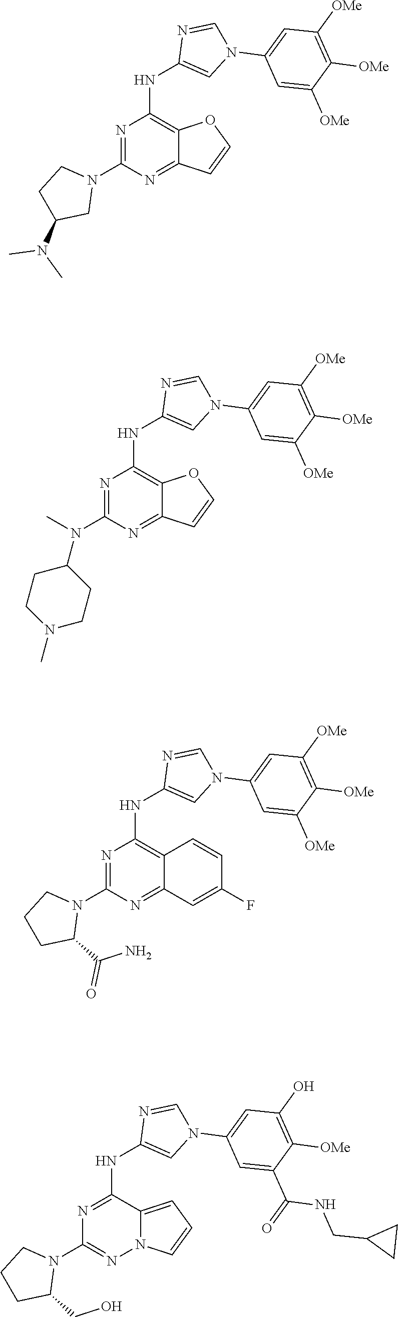 [SMILES: COc1cc(-n2cnc(Nc3nc(N4CC[C@H](N(C)C)C4)nc4ccoc34)c2)cc(OC)c1OC ; COc1cc(-n2cnc(Nc3nc(N(C)C4CCN(C)CC4)nc4ccoc34)c2)cc(OC)c1OC ; COc1cc(-n2cnc(Nc3nc(N4CCC[C@H]4C(N)=O)nc4cc(F)ccc34)c2)cc(OC)c1OC ; COc1c(O)cc(-n2cnc(Nc3nc(N4CCC[C@H]4CO)nn4cccc34)c2)cc1C(=O)NCC1CC1] 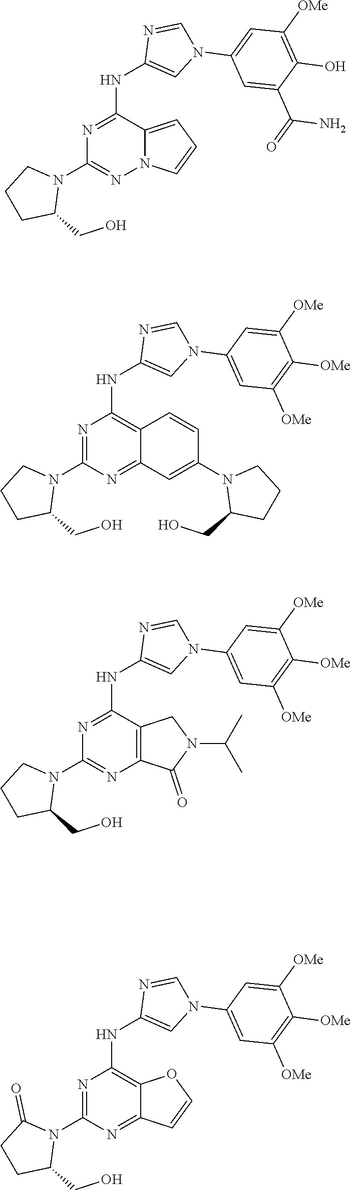 [SMILES: COc1cc(-n2cnc(Nc3nc(N4CCC[C@H]4CO)nn4cccc34)c2)cc(C(N)=O)c1O ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4CO)nc4c3CN(C(C)C)C4=O)c2)cc(OC)c1OC ; COc1cc(-n2cnc(Nc3nc(N4C(=O)CC[C@H]4CO)nc4ccoc34)c2)cc(OC)c1OC] 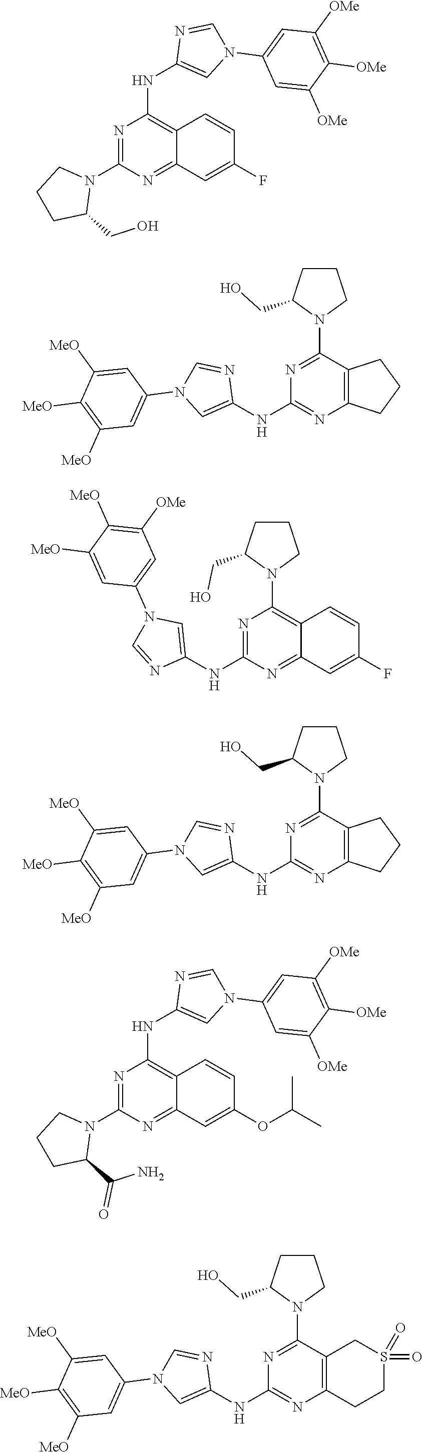 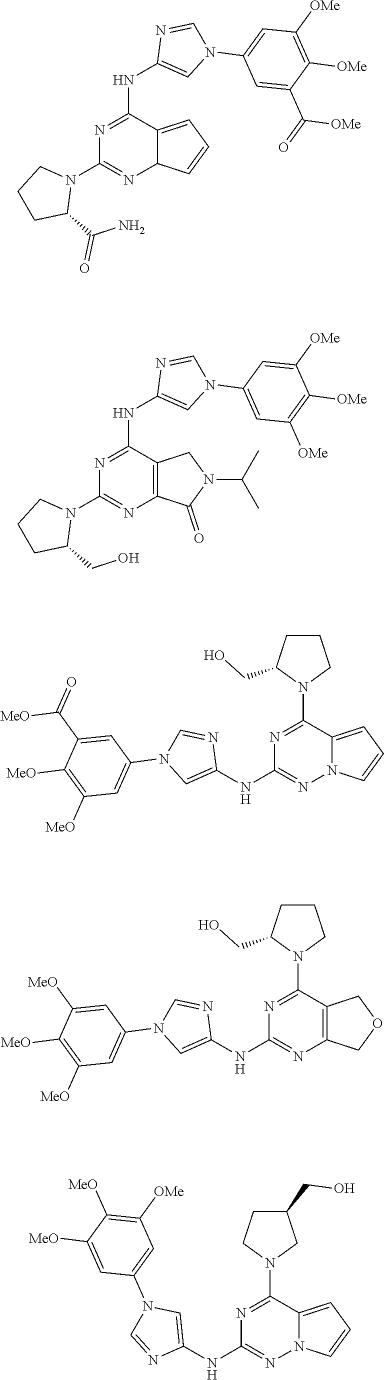 [SMILES: COC(=O)c1cc(-n2cnc(NC3=NC(N4CCC[C@H]4C(N)=O)=NC4C=CC=C34)c2)cc(OC)c1OC ; COc1cc(-n2cnc(Nc3nc(N4CCC[C@H]4CO)nc4c3CN(C(C)C)C4=O)c2)cc(OC)c1OC ; COC(=O)c1cc(-n2cnc(Nc3nc(N4CCC[C@H]4CO)c4cccn4n3)c2)cc(OC)c1OC ; COc1cc(-n2cnc(Nc3nc4c(c(N5CCC[C@H]5CO)n3)COC4)c2)cc(OC)c1OC ; COc1cc(-n2cnc(Nc3nc(N4CC[C@@H](CO)C4)c4cccn4n3)c2)cc(OC)c1OC] 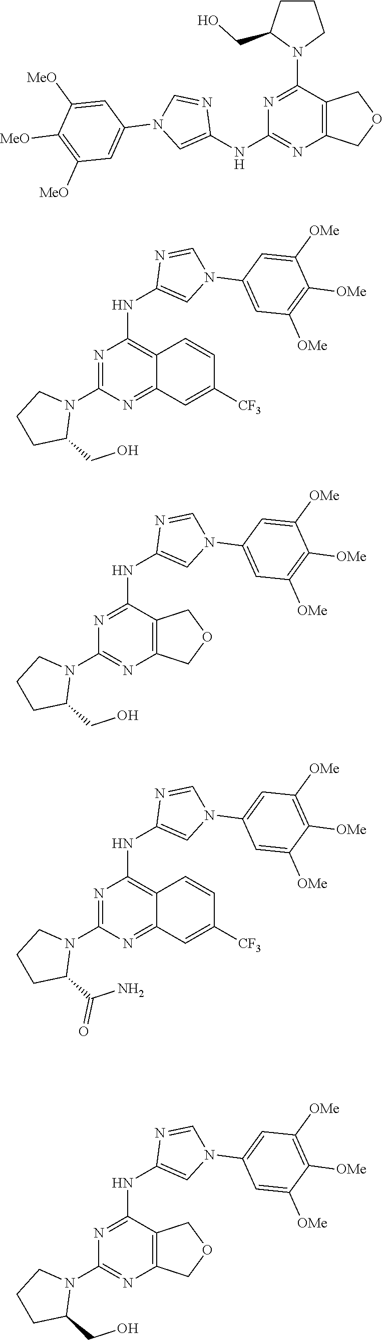 [SMILES: COc1cc(-n2cnc(Nc3nc4c(c(N5CCC[C@@H]5CO)n3)COC4)c2)cc(OC)c1OC ; COc1cc(-n2cnc(Nc3nc(N4CCC[C@H]4CO)nc4cc(C(F)(F)F)ccc34)c2)cc(OC)c1OC ; COc1cc(-n2cnc(Nc3nc(N4CCC[C@H]4CO)nc4c3COC4)c2)cc(OC)c1OC ; COc1cc(-n2cnc(Nc3nc(N4CCC[C@H]4C(N)=O)nc4cc(C(F)(F)F)ccc34)c2)cc(OC)c1OC ; COc1cc(-n2cnc(Nc3nc(N4CCC[C@@H]4CO)nc4c3COC4)c2)cc(OC)c1OC] 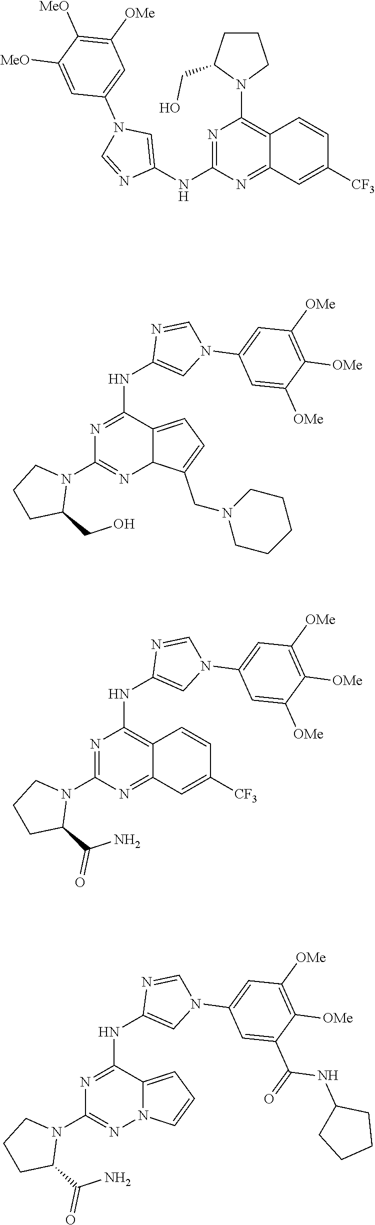 [SMILES: COc1cc(-n2cnc(Nc3nc(N4CCC[C@H]4CO)c4ccc(C(F)(F)F)cc4n3)c2)cc(OC)c1OC ; COc1cc(-n2cnc(NC3=NC(N4CCC[C@@H]4CO)=NC4C(CN5CCCCC5)=CC=C34)c2)cc(OC)c1OC ; COc1cc(-n2cnc(Nc3nc(N4CCC[C@@H]4C(N)=O)nc4cc(C(F)(F)F)ccc34)c2)cc(OC)c1OC ; COc1cc(-n2cnc(Nc3nc(N4CCC[C@H]4C(N)=O)nn4cccc34)c2)cc(C(=O)NC2CCCC2)c1OC] 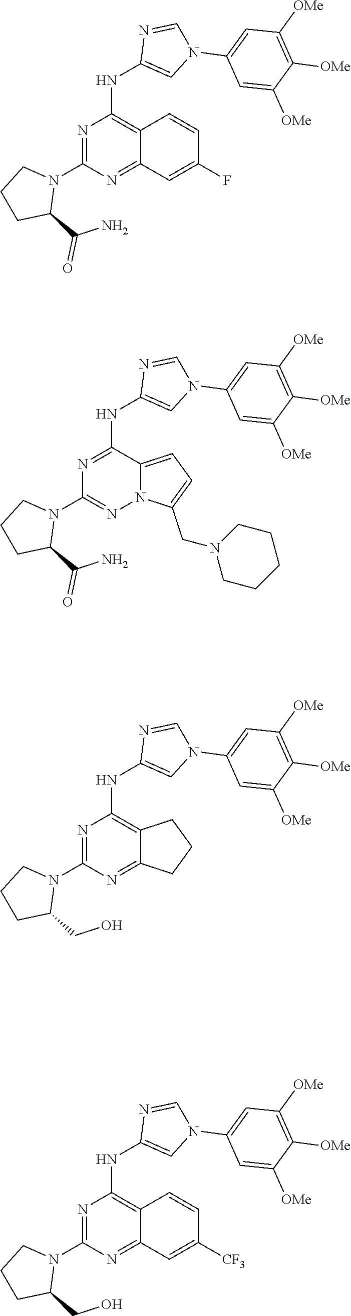 [SMILES: COc1cc(-n2cnc(Nc3nc(N4CCC[C@@H]4C(N)=O)nc4cc(F)ccc34)c2)cc(OC)c1OC ; COc1cc(-n2cnc(Nc3nc(N4CCC[C@@H]4C(N)=O)nn4c(CN5CCCCC5)ccc34)c2)cc(OC)c1OC ; COc1cc(-n2cnc(Nc3nc(N4CCC[C@H]4CO)nc4c3CCC4)c2)cc(OC)c1OC ; COc1cc(-n2cnc(Nc3nc(N4CCC[C@@H]4CO)nc4cc(C(F)(F)F)ccc34)c2)cc(OC)c1OC] 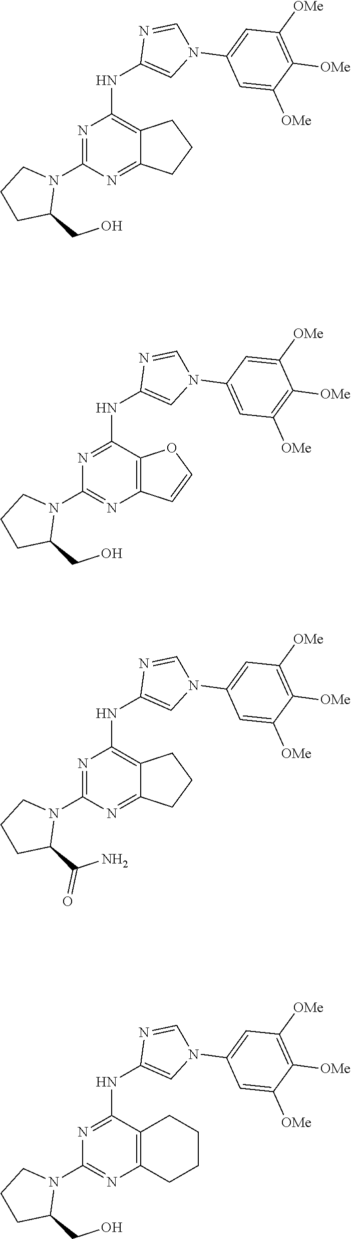 [SMILES: COc1cc(-n2cnc(Nc3nc(N4CCC[C@@H]4CO)nc4c3CCC4)c2)cc(OC)c1OC ; COc1cc(-n2cnc(Nc3nc(N4CCC[C@@H]4CO)nc4ccoc34)c2)cc(OC)c1OC ; COc1cc(-n2cnc(Nc3nc(N4CCC[C@@H]4C(N)=O)nc4c3CCC4)c2)cc(OC)c1OC ; COc1cc(-n2cnc(Nc3nc(N4CCC[C@@H]4CO)nc4c3CCCC4)c2)cc(OC)c1OC] 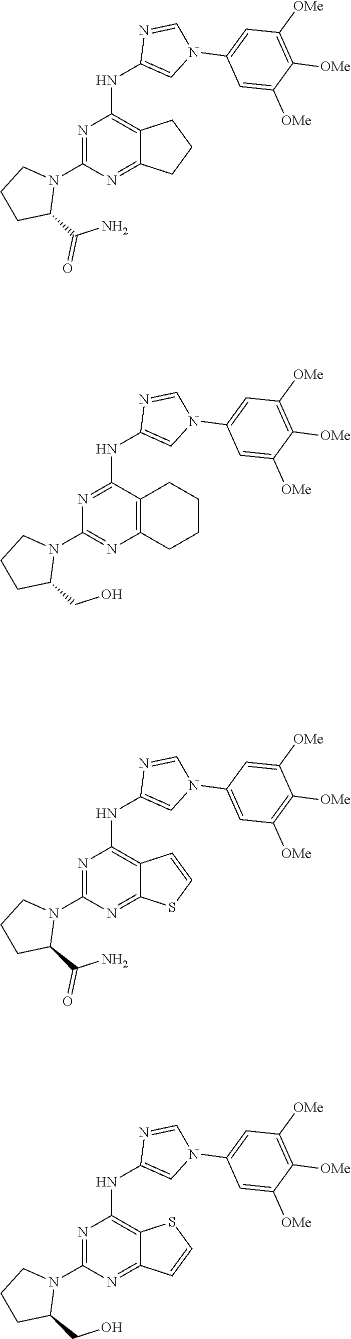 [SMILES: COc1cc(-n2cnc(Nc3nc(N4CCC[C@H]4C(N)=O)nc4c3CCC4)c2)cc(OC)c1OC ; COc1cc(-n2cnc(Nc3nc(N4CCC[C@H]4CO)nc4c3CCCC4)c2)cc(OC)c1OC ; COc1cc(-n2cnc(Nc3nc(N4CCC[C@@H]4C(N)=O)nc4sccc34)c2)cc(OC)c1OC ; COc1cc(-n2cnc(Nc3nc(N4CCC[C@@H]4CO)nc4ccsc34)c2)cc(OC)c1OC] 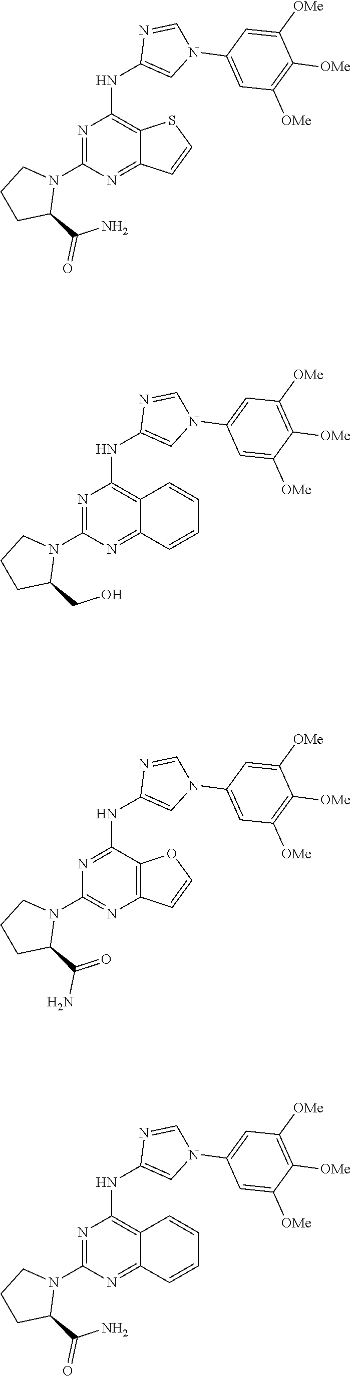 [SMILES: COc1cc(-n2cnc(Nc3nc(N4CCC[C@@H]4C(N)=O)nc4ccsc34)c2)cc(OC)c1OC ; COc1cc(-n2cnc(Nc3nc(N4CCC[C@@H]4CO)nc4ccccc34)c2)cc(OC)c1OC ; COc1cc(-n2cnc(Nc3nc(N4CCC[C@@H]4C(N)=O)nc4ccoc34)c2)cc(OC)c1OC ; COc1cc(-n2cnc(Nc3nc(N4CCC[C@@H]4C(N)=O)nc4ccccc34)c2)cc(OC)c1OC] 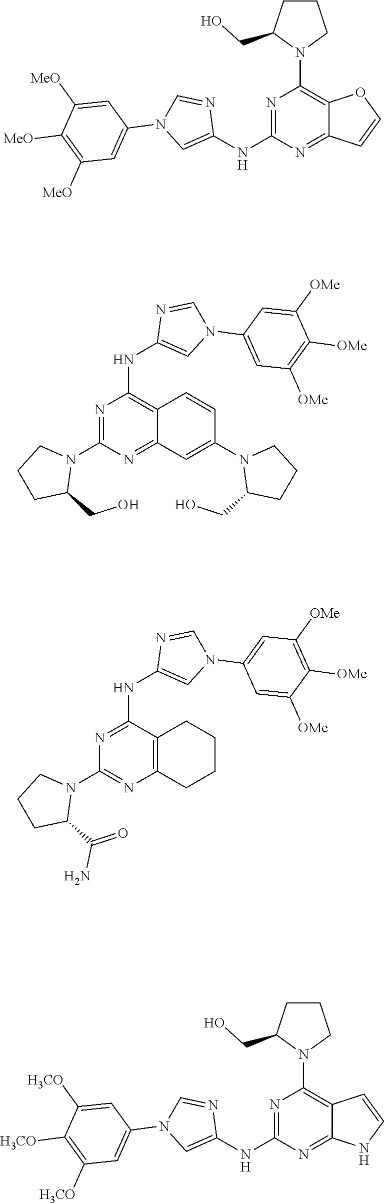 [SMILES: COc1cc(-n2cnc(Nc3nc(N4CCC[C@@H]4CO)c4occc4n3)c2)cc(OC)c1OC ; COc1cc(-n2cnc(Nc3nc(N4CCC[C@@H]4CO)nc4cc(N5CCC[C@@H]5CO)ccc34)c2)cc(OC)c1OC ; COc1cc(-n2cnc(Nc3nc(N4CCC[C@H]4C(N)=O)nc4c3CCCC4)c2)cc(OC)c1OC ; COc1cc(-n2cnc(Nc3nc(N4CCC[C@@H]4CO)c4cc[nH]c4n3)c2)cc(OC)c1OC] 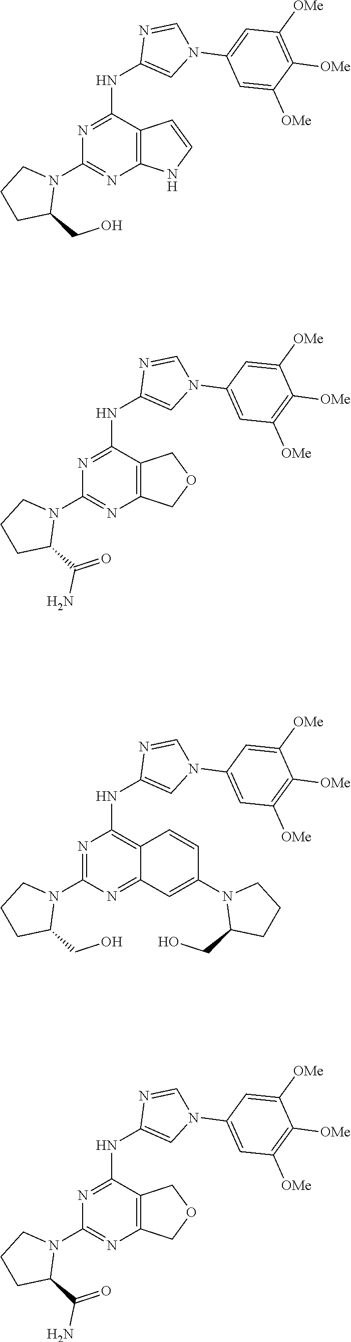 [SMILES: COc1cc(-n2cnc(Nc3nc(N4CCC[C@@H]4CO)nc4[nH]ccc34)c2)cc(OC)c1OC ; COc1cc(-n2cnc(Nc3nc(N4CCC[C@H]4C(N)=O)nc4c3COC4)c2)cc(OC)c1OC ; COc1cc(-n2cnc(Nc3nc(N4CCC[C@H]4CO)nc4cc(N5CCC[C@H]5CO)ccc34)c2)cc(OC)c1OC ; COc1cc(-n2cnc(Nc3nc(N4CCC[C@@H]4C(N)=O)nc4c3COC4)c2)cc(OC)c1OC] 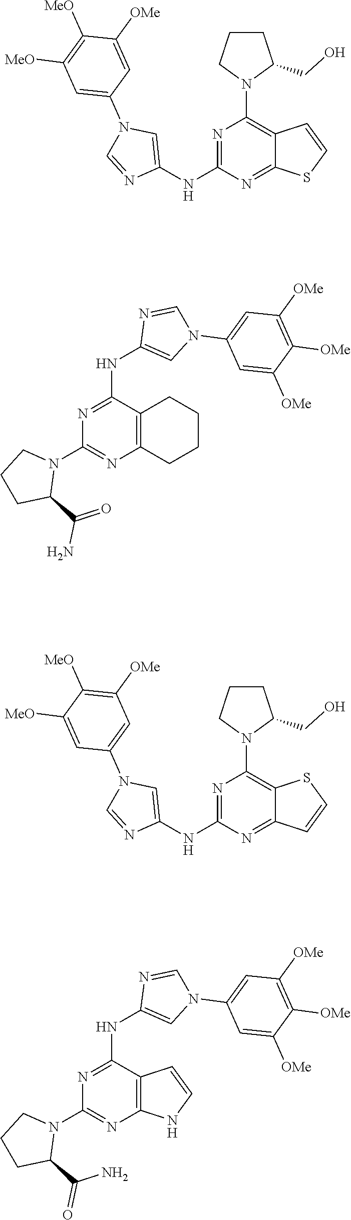 [SMILES: COc1cc(-n2cnc(Nc3nc(N4CCC[C@@H]4CO)c4ccsc4n3)c2)cc(OC)c1OC ; COc1cc(-n2cnc(Nc3nc(N4CCC[C@@H]4C(N)=O)nc4c3CCCC4)c2)cc(OC)c1OC ; COc1cc(-n2cnc(Nc3nc(N4CCC[C@@H]4CO)c4sccc4n3)c2)cc(OC)c1OC ; COc1cc(-n2cnc(Nc3nc(N4CCC[C@@H]4C(N)=O)nc4[nH]ccc34)c2)cc(OC)c1OC] 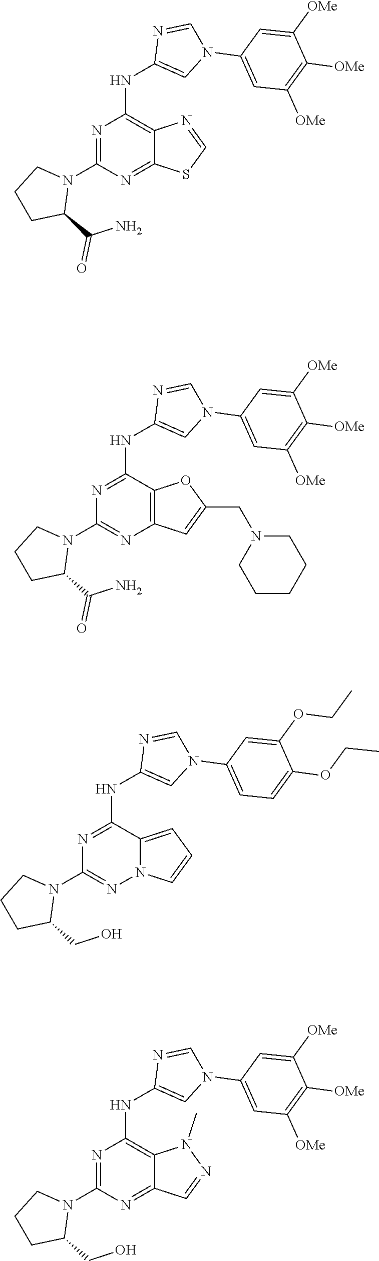 [SMILES: COc1cc(-n2cnc(Nc3nc(N4CCC[C@@H]4C(N)=O)nc4scnc34)c2)cc(OC)c1OC ; COc1cc(-n2cnc(Nc3nc(N4CCC[C@H]4C(N)=O)nc4cc(CN5CCCCC5)oc34)c2)cc(OC)c1OC ; CCOc1ccc(-n2cnc(Nc3nc(N4CCC[C@H]4CO)nn4cccc34)c2)cc1OCC ; COc1cc(-n2cnc(Nc3nc(N4CCC[C@H]4CO)nc4cnn(C)c34)c2)cc(OC)c1OC] 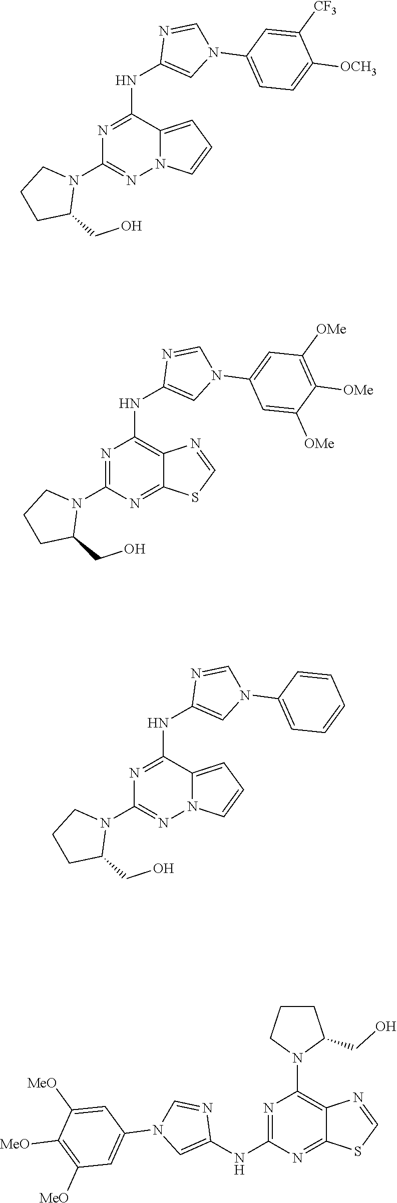 [SMILES: COc1ccc(-n2cnc(Nc3nc(N4CCC[C@H]4CO)nn4cccc34)c2)cc1C(F)(F)F ; COc1cc(-n2cnc(Nc3nc(N4CCC[C@@H]4CO)nc4scnc34)c2)cc(OC)c1OC ; OC[C@@H]1CCCN1c1nc(Nc2cn(-c3ccccc3)cn2)c2cccn2n1 ; COc1cc(-n2cnc(Nc3nc(N4CCC[C@@H]4CO)c4ncsc4n3)c2)cc(OC)c1OC] 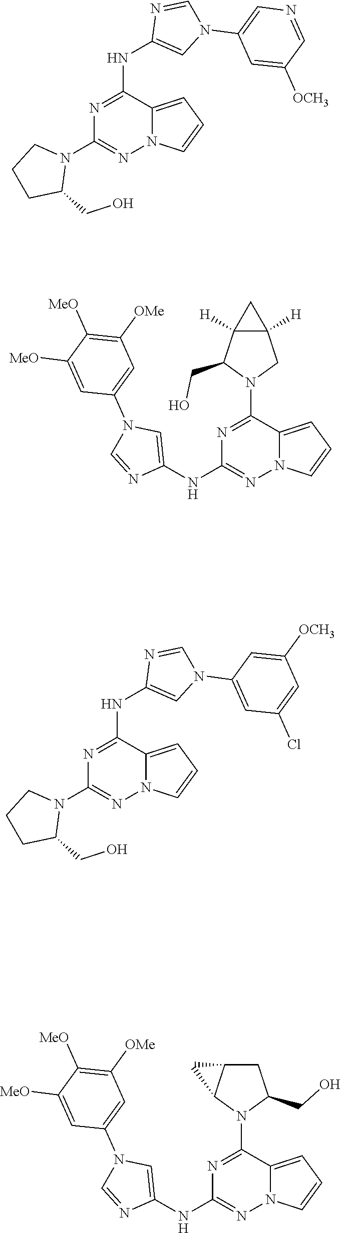 [SMILES: COc1cncc(-n2cnc(Nc3nc(N4CCC[C@H]4CO)nn4cccc34)c2)c1 ; COc1cc(-n2cnc(Nc3nc(N4C[C@@H]5C[C@@H]5[C@@H]4CO)c4cccn4n3)c2)cc(OC)c1OC ; COc1cc(Cl)cc(-n2cnc(Nc3nc(N4CCC[C@H]4CO)nn4cccc34)c2)c1 ; COc1cc(-n2cnc(Nc3nc(N4[C@H](CO)C[C@H]5C[C@H]54)c4cccn4n3)c2)cc(OC)c1OC] 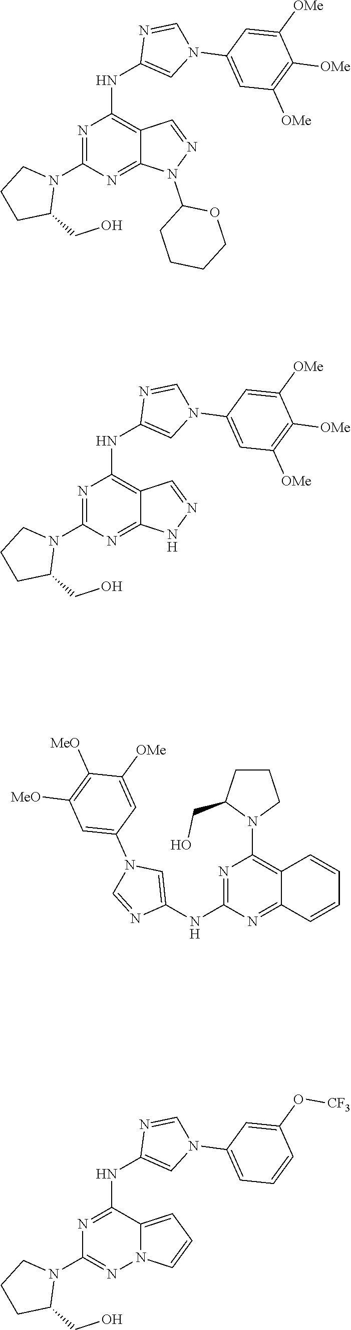 [SMILES: COc1cc(-n2cnc(Nc3nc(N4CCC[C@H]4CO)nc4c3cnn4C3CCCCO3)c2)cc(OC)c1OC ; COc1cc(-n2cnc(Nc3nc(N4CCC[C@H]4CO)nc4[nH]ncc34)c2)cc(OC)c1OC ; COc1cc(-n2cnc(Nc3nc(N4CCC[C@@H]4CO)c4ccccc4n3)c2)cc(OC)c1OC ; OC[C@@H]1CCCN1c1nc(Nc2cn(-c3cccc(OC(F)(F)F)c3)cn2)c2cccn2n1] 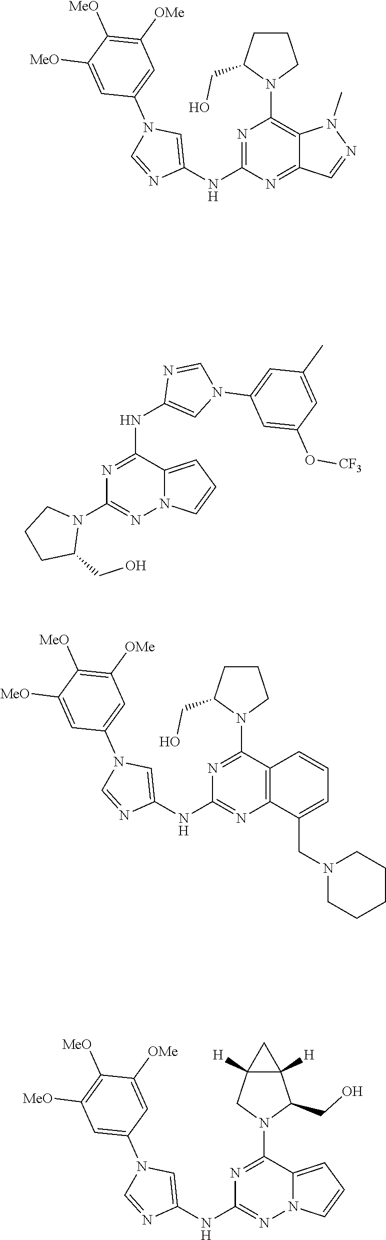 [SMILES: COc1cc(-n2cnc(Nc3nc(N4CCC[C@H]4CO)c4c(cnn4C)n3)c2)cc(OC)c1OC ; Cc1cc(OC(F)(F)F)cc(-n2cnc(Nc3nc(N4CCC[C@H]4CO)nn4cccc34)c2)c1 ; COc1cc(-n2cnc(Nc3nc(N4CCC[C@H]4CO)c4cccc(CN5CCCCC5)c4n3)c2)cc(OC)c1OC ; COc1cc(-n2cnc(Nc3nc(N4C[C@@H]5C[C@@H]5[C@H]4CO)c4cccn4n3)c2)cc(OC)c1OC] 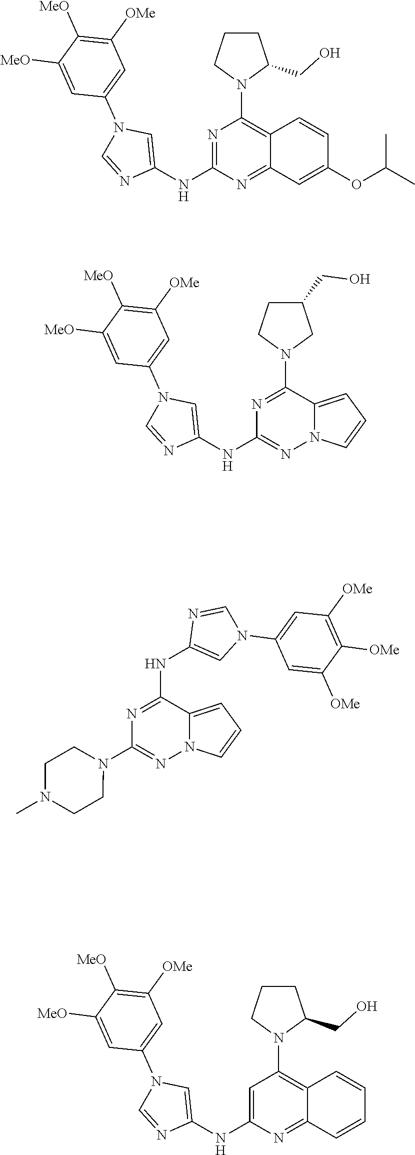 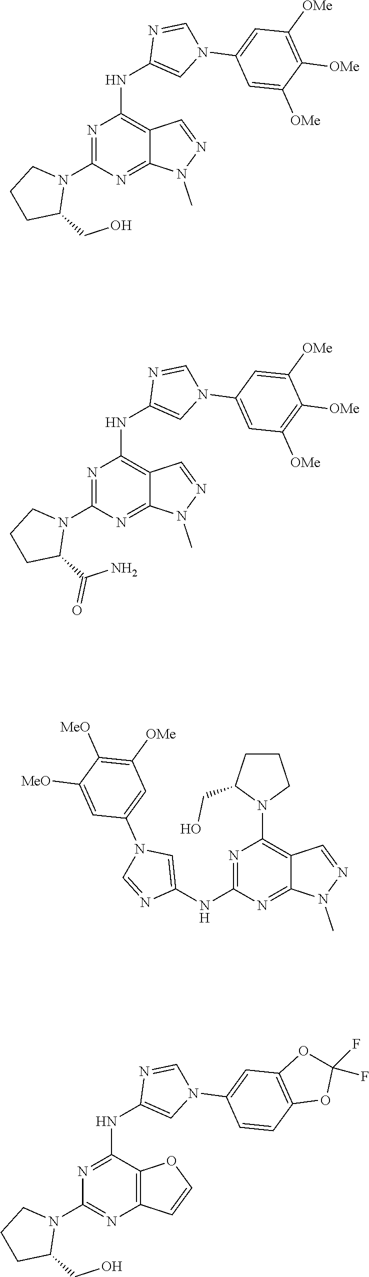 [SMILES: COc1cc(-n2cnc(Nc3nc(N4CCC[C@H]4CO)nc4c3cnn4C)c2)cc(OC)c1OC ; COc1cc(-n2cnc(Nc3nc(N4CCC[C@H]4C(N)=O)nc4c3cnn4C)c2)cc(OC)c1OC ; COc1cc(-n2cnc(Nc3nc(N4CCC[C@H]4CO)c4cnn(C)c4n3)c2)cc(OC)c1OC ; OC[C@@H]1CCCN1c1nc(Nc2cn(-c3ccc4c(c3)OC(F)(F)O4)cn2)c2occc2n1] 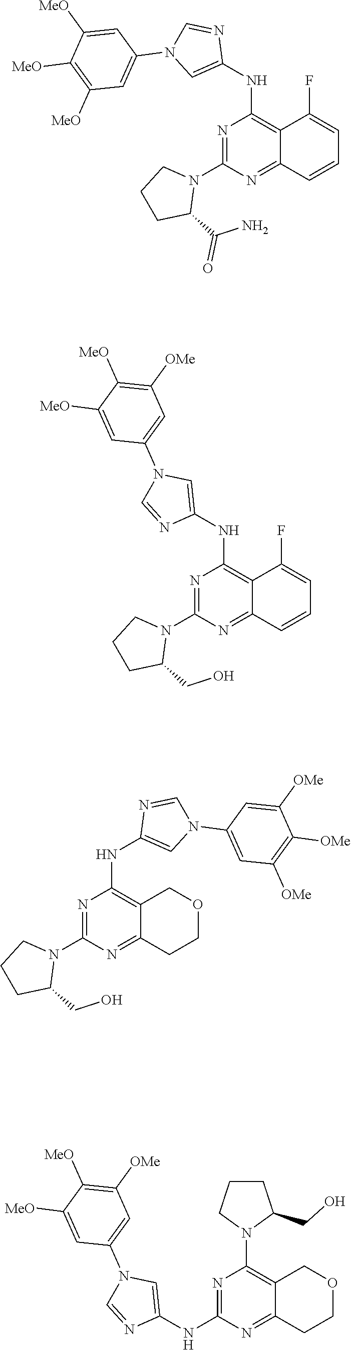 [SMILES: COc1cc(-n2cnc(Nc3nc(N4CCC[C@H]4C(N)=O)nc4cccc(F)c34)c2)cc(OC)c1OC ; COc1cc(-n2cnc(Nc3nc(N4CCC[C@H]4CO)nc4cccc(F)c34)c2)cc(OC)c1OC ; COc1cc(-n2cnc(Nc3nc(N4CCC[C@H]4CO)nc4c3COCC4)c2)cc(OC)c1OC ; COc1cc(-n2cnc(Nc3nc4c(c(N5CCC[C@H]5CO)n3)COCC4)c2)cc(OC)c1OC] 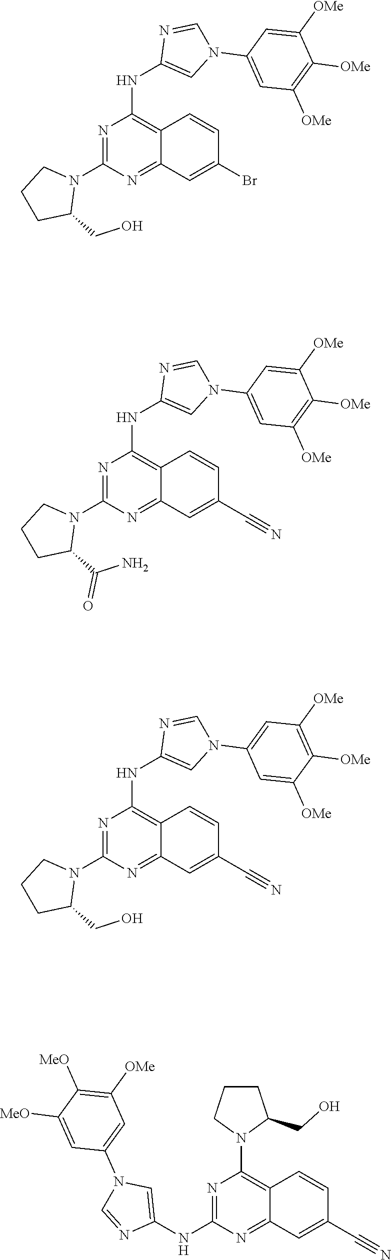 [SMILES: COc1cc(-n2cnc(Nc3nc(N4CCC[C@H]4CO)nc4cc(Br)ccc34)c2)cc(OC)c1OC ; COc1cc(-n2cnc(Nc3nc(N4CCC[C@H]4C(N)=O)nc4cc(C#N)ccc34)c2)cc(OC)c1OC ; COc1cc(-n2cnc(Nc3nc(N4CCC[C@H]4CO)nc4cc(C#N)ccc34)c2)cc(OC)c1OC ; COc1cc(-n2cnc(Nc3nc(N4CCC[C@H]4CO)c4ccc(C#N)cc4n3)c2)cc(OC)c1OC] 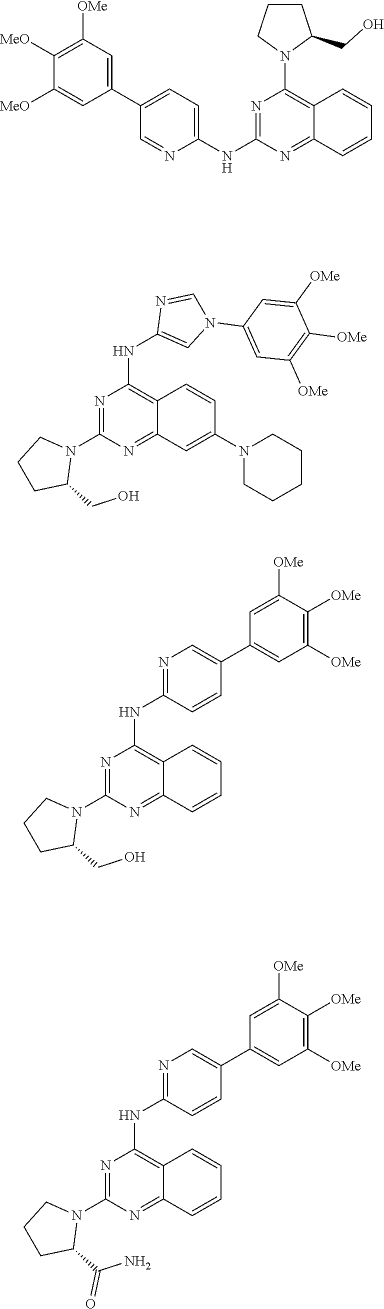 [SMILES: COc1cc(-c2ccc(Nc3nc(N4CCC[C@H]4CO)c4ccccc4n3)nc2)cc(OC)c1OC ; COc1cc(-n2cnc(Nc3nc(N4CCC[C@H]4CO)nc4cc(N5CCCCC5)ccc34)c2)cc(OC)c1OC ; COc1cc(-c2ccc(Nc3nc(N4CCC[C@H]4CO)nc4ccccc34)nc2)cc(OC)c1OC ; COc1cc(-c2ccc(Nc3nc(N4CCC[C@H]4C(N)=O)nc4ccccc34)nc2)cc(OC)c1OC] 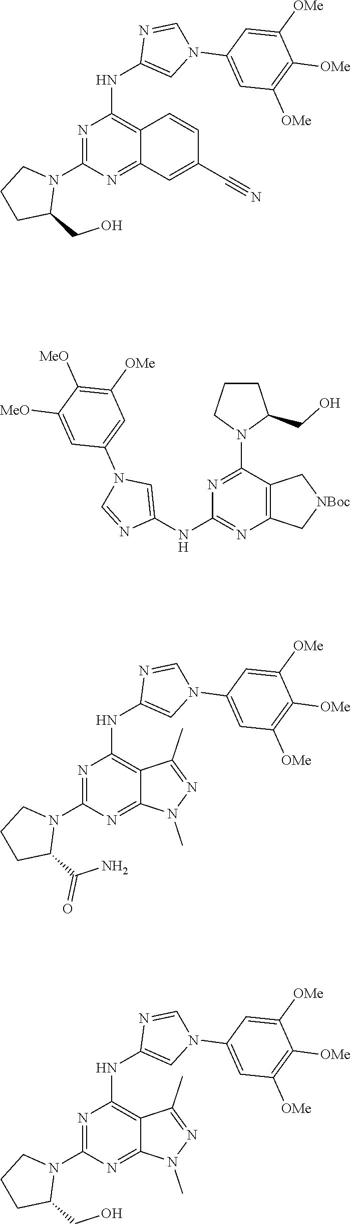 [SMILES: COc1cc(-n2cnc(Nc3nc(N4CCC[C@@H]4CO)nc4cc(C#N)ccc34)c2)cc(OC)c1OC ; COc1cc(-n2cnc(Nc3nc4c(c(N5CCC[C@H]5CO)n3)CN(C(=O)OC(C)(C)C)C4)c2)cc(OC)c1OC ; COc1cc(-n2cnc(Nc3nc(N4CCC[C@H]4C(N)=O)nc4c3c(C)nn4C)c2)cc(OC)c1OC ; COc1cc(-n2cnc(Nc3nc(N4CCC[C@H]4CO)nc4c3c(C)nn4C)c2)cc(OC)c1OC] 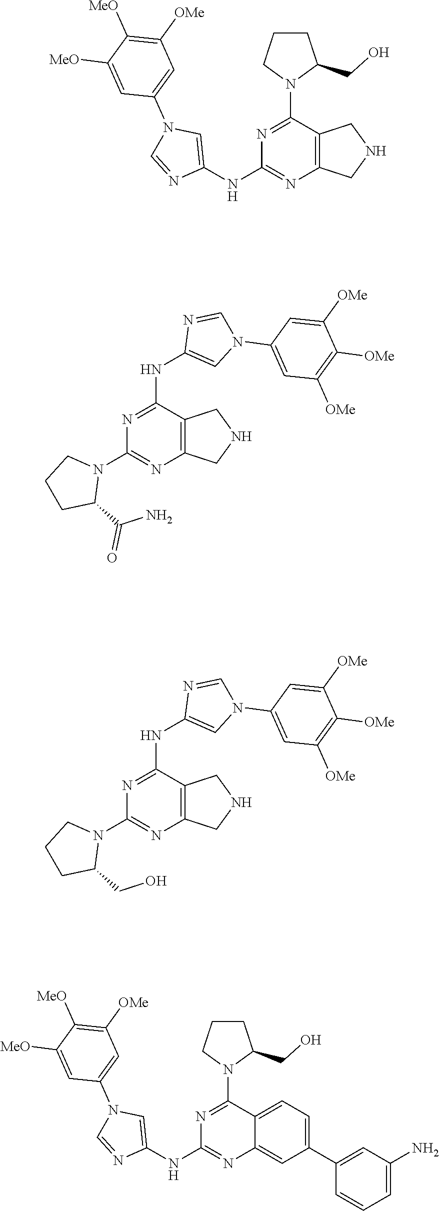 [SMILES: COc1cc(-n2cnc(Nc3nc4c(c(N5CCC[C@H]5CO)n3)CNC4)c2)cc(OC)c1OC ; COc1cc(-n2cnc(Nc3nc(N4CCC[C@H]4C(N)=O)nc4c3CNC4)c2)cc(OC)c1OC ; COc1cc(-n2cnc(Nc3nc(N4CCC[C@H]4CO)nc4c3CNC4)c2)cc(OC)c1OC ; COc1cc(-n2cnc(Nc3nc(N4CCC[C@H]4CO)c4ccc(-c5cccc(N)c5)cc4n3)c2)cc(OC)c1OC] 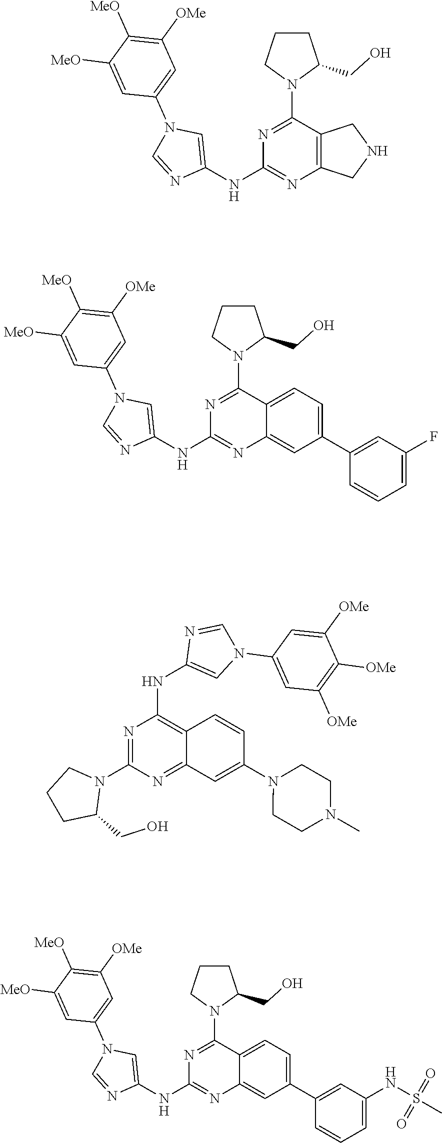 [SMILES: COc1cc(-n2cnc(Nc3nc4c(c(N5CCC[C@@H]5CO)n3)CNC4)c2)cc(OC)c1OC ; COc1cc(-n2cnc(Nc3nc(N4CCC[C@H]4CO)c4ccc(-c5cccc(F)c5)cc4n3)c2)cc(OC)c1OC ; COc1cc(-n2cnc(Nc3nc(N4CCC[C@H]4CO)nc4cc(N5CCN(C)CC5)ccc34)c2)cc(OC)c1OC ; COc1cc(-n2cnc(Nc3nc(N4CCC[C@H]4CO)c4ccc(-c5cccc(NS(C)(=O)=O)c5)cc4n3)c2)cc(OC)c1OC] 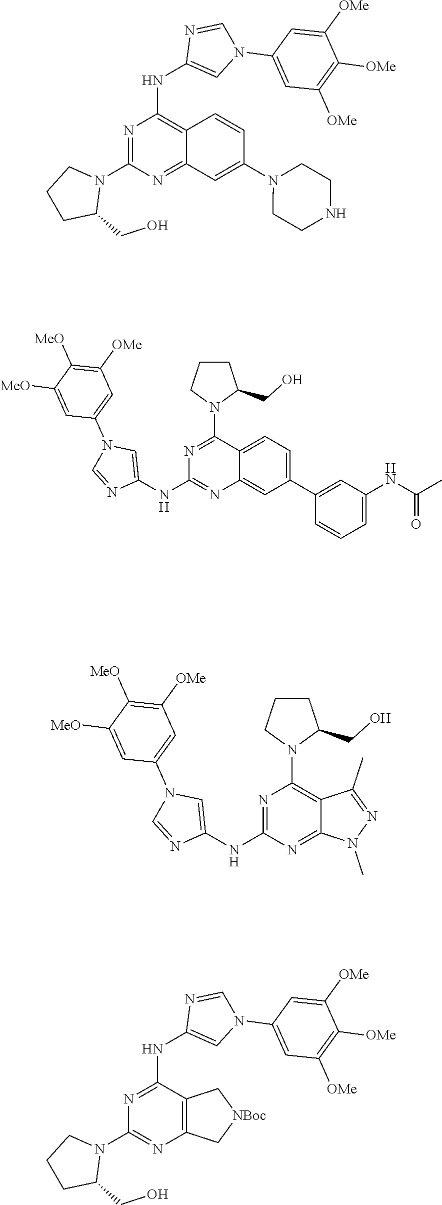 [SMILES: COc1cc(-n2cnc(Nc3nc(N4CCC[C@H]4CO)nc4cc(N5CCNCC5)ccc34)c2)cc(OC)c1OC ; COc1cc(-n2cnc(Nc3nc(N4CCC[C@H]4CO)c4ccc(-c5cccc(NC(C)=O)c5)cc4n3)c2)cc(OC)c1OC ; COc1cc(-n2cnc(Nc3nc(N4CCC[C@H]4CO)c4c(C)nn(C)c4n3)c2)cc(OC)c1OC ; COc1cc(-n2cnc(Nc3nc(N4CCC[C@H]4CO)nc4c3CN(C(=O)OC(C)(C)C)C4)c2)cc(OC)c1OC] 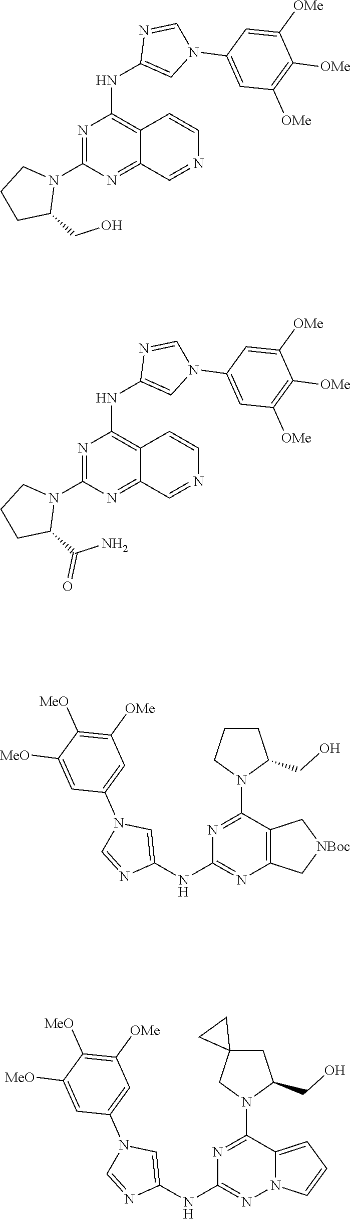 [SMILES: COc1cc(-n2cnc(Nc3nc(N4CCC[C@H]4CO)nc4cnccc34)c2)cc(OC)c1OC ; COc1cc(-n2cnc(Nc3nc(N4CCC[C@H]4C(N)=O)nc4cnccc34)c2)cc(OC)c1OC ; COc1cc(-n2cnc(Nc3nc4c(c(N5CCC[C@@H]5CO)n3)CN(C(=O)OC(C)(C)C)C4)c2)cc(OC)c1OC ; COc1cc(-n2cnc(Nc3nc(N4CC5(CC5)C[C@H]4CO)c4cccn4n3)c2)cc(OC)c1OC] 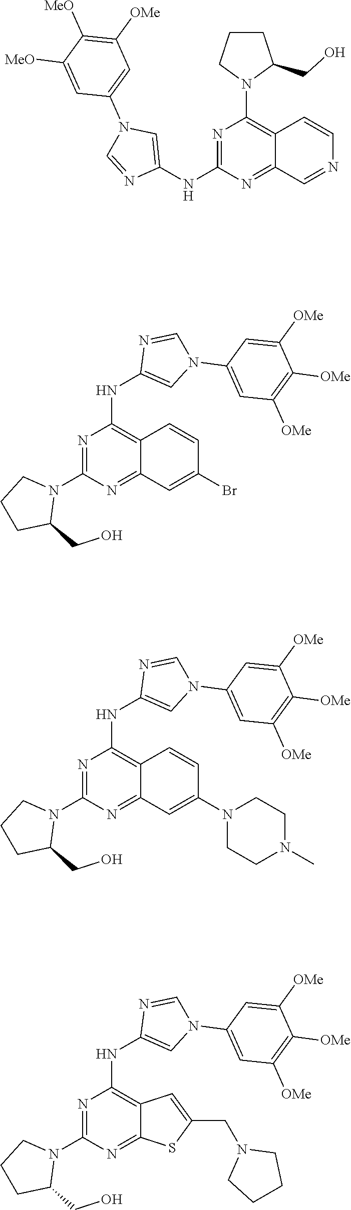 [SMILES: COc1cc(-n2cnc(Nc3nc(N4CCC[C@H]4CO)c4ccncc4n3)c2)cc(OC)c1OC ; COc1cc(-n2cnc(Nc3nc(N4CCC[C@@H]4CO)nc4cc(Br)ccc34)c2)cc(OC)c1OC ; COc1cc(-n2cnc(Nc3nc(N4CCC[C@@H]4CO)nc4cc(N5CCN(C)CC5)ccc34)c2)cc(OC)c1OC ; COc1cc(-n2cnc(Nc3nc(N4CCC[C@H]4CO)nc4sc(CN5CCCC5)cc34)c2)cc(OC)c1OC] 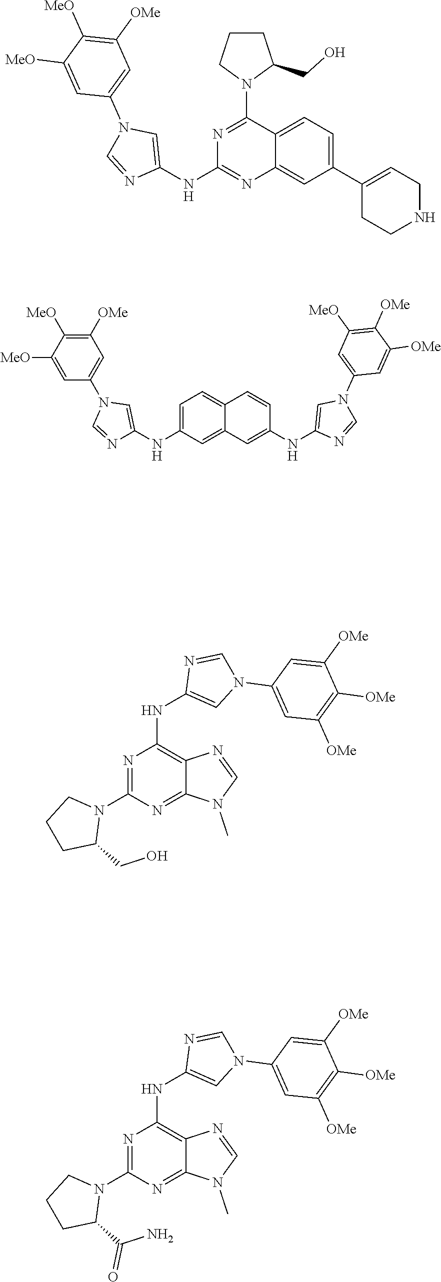 [SMILES: COc1cc(-n2cnc(Nc3nc(N4CCC[C@H]4CO)c4ccc(C5=CCNCC5)cc4n3)c2)cc(OC)c1OC ; COc1cc(-n2cnc(Nc3ccc4ccc(Nc5cn(-c6cc(OC)c(OC)c(OC)c6)cn5)cc4c3)c2)cc(OC)c1OC ; COc1cc(-n2cnc(Nc3nc(N4CCC[C@H]4CO)nc4c3ncn4C)c2)cc(OC)c1OC ; COc1cc(-n2cnc(Nc3nc(N4CCC[C@H]4C(N)=O)nc4c3ncn4C)c2)cc(OC)c1OC] 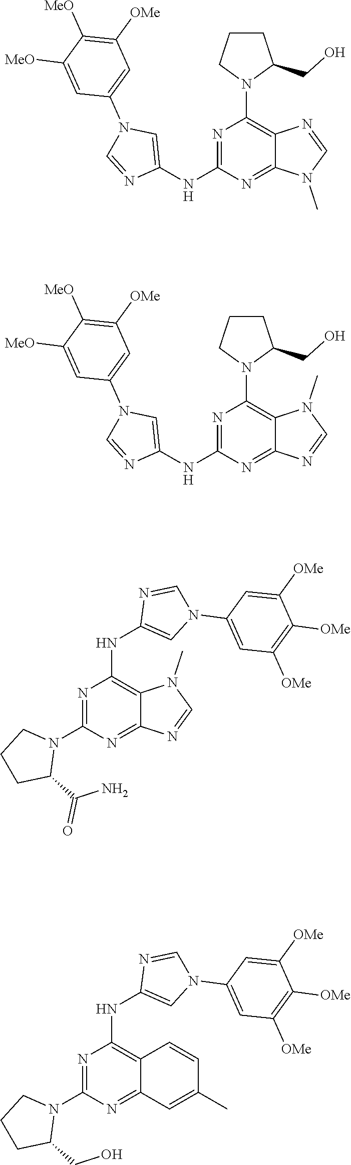 [SMILES: COc1cc(-n2cnc(Nc3nc(N4CCC[C@H]4CO)c4ncn(C)c4n3)c2)cc(OC)c1OC ; COc1cc(-n2cnc(Nc3nc(N4CCC[C@H]4CO)c4c(ncn4C)n3)c2)cc(OC)c1OC ; COc1cc(-n2cnc(Nc3nc(N4CCC[C@H]4C(N)=O)nc4ncn(C)c34)c2)cc(OC)c1OC ; COc1cc(-n2cnc(Nc3nc(N4CCC[C@H]4CO)nc4cc(C)ccc34)c2)cc(OC)c1OC] 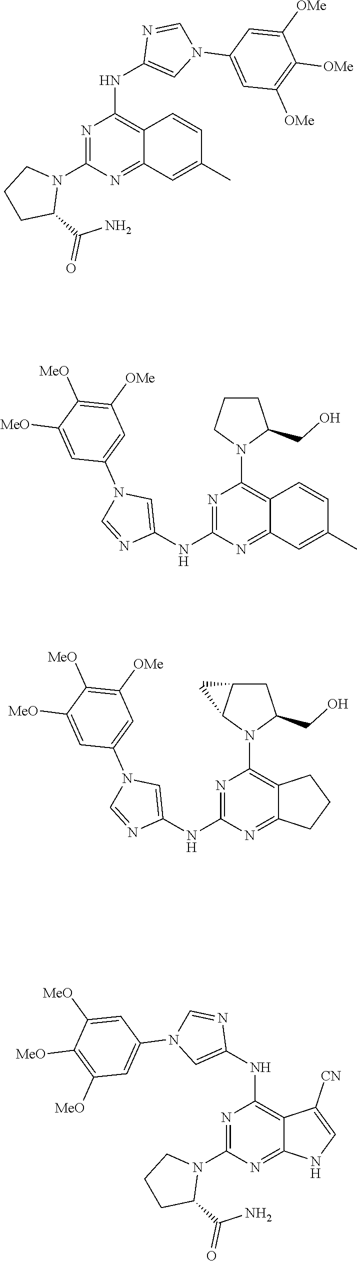 [SMILES: COc1cc(-n2cnc(Nc3nc(N4CCC[C@H]4C(N)=O)nc4cc(C)ccc34)c2)cc(OC)c1OC ; COc1cc(-n2cnc(Nc3nc(N4CCC[C@H]4CO)c4ccc(C)cc4n3)c2)cc(OC)c1OC ; COc1cc(-n2cnc(Nc3nc4c(c(N5[C@H](CO)C[C@H]6C[C@H]65)n3)CCC4)c2)cc(OC)c1OC ; COc1cc(-n2cnc(Nc3nc(N4CCC[C@H]4C(N)=O)nc4[nH]cc(C#N)c34)c2)cc(OC)c1OC] 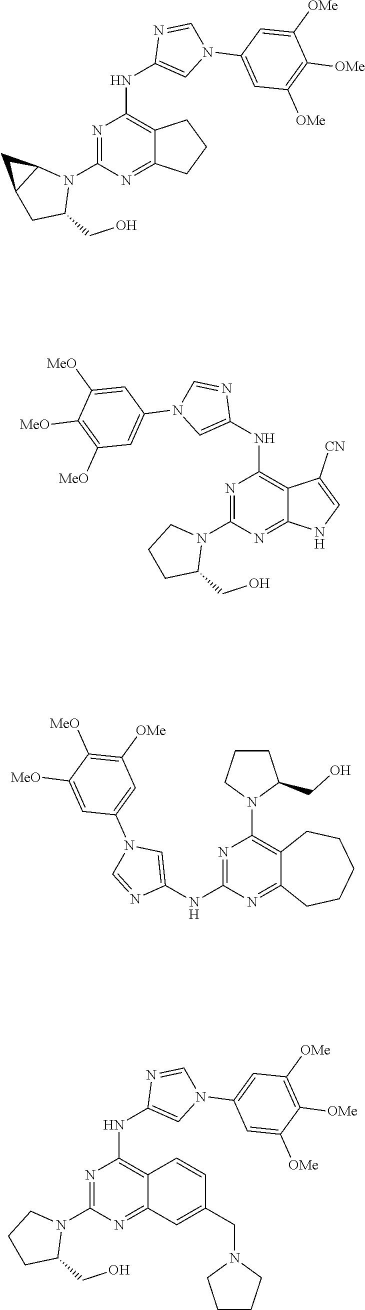 [SMILES: COc1cc(-n2cnc(Nc3nc(N4[C@H](CO)C[C@H]5C[C@H]54)nc4c3CCC4)c2)cc(OC)c1OC ; COc1cc(-n2cnc(Nc3nc(N4CCC[C@H]4CO)nc4[nH]cc(C#N)c34)c2)cc(OC)c1OC ; COc1cc(-n2cnc(Nc3nc4c(c(N5CCC[C@H]5CO)n3)CCCCC4)c2)cc(OC)c1OC ; COc1cc(-n2cnc(Nc3nc(N4CCC[C@H]4CO)nc4cc(CN5CCCC5)ccc34)c2)cc(OC)c1OC] 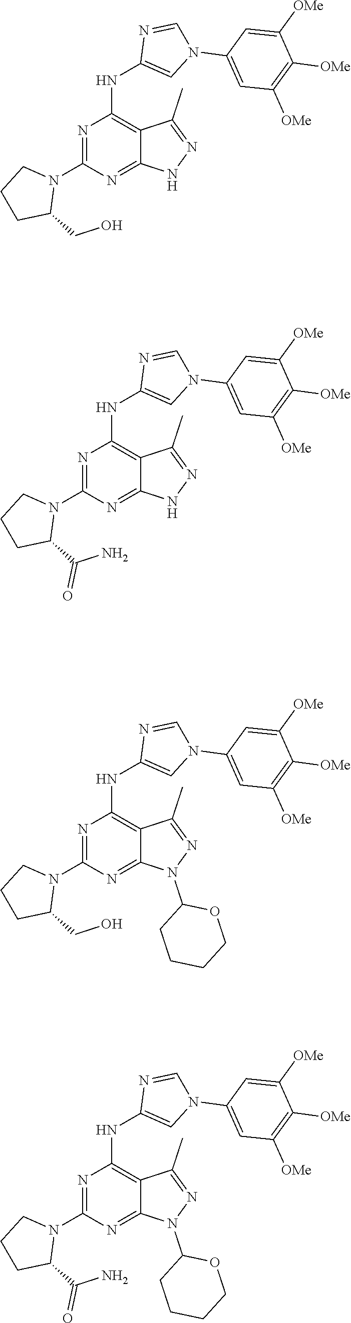 [SMILES: COc1cc(-n2cnc(Nc3nc(N4CCC[C@H]4CO)nc4[nH]nc(C)c34)c2)cc(OC)c1OC ; COc1cc(-n2cnc(Nc3nc(N4CCC[C@H]4C(N)=O)nc4[nH]nc(C)c34)c2)cc(OC)c1OC ; COc1cc(-n2cnc(Nc3nc(N4CCC[C@H]4CO)nc4c3c(C)nn4C3CCCCO3)c2)cc(OC)c1OC ; COc1cc(-n2cnc(Nc3nc(N4CCC[C@H]4C(N)=O)nc4c3c(C)nn4C3CCCCO3)c2)cc(OC)c1OC] 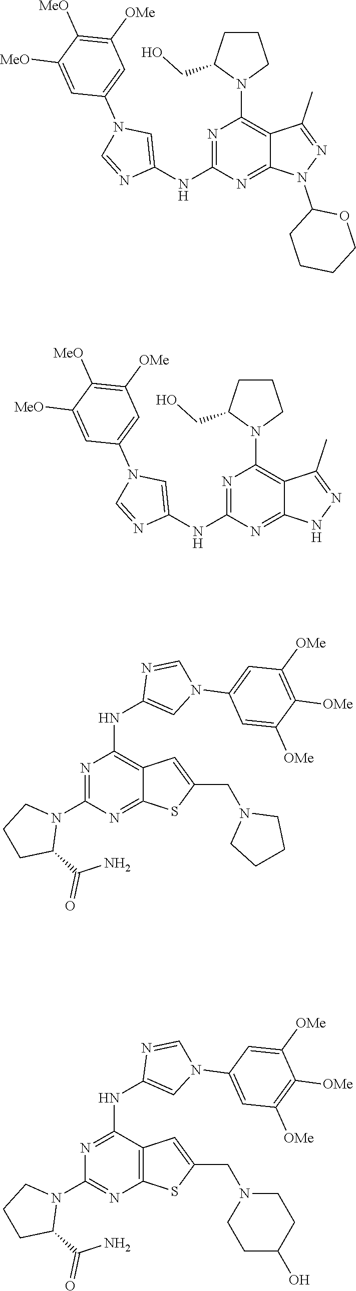 [SMILES: COc1cc(-n2cnc(Nc3nc(N4CCC[C@H]4CO)c4c(C)nn(C5CCCCO5)c4n3)c2)cc(OC)c1OC ; COc1cc(-n2cnc(Nc3nc(N4CCC[C@H]4CO)c4c(C)n[nH]c4n3)c2)cc(OC)c1OC ; COc1cc(-n2cnc(Nc3nc(N4CCC[C@H]4C(N)=O)nc4sc(CN5CCCC5)cc34)c2)cc(OC)c1OC ; COc1cc(-n2cnc(Nc3nc(N4CCC[C@H]4C(N)=O)nc4sc(CN5CCC(O)CC5)cc34)c2)cc(OC)c1OC] 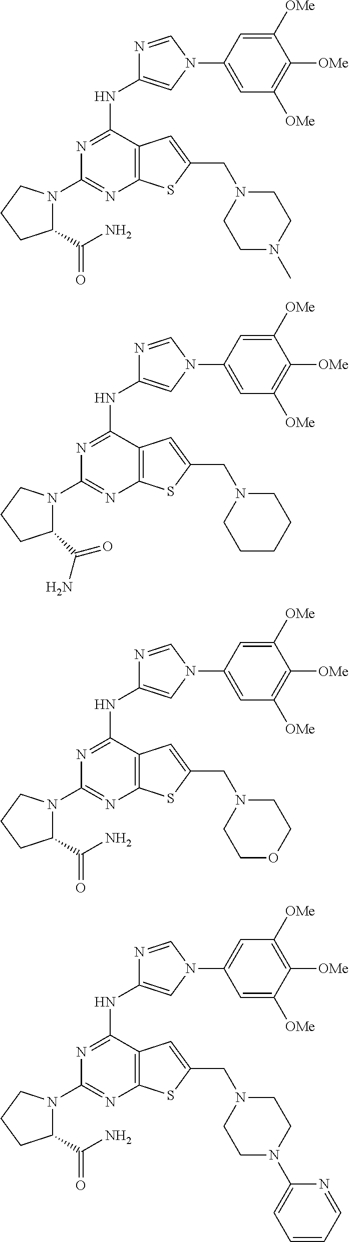 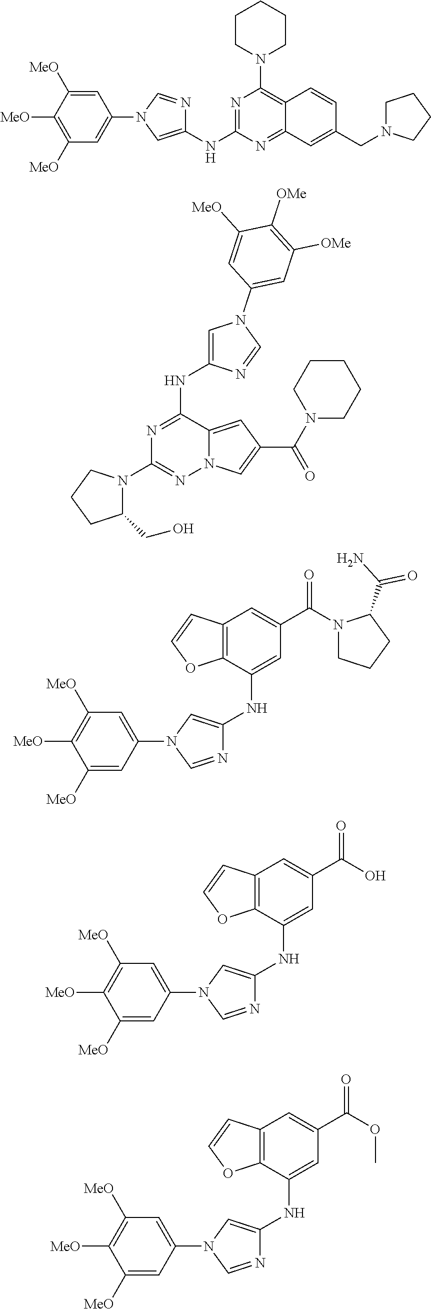 [SMILES: COc1cc(-n2cnc(Nc3nc(N4CCCCC4)c4ccc(CN5CCCC5)cc4n3)c2)cc(OC)c1OC ; COc1cc(-n2cnc(Nc3nc(N4CCC[C@H]4CO)nn4cc(C(=O)N5CCCCC5)cc34)c2)cc(OC)c1OC ; COc1cc(-n2cnc(Nc3cc(C(=O)N4CCC[C@H]4C(N)=O)cc4ccoc34)c2)cc(OC)c1OC ; COc1cc(-n2cnc(Nc3cc(C(=O)O)cc4ccoc34)c2)cc(OC)c1OC ; COC(=O)c1cc(Nc2cn(-c3cc(OC)c(OC)c(OC)c3)cn2)c2occc2c1] 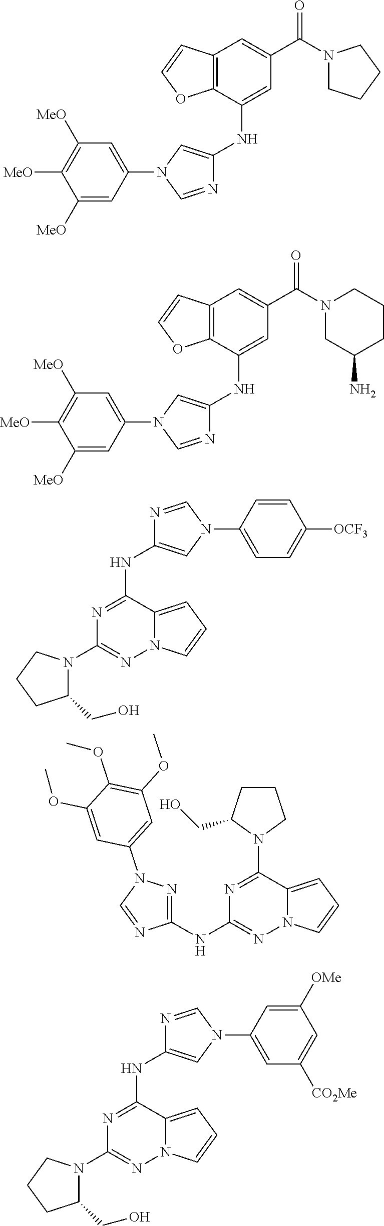 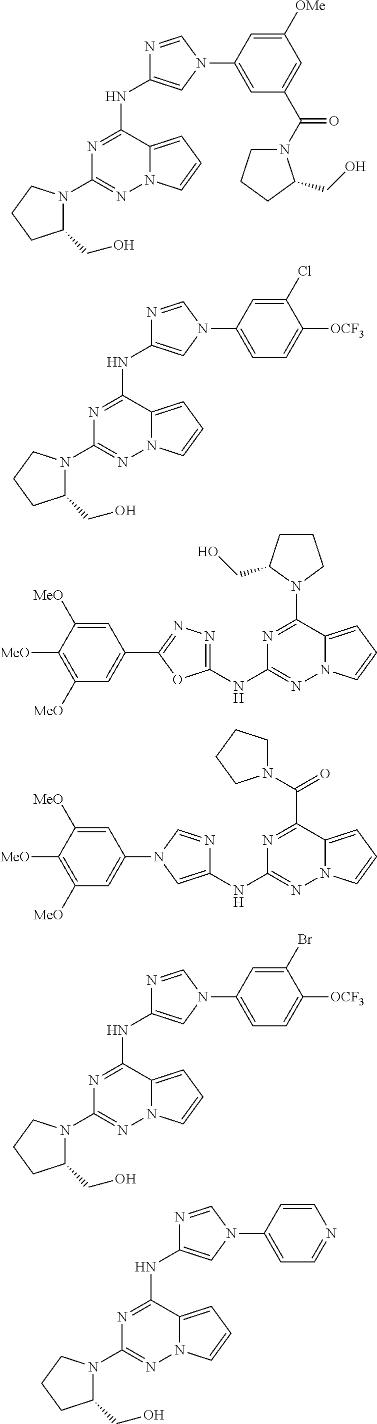 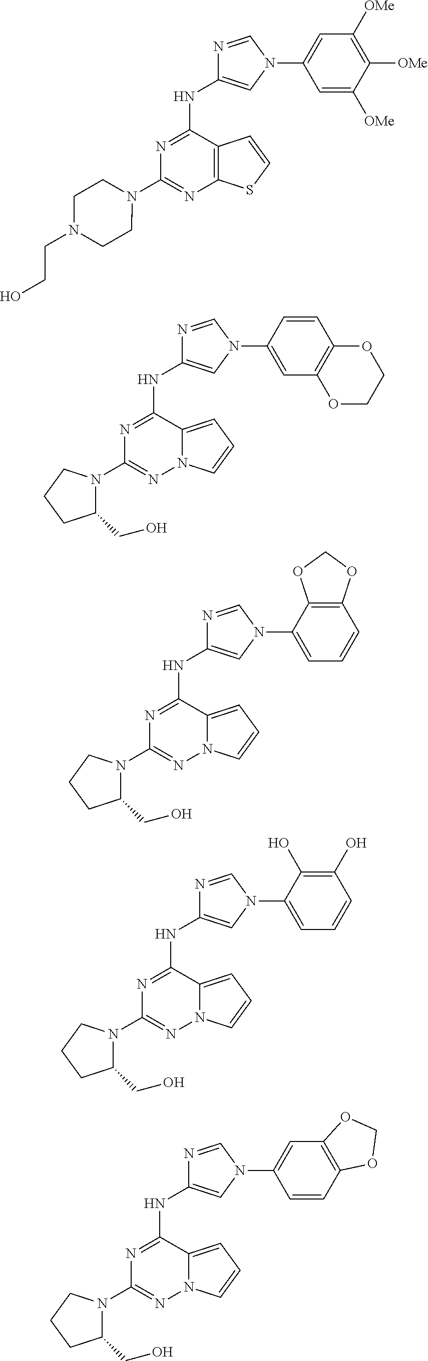 [SMILES: COc1cc(-n2cnc(Nc3nc(N4CCN(CCO)CC4)nc4sccc34)c2)cc(OC)c1OC ; OC[C@@H]1CCCN1c1nc(Nc2cn(-c3ccc4c(c3)OCCO4)cn2)c2cccn2n1 ; OC[C@@H]1CCCN1c1nc(Nc2cn(-c3cccc4c3OCO4)cn2)c2cccn2n1 ; OC[C@@H]1CCCN1c1nc(Nc2cn(-c3cccc(O)c3O)cn2)c2cccn2n1 ; OC[C@@H]1CCCN1c1nc(Nc2cn(-c3ccc4c(c3)OCO4)cn2)c2cccn2n1] 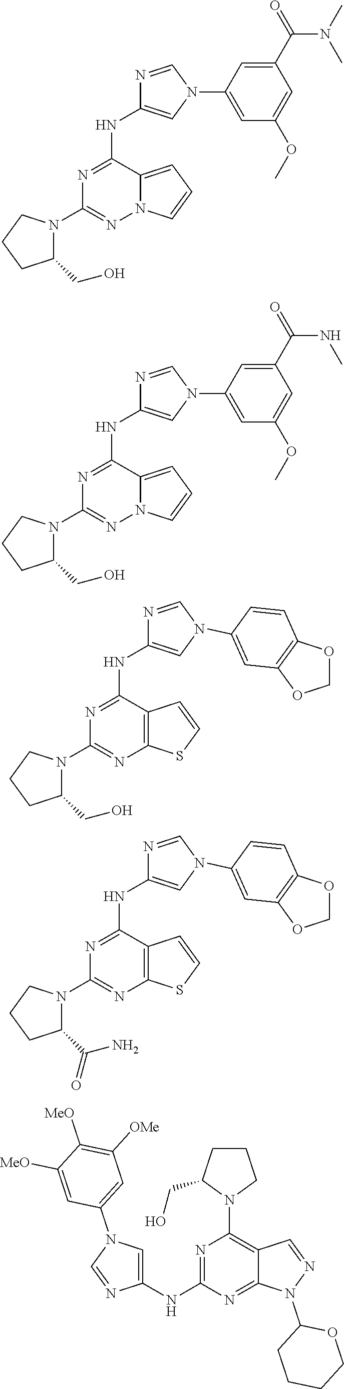 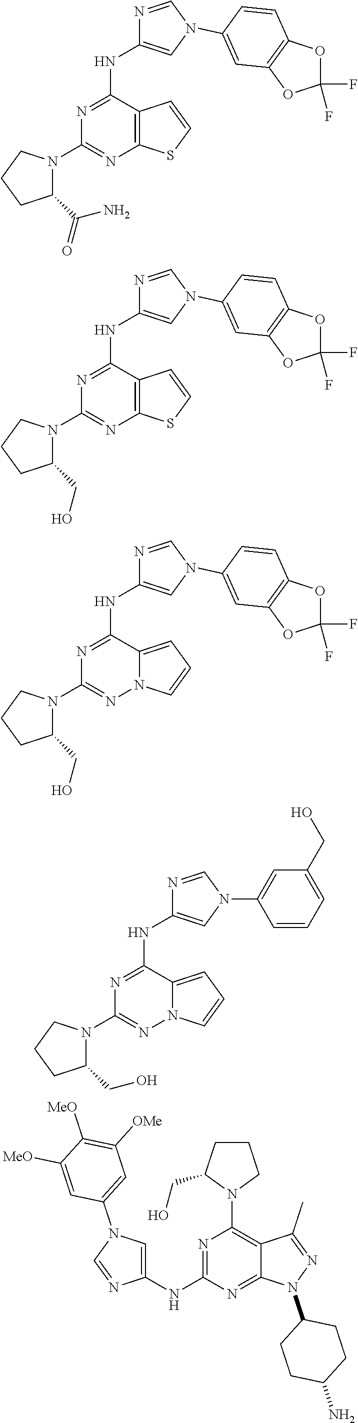 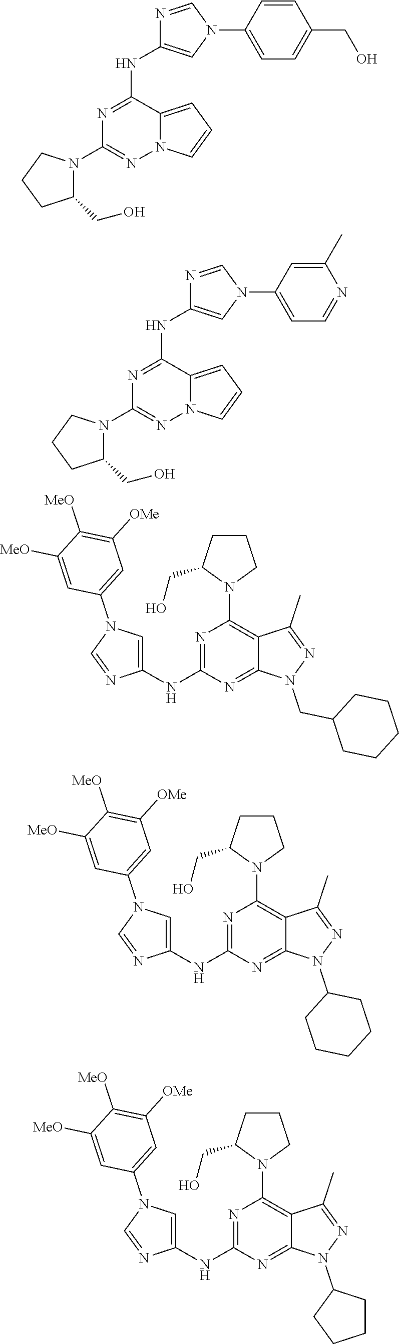 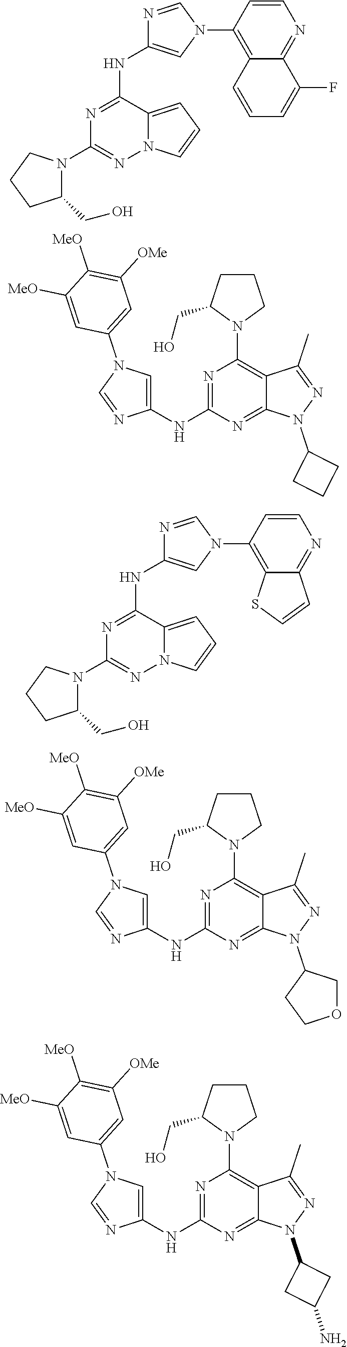 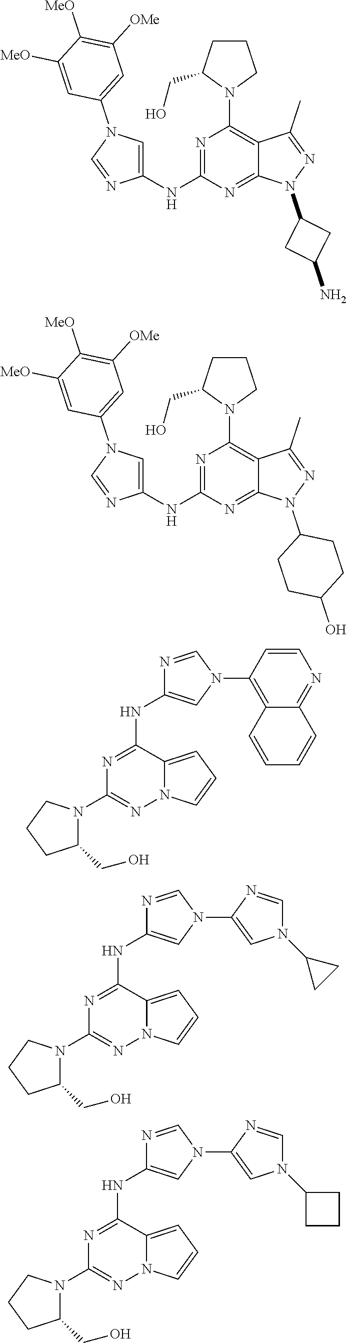 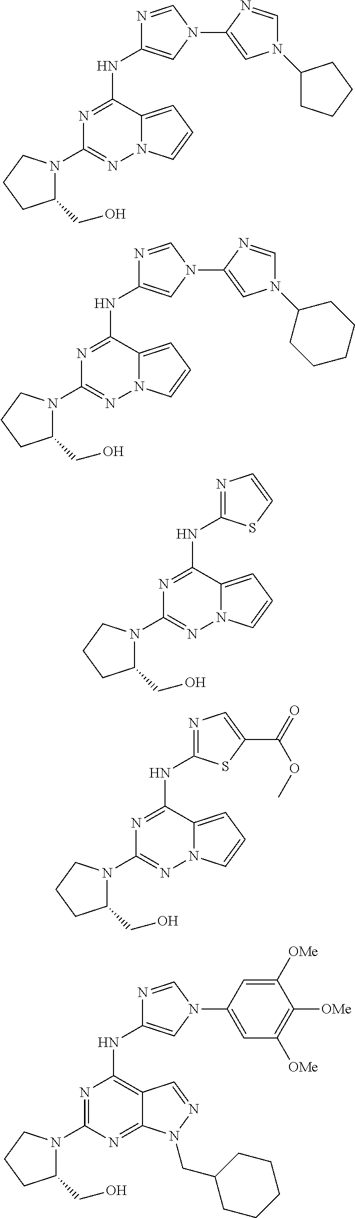 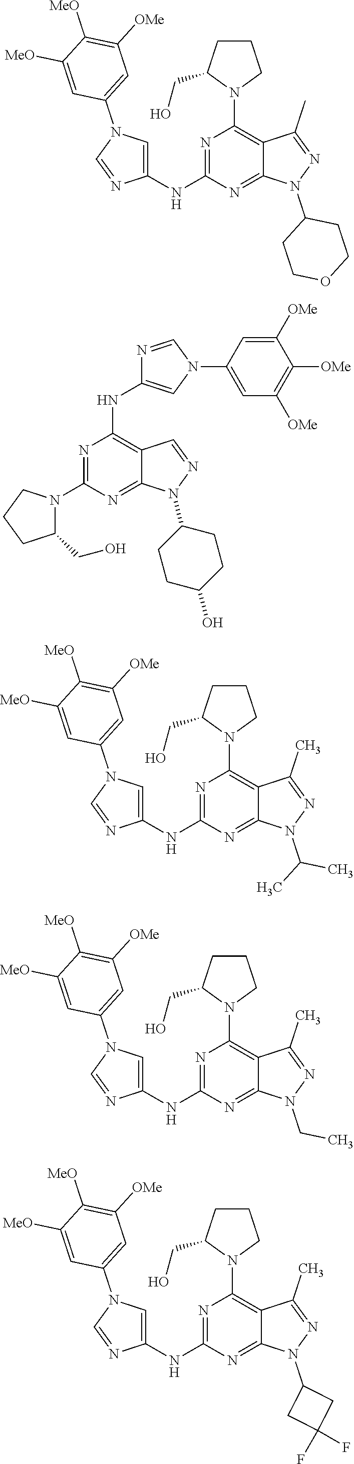 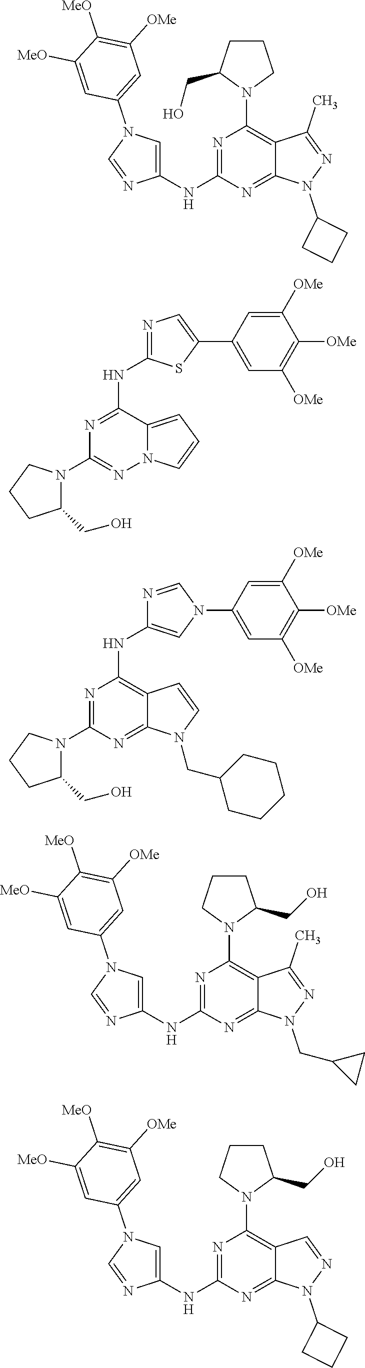 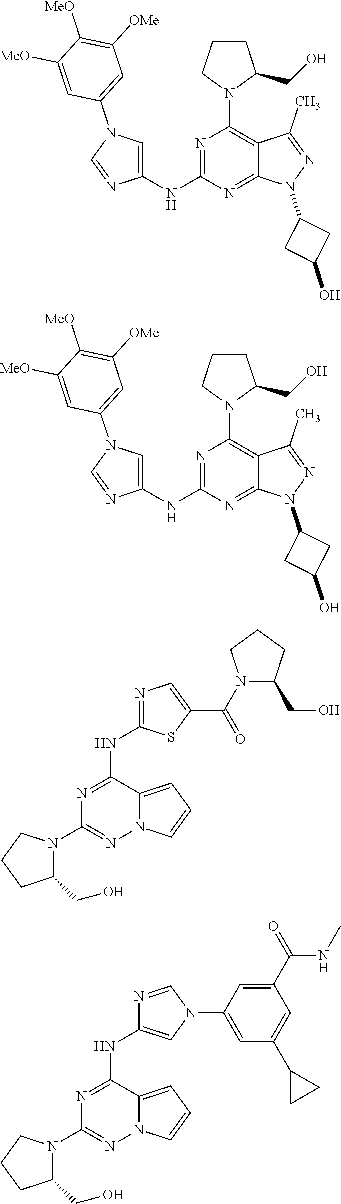 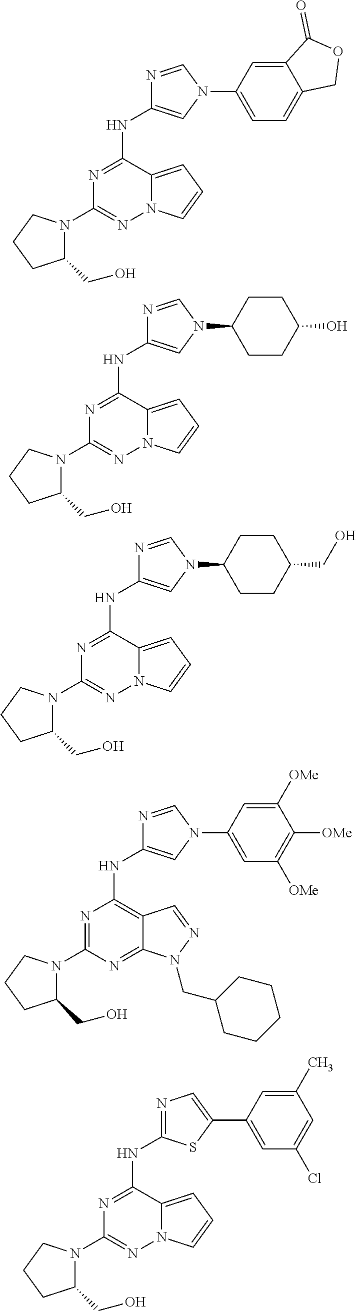 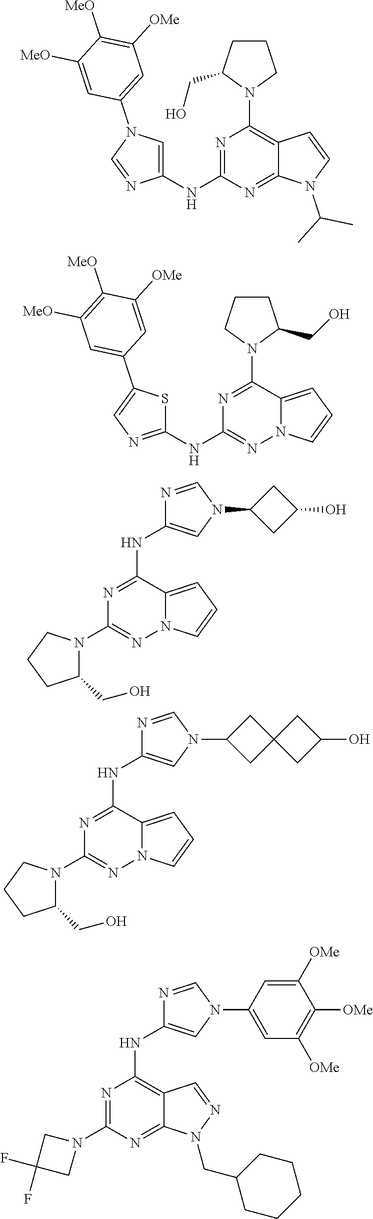 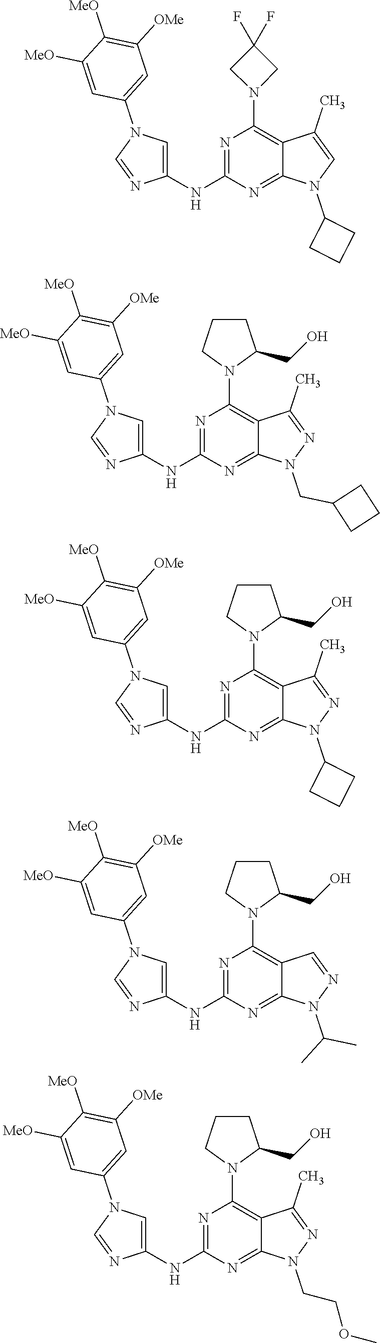 [SMILES: COc1cc(-n2cnc(Nc3nc(N4CC(F)(F)C4)c4c(C)cn(C5CCC5)c4n3)c2)cc(OC)c1OC ; COc1cc(-n2cnc(Nc3nc(N4CCC[C@H]4CO)c4c(C)nn(CC5CCC5)c4n3)c2)cc(OC)c1OC ; COc1cc(-n2cnc(Nc3nc(N4CCC[C@H]4CO)c4c(C)nn(C5CCC5)c4n3)c2)cc(OC)c1OC ; COc1cc(-n2cnc(Nc3nc(N4CCC[C@H]4CO)c4cnn(C(C)C)c4n3)c2)cc(OC)c1OC ; COCCn1nc(C)c2c(N3CCC[C@H]3CO)nc(Nc3cn(-c4cc(OC)c(OC)c(OC)c4)cn3)nc21] 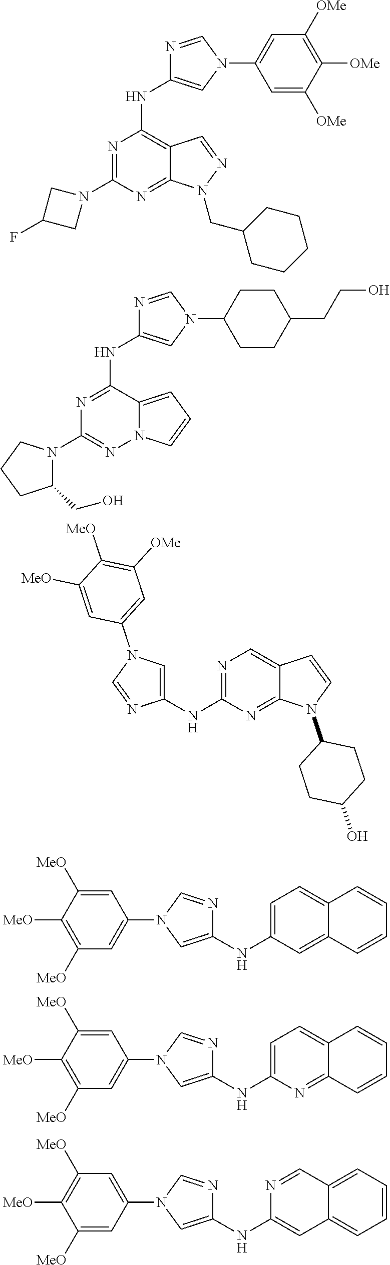 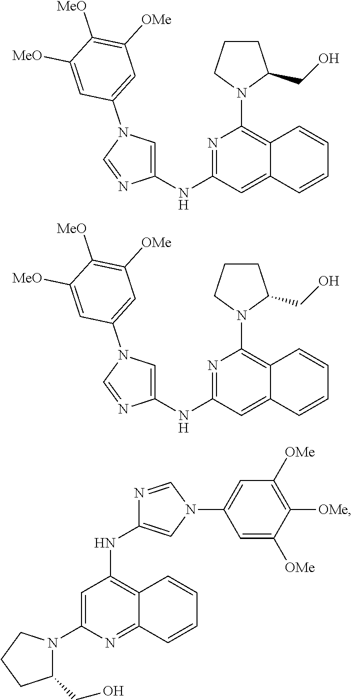
or a pharmaceutically acceptable salt or prodrug thereof.

12. The method of claim 11, wherein the cancer is selected from tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcoma, soft tissue sarcomas, osteosarcomas, peripheral primitive neuroectodermal tumors, retinoblastomas, rhabdomyosarcomas, and Wilms tumors.

13. The method of claim 11, wherein the cancer is a glioma.

14. The method of claim 13, wherein the glioma is diffuse intrinsic pontine glioma.

15. The method of claim 6, wherein the therapeutically effective amount is about 10 to about 500 mg of the compound or a pharmaceutically acceptable salt or prodrug thereof.

16. The method of claim 6, wherein the therapeutically effective amount is about 10 to about 250 mg of the compound or a pharmaceutically acceptable salt or prodrug thereof.

17. The method of claim 6, wherein the compound or a pharmaceutically acceptable salt or prodrug thereof is administered once daily.

18. The method of claim 6, wherein the length of administration is 1 day to 6 months.

19. The method of claim 18, wherein the length of administration is 7 days, 14 days, 21 days, or 28 days.

20-22. (canceled)

23. The method of claim 6, wherein the compound or a pharmaceutically acceptable salt or prodrug thereof is administered as a single oral dosage form.

24. The method of claim 6, wherein the compound or a pharmaceutically acceptable salt or prodrug thereof is administered as a single hard or soft gelatin capsule.

25. (canceled)
